SEQUENCE LISTING (378 sequences)

SEQ ID NO: 1 - protein, 22 aa, Homo sapiens
MLLLVTSLLLCELPHPAFLLIP

SEQ ID NO: 2 - protein, 120 aa, synthetic construct
  REGION 1..120: note = FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 18 aa, synthetic construct
  REGION 1..18: note = Whitlow Linker
GSTSGSGKPGSGEGSTKG

SEQ ID NO: 4 - protein, 107 aa, synthetic construct
  REGION 1..107: note = FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 245 aa, synthetic construct
  REGION 1..245: note = FMC63 scFV
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 94 aa, Homo sapiens
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 10 - protein, 62 aa, Homo sapiens
KKVAKKPTNKAPHPKQEPQEINFPDDLPGSNTAAPVQETLHGCQPVTQEDGKESRISVQERQ

SEQ ID NO: 11 - protein, 42 aa, Homo sapiens
ALYLLRRDQRLPPDAHKPPGGGSFRTPIQEEQADAHSTLAKI

SEQ ID NO: 12 - protein, 25 aa, Homo sapiens
TYCFAPRCRERRRNERLRRESVRPV

SEQ ID NO: 13 - protein, 61 aa, Homo sapiens
KWKKKKRPRNSYKCGTNTMEREESEQTKKREKIHIPERSDEAQRVFKSSKTSSCDKSDTCF

SEQ ID NO: 14 - protein, 48 aa, Homo sapiens
QRRKYRSNKGESPVEPAEPCHYSCPREEEGSTIPIQEDYRKPEPACSP

SEQ ID NO: 15 - protein, 38 aa, Homo sapiens
CWLTKKKYSSSVHDPNGEYMFMRAVNTAKKSRLTDVTL

SEQ ID NO: 16 - protein, 51 aa, Homo sapiens
MGWIRGRRSRHSWEMSEFHNYNLDLKKSDFSTRWQKQRCPVVKSKCRENAS

SEQ ID NO: 17 - protein, 24 aa, Homo sapiens
LCARPRRSPAQEDGKVYINMPGRG

SEQ ID NO: 18 - protein, 52 aa, Homo sapiens
YFLGRLVPRGRGAAEAATRKQRITETESPYQELQGQRSDVYSDLNTQRPYYK

SEQ ID NO: 19 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 20 - protein, 41 aa, Homo sapiens
RSKRSRLLHSDYMNMTPRRPGPTRKHYQPYAPPRDFAAYRS

SEQ ID NO: 21 - protein, 47 aa, Homo sapiens
TTTPAPRPPTPAPTIASQPLSLRPEACRPAAGGAVHTRGLDFACDIY

SEQ ID NO: 22 - protein, 39 aa, Homo sapiens
IEVMYPPPYLDNEKSNGTIIHVKGKHLCPSPLFPGPSKP

SEQ ID NO: 23 - protein, 21 aa, Homo sapiens
IYIWAPLAGTCGVLLLSLVIT

SEQ ID NO: 24 - protein, 27 aa, Homo sapiens
FWVLVVVGGVLACYSLLVTVAFIIFWV

SEQ ID NO: 25 - protein, 15 aa, synthetic construct
  REGION 1..15: note = (GGGGS)3
GGGGSGGGGSGGGGS

SEQ ID NO: 26 - protein, 20 aa, synthetic construct
  REGION 1..20: note = Linker 3
GGSEGKSSGSGSESKSTGGS

SEQ ID NO: 27 - protein, 8 aa, synthetic construct
  REGION 1..8: note = Linker 4
GGGSGGGS

SEQ ID NO: 28 - protein, 12 aa, synthetic construct
  REGION 1..12: note = Linker 5
GGGSGGGSGGGS

SEQ ID NO: 29 - protein, 16 aa, synthetic construct
  REGION 1..16: note = Linker 6
GGGSGGGSGGGSGGGS

SEQ ID NO: 30 - protein, 20 aa, synthetic construct
  REGION 1..20: note = Linker 7
GGGSGGGSGGGSGGGSGGGS

SEQ ID NO: 31 - protein, 20 aa, synthetic construct
  REGION 1..20: note = Linker 8
GGGGSGGGGSGGGGSGGGGS

SEQ ID NO: 32 - protein, 25 aa, synthetic construct
  REGION 1..25: note = Linker 9
GGGGSGGGGSGGGGSGGGGSGGGGS

SEQ ID NO: 33 - protein, 14 aa, synthetic construct
  REGION 1..14: note = Linker 10
IRPRAIGGSKPRVA

SEQ ID NO: 34 - protein, 15 aa, synthetic construct
  REGION 1..15: note = Linker 11
GKGGSGKGGSGKGGS

SEQ ID NO: 35 - protein, 15 aa, synthetic construct
  REGION 1..15: note = Linker 12
GGKGSGGKGSGGKGS

SEQ ID NO: 36 - protein, 15 aa, synthetic construct
  REGION 1..15: note = Linker 13
GGGKSGGGKSGGGKS

SEQ ID NO: 37 - protein, 15 aa, synthetic construct
  REGION 1..15: note = Linker 14
GKGKSGKGKSGKGKS

SEQ ID NO: 38 - protein, 15 aa, synthetic construct
  REGION 1..15: note = Linker 15
GGGKSGGKGSGKGGS

SEQ ID NO: 39 - protein, 15 aa, synthetic construct
  REGION 1..15: note = Linker 16
GKPGSGKPGSGKPGS

SEQ ID NO: 40 - protein, 20 aa, synthetic construct
  REGION 1..20: note = Linker 17
GKPGSGKPGSGKPGSGKPGS

SEQ ID NO: 41 - protein, 20 aa, synthetic construct
  REGION 1..20: note = Linker 18
GKGKSGKGKSGKGKSGKGKS

SEQ ID NO: 42 - protein, 14 aa, synthetic construct
  REGION 1..14: note = Linker 19
STAGDTHLGGEDFD

SEQ ID NO: 43 - protein, 15 aa, synthetic construct
  REGION 1..15: note = Linker 20
GEGGSGEGGSGEGGS

SEQ ID NO: 44 - protein, 15 aa, synthetic construct
  REGION 1..15: note = Linker 21
GGEGSGGEGSGGEGS

SEQ ID NO: 45 - protein, 15 aa, synthetic construct
  REGION 1..15: note = Linker 22
GEGESGEGESGEGES

SEQ ID NO: 46 - protein, 15 aa, synthetic construct
  REGION 1..15: note = Linker 23
GGGESGGEGSGEGGS

SEQ ID NO: 47 - protein, 20 aa, synthetic construct
  REGION 1..20: note = Linker 24
GEGESGEGESGEGESGEGES

SEQ ID NO: 48 - protein, 18 aa, synthetic construct
  REGION 1..18: note = Linker 25
GSTSGSGKPGSGEGSTKG

SEQ ID NO: 49 - protein, 19 aa, synthetic construct
  REGION 1..19: note = Linker 26
PRGASKSGSASQTGSAPGS

SEQ ID NO: 50 - protein, 19 aa, synthetic construct
  REGION 1..19: note = Linker 27
GTAAAGAGAAGGAAAGAAG

SEQ ID NO: 51 - protein, 19 aa, synthetic construct
  REGION 1..19: note = Linker 28
GTSGSSGSGSGGSGSGGGG

SEQ ID NO: 52 - protein, 20 aa, synthetic construct
  REGION 1..20: note = Linker 29
GKPGSGKPGSGKPGSGKPGS

SEQ ID NO: 53 - protein, 4 aa, synthetic construct
  REGION 1..4: note = Linker 30
GSGS

SEQ ID NO: 54 - protein, 10 aa, synthetic construct
  REGION 1..10: note = Linker 31
APAPAPAPAP

SEQ ID NO: 55 - protein, 20 aa, synthetic construct
  REGION 1..20: note = Linker 32
APAPAPAPAPAPAPAPAPAP

SEQ ID NO: 56 - protein, 32 aa, synthetic construct
  REGION 1..32: note = Linker 33
AEAAAKEAAAKEAAAAKEAAAAKEAAAAKAAA

SEQ ID NO: 57 - protein, 15 aa, Homo sapiens
EPKSCDKTHTCPPCP

SEQ ID NO: 58 - protein, 12 aa, Homo sapiens
ERKCCVECPPCP

SEQ ID NO: 59 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 60 - protein, 12 aa, Homo sapiens
ESKYGPPCPSCP

SEQ ID NO: 61 - protein, 486 aa, synthetic construct
  REGION 1..486: note = anti-CD19 scFv CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1461 bp, synthetic construct
  misc_feature 1..1461: note = anti-CD19 scFv CAR
  source 1..1461: mol_type = other DNA
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagatga

SEQ ID NO: 63 - DNA, 1724 bp, synthetic construct
  misc_feature 1..1724: note = CAG Promoter
  source 1..1724: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaa

SEQ ID NO: 64 - DNA, 121 bp, synthetic construct
  misc_feature 1..121: note = SV40 terminator/polyadenylation
  source 1..121: mol_type = other DNA
aacttgtttattgcagcttataatggttacaaataaagcaatagcatcacaaatttcacaaataaagcatttttttcactgcattctagttgtggtttgtccaaactcatcaatgtatctt

SEQ ID NO: 65 - protein, 335 aa, Homo sapiens
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 66 - protein, 504 aa, synthetic construct
  REGION 1..504: note = HLA-G signal-Peptide-B2M-HLA-E
MVVMAPRTLFLLLSGALTLTETWAVMAPRTLILGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 67 - DNA, 1515 bp, synthetic construct
  misc_feature 1..1515: note = HLA-G signal-Peptide-B2M-HLA-E
  source 1..1515: mol_type = other DNA
atggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 68 - protein, 312 aa, Homo sapiens
HSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 69 - protein, 471 aa, synthetic construct
  REGION 1..471: note = HLA-G signal-Peptide-B2M-HLA-G
MVVMAPRTLFLLLSGALTLTETWARIIPRHLQLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 70 - DNA, 1422 bp, synthetic construct
  misc_feature 1..1422: note = HLA-G signal-Peptide-B2M-HLA-G
  source 1..1422: mol_type = other DNA
gccaccatggtggtcatggcgccccgaaccctcttcctgctgctctcgggggccctgaccctgaccgagacctgggcgcggatcattccccgacatctgcaactgggaggcggcggttcaggagggggcggatcgatccaacgcacccccaagatccaggtctactccagacacccggccgaaaacggaaagtcgaacttcctgaactgctatgtgtcaggattccacccgtccgacatcgaggtggacctcctgaagaacggcgaacgcattgagaaggtcgagcactccgatctgtcgttctccaaggactggtccttctaccttctctactataccgaattcaccccgaccgagaaggacgaatacgcctgccgggtcaaccacgtgaccctgagccagccaaagatcgtgaaatgggaccgcgatatgggaggaggaggttccggcggaggaggaagcggaggcggaggttccggctcccactccatgaggtatttcagcgccgccgtgtcccggcctggccgcggagagcctcgcttcatcgccatgggatacgtggacgacacccagttcgtcagattcgacagcgacagcgcctgtcctcggatggaacctagagcaccttgggtcgagcaagagggccctgagtactgggaagaagagacacggaacaccaaggctcacgcccagaccgacagaatgaacctgcagaccctgcggggctactacaatcagtctgaggccagcagccatactctgcagtggatgatcggctgcgatctgggctctgatggcagactgctgagaggctacgagcagtacgcctacgacggcaaggattatctggccctgaacgaggacctgcggtcttggacagctgccgatacagccgctcagatcagcaagagaaagtgcgaggccgccaatgtggccgaacagagaagggcttacctggaaggcacctgtgtggaatggctgcacagatacctggaaaacggcaaagagatgctgcagcgggccgatcctcctaagacacatgtgacccaccatcctgtgttcgactacgaggccacactgagatgttgggccctgggcttttaccctgccgagatcatcctgacctggcagcgagatggcgaggatcagacccaggatgtggaactggtggaaaccagacctgccggcgacggcacctttcagaaatgggctgctgtggtggtgcccagcggagaggaacagagatacacctgtcacgtgcagcacgagggactgcctgaacctctgatgctgagatggaagcagagcagcctgcctacaatccccatcatgggaatcgtggccggactggtggttctggccgctgttgttacaggtgctgcagtggctgccgtgctgtggcggaagaaaagcagcgactga

SEQ ID NO: 71 - protein, 371 aa, synthetic construct
  REGION 1..371: note = truncated EGFR
MRPSGTAGAALLALLAALCPASRAGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 72 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 73 - protein, 19 aa, synthetic construct
  REGION 1..19: note = P2A
ATNFSLLKQAGDVEENPGP

SEQ ID NO: 74 - protein, 556 aa, synthetic construct
  REGION 1..556: note = tEGFR-P2A-IL15
MRPSGTAGAALLALLAALCPASRAGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - DNA, 1668 bp, synthetic construct
  misc_feature 1..1668: note = tEGFR-P2A-IL15
  source 1..1668: mol_type = other DNA
atgaggccctcaggcactgccggggccgccctcctggccctgttagccgctttgtgtccagcaagccgcgccggagtgcggaaatgtaagaaatgcgaaggaccctgccggaaggtatgcaacggcattgggattggcgaattcaaggacagcctgagcattaatgctacaaacatcaagcactttaagaattgcaccagcattagcggcgatctgcatatactgccagtggctttccgaggcgactcttttactcatacccctccgctggaccctcaagagctggacattctcaagactgtgaaggaaattacggggtttctgctcattcaggcctggcctgaaaaccgcacggatttgcatgcctttgagaatctggaaataatcagaggccggacgaaacagcatggccagttcagcctcgcggtcgtctctttgaatattacgtcactcggcctcaggtccctcaaagagatttctgatggcgatgtcatcatctctggtaataagaatctgtgttacgcaaataccatcaattggaagaagctctttgggacctcaggtcaaaagactaaaattatctccaaccgcggcgagaacagctgtaaggctacaggccaggtttgccacgcgctctgctccccagagggttgctgggggcctgagccaagggattgcgtttcatgtcgcaacgtgtctcggggcagagaatgcgtggataaatgtaacctcttagagggcgaacctcgcgagtttgttgagaactcagaatgtatacagtgccaccccgaatgtcttcctcaggccatgaatatcacatgcaccggacgcggaccagacaactgtatccaatgtgctcactacattgacggacctcattgtgtgaaaacatgccccgcaggagttatgggagaaaacaacaccctcgtttggaaatatgccgatgcaggtcacgtatgtcacctgtgccacccaaactgcacttatgggtgcaccgggccgggcctggaggggtgccctacgaatggaccaaaaattcccagtattgcaactgggatggtcggggcactgttgttgctgcttgtggttgccctcgggataggcctgtttatgtctggctccggcgccaccaatttcagcctgctgaaacaggcaggcgacgtcgaagaaaatccaggaccaatgcgaatatcaaaaccacacttgcgcagcatttctatacagtgctatttgtgcttgttgctgaactctcacttcctcacagaggctgggatacacgttttcatacttggatgtttttcagctgggctgccgaagacagaggcgaattgggtgaatgtaatttcagacctcaagaagatcgaggatctcatccagtccatgcacatcgacgctactctgtacacagagagcgatgtccacccttcttgtaaggttaccgccatgaaatgcttccttttggaactccaagtcatctcattggaatcaggggatgcgtccattcatgacaccgtggaaaacctgataatactggctaacaacagcttgtcaagtaatgggaatgttactgagtccggttgtaaagaatgtgaagagctggaggagaagaacattaaggaatttttgcaatcttttgtacatattgttcagatgtttattaacacaagc

SEQ ID NO: 76 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 77 - protein, 19 aa, synthetic construct
  REGION 1..19: note = Signal Sequence
MEFGLSWVFLVALFRGVQC

SEQ ID NO: 78 - protein, 21 aa, synthetic construct
  REGION 1..21: note = Polivy epitope
ARSEDRYRNPKGSACSRIWQS

SEQ ID NO: 79 - protein, 287 aa, synthetic construct
  REGION 1..287: note = CD79b-P2A-IL15
MEFGLSWVFLVALFRGVQCARSEDRYRNPKGSACSRIWQSTTTPAPRPPTPAPTIASQPLSLRPEACRPAAGGAVHTRGLDFACDIYIWAPLAGTCGVLLLSLVITATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 80 - protein, 10 aa, synthetic construct
  REGION 1..10: note = CD20 mimitope
ACPYANPSLC

SEQ ID NO: 81 - protein, 296 aa, synthetic construct
  REGION 1..296: note = CD20 mimitope-P2A-IL15
MEFGLSWVFLVALFRGVQCACPYANPSLCGGGGSGGGGSACPYANPSLCTTTPAPRPPTPAPTIASQPLSLRPEACRPAAGGAVHTRGLDFACDIYIWAPLAGTCGVLLLSLVITATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 82 - protein, 176 aa, synthetic construct
  REGION 1..176: note = ErbB epitope
EGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGGGGSGG

SEQ ID NO: 83 - protein, 376 aa, synthetic construct
  REGION 1..376: note = ErbB epitope-P2A-IL15
MEFGLSWVFLVALFRGVQCEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGGGGSGGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 84 - DNA, 1000 bp, synthetic construct
  misc_feature 1..1000: note = CIITA targeting left homology arm
  source 1..1000: mol_type = other DNA
acaggaggtaaccatttaacaagaaagcagagtgatgttagattatagcaagatactgttgactgtagaaggctctgaggctagagagctgctttctataaaacagagtgatcatatattagaagaggtgttaaagacatgttcacaccaagctgagacttcctccttgataccaccaggaggatgggcagagactggaaaagacactaactttctccctatgggagtcagtattatttagcatcactttggcgggtcaccccaaaccatctgactacaagggtaccatatttgggttaacactcttttggtataatttatgttttagtccaatgtcttgggatgaaaatgacaggtgggccacttatgatctccagagaaattcagggcaatttggtgtgggagtaggcatggtagaggagagcagcatctaagaagtccccagcagaggctctcagcttgtcttgaggcatctgggcggagggctatgatactggccccatcctgcagaaggtggcagatattggcagctggcaccagtgcggttccattgtgatcatcatttctgaacgtcagactgttgaaggttcccccaacagactttctgtgcaactttctgtcttcaccaaattcagtccacagtaaggaagtgaaattaatttcagaggtgtggggagggcttaagggagtgtggtaaaattagagggtgttcagaaacagaaatctgaccgcttggggccaccttgcagggagagtttttttgatgatccctcacttgtttctttgcatgttggcttagcttggcgggctcccaactggtgactggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcag

SEQ ID NO: 85 - DNA, 1000 bp, synthetic construct
  misc_feature 1..1000: note = CIITA targeting right homology arm
  source 1..1000: mol_type = other DNA
agccccaaggtaaaaaggccgggaaagcatcttaatttagcgtgcagtctcagctggtcctgccattccagataaacagagaaaccattctgaattggggatgggggtgaggatgggaacaggagtctgtgtcctgctggggcaggccattggaagatgtgaaagagttgtctatttccttccaccggagggagacttcaggtcagccaggtgtctggagtatgaaccatgtatcagcaccgaaaggttctagaagtcagactttcgggcagtgtgtcactaactctcagcatgctggcctggctcggcccacagcaaggtcttctcgcctccctttgggtaaatactgaggggtgcctctgcaggacgggacctctgccagactccactccatacccagagaagcagggaaaccaaaattggagtcagccttgaggtgtagctgttgagccctcagcagctggggagagctggcggatgctgccctccccccagtttcctaatggtgttgtttaaaaagggtcaggggacgggggaacagatggtgggaagagcacagtgcagacacctggcaccggctctgaaggcagcatggcagctacaccgttggctgggaagggtgtgcccctgaagaagtcgtttacattctcgagtcaattttcctggagtgtacaatggacctgtgggaaagcctgtatgaaagggtaatgatgagggacctagcacagtgtccaatattttataggaactggaattgagctcataggagctcaattttattggcattgctgttgttggatggttaaaggggtggtatcccttttctcagactcccctgaaatgtatggtttgctttgaacccagagactgatgacaggtctgccggtgtggttgggtgcagccttaagttgctacgggaaagtgttggagggggagaagtcagaggtaaccttgccccctccctcaattccagatgaggaaattcaggcctgaaaaggga

SEQ ID NO: 86 - DNA, 6604 bp, synthetic construct
  misc_feature 1..6604: note = CIITA targeting plasmid
  source 1..6604: mol_type = other DNA
cgcctgacctctcgaacaggaggtaaccatttaacaagaaagcagagtgatgttagattatagcaagatactgttgactgtagaaggctctgaggctagagagctgctttctataaaacagagtgatcatatattagaagaggtgttaaagacatgttcacaccaagctgagacttcctccttgataccaccaggaggatgggcagagactggaaaagacactaactttctccctatgggagtcagtattatttagcatcactttggcgggtcaccccaaaccatctgactacaagggtaccatatttgggttaacactcttttggtataatttatgttttagtccaatgtcttgggatgaaaatgacaggtgggccacttatgatctccagagaaattcagggcaatttggtgtgggagtaggcatggtagaggagagcagcatctaagaagtccccagcagaggctctcagcttgtcttgaggcatctgggcggagggctatgatactggccccatcctgcagaaggtggcagatattggcagctggcaccagtgcggttccattgtgatcatcatttctgaacgtcagactgttgaaggttcccccaacagactttctgtgcaactttctgtcttcaccaaattcagtccacagtaaggaagtgaaattaatttcagaggtgtggggagggcttaagggagtgtggtaaaattagagggtgttcagaaacagaaatctgaccgcttggggccaccttgcagggagagtttttttgatgatccctcacttgtttctttgcatgttggcttagcttggcgggctcccaactggtgactggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagtcgagaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgccgccaccatgaggccctcaggcactgccggggccgccctcctggccctgttagccgctttgtgtccagcaagccgcgccggagtgcggaaatgtaagaaatgcgaaggaccctgccggaaggtatgcaacggcattgggattggcgaattcaaggacagcctgagcattaatgctacaaacatcaagcactttaagaattgcaccagcattagcggcgatctgcatatactgccagtggctttccgaggcgactcttttactcatacccctccgctggaccctcaagagctggacattctcaagactgtgaaggaaattacggggtttctgctcattcaggcctggcctgaaaaccgcacggatttgcatgcctttgagaatctggaaataatcagaggccggacgaaacagcatggccagttcagcctcgcggtcgtctctttgaatattacgtcactcggcctcaggtccctcaaagagatttctgatggcgatgtcatcatctctggtaataagaatctgtgttacgcaaataccatcaattggaagaagctctttgggacctcaggtcaaaagactaaaattatctccaaccgcggcgagaacagctgtaaggctacaggccaggtttgccacgcgctctgctccccagagggttgctgggggcctgagccaagggattgcgtttcatgtcgcaacgtgtctcggggcagagaatgcgtggataaatgtaacctcttagagggcgaacctcgcgagtttgttgagaactcagaatgtatacagtgccaccccgaatgtcttcctcaggccatgaatatcacatgcaccggacgcggaccagacaactgtatccaatgtgctcactacattgacggacctcattgtgtgaaaacatgccccgcaggagttatgggagaaaacaacaccctcgtttggaaatatgccgatgcaggtcacgtatgtcacctgtgccacccaaactgcacttatgggtgcaccgggccgggcctggaggggtgccctacgaatggaccaaaaattcccagtattgcaactgggatggtcggggcactgttgttgctgcttgtggttgccctcgggataggcctgtttatgtctggctccggcgccaccaatttcagcctgctgaaacaggcaggcgacgtcgaagaaaatccaggaccaatgcgaatatcaaaaccacacttgcgcagcatttctatacagtgctatttgtgcttgttgctgaactctcacttcctcacagaggctgggatacacgttttcatacttggatgtttttcagctgggctgccgaagacagaggcgaattgggtgaatgtaatttcagacctcaagaagatcgaggatctcatccagtccatgcacatcgacgctactctgtacacagagagcgatgtccacccttcttgtaaggttaccgccatgaaatgcttccttttggaactccaagtcatctcattggaatcaggggatgcgtccattcatgacaccgtggaaaacctgataatactggctaacaacagcttgtcaagtaatgggaatgttactgagtccggttgtaaagaatgtgaagagctggaggagaagaacattaaggaatttttgcaatcttttgtacatattgttcagatgtttattaacacaagctgataaaacttgtttattgcagcttataatggttacaaataaagcaatagcatcacaaatttcacaaataaagcatttttttcactgcattctagttgtggtttgtccaaactcatcaatgtatcttatcatgtctgtaaggatcagccccaaggtaaaaaggccgggaaagcatcttaatttagcgtgcagtctcagctggtcctgccattccagataaacagagaaaccattctgaattggggatgggggtgaggatgggaacaggagtctgtgtcctgctggggcaggccattggaagatgtgaaagagttgtctatttccttccaccggagggagacttcaggtcagccaggtgtctggagtatgaaccatgtatcagcaccgaaaggttctagaagtcagactttcgggcagtgtgtcactaactctcagcatgctggcctggctcggcccacagcaaggtcttctcgcctccctttgggtaaatactgaggggtgcctctgcaggacgggacctctgccagactccactccatacccagagaagcagggaaaccaaaattggagtcagccttgaggtgtagctgttgagccctcagcagctggggagagctggcggatgctgccctccccccagtttcctaatggtgttgtttaaaaagggtcaggggacgggggaacagatggtgggaagagcacagtgcagacacctggcaccggctctgaaggcagcatggcagctacaccgttggctgggaagggtgtgcccctgaagaagtcgtttacattctcgagtcaattttcctggagtgtacaatggacctgtgggaaagcctgtatgaaagggtaatgatgagggacctagcacagtgtccaatattttataggaactggaattgagctcataggagctcaattttattggcattgctgttgttggatggttaaaggggtggtatcccttttctcagactcccctgaaatgtatggtttgctttgaacccagagactgatgacaggtctgccggtgtggttgggtgcagccttaagttgctacgggaaagtgttggagggggagaagtcagaggtaaccttgccccctccctcaattccagatgaggaaattcaggcctgaaaagggagatccaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattc

SEQ ID NO: 87 - DNA, 1042 bp, synthetic construct
  misc_feature 1..1042: note = B2M targeting left homology arm
  source 1..1042: mol_type = other DNA
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtc

SEQ ID NO: 88 - DNA, 1023 bp, synthetic construct
  misc_feature 1..1023: note = B2M targeting right homology arm
  source 1..1023: mol_type = other DNA
atccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcag

SEQ ID NO: 89 - DNA, 7813 bp, synthetic construct
  misc_feature 1..7813: note = B2M Targeting Plasmid
  source 1..7813: mol_type = other DNA
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcggcctccaacgcgtagatctattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 90 - DNA, 1205 bp, synthetic construct
  misc_feature 1..1205: note = AAVS1 targeting left homology arm
  source 1..1205: mol_type = other DNA
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccct

SEQ ID NO: 91 - DNA, 1200 bp, synthetic construct
  misc_feature 1..1200: note = AAVS1 targeting right homology arm
  source 1..1200: mol_type = other DNA
ccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctag

SEQ ID NO: 92 - DNA, 7971 bp, synthetic construct
  misc_feature 1..7971: note = AAVS1 Targeting plasmid
  source 1..7971: mol_type = other DNA
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctgcggccgcacgcgtagatctattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcacgacgactcctgctccaaggcctcctacacctgcaccaaccattgcaagtcagccgttgagcctccggccagaagcatgtcgcccagccgcaggcggggctgtacacacgagaggcttggatttcgcatgtgacatctatatctgggccccactggccggcacctgcggcgtgctgctgctgagcctggtgatcaccaagcgaggccgcaaaaaactcctttatatattcaagcaaccttttatgaggcccgtccagaccacgcaagaggaagatgggtgctcttgccgctttccagaggaagaggaggggggctgcgaacttagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataataaaacttgtttattgcagcttataatggttacaaataaagcaatagcatcacaaatttcacaaataaagcatttttttcactgcattctagttgtggtttgtccaaactcatcaatgtatcttaccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 93 - DNA, 24 bp, synthetic construct
  misc_feature 1..24: note = B2M target gRNA
  source 1..24: mol_type = other DNA
tttactcacgtcatccagcagaga

SEQ ID NO: 94 - DNA, 24 bp, synthetic construct
  misc_feature 1..24: note = AAVS1 target gRNA
  source 1..24: mol_type = other DNA
tttatctgtcccctccaccccaca

SEQ ID NO: 95 - DNA, 24 bp, synthetic construct
  misc_feature 1..24: note = CIITA target gRNA
  source 1..24: mol_type = other DNA
tttaccttggggctctgacaggta

SEQ ID NO: 96 - protein, 119 aa, synthetic construct
  REGION 1..119: note = IgG4 CH3 Hinge
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 21 aa, synthetic construct
  REGION 1..21: note = IgK Signal Peptide Variant
MARSPAQLLGLLLLWLSGARC

SEQ ID NO: 98 - DNA, 63 bp, synthetic construct
  misc_feature 1..63: note = IgK Signal Peptide Variant (nucleotide sequence)
  source 1..63: mol_type = other DNA
atggccagatctcctgctcaactgctgggactgctgctgctgtggcttagcggagccagatgc

SEQ ID NO: 99 - DNA, 54 bp, synthetic construct
  misc_feature 1..54: note = Whitlow Linker (nucleotide sequence)
  source 1..54: mol_type = other DNA
ggctctacaagcggcagcggcaaacctggatctggcgagggatctaccaagggc

SEQ ID NO: 100 - DNA, 321 bp, synthetic construct
  misc_feature 1..321: note = CD28 (nucleotide sequence)
  source 1..321: mol_type = other DNA
atcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtcc

SEQ ID NO: 101 - DNA, 336 bp, synthetic construct
  misc_feature 1..336: note = CD3-zeta isoform 3 (nucleotide sequence)
  source 1..336: mol_type = other DNA
agagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 102 - DNA, 54 bp, synthetic construct
  misc_feature 1..54: note = Whitlow Linker (nucleotide sequence)
  source 1..54: mol_type = other DNA
ggcagtacttctggtagcggaaaacccggtagcggcgaggggtcaactaaagga

SEQ ID NO: 103 - protein, 10 aa, synthetic construct
  REGION 1..10: note = Linker 35
GGGGSGGGGS

SEQ ID NO: 104 - protein, 5 aa, synthetic construct
  REGION 1..5: note = Linker 34
GGGGS

SEQ ID NO: 105 - protein, 122 aa, synthetic construct
EVQLLESGGGLVQAGGSLRLSCAASGSFFSIYAMGWFRQAPGKEREFVAAYISSGGLTSYADSVKGRFTISRDNAKNTVYLQMNSLKPEDTAVYYCAADLGAQTGYVQYDYWGQGTQVTVSS

SEQ ID NO: 106 - protein, 122 aa, synthetic construct
EVQLLESGGGLVQPGGSLRLSCAASGFVSSIYFMGWFRQAPGKEREFVSSSIGKGGSTRYADSVKGRFTISRDNSKNTLYLQMNSLKPEDTAVYYCAGDEGLGTAHAEYDYWGQGTQVTVSS

SEQ ID NO: 107 - protein, 122 aa, synthetic construct
EVQLLESGGGLVQAGGSLRLSCAASGGISEFYFMGWFRQAPGKEREFVAAEISPGSYTNYADSVKGRFTISRDNAKNTVYLQMNSLKPEDTAVYYCAADRDGDTYTAEYDYWGQGTQVTVSS

SEQ ID NO: 108 - protein, 117 aa, synthetic construct
EVQLLESGGGLVQPGGSLRLSCAASGSISSFYYIGWFRQAPGKEREFVSSRITSGGSTYYRDSVKGRFTISRDNSKNTLYLQMNSLKPEDTAVYYCAAGTSRDYYYWGQGTQVTVSS

SEQ ID NO: 109 - protein, 120 aa, synthetic construct
EVQLLESGGGLVQPGGSLRLSCAASGSTSSIGIMGWFRQAPGKERELVSSITAGGSTYYADSVKGRFTISRDNSKNTLYLQMNSLKPEDTAVYYCNAHVGYGRVHDVDYWGQGTQVTVSS

SEQ ID NO: 110 - protein, 117 aa, synthetic construct
EVQLLESGGGLVQPGGSLRLSCAASGFVSPSYIMGWFRQAPGKEREFVSSVIEYRGSTYYLDSVKGRFTISRDNSKNTLYLQMNSLKPEDTAVYYCAAGTPGGYDYWGQGTQVTVSS

SEQ ID NO: 111 - protein, 117 aa, synthetic construct
EVQLLESGGGLVQPGGSLRLSCAASGSTFSSNAMGWYRQAPGKERELVSSISGSGGSTRYADSVKGRFTISRDNSKNTLYLQMNSLKPEDTAVYYCASYVLYLREYWGQGTQVTVSS

SEQ ID NO: 112 - protein, 115 aa, synthetic construct
EVQLLESGGGLVQPGGSLRLSCAASGLTFRYNAMGWYRQAPGKEREFVSAISGSGGGTYYADSVKGRFTISRDNSKNTLYLQMNSLKPEDTAVYYCAAEGLYDYWGQGTQVTVSS

SEQ ID NO: 113 - protein, 118 aa, synthetic construct
EVQLLESGGGLVQPGGSLRLSCAASGLTSSGYAMGWYRQAPGKERELVSSISSSGGLTHYADSVKGRFTISRDNSKNTLYLQMNSLKPEDTAVYYCDADIAYTGADYWGQGTQVTVSS

SEQ ID NO: 114 - protein, 115 aa, synthetic construct
EVQLLESGGGLVQPGGSLRLSCAASGFTFSDYYMGWYRQAPGKEREFVSAISSTGGSPYYADSVKGRFTISRDNSKNTLYLQMNSLKPEDTAVYYCAVEVPYDYWGQGTQVTVSS

SEQ ID NO: 115 - protein, 116 aa, synthetic construct
EVQLLESGGGLVQPGGSLRLSCAASGFTYSSYAMGWYRQAPGKERELVSSISGSGGSTRYADSVKGRFTISRDNSKNTLYLQMNSLKPEDTAVYYCAVAIGVGDYWGQGTQVTVSS

SEQ ID NO: 116 - protein, 115 aa, synthetic construct
EVQLLESGGGLVQPGGSLRLSCAASGFTLSSYAMGWYRQAPGKERELVSSISGSGGSTRYADSVKGRFTISRDNSKNTLYLQMNSLKPEDTAVYYCAAYIGGDYLGQGTQVTVSS

SEQ ID NO: 117 - protein, 116 aa, synthetic construct
EVQLLESGGGLVQPGGSLRLSCAASGLTLSSYAMGWFRQAPGKERELVSSISGSGGSTRYADSVKGRFTISRDNSKNTLYLQMNSLKPEDTAVYYCAVDIRLTDYWGQGTQVTVSS

SEQ ID NO: 118 - protein, 115 aa, synthetic construct
EVQLLESGGGLVQPGGSLRLSCAASGFAYSSYYMGWYRQAPGKEREFVSAISSSGGGTYYADSVKGRFTISRDNSKNTLYLQMNSLKPEDTAVYYCAVELTYNYWGQGTQVTVSS

SEQ ID NO: 119 - protein, 119 aa, synthetic construct
EVQLLESGGGLVQPGGSLRLSCAASGFTFRSYAMGWFRQAPGKEREGVSVITGTGGSTYYADSVKGRFTISRDNSKNTLYLQMNSLKPEDTAVYYCARAYLSGYLYEYWGQGTQVTVSS

SEQ ID NO: 120 - protein, 116 aa, synthetic construct; identical to SEQ ID NO: 117

SEQ ID NO: 121 - protein, 122 aa, synthetic construct
EVQLLESGGGLVQPGGSLRLSCAASGLSFSSYSMGWYRQAPGKEREFVSAISGSSGSTNYADSVKGRFTISRDNSKNTLYLQMNSLKPEDTAVYYCAAEHRVTTSGVFYDYWGQGTQVTVSS

SEQ ID NO: 122 - protein, 120 aa, synthetic construct
EVQLLESGGGLVQPGGSLRLSCAASGFTFSSYAMGWYRQAPGKEREFVSSISGSGGLTRYADSVKGRFTISRDNSKNTLYLQMNSLKPEDTAVYYCAVTTGYQGGVYDYWGQGTQVTVSS

SEQ ID NO: 123 - protein, 122 aa, synthetic construct
EVQLLESGGGLVQPGGSLRLSCAASGFTYSGYAMGWYRQAPGKERELVSSISGSGTLTSYADSVKGRFTISRDNSKNTLYLQMNSLKPEDTAVYYCDVDIPVGDATTVGDYWGQGTQVTVSS

SEQ ID NO: 124 - protein, 120 aa, synthetic construct
EVQLLESGGGLVQPGGSLRLSCAASGLTFSSNAMGWYRQAPGKERELVSSISGSGGSTNYADSVKGRFTISRDNSKNTLYLQMNSLKPEDTAVYYCAAEIATYYDFADYWGQGTQVTVSS

SEQ ID NO: 125 - protein, 121 aa, synthetic construct
EVQLLESGGGLVQPGGSLRLSCAASGFTYTSYVMGWYRQAPGKERELVSAIYGSAGSGYYADSVKGRFTISRDNSKNTLYLQMNSLKPEDTAVYYCARVTTTTHLWSAEYWGQGTQVTVSS

SEQ ID NO: 126 - protein, 120 aa, synthetic construct
EVQLLESGGGLVQPGGSLRLSCAASGRTLSSYAMGWYRQAPGKERELVSSISGSGGSTRYADSVKGRFTISRDNSKNTLYLQMNSLKPEDTAVYYCAVYILELAPGAEYWGQGTQVTVSS

SEQ ID NO: 127 - protein, 121 aa, synthetic construct
EVQLLESGGGLVQPGGSLRLSCAASGYTFSDYAMGWYRQAPGKERELVSSISGSGGSTRYADSVKGRFTISRDNSKNTLYLQMNSLKPEDTAVYYCAAVIRQPSTGFYEYWGQGTQVTVSS

SEQ ID NO: 128 - protein, 121 aa, synthetic construct
EVQLLESGGGLVQPGGSLRLSCAASGFAYSSYSIGWYRQAPGKERELVSAISGSGGSSRYADSVKGRFTISRDNSKNTLYLQMNSLKPEDTAVYYCAAIAYVYTSTARNYWGQGTQVTVSS

SEQ ID NO: 129 - protein, 120 aa, synthetic construct
EVQLLESGGGLVQPGGSLRLSCAASGFTYSDYAMGWYRQAPGKERELVSAISSYGGVTNYADSVKGRFTISRDNSKNTLYLQMNSLKPEDTAVYYCAAYGALSIRGYDYWGQGTQVTVSS

SEQ ID NO: 130 - protein, 121 aa, synthetic construct
EVQLLESGGGLVQPGGSLRLSCAASGFAFSIDAMGWYRQAPGKEREGVSSISGSGGLTRYADSVKGRFTISRDNSKNTLYLQMNSLKPEDTAVYYCDTAVLTGYSEAFDYWGQGTQVTVSS

SEQ ID NO: 131 - DNA, 366 bp, synthetic construct
  source 1..366: mol_type = other DNA
gaggtacaacttttggagtcaggcggcgggttggtccaggcgggtggctcactccgccttagttgtgccgcctcagggtcattctttagtatctacgctatgggctggtttcgacaggcccctggtaaggaacgtgagtttgtggccgcctacatttcctcaggggggctcaccagctacgcggatagtgttaagggtagattcaccatctccagagacaatgcaaagaatacggtatacctccaaatgaacagcctgaagcctgaagacacggctgtctactattgcgcagcagacttgggagcccagaccggatacgttcagtacgactactgggggcagggaacccaggtgacggtctcgagc

SEQ ID NO: 132 - DNA, 366 bp, synthetic construct
  source 1..366: mol_type = other DNA
gaggtacaacttttggagtcaggcggcggcctggtgcagcccggagggagtctccgactgtcttgcgctgcatctggattcgtgagctctatatactttatgggatggttcaggcaggctcctgggaaggagcgcgagtttgtgtctagtagtattggcaagggtggctcaacacgctatgcggattctgtgaaagggaggttcacaataagcagggacaactcaaagaatacactgtacctccagatgaactccttaaaaccagaggatactgcagtctattactgtgctggagatgagggattgggaactgcacatgctgaatacgactactggggccaggggacccaggtgacggtctcgagc

SEQ ID NO: 133 - DNA, 366 bp, synthetic construct
  source 1..366: mol_type = other DNA
gaggtacaacttttggagtcaggcggaggactcgtgcaggctggagggtcccttaggctcagctgcgccgcttccggagggatctccgaattctatttcatggggtggtttagacaagcgcccggaaaggaaagggaattcgtcgcagcagagatctcacctggaagctacaccaactacgcagatagcgtgaaggggcgctttaccatctctagggataacgcgaaaaatacagtctacctccaaatgaactctttgaagccagaggataccgccgtgtattattgcgctgccgacagggacggagacacatatacagcagaatatgattattggggccaggggacccaggtgacggtctcgagc

SEQ ID NO: 134 - DNA, 351 bp, synthetic construct
  source 1..351: mol_type = other DNA
gaggtacaacttttggagtcaggcggcgggttagtgcagcctggaggatcactgaggctgagctgcgccgcctctggctcaattagcagtttctattatatcggatggttccgccaggctccgggaaaagagagagagtttgtttcctctcgcattacctcaggaggaagcacttactacagggactctgttaaaggacgctttacaatctccagagataattccaagaacaccttatatctgcaaatgaatagtttgaagcccgaggacactgccgtgtattattgcgcagccgggacatcccgcgattactactactggggacaaggaacccaggtgacggtctcgagc

SEQ ID NO: 135 - DNA, 360 bp, synthetic construct
  source 1..360: mol_type = other DNA
gaggtacaacttttggagtcaggcggggggctggtgcagcctgggggatcactgcgcctttcatgcgcagcgagtggttccacctctagtatcggaattatgggctggtttcgccaggctcctggaaaggaaagggagctggtctccagcatcacagccggcggatctacctactacgccgactccgttaaggggcgattcactatctcccgcgacaatagcaagaacaccttgtatctgcagatgaactccctcaaacccgaggatactgccgtgtactattgcaacgcacatgtgggctacgggagggtgcacgatgtggattactgggggcaggggacccaggtgacggtctcgagc

SEQ ID NO: 136 - DNA, 351 bp, synthetic construct
  source 1..351: mol_type = other DNA
gaggtacaacttttggagtcaggcggtggtctcgtccagccgggggggagcctccgcttatcatgtgccgcctccggatttgtctcaccctcttatataatggggtggttccggcaagcaccaggcaaagaacgggagtttgtttcctccgtcatagagtaccgagggtccacctattacctggatagcgtgaaggggcggttcaccatctcccgcgataattccaagaataccctgtacctgcagatgaatagtctgaaacctgaggacacggccgtgtactattgcgcagcgggcacccccggaggctacgattactggggccaagggacccaggtgacggtctcgagc

SEQ ID NO: 137 - DNA, 351 bp, synthetic construct
  source 1..351: mol_type = other DNA
gaggtacaacttttggagtcaggcggtggactggtacaaccgggtggttcattgcgtttgagctgcgctgcctctggttctaccttctcaagcaatgcaatgggttggtatcgccaagcgccgggcaaagaacgcgagttggtctcctccatatccggttccggtggtagcacccgctacgcggactcggtaaaaggccgttttacgatcagtcgtgataattccaagaataccttgtacctgcaaatgaatagccttaagcccgaagacacagcggtgtattattgtgcctcttatgtcctgtatctccgagaatactggggccagggtacccaggtgacggtctcgagc

SEQ ID NO: 138 - DNA, 345 bp, synthetic construct
  source 1..345: mol_type = other DNA
gaggtacaacttttggagtcaggcggtggactggtacaaccgggtggttcattgcgtttgagctgcgctgcctctggtctgacttttaggtataacgcaatgggatggtaccgccaagcgccgggcaaagaacgcgagtttgtctccgcgatcagcggctctggggggggtacctattacgcggactcggtaaaaggccgttttacgatcagtcgtgataattccaagaataccttgtacctgcaaatgaatagccttaagcccgaagacacagcggtgtattattgtgccgcggaaggcctgtatgattattggggccagggtacccaggtgacggtctcgagc

SEQ ID NO: 139 - DNA, 354 bp, synthetic construct
  source 1..354: mol_type = other DNA
gaggtacaacttttggagtcaggcggtggactggtacaaccgggtggttcattgcgtttgagctgcgctgcctctggtttaacaagctctggttacgctatgggctggtatcgccaagcgccgggcaaagaacgcgagctggtgagcagtatttcttcctcaggcggactgacccattacgcggactcggtaaaaggccgttttacgatcagtcgtgataattccaagaataccttgtacctgcaaatgaatagccttaagcccgaagacacagcggtgtattattgtgacgcagatattgcttacactggcgccgattattggggccagggtacccaggtgacggtctcgagc

SEQ ID NO: 140 - DNA, 345 bp, synthetic construct
  source 1..345: mol_type = other DNA
gaggtacaacttttggagtcaggcggtggactggtacaaccgggtggttcattgcgtttgagctgcgctgcctctggttttacgtttagcgactattatatgggctggtaccgccaagcgccgggcaaagaacgcgagttcgtatcagctatttcgtccacgggcggctccccctactacgcggactcggtaaaaggccgttttacgatcagtcgtgataattccaagaataccttgtacctgcaaatgaatagccttaagcccgaagacacagcggtgtattattgtgccgtggaagtcccttacgattattggggccagggtacccaggtgacggtctcgagc

SEQ ID NO: 141 - DNA, 348 bp, synthetic construct
  source 1..348: mol_type = other DNA
gaggtacaacttttggagtcaggcggtggactggtacaaccgggtggttcattgcgtttgagctgcgctgcctctggttttacgtactcttcttatgcaatgggctggtatcgccaagcgccgggcaaagaacgcgagttggtctcctccatatccggttccggtggtagcacccgctacgcggactcggtaaaaggccgttttacgatcagtcgtgataattccaagaataccttgtacctgcaaatgaatagccttaagcccgaagacacagcggtgtattattgtgccgtggcgattggcgtaggagactattggggccagggtacccaggtgacggtctcgagc

SEQ ID NO: 142 - DNA, 345 bp, synthetic construct
  source 1..345: mol_type = other DNA
gaggtacaacttttggagtcaggcggtggactggtacaaccgggtggttcattgcgtttgagctgcgctgcctctggttttaccctgtcttcgtatgcaatgggctggtaccgccaagcgccgggcaaagaacgcgagctcgtttccagcatcagtgggtcgggtggctctacccggtacgcggactcggtaaaaggccgttttacgatcagtcgtgataattccaagaataccttgtacctgcaaatgaatagccttaagcccgaagacacagcggtgtattattgtgccgcttacatcggcggagattacttgggccagggtacccaggtgacggtctcgagc

SEQ ID NO: 143 - DNA, 348 bp, synthetic construct
  source 1..348: mol_type = other DNA
gaggtacaacttttggagtcaggcggtggactggtacaaccgggtggttcattgcgtttgagctgcgctgcctctggtctgacactgtcatcgtacgcaatgggttggttccgccaagcgccgggcaaagaacgcgagctggtgtcctcaataagcggtagtggcggcagcacgcgctacgcggactcggtaaaaggccgttttacgatcagtcgtgataattccaagaataccttgtacctgcaaatgaatagccttaagcccgaagacacagcggtgtattattgtgccgttgatatccggctgacggattattggggccagggtacccaggtgacggtctcgagc

SEQ ID NO: 144 - DNA, 345 bp, synthetic construct
  source 1..345: mol_type = other DNA
gaggtacaacttttggagtcaggcggtggactggtacaaccgggtggttcattgcgtttgagctgcgctgcctctggttttgcctactcttcgtattacatgggttggtatcgccaagcgccgggcaaagaacgcgagtttgtgagtgcaatatcatctagtggcggcggtacctactacgcggactcggtaaaaggccgttttacgatcagtcgtgataattccaagaataccttgtacctgcaaatgaatagccttaagcccgaagacacagcggtgtattattgtgccgttgaacttacctataactactggggccagggtacccaggtgacggtctcgagc

SEQ ID NO: 145 - DNA, 348 bp, synthetic construct
  source 1..348: mol_type = other DNA
gaggtacaacttttggagtcaggcggtggactggtacaaccgggtggttcattgcgtttgagctgcgctgcctctggtttcatatactctagctactatatgggatggtaccgccaagcgccgggcaaagaacgcgagtttgttagcgctattacctcatcaggcggttctacatattacgcggactcggtaaaaggccgttttacgatcagtcgtgataattccaagaataccttgtacctgcaaatgaatagccttaagcccgaagacacagcggtgtattattgtgccgtggaagtccctgaatttgattattggggccagggtacccaggtgacggtctcgagc

SEQ ID NO: 146 - DNA, 357 bp, synthetic construct
  source 1..357: mol_type = other DNA
gaggtacaacttttggagtcaggcggtggactggtacaaccgggtggttcattgcgtttgagctgcgctgcctctggtttcacatttcgttcttatgcgatggggtggtttcgccaagcgccgggcaaagaacgcgagggtgtttctgtgattacaggtactggcggttcaacctattacgcggactcggtaaaaggccgttttacgatcagtcgtgataattccaagaataccttgtacctgcaaatgaatagccttaagcccgaagacacagcggtgtattattgtgcccgcgcatatctgagtggctatctgtacgagtattggggccagggtacccaggtgacggtctcgagc

SEQ ID NO: 147 - DNA, 366 bp, synthetic construct
  source 1..366: mol_type = other DNA
gaggtacaacttttggagtcaggcggtggactggtacaaccgggtggttcattgcgtttgagctgcgctgcctctggtctgagcttctcatcttattcaatggggtggtatcgccaagcgccgggcaaagaacgcgagtttgtttctgcaatctcaggctcatcaggttcaacgaattacgcggactcggtaaaaggccgttttacgatcagtcgtgataattccaagaataccttgtacctgcaaatgaatagccttaagcccgaagacacagcggtgtattattgtgccgccgaacaccgggtgaccacttccggagttttctacgactattggggccagggtacccaggtgacggtctcgagc

SEQ ID NO: 148 - DNA, 360 bp, synthetic construct
  source 1..360: mol_type = other DNA
gaggtacaacttttggagtcaggcggtggactggtacaaccgggtggttcattgcgtttgagctgcgctgcctctggtttcactttcagcagttatgcgatgggatggtatcgccaagcgccgggcaaagaacgcgagtttgttagcagcataagcggttcaggggggttaacccggtacgcggactcggtaaaaggccgttttacgatcagtcgtgataattccaagaataccttgtacctgcaaatgaatagccttaagcccgaagacacagcggtgtattattgtgccgtaaccaccggctaccaaggcggcgtatatgactactggggccagggtacccaggtgacggtctcgagc

SEQ ID NO: 149 - DNA, 366 bp, synthetic construct
  source 1..366: mol_type = other DNA
gaggtacaacttttggagtcaggcggtggactggtacaaccgggtggttcattgcgtttgagctgcgctgcctctggtttcacttactctgggtatgcaatggggtggtatcgccaagcgccgggcaaagaacgcgagctagtctcaagcatctcaggttcgggtacccttacttcgtacgcggactcggtaaaaggccgttttacgatcagtcgtgataattccaagaataccttgtacctgcaaatgaatagccttaagcccgaagacacagcggtgtattattgtgacgtagatatacctgtgggcgacgctacaaccgttggtgattactggggccagggtacccaggtgacggtctcgagc

SEQ ID NO: 150 - DNA, 360 bp, synthetic construct
  source 1..360: mol_type = other DNA
gaggtacaacttttggagtcaggcggtggactggtacaaccgggtggttcattgcgtttgagctgcgctgcctctggtcttacattttctagcaatgctatgggttggtaccgccaagcgccgggcaaagaacgcgagctggtgagttctatttcggggagcggaggtagtaccaattacgcggactcggtaaaaggccgttttacgatcagtcgtgataattccaagaataccttgtacctgcaaatgaatagccttaagcccgaagacacagcggtgtattattgtgccgcggagattgctacctactacgattttgcagattactggggccagggtacccaggtgacggtctcgagc

SEQ ID NO: 151 - DNA, 363 bp, synthetic construct
  source 1..363: mol_type = other DNA
gaggtacaacttttggagtcaggcggtggactggtacaaccgggtggttcattgcgtttgagctgcgctgcctctggttttacctatacgtcttacgtcatgggctggtatcgccaagcgccgggcaaagaacgcgagctggtatctgcgatctatggcagtgcaggcagtgggtactacgcggactcggtaaaaggccgttttacgatcagtcgtgataattccaagaataccttgtacctgcaaatgaatagccttaagcccgaagacacagcggtgtattattgtgcccgcgttactaccacaacccacctgtggtctgccgagtattggggccagggtacccaggtgacggtctcgagc

SEQ ID NO: 152 - DNA, 360 bp, synthetic construct
  source 1..360: mol_type = other DNA
gaggtacaacttttggagtcaggcggtggactggtacaaccgggtggttcattgcgtttgagctgcgctgcctctggtcgtacgctaagtagttacgcgatggggtggtaccgccaagcgccgggcaaagaacgcgagctcgttagtagtatttccgggagtggaggctctacccgttacgcggactcggtaaaaggccgttttacgatcagtcgtgataattccaagaataccttgtacctgcaaatgaatagccttaagcccgaagacacagcggtgtattattgtgccgtctacattctggagctggctcctggcgcggaatactggggccagggtacccaggtgacggtctcgagc

SEQ ID NO: 153 - DNA, 363 bp, synthetic construct
  source 1..363: mol_type = other DNA
gaggtacaacttttggagtcaggcggtggactggtacaaccgggtggttcattgcgtttgagctgcgctgcctctggttacacattttcggattatgcaatgggttggtatcgccaagcgccgggcaaagaacgcgagctcgttagtagtatttccgggagtggaggctctacccgttacgcggactcggtaaaaggccgttttacgatcagtcgtgataattccaagaataccttgtacctgcaaatgaatagccttaagcccgaagacacagcggtgtattattgtgccgcagtaattcggcagcctagcaccggtttctatgaatactggggccagggtacccaggtgacggtctcgagc

SEQ ID NO: 154 - DNA, 363 bp, synthetic construct
  source 1..363: mol_type = other DNA
gaggtacaacttttggagtcaggcggtggactggtacaaccgggtggttcattgcgtttgagctgcgctgcctctggttttgcatattcgagctacagcatcggctggtaccgccaagcgccgggcaaagaacgcgagttagtcagcgctatctccggctctggaggctcctcgcgctacgcggactcggtaaaaggccgttttacgatcagtcgtgataattccaagaataccttgtacctgcaaatgaatagccttaagcccgaagacacagcggtgtattattgtgccgccatagcgtacgtgtacacgtccacggcccgtaattactggggccagggtacccaggtgacggtctcgagc

SEQ ID NO: 155 - DNA, 360 bp, synthetic construct
  source 1..360: mol_type = other DNA
gaggtacaacttttggagtcaggcggtggactggtacaaccgggtggttcattgcgtttgagctgcgctgcctctggtttcacctatagcgattatgcgatgggatggtaccgccaagcgccgggcaaagaacgcgagttggtttcagcgatctcatcgtacgggggcgttacaaattacgctgactcggtaaaaggccgttttacgatcagtcgtgataattccaagaataccttgtacctgcaaatgaatagccttaagcccgaagacacagcggtgtattattgtgccgcctatggcgctctgtccattcggggctatgattactggggccagggtacccaggtgacggtctcgagc

SEQ ID NO: 156 - DNA, 363 bp, synthetic construct
  source 1..363: mol_type = other DNA
gaggtacaacttttggagtcaggcggtggactggtacaaccgggtggttcattgcgtttgagctgcgctgcctctggttttgcgttctccatcgacgcgatgggttggtatcgccaagcgccgggcaaagaacgcgagggcgtttcctccatatcgggtagcggtggtctgacacgctacgcggactcggtaaaaggccgttttacgatcagtcgtgataattccaagaataccttgtacctgcaaatgaatagccttaagcccgaagacacagcggtgtattattgtgacacagccgtactgactggctattccgaggcttttgactactggggccagggtacccaggtgacggtctcgagc

SEQ ID NO: 157 - protein, 369 aa, synthetic construct
MARSPAQLLGLLLLWLSGARCEVQLLESGGGLVQAGGSLRLSCAASGSFFSIYAMGWFRQAPGKEREFVAAYISSGGLTSYADSVKGRFTISRDNAKNTVYLQMNSLKPEDTAVYYCAADLGAQTGYVQYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 369 aa, synthetic construct
MARSPAQLLGLLLLWLSGARCEVQLLESGGGLVQPGGSLRLSCAASGFVSSIYFMGWFRQAPGKEREFVSSSIGKGGSTRYADSVKGRFTISRDNSKNTLYLQMNSLKPEDTAVYYCAGDEGLGTAHAEYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 369 aa, synthetic construct
MARSPAQLLGLLLLWLSGARCEVQLLESGGGLVQAGGSLRLSCAASGGISEFYFMGWFRQAPGKEREFVAAEISPGSYTNYADSVKGRFTISRDNAKNTVYLQMNSLKPEDTAVYYCAADRDGDTYTAEYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 364 aa, synthetic construct
MARSPAQLLGLLLLWLSGARCEVQLLESGGGLVQPGGSLRLSCAASGSISSFYYIGWFRQAPGKEREFVSSRITSGGSTYYRDSVKGRFTISRDNSKNTLYLQMNSLKPEDTAVYYCAAGTSRDYYYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 367 aa, synthetic construct
MARSPAQLLGLLLLWLSGARCEVQLLESGGGLVQPGGSLRLSCAASGSTSSIGIMGWFRQAPGKERELVSSITAGGSTYYADSVKGRFTISRDNSKNTLYLQMNSLKPEDTAVYYCNAHVGYGRVHDVDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 364 aa, synthetic construct
MARSPAQLLGLLLLWLSGARCEVQLLESGGGLVQPGGSLRLSCAASGFVSPSYIMGWFRQAPGKEREFVSSVIEYRGSTYYLDSVKGRFTISRDNSKNTLYLQMNSLKPEDTAVYYCAAGTPGGYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 365 aa, synthetic construct
MARSPAQLLGLLLLWLSGARCEVQLLESGGGLVQPGGSLRLSCAASGLTSSGYAMGWYRQAPGKERELVSSISSSGGLTHYADSVKGRFTISRDNSKNTLYLQMNSLKPEDTAVYYCDADIAYTGADYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 363 aa, synthetic construct
MARSPAQLLGLLLLWLSGARCEVQLLESGGGLVQPGGSLRLSCAASGFTYSSYAMGWYRQAPGKERELVSSISGSGGSTRYADSVKGRFTISRDNSKNTLYLQMNSLKPEDTAVYYCAVAIGVGDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 363 aa, synthetic construct; identical to SEQ ID NO: 164

SEQ ID NO: 166 - protein, 369 aa, synthetic construct
MARSPAQLLGLLLLWLSGARCEVQLLESGGGLVQPGGSLRLSCAASGLSFSSYSMGWYRQAPGKEREFVSAISGSSGSTNYADSVKGRFTISRDNSKNTLYLQMNSLKPEDTAVYYCAAEHRVTTSGVFYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 367 aa, synthetic construct
MARSPAQLLGLLLLWLSGARCEVQLLESGGGLVQPGGSLRLSCAASGFTFSSYAMGWYRQAPGKEREFVSSISGSGGLTRYADSVKGRFTISRDNSKNTLYLQMNSLKPEDTAVYYCAVTTGYQGGVYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 369 aa, synthetic construct
MARSPAQLLGLLLLWLSGARCEVQLLESGGGLVQPGGSLRLSCAASGFTYSGYAMGWYRQAPGKERELVSSISGSGTLTSYADSVKGRFTISRDNSKNTLYLQMNSLKPEDTAVYYCDVDIPVGDATTVGDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 367 aa, synthetic construct
MARSPAQLLGLLLLWLSGARCEVQLLESGGGLVQPGGSLRLSCAASGRTLSSYAMGWYRQAPGKERELVSSISGSGGSTRYADSVKGRFTISRDNSKNTLYLQMNSLKPEDTAVYYCAVYILELAPGAEYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 368 aa, synthetic construct
MARSPAQLLGLLLLWLSGARCEVQLLESGGGLVQPGGSLRLSCAASGYTFSDYAMGWYRQAPGKERELVSSISGSGGSTRYADSVKGRFTISRDNSKNTLYLQMNSLKPEDTAVYYCAAVIRQPSTGFYEYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - DNA, 1107 bp, synthetic construct
  source 1..1107: mol_type = other DNA
atggccagatctcctgctcaactgctgggactgctgctgctgtggcttagcggagccagatgcgaggtacaacttttggagtcaggcggcgggttggtccaggcgggtggctcactccgccttagttgtgccgcctcagggtcattctttagtatctacgctatgggctggtttcgacaggcccctggtaaggaacgtgagtttgtggccgcctacatttcctcaggggggctcaccagctacgcggatagtgttaagggtagattcaccatctccagagacaatgcaaagaatacggtatacctccaaatgaacagcctgaagcctgaagacacggctgtctactattgcgcagcagacttgggagcccagaccggatacgttcagtacgactactgggggcagggaacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 172 - DNA, 1107 bp, synthetic construct
  source 1..1107: mol_type = other DNA
atggccagatctcctgctcaactgctgggactgctgctgctgtggcttagcggagccagatgcgaggtacaacttttggagtcaggcggcggcctggtgcagcccggagggagtctccgactgtcttgcgctgcatctggattcgtgagctctatatactttatgggatggttcaggcaggctcctgggaaggagcgcgagtttgtgtctagtagtattggcaagggtggctcaacacgctatgcggattctgtgaaagggaggttcacaataagcagggacaactcaaagaatacactgtacctccagatgaactccttaaaaccagaggatactgcagtctattactgtgctggagatgagggattgggaactgcacatgctgaatacgactactggggccaggggacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 173 - DNA, 1107 bp, synthetic construct; identical to SEQ ID NO: 171
  source 1..1107: mol_type = other DNA

SEQ ID NO: 174 - DNA, 1092 bp, synthetic construct
  source 1..1092: mol_type = other DNA
atggccagatctcctgctcaactgctgggactgctgctgctgtggcttagcggagccagatgcgaggtacaacttttggagtcaggcggcgggttagtgcagcctggaggatcactgaggctgagctgcgccgcctctggctcaattagcagtttctattatatcggatggttccgccaggctccgggaaaagagagagagtttgtttcctctcgcattacctcaggaggaagcacttactacagggactctgttaaaggacgctttacaatctccagagataattccaagaacaccttatatctgcaaatgaatagtttgaagcccgaggacactgccgtgtattattgcgcagccgggacatcccgcgattactactactggggacaaggaacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 175 - DNA, 1101 bp, synthetic construct
  source 1..1101: mol_type = other DNA
atggccagatctcctgctcaactgctgggactgctgctgctgtggcttagcggagccagatgcgaggtacaacttttggagtcaggcggggggctggtgcagcctgggggatcactgcgcctttcatgcgcagcgagtggttccacctctagtatcggaattatgggctggtttcgccaggctcctggaaaggaaagggagctggtctccagcatcacagccggcggatctacctactacgccgactccgttaaggggcgattcactatctcccgcgacaatagcaagaacaccttgtatctgcagatgaactccctcaaacccgaggatactgccgtgtactattgcaacgcacatgtgggctacgggagggtgcacgatgtggattactgggggcaggggacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 176 - DNA, 1092 bp, synthetic construct
  source 1..1092: mol_type = other DNA
atggccagatctcctgctcaactgctgggactgctgctgctgtggcttagcggagccagatgcgaggtacaacttttggagtcaggcggtggtctcgtccagccgggggggagcctccgcttatcatgtgccgcctccggatttgtctcaccctcttatataatggggtggttccggcaagcaccaggcaaagaacgggagtttgtttcctccgtcatagagtaccgagggtccacctattacctggatagcgtgaaggggcggttcaccatctcccgcgataattccaagaataccctgtacctgcagatgaatagtctgaaacctgaggacacggccgtgtactattgcgcagcgggcacccccggaggctacgattactggggccaagggacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 177 - DNA, 1095 bp, synthetic construct
  source 1..1095: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggtttaacaagctctggttacgctatgggctggtatcgccaagcgccgggcaaagaacgcgagctggtgagcagtatttcttcctcaggcggactgacccattacgcggactcggtaaaaggccgttttacgatcagtcgtgataattccaagaataccttgtacctgcaaatgaatagccttaagcccgaagacacagcggtgtattattgtgacgcagatattgcttacactggcgccgattattggggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 178 - DNA, 1089 bp, synthetic construct
  source 1..1089: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggttttacgtactcttcttatgcaatgggctggtatcgccaagcgccgggcaaagaacgcgagttggtctcctccatatccggttccggtggtagcacccgctacgcggactcggtaaaaggccgttttacgatcagtcgtgataattccaagaataccttgtacctgcaaatgaatagccttaagcccgaagacacagcggtgtattattgtgccgtggcgattggcgtaggagactattggggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 179 - DNA, 1086 bp, synthetic construct
  source 1..1086: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggttttaccctgtcttcgtatgcaatgggctggtaccgccaagcgccgggcaaagaacgcgagctcgtttccagcatcagtgggtcgggtggctctacccggtacgcggactcggtaaaaggccgttttacgatcagtcgtgataattccaagaataccttgtacctgcaaatgaatagccttaagcccgaagacacagcggtgtattattgtgccgcttacatcggcggagattacttgggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 180 - DNA, 1107 bp, synthetic construct
  source 1..1107: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggtctgagcttctcatcttattcaatggggtggtatcgccaagcgccgggcaaagaacgcgagtttgtttctgcaatctcaggctcatcaggttcaacgaattacgcggactcggtaaaaggccgttttacgatcagtcgtgataattccaagaataccttgtacctgcaaatgaatagccttaagcccgaagacacagcggtgtattattgtgccgccgaacaccgggtgaccacttccggagttttctacgactattggggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 181 - DNA, 1101 bp, synthetic construct
  source 1..1101: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggtttcactttcagcagttatgcgatgggatggtatcgccaagcgccgggcaaagaacgcgagtttgttagcagcataagcggttcaggggggttaacccggtacgcggactcggtaaaaggccgttttacgatcagtcgtgataattccaagaataccttgtacctgcaaatgaatagccttaagcccgaagacacagcggtgtattattgtgccgtaaccaccggctaccaaggcggcgtatatgactactggggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 182 - DNA, 1107 bp, synthetic construct
  source 1..1107: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggtttcacttactctgggtatgcaatggggtggtatcgccaagcgccgggcaaagaacgcgagctagtctcaagcatctcaggttcgggtacccttacttcgtacgcggactcggtaaaaggccgttttacgatcagtcgtgataattccaagaataccttgtacctgcaaatgaatagccttaagcccgaagacacagcggtgtattattgtgacgtagatatacctgtgggcgacgctacaaccgttggtgattactggggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 183 - DNA, 1101 bp, synthetic construct
  source 1..1101: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggtcgtacgctaagtagttacgcgatggggtggtaccgccaagcgccgggcaaagaacgcgagctcgttagtagtatttccgggagtggaggctctacccgttacgcggactcggtaaaaggccgttttacgatcagtcgtgataattccaagaataccttgtacctgcaaatgaatagccttaagcccgaagacacagcggtgtattattgtgccgtctacattctggagctggctcctggcgcggaatactggggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 184 - DNA, 1104 bp, synthetic construct
  source 1..1104: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggttacacattttcggattatgcaatgggttggtatcgccaagcgccgggcaaagaacgcgagctcgttagtagtatttccgggagtggaggctctacccgttacgcggactcggtaaaaggccgttttacgatcagtcgtgataattccaagaataccttgtacctgcaaatgaatagccttaagcccgaagacacagcggtgtattattgtgccgcagtaattcggcagcctagcaccggtttctatgaatactggggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 185 - DNA, 1479 bp, synthetic construct
  source 1..1479: mol_type = other DNA
atgtggcaactgttgctgcctacagctctgctgctgctggtgtctgccggcatgagaacagaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagaaagtacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctatcaggtgtccttctgcctggtcatggtgctgctgttcgccgtggataccggcctgtacttcagcgtcaagaccaacatccggtccagcaccagagactggaaggaccacaagttcaagtggcggaaggaccctcaggacaaaggcagcggcgccaccaatttcagcctgctgaaacaggctggcgacgtggaagagaaccctggacctatgggctggatccggggcagaagaagcagacacagctgggagatgagcgagttccacaattacaacctggacctgaagaagtccgacttcagcacccggtggcagaaacagagatgccccgtggtcaagagcaagtgcagagagaacgctagccccttcttcttctgttgctttatcgccgtggccatgggcatccgcttcatcatcatggtcacaatttggagcgccgtgttcctgaactccctgttcaatcaagaggtgcagatccctctgaccgagagctactgtggcccctgtcctaagaactggatctgctacaagaacaactgctaccagttcttcgacgagagcaagaattggtacgagagccaggcctcctgcatgagccagaatgcttccctgctgaaggtgtacagcaaagaggaccaggatctgctgaagctggtcaagtcctaccactggatgggcctcgtgcacatccctaccaatggctcttggcagtgggaggacggcagcattctgagccctaacctgctgaccatcatcgagatgcagaagggcgactgcgccctgtacgccagctctttcaagggctacatcgagaactgcagcacccctaacacctacatctgtatgcagcggaccgtgtga

SEQ ID NO: 186 - protein, 492 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDKGSGATNFSLLKQAGDVEENPGPMGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 187 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 188 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 189 - DNA, 762 bp, synthetic construct
  source 1..762: mol_type = other DNA
atgtggcaactgttgctgcctacagctctgctgctgctggtgtctgccggcatgagaacagaggatctgcctaaggccgtggtgttcctggaacctcagtggtacagagtgctggaaaaggacagcgtgaccctgaagtgccagggcgcctattctcccgaggacaatagcacccagtggttccacaacgagagcctgatcagcagccaggccagcagctactttatcgatgccgccaccgtggacgacagcggcgagtacagatgccagaccaatctgagcaccctgagcgaccctgtgcagctggaagtgcacattggatggctgctgcttcaggcccctagatgggtgttcaaagaagaggaccccatccacctgagatgccactcttggaagaacacagccctgcacaaagtgacctacctgcagaacggcaagggcagaaagtacttccaccacaacagcgacttctacatccccaaggccacactgaaggactccggctcctacttctgtagaggcctcgtgggcagcaagaacgtgtccagcgagacagtgaacatcaccatcacacagggcctcgccgtgtctaccatcagcagctttttcccacctggctatcaggtgtccttctgcctggtcatggtgctgctgttcgccgtggataccggcctgtacttcagcgtcaagaccaacatccggtccagcaccagagactggaaggaccacaagttcaagtggcggaaggaccctcaggacaaa

SEQ ID NO: 190 - protein, 216 aa, synthetic construct
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 191 - DNA, 651 bp, synthetic construct
  source 1..651: mol_type = other DNA
atgggctggatccggggcagaagaagcagacacagctgggagatgagcgagttccacaattacaacctggacctgaagaagtccgacttcagcacccggtggcagaaacagagatgccccgtggtcaagagcaagtgcagagagaacgctagccccttcttcttctgttgctttatcgccgtggccatgggcatccgcttcatcatcatggtcacaatttggagcgccgtgttcctgaactccctgttcaatcaagaggtgcagatccctctgaccgagagctactgtggcccctgtcctaagaactggatctgctacaagaacaactgctaccagttcttcgacgagagcaagaattggtacgagagccaggcctcctgcatgagccagaatgcttccctgctgaaggtgtacagcaaagaggaccaggatctgctgaagctggtcaagtcctaccactggatgggcctcgtgcacatccctaccaatggctcttggcagtgggaggacggcagcattctgagccctaacctgctgaccatcatcgagatgcagaagggcgactgcgccctgtacgccagctctttcaagggctacatcgagaactgcagcacccctaacacctacatctgtatgcagcggaccgtgtga

SEQ ID NO: 192 - protein, 22 aa, synthetic construct
GSGATNFSLLKQAGDVEENPGP

SEQ ID NO: 193 - DNA, 1205 bp, synthetic construct; identical to SEQ ID NO: 90
  source 1..1205: mol_type = other DNA

SEQ ID NO: 194 - DNA, 1200 bp, synthetic construct; identical to SEQ ID NO: 91
  source 1..1200: mol_type = other DNA

SEQ ID NO: 195 - DNA, 1042 bp, synthetic construct; identical to SEQ ID NO: 87
  source 1..1042: mol_type = other DNA

SEQ ID NO: 196 - DNA, 1023 bp, synthetic construct; identical to SEQ ID NO: 88
  source 1..1023: mol_type = other DNA

SEQ ID NO: 197 - DNA, 1000 bp, synthetic construct; identical to SEQ ID NO: 84
  source 1..1000: mol_type = other DNA

SEQ ID NO: 198 - DNA, 1000 bp, synthetic construct; identical to SEQ ID NO: 85
  source 1..1000: mol_type = other DNA

SEQ ID NO: 199 - DNA, 1050 bp, synthetic construct
  source 1..1050: mol_type = other DNA
aaacttctaccaccgtcacctatctctcagggtttcctaaacatactctgaacaagtttcctcactctgccactgtgacccagaaagctatctgttctccttctcccagactctgcctcatctttcaaggcctggttccaggatcccttcctccaggaagccttccctgattgccctattccaccatacaccttttttctcggacttcatgtcatggtgcctatattccaagggctctgagccatgtacccctttatatagttaccactatttactgagtgcctactgtataccagctactgtgttggatgctctagatgtagaacctctaattatcaccatcgattcctgggtagtaggcattatttattcatcttacacagatgagaaaatggaggcccacagtggttaaataagtagcccaagattgcacagctagtagggctagtggaaagtagaggtggaatttgaactcaaatcctgcagtaactctaccattctgccttgctcttctttgtagcagtagataagttttcatggatacatacctcatccttttgattagattaagggcccctggagtgtcagtgttcattcatttgtttgatcattcattcattcaacaaacatttcttgagtccccactgtgtgccaggcccagaggttccccagcccaaggcctggcacacagtgggccttcagttagaccttgttgattgactgcgcttttccttgtctgggcagcggaactggaccagtatgtcttccaggactcccagctggagggcctgagcaaggacattttcagtaagtttgtggtgggtggggaggtcttggctcagcctgcatttcctgccttgttccctggggggtgccctaatacctgacgaccattcattgatgggcagtcagacccctctccccaaggtgggtacaatagagactcaccttgggctttcattgattgtgtgagttggtctctggtttttctcaaagtagagcacataggaccagatgaagtgatcggtgagagtatggagatgccagcag

SEQ ID NO: 200 - DNA, 1052 bp, synthetic construct
  source 1..1052: mol_type = other DNA
aagttgggcagaaaagtcagaaaagacgtgagtgagcccctccctgatccaacctagccttgcttgagacctggcctttccttgactccaaagcctgctgtgggtccaacttgcttccctcgctaagtcctgtctggttgggaggccctttaaaagccaacaggagccttaaaatgtacatctgattatttcatggccctgataaccctccaatggctacaaaatacatgccacaaggcctgtatggcccttcctccctctccaaacccactatgagacaccacacttcagccaccaccagcttctccactcctataacccatgtgccctttcaagcctcggggcctttgcagttgctatagtctctatctggaatgcccttcccccagttcttcccatggctgactcctttgaatctttctggtgttggctaaactgtcacctcttcctggaacccttctctgaccatccttccatgtagattagctcagttattctcaccttgtgtgtctttttccttgcagtttagcactcattaccatctggacatattttacgccttgctctcccactgtgaggacagggaccttgtctttcttgctcgtgactgtttccccagcatctagtgcagtgcctggtatgcagtagcacctcagtagatatctgttgaatgaaaacatctgtaaaatgggtgtaacagttaactgagtacttattatgggtctgaccatgtgtaagtcctgtatctatttattcagttcttaaacaggtgaatcgcacacagggtatgagattttaaaagtgcaaagaatattcagtgaaggctgggcgcagcggctcacacctgtaatcccagcagtttgggaggccaagggggacggatcacttgaggtcaggagtttgatacctgcctggccaacatggtgaaaccgcgtctctaccaaaaaatacaaaaattagccgggtgtggtggtgcacgcctgtaatcccagctactcgggaggctgaggcaggagaatcgcttgaacccaggaggtggaggttgcagtgag

SEQ ID NO: 201 - DNA, 973 bp, synthetic construct
  source 1..973: mol_type = other DNA
aaaaataaaaaataaaaatattaacttaatttaactttaaacaaaaaagcaggtggtctccaagaatgcaggagataactgaccgggtgcagtgtctcatgcctttaatcccagcactttggaaggccaaggcgggtggatcacccaaggtcaggagttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggt

SEQ ID NO: 202 - DNA, 1000 bp, synthetic construct
  source 1..1000: mol_type = other DNA
cgcgggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagctgcagctgaatcacacaggtaacacgggggacgtggagggacggggagaagaagaggcacagagagagaaggaaggagaggtagaaagacaagtggggagagacagagagaaagagacacagacagagacggagggagagagggagggagagatagggagggaaacggagagggggagacagagagaagacagagagg

SEQ ID NO: 203 - DNA, 1046 bp, synthetic construct
  source 1..1046: mol_type = other DNA
gtccagactcaggaggacttagttctcttcataaaaagttgatcacctttgctttctcctttgcatgggcaataaagaagtgaaaaataaattcccagtgagcttgcattctgcttgggaacaacatagcacatccattttctagagagctgtccagtccccatttgagggctgctagccacatgtcaagcaggaacccagagtataaccaggaaaatgtctgtggtagaccccagtgattcatgcctcccatctccacaccctcctgtagtcccctcccacgctgattctggactaggccacatggcttggccagtggaacacatgcaggcttgcacgtctggaactctgccacctaatttaggatcccagactctcctactggagacacaggtccttagtgacagtctgcaccaccattcagacaagtcagtagggccatcttagatcatccagccctagtcaagccaccagataactgtacccacataagtgacccctggcgagaccagcaggagaatcatgccaatgggccaatatacattctgacccacagtttcataataaaataaaatggttgtggttgtaagccactatgtttcagagtggtttgttacacagcaataaataactaatatagtaggcataccatcaagtccaaagtaggtagagaagaatgtaaatagcagagcaaaacagcatgactggtggctgggaggcttaaaactgggacaggatcagagtcatgaaagaagtcaaagaaatggttcagaagtaaggctgagactgacttacaaaagctgaaagtccctttaagttggtgtttggtgcattggcaggggcaggtatggtgacttaaaagagccatgctcaacaagatcaagcacaacacaatcacgggtcaccccagcagaccttagcgagtctagccatttctttggtggtggtcacagtcatgcttcagcccagtttccacttggacaaatggtacatattttcaatgagatgaaaattaagatacaatccatgtgctcagagagtgatca

SEQ ID NO: 204 - DNA, 1053 bp, synthetic construct
  source 1..1053: mol_type = other DNA
atcatgtctgctcgacagctctgactaaacactgtgccccaaagtgttgaggaattgggaaaacctagctgagttagtggtctcttttctgttacaataaagctcataatgaaaattagccttctttgttcttccccaagtctttctttctagacgaaactactttcaactgttttaacttccttactgttaacttccatatttctagatagtatggtcagactgttatttcttgacttttaaatgtaagatattatctactgacttccttctatgtaagatgaggattgagctctcttacccttctcccatttcctcatccttccaacataaatatattttgggattatatcaacattcaatgttacttaaagtgaccttgtaaatattttcacaactgagccatgtttgatttgtatacttatgtttactttactgtttttcctgaagttaataattgccttgaatttatttatttctttaaaaatgtttcattactcaggactgtagtttacattacgattctttgtgttatacagttgatgggtttcttttctttcttaatttctttaaaaaatagagatggggtcttactatattacccaggctggtcttgaagtcctgggctcaagtgatcttcctgtctcagcctaccaagtagctgagactataggtgcaaaaaagccactatacctggctagtttacaggttttaacaaatgcattatgccacgtatccattattacaggatcacacaagatattttcattaccctgaaagcatccctgtgttccaccaattcatcctgcctccatgagccgctggcaaccactgatctctatagttttgccttttctaaaatgtcatataattggaatcatacagtctgtagcattttcagactagcttttaaaatttggcaatatgcatttaaggttcctccttaaatgtgaagggcatagccaatgtggcttgatagctcatttctttttattggtgaatatttcattgtctggatgttccacagtttgtttatcctcgagagcttggcgt

SEQ ID NO: 205 - DNA, 1015 bp, synthetic construct
  source 1..1015: mol_type = other DNA
ttatgtagtgttcctaatactatttaggttatgcataagttaactgtgtcacatagcattttctgtcatatacaaatatttaattttttataatttttaataaataatttttaataatttcttagaacatttcagccattaagaaaagaccatttaattacctctattgattttcattaatgtgtataaattactaattaaaagtaatgtagtgcaagaaatttttatatatatatatgtcaactttactattactcatgtgcagaccacatagtcttaaccattactggatttcaaaattaacataaaaatgagtgcagaagtttttctaatacaacattttaatttttaacacaggtaaaacatcagactttaagaaatatatttttattcctgaacttctttattccttagtaatttattgcttctcattgccccagcaataatattttgtcaaatgcagaaaatttatcttttttttttgagtcggagtctcactctgtcgcccaggctggagtgcagtggcacaatctccgctcactgcaacctctgccgcccatgttcaagagattctcctgcctcagcctcccgagtagctaggactacaggcgcctgacatcatgcccgactactttttgtatttttagtagagacggagtttcaccgtgttagccaggatggtctcgatctcctgacctcgtgatcggcatgcctcggcctcccaaagtgctgggattacaggcgtgagccaccgcgcccggcctaaaaatctttttttaaaacaaatattcataagaaacgtgtttaggcttgaagaaaatcagagaaagaactttagattatttaatgcaaaatgagctccaatactcgttctccacctcacccttttaattgcactagggaatcctgtatataaaccatttattaacttcttaactactgttattatagagtacagtccctgacatcacacactgcagagatggataaccaaggagtaatctactcagacctg

SEQ ID NO: 206 - DNA, 1020 bp, synthetic construct
  source 1..1020: mol_type = other DNA
aatctgcccccaaacccaaagaggcagcaacgaaaacctaaaggcaataaaaactccattttagcaactgaacaggaaataacctatgcggaattaaaccttcaaaaagcttctcaggattttcaagggaatgacaaaacctatcactgcaaaggtaaagcatttaaaagatcctcaatataacagtctaggatgtgcagcttggggtacaggaatgtggggaaagagaagggagtgctcatatatcttctatttgcaaagatcagaattccaagttgagatatgctatttcaatgtaaagtatgaagactgattgaactcattgttgaagtttgtagtctttgtcaaataattcatggagcattatttttcctgaaaattcaatggtatattattctgagaaaaagattacaatgggagatgagggtttggggtccaagtttctctgtatgattcctgtgcattcaggttctcttgtctgtgaatcttctaaacgactgtatccacctctcctttcgcactgttcccatttctctccctgcagatttaccatcagctccagagaagctcattgttgggatcctgggaattatctgtcttatcttaatggcctctgtggtaacgatagttgttattccctgtaagtctattttcgaagattacaaggggaattttcacgttaatgattgaatgtgcctctaaacatttcatattttcagggaatagagttctcattgtaatgtatatatttggactaaatgtggaatgattattctgaatttgtcaaagaataaatgaaagaataattgttgaaagtattcgcttctgatgcaatcgtatgtatatatttggatttcataactcaaaaatatgttctaggagtctgaaaaaccttactgagaaatagaaattaatttttgaaagtagttaaatcaagaattataagaactatatgagatggtgaaatttggttctttagatctatgaaatacttttccaaaaaaccaccattactttatc

SEQ ID NO: 207 - DNA, 1000 bp, synthetic construct
  source 1..1000: mol_type = other DNA
ctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagc

SEQ ID NO: 208 - DNA, 1000 bp, synthetic construct
  source 1..1000: mol_type = other DNA
tgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagg

SEQ ID NO: 209 - DNA, 1050 bp, synthetic construct
  source 1..1050: mol_type = other DNA
cttgctgctctggaacaggccaagagcttttctgcctcagagtctttgcacctgccatttcctctgcttgggaaatgtttgccccaagggagttgggtgacttgatcgctcacattacttaggtctctgcttgaatgtcacagatgttctcttaataaagaagaggcaagaaaagccactttattatttattaaactcccgcatagagtgcagtattattactgtgtgccagaccctgcttcaaacacattccatggactataaaattgcatctctgagcagctcctagagctggtagtaacaacttacatttactgggtgattaccatgtgccaggtattgtgctaaacacgttgtagatattaactcacttaatcctcgtaacaatcccatgaagtaggtactgctactatcccggctttacatctgaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccctatggccaactgcgagttcagcccgg

SEQ ID NO: 210 - DNA, 1050 bp, synthetic construct
  source 1..1050: mol_type = other DNA
tgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatagaactcgcagatgcagggtgtcgctgacattttcaactttttctgcggtttccgcccgctgtctctgacccgaaagtgcccccggacggttacagaggacacttaagtggtttgcaaagcctgtggtaggggaggagggtgtagaagggccaaaccacggaacttagttttattcatttatataaagcagcactccgattctttttgcgcggcctgaaatgcatgtgaccagagaagtaattaacaaaacaatgtcaacttctaaaaccgagacattacttagatgataaggcgcagcaactcggtgaatctgtacaaaccttggaaaaaaaacacattagtctatgggaccttccagttttctcatgctcctttccagctactaacctctcctaaagggaacaaccactttttggatttgattcccaggcctcgctttcaccgggaaattatcgttgcttgtaaaacaga

SEQ ID NO: 211 - DNA, 1052 bp, synthetic construct
  source 1..1052: mol_type = other DNA
gaaggactttagacatggggttcgcatgtctcagatggccctgaaggtactgatccaggctctggtgctcctggaaggcaagactcagattctgctccatctcctcccatctctgggcgggtctctggcatctctggcccatgagggtcaatctgtgtggaggggacaagctctgagcatgtgtgggtctgaggttcctcttccatgcagggctgaggtctcctgctcctccccagcttcctgtccggccctgtagtccttcccctccactcccttcctcttttctgctcacacaggaagccctggaagctgcttcctcagacatgccgctgctgctactgctgcccctgctgtgggcaggtgagtggctgtggggagaggggttgtcgggctgggccgagctgaccctcgtttccccacaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacaggtgaggcacaggcttcagaagtggccgcaagggaagttcatgggtactgcagggcagggctgggatgggaccctggtactgggaggggtttaggggtaaagcctgtcgtgcttagcgggggagcttgaccagaggttgatcttctctcaggccctcacctggaccctccctcctgattctgcatcccctctttctcctcactagacttgacccacaggcccaaaatcctcatccctggcactctagaac

SEQ ID NO: 212 - DNA, 1052 bp, synthetic construct
  source 1..1052: mol_type = other DNA
ccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctgtaagtgctgggccaggatgctggggtccctgagggtgtaggggagacaggatgggctggtgctggggacatttagtgtcctggaggcctggctgagttcgggagccagaaggacatgagccctgtcccttctgcatttctgtggtttctggcaggagtaaggggaaatgcctacccttatctcatctctacccccaactgaaggaaatcctctcttcctctcctagatgttccacagaacccaacaactggtatctttccaggagatggctcaggtaggaaggagcctccccgcctggggctgttactgacattgagtctgtgtcaggtttggtcagatctggactttcagagtcaaatgttcagaggcaaggcctgcagttagacacgggtagacatcaggcaccttggaaaaggatatttggggatgactagcaacttcccccttgcccatccaaataatgctctttgtctccctcctgtctctgaatgtcttggggtattttatttttaattgatatgtaataatagtacatatttatggatggcatagtgatgtttccatactaataatgtatagtaatcagatcagggtaatagcatatccatcatcttgaacatttattatttcattgttgttgggaacattcaatatcccctttctagctatttgaagctatctattattgttaagcatagtcatcctacagtggtatagaacaccagaacttattcttcctttccaggtgtaatctagtatcctttaacaaatctctctccttatcattgttcccctaaccttcccagcccttattatt

SEQ ID NO: 213 - DNA, 800 bp, synthetic construct
  source 1..800: mol_type = other DNA
tgcttcataagtgaaggagaaataaaatatagacaagtgaacgctgaaagattttgtcaccaccaggcctgccctacaagagctcctgaaggaagcgctaaacatggaaaggaacaaccggtaccagccactgcaaaaacatgccaaattgtaaacaccattgaggccaggaagaaactgcatcaactaacgagcaaaataaccagctaacatcatcatgacaggatcaaattcacacataacaatattaaccttaaatgtaaataggctaaatgctccaattaaaagacacagactggcaaactggataaagagtcaagacccatcagtgtgctgtattcaggaaacccatctcacgtgcagagacacacataggctcaaaataaagggatggaggaagatctaccaagcaaatggaaaacaaaaaaaggcaggggttgcaatcctagtctctgataaaacagactttaaaccaacaaagatcaaaagagacacagaaggccattacataatggtaaagggatcaattcaacaagaagagttaactatcctaaatatatatgcacccaatacaggagcacctagattcataaagcaagtccttagagacctacaaagagacttagactcccacacaataataatgggagactttaacaccccactgtcaacattagacagatcaatgagacagaaaattaacaaggatatccaggaattgaactcaactctgcaccaagcggacctaatagacatctacagaactctccaccccaaatcaacagaatatacattctttt

SEQ ID NO: 214 - DNA, 800 bp, synthetic construct
  source 1..800: mol_type = other DNA
tccaaaattgaacacatagttggaagtaaagcactcctcagcaaatgtaaaagaacagaaagtacaacaaactgtctctcagaccacagtgcaatcaaactaaaactcaggattaagaaactcactcaaaaccgctcaactacatggaaactgaacaacctgctcctgaatgactactgggtacataacgaaatgaaggcagaagtaaagatgttctttgaaaccaacgagaacaaagacacaacataccagaatctctgggacacattcaaagcagtgtgtagaggaaaatttatagcactaaatgcccacaagagaaagcaggaaagatctaaaattgacagcctaacatcacaattaaaagaactagagaaacaagagcaaacacattcaaaagctagcagaaggcaagaaataactacaatcagagcagaactgaaggagatagagacatacaaaaaacccttcaaaaaatcaatgaatccaggagctggttttttgaaaagatcaacaaaattgatagaccactagcaaaactaatacagaagagagaagaatcaaatagacacaataaaaaatgataaacgggatatcaccactgatcccacagaaatacaaactaccatcaaagaatactataaacacctctatgcaaataaactagaaaatctagaagaaatggataaattcctcgacacatacaccctcccaagactaaaccaggaagaagctgaatctctgaatagaccaataacaggctctgaaattgaggcaacaattaacaccttaccaaccaataa

SEQ ID NO: 215 - DNA, 776 bp, synthetic construct
  source 1..776: mol_type = other DNA
ggtgcctgccaccacgccaggttaattttttgtatttttagtagagacagggtttcaccgtgttagccaggatggtctcgatctcctgacctcatgatccacccatcttggcctcccaaagtgctgggattacaggcatgaaccactgcgcccggccgcatcgctagtttttaaaaactttttgtagagacagattcttactatgttgccaaggctggtctcaaactcctggcctcaagagatcctccagtcttcggcctcccaaaaagatgggattacaggcatgagccacctcacctggcctcttttttttgtatattacctgatctcaggtattctgctatagcaacagaaagacgaagacagaatccttagctgtctgcaagtgtgcatgccattttcatcatctgaagagtcagcgagtgtcttaggtggagtcttgcaaaagcaggccctgagccaaagatttggatgcaaatgacttgttaagaaagggctcttcgagaccgtgccattgcactccagcccgggcaagaagagtgaaactctgtttcaaaaaaaaaaaagtggggggctcctaggaaaagaacagtaaaggagtgggggatgaaggacagggaatgggaagaagccaagcgagagcatgatttccgaagtcctacactcagcctgatcacacgggaagctttagaacaaagaacacacctcagagtttttcctgcctcaacacaaaggagctgggctttggtgctcttcatcagcctgtctttggctat

SEQ ID NO: 216 - DNA, 780 bp, synthetic construct
  source 1..780: mol_type = other DNA
gaaacataaaactccgaggtacttccggctccctccagtgtctgagggtaatctgcaggactgagggtaattgcagatgctagctgttagcagcaaactatgcaaaagctgaggactggcttataaagccagatctgggtgagtcatgtttcctgtcaacatcctctgctgggcccataacacatgcaaccccaaacttccattacaagttcaaagtttctaaggggatagcattacagtgtgtatgatattggactcagacctgagtttgaatcctaattccacaaaagaaattggaaaagagtcatattgctgacttgaccctttgtcaccatatccataaaatgggataattattcctatatcataaatttacttatttattcacttagtcatttgttaaataaatatggagtgtctactttgtgccgggcactctttttagggtggttctgagaaggggatggcaatgagaagggctctctaagatgcaagactccaggcaactgcttttacttccagtggttctttattttcacagctcattagagcaaattaccacagcagggagatacaggttgagtatcccttatccgaaaagcctggcaccagaagtgttttaaattttggatttttttttgatttttggaatatttgttaattatcagttgagcatctctaatgtgaaaatctaaaatccaaaatgccccagtgaccctttcctttgagcatcatgttggtgctcaaaaagtttgagatgttggagcatttaggatttca

SEQ ID NO: 217 - DNA, 20 bp, synthetic construct
  source 1..20: mol_type = other DNA
tctgtcccctccaccccaca

SEQ ID NO: 218 - DNA, 20 bp, synthetic construct
  source 1..20: mol_type = other DNA
cattctctgctggatgacgt

SEQ ID NO: 219 - DNA, 20 bp, synthetic construct
  source 1..20: mol_type = other DNA
ccttggggctctgacaggta

SEQ ID NO: 220 - DNA, 20 bp, synthetic construct
  source 1..20: mol_type = other DNA
tgcccaacttctgctggcat

SEQ ID NO: 221 - DNA, 20 bp, synthetic construct
  source 1..20: mol_type = other DNA
gtcccattggtcgcgggctt

SEQ ID NO: 222 - DNA, 20 bp, synthetic construct
  source 1..20: mol_type = other DNA
gtcagagctgtgatcactct

SEQ ID NO: 223 - DNA, 21 bp, synthetic construct
  source 1..21: mol_type = other DNA
actcagacctgaatctgcccc

SEQ ID NO: 224 - DNA, 20 bp, synthetic construct
  source 1..20: mol_type = other DNA
gagtctctcagctggtacac

SEQ ID NO: 225 - DNA, 19 bp, synthetic construct
  source 1..19: mol_type = other DNA
ccgagaccgtcctggcgcg

SEQ ID NO: 226 - DNA, 21 bp, synthetic construct
  source 1..21: mol_type = other DNA
tctgcagggaaacaagagacc

SEQ ID NO: 227 - DNA, 20 bp, synthetic construct
  source 1..20: mol_type = other DNA
cggcaccacaccacaccgat

SEQ ID NO: 228 - DNA, 20 bp, synthetic construct
  source 1..20: mol_type = other DNA
acctcgttccacaaccctgg

SEQ ID NO: 229 - protein, 376 aa, synthetic construct
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 230 - protein, 376 aa, synthetic construct
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 231 - protein, 741 aa, synthetic construct
MWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 232 - protein, 1135 aa, synthetic construct
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSTSGSGKPGSGEGSTKGMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 233 - protein, 1139 aa, synthetic construct
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANSGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 234 - protein, 1138 aa, synthetic construct
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 235 - protein, 1138 aa, synthetic construct
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 236 - DNA, 3408 bp, synthetic construct
  source 1..3408: mol_type = other DNA
atggcctcatatccctgccaccagcatgcgagtgcgtttgatcaagctgcacggtcaagaggacattccaataggcgaactgcactgagacctaggcgacaacaagaagcaacggaggtccgccttgagcagaagatgcctactcttcttagggtttacatcgatggtccgcacggaatgggtaagaccacgactactcaactgcttgtcgcattgggctccagggatgacatagtttacgttccggaacctatgacttattggcaggtgttgggggcatccgaaaccatagctaatatttataccacgcaacaccgcttggaccaaggagagatctctgcgggagatgcagcggtggtaatgaccagcgcacaaatcaccatggggatgccgtatgccgtaacagacgcggtgctggccccccatatcggtggtgaagccggttcaagtcatgcgcctccgccggcgctgacattgatctttgatcggcatcctattgcggcactcctgtgttatccagccgcacgctatctgatgggaagcatgaccccacaagcggttctggcctttgtggcattgatcccaccaacattgcctggaactaatattgtactgggcgctctccccgaggatcgacatattgataggctcgcgaaacgccaacgccctggtgaaaggcttgatttggccatgctcgcggctatacgccgcgtctatgggttgctcgccaatactgtgagatacctgcagggcggggggtcatggcgcgaggattggggtcagttgtcaggcacggcagtgcccccacagggggcggagcctcagtcaaatgcaggcccaagaccgcatattggtgatactctcttcacgttgtttcgggcgccagaattgcttgcccccaacggtgatctttataacgtcttcgcatgggccctcgatgtcttggccaagaggctgcgaccgatgcacgtttttattctcgactatgaccagtcacctgcgggatgcagggatgcgttgctccagttgacaagtggcatggtgcagacacatgtgacgacacccggatcaatacctacgatatgtgatctcgcaaggacctttgcgagggaaatgggggaagctaatggctcaacttctggatccggaaagcccggtagcggcgaaggttctacaaagggtatgtggaacctgttggcaagacgcccccgctggctctgtgccggtgctctggtactggcggggggattttttctcttgggatttctttttgggtggttcataaaaagttctaatgaggccacaaatatcactccgaaacacaatatgaaggcatttctggacgagctcaaagcggaaaatattaagaaattcctgtataactttactcagatacctcatctggctggcaccgagcaaaacttccagttggctaagcagattcaatcacagtggaaagagttcggtctcgatagcgttgaattggcacactacgatgtccttcttagctatcctaataaaacacatccgaactacataagtatcattaatgaggacggcaacgaaattttcaacacctctctttttgaacctcctccgccgggatatgaaaacgtgagcgatatcgttccccccttttccgcgttctcaccacagggaatgccggagggtgaccttgtttatgtaaattatgccagaacggaagatttctttaaacttgaacgcgatatgaaaatcaactgctctggtaagattgttattgcccgatacgggaaggtattcaggggtaacaaagtgaaaaacgctcagctggcgggtgccaaaggagtgatcctttattctgaccctgcggattatttcgcaccaggcgtaaaatcatatcctgacggttggaaccttcctggaggtggagtacagcgcggaaatatattgaatcttaacggagccggtgacccactgactcctggataccccgcaaacgagtatgcctatcgacgcggcattgccgaagcggtgggactgccctcaatacctgtacatcctattggatactatgatgctcagaaacttttggagaagatgggtggaagtgccccgcctgatagttcctggagaggctcccttaaggttccatataacgtaggtccagggtttacgggcaacttttcaacacaaaaggtaaagatgcatatacattcaactaacgaggtgacgaggatatacaatgtaatcggaactctgaggggagccgtagagcctgatcgatatgtcatcttggggggccacagggatagttgggtctttggtggaattgatcctcagtcaggtgcggctgtagtccacgagattgtccgctctttcggcacgctgaagaaggaggggtggagaccccgaaggactattttgtttgcctcttgggatgctgaagaatttggtctgctcggatcaacggagtgggctgaagagaattctaggttgttgcaagaacgcggtgtggcctacataaatgcggacagtagtatagaaggcaattacacacttcgagtggattgcaccccgcttatgtacagtctggtacataacctgacgaaggagcttaaatcacctgatgaaggattcgaggggaaatccctttacgaatcatggactaaaaagtcaccttcccctgaatttagtgggatgccgcgcataagtaaactcgggtccggaaacgacttcgaagttttcttccaacgattgggtatcgcctctggacgagcacggtacaccaaaaattgggaaacgaacaaattttccggatatcctctctaccactctgtctatgaaacctacgagctggtggaaaagttttacgatccgatgtttaagtaccatttgaccgtcgcccaggtgcggggaggaatggtctttgaattggcaaatagtatagtccttccttttgattgtcgagattatgccgtcgtccttaggaagtatgctgacaagatttattcaatatctatgaagcacccccaagaaatgaagacctactcagtgtcctttgactccctgttctccgctgtgaagaacttcactgagatcgcctctaagttctcagagcgactgcaagattttgacaagagtaaccccattgttcttaggatgatgaatgaccagctcatgtttttggagagagcatttattgatccgctgggccttcccgaccggccattttacagacacgttatctatgctccttcaagtcacaataaatatgcaggagaatcctttcctgggatctacgatgccctcttcgacatagaaagtaaggttgatccctccaaggcgtggggggaagttaaacgacagatatatgtcgctgcattcaccgtacaggccgccgcagagacacttagtgaggttgcgtga

SEQ ID NO: 237 - DNA, 3420 bp, synthetic construct
  source 1..3420: mol_type = other DNA
atggcaagttacccttgccaccagcatgctagcgctttcgatcaagcggcccgcagtcggggccatagcaatagaaggaccgcattgcgcccgcgccgacagcaggaagctaccgaagtacgactggaacaaaaaatgcccactcttctgagagtatacatcgatgggccacacggcatgggcaaaacgacgactacccaactcttggtagctcttggtagccgggatgatatcgtatatgtgccagaacctatgacctattggcaggtcctcggcgccagtgaaaccatcgccaacatatatacgacgcaacataggcttgaccagggtgaaatctccgcaggtgacgcggcagtggtcatgactagcgcccaaatcacgatgggaatgccttatgcggtgactgacgcagtactcgctcctcacattggaggtgaagcggggagctcccacgcaccgccgcccgctcttacgctcattttcgatcgccaccctatagctgccctgctctgttatcccgcggccaggtacttgatggggtccatgaccccccaggcggtgctggccttcgttgcgttgataccgccaactctccccggcactaatattgttctcggagcacttccagaagacaggcatattgacaggttggctaagcgccaaaggcccggtgaacgactcgacctggctatgcttgctgccatccgccgcgtctatgggctgctggctaatacggtcaggtatcttcaaggcggcggatcttggagggaagattggggacagctcagtggaacggctgtacctccacaaggggccgaacctcagtcaaatgcaggtcctcgccctcacattggagatacactttttactcttttccgggctccagaactgctggcaccaaatggcgacctgtacaatgtttttgcctgggctctggatgttttggcaaaaaggcttcggccgatgcacgtatttattttggactatgatcagtcaccggctggttgtagagacgcattgcttcaacttacatccgggatggtacaaacgcatgtaacaaccccagggtcaattccaactatctgcgatctcgcccgcacattcgcaagagaaatgggtgaggctaactctggcagtggtgaaggccgcggatctctcctgacttgtggggatgtcgaagaaaacccgggacccatgtggaacctgttggcaagacgcccccgctggctctgtgccggtgctctggtactggcggggggattttttctcttgggatttctttttgggtggttcataaaaagttctaatgaggccacaaatatcactccgaaacacaatatgaaggcatttctggacgagctcaaagcggaaaatattaagaaattcctgtataactttactcagatacctcatctggctggcaccgagcaaaacttccagttggctaagcagattcaatcacagtggaaagagttcggtctcgatagcgttgaattggcacactacgatgtccttcttagctatcctaataaaacacatccgaactacataagtatcattaatgaggacggcaacgaaattttcaacacctctctttttgaacctcctccgccgggatatgaaaacgtgagcgatatcgttccccccttttccgcgttctcaccacagggaatgccggagggtgaccttgtttatgtaaattatgccagaacggaagatttctttaaacttgaacgcgatatgaaaatcaactgctctggtaagattgttattgcccgatacgggaaggtattcaggggtaacaaagtgaaaaacgctcagctggcgggtgccaaaggagtgatcctttattctgaccctgcggattatttcgcaccaggcgtaaaatcatatcctgacggttggaaccttcctggaggtggagtacagcgcggaaatatattgaatcttaacggagccggtgacccactgactcctggataccccgcaaacgagtatgcctatcgacgcggcattgccgaagcggtgggactgccctcaatacctgtacatcctattggatactatgatgctcagaaacttttggagaagatgggtggaagtgccccgcctgatagttcctggagaggctcccttaaggttccatataacgtaggtccagggtttacgggcaacttttcaacacaaaaggtaaagatgcatatacattcaactaacgaggtgacgaggatatacaatgtaatcggaactctgaggggagccgtagagcctgatcgatatgtcatcttggggggccacagggatagttgggtctttggtggaattgatcctcagtcaggtgcggctgtagtccacgagattgtccgctctttcggcacgctgaagaaggaggggtggagaccccgaaggactattttgtttgcctcttgggatgctgaagaatttggtctgctcggatcaacggagtgggctgaagagaattctaggttgttgcaagaacgcggtgtggcctacataaatgcggacagtagtatagaaggcaattacacacttcgagtggattgcaccccgcttatgtacagtctggtacataacctgacgaaggagcttaaatcacctgatgaaggattcgaggggaaatccctttacgaatcatggactaaaaagtcaccttcccctgaatttagtgggatgccgcgcataagtaaactcgggtccggaaacgacttcgaagttttcttccaacgattgggtatcgcctctggacgagcacggtacaccaaaaattgggaaacgaacaaattttccggatatcctctctaccactctgtctatgaaacctacgagctggtggaaaagttttacgatccgatgtttaagtaccatttgaccgtcgcccaggtgcggggaggaatggtctttgaattggcaaatagtatagtccttccttttgattgtcgagattatgccgtcgtccttaggaagtatgctgacaagatttattcaatatctatgaagcacccccaagaaatgaagacctactcagtgtcctttgactccctgttctccgctgtgaagaacttcactgagatcgcctctaagttctcagagcgactgcaagattttgacaagagtaaccccattgttcttaggatgatgaatgaccagctcatgtttttggagagagcatttattgatccgctgggccttcccgaccggccattttacagacacgttatctatgctccttcaagtcacaataaatatgcaggagaatcctttcctgggatctacgatgccctcttcgacatagaaagtaaggttgatccctccaaggcgtggggggaagttaaacgacagatatatgtcgctgcattcaccgtacaggccgccgcagagacacttagtgaggttgcgtga

SEQ ID NO: 238 - DNA, 3382 bp, synthetic construct
  source 1..3382: mol_type = other DNA
atggccagctatccttgtcaccagcacgccagcgcctttgatcaggccgcaagatctagaggccacagcaacagaagaacagccctgcggcctcggagacagcaagaggctacagaagttcggctggaacagaagatgcccacactgctgcgggtgtacatcgatggccctcacggcatgggcaagaccaccacaacacagctgctggtggccctgggcagcagagatgatatcgtgtacgtgcccgagcctatgacctactggcaggttctgggagccagcgagacaatcgccaacatctacaccacacagcaccggctggatcagggcgaaatttctgctggcgacgccgccgtggttatgacatctgcccagatcaccatgggcatgccttacgccgtgacagatgctgtgctggcccctcacattggcggagaagccggatcttctcatgcccctccaccagctctgaccctgatcttcgacagacaccctatcgcccatctgctgtgttatcctgccgccagatacctgatgggcagcatgacacctcaggccgtgctggctttcgtggccctgattcctcctacactgcccggcaccaatatcgtgctgggagccctgcctgaggaccggcacattgatagactggccaagagacagcggcctggcgagagactggatctggctatgctggccgccatcagaagagtgtacggcctgctggccaacaccgtgcggtatcttcaaggcggcggatcttggagagaggactggggacaactgagcggcacagcagttcctccacaaggcgctgagcctcagtctaacgctggacccagacctcacatcggcgacaccctgtttaccctgttcagagcccctgagctgctggctcctaacggcgacctgtacaacgtgttcgcctgggctcttgacgtgctggcaaaaagactgcggcccatgcacgtgttcatcctggactacgatcagtcccctgccggctgtagagatgctctgctgcagctgacaagcggcatggtgcagacccacgttacaacccctggcagcatccccaccatctgtgacctggccagaaccttcgccagagagatgggagaagccaacggcagcggcgaaggcagaggatctctgctgacatgtggcgacgtggaagagaaccccggacctatgtggaacctgctggctagacggcccagatggctttgtgctggtgctctggttctggctggcggctttttcctgctgggcttcctgtttggctggttcatcaagagcagcaacgaggccaccaacatcacccctaagcacaacatgaaggcctttctggacgagctgaaggccgagaatatcaagaagttcctctacaacttcacgcagatccctcacctggccggcaccgagcagaattttcagctggccaagcagatccagagccagtggaaagagttcggcctggactctgtggaactggcccactacgatgtgctgctgagctaccccaacaagacacaccccaactacatcagcatcatcaacgaggacggcaacgagatcttcaacaccagcctgttcgagcctccacctcctggctacgagaacgtgtccgatatcgtgcctccattcagcgctttcagccctcaagggatgcctgagggcgatctggtgtacgtgaactacgccagaaccgaggacttcttcaagctggaacgggacatgaagatcaactgctccggcaagatcgtgatcgcccgctacggcaaagtgttccggggcaacaaagtgaagaacgcccagctggcaggcgccaaaggcgtgatcctgtatagcgaccccgccgactattttgcccctggcgtgaagtcttaccccgacggctggaatcttcctggtggcggagtgcagagaggcaacatcctgaaccttaacggcgcaggcgaccctctgacacctggctatcctgccaatgagtacgcctacagacggggaattgccgaggctgtgggactgccttctatccctgtgcaccccatcggctactacgacgcccagaaactgctggaaaagatgggcggaagcgcccctcctgactctagttggagaggctctctgaaggtgccctacaatgtcggcccaggcttcaccggcaacttcagcacccagaaagtgaaaatgcacatccacagcaccaacgaagtgacccggatctacaacgtgatcggcacactgagaggcgccgtggaacccgacagatatgtgatcctcggcggccacagagatagctgggtgttcggaggaatcgaccctcagtctggtgccgctgtggtgcacgaaatcgtgcggtcttttggcaccctgaagaaagaaggctggcgccccagacggaccatcctgtttgcctcttgggacgccgaggaattcggactgctgggatctacagagtgggccgaagagaacagcagactgctgcaagaaagaggcgtggcctacatcaacgccgacagcagcatcgagggcaactacaccctgagagtggactgcacccctctgatgtacagcctggtgcacaacctgaccaaagagctgaagtcccctgacgagggctttgagggcaagagcctgtacgagagctggaccaagaagtccccatctcctgagttcagcggcatgccccggatctctaagctcggctctggcaacgacttcgaggtgttcttccagcggctgggaatcgcttctggcagagccagatacaccaagaactgggagacaaacaagttctccggctatcccctgtaccacagcgtgtacgagacatacgagctggtcgagaagttctacgaccccatgttcaagtaccacctgacagtggcccaagtgcgcggaggcatggtgttcgaactggccaatagcatcgtgctgcccttcgactgcagagactatgccgtggtgctgcggaagtacgccgataagatctacagcatcagcatgaagcacccgcaagagatgaagacctacagcgtgtccttcgatagcctgttcagcgccgtgaagaacttcaccgagatcgccagcaagttcagcgagcggctgcaggacttcgacaagagcaacccaatcgtgctgaggatgatgaacgaccagctgatgttcctggaacgcgccttcatcgacccactgggcttgcccgatagacccttctaccggcacgtgatctatgcccctagcagccacaacaaatacgccggcgagagcttccccggcatctacgatgccctgttcgacatcgagagcaaggtggacccatctaaggcctggggcgaagtgaagcggcagatctacgtggccgcattcacagtgc

SEQ ID NO: 239 - DNA, 1272 bp, synthetic construct
  source 1..1272: mol_type = other DNA
atgcgcatctctaagcctcatctgcgatccatcagtatccaatgttacctgtgtctgctgcttaatagtcattttctgactgaagccggaatccatgtctttatcctgggctgcttcagtgcgggattgcccaaaactgaagccaactgggttaacgtgatctctgatttgaagaaaatagaagaccttatacaaagtatgcatattgatgccacgctctataccgaatcagacgttcatccctcatgcaaagtcacagctatgaagtgttttctgctggagctgcaggttattagtctggagagtggagacgccagcatccacgatacggtcgagaatctcattatcttggcaaataattcactctcttcaaacggaaacgtgaccgaatcagggtgcaaggagtgcgaggaactcgaagaaaagaatatcaaagagttcctgcagagcttcgtgcacatcgtgcaaatgttcatcaatacctctgggggaggtggttcagggggtggaggttctggtggggggggctcaggaggagggggtagcggcggtggtggaagcatcacgtgtcccccaccgatgtctgtagaacacgctgacatctgggtcaaatcttactctctgtactctcgcgaacgctacatttgcaactcagggtttaaacggaaagccggaacctctagtctgacggagtgtgtgctgaataaggcgacaaacgtagcgcactggaccacccctagtctcaagtgcataagggacccagctttggtacaccagaggcctgcaccgccctccaccgtcactacggctggcgtaacaccgcaaccggagtctctgagtcctagcggaaaagagcccgccgcaagtagcccttcaagtaacaacaccgccgcgacgacggcggctattgtgcccggtagtcaattgatgccttccaagtctccgtccacaggcacgacagagataagttcacacgaaagtagccatgggactccttcacagacgaccgcaaagaattgggagttgacggcttcagcctcacaccagcccccgggcgtgtatccgcagggacatagcgataccacggtagccataagtactagtacagtattgctgtgcggactctctgccgtcagtcttcttgcttgttatttgaaatccagacagacgccaccgctggcctccgtggaaatggaagcgatggaagccctccctgtaacgtggggaacaagcagtcgagacgaagatctggagaactgctcacatcacttg

SEQ ID NO: 240 - DNA, 20 bp, synthetic construct
  source 1..20: mol_type = other DNA
ctcacgtcatccagcagaga

SEQ ID NO: 241 - DNA, 21 bp, synthetic construct
  source 1..21: mol_type = other DNA
gagtggccgggttctagagtg

SEQ ID NO: 242 - protein, 163 aa, synthetic construct
TTTPAPRPPTPAPTIASQPLSLRPEACRPAAGGAVHTRGLDFACDIYIWAPLAGTCGVLLLSLVITCSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 243 - protein, 163 aa, synthetic construct
TTTPAPRPPTPAPTIASQPLSLRPEAARPAAGGAVHTRGLDFAADVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 244 - protein, 163 aa, synthetic construct
IEVMYPPPYLDNEKSNGTIIHVKGKHLCPSPLFPGPSKPFWVLVVVGGVLACYSLLVTVAFIIFWVCSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 245 - protein, 169 aa, synthetic construct
TTTPAPRPPTPAPTIASQPLSLRPEACRPAAGGAVHTRGLDFACDFWVLVVVGGVLACYSLLVTVAFIIFWVCSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 246 - protein, 234 aa, synthetic construct
TTTPAPRPPTPAPTIASQPLSLRPEACRPAAGGAVHTRGLDFACDIYIWAPLAGTCGVLLLSLVITLRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 247 - protein, 245 aa, synthetic construct
TTTPAPRPPTPAPTIASQPLSLRPEACRPAAGGAVHTRGLDFACDIYIWAPLAGTCGVLLLSLVITCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLLTRKKKALRIHSVEGDLRRKSAGQEEWSPSAPSPPGSCVQAEAAPAGLCGEQRGEDCAELHDYFNVLSYRSLGNCSFFTETG

SEQ ID NO: 248 - protein, 204 aa, synthetic construct
TTTPAPRPPTPAPTIASQPLSLRPEACRPAAGGAVHTRGLDFACDIYIWAPLAGTCGVLLLSLVITCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLAVSLSKMLKKRSPLTTGVYVKMPPTEPECEKQFQPYFIPIN

SEQ ID NO: 249 - protein, 208 aa, synthetic construct
TTTPAPRPPTPAPTIASQPLSLRPEACRPAAGGAVHTRGLDFACDIYIWAPLAGTCGVLLLSLVITCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLAVSLSKMLKKRSPLTTGVYVKMPPTEPECEKQFQPYFIPINYSSV

SEQ ID NO: 250 - protein, 167 aa, synthetic construct
TTTPAPRPPTPAPTIASQPLSLRPEACRPAAGGAVHTRGLDFACDIYIWAPLAGTCGVLLLSLVITCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLYSSV

SEQ ID NO: 251 - protein, 150 aa, synthetic construct
TTTPAPRPPTPAPTIASQPLSLRPEACRPAAGGAVHTRGLDFACDIYIWAPLAGTCGVLLLSLVITHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 252 - protein, 211 aa, synthetic construct
TTTPAPRPPTPAPTIASQPLSLRPEACRPAAGGAVHTRGLDFACDIYIWAPLAGTCGVLLLSLVITKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 253 - protein, 211 aa, synthetic construct
IEVMYPPPYLDNEKSNGTIIHVKGKHLCPSPLFPGPSKPFWVLVVVGGVLACYSLLVTVAFIIFWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 254 - protein, 850 aa, synthetic construct
TTTPAPRPPTPAPTIASQPLSLRPEACRPAAGGAVHTRGLDFACDILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRMDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 255 - protein, 634 aa, synthetic construct
TTTPAPRPPTPAPTIASQPLSLRPEACRPAAGGAVHTRGLDFACDILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 256 - protein, 634 aa, synthetic construct
TTTPAPRPPTPAPTIASQPLSLRPEACRPAAGGAVHTRGLDFACDILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPCIVSMLRKKLVFPSD

SEQ ID NO: 257 - protein, 506 aa, synthetic construct
TTTPAPRPPTPAPTIASQPLSLRPEACRPAAGGAVHTRGLDFACDILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 258 - protein, 787 aa, synthetic construct
TTTPAPRPPTPAPTIASQPLSLRPEACRPAAGGAVHTRGLDFACDILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRMDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 259 - protein, 653 aa, synthetic construct
TTTPAPRPPTPAPTIASQPLSLRPEACRPAAGGAVHTRGLDFACDILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 260 - protein, 653 aa, synthetic construct
TTTPAPRPPTPAPTIASQPLSLRPEACRPAAGGAVHTRGLDFACDILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPCIVSMLRKKLFFKTS

SEQ ID NO: 261 - protein, 498 aa, synthetic construct
GGGGSTTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKIYIWAPLAGTCGVLLLSLVITCSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 262 - protein, 671 aa, synthetic construct
TTTPAPRPPTPAPTIASQPLSLRPEACRPAAGGAVHTRGLDFACDIYIWAPLAGTCGVLLLSLVITGGGGSGGGGSMVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 263 - protein, 526 aa, synthetic construct
TTTPAPRPPTPAPTIASQPLSLRPEACRPAAGGAVHTRGLDFACDIYIWAPLAGTCGVLLLSLVITGGGGSGGGGSMSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 264 - protein, 239 aa, synthetic construct
TTTPAPRPPTPAPTIASQPLSLRPEACRPAAGGAVHTRGLDFACDFWVLVVVGGVLACYSLLVTVAFIIFWVITQGLAVSTISSFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 265 - protein, 339 aa, synthetic construct
TTTPAPRPPTPAPTIASQPLSLRPEACRPAAGGAVHTRGLDFACDFWVLVVVGGVLACYSLLVTVAFIIFWVGGGGSGGGGSGGGGSNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERS

SEQ ID NO: 266 - DNA, 291 bp, synthetic construct
  source 1..291: mol_type = other DNA
tgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactg

SEQ ID NO: 267 - protein, 97 aa, synthetic construct
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 268 - protein, 168 aa, synthetic construct
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 269 - protein, 82 aa, synthetic construct
LTRKKKALRIHSVEGDLRRKSAGQEEWSPSAPSPPGSCVQAEAAPAGLCGEQRGEDCAELHDYFNVLSYRSLGNCSFFTETG

SEQ ID NO: 270 - protein, 41 aa, synthetic construct
AVSLSKMLKKRSPLTTGVYVKMPPTEPECEKQFQPYFIPIN

SEQ ID NO: 271 - skipped

SEQ ID NO: 272 - protein, 84 aa, synthetic construct
HRWCSNKKNAAVMDQESAGNRTANSEDSDEQDPQEVTYTQLNHCVFTQRKITRPSQRPKTPPTDIIVYTELPNAESRSKVVSCP

SEQ ID NO: 273 - protein, 145 aa, synthetic construct
KRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 274 - protein, 479 aa, synthetic construct
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 275 - protein, 263 aa, synthetic construct
SESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 276 - protein, 263 aa, synthetic construct
SESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPCIVSMLRKKLVFPSD

SEQ ID NO: 277 - protein, 135 aa, synthetic construct
GNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 278 - protein, 416 aa, synthetic construct
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 279 - protein, 282 aa, synthetic construct
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 280 - protein, 282 aa, synthetic construct
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPCIVSMLRKKLFFKTS

SEQ ID NO: 281 - protein, 595 aa, synthetic construct
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 282 - protein, 605 aa, synthetic construct
GGGGSGGGGSMVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 283 - protein, 450 aa, synthetic construct
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 284 - protein, 460 aa, synthetic construct
GGGGSGGGGSMSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 285 - protein, 13 aa, synthetic construct
ITQGLAVSTISSF

SEQ ID NO: 286 - protein, 252 aa, synthetic construct
NTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERS

SEQ ID NO: 287 - protein, 267 aa, synthetic construct
GGGGSGGGGSGGGGSNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERS

SEQ ID NO: 288 - protein, 45 aa, synthetic construct
TTTPAPRPPTPAPTIASQPLSLRPEAARPAAGGAVHTRGLDFAAD

SEQ ID NO: 289 - protein, 375 aa, synthetic construct
TTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSK

SEQ ID NO: 290 - protein, 21 aa, synthetic construct
VGVVGGLLGSLVLLVWVLAVI

SEQ ID NO: 291 - protein, 326 aa, synthetic construct
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRR

SEQ ID NO: 292 - protein, 21 aa, synthetic construct
MALPVTALLLPLALLLHAARP

SEQ ID NO: 293 - DNA, 504 bp, synthetic construct
  source 1..504: mol_type = other DNA
ctccgacatagacgccaaggcaaacactggacgtctactcaacgaaaagctgattttcagcatcccgccggcgccgtaggtcctgaaccaacggatcggggtttgcaatggcgaagtagtcctgctgctgatgcacaggaggagaacctttatgcggctgtaaaacacactcaacccgaagatggggtcgaaatggacacgcgaagcccccatgacgaagatccccaggcggtcacatatgcagaagtcaaacacagccgcccaaggcgggaaatggcgagtcccccaagcccactctcaggagagttccttgataccaaggaccgccaggccgaggaggacaggcagatggatactgaggctgccgcatcagaggcaccacaagatgttacctacgcgcaactgcactcattgacgcttcgaagagaggccaccgaacccccaccaagtcaggaaggcccgagccctgcggtcccatctatatacgcaaccctggccattcat

SEQ ID NO: 294 - DNA, 246 bp, synthetic construct
  source 1..246: mol_type = other DNA
ctcacccgaaagaagaaggctttgaggattcattccgtagaaggcgacctgcgacgcaagtcagctggtcaagaggaatggtcaccatccgcaccgtctccgcctgggtcatgtgttcaagctgaagcggctcctgccgggctttgtggtgaacaacggggcgaagattgcgccgagttgcatgattatttcaacgttcttagttaccgctctctcggaaattgtagtttttttaccgagaccgga

SEQ ID NO: 295 - DNA, 123 bp, synthetic construct
  source 1..123: mol_type = other DNA
gcagtctcactctccaaaatgctgaaaaagcgctctccccttaccaccggcgtctatgtcaaaatgccacctactgaacctgaatgcgagaagcagttccaaccttattttatccccattaat

SEQ ID NO: 296 - DNA, 12 bp, synthetic construct
  source 1..12: mol_type = other DNA
tactcatctgtt

SEQ ID NO: 297 - DNA, 252 bp, synthetic construct
  source 1..252: mol_type = other DNA
caccgatggtgttctaacaaaaagaacgcagccgttatggaccaggaaagcgcgggaaaccggacggcgaacagcgaagactccgatgagcaggatccgcaggaggtaacttacactcagcttaatcactgtgtattcactcaaaggaagatcacccggccttctcaaagaccgaaaactccgcctacggacattattgtttatacggagcttccaaacgctgagtctcggagtaaggtggtgtcttgccca

SEQ ID NO: 298 - DNA, 435 bp, synthetic construct
  source 1..435: mol_type = other DNA
aagcgaaaagaggtgcaaaagacgtgtagaaagcaccggaaggagaaccaggggtcacacgagagcccgacactgaacccagagacggttgcaataaatctttctgatgtggatttgagcaagtatatcacaacaatcgccggagtcatgacgctgagccaggttaagggctttgtacgcaagaatggcgtaaacgaggcaaaaattgacgagattaaaaatgacaacgtgcaggacaccgctgagcagaaggtacaacttttgaggaactggcatcaacttcatggaaagaaggaggcgtatgacactcttattaaggacttgaagaaggccaatctctgtacacttgcagagaaaatacaaacgattatcttgaaggatataactagtgacagtgagaactctaattttagaaatgaaatccagtctctggtc

SEQ ID NO: 299 - DNA, 1437 bp, synthetic construct
  source 1..1437: mol_type = other DNA
atggacttcagcagaaatttgtatgatatcggagaacaactcgattccgaggatcttgcgtcacttaagttcctcagcctggactacataccccagcgcaagcaagaacctataaaggacgcgctcatgttgttccaacgcctccaagagaaacgcatgctcgaagagtccaatttgagttttctcaaggaactgctgtttcggataaaccgactggatcttcttattacttatcttaatacacgcaaggaggaaatggaacgggagctgcagactcctggccgggcacagatttctgcctatagggtcatgctgtatcagatatccgaagaagtaagtcgatctgaacttcgctcatttaaattcctgttgcaagaggaaatttctaagtgcaagctggatgacgacatgaacctcctggacatattcatagaaatggaaaagcgcgtcattttgggcgaaggaaagctggatatcttgaaacgcgtctgcgctcaaataaataaatccttgttgaagattattaatgactacgaggagttttctaaagaacgctctagctctctcgaaggttcacctgatgagttttccaacggcgaggaattgtgtggagtaatgacaattagcgattccccacgggaacaagacagtgagtcccaaactctcgataaggtctaccagatgaaaagtaagcccaggggctattgcttgattatcaataaccacaactttgctaaggcccgggaaaaagtaccgaaactccactccatccgggatcgcaatggtactcacctggacgctggggcgcttactaccacctttgaagagctgcattttgagataaaaccacacgacgactgcacggttgaacaaatctatgagatattgaaaatctatcagcttatggatcattccaatatggactgtttcatctgctgcatacttagccacggggataaaggtattatatacggtacagacggtcaagaggctccgatatatgagctcacgtcacaatttactggtctcaagtgccctagtctggcagggaagcccaaggtgttcttcattcaggcctgtcagggcgataattatcagaaagggattcctgtggagacggacagtgaagagcaaccgtacctcgagatggatttgtcctctccgcagacgcgatatattccggatgaagccgatttcctgcttggcatggccactgtgaacaactgcgtctcctatcggaatcctgcagaaggtacgtggtatatacagtctctctgtcaaagtttgagggaaaggtgcccgcggggagatgatatcttgaccattttgacggaagtgaactatgaagtgagtaacaaagatgacaagaaaaacatgggtaagcagatgccgcaaccgacttttaccctgcgcaaaaaactggtcttcccttctgat

SEQ ID NO: 300 - DNA, 789 bp, synthetic construct
  source 1..789: mol_type = other DNA
agtgaaagccagacgctcgataaagtttaccagatgaagtccaaaccacggggttactgtctcataatcaataaccacaatttcgccaaggcaagagaaaaagttcctaagctccatagcatcagagacaggaacgggactcacttggacgctggggctctgactaccactttcgaggagttgcacttcgagattaaaccacatgacgactgcacagtagagcagatttacgaaatactcaaaatctatcaacttatggatcatagcaatatggactgctttatctgttgtatactttcccacggtgataagggcatcatatacgggaccgatggtcaggaagctccaatatatgaactcacaagtcagtttactggtctgaagtgtccatcattggcggggaagcccaaggtcttttttattcaggcctgtcaaggcgacaactaccaaaagggcattccagtagagacagactcagaggaacagccatatctcgagatggatctttcctcccctcaaaccagatacattccagatgaagcagacttcctgttggggatggctactgttaataactgtgtttcatatcgcaacccagcggagggaacatggtacatacagtccttgtgtcaaagtctgagggaacgatgcccaaggggggatgatatcctgactatcttgaccgaggtgaactacgaggtgtccaacaaagacgataaaaagaatatggggaaacaaatgcctcagccaacttttacgcttcgaaaaaagctcgtcttccccagcgat

SEQ ID NO: 301 - DNA, 789 bp, synthetic construct
  source 1..789: mol_type = other DNA
tcagaatcacaaactctggataaggtatatcagatgaagagcaaaccgcgaggatattgcttgattatcaacaaccataatttcgctaaagctagagaaaaagtcccaaaactgcactccattagagacaggaatggcacccatttggacgcgggtgcgctcactaccacattcgaagaattgcactttgaaattaagccccacgatgactgcactgtggaacagatttacgagatactgaaaatctatcaacttatggaccactccaacatggattgttttatatgctgcattttgtcccatggtgataagggaatcatatacggaacagatggacaggaagcgccaatttatgaacttaccagccagttcacgggacttaagtgtccgagccttgctgggaagccgaaggtcttttttatacaagcgtgtcaaggtgacaactatcagaaaggaattccagtcgaaactgattctgaagagcagccatacctggagatggatctcagttctccccagaccaggtacattcccgatgaagcggattttttgctcggtatggcaacagtgaacaactgtgtttcttacaggaacccggcagaaggtacttggtatattcaaagcttgtgccaatctcttcgggagcggtgtccgagaggagacgacatcctcactatactcacggaagtaaattatgaggtgagtaacaaggacgataaaaaaaacatgggaaaacaaatgccctgcatagtttctatgttgagaaaaaaactcgtttttcctagcgac

SEQ ID NO: 302 - DNA, 405 bp, synthetic construct
  source 1..405: mol_type = other DNA
ggcaatcgaagttctcactcacgccttggacggatcgaagcagattcagaaagtcaagaggacatcatacggaacatagcccggcatttggcccaagtaggggatagtatggaccggagtattccacctggtcttgtaaacggtttggctttgcagttgagaaacaccagtaggagtgaagaagaccgcaacagggatctcgccacggcccttgagcaacttttgcaagcatatccaagagatatggaaaaagaaaagacgatgttggtccttgcattgcttctggcaaaaaaggttgcgtctcacacgccttctttgttgcgcgatgtctttcacactacggtaaatttcatcaaccagaaccttcggacttacgtgcggtctcttgcccgaaacggcatggat

SEQ ID NO: 303 - DNA, 1248 bp, synthetic construct
  source 1..1248: mol_type = other DNA
atggacgaggctgatcgcagattgcttaggcgatgtcggttgagactggtagaagagttgcaagtcgatcaattgtgggatgcgcttctgtctagggagctgtttcggccgcacatgatcgaagacatccaaagggcaggttcaggatcacgccgagatcaagcgagacagctgataatcgatctggaaacgcgaggatcacaggcgttgcctctcttcatcagttgtctggaggatacggggcaggacatgctcgcttcctttctgcgaaccaatcgccaagccgcaaagctcagcaaaccgacacttgaaaacctgacgccggttgtgctgaggccagaaatacggaaaccagaggtcctgagacccgaaacaccacgaccagtagacattgggagcgggggtttcggagatgtaggcgcattggagtcccttcgaggcaatgcggatcttgcatatattcttagtatggaaccttgtggacactgtctcattatcaacaatgtgaatttctgccgcgagtcaggactgcgaacccggacaggctcaaacattgattgtgagaaacttcgacgcagattctcatccttgcattttatggtagaggttaagggggatcttacagcgaagaaaatggttcttgctctgctcgaacttgcacagcaagaccacggggcactggattgttgcgtagtcgtgattctttcccatggatgtcaagcgtctcatctccagtttccgggcgcggtctatggtacggacggctgccccgtttcagtagaaaagatagtcaacattttcaacggcacctcatgcccgtccttgggtggcaaacccaagttgttttttatccaagcgtgcggtggagaacagaaagatcatgggtttgaagtggcgtccacatcacccgaagatgaaagccctgggagcaaccctgaaccagacgcgactccttttcaagagggcttgcggacttttgaccagttggacgccatatcatcattgccgacgccgtctgatatattcgtctcctatagtacattccctggttttgtctcttggagggaccccaaaagcggctcttggtatgttgaaacattggatgatatttttgaacaatgggcgcacagtgaggatcttcagagcttgcttctgcgagtcgctaacgcagtttccgtaaagggaatatacaaacagatgcccggatgctttaatttcctccgcaaaaagctgttttttaagacttct

SEQ ID NO: 304 - DNA, 846 bp, synthetic construct
  source 1..846: mol_type = other DNA
ggatttggggatgtaggagcactcgaatctcttagggggaatgctgatttggcttatatcctttctatggaaccatgcggacactgtctgataataaataatgtcaatttctgccgggaaagcggattgcgaacccgaacgggctcaaacatcgattgtgagaagctgcgacgacggtttagttcccttcattttatggtcgaagtcaaaggcgatcttaccgccaagaaaatggttttggcactcttggagcttgcgcgacaagatcacggggcattggattgttgtgttgtggtcatcttgagccatggttgtcaagcgtcacacttgcagtttcccggtgccgtttatgggactgatggctgtccagtatccgttgaaaagatcgtcaatatatttaatggcacatcttgtccttccctgggcgggaagcccaaactcttttttattcaggcctgcggtggtgaacagaaagatcacggttttgaagttgcatctacctctccagaagacgaatcccctgggagcaaccctgaacctgacgcgactccattccaagaggggcttcggacgttcgaccaactcgacgcaatatcaagtttgccgacaccgagcgacatattcgtctcatacagcacattccccggtttcgtatcttggagagatccaaagtcagggagttggtatgtcgagactttggatgatatttttgaacaatgggcgcactccgaggatcttcagagtcttctcctgcgggtggcgaatgcggtgagcgtgaaaggaatttacaagcagatgccagggtgctttaacttcctccggaaaaagctgtttttcaaaactagc

SEQ ID NO: 305 - DNA, 846 bp, synthetic construct
  source 1..846: mol_type = other DNA
ggatttggtgatgtcggtgccctcgaaagtcttagaggcaacgctgatctggcgtatattttgagcatggaaccatgtggacattgtcttatcatcaataacgtaaatttttgtagggaaagtggccttaggactaggacaggttccaacatcgactgcgaaaaactgcggaggagattctcttccctccattttatggtggaagtcaaaggcgatctcaccgcaaagaagatggtactcgcattgctcgaacttgcccaacaggaccatggagctttggactgctgtgttgtagttatactgtcacatggttgccaggcaagccacctccagtttcctggggcggtatatgggaccgatggttgccccgtttccgtagagaagatagtaaatatcttcaacggaacgagctgtccgtccctcgggggcaagccaaagctttttttcattcaagcctgtggcggcgagcaaaaagaccacggattcgaggtggcatcaacgtcccctgaagatgagagtccgggcagcaatcccgaaccggatgctaccccattccaagaaggtcttaggacattcgaccaactggacgctatatctagtttgccaactccttcagatatctttgtaagttactctacgtttcctggcttcgtaagttggagagatcctaaatctggaagttggtatgtggaaacgttggatgacatttttgaacaatgggctcatagtgaagaccttcagtcactcctgcttcgcgtggcaaatgcagtgtcagttaagggtatctacaaacagatgccgtgcatcgtatccatgcttcggaaaaagctgttctttaaaacaagc

SEQ ID NO: 306 - DNA, 1785 bp, synthetic construct
  source 1..1785: mol_type = other DNA
atggtacgatggttccatcgcgacttgtccggactggacgctgagaccctcctgaaagggcggggtgtgcacggcagtttccttgcacgaccctccagaaagaatcaaggcgactttagtctttcagtaagggttggtgatcaggtcacacacattagaatccaaaattcaggtgacttctatgatttgtatggcggtgagaaatttgcaacgctcaccgaacttgttgaatattacacccagcaacagggagtacttcaggaccgcgacggtacaataattcaccttaagtaccccttgaattgttcagatcctacgtccgagagatggtatcacggacacatgagtggaggacaagctgaaaccctcctccaagcaaagggggaaccctggacttttttggtcagagagagtcttagccagccgggtgattttgttcttagtgtgctgtccgatcaaccgaaagcggggcctggttcacctttgcgcgtgacccatattaaagttatgtgtgaaggaggtcgatataccgttgggggccttgaaacattcgatagtctgacggatttggtcgaacacttcaaaaaaacagggattgaagaggcctccggcgcttttgtctatcttagacaaccctattatgcgacgagggtgaacgctgcggatatcgagaatagagttctggagctgaataaaaagcaagaatcagaggacaccgccaaagctggtttctgggaggagttcgagagtttgcagaagcaggaagttaaaaacctccaccagaggttggaagggcagagaccggaaaacaagggtaagaaccgctataaaaatatcttgcccttcgatcactctcgggtcatactgcagggtagagacagtaacattccaggcagtgattacatcaacgctaactatataaaaaatcagcttctgggcccagatgaaaatgcaaagacctatattgcgagtcagggctgtctggaggccacggttaatgacttctggcagatggcatggcaagaaaacagtagggtaatcgtcatgacaactagagaagttgagaaaggacggaacaagtgtgttccttactggcccgaggtaggcatgcagcgggcgtacgggccctacagtgttaccaactgcggcgaacatgataccacagagtataaattgcgaacactccaagtgtcaccactggataacggcgaccttatcagggagatctggcactaccaatacctgtcctggcctgatcacggcgtaccatccgaaccggggggagtgcttagctttctggatcagataaaccaaagacaggaatctctgccccatgcaggtccgatcatcgtccattgttccgcgggtataggtcggaccggaacgattatcgtaattgacatgttgatggaaaacatctctacgaaagggctcgattgcgatatagacatccagaagaccatacaaatggtacgggcacaaagatcaggcatggtccagaccgaagcccaatacaaattcatctacgtcgctattgcccagttcattgaaacaacgaaaaagaagttggaggttctccaatcccagaagggacaagagtctgagtacggtaacataacttatccgcctgccatgaaaaacgctcatgcaaaggcgagccggactagtagtaagcacaaagaagacgtttacgagaatctgcataccaaaaataagcgggaggaaaaagtaaaaaaacaacgatcagctgataaagagaaatctaaaggctcattgaagagaaaa

SEQ ID NO: 307 - DNA, 1815 bp, synthetic construct
  source 1..1815: mol_type = other DNA
ggaggtggtgggtcaggtggaggcggaagtatggtacgatggttccatcgcgacttgtccggactggacgctgagaccctcctgaaagggcggggtgtgcacggcagtttccttgcacgaccctccagaaagaatcaaggcgactttagtctttcagtaagggttggtgatcaggtcacacacattagaatccaaaattcaggtgacttctatgatttgtatggcggtgagaaatttgcaacgctcaccgaacttgttgaatattacacccagcaacagggagtacttcaggaccgcgacggtacaataattcaccttaagtaccccttgaattgttcagatcctacgtccgagagatggtatcacggacacatgagtggaggacaagctgaaaccctcctccaagcaaagggggaaccctggacttttttggtcagagagagtcttagccagccgggtgattttgttcttagtgtgctgtccgatcaaccgaaagcggggcctggttcacctttgcgcgtgacccatattaaagttatgtgtgaaggaggtcgatataccgttgggggccttgaaacattcgatagtctgacggatttggtcgaacacttcaaaaaaacagggattgaagaggcctccggcgcttttgtctatcttagacaaccctattatgcgacgagggtgaacgctgcggatatcgagaatagagttctggagctgaataaaaagcaagaatcagaggacaccgccaaagctggtttctgggaggagttcgagagtttgcagaagcaggaagttaaaaacctccaccagaggttggaagggcagagaccggaaaacaagggtaagaaccgctataaaaatatcttgcccttcgatcactctcgggtcatactgcagggtagagacagtaacattccaggcagtgattacatcaacgctaactatataaaaaatcagcttctgggcccagatgaaaatgcaaagacctatattgcgagtcagggctgtctggaggccacggttaatgacttctggcagatggcatggcaagaaaacagtagggtaatcgtcatgacaactagagaagttgagaaaggacggaacaagtgtgttccttactggcccgaggtaggcatgcagcgggcgtacgggccctacagtgttaccaactgcggcgaacatgataccacagagtataaattgcgaacactccaagtgtcaccactggataacggcgaccttatcagggagatctggcactaccaatacctgtcctggcctgatcacggcgtaccatccgaaccggggggagtgcttagctttctggatcagataaaccaaagacaggaatctctgccccatgcaggtccgatcatcgtccattgttccgcgggtataggtcggaccggaacgattatcgtaattgacatgttgatggaaaacatctctacgaaagggctcgattgcgatatagacatccagaagaccatacaaatggtacgggcacaaagatcaggcatggtccagaccgaagcccaatacaaattcatctacgtcgctattgcccagttcattgaaacaacgaaaaagaagttggaggttctccaatcccagaagggacaagagtctgagtacggtaacataacttatccgcctgccatgaaaaacgctcatgcaaaggcgagccggactagtagtaagcacaaagaagacgtttacgagaatctgcataccaaaaataagcgggaggaaaaagtaaaaaaacaacgatcagctgataaagagaaatctaaaggctcattgaagagaaaa

SEQ ID NO: 308 - DNA, 1350 bp, synthetic construct
  source 1..1350: mol_type = other DNA
atgtccgccatacaggcagcatggcccagcggaacggaatgtatcgctaaatacaactttcacggcaccgcggagcaagatcttccgttctgtaaaggtgacgttcttaccatagtggcggttactaaggacccaaattggtataaagctaagaacaaggtcgggcgcgagggcataatcccagcaaactatgtccagaagagggaaggcgtgaaagcgggaaccaaattgagcctcatgccgtggttccacggcaagattacacgcgaacaagcggagcgactgctttatccaccagagacgggtcttttcctggtccgggagtccacgaattatccgggggactatacactttgtgtaagctgtgacggcaaggtcgaacactacaggatcatgtaccacgctagtaaacttagcatagatgaggaggtatattttgagaatctcatgcagcttgtagaacattacacaagtgacgcggatggattgtgtactcgattgattaagccaaaggttatggaaggtactgtcgcagcccaagacgagttttaccgatccggctgggctctgaacatgaaagagctcaaacttctccagaccatcggtaagggtgagtttggcgacgtaatgttgggtgattaccgcggaaataaggtggctgtaaaatgtatcaagaacgatgcaacagctcaagccttcctggctgaggcgtcagtgatgacacaacttagacactcaaacttggtccaattgctcggagttatcgtcgaagagaaaggcggcctctatattgttactgagtatatggctaaaggctcccttgtggactatcttcgatctcgaggcaggtctgttctcggtggggattgcttgttgaaattcagtcttgatgtttgcgaagctatggaatatctggaaggcaacaattttgtccatagggacctggccgcccggaatgtgttggtttcagaagataacgtggccaaggtgtccgactttggtctgacaaaagaggctagctccactcaggacactgggaagttgccggtaaaatggacggcccctgaagcattgagagagaaaaaattctctactaagtccgacgtgtggtcttttggcattcttctttgggaaatctactcattcggaagggtcccgtaccctcgcattccgcttaaggacgttgtccctcgggtcgaaaagggctacaagatggacgctcctgacggatgcccgcctgcggtctacgaggtaatgaaaaattgctggcatcttgatgctgcaatgcgcccaagtttcttgcaattgcgagagcaactggaacacataaagacccacgagctccatctc

SEQ ID NO: 309 - DNA, 1380 bp, synthetic construct
  source 1..1380: mol_type = other DNA
ggaggtggtgggtcaggtggaggcggaagtatgtccgccatacaggcagcatggcccagcggaacggaatgtatcgctaaatacaactttcacggcaccgcggagcaagatcttccgttctgtaaaggtgacgttcttaccatagtggcggttactaaggacccaaattggtataaagctaagaacaaggtcgggcgcgagggcataatcccagcaaactatgtccagaagagggaaggcgtgaaagcgggaaccaaattgagcctcatgccgtggttccacggcaagattacacgcgaacaagcggagcgactgctttatccaccagagacgggtcttttcctggtccgggagtccacgaattatccgggggactatacactttgtgtaagctgtgacggcaaggtcgaacactacaggatcatgtaccacgctagtaaacttagcatagatgaggaggtatattttgagaatctcatgcagcttgtagaacattacacaagtgacgcggatggattgtgtactcgattgattaagccaaaggttatggaaggtactgtcgcagcccaagacgagttttaccgatccggctgggctctgaacatgaaagagctcaaacttctccagaccatcggtaagggtgagtttggcgacgtaatgttgggtgattaccgcggaaataaggtggctgtaaaatgtatcaagaacgatgcaacagctcaagccttcctggctgaggcgtcagtgatgacacaacttagacactcaaacttggtccaattgctcggagttatcgtcgaagagaaaggcggcctctatattgttactgagtatatggctaaaggctcccttgtggactatcttcgatctcgaggcaggtctgttctcggtggggattgcttgttgaaattcagtcttgatgtttgcgaagctatggaatatctggaaggcaacaattttgtccatagggacctggccgcccggaatgtgttggtttcagaagataacgtggccaaggtgtccgactttggtctgacaaaagaggctagctccactcaggacactgggaagttgccggtaaaatggacggcccctgaagcattgagagagaaaaaattctctactaagtccgacgtgtggtcttttggcattcttctttgggaaatctactcattcggaagggtcccgtaccctcgcattccgcttaaggacgttgtccctcgggtcgaaaagggctacaagatggacgctcctgacggatgcccgcctgcggtctacgaggtaatgaaaaattgctggcatcttgatgctgcaatgcgcccaagtttcttgcaattgcgagagcaactggaacacataaagacccacgagctccatctc

SEQ ID NO: 310 - DNA, 39 bp, synthetic construct
  source 1..39: mol_type = other DNA
atcacccagggcctggccgtgagcaccatcagcagcttc

SEQ ID NO: 311 - DNA, 126 bp, synthetic construct
  source 1..126: mol_type = other DNA
aaacggggcagaaagaaactcctgtatatattcaaacaaccatttatgcgaccagtacaaactactcaagaggaagatggctgtagctgccgatttccagaagaagaagaaggaggatgtgaactg

SEQ ID NO: 312 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctaa

SEQ ID NO: 313 - DNA, 756 bp, synthetic construct
  source 1..756: mol_type = other DNA
aatacgtgtaagctccttgtggtagcagaccataggttctacagatatatgggacgaggagaggaaagcactacgacaaactatttgatcgaacttatagatagagtcgacgacatctaccgaaacacgtcatgggacaatgcaggcttcaagggatacggaatacagatagagcagatacggattctgaaaagtccgcaggaagtgaaaccgggggagaaacattataatatggcgaaatcatatccaaacgaggagaaagatgcgtgggatgtaaaaatgctcttggaacagtttagttttgacattgctgaggaagccagcaaagtgtgcctcgctcacctgtttacttatcaagattttgatatgggcaccctgggtcttgcatatgtagggtctcccagagcaaactcccacgggggcgtatgtccaaaagcttactacagcccagtaggaaaaaagaatatatacctgaacagcgggctgactagtaccaaaaattacggaaaaactattctcacgaaggaggccgacttggtaactactcatgaactggggcataacttcggcgccgagcacgacccggacggacttgcagagtgcgccccgaacgaggaccaaggtggcaaatatgtcatgtacccaatcgccgtttcaggggatcatgagaacaataagatgttcagcaactgttccaaacaatccatttataaaacgatagaaagcaaagcccaggaatgttttcaggaaagatca

SEQ ID NO: 314 - DNA, 801 bp, synthetic construct
  source 1..801: mol_type = other DNA
ggcggcgggggatctggaggtggtggcagcggtgggggcggtagtaatacgtgtaagctccttgtggtagcagaccataggttctacagatatatgggacgaggagaggaaagcactacgacaaactatttgatcgaacttatagatagagtcgacgacatctaccgaaacacgtcatgggacaatgcaggcttcaagggatacggaatacagatagagcagatacggattctgaaaagtccgcaggaagtgaaaccgggggagaaacattataatatggcgaaatcatatccaaacgaggagaaagatgcgtgggatgtaaaaatgctcttggaacagtttagttttgacattgctgaggaagccagcaaagtgtgcctcgctcacctgtttacttatcaagattttgatatgggcaccctgggtcttgcatatgtagggtctcccagagcaaactcccacgggggcgtatgtccaaaagcttactacagcccagtaggaaaaaagaatatatacctgaacagcgggctgactagtaccaaaaattacggaaaaactattctcacgaaggaggccgacttggtaactactcatgaactggggcataacttcggcgccgagcacgacccggacggacttgcagagtgcgccccgaacgaggaccaaggtggcaaatatgtcatgtacccaatcgccgtttcaggggatcatgagaacaataagatgttcagcaactgttccaaacaatccatttataaaacgatagaaagcaaagcccaggaatgttttcaggaaagatca

SEQ ID NO: 315 - DNA, 135 bp, synthetic construct
  source 1..135: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgac

SEQ ID NO: 316 - DNA, 135 bp, synthetic construct
  source 1..135: mol_type = other DNA
actaccaccccagcacccagaccgccaactccggctcctacgatagcgtcccaacctctctctcttaggccagaggcagctcgcccggcagctgggggtgcggttcatactcggggacttgatttcgctgctgac

SEQ ID NO: 317 - DNA, 117 bp, synthetic construct
  source 1..117: mol_type = other DNA
atcgaagtgatgtaccctccaccttacctggacaacgagaagtccaacggcaccatcatccacgtgaagggcaagcacctgtgtccttctccactgttccccggacctagcaagcct

SEQ ID NO: 318 - DNA, 1125 bp, synthetic construct
  source 1..1125: mol_type = other DNA
acaacgaccttatctccatccggttccgcagtcattagcaccacgaccatagctaccacgccaagtaagccaacttgcgatgagaaatacgctaacatcaccgtggattatctgtataataaagaaacgaagttgttcacagctaagctcaatgtaaacgagaacgtggagtgcgggaataatacttgcactaacaacgaagtccataatctcactgagtgcaagaatgcttctgtgtccatttctcataactcttgcactgccccagataagacactgatactggatgtgccaccaggcgtggaaaagtttcagcttcacgactgtacccaggtcgagaaagctgacacgactatctgtctgaaatggaagaacatagaaacgtttacatgcgatacacagaacattacctatcgattccaatgcgggaacatgattttcgacaataaggaaattaaactggagaatctggagcctgaacatgagtacaaatgtgactccgaaatcctgtacaataatcacaagttcacaaatgcgagcaaaattatcaagactgacttcggatcaccaggcgaaccccagattattttctgtcgcagtgaggctgcacatcagggagtcataacatggaacccgccacagagaagtttccataactttacactctgttacataaaggagactgagaaagattgccttaacttagataagaacctgatcaagtacgaccttcagaacttgaaaccgtataccaagtatgtcctttccctgcacgcctacataatcgccaaagtgcagcgaaatggatctgctgccatgtgccatttcactacgaaatctgcaccaccttcccaggtgtggaacatgacagtgtctatgaccagtgacaattcaatgcacgttaaatgtagaccaccgagggatagaaatggtccacacgagaggtaccatttagaggtagaggcaggaaataccttagtgagaaatgagagccacaagaactgcgacttcagagtgaaagacttacagtactccactgattatacatttaaagcttatttccataatggagattaccccggcgaaccttttatactccaccactctactagctacaactccaaa

SEQ ID NO: 319 - protein, 380 aa, synthetic construct
GGGGSTTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSK

SEQ ID NO: 320 - DNA, 1140 bp, synthetic construct
  source 1..1140: mol_type = other DNA
ggtggaggcggttccacaacgaccttatctccatccggttccgcagtcattagcaccacgaccatagctaccacgccaagtaagccaacttgcgatgagaaatacgctaacatcaccgtggattatctgtataataaagaaacgaagttgttcacagctaagctcaatgtaaacgagaacgtggagtgcgggaataatacttgcactaacaacgaagtccataatctcactgagtgcaagaatgcttctgtgtccatttctcataactcttgcactgccccagataagacactgatactggatgtgccaccaggcgtggaaaagtttcagcttcacgactgtacccaggtcgagaaagctgacacgactatctgtctgaaatggaagaacatagaaacgtttacatgcgatacacagaacattacctatcgattccaatgcgggaacatgattttcgacaataaggaaattaaactggagaatctggagcctgaacatgagtacaaatgtgactccgaaatcctgtacaataatcacaagttcacaaatgcgagcaaaattatcaagactgacttcggatcaccaggcgaaccccagattattttctgtcgcagtgaggctgcacatcagggagtcataacatggaacccgccacagagaagtttccataactttacactctgttacataaaggagactgagaaagattgccttaacttagataagaacctgatcaagtacgaccttcagaacttgaaaccgtataccaagtatgtcctttccctgcacgcctacataatcgccaaagtgcagcgaaatggatctgctgccatgtgccatttcactacgaaatctgcaccaccttcccaggtgtggaacatgacagtgtctatgaccagtgacaattcaatgcacgttaaatgtagaccaccgagggatagaaatggtccacacgagaggtaccatttagaggtagaggcaggaaataccttagtgagaaatgagagccacaagaactgcgacttcagagtgaaagacttacagtactccactgattatacatttaaagcttatttccataatggagattaccccggcgaaccttttatactccaccactctactagctacaactccaaa

SEQ ID NO: 321 - protein, 154 aa, synthetic construct
VLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVH

SEQ ID NO: 322 - DNA, 462 bp, synthetic construct
  source 1..462: mol_type = other DNA
gtccttgaggtgtccgaccaccaggtcctgaacgatgctgaggtggcagcgctgttggagaacttcagttcatcctacgattacggagagaacgagtctgatagttgttgtaccagtcccccctgcccccaggatttttcccttaatttcgacagggctttcctccccgcactgtattcccttctgtttcttttagggctgttaggcaatggcgccgtggcggctgtccttctgagtcggcgcacagccctctccagtacggatacctttctcctgcacctggcggtggccgacacgctgttggtcctgactcttccactctgggccgttgatgccgcagtccaatgggtttttggctccggtctgtgcaaggtagcgggagctctgttcaatatcaacttctacgctggagctctgctgctggcctgcatcagcttcgatagatatctgaacatcgtccat

SEQ ID NO: 323 - protein, 79 aa, synthetic construct
PARVTLTCLAVWGLCLLFALPDFIFLSAHHDERLNATHCQYNFPQVGRTALRVLQLVAGFLLPLLVMAYCYAHILAVLL

SEQ ID NO: 324 - DNA, 240 bp, synthetic construct
  source 1..240: mol_type = other DNA
ccagccagagtaacactgacatgtctcgctgtctggggtctgtgcctgcttttcgccctgcctgacttcattttcctgtctgctcaccacgacgagcgtctgaatgccacccactgccagtataatttccctcaagtaggtcggaccgcccttcgagttttgcagctggtggccggtttcttattgcccttactggtgatggcttactgctacgcacacatcttggctgtgcttctcgtt

SEQ ID NO: 325 - protein, 74 aa, synthetic construct
QRRLRAMRLVVVVVVAFALCWTPYHLVVLVDILMDLGALARNCGRESRVDVAKSVTSGLGYMHCCLNPLLYAFV

SEQ ID NO: 326 - DNA, 222 bp, synthetic construct
  source 1..222: mol_type = other DNA
cagaggaggctgagggccatgagactcgttgtcgtcgtggttgttgctttcgccttatgctggaccccataccatttagttgtgctggtggatatcctgatggacctgggcgccctcgcacggaactgtggcagggaatcaagagtggacgtcgctaaatccgtgacctccggactgggttatatgcactgttgcctcaaccctctgctctacgcctttgtg

SEQ ID NO: 327 - DNA, 63 bp, synthetic construct
  source 1..63: mol_type = other DNA
atggcccttcccgtgactgcgctcctcctgcctcttgccctccttcttcatgctgctcgcccg

SEQ ID NO: 328 - DNA, 489 bp, synthetic construct
  source 1..489: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattacctgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactg

SEQ ID NO: 329 - DNA, 489 bp, synthetic construct
  source 1..489: mol_type = other DNA
actaccaccccagcacccagaccgccaactccggctcctacgatagcgtcccaacctctctctcttaggccagaggcagctcgcccggcagctgggggtgcggttcatactcggggacttgatttcgctgctgacgtgggagttgttgggggacttcttggatctcttgtccttcttgtatgggtgttggcagtcatatgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactg

SEQ ID NO: 330 - DNA, 489 bp, synthetic construct
  source 1..489: mol_type = other DNA
atcgaagtgatgtaccctccaccttacctggacaacgagaagtccaacggcaccatcatccacgtgaagggcaagcacctgtgtccttctccactgttccccggacctagcaagcctttttgggtgctggtggtggttggtggagtcctggcttgctattccttgctagtaacagtggccttcatcatcttttgggtctgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactg

SEQ ID NO: 331 - DNA, 507 bp, synthetic construct
  source 1..507: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacttttgggtgctggtggtggttggtggagtcctggcttgctattccttgctagtaacagtggccttcatcatcttttgggtctgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactg

SEQ ID NO: 332 - DNA, 702 bp, synthetic construct
  source 1..702: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattaccctccgacatagacgccaaggcaaacactggacgtctactcaacgaaaagctgattttcagcatcccgccggcgccgtaggtcctgaaccaacggatcggggtttgcaatggcgaagtagtcctgctgctgatgcacaggaggagaacctttatgcggctgtaaaacacactcaacccgaagatggggtcgaaatggacacgcgaagcccccatgacgaagatccccaggcggtcacatatgcagaagtcaaacacagccgcccaaggcgggaaatggcgagtcccccaagcccactctcaggagagttccttgataccaaggaccgccaggccgaggaggacaggcagatggatactgaggctgccgcatcagaggcaccacaagatgttacctacgcgcaactgcactcattgacgcttcgaagagaggccaccgaacccccaccaagtcaggaaggcccgagccctgcggtcccatctatatacgcaaccctggccattcat

SEQ ID NO: 333 - DNA, 735 bp, synthetic construct
  source 1..735: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattacctgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactgctcacccgaaagaagaaggctttgaggattcattccgtagaaggcgacctgcgacgcaagtcagctggtcaagaggaatggtcaccatccgcaccgtctccgcctgggtcatgtgttcaagctgaagcggctcctgccgggctttgtggtgaacaacggggcgaagattgcgccgagttgcatgattatttcaacgttcttagttaccgctctctcggaaattgtagtttttttaccgagaccgga

SEQ ID NO: 334 - DNA, 612 bp, synthetic construct
  source 1..612: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattacctgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactggcagtctcactctccaaaatgctgaaaaagcgctctccccttaccaccggcgtctatgtcaaaatgccacctactgaacctgaatgcgagaagcagttccaaccttattttatccccattaat

SEQ ID NO: 335 - DNA, 624 bp, synthetic construct
  source 1..624: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattacctgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactggcagtctcactctccaaaatgctgaaaaagcgctctccccttaccaccggcgtctatgtcaaaatgccacctactgaacctgaatgcgagaagcagttccaaccttattttatccccattaattactcatctgtt

SEQ ID NO: 336 - DNA, 501 bp, synthetic construct
  source 1..501: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattacctgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactgtactcatctgtt

SEQ ID NO: 337 - DNA, 450 bp, synthetic construct
  source 1..450: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattacccaccgatggtgttctaacaaaaagaacgcagccgttatggaccaggaaagcgcgggaaaccggacggcgaacagcgaagactccgatgagcaggatccgcaggaggtaacttacactcagcttaatcactgtgtattcactcaaaggaagatcacccggccttctcaaagaccgaaaactccgcctacggacattattgtttatacggagcttccaaacgctgagtctcggagtaaggtggtgtcttgccca

SEQ ID NO: 338 - DNA, 633 bp, synthetic construct
  source 1..633: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattaccaagcgaaaagaggtgcaaaagacgtgtagaaagcaccggaaggagaaccaggggtcacacgagagcccgacactgaacccagagacggttgcaataaatctttctgatgtggatttgagcaagtatatcacaacaatcgccggagtcatgacgctgagccaggttaagggctttgtacgcaagaatggcgtaaacgaggcaaaaattgacgagattaaaaatgacaacgtgcaggacaccgctgagcagaaggtacaacttttgaggaactggcatcaacttcatggaaagaaggaggcgtatgacactcttattaaggacttgaagaaggccaatctctgtacacttgcagagaaaatacaaacgattatcttgaaggatataactagtgacagtgagaactctaattttagaaatgaaatccagtctctggtc

SEQ ID NO: 339 - DNA, 633 bp, synthetic construct
  source 1..633: mol_type = other DNA
atcgaagtgatgtaccctccaccttacctggacaacgagaagtccaacggcaccatcatccacgtgaagggcaagcacctgtgtccttctccactgttccccggacctagcaagcctttttgggtgctggtggtggttggtggagtcctggcttgctattccttgctagtaacagtggccttcatcatcttttgggtcaagcgaaaagaggtgcaaaagacgtgtagaaagcaccggaaggagaaccaggggtcacacgagagcccgacactgaacccagagacggttgcaataaatctttctgatgtggatttgagcaagtatatcacaacaatcgccggagtcatgacgctgagccaggttaagggctttgtacgcaagaatggcgtaaacgaggcaaaaattgacgagattaaaaatgacaacgtgcaggacaccgctgagcagaaggtacaacttttgaggaactggcatcaacttcatggaaagaaggaggcgtatgacactcttattaaggacttgaagaaggccaatctctgtacacttgcagagaaaatacaaacgattatcttgaaggatataactagtgacagtgagaactctaattttagaaatgaaatccagtctctggtc

SEQ ID NO: 340 - DNA, 2550 bp, synthetic construct
  source 1..2550: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacattttggattatagtttcaccggaggcgccggacgcgacatacctcccccgcaaatcgaggaagcgtgcgaactcccagagtgtcaagtagacgccggcaacaaggtgtgcaacctgcaatgcaacaaccatgcctgcggatgggatggaggggactgtagtttgaatttcaacgacccttggaagaactgcacgcagtctcttcaatgctggaaatatttcagcgatggacattgtgacagccagtgtaacagcgccggatgcttgtttgatggctttgattgtcaattgaccgagggtcagtgcaacccgctctatgaccaatattgtaaggatcacttttccgacgggcactgcgatcagggctgcaatagcgcggaatgtgaatgggacggattggattgtgcagaacatgtgcccgaacgcctcgcggcaggaactctggtcctcgtagtcttgttgccgcccgaccaattgcggaacaacagtttccactttctgcgggaattgtcacatgtgctgcatacaaatgtcgtgtttaagcgcgatgctcagggtcaacaaatgatattcccgtactatggtcacgaagaggagcttaggaagcacccgattaagcggtctacggttggatgggctacttcctctcttctcccggggacgagcgggggcagacaaaggcgggagctcgacccaatggacattagaggatcaatagtgtacctggagattgacaatcggcaatgcgtccaatctagcagtcaatgctttcagtctgcaaccgatgtcgcagcctttctgggcgccctggccagcctcgggtcccttaatatcccgtataagattgaggcggttaaatctgaacctgtcgaacctcccctcccctctcaattgcacctcatgtacgttgctgctgctgcctttgttctgctcttcttcgtaggatgcggggttcttctctccagaaaacgacggcgcatggacttcagcagaaatttgtatgatatcggagaacaactcgattccgaggatcttgcgtcacttaagttcctcagcctggactacataccccagcgcaagcaagaacctataaaggacgcgctcatgttgttccaacgcctccaagagaaacgcatgctcgaagagtccaatttgagttttctcaaggaactgctgtttcggataaaccgactggatcttcttattacttatcttaatacacgcaaggaggaaatggaacgggagctgcagactcctggccgggcacagatttctgcctatagggtcatgctgtatcagatatccgaagaagtaagtcgatctgaacttcgctcatttaaattcctgttgcaagaggaaatttctaagtgcaagctggatgacgacatgaacctcctggacatattcatagaaatggaaaagcgcgtcattttgggcgaaggaaagctggatatcttgaaacgcgtctgcgctcaaataaataaatccttgttgaagattattaatgactacgaggagttttctaaagaacgctctagctctctcgaaggttcacctgatgagttttccaacggcgaggaattgtgtggagtaatgacaattagcgattccccacgggaacaagacagtgagtcccaaactctcgataaggtctaccagatgaaaagtaagcccaggggctattgcttgattatcaataaccacaactttgctaaggcccgggaaaaagtaccgaaactccactccatccgggatcgcaatggtactcacctggacgctggggcgcttactaccacctttgaagagctgcattttgagataaaaccacacgacgactgcacggttgaacaaatctatgagatattgaaaatctatcagcttatggatcattccaatatggactgtttcatctgctgcatacttagccacggggataaaggtattatatacggtacagacggtcaagaggctccgatatatgagctcacgtcacaatttactggtctcaagtgccctagtctggcagggaagcccaaggtgttcttcattcaggcctgtcagggcgataattatcagaaagggattcctgtggagacggacagtgaagagcaaccgtacctcgagatggatttgtcctctccgcagacgcgatatattccggatgaagccgatttcctgcttggcatggccactgtgaacaactgcgtctcctatcggaatcctgcagaaggtacgtggtatatacagtctctctgtcaaagtttgagggaaaggtgcccgcggggagatgatatcttgaccattttgacggaagtgaactatgaagtgagtaacaaagatgacaagaaaaacatgggtaagcagatgccgcaaccgacttttaccctgcgcaaaaaactggtcttcccttctgat

SEQ ID NO: 341 - DNA, 1902 bp, synthetic construct
  source 1..1902: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacattttggattatagtttcaccggaggcgccggacgcgacatacctcccccgcaaatcgaggaagcgtgcgaactcccagagtgtcaagtagacgccggcaacaaggtgtgcaacctgcaatgcaacaaccatgcctgcggatgggatggaggggactgtagtttgaatttcaacgacccttggaagaactgcacgcagtctcttcaatgctggaaatatttcagcgatggacattgtgacagccagtgtaacagcgccggatgcttgtttgatggctttgattgtcaattgaccgagggtcagtgcaacccgctctatgaccaatattgtaaggatcacttttccgacgggcactgcgatcagggctgcaatagcgcggaatgtgaatgggacggattggattgtgcagaacatgtgcccgaacgcctcgcggcaggaactctggtcctcgtagtcttgttgccgcccgaccaattgcggaacaacagtttccactttctgcgggaattgtcacatgtgctgcatacaaatgtcgtgtttaagcgcgatgctcagggtcaacaaatgatattcccgtactatggtcacgaagaggagcttaggaagcacccgattaagcggtctacggttggatgggctacttcctctcttctcccggggacgagcgggggcagacaaaggcgggagctcgacccaatggacattagaggatcaatagtgtacctggagattgacaatcggcaatgcgtccaatctagcagtcaatgctttcagtctgcaaccgatgtcgcagcctttctgggcgccctggccagcctcgggtcccttaatatcccgtataagattgaggcggttaaatctgaacctgtcgaacctcccctcccctctcaattgcacctcatgtacgttgctgctgctgcctttgttctgctcttcttcgtaggatgcggggttcttctctccagaaaacgacggcgcagtgaaagccagacgctcgataaagtttaccagatgaagtccaaaccacggggttactgtctcataatcaataaccacaatttcgccaaggcaagagaaaaagttcctaagctccatagcatcagagacaggaacgggactcacttggacgctggggctctgactaccactttcgaggagttgcacttcgagattaaaccacatgacgactgcacagtagagcagatttacgaaatactcaaaatctatcaacttatggatcatagcaatatggactgctttatctgttgtatactttcccacggtgataagggcatcatatacgggaccgatggtcaggaagctccaatatatgaactcacaagtcagtttactggtctgaagtgtccatcattggcggggaagcccaaggtcttttttattcaggcctgtcaaggcgacaactaccaaaagggcattccagtagagacagactcagaggaacagccatatctcgagatggatctttcctcccctcaaaccagatacattccagatgaagcagacttcctgttggggatggctactgttaataactgtgtttcatatcgcaacccagcggagggaacatggtacatacagtccttgtgtcaaagtctgagggaacgatgcccaaggggggatgatatcctgactatcttgaccgaggtgaactacgaggtgtccaacaaagacgataaaaagaatatggggaaacaaatgcctcagccaacttttacgcttcgaaaaaagctcgtcttccccagcgat

SEQ ID NO: 342 - DNA, 1902 bp, synthetic construct
  source 1..1902: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacattttggattatagtttcaccggaggcgccggacgcgacatacctcccccgcaaatcgaggaagcgtgcgaactcccagagtgtcaagtagacgccggcaacaaggtgtgcaacctgcaatgcaacaaccatgcctgcggatgggatggaggggactgtagtttgaatttcaacgacccttggaagaactgcacgcagtctcttcaatgctggaaatatttcagcgatggacattgtgacagccagtgtaacagcgccggatgcttgtttgatggctttgattgtcaattgaccgagggtcagtgcaacccgctctatgaccaatattgtaaggatcacttttccgacgggcactgcgatcagggctgcaatagcgcggaatgtgaatgggacggattggattgtgcagaacatgtgcccgaacgcctcgcggcaggaactctggtcctcgtagtcttgttgccgcccgaccaattgcggaacaacagtttccactttctgcgggaattgtcacatgtgctgcatacaaatgtcgtgtttaagcgcgatgctcagggtcaacaaatgatattcccgtactatggtcacgaagaggagcttaggaagcacccgattaagcggtctacggttggatgggctacttcctctcttctcccggggacgagcgggggcagacaaaggcgggagctcgacccaatggacattagaggatcaatagtgtacctggagattgacaatcggcaatgcgtccaatctagcagtcaatgctttcagtctgcaaccgatgtcgcagcctttctgggcgccctggccagcctcgggtcccttaatatcccgtataagattgaggcggttaaatctgaacctgtcgaacctcccctcccctctcaattgcacctcatgtacgttgctgctgctgcctttgttctgctcttcttcgtaggatgcggggttcttctctccagaaaacgacggcgctcagaatcacaaactctggataaggtatatcagatgaagagcaaaccgcgaggatattgcttgattatcaacaaccataatttcgctaaagctagagaaaaagtcccaaaactgcactccattagagacaggaatggcacccatttggacgcgggtgcgctcactaccacattcgaagaattgcactttgaaattaagccccacgatgactgcactgtggaacagatttacgagatactgaaaatctatcaacttatggaccactccaacatggattgttttatatgctgcattttgtcccatggtgataagggaatcatatacggaacagatggacaggaagcgccaatttatgaacttaccagccagttcacgggacttaagtgtccgagccttgctgggaagccgaaggtcttttttatacaagcgtgtcaaggtgacaactatcagaaaggaattccagtcgaaactgattctgaagagcagccatacctggagatggatctcagttctccccagaccaggtacattcccgatgaagcggattttttgctcggtatggcaacagtgaacaactgtgtttcttacaggaacccggcagaaggtacttggtatattcaaagcttgtgccaatctcttcgggagcggtgtccgagaggagacgacatcctcactatactcacggaagtaaattatgaggtgagtaacaaggacgataaaaaaaacatgggaaaacaaatgccctgcatagtttctatgttgagaaaaaaactcgtttttcctagcgac

SEQ ID NO: 343 - DNA, 1518 bp, synthetic construct
  source 1..1518: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacattttggattatagtttcaccggaggcgccggacgcgacatacctcccccgcaaatcgaggaagcgtgcgaactcccagagtgtcaagtagacgccggcaacaaggtgtgcaacctgcaatgcaacaaccatgcctgcggatgggatggaggggactgtagtttgaatttcaacgacccttggaagaactgcacgcagtctcttcaatgctggaaatatttcagcgatggacattgtgacagccagtgtaacagcgccggatgcttgtttgatggctttgattgtcaattgaccgagggtcagtgcaacccgctctatgaccaatattgtaaggatcacttttccgacgggcactgcgatcagggctgcaatagcgcggaatgtgaatgggacggattggattgtgcagaacatgtgcccgaacgcctcgcggcaggaactctggtcctcgtagtcttgttgccgcccgaccaattgcggaacaacagtttccactttctgcgggaattgtcacatgtgctgcatacaaatgtcgtgtttaagcgcgatgctcagggtcaacaaatgatattcccgtactatggtcacgaagaggagcttaggaagcacccgattaagcggtctacggttggatgggctacttcctctcttctcccggggacgagcgggggcagacaaaggcgggagctcgacccaatggacattagaggatcaatagtgtacctggagattgacaatcggcaatgcgtccaatctagcagtcaatgctttcagtctgcaaccgatgtcgcagcctttctgggcgccctggccagcctcgggtcccttaatatcccgtataagattgaggcggttaaatctgaacctgtcgaacctcccctcccctctcaattgcacctcatgtacgttgctgctgctgcctttgttctgctcttcttcgtaggatgcggggttcttctctccagaaaacgacggcgcggcaatcgaagttctcactcacgccttggacggatcgaagcagattcagaaagtcaagaggacatcatacggaacatagcccggcatttggcccaagtaggggatagtatggaccggagtattccacctggtcttgtaaacggtttggctttgcagttgagaaacaccagtaggagtgaagaagaccgcaacagggatctcgccacggcccttgagcaacttttgcaagcatatccaagagatatggaaaaagaaaagacgatgttggtccttgcattgcttctggcaaaaaaggttgcgtctcacacgccttctttgttgcgcgatgtctttcacactacggtaaatttcatcaaccagaaccttcggacttacgtgcggtctcttgcccgaaacggcatggat

SEQ ID NO: 344 - DNA, 2361 bp, synthetic construct
  source 1..2361: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacattttggattatagtttcaccggaggcgccggacgcgacatacctcccccgcaaatcgaggaagcgtgcgaactcccagagtgtcaagtagacgccggcaacaaggtgtgcaacctgcaatgcaacaaccatgcctgcggatgggatggaggggactgtagtttgaatttcaacgacccttggaagaactgcacgcagtctcttcaatgctggaaatatttcagcgatggacattgtgacagccagtgtaacagcgccggatgcttgtttgatggctttgattgtcaattgaccgagggtcagtgcaacccgctctatgaccaatattgtaaggatcacttttccgacgggcactgcgatcagggctgcaatagcgcggaatgtgaatgggacggattggattgtgcagaacatgtgcccgaacgcctcgcggcaggaactctggtcctcgtagtcttgttgccgcccgaccaattgcggaacaacagtttccactttctgcgggaattgtcacatgtgctgcatacaaatgtcgtgtttaagcgcgatgctcagggtcaacaaatgatattcccgtactatggtcacgaagaggagcttaggaagcacccgattaagcggtctacggttggatgggctacttcctctcttctcccggggacgagcgggggcagacaaaggcgggagctcgacccaatggacattagaggatcaatagtgtacctggagattgacaatcggcaatgcgtccaatctagcagtcaatgctttcagtctgcaaccgatgtcgcagcctttctgggcgccctggccagcctcgggtcccttaatatcccgtataagattgaggcggttaaatctgaacctgtcgaacctcccctcccctctcaattgcacctcatgtacgttgctgctgctgcctttgttctgctcttcttcgtaggatgcggggttcttctctccagaaaacgacggcgcatggacgaggctgatcgcagattgcttaggcgatgtcggttgagactggtagaagagttgcaagtcgatcaattgtgggatgcgcttctgtctagggagctgtttcggccgcacatgatcgaagacatccaaagggcaggttcaggatcacgccgagatcaagcgagacagctgataatcgatctggaaacgcgaggatcacaggcgttgcctctcttcatcagttgtctggaggatacggggcaggacatgctcgcttcctttctgcgaaccaatcgccaagccgcaaagctcagcaaaccgacacttgaaaacctgacgccggttgtgctgaggccagaaatacggaaaccagaggtcctgagacccgaaacaccacgaccagtagacattgggagcgggggtttcggagatgtaggcgcattggagtcccttcgaggcaatgcggatcttgcatatattcttagtatggaaccttgtggacactgtctcattatcaacaatgtgaatttctgccgcgagtcaggactgcgaacccggacaggctcaaacattgattgtgagaaacttcgacgcagattctcatccttgcattttatggtagaggttaagggggatcttacagcgaagaaaatggttcttgctctgctcgaacttgcacagcaagaccacggggcactggattgttgcgtagtcgtgattctttcccatggatgtcaagcgtctcatctccagtttccgggcgcggtctatggtacggacggctgccccgtttcagtagaaaagatagtcaacattttcaacggcacctcatgcccgtccttgggtggcaaacccaagttgttttttatccaagcgtgcggtggagaacagaaagatcatgggtttgaagtggcgtccacatcacccgaagatgaaagccctgggagcaaccctgaaccagacgcgactccttttcaagagggcttgcggacttttgaccagttggacgccatatcatcattgccgacgccgtctgatatattcgtctcctatagtacattccctggttttgtctcttggagggaccccaaaagcggctcttggtatgttgaaacattggatgatatttttgaacaatgggcgcacagtgaggatcttcagagcttgcttctgcgagtcgctaacgcagtttccgtaaagggaatatacaaacagatgcccggatgctttaatttcctccgcaaaaagctgttttttaagacttct

SEQ ID NO: 345 - DNA, 1959 bp, synthetic construct
  source 1..1959: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacattttggattatagtttcaccggaggcgccggacgcgacatacctcccccgcaaatcgaggaagcgtgcgaactcccagagtgtcaagtagacgccggcaacaaggtgtgcaacctgcaatgcaacaaccatgcctgcggatgggatggaggggactgtagtttgaatttcaacgacccttggaagaactgcacgcagtctcttcaatgctggaaatatttcagcgatggacattgtgacagccagtgtaacagcgccggatgcttgtttgatggctttgattgtcaattgaccgagggtcagtgcaacccgctctatgaccaatattgtaaggatcacttttccgacgggcactgcgatcagggctgcaatagcgcggaatgtgaatgggacggattggattgtgcagaacatgtgcccgaacgcctcgcggcaggaactctggtcctcgtagtcttgttgccgcccgaccaattgcggaacaacagtttccactttctgcgggaattgtcacatgtgctgcatacaaatgtcgtgtttaagcgcgatgctcagggtcaacaaatgatattcccgtactatggtcacgaagaggagcttaggaagcacccgattaagcggtctacggttggatgggctacttcctctcttctcccggggacgagcgggggcagacaaaggcgggagctcgacccaatggacattagaggatcaatagtgtacctggagattgacaatcggcaatgcgtccaatctagcagtcaatgctttcagtctgcaaccgatgtcgcagcctttctgggcgccctggccagcctcgggtcccttaatatcccgtataagattgaggcggttaaatctgaacctgtcgaacctcccctcccctctcaattgcacctcatgtacgttgctgctgctgcctttgttctgctcttcttcgtaggatgcggggttcttctctccagaaaacgacggcgcggatttggggatgtaggagcactcgaatctcttagggggaatgctgatttggcttatatcctttctatggaaccatgcggacactgtctgataataaataatgtcaatttctgccgggaaagcggattgcgaacccgaacgggctcaaacatcgattgtgagaagctgcgacgacggtttagttcccttcattttatggtcgaagtcaaaggcgatcttaccgccaagaaaatggttttggcactcttggagcttgcgcgacaagatcacggggcattggattgttgtgttgtggtcatcttgagccatggttgtcaagcgtcacacttgcagtttcccggtgccgtttatgggactgatggctgtccagtatccgttgaaaagatcgtcaatatatttaatggcacatcttgtccttccctgggcgggaagcccaaactcttttttattcaggcctgcggtggtgaacagaaagatcacggttttgaagttgcatctacctctccagaagacgaatcccctgggagcaaccctgaacctgacgcgactccattccaagaggggcttcggacgttcgaccaactcgacgcaatatcaagtttgccgacaccgagcgacatattcgtctcatacagcacattccccggtttcgtatcttggagagatccaaagtcagggagttggtatgtcgagactttggatgatatttttgaacaatgggcgcactccgaggatcttcagagtcttctcctgcgggtggcgaatgcggtgagcgtgaaaggaatttacaagcagatgccagggtgctttaacttcctccggaaaaagctgtttttcaaaactagc

SEQ ID NO: 346 - DNA, 1959 bp, synthetic construct
  source 1..1959: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacattttggattatagtttcaccggaggcgccggacgcgacatacctcccccgcaaatcgaggaagcgtgcgaactcccagagtgtcaagtagacgccggcaacaaggtgtgcaacctgcaatgcaacaaccatgcctgcggatgggatggaggggactgtagtttgaatttcaacgacccttggaagaactgcacgcagtctcttcaatgctggaaatatttcagcgatggacattgtgacagccagtgtaacagcgccggatgcttgtttgatggctttgattgtcaattgaccgagggtcagtgcaacccgctctatgaccaatattgtaaggatcacttttccgacgggcactgcgatcagggctgcaatagcgcggaatgtgaatgggacggattggattgtgcagaacatgtgcccgaacgcctcgcggcaggaactctggtcctcgtagtcttgttgccgcccgaccaattgcggaacaacagtttccactttctgcgggaattgtcacatgtgctgcatacaaatgtcgtgtttaagcgcgatgctcagggtcaacaaatgatattcccgtactatggtcacgaagaggagcttaggaagcacccgattaagcggtctacggttggatgggctacttcctctcttctcccggggacgagcgggggcagacaaaggcgggagctcgacccaatggacattagaggatcaatagtgtacctggagattgacaatcggcaatgcgtccaatctagcagtcaatgctttcagtctgcaaccgatgtcgcagcctttctgggcgccctggccagcctcgggtcccttaatatcccgtataagattgaggcggttaaatctgaacctgtcgaacctcccctcccctctcaattgcacctcatgtacgttgctgctgctgcctttgttctgctcttcttcgtaggatgcggggttcttctctccagaaaacgacggcgcggatttggtgatgtcggtgccctcgaaagtcttagaggcaacgctgatctggcgtatattttgagcatggaaccatgtggacattgtcttatcatcaataacgtaaatttttgtagggaaagtggccttaggactaggacaggttccaacatcgactgcgaaaaactgcggaggagattctcttccctccattttatggtggaagtcaaaggcgatctcaccgcaaagaagatggtactcgcattgctcgaacttgcccaacaggaccatggagctttggactgctgtgttgtagttatactgtcacatggttgccaggcaagccacctccagtttcctggggcggtatatgggaccgatggttgccccgtttccgtagagaagatagtaaatatcttcaacggaacgagctgtccgtccctcgggggcaagccaaagctttttttcattcaagcctgtggcggcgagcaaaaagaccacggattcgaggtggcatcaacgtcccctgaagatgagagtccgggcagcaatcccgaaccggatgctaccccattccaagaaggtcttaggacattcgaccaactggacgctatatctagtttgccaactccttcagatatctttgtaagttactctacgtttcctggcttcgtaagttggagagatcctaaatctggaagttggtatgtggaaacgttggatgacatttttgaacaatgggctcatagtgaagaccttcagtcactcctgcttcgcgtggcaaatgcagtgtcagttaagggtatctacaaacagatgccgtgcatcgtatccatgcttcggaaaaagctgttctttaaaacaagc

SEQ ID NO: 347 - DNA, 1494 bp, synthetic construct
  source 1..1494: mol_type = other DNA
ggtggaggcggttccacaacgaccttatctccatccggttccgcagtcattagcaccacgaccatagctaccacgccaagtaagccaacttgcgatgagaaatacgctaacatcaccgtggattatctgtataataaagaaacgaagttgttcacagctaagctcaatgtaaacgagaacgtggagtgcgggaataatacttgcactaacaacgaagtccataatctcactgagtgcaagaatgcttctgtgtccatttctcataactcttgcactgccccagataagacactgatactggatgtgccaccaggcgtggaaaagtttcagcttcacgactgtacccaggtcgagaaagctgacacgactatctgtctgaaatggaagaacatagaaacgtttacatgcgatacacagaacattacctatcgattccaatgcgggaacatgattttcgacaataaggaaattaaactggagaatctggagcctgaacatgagtacaaatgtgactccgaaatcctgtacaataatcacaagttcacaaatgcgagcaaaattatcaagactgacttcggatcaccaggcgaaccccagattattttctgtcgcagtgaggctgcacatcagggagtcataacatggaacccgccacagagaagtttccataactttacactctgttacataaaggagactgagaaagattgccttaacttagataagaacctgatcaagtacgaccttcagaacttgaaaccgtataccaagtatgtcctttccctgcacgcctacataatcgccaaagtgcagcgaaatggatctgctgccatgtgccatttcactacgaaatctgcaccaccttcccaggtgtggaacatgacagtgtctatgaccagtgacaattcaatgcacgttaaatgtagaccaccgagggatagaaatggtccacacgagaggtaccatttagaggtagaggcaggaaataccttagtgagaaatgagagccacaagaactgcgacttcagagtgaaagacttacagtactccactgattatacatttaaagcttatttccataatggagattaccccggcgaaccttttatactccaccactctactagctacaactccaaaatatacatttgggctccattggcgggcacgtgcggagttctgcttctcagtttggttattacctgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactg

SEQ ID NO: 348 - DNA, 2013 bp, synthetic construct
  source 1..2013: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattaccggaggtggtgggtcaggtggaggcggaagtatggtacgatggttccatcgcgacttgtccggactggacgctgagaccctcctgaaagggcggggtgtgcacggcagtttccttgcacgaccctccagaaagaatcaaggcgactttagtctttcagtaagggttggtgatcaggtcacacacattagaatccaaaattcaggtgacttctatgatttgtatggcggtgagaaatttgcaacgctcaccgaacttgttgaatattacacccagcaacagggagtacttcaggaccgcgacggtacaataattcaccttaagtaccccttgaattgttcagatcctacgtccgagagatggtatcacggacacatgagtggaggacaagctgaaaccctcctccaagcaaagggggaaccctggacttttttggtcagagagagtcttagccagccgggtgattttgttcttagtgtgctgtccgatcaaccgaaagcggggcctggttcacctttgcgcgtgacccatattaaagttatgtgtgaaggaggtcgatataccgttgggggccttgaaacattcgatagtctgacggatttggtcgaacacttcaaaaaaacagggattgaagaggcctccggcgcttttgtctatcttagacaaccctattatgcgacgagggtgaacgctgcggatatcgagaatagagttctggagctgaataaaaagcaagaatcagaggacaccgccaaagctggtttctgggaggagttcgagagtttgcagaagcaggaagttaaaaacctccaccagaggttggaagggcagagaccggaaaacaagggtaagaaccgctataaaaatatcttgcccttcgatcactctcgggtcatactgcagggtagagacagtaacattccaggcagtgattacatcaacgctaactatataaaaaatcagcttctgggcccagatgaaaatgcaaagacctatattgcgagtcagggctgtctggaggccacggttaatgacttctggcagatggcatggcaagaaaacagtagggtaatcgtcatgacaactagagaagttgagaaaggacggaacaagtgtgttccttactggcccgaggtaggcatgcagcgggcgtacgggccctacagtgttaccaactgcggcgaacatgataccacagagtataaattgcgaacactccaagtgtcaccactggataacggcgaccttatcagggagatctggcactaccaatacctgtcctggcctgatcacggcgtaccatccgaaccggggggagtgcttagctttctggatcagataaaccaaagacaggaatctctgccccatgcaggtccgatcatcgtccattgttccgcgggtataggtcggaccggaacgattatcgtaattgacatgttgatggaaaacatctctacgaaagggctcgattgcgatatagacatccagaagaccatacaaatggtacgggcacaaagatcaggcatggtccagaccgaagcccaatacaaattcatctacgtcgctattgcccagttcattgaaacaacgaaaaagaagttggaggttctccaatcccagaagggacaagagtctgagtacggtaacataacttatccgcctgccatgaaaaacgctcatgcaaaggcgagccggactagtagtaagcacaaagaagacgtttacgagaatctgcataccaaaaataagcgggaggaaaaagtaaaaaaacaacgatcagctgataaagagaaatctaaaggctcattgaagagaaaa

SEQ ID NO: 349 - DNA, 1578 bp, synthetic construct
  source 1..1578: mol_type = other DNA
actaccaccccagcacccagaccgccaactccggctcctacgatagcgtcccaacctctctctcttaggccagaggcatgccgcccggcagctgggggtgcggttcatactcggggacttgatttcgcttgtgacatatacatttgggctccattggcgggcacgtgcggagttctgcttctcagtttggttattaccggaggtggtgggtcaggtggaggcggaagtatgtccgccatacaggcagcatggcccagcggaacggaatgtatcgctaaatacaactttcacggcaccgcggagcaagatcttccgttctgtaaaggtgacgttcttaccatagtggcggttactaaggacccaaattggtataaagctaagaacaaggtcgggcgcgagggcataatcccagcaaactatgtccagaagagggaaggcgtgaaagcgggaaccaaattgagcctcatgccgtggttccacggcaagattacacgcgaacaagcggagcgactgctttatccaccagagacgggtcttttcctggtccgggagtccacgaattatccgggggactatacactttgtgtaagctgtgacggcaaggtcgaacactacaggatcatgtaccacgctagtaaacttagcatagatgaggaggtatattttgagaatctcatgcagcttgtagaacattacacaagtgacgcggatggattgtgtactcgattgattaagccaaaggttatggaaggtactgtcgcagcccaagacgagttttaccgatccggctgggctctgaacatgaaagagctcaaacttctccagaccatcggtaagggtgagtttggcgacgtaatgttgggtgattaccgcggaaataaggtggctgtaaaatgtatcaagaacgatgcaacagctcaagccttcctggctgaggcgtcagtgatgacacaacttagacactcaaacttggtccaattgctcggagttatcgtcgaagagaaaggcggcctctatattgttactgagtatatggctaaaggctcccttgtggactatcttcgatctcgaggcaggtctgttctcggtggggattgcttgttgaaattcagtcttgatgtttgcgaagctatggaatatctggaaggcaacaattttgtccatagggacctggccgcccggaatgtgttggtttcagaagataacgtggccaaggtgtccgactttggtctgacaaaagaggctagctccactcaggacactgggaagttgccggtaaaatggacggcccctgaagcattgagagagaaaaaattctctactaagtccgacgtgtggtcttttggcattcttctttgggaaatctactcattcggaagggtcccgtaccctcgcattccgcttaaggacgttgtccctcgggtcgaaaagggctacaagatggacgctcctgacggatgcccgcctgcggtctacgaggtaatgaaaaattgctggcatcttgatgctgcaatgcgcccaagtttcttgcaattgcgagagcaactggaacacataaagacccacgagctccatctc

SEQ ID NO: 350 - skipped

SEQ ID NO: 351 - DNA, 1017 bp, synthetic construct
  source 1..1017: mol_type = other DNA
accacgacaccggctcctcggccaccgacaccggcaccaacaattgcttcccagccactcagtctgcggccggaggcatgtcggcctgcggctggcggagctgttcacacgaggggcctcgattttgcatgcgatttctgggttcttgtagtagttggcggagtccttgcttgctacagtttgcttgtgacggtagcattcatcatattctgggtaggcggcgggggatctggaggtggtggcagcggtgggggcggtagtaatacgtgtaagctccttgtggtagcagaccataggttctacagatatatgggacgaggagaggaaagcactacgacaaactatttgatcgaacttatagatagagtcgacgacatctaccgaaacacgtcatgggacaatgcaggcttcaagggatacggaatacagatagagcagatacggattctgaaaagtccgcaggaagtgaaaccgggggagaaacattataatatggcgaaatcatatccaaacgaggagaaagatgcgtgggatgtaaaaatgctcttggaacagtttagttttgacattgctgaggaagccagcaaagtgtgcctcgctcacctgtttacttatcaagattttgatatgggcaccctgggtcttgcatatgtagggtctcccagagcaaactcccacgggggcgtatgtccaaaagcttactacagcccagtaggaaaaaagaatatatacctgaacagcgggctgactagtaccaaaaattacggaaaaactattctcacgaaggaggccgacttggtaactactcatgaactggggcataacttcggcgccgagcacgacccggacggacttgcagagtgcgccccgaacgaggaccaaggtggcaaatatgtcatgtacccaatcgccgtttcaggggatcatgagaacaataagatgttcagcaactgttccaaacaatccatttataaaacgatagaaagcaaagcccaggaatgttttcaggaaagatca

SEQ ID NO: 352 - protein, 528 aa, synthetic construct
VLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHAVSLSKMLKKRSPLTTGVYVKMPPTEPECEKQFQPYFIPINPARVTLTCLAVWGLCLLFALPDFIFLSAHHDERLNATHCQYNFPQVGRTALRVLQLVAGFLLPLLVMAYCYAHILAVLLVLTRKKKALRIHSVEGDLRRKSAGQEEWSPSAPSPPGSCVQAEAAPAGLCGEQRGEDCAELHDYFNVLSYRSLGNCSFFTETGQRRLRAMRLVVVVVVAFALCWTPYHLVVLVDILMDLGALARNCGRESRVDVAKSVTSGLGYMHCCLNPLLYAFVCSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 353 - DNA, 1584 bp, synthetic construct
  source 1..1584: mol_type = other DNA
gtccttgaggtgtccgaccaccaggtcctgaacgatgctgaggtggcagcgctgttggagaacttcagttcatcctacgattacggagagaacgagtctgatagttgttgtaccagtcccccctgcccccaggatttttcccttaatttcgacagggctttcctccccgcactgtattcccttctgtttcttttagggctgttaggcaatggcgccgtggcggctgtccttctgagtcggcgcacagccctctccagtacggatacctttctcctgcacctggcggtggccgacacgctgttggtcctgactcttccactctgggccgttgatgccgcagtccaatgggtttttggctccggtctgtgcaaggtagcgggagctctgttcaatatcaacttctacgctggagctctgctgctggcctgcatcagcttcgatagatatctgaacatcgtccatgcagtctcactctccaaaatgctgaaaaagcgctctccccttaccaccggcgtctatgtcaaaatgccacctactgaacctgaatgcgagaagcagttccaaccttattttatccccattaatccagccagagtaacactgacatgtctcgctgtctggggtctgtgcctgcttttcgccctgcctgacttcattttcctgtctgctcaccacgacgagcgtctgaatgccacccactgccagtataatttccctcaagtaggtcggaccgcccttcgagttttgcagctggtggccggtttcttattgcccttactggtgatggcttactgctacgcacacatcttggctgtgcttctcgttttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggtcagaggaggctgagggccatgagactcgttgtcgtcgtggttgttgctttcgccttatgctggaccccataccatttagttgtgctggtggatatcctgatggacctgggcgccctcgcacggaactgtggcagggaatcaagagtggacgtcgctaaatccgtgacctccggactgggttatatgcactgttgcctcaaccctctgctctacgcctttgtgtgtagtcgcgctgcaagaggtaccattggtgcgaggaggaccggacaacctttgaaggaggacccaagtgcggtacctgttttttccgtcgactacggcgagctggatttccagtggcgagaaaagacaccagaaccaccggttccttgtgtacctgagcagacggagtatgcgacaatcgtcttcccgagcggaatgggcacgagttcccccgccagaagggggtctgccgacggacctaggagcgcacaaccacttagacccgaagacggccattgttcctggccactg

SEQ ID NO: 354 - protein, 123 aa, synthetic construct
EVQLLESGGGLVQPGGSLRLSCAASGLTFSSYAMGWFRQAPGKEREVVSVISGTGRSTYYADSVKGRFTISRDNSKNTLYLQMNSLKPEDTAVYYCAAGISQYYGDVLLFDYWGQGTQVTVSS

SEQ ID NO: 355 - protein, 121 aa, synthetic construct
EVQLLESGGGLVQPGGSLRLSCAASGFSFSSYAMGWYRQAPGKEREGVSAISTTGGSSYYADSVKGRFTISRDNSKNTLYLQMNSLKPEDTAVYYCAQVLDVADRSEYPLWGQGTQVTVSS

SEQ ID NO: 356 - protein, 120 aa, synthetic construct
EVQLLESGGGLVQPGGSLRLSCAASGYTFSNYAMGWFRQAPGKEREFVSVISYSGGSTYYADSVKGRFTISRDNSKNTLYLQMNSLKPEDTAVYYCARRVAYQFRVYEYWGQGTQVTVSS

SEQ ID NO: 357 - protein, 120 aa, synthetic construct
EVQLLESGGGLVQPGGSLRLSCAASGFTFSSYAMGWYRQAPGKEREGVSAISGSGRSTYYADSVKGRFTISRDNSKNTLYLQMNSLKPEDTAVYYCAQVLSLSDEEGPYWGQGTQVTVSS

SEQ ID NO: 358 - protein, 120 aa, synthetic construct
EVQLLESGGGLVQPGGSLRLSCAASGYTFSSRAMGWYRQAPGKEREFVSVITGTGRTYYADSVKGRFTISRDNSKNTLYLQMNSLKPEDTAVYYCARIVLSSYSGFADYWGQGTQVTVSS

SEQ ID NO: 359 - protein, 123 aa, synthetic construct
EVQLLESGGGLVQPGGSLRLSCAASGYTFDNYGMGWFRQAPGKERELVSAISGSGSGTYYADSVKGRFTISRDNSKNTLYLQMNSLKPEDTAVYYCAADVAYLAVGWTGYEYWGQGTQVTVSS

SEQ ID NO: 360 - protein, 122 aa, synthetic construct
EVQLLESGGGLVQPGGSLRLSCAASGSTLDYYAMGWFRQAPGKEREFVSSISGTGGRTYYADSVKGRFTISRDNSKNTLYLQMNSLKPEDTAVYYCAADLGLYIVWELADYWGQGTQVTVSS

SEQ ID NO: 361 - protein, 119 aa, synthetic construct
EVQLLESGGGLVQPGGSLRLSCAASGLIFYIDAMGWFRQAPGKERELVSAISGSGGSIYYADSVKGRFTISRDNSKNTLYLQMNSLKPEDTAVYYCAADYYLGSEFDYWGQGTQVTVSS

SEQ ID NO: 362 - protein, 128 aa, synthetic construct
EVQLLESGGGLVQPGGSLRLSCAASGYTFSDYYMGWYRQAPGKERELVSAISGSGSETNYADSVKGRFTISRDNSKNTLYLQMNSLKPEDTAVYYCAVDVLYWYAADYWGQGTLTEYWGQGTQVTVSS

SEQ ID NO: 363 - protein, 117 aa, synthetic construct
EVQLLESGGGLVQPGGSLRLSCAASGSIFYIYAIGWYRQAPGKERELVSVISGSGGSTYYADSVKGRFTISRDNSKNTLYLQMNSLKPEDTAVYYCAVDAYTDFDYWGQGTQVTVSS

SEQ ID NO: 364 - DNA, 369 bp, synthetic construct
  source 1..369: mol_type = other DNA
gaggtacaacttttggagtcaggcggtggactggtacaaccgggtggttcattgcgtttgagctgcgctgcctctggtttgactttcagctcttacgctatgggttggttccgccaagcgccgggcaaagaacgcgaggtcgtatccgttataagcggtacggggagaagtacttactacgcggactcggtaaaaggccgttttacgatcagtcgtgataattccaagaataccttgtacctgcaaatgaatagccttaagcccgaagacacagcggtgtattattgtgccgccggcatatcccagtactacggcgatgtgctgttatttgactattggggccagggtacccaggtgacggtctcgagc

SEQ ID NO: 365 - DNA, 363 bp, synthetic construct
  source 1..363: mol_type = other DNA
gaggtacaacttttggagtcaggcggtggactggtacaaccgggtggttcattgcgtttgagctgcgctgcctctggtttttcattttcctcttacgctatgggctggtatcgccaagcgccgggcaaagaacgcgagggcgtctccgcaatatccacgaccgggggcagcagctattacgcggactcggtaaaaggccgttttacgatcagtcgtgataattccaagaataccttgtacctgcaaatgaatagccttaagcccgaagacacagcggtgtattattgtgcccaggttttagatgtagctgatcggtcagagtacccgttgtggggccagggtacccaggtgacggtctcgagc

SEQ ID NO: 366 - DNA, 360 bp, synthetic construct
  source 1..360: mol_type = other DNA
gaggtacaacttttggagtcaggcggtggactggtacaaccgggtggttcattgcgtttgagctgcgctgcctctggttatacttttagcaactacgctatgggatggttccgccaagcgccgggcaaagaacgcgagtttgtatccgtaatctcatacagcgggggatctacctactacgcggactcggtaaaaggccgttttacgatcagtcgtgataattccaagaataccttgtacctgcaaatgaatagccttaagcccgaagacacagcggtgtattattgtgccagaagagttgcttatcaatttcgggtatacgaatactggggccagggtacccaggtgacggtctcgagc

SEQ ID NO: 367 - DNA, 360 bp, synthetic construct
  source 1..360: mol_type = other DNA
gaggtacaacttttggagtcaggcggtggactggtacaaccgggtggttcattgcgtttgagctgcgctgcctctggttttacattttccagttacgcaatgggatggtatcgccaagcgccgggcaaagaacgcgagggtgtttccgcaatatcaggtagtggacgctccacctattacgcggactcggtaaaaggccgttttacgatcagtcgtgataattccaagaataccttgtacctgcaaatgaatagccttaagcccgaagacacagcggtgtattattgtgcccaagtgctttccctttccgatgaggaaggcccttattggggccagggtacccaggtgacggtctcgagc

SEQ ID NO: 368 - DNA, 360 bp, synthetic construct
  source 1..360: mol_type = other DNA
gaggtacaacttttggagtcaggcggtggactggtacaaccgggtggttcattgcgtttgagctgcgctgcctctggttatactttcagcagtcgggctatgggctggtatcgccaagcgccgggcaaagaacgcgagtttgtcagcgttataaccggtaccggccggacgtactacgcggactcggtaaaaggccgttttacgatcagtcgtgataattccaagaataccttgtacctgcaaatgaatagccttaagcccgaagacacagcggtgtattattgtgcccgaattgtgctgtcctcctattccggcttcgccgattattggggccagggtacccaggtgacggtctcgagc

SEQ ID NO: 369 - DNA, 369 bp, synthetic construct
  source 1..369: mol_type = other DNA
gaggtacaacttttggagtcaggcggtggactggtacaaccgggtggttcattgcgtttgagctgcgctgcctctggttacacttttgacaactatgggatgggctggttccgccaagcgccgggcaaagaacgcgagctggtatcagcaattagcggatcagggagtggtacgtactacgcggactcggtaaaaggccgttttacgatcagtcgtgataattccaagaataccttgtacctgcaaatgaatagccttaagcccgaagacacagcggtgtattattgtgccgcagatgtggcttatctggcagtcggctggacgggctatgaatactggggccagggtacccaggtgacggtctcgagc

SEQ ID NO: 370 - DNA, 366 bp, synthetic construct
  source 1..366: mol_type = other DNA
gaggtacaacttttggagtcaggcggtggactggtacaaccgggtggttcattgcgtttgagctgcgctgcctctggttccacgcttgactactatgcgatgggatggttccgccaagcgccgggcaaagaacgcgagtttgtttcctccatctctgggaccggtggtcgtacttattacgcggactcggtaaaaggccgttttacgatcagtcgtgataattccaagaataccttgtacctgcaaatgaatagccttaagcccgaagacacagcggtgtattattgtgccgctgacttgggcctgtacatagtctgggagctggccgactactggggccagggtacccaggtgacggtctcgagc

SEQ ID NO: 371 - DNA, 357 bp, synthetic construct
  source 1..357: mol_type = other DNA
gaggtacaacttttggagtcaggcggtggactggtacaaccgggtggttcattgcgtttgagctgcgctgcctctggtctgatattctatatcgacgcaatgggttggtttcgccaagcgccgggcaaagaacgcgagttggtttctgcgatttccggctctggaggaagtatctattacgcggactcggtaaaaggccgttttacgatcagtcgtgataattccaagaataccttgtacctgcaaatgaatagccttaagcccgaagacacagcggtgtattattgtgccgcggactattacttggggagtgagtttgactactggggccagggtacccaggtgacggtctcgagc

SEQ ID NO: 372 - DNA, 384 bp, synthetic construct
  source 1..384: mol_type = other DNA
gaggtacaacttttggagtcaggcggtggactggtacaaccgggtggttcattgcgtttgagctgcgctgcctctggttatacgtttagtgattactatatgggttggtatcgccaagcgccgggcaaagaacgcgagttagtgtcagctatttctgggtcaggttccgagaccaattacgcggactcggtaaaaggccgttttacgatcagtcgtgataattccaagaataccttgtacctgcaaatgaatagccttaagcccgaagacacagcggtgtattattgtgccgtcgatgttctgtactggtatgccgccgactactggggccagggtaccctgacggaatattggggccagggtacccaggtgacggtctcgagc

SEQ ID NO: 373 - DNA, 351 bp, synthetic construct
  source 1..351: mol_type = other DNA
gaggtacaacttttggagtcaggcggtggactggtacaaccgggtggttcattgcgtttgagctgcgctgcctctggtagtattttttatatttatgctattggctggtatcgccaagcgccgggcaaagaacgcgagctggtttctgttatttccgggtcgggtggctcgacctattacgcggactcggtaaaaggccgttttacgatcagtcgtgataattccaagaataccttgtacctgcaaatgaatagccttaagcccgaagacacagcggtgtattattgtgccgttgatgcttacaccgactttgattactggggccagggtacccaggtgacggtctcgagc

SEQ ID NO: 374 - DNA, 63 bp, synthetic construct
  source 1..63: mol_type = other DNA
atatacatttgggctccattggcgggcacgtgcggagttctgcttctcagtttggttattacc

SEQ ID NO: 375 - DNA, 63 bp, synthetic construct
  source 1..63: mol_type = other DNA
gtgggagttgttgggggacttcttggatctcttgtccttcttgtatgggtgttggcagtcata

SEQ ID NO: 376 - DNA, 81 bp, synthetic construct
  source 1..81: mol_type = other DNA
ttttgggtgctggtggtggttggtggagtcctggcttgctattccttgctagtaacagtggccttcatcatcttttgggtc

SEQ ID NO: 377 - DNA, 978 bp, synthetic construct
  source 1..978: mol_type = other DNA
attttggattatagtttcaccggaggcgccggacgcgacatacctcccccgcaaatcgaggaagcgtgcgaactcccagagtgtcaagtagacgccggcaacaaggtgtgcaacctgcaatgcaacaaccatgcctgcggatgggatggaggggactgtagtttgaatttcaacgacccttggaagaactgcacgcagtctcttcaatgctggaaatatttcagcgatggacattgtgacagccagtgtaacagcgccggatgcttgtttgatggctttgattgtcaattgaccgagggtcagtgcaacccgctctatgaccaatattgtaaggatcacttttccgacgggcactgcgatcagggctgcaatagcgcggaatgtgaatgggacggattggattgtgcagaacatgtgcccgaacgcctcgcggcaggaactctggtcctcgtagtcttgttgccgcccgaccaattgcggaacaacagtttccactttctgcgggaattgtcacatgtgctgcatacaaatgtcgtgtttaagcgcgatgctcagggtcaacaaatgatattcccgtactatggtcacgaagaggagcttaggaagcacccgattaagcggtctacggttggatgggctacttcctctcttctcccggggacgagcgggggcagacaaaggcgggagctcgacccaatggacattagaggatcaatagtgtacctggagattgacaatcggcaatgcgtccaatctagcagtcaatgctttcagtctgcaaccgatgtcgcagcctttctgggcgccctggccagcctcgggtcccttaatatcccgtataagattgaggcggttaaatctgaacctgtcgaacctcccctcccctctcaattgcacctcatgtacgttgctgctgctgcctttgttctgctcttcttcgtaggatgcggggttcttctctccagaaaacgacggcgc

SEQ ID NO: 378 - DNA, 63 bp, synthetic construct
  source 1..63: mol_type = other DNA
atggccagatccccggcacaactgctcggactcctcctgttgtggttgagcggggcccgctgt

(19) United States
(12) Patent Application Publication
(10) Pub. No.: US 2026/0199463 A1
(43) Pub. Date: Jul. 16, 2026

(54) GENETICALLY ENGINEERED CELLS HAVING ANTI-NECTIN4 CHIMERIC ANTIGEN RECEPTORS, AND USES THEREOF

(71) Applicant: Century Therapeutics, Inc., Philadelphia, PA (US)

(72) Inventors: Luis BORGES, Philadelphia, PA (US); Jill Marinari CARTON, Philadelphia, PA (US); Michael Francis NASO, Philadelphia, PA (US); Mark WALLET, Philadelphia, PA (US); John WHEELER, Philadelphia, PA (US); Matthew S. HALL, Philadelphia, PA (US)

(21) Appl. No.: 19/128,936

(22) PCT Filed: Nov. 10, 2023
(86) PCT No.: PCT/US2023/079401

§ 371 Date: May 9, 2025

Related U.S. Application Data
(60) Provisional application No. 63/383,089, filed on Nov. 10, 2022.

(51) Int. Cl. A61K 40/11; A61K 35/17; A61K 35/545; A61K 40/15; A61K 40/31; A61K 40/42; A61K 45/06; A61P 35/00; C07K 14/705; C12N 5/074; C12N 5/0783; C12N 5/0789; C12N 5/10

(52) U.S. Cl. CPC A61K 40/11; A61K 35/17; A61K 35/545; A61K 40/15; A61K 40/31; A61K 40/4254; A61K 45/06; A61P 35/00; C07K 14/70503; C12N 5/0636; C12N 5/0646; C12N 5/0647; C12N 5/0696; C12N 5/10; C07K 2317/53; C07K 2317/56; C07K 2319/02; C07K 2319/03; C12N 2510/00

(57) ABSTRACT

Provided are genetically engineered induced pluripotent stem cells (iPSCs) and derivative cells thereof expressing an anti-Nectin4 chimeric antigen receptor (CAR) and, optionally, an inhibitory CAR, and methods of using the same. Also provided are compositions, polypeptides, vectors, and methods of manufacturing.

### CROSS-REFERENCE TO RELATED APPLICATIONS

This application claims the benefit of U.S. Provisional Patent Application No. 63/383,989 filed Nov. 10, 2022, which is incorporated by reference herein in its entirety.

### TECHNICAL FIELD

This application provides genetically engineered induced pluripotent stem cells (iPSCs) and derivative cells thereof. Also provided are uses of the iPSCs or derivative cells thereof to express a chimeric antigen receptor for allogenic cell therapy. Also provided are related vectors, polynucleotides, and pharmaceutical compositions.

### REFERENCE TO SEQUENCE LISTING SUBMITTED ELECTRONICALLY

This application contains a sequence listing, which is submitted electronically via EFS-Web as an XML formatted sequence listing with a file name “SequenceListing_ST26” having a file size of 536 kilobytes, and a creation date of Nov. 9, 2023. The sequence listing submitted via EFS-Web is part of the specification and is herein incorporated by reference in its entirety.

### BACKGROUND

Chimeric antigen receptors (CARs) have shown remarkable activity in cancer treatment by enhancing anti-tumor activity of immune effector cells. CAR-T cells are engineered to target antigens expressed on cancer cells. However, some of these antigens may also be expressed at low levels on normal healthy tissues. This can lead to on-target/off-tumor toxicity if the CAR-T cells attack those healthy tissues. The most well-known example is CAR-T cells targeting CD19 to treat B-cell malignancies. CD19 is also expressed on normal B cells, so patients can experience B cell adverse effects from treatment (e.g., aplasia or hypogammaglobulinemia). Other tumor antigens like ERBB2 (HER2) and EGFR have some expression on epithelial cells of the lung, liver and skin, resulting in toxicity to those tissues from treatment. Careful antigen selection and engineering of the CAR construct is needed to maximize specificity.

To address these challenges, embodiments of the present disclosure are designed to increase depth and durability of response by targeting Nectin4, a tumor associated marker for many tumors including lung, breast, colon, bladder, renal, head and neck, esophageal, and ovarian cancers. The present disclosure also provides cells that are genetically engineered to express an additional antigen binder targeting a healthy cell antigen (e.g., DSG1) to reduce off-target binding/improve tumor target specificity, either by genetically engineering the cell to express an additional inhibitory CAR in combination with an anti-Nectin4 CAR, or by engineering the cell to express a dual-targeting CAR targeting Nectin4 and a healthy cell antigen.

### BRIEF SUMMARY

In one general aspect, the present disclosure provides an induced pluripotent stem cell (iPSC) or a derivative cell thereof comprising: one or more exogenous polynucleotides encoding a chimeric antigen receptor (CAR) comprising an antigen binding domain targeting a Nectin4 antigen; and at least one of: (i) a deletion or reduced expression of one or more of B2M, TAP 1, TAP 2, Tapasin, RFXANK, CIITA, RFX5, RFXAP genes; (ii) an exogenous polynucleotide encoding a human leukocyte antigen E (HLA-E) and/or human leukocyte antigen G (HLA-G); (iii) an exogenous polynucleotide encoding a natural killer (NK) cell receptor immunoglobulin gamma Fc region receptor III (FcγRIII), cluster of differentiation 16 (CD16) and/or an NKG2D protein; (iv) a deletion or reduced expression of one or more of NKG2A or CD70, CD38, and CD33 genes; (v) an exogeneous polynucleotide encoding a cytokine; (vi) an exogenous polynucleotide encoding a safety switch; (vii) an exogeneous polynucleotide encoding a PSMA cell tracer; and (viii) an exogeneous polynucleotide encoding a membrane bound IL-12 polypeptide. In certain embodiments, the CAR can be a dual-targeting CAR comprising an additional antigen-binding domain that specifically binds to an antigen selected from the group consisting of CD70, Folate Receptor alpha, FSHR, mesothelin, and SLITRK6. In certain embodiments, the cell can comprise one or more exogenous polynucleotides encoding an additional CAR comprising an antigen-binding domain that specifically binds to an antigen selected from the group consisting of CD70, Folate Receptor alpha, FSHR, mesothelin, and SLITRK6. In certain embodiments, the CAR can comprise an anti-Nectin4 VHH domain. In certain embodiments, the cytokine can comprise an IL-15. In certain embodiments, the iPSC or derivative cell thereof can further comprise an inactivated cell surface receptor that can comprise a monoclonal antibody-specific epitope, wherein the inactivated cell surface receptor and the IL-15 can be operably linked by an autoprotease peptide. In certain embodiments, the IL-15 can comprise an IL-15 and an IL-15 receptor alpha (IL-15Rα) fusion polypeptide. In certain embodiments, the IL-15 can comprise amino acids having at least 90%, 91%, 92%, 93%, 94%, 95%, 96%, 97%, 98%, 99% or 100% sequence identity to SEQ ID NO: 72. In certain embodiments, the iPSC or derivative cell thereof can comprise the deletion or reduced expression of one or more of B2M, TAP 1, TAP 2, Tapasin, RFXANK, CIITA, RFX5 and RFXAP genes. In certain embodiments, the iPSC or derivative cell thereof can comprise an exogenous polynucleotide encoding a human leukocyte antigen E (HLA-E) and/or human leukocyte antigen G (HLA-G). In certain embodiments, the CD16 can be a CD16 variant protein. In certain embodiments, the CD16 variant protein can be a high affinity CD16 variant. In certain embodiments, the CD16 variant protein can be a non-cleavable CD16 variant. In certain embodiments, the CD16 variant protein can comprise wild-type CD16 having one or more amino acid substitutions selected from the group consisting of F158V, F176V, S197P, D205A, S219A, T220A. In certain embodiments, the CD16 variant protein can comprise amino acids having at least 90%, 91%, 92%, 93%, 94%, 95%, 96%, 97%, 98%, 99% or 100% sequence identity to any one of SEQ ID NOS:187 and 188. In certain embodiments, the iPSC or the derivative cell thereof can comprise an exogenous polynucleotide encoding the CD16 protein and the NKG2D protein, wherein the CD16 protein and the NKG2D protein can be operably linked by an autoprotease peptide. In certain embodiments, the NKG2D protein can be a wildtype NKG2D protein. In certain embodiments, the NKG2D protein can comprise amino acids having at least 90%, 91%, 92%, 93%, 94%, 95%, 96%, 97%, 98%, 99% or 100% sequence identity to SEQ ID NO: 190. In certain embodiments, the autoprotease peptide can be selected from the group consisting of a porcine teschovirus-1 2A (P2A) peptide, a foot-and-mouth disease virus 2A (F2A) peptide, an Equine Rhinitis A Virus (ERAV) 2A (E2A) peptide, a Thosea asigna virus 2A (T2A) peptide, a cytoplasmic polyhedrosis virus 2A (BmCPV2A) peptide, and a Flacherie Virus 2A (BmIFV2A) peptide. In certain embodiments, the autoprotease peptide can be a P2A peptide comprising amino acids having at least 90% sequence identity to SEQ ID NO: 192. In certain embodiments, the exogenous polynucleotide encoding the CD16 protein and the NKG2D protein can comprise polynucleotides having at least 90%, 91%, 92%, 93%, 94%, 95%, 96%, 97%, 98%, 99% or 100% sequence identity to SEQ ID NO: 193. In certain embodiments, one or more of the exogenous polynucleotides can be integrated at one or more loci on the chromosome of the cell selected from the group consisting of AAVS1, CLYBL, CCR5, ROSA26, collagen, HTRP, Hl 1, GAPDH, RUNX1, B2M, TAP1, TAP2, Tapasin, NLRC5, RFXANK, CIITA, RFX5, RFXAP, TCR a or b constant region, NKG2A, NKG2D, CD33, CD38, CD70, TRAC, CIS, CBL-B, SOCS2, PD1, CTLA4, LAG3, TIM3, and TIGIT genes, provided at least one of the exogenous polynucleotides can be integrated at a locus of a gene selected from the group consisting of B2M, TAP 1, TAP 2, Tapasin, RFXANK, CIITA, RFX5 and RFXAP genes to thereby result in a deletion or reduced expression of the gene. In certain embodiments, one or more of the exogenous polynucleotides can be integrated at the loci of the AAVS1 and B2M genes. In certain embodiments, the iPSC or the derivative cell thereof can comprise a deletion or reduced expression of one or more of B2M or CIITA genes. In certain embodiments, the iPSC or the derivative cell thereof can comprise the deletion or reduced expression of B2M and CIITA genes. In certain embodiments, the iPSC can be reprogrammed from whole peripheral blood mononuclear cells (PBMCs). In certain embodiments, the iPSC can be derived from a re-programmed T-cell. In certain embodiments, the CAR can comprise: (i) a signal peptide; (ii) an extracellular domain comprising a binding domain that specifically binds the Nectin4 antigen; (iii) a hinge region; (iv) a transmembrane domain; (v) an intracellular signaling domain; and (vi) a co-stimulatory domain. In certain embodiments, the extracellular domain can comprise a VHH single domain antibody that specifically binds the Nectin4 antigen. In certain embodiments, the extracellular domain can comprise amino acids having at least 90%, 91%, 92%, 93%, 94%, 95%, 96%, 97%, 98%, 99% or 100% sequence identity to one of SEQ ID NOs: 105-130. In certain embodiments, the extracellular domain can be encoded by a polynucleotide having at least 90%, 91%, 92%, 93%, 94%, 95%, 96%, 97%, 98%, 99% or 100% sequence identity to one of SEQ ID NOs: 131-156. In certain embodiments, the CAR can be encoded by a polynucleotide having at least 90%, 91%, 92%, 93%, 94%, 95%, 96%, 97%, 98%, 99% or 100% sequence identity to one of SEQ ID NOs: 171-184. In certain embodiments, the additional CAR can comprise: (i) a signal peptide; (ii) an additional extracellular domain comprising a binding domain that specifically binds to an antigen selected from the group consisting of CD70, Folate Receptor alpha, FSHR, mesothelin, and SLITRK6; (iii) a hinge region; (iv) a transmembrane domain; (v) an intracellular signaling domain; and (vi) a co-stimulatory domain. In certain embodiments, the additional extracellular domain can comprise a VHH or an scFv that specifically binds to an antigen selected from the group consisting of CD70, Folate Receptor alpha, FSHR, mesothelin, and SLITRK6. In certain embodiments, the signal peptide can comprise a GMCSFR signal peptide or a MARS signal peptide. In certain embodiments, the hinge region for each of the CAR and the additional CAR can be independently selected from the group consisting of a CD28 hinge region, an IgG4 hinge region, and a CD8 hinge region. In certain embodiments, the transmembrane domain for each of the CAR and the additional CAR can be independently selected from the group consisting of a CD28 transmembrane domain and a CD8 transmembrane domain. In certain embodiments, the intracellular signaling domain can comprise a CD3ζ intracellular domain. In certain embodiments, the co-stimulatory domain for each of the CAR and the additional CAR can be independently selected from the group consisting of a CD28 signaling domain, a 41BB signaling domain, and a DAP10 signaling domain. In certain embodiments, in the CAR: (i) the signal peptide can comprise amino acids having at least 90%, 91%, 92%, 93%, 94%, 95%, 96%, 97%, 98%, 99% or 100% sequence identity to SEQ ID NOs: 1, 97, or 98; (ii) the extracellular domain can comprise amino acids having at least 90%, 91%, 92%, 93%, 94%, 95%, 96%, 97%, 98%, 99% or 100% sequence identity to SEQ ID NOs: 105-130, or the extracellular domain can be encoded by a polynucleotide having at least 90%, 91%, 92%, 93%, 94%, 95%, 96%, 97%, 98%, 99% or 100% sequence identity to SEQ ID NOs: 131-156; (iii) the hinge region can comprise amino acids having at least 90%, 91%, 92%, 93%, 94%, 95%, 96%, 97%, 98%, 99% or 100% sequence identity to SEQ ID NO: 21 or 96; (iv) the transmembrane domain can comprise amino acids having at least 90%, 91%, 92%, 93%, 94%, 95%, 96%, 97%, 98%, 99% or 100% sequence identity to SEQ ID NO: 23 or 24; (v) the intracellular signaling domain can comprise amino acids having at least 90%, 91%, 92%, 93%, 94%, 95%, 96%, 97%, 98%, 99% or 100% sequence identity to SEQ ID NO: 6, or the intracellular signaling domain can be encoded by a polynucleotide having at least 90%, 91%, 92%, 93%, 94%, 95%, 96%, 97%, 98%, 99% or 100% sequence identity to SEQ ID NO: 101; and (vi) the co-stimulatory domain can comprise amino acids having at least 90%, 91%, 92%, 93%, 94%, 95%, 96%, 97%, 98%, 99% or 100% sequence identity to SEQ ID NO: 8 or 17. In certain embodiments, in the CAR: (i) the signal peptide can comprise amino acids having the sequence of SEQ ID NO: 1, 97, or 98; (ii) the extracellular domain can comprise amino acids having the sequence of one of SEQ ID NOs: 105-130; (iii) the hinge region can comprise amino acids having the sequence of SEQ ID NO: 21 or 96; (iv) the transmembrane domain can comprise amino acids having the sequence of SEQ ID NO: 23 or 24; (v) the intracellular signaling domain can comprise amino acids having the sequence of SEQ ID NO: 6, or the intracellular signaling domain can be encoded by the polynucleotide having the sequence of SEQ ID NO: 101; and (vi) the co-stimulatory domain can comprise amino acids having the sequence of SEQ ID NO: 8 or 17. In certain embodiments, the iPSC or the derivative cell can comprise an exogenous polynucleotide encoding a safety switch. In certain embodiments, the safety switch can comprise an exogenous polynucleotide encoding an inactivated cell surface receptor that can comprise a monoclonal antibody-specific epitope. In certain embodiments, the inactivated cell surface receptor can be selected from the group of monoclonal antibody specific epitopes selected from epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, avelumab, ofatumumab, panitumumab, and ustekinumab. In certain embodiments, the inactivated cell surface receptor can be a truncated epithelial growth factor (tEGFR) variant. In certain embodiments, the tEGFR variant consists of amino acids having at least 90%, 91%, 92%, 93%, 94%, 95%, 96%, 97%, 98%, 99% or 100% sequence identity to SEQ ID NO: 71. In certain embodiments, the safety switch can comprise an intracellular domain having a herpes simplex virus thymidine kinase (HSV-TK). In certain embodiments, the iPSC or the derivative cell can comprise the exogeneous polynucleotide encoding the PSMA cell tracer, wherein the PSMA cell tracer can comprise an extracellular domain comprising a PSMA extracellular domain or fragment thereof. In certain embodiments, the iPSC the derivative cell thereof can comprise a combined artificial cell death/reporter system polypeptide comprising an intracellular domain having a herpes simplex virus thymidine kinase (HSV-TK) and a linker, a transmembrane region, and an extracellular domain comprising the PSMA extracellular domain or fragment thereof. In certain embodiments, the HSV-TK can comprise amino acids having at least 90%, 91%, 92%, 93%, 94%, 95%, 96%, 97%, 98%, 99% or 100% sequence identity to SEQ ID NO: 229 or 230. In certain embodiments, the combined artificial cell death/reporter system polypeptide can comprise the HSV-TK fused to a truncated variant PSMA polypeptide via the linker. In certain embodiments, the truncated variant PSMA polypeptide can comprise amino acids having at least 90%, 91%, 92%, 93%, 94%, 95%, 96%, 97%, 98%, 99% or 100% sequence identity to SEQ ID NO: 231. In certain embodiments, the linker can comprise an autoprotease peptide sequence selected from the group consisting of P2A peptide sequence, T2A peptide sequence, E2A peptide sequence, and F2A peptide sequence. In certain embodiments, the artificial cell death/reporter system polypeptide can comprise amino acids having at least 90%, 91%, 92%, 93%, 94%, 95%, 96%, 97%, 98%, 99% or 100% sequence identity to SEQ ID NO: 232. In certain embodiments, the artificial cell death/reporter system polypeptide can comprise amino acids having at least 90%, 91%, 92%, 93%, 94%, 95%, 96%, 97%, 98%, 99% or 100% sequence identity to a sequence selected from the group consisting of SEQ ID NOS: 233-235. In certain embodiments, the artificial cell death/reporter system polypeptide can be encoded by a polynucleotide having at least 90%, 91%, 92%, 93%, 94%, 95%, 96%, 97%, 98%, 99% or 100% sequence identity to a sequence selected from the group consisting of SEQ ID NOS: 236-238. In certain embodiments, the HLA-E can comprise amino acids having at least 90%, 91%, 92%, 93%, 94%, 95%, 96%, 97%, 98%, 99% or 100% sequence identity to SEQ ID NO: 66 and/or the HLA-G can comprise amino acids having at least 90%, 91%, 92%, 93%, 94%, 95%, 96%, 97%, 98%, 99% or 100% sequence identity to SEQ ID NO: 69. In certain embodiments, (i) the one or more exogenous polynucleotides encoding a chimeric antigen receptor (CAR) comprising an antigen binding domain targeting a Nectin4 antigen can comprise nucleotides having at least 90%, 91%, 92%, 93%, 94%, 95%, 96%, 97%, 98%, 99% or 100% sequence identity to one or more selected from the group consisting of SEQ ID NOs: 171-184; (ii) the exogenous polynucleotide encoding a human leukocyte antigen E (HLA-E) and/or human leukocyte antigen G (HLA-G) can comprise nucleotides having at least 90%, 91%, 92%, 93%, 94%, 95%, 96%, 97%, 98%, 99% or 100% sequence identity to one or more of SEQ ID NOs: 67 and 70; (iii) the exogenous polynucleotide encoding an NK cell receptor immunoglobulin gamma Fc region receptor III (FcγRIII, cluster of differentiation 16 (CD16)) and/or an NKG2D protein can comprise nucleotides having at least 90%, 91%, 92%, 93%, 94%, 95%, 96%, 97%, 98%, 99% or 100% sequence identity to one or more of SEQ ID NOs: 185, 189, and 191; (iv) the exogeneous polynucleotide encoding a cytokine can comprise nucleotides having at least 90%, 91%, 92%, 93%, 94%, 95%, 96%, 97%, 98%, 99% or 100% sequence identity to SEQ ID NO: 239; (v) the exogenous polynucleotide encoding a safety switch can comprise nucleotides having at least 90%, 91%, 92%, 93%, 94%, 95%, 96%, 97%, 98%, 99% or 100% sequence identity to one or more of SEQ ID NO: 236-238; and/or (vi) the exogeneous polynucleotide encodes a PSMA cell tracer, and the PSMA cell tracer can comprise amino acids having at least 90%, 91%, 92%, 93%, 94%, 95%, 96%, 97%, 98%, 99% or 100% sequence identity to SEQ ID NO: 231. In certain embodiments, (i) the one or more exogenous polynucleotides encoding a chimeric antigen receptor (CAR) comprising an antigen binding domain targeting a Nectin4 antigen can comprise nucleotides having a sequence selected from the group consisting of SEQ ID NOs: 171-184; (ii) the exogenous polynucleotide encoding a human leukocyte antigen E (HLA-E) and/or human leukocyte antigen G (HLA-G) can comprise nucleotides having a sequence SEQ ID NO: 67 or 70; (iii) the exogenous polynucleotide encoding an NK cell receptor immunoglobulin gamma Fc region receptor III (FcγRIII, cluster of differentiation 16 (CD16)) and/or an NKG2D protein can comprise nucleotides having a sequence of SEQ ID NO: 185, 189, or 191; (iv) the exogeneous polynucleotide encoding the cytokine can comprise nucleotides having a sequence of SEQ ID NO: 239; and/or (v) the exogenous polynucleotide encoding the safety switch can comprise nucleotides having a sequence of one of SEQ ID NOs: 236-238. In certain embodiments, the exogenous polynucleotides can be integrated into a gene locus independently selected from the group consisting of an AAVS1 locus, a B2M locus, a CIITA locus, a CCR5 locus, a CD70 locus, a CLYBL locus, an NKG2A locus, an NKG2D locus, a CD33 locus, a CD38 locus, a TRAC locus, a TRBC1 locus, a ROSA26 locus, an HTRP locus, a GAPDH locus, a RUNX1 locus, a TAP1 locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus. In certain embodiments, (i) the one or more exogenous polynucleotides encoding a chimeric antigen receptor (CAR) comprising an antigen binding domain targeting a Nectin4 antigen can be integrated at a locus of the AAVS1 gene; (ii) the exogenous polynucleotide encoding a human leukocyte antigen E (HILA-E) and/or human leukocyte antigen G (HLA-G) can be integrated at a locus of the B2M gene; In certain embodiments, (iii) the exogenous polynucleotide encoding an NK cell receptor immunoglobulin gamma Fc region receptor III (FcγRIII, cluster of differentiation 16 (CD16)) and/or an NKG2D can be integrated at a locus of the CD70 gene; (iv) the exogeneous polynucleotide encoding the cytokine can be integrated at the locus of the NKG2A gene; (v) the exogenous polynucleotide encoding a safety switch can be integrated at the locus of the CLYBL gene; and (vi) there can be a deletion or reduced expression of the CIITA gene. In certain embodiments, the one or more exogenous polynucleotides further encode one or more inhibitory CARs (iCARs) comprising at least one antigen binding domain targeting an antigen independently selected from the group consisting of Adrenoceptor Beta 2 (ADRB2), Aquaporin 4 (AQP4), Claudin 10 (CLDN10B), Desmocollin (DSC) 1, DSC3, Desmoglein (DSG) 1, DSG3, Glycerophosphodiester Phosphodiesterase Domain Containing 2 (GDPD2), Hydroxycarboxylic Acid Receptor 3 (HCAR3), Lymphocyte Antigen 6 Family Member D (LY6D), and V-Set And Immunoglobulin Domain Containing 8 (VSIG8). In certain embodiments, the iCAR can comprise: (i) a signal peptide; (ii) an extracellular domain comprising an antigen binding domain that specifically binds at least one antigen selected from the group consisting of Adrenoceptor Beta 2 (ADRB2), Aquaporin 4 (AQP4), Claudin 10 (CLDN10B), Desmocollin (DSC) 1, DSC3, Desmoglein (DSG) 1, DSG3, Glycerophosphodiester Phosphodiesterase Domain Containing 2 (GDPD2), Hydroxycarboxylic Acid Receptor 3 (HCAR3), Lymphocyte Antigen 6 Family Member D (LY6D), V-Set And Immunoglobulin Domain Containing 8 (VSIG8); (iii) a hinge region; (iv) one or more transmembrane domains; (v) an intracellular signaling domain; and/or (vi) a co-stimulatory domain. In certain embodiments, the extracellular domain of the iCAR can comprise amino acids having at least 90%, 91%, 92%, 93%, 94%, 95%, 96%, 97%, 98%, 99% or 100% sequence identity to one or more of SEQ ID NOs: 354-363. In certain embodiments, the extracellular domain of the iCAR can be encoded by a polynucleotide having at least 90%, 91%, 92%, 93%, 94%, 95%, 96%, 97%, 98%, 99% or 100% sequence identity to one or more of SEQ ID NOs: 364-373. In certain embodiments, the signal peptide of the iCAR can comprise a CD8 signal peptide, a GMCSFR signal peptide, a MARS signal peptide, or an IgK signal peptide or variant thereof. In certain embodiments, the signal peptide of the iCAR can comprise amino acids having at least 90%, 91%, 92%, 93%, 94%, 95%, 96%, 97%, 98%, 99% or 100% sequence identity to one of SEQ ID NOs: 97 and 292. In certain embodiments, the signal peptide of the iCAR can be encoded by a polynucleotide having at least 90%, 91%, 92%, 93%, 94%, 95%, 96%, 97%, 98%, 99% or 100% sequence identity to one of SEQ ID NOs: 98, 327, and 378. In certain embodiments, the hinge region of the iCAR can be selected from the group consisting of a CD28 hinge region, a CD45 hinge region, a G4S-CD45 hinge region, a CD8 hinge region, and a CXC3R GPCR hinge region. In certain embodiments, the hinge region of the iCAR can comprise amino acids having at least 90%, 91%, 92%, 93%, 94%, 95%, 96%, 97%, 98%, 99% or 100% sequence identity to one of SEQ ID NOs: 21, 22, 288, 289, 319, and 321. In certain embodiments, the hinge region of the iCAR can be encoded by a polynucleotide having at least 90%, 91%, 92%, 93%, 94%, 95%, 96%, 97%, 98%, 99% or 100% sequence identity to one of SEQ ID NOs: 315-318, 320, and 322. In certain embodiments, the one or more transmembrane domains of the iCAR can be independently selected from the group consisting of a CD28 transmembrane domain, a CD8 transmembrane domain, a PD1 transmembrane domain, a SynNotch transmembrane domain, and a CXC3R GPCR. In certain embodiments, the transmembrane domain of the iCAR can comprise amino acids having at least 90%, 91%, 92%, 93%, 94%, 95%, 96%, 97%, 98%, 99% or 100% sequence identity to one of SEQ ID NOs: 23, 24, 290, 291, 323, and 325. In certain embodiments, the transmembrane domain of the iCAR can be encoded by a polynucleotide having at least 90%, 91%, 92%, 93%, 94%, 95%, 96%, 97%, 98%, 99% or 100% sequence identity to one of SEQ ID NOs: 324, 326, and 374-377. In certain embodiments, the intracellular signaling domain of the iCAR can comprise one or more of a PD1 intracellular domain, an LIRB1 intracellular domain, a TIGIT a CTLA4 intracellular domain, a CSK*(YSSV) intracellular domain, a KIR2DL1 intracellular domain, a DR1 intracellular domain, a Casp8 wt intracellular domain, a tCasp8 intracellular domain, a tCasp8-dimer intracellular domain, a tBid15 intracellular domain, a Casp9 wt intracellular domain, a tCasp9 intracellular domain, a tCasp9-dimer intracellular domain, a SHP1 intracellular domain, a (G4S)2-SHP1 intracellular domain, a CSK intracellular domain, a (G4S)2-CSK intracellular domain, an ADAM17 cleavage site, a CD28 intracellular domain, a CD3ζ intracellular domain, a G4S3 linker, an ADAM 17 protease domain, and a (G4S)3-ADAM 17 protease domain. In certain embodiments, the intracellular signaling domain of the iCAR can comprise amino acids having at least 90%, 91%, 92%, 93%, 94%, 95%, 96%, 97%, 98%, 99% or 100% sequence identity to one of SEQ ID NOs: 6, 8, and 267-287. In certain embodiments, the intracellular signaling domain of the iCAR can be encoded by a polynucleotide having at least 90%, 91%, 92%, 93%, 94%, 95%, 96%, 97%, 98%, 99% or 100% sequence identity to one of SEQ ID NOs: 266, and 293-314. In certain embodiments, the co-stimulatory domain of the iCAR can be selected from the group consisting of a CD28 signaling domain, a 41BB signaling domain, and a DAP10 signaling domain. In certain embodiments, in the iCAR: (i) the signal peptide can comprise amino acids having at least 90%, 91%, 92%, 93%, 94%, 95%, 96%, 97%, 98%, 99% or 100% sequence identity to SEQ ID NO: 97 or 292, or the signal peptide can be encoded by a polynucleotide having at least 90%, 91%, 92%, 93%, 94%, 95%, 96%, 97%, 98%, 99% or 100% sequence identity to SEQ ID NO: 98, 327, or 378; (ii) the extracellular domain can comprise amino acids having at least 90%, 91%, 92%, 93%, 94%, 95%, 96%, 97%, 98%, 99% or 100% sequence identity to one of SEQ ID NOs: 354-363, or the extracellular domain can be encoded by a polynucleotide having at least 90%, 91%, 92%, 93%, 94%, 95%, 96%, 97%, 98%, 99% or 100% sequence identity to one of SEQ ID NOs: 364-373; (iii) the hinge region can comprise amino acids having at least 90%, 91%, 92%, 93%, 94%, 95%, 96%, 97%, 98%, 99% or 100% sequence identity to SEQ ID NO: 21, 22, 288, 289, 319, or 321, or the hinge region can be encoded by a polynucleotide having at least 90%, 91%, 92%, 93%, 94%, 95%, 96%, 97%, 98%, 99% or 100% sequence identity to SEQ ID NO: 315-318, 320, or 322; (iv) the one or more transmembrane domains each comprise amino acids having at least 90%, 91%, 92%, 93%, 94%, 95%, 96%, 97%, 98%, 99% or 100% sequence identity to a sequence independently selected from the group consisting of SEQ ID NOs: 23, 24, 290, 291, 323, and 325, or the one or more transmembrane domains can be each encoded by a polynucleotide having at least 90%, 91%, 92%, 93%, 94%, 95%, 96%, 97%, 98%, 99% or 100% sequence identity to a sequence independently selected from the group consisting of SEQ ID NOs: 324, 326, and 374-377; (v) the intracellular signaling domain can comprise amino acids having at least 90%, 91%, 92%, 93%, 94%, 95%, 96%, 97%, 98%, 99% or 100% sequence identity to one or more of SEQ ID NOs: 6, 8, and 267-287, or the intracellular signaling domain can be encoded by a polynucleotide having at least 90%, 91%, 92%, 93%, 94%, 95%, 96%, 97%, 98%, 99% or 100% sequence identity to one or more of SEQ ID NO: 266, and 293-314. In certain embodiments, in the iCAR: (i) the signal peptide can comprise amino acids having the sequence of SEQ ID NO: 97 or 292, or the signal peptide can be encoded by a polynucleotide sequence of SEQ ID NO: 98, 327, or 378; (ii) the extracellular domain can comprise amino acids having the sequence of SEQ ID NOs: 354-363, or the extracellular domain can be encoded by the polynucleotide sequence of SEQ ID NO: 364-373; (iii) the hinge region can comprise amino acids having the sequence of SEQ ID NO: 21, 22, 288, 289, 319, or 321, or the hinge region can be encoded by a polynucleotide sequence of SEQ ID NO: 315-318, 320, or 322; (iv) the one or more transmembrane domains each comprise amino acids having a sequence independently selected from the group consisting of SEQ ID NO: 23, 24, 290, 291, 323, and 325, or the one or more transmembrane domains can be each encoded by a polynucleotide having a sequence independently selected from the group consisting of SEQ ID NOs: 324, 326, and 374-377; and (v) the intracellular signaling domain can comprise amino acids having the sequence of one or more of SEQ ID NOs: 6, 8, and 267-287, or the intracellular signaling domain can be encoded by the polynucleotide of one of SEQ ID NOs: 266, and 293-314. In certain embodiments, the derivative cell can be a natural killer (NK) cell or a T cell. In certain embodiments, the derivative cell can be a T cell. In certain embodiments, the T cell can be a gamma delta T cell. In certain embodiments, the T cell can be a gamma delta Vγ9/Vδ1 T cell.

In some aspects, the present disclosure provides a composition comprising a derivative cell of the present disclosure. In certain embodiments, the composition can further comprise or can be used in combination with, one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD).

In some aspects, the present disclosure provides a CD34+ hematopoietic progenitor cell (HPC) derived from an induced pluripotent stem cell (iPSC) of the present disclosure. In certain embodiments, the CAR can be a dual-targeting CAR comprising an additional antigen-binding domain that specifically binds to an antigen selected from the group consisting of CD70, Folate Receptor alpha, FSHR, mesothelin, and SLITRK6. In certain embodiments, the one or more exogenous polynucleotides encode an additional CAR comprising an antigen-binding domain that specifically binds to an antigen selected from the group consisting of CD70, Folate Receptor alpha, FSHR, mesothelin, and SLITRK6. In certain embodiments, CD34+ HPC can further comprise an exogenous polynucleotide encoding a human leukocyte antigen E (HLA-E) and/or human leukocyte antigen G (HLA-G). In certain embodiments, one or more of the exogenous polynucleotides can be integrated at one or more loci on the chromosome of the cell independently selected from the group consisting of AAVS1, CLYBL, CCR5, ROSA26, collagen, HTRP, Hl 1, GAPDH, RUNX1, B2M, TAPI, TAP2, Tapasin, NLRC5, RFXANK, CIITA, RFX5, RFXAP, TCR a or b constant region, NKG2A, NKG2D, CD33, CD38, CIS, CBL-B, SOCS2, PD1, CTLA4, LAG3, TIM3, and TIGIT genes, provided at least one of the exogenous polynucleotides can be integrated at a locus of a gene selected from the group consisting of B2M, TAP 1, TAP 2, Tapasin, RFXANK, CIITA, RFX5 and RFXAP genes to thereby result in a deletion or reduced expression of the gene. In certain embodiments, one or more of the exogenous polynucleotides can be integrated at the loci of the AAVS1 and B2M genes. In certain embodiments, the CD34+ HPC can have a deletion or reduced expression of one or more of B2M or CIITA genes. In certain embodiments, the CAR can comprise: (i) a signal peptide; (ii) an extracellular domain comprising a binding domain that specifically binds the Nectin4 antigen; (iii) a hinge region; (iv) a transmembrane domain; (v) an intracellular signaling domain; and (vi) a co-stimulatory domain. In certain embodiments, the extracellular domain can comprise a VHH single domain antibody that specifically binds the Nectin4 antigen. In certain embodiments, the extracellular domain can comprise amino acids having at least 90%, 91%, 92%, 93%, 94%, 95%, 96%, 97%, 98%, 99% or 100% sequence identity to one of SEQ ID NOs: 105-130. In certain embodiments, the CD34+ HPC can comprise an additional CAR comprising: (i) a signal peptide; (ii) an additional extracellular domain comprising a binding domain that specifically binds an antigen selected from the group consisting of CD70, Folate Receptor alpha, FSHR, mesothelin, and SLITRK6; (iii) a hinge region; (iv) a transmembrane domain; (v) an intracellular signaling domain; and (vi) a co-stimulatory domain, such as a co-stimulatory domain comprising a CD28 signaling domain. In certain embodiments, the additional extracellular domain can comprise a VHH that specifically binds the antigen selected from the group consisting of CD70, Folate Receptor alpha, FSHR, mesothelin, and SLITRK6. In certain embodiments, the CD34+ HPC can comprise an additional exogenous polynucleotide encoding a CD16 protein and an NKG2D protein, wherein the CD16 protein and the NKG2D protein can be operably linked by an autoprotease peptide. In certain embodiments, the CD16 protein can be a CD16 variant protein. In certain embodiments, the CD16 variant can be a high affinity CD16 variant. In certain embodiments, the CD16 variant can be a non-cleavable CD16 variant. In certain embodiments, the CD16 variant can comprise one or more amino acid substitutions selected from the group consisting of F158V, F176V, S197P, D205A, S219A, T220A, and any combination thereof.

In some aspects, the present disclosure provides a chimeric antigen receptor (CAR) polypeptide comprising an extracellular domain comprising an antigen binding domain that specifically binds to Nectin4. In certain embodiments, the CAR can be a dual-targeting CAR, and wherein the extracellular domain can comprise an additional antigen-binding domain that specifically binds to an antigen selected from the group consisting of CD70, Folate Receptor alpha, FSHR, mesothelin, and SLITRK6. In certain embodiments, the CAR can comprise: (i) a signal peptide; (ii) the extracellular domain comprising the antigen binding domain that specifically binds to the Nectin4 antigen; (iii) a hinge region; (iv) one or more transmembrane domains; (v) an intracellular signaling domain; and/or (vi) a co-stimulatory domain. In certain embodiments, the extracellular domain can comprise a VHH single domain antibody that specifically binds to the Nectin4 antigen. In certain embodiments, the extracellular domain can comprise amino acids having at least 90%, 91%, 92%, 93%, 94%, 95%, 96%, 97%, 98%, 99% or 100% sequence identity to one of SEQ ID NOs: 105-130. In certain embodiments, the extracellular domain can be encoded by a polynucleotide having at least 90%, 91%, 92%, 93%, 94%, 95%, 96%, 97%, 98%, 99% or 100% sequence identity to one of SEQ ID NOs: 131-156. In certain embodiments, the CAR can be encoded by a polynucleotide having at least 90%, 91%, 92%, 93%, 94%, 95%, 96%, 97%, 98%, 99% or 100% sequence identity to one of SEQ ID NOs: 171-184. In certain embodiments, the signal peptide can comprise a GMCSFR signal peptide or a MARS signal peptide. In certain embodiments, the hinge region for each of the CAR and the additional CAR can be independently selected from the group consisting of a CD28 hinge region, an IgG4 hinge region, and a CD8 hinge region. In certain embodiments, the transmembrane domain for each of the CAR and the additional CAR can be independently selected from the group consisting of a CD28 transmembrane domain and a CD8 transmembrane domain. In certain embodiments, the intracellular signaling domain can comprise a CD3ζ intracellular domain. In certain embodiments, the co-stimulatory domain for each of the CAR and the additional CAR can be independently selected from the group consisting of a CD28 signaling domain, a 41BB signaling domain, and a DAP10 signaling domain. In certain embodiments, in the CAR: (i) the signal peptide can comprise amino acids having at least 90%, 91%, 92%, 93%, 94%, 95%, 96%, 97%, 98%, 99% or 100% sequence identity to SEQ ID NOs: 1, 97, or 98; (ii) the extracellular domain can comprise amino acids having at least 90%, 91%, 92%, 93%, 94%, 95%, 96%, 97%, 98%, 99% or 100% sequence identity to SEQ ID NOs: 105-130, or the extracellular domain can be encoded by a polynucleotide having at least 90%, 91%, 92%, 93%, 94%, 95%, 96%, 97%, 98%, 99% or 100% sequence identity to SEQ ID NOs: 131-156; (iii) the hinge region can comprise amino acids having at least 90%, 91%, 92%, 93%, 94%, 95%, 96%, 97%, 98%, 99% or 100% sequence identity to SEQ ID NO: 21 or 96; (iv) the transmembrane domain can comprise amino acids having at least 90%, 91%, 92%, 93%, 94%, 95%, 96%, 97%, 98%, 99% or 100% sequence identity to SEQ ID NO: 23 or 24; (v) the intracellular signaling domain can comprise amino acids having at least 90%, 91%, 92%, 93%, 94%, 95%, 96%, 97%, 98%, 99% or 100% sequence identity to SEQ ID NO: 6, or the intracellular signaling domain can be encoded by a polynucleotide having at least 90%, 91%, 92%, 93%, 94%, 95%, 96%, 97%, 98%, 99% or 100% sequence identity to SEQ ID NO: 101; and (vi) the co-stimulatory domain can comprise amino acids having at least 90%, 91%, 92%, 93%, 94%, 95%, 96%, 97%, 98%, 99% or 100% sequence identity to SEQ ID NO: 8 or 17. In certain embodiments, in the CAR: (i) the signal peptide can comprise amino acids having the sequence of SEQ ID NO: 1, 97, or 98; (ii) the extracellular domain can comprise amino acids having the sequence of one of SEQ ID NOs: 105-130; (iii) the hinge region can comprise amino acids having the sequence of SEQ ID NO: 21 or 96; (iv) the transmembrane domain can comprise amino acids having the sequence of SEQ ID NO: 23 or 24; (vi) the intracellular signaling domain can comprise amino acids having the sequence of SEQ ID NO: 6, or the intracellular signaling domain can be encoded by the polynucleotide of SEQ ID NO: 101; and (vii) the co-stimulatory domain can comprise amino acids having the sequence of SEQ ID NO: 8 or 17.

In some aspects, the present disclosure provides an inhibitory chimeric antigen receptor (iCAR) polypeptide comprising an extracellular domain comprising an antigen binding domain that specifically binds at least one antigen selected from the group consisting of Adrenoceptor Beta 2 (ADRB2), Aquaporin 4 (AQP4), Claudin 10 (CLDN10B), Desmocollin (DSC) 1, DSC3, Desmoglein (DSG) 1, DSG3, Glycerophosphodiester Phosphodiesterase Domain Containing 2 (GDPD2), Hydroxycarboxylic Acid Receptor 3 (HCAR3), Lymphocyte Antigen 6 Family Member D (LY6D), V-Set And Immunoglobulin Domain Containing 8 (VSIG8). In certain embodiments, the iCAR can comprise: (i) a signal peptide; (ii) the extracellular domain comprising the antigen binding domain that specifically binds at least one antigen selected from the group consisting of Adrenoceptor Beta 2 (ADRB2), Aquaporin 4 (AQP4), Claudin 10 (CLDN10B), Desmocollin (DSC) 1, DSC3, Desmoglein (DSG) 1, DSG3, Glycerophosphodiester Phosphodiesterase Domain Containing 2 (GDPD2), Hydroxycarboxylic Acid Receptor 3 (HCAR3), Lymphocyte Antigen 6 Family Member D (LY6D), V-Set And Immunoglobulin Domain Containing 8 (VSIG8); (iii) a hinge region; (iv) one or more transmembrane domains; (v) an intracellular signaling domain; and/or (vi) a co-stimulatory domain. In certain embodiments, the antigen binding domain specifically binds at least one antigen selected from DSC1, DSC3, DSG1, and DSG3. In certain embodiments, the antigen binding domain specifically binds to DSG1. In certain embodiments, the antigen binding domain specifically binds to (i) DSG1, and (ii) at least one antigen selected from DSC1, DSC3, and DSG3. In certain embodiments, the extracellular domain of the iCAR can comprise amino acids having at least 90%, 91%, 92%, 93%, 94%, 95%, 96%, 97%, 98%, 99% or 100% sequence identity to one or more of SEQ ID NOs: 354-363. In certain embodiments, the extracellular domain of the iCAR can be encoded by a polynucleotide having at least 90%, 91%, 92%, 93%, 94%, 95%, 96%, 97%, 98%, 99% or 100% sequence identity to one or more of SEQ ID NOs: 364-373. In certain embodiments, the signal peptide of the iCAR can comprise a CD8 signal peptide, a GMCSFR signal peptide, a MARS signal peptide, or an IgK signal peptide or variant thereof. In certain embodiments, the signal peptide of the iCAR can comprise amino acids having at least 90%, 91%, 92%, 93%, 94%, 95%, 96%, 97%, 98%, 99% or 100% sequence identity to one of SEQ ID NOs: 97 and 292. In certain embodiments, the signal peptide of the iCAR can be encoded by a polynucleotide having at least 90%, 91%, 92%, 93%, 94%, 95%, 96%, 97%, 98%, 99% or 100% sequence identity to one of SEQ ID NOs: 98, 327, and 378. In certain embodiments, in the iCAR: (i) the signal peptide can comprise amino acids having at least 90%, 91%, 92%, 93%, 94%, 95%, 96%, 97%, 98%, 99% or 100% sequence identity to SEQ ID NOs: 97 or 292; (ii) the extracellular domain can comprise amino acids having at least 90%, 91%, 92%, 93%, 94%, 95%, 96%, 97%, 98%, 99% or 100% sequence identity to SEQ ID NOs: 354-363, or the extracellular domain can be encoded by a polynucleotide having at least 90%, 91%, 92%, 93%, 94%, 95%, 96%, 97%, 98%, 99% or 100% sequence identity to SEQ ID NOs: 364-373; and (iii) the iCAR can comprise amino acids having at least 90%, 91%, 92%, 93%, 94%, 95%, 96%, 97%, 98%, 99% or 100% sequence identity to SEQ ID NO: 242-265, and 352, or the iCAR can comprise a sequence of amino acids encoded by a polynucleotide having at least 90%, 91%, 92%, 93%, 94%, 95%, 96%, 97%, 98%, 99% or 100% sequence identity to one of SEQ ID NO: 328-351, and 353. In certain embodiments, in the iCAR: (i) the signal peptide can comprise amino acids having the sequence of SEQ ID NO: 97, or 292; (ii) the extracellular domain can comprise amino acids having the sequence of one of SEQ ID NOs: 354-363; and/or (iii) the iCAR can comprise amino acids having the sequence of one of SEQ ID NO: 242-265, and 352.

In some aspects, the present disclosure provides an induced pluripotent stem cell (iPSC) or a derivative cell thereof of the present disclosure, and further can comprise an iCAR of the present disclosure. In some aspects, the present disclosure provides a pharmaceutical composition comprising a derivative of the present disclosure.

In some aspects, the present disclosure provides a method of treating cancer in a subject in need thereof, comprising administering a derivative cell of the present disclosure, or a composition of the present disclosure, to a subject in need thereof. In certain embodiments, the cancer can be selected from the group consisting of leukemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovarian cancer, prostate cancer, lung cancer, colorectal cancer, cervical cancer, liver cancer, head and neck cancer, esophageal cancer, pancreatic cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP). In certain embodiments, the cancer can be selected from the group consisting of bladder, breast, lung, pancreatic, ovarian, head & neck, and esophageal cancers. In certain embodiments, the subject has minimal residual disease (MRD) after an initial cancer treatment. In certain embodiments, the subject has no minimal residual disease (MRD) after one or more cancer treatments or repeated dosing. In certain embodiments, the method can further comprise administering to the subject a therapeutic agent selected from the group consisting of ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, avelumab, ofatumumab, panitumumab, and ustekinumab. In certain embodiments, the method can further comprise administering to the subject a therapeutic agent, wherein the therapeutic agent can be avelumab. In certain embodiments, the cell and the therapeutic agent can be administered concurrently. In certain embodiments, the cell and the therapeutic agent can be administered sequentially.

In some aspects, the present disclosure provides a method of manufacturing a derivative cell of the present disclosure, comprising differentiating an iPSC of the present disclosure under conditions for cell differentiation to thereby obtain the derivative cell. In certain embodiments, the iPSC can be obtained by genetically engineering an unmodified iPSC, wherein the genetic engineering can comprise targeted editing of the genome of the iPSC. In certain embodiments, the targeted editing can comprise deletion, insertion, or in/del carried out by CRISPR, ZFN, TALEN, homing nuclease, homology recombination, or any other functional variation of these methods.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-C show (A) a diagram of an exemplary cell of the present disclosure, which expresses an anti-Nectin4 CAR on the cell surface; (B) a diagram of an exemplary cell of the present disclosure targeting a tumor cell using (i) an anti-Nectin4 CAR to bind a Nectin4 antigen on the tumor cell surface, and (ii) surface-expressed CD16 to bind to a tumor antigen antibody (e.g., cetuximab, trastuzumab, avelumab, and/or others) or antibodies that modulate the tumor microenvironment such as antibodies against checkpoint inhibitors including PD-L1 and CTLA4 (avelumab, ipilimumab, and/or others); and (C) a table of exemplary genetic edits performed on a cell of the present disclosure, and the associated rationale for performing the genetic edit.

FIGS. 2A-C show (A) full length Nectin4, (B) IgC1/2 Nectin4 domains, and (C) IgC2 Nectin4 domains used for VHH & scFv binder discovery.

FIGS. 3A-C show (A) a diagram of (left) human IgG, which is the source of certain scFv binders of the present disclosure, and (right) llama IgG, which is the source of certain VHH binders of the present disclosure; (B) a diagram of the construction of a VHH library, where V2.0 phage libraries containing ~2×10¹⁰ unique sequences were constructed; and (C) phage panning against Nectin4 protein. Three rounds of phage panning on a VHH phage library were performed using plate-bound Nectin4-HIS protein (AcroBiosystems #NE4-H52H3), and individual colonies were screened by ELISA using periplasmic extract (PPE).

FIG. 4 shows a flow chart detailing the VHH CAR selection process. VHH binders were selected using biophysical analysis, cell binding (fluorescence activated cell sorting; FACS) assays, Nurkat tonic/activation, in vitro cytotoxicity assays, and/or Retrogenix/in vivo screening.

FIGS. 5A-H show Nectin4 cell surface expression on (A) HeLa cells, (B) K562 cells, (C) CHO-K1 cells, (D) HEPG2 cells, (E) T-47D cells, (F) OVCAR3 cells, (G) OE19 cells, (H) A431 cells, and (G) CHO-Nectin4 cells. Flow cytometry was used to detect Nectin4 protein expression on the cell surface of a panel of normal and tumor cell lines using the PE anti-Nectin4 detection antibody (R & D Systems, Cat #FAB2659P, clone:337516, msIgG2b; 1 ug/ml). Panel (H) shows a summary table of target cells lines, a description thereof, the percentage of Nectin4 positive cells, and the Nectin4 mean fluorescence intensity (MFI ratio).

FIG. 6 shows results of 14 anti-Nectin4 VHH-Fc were screened for binding to Nectin4 positive cell lines. All 14 VHH-Fc demonstrated specific binding to CHO-Nectin4 cells, and 12 of 14 cell lines demonstrated specific binding to the Nectin4 positive tumor cell line T47D.

FIG. 7 shows a diagram of the Nurkat activation assay. Nur77-sfGFP-PEST KI Jurkat reporter line (Nurkat cells) was engineered with lentiviral transduction to drive GFP expression from the Nur77 promoter with a panel of VHH CARs directed against Nectin4. Nurkat cells were co-cultured with target cells without Nectin4 or with varying densities of Nectin4 on the cell surface. Flow cytometry was used to quantify the GFP signal that resulted from Nurkat cell activation through the CAR.

FIGS. 8A-D show (A) a schematic of an exemplary anti-Nectin4 VHH CAR of the present disclosure; and (B) results of a tonic signaling assay as a function of CAR Higher levels of CAR on the cell surface may lead to artificially higher tonic signaling; (C) results of a Jurkat_Nur77 Reporter assay for activation via Nectin4 negative or positive cell lines; and (D) results of a Jurkat_Nur77 Reporter assay for tonic signaling via Nectin4 negative or positive cell lines.

FIG. 9 shows results of VHH-CAR T-cell-mediated, target-specific killing of Nectin4 positive K562, HeLa, T47D, OVCAR3, OE19, A431 cell lines in vitro.

FIG. 10 shows results of T-cell activation with 1:1 co-culture of cells of the present disclosure expressing anti-Nectin4 CARs with various target cells, as measured by (top) percentage of cells that are CD25 positive and (bottom) IL-2 expression.

FIGS. 11A-B show (A) a table of binding affinity to human and mouse Nectin4 and target epitopes for various anti-Nectin4 binders of the present disclosure; and (B) a diagram showing the binding of various anti-Nectin4 binders to Nectin4.

FIGS. 12A-B show (A) results of a Nectin4 binding specificity study where VHH_Fc protein binding to A431, A549, Capan-2, HEPG2, Jurkat, MOLM-13, NALM-6, OE19, OVCAR3, U-2 OS cell lines was determined. A cell-based specificity FACS screen was established to support lead VHH characterization and selection. VHH-Fc cell binding dose-response curves (DRC) are generated against a diverse panel of human cell lines derived from various tissue/organ types, and VHH-Fc that demonstrate non-specific binding to target-negative lines are flagged for potential off-target binding, while VHH-Fc that demonstrate minimal non-specific binding to target-negative lines can be prioritized as lead candidates; and (B) a table of target cell lines used in the binding specificity screen.

FIGS. 13A-B show (A) Nectin4 antigen density, which was assessed on a variety of solid tumor and control cell lines, as well as primary human keratinocytes (PHKs), using Quantibrite PE (Beckton Dickinson). Quantibrite beads were coated with 4 calculated levels of PE, low, medium low, medium high, and high. Using these calculated PE/bead values and their fluorescence intensity in flow cytometry, a standard curve was created to estimate the antigen density on various target cell lines; and (B) anti-Nectin4 phycoerythrin (PE)/cell (normalized to isotype) for various target cell lines, which are segregated into groups of cells that are Nectin4 negative, or having low, medium, or high levels of Nectin4.

FIGS. 14A-H show cytotoxicity of Nectin4 CARs across varying effector to target cell ratios for (A) TUCCSUP cells, (B) HELA cells, (C) HT1197 cells, (D) T24 cells, (E) HT1376 cells, (F) OVCAR cells, (G) OE19 cells, and (H) T47D cells. The T24 tumor cell line expresses similar levels of Nectin4 compared to primary human keratinocytes. This line is being used as a surrogate for Nectin4 expression in human keratinocytes to help select binders that kill tumors without significant skin toxicities.

FIGS. 15A-D show (A) cumulative cytotoxicity as a percentage of target cells killed, (B) cumulative interferon gamma (IFN) secretion, and (C) cumulative IL2 secretion for effector cells expressing various anti-Nectin4 VHH CARs.

FIGS. 16A-C show (A) cumulative cytotoxicity as a percentage of target cells killed, (B) cumulative interferon gamma (IFN) secretion, and (C) cumulative IL2 secretion for effector cells expressing various anti-Nectin4 VHH CARs having either 41BB or CD28 costimulatory domains.

FIGS. 17A-C show the results of efficacy screening of primary T-cells expressing anti-Nectin4 VHH CARs in the OVCAR-3 xenograft tumor model, including (A) tumor burden, (B) mouse body weight change, and (C) percentage of CAR positive cells.

FIGS. 18A-B show (A) all single cell types with NECTIN4 gene expression greater than 50 transcripts per million across 30 tissues and (B) all single cell types in bronchus or lung tissues with NECTIN4 gene expression greater than 10 transcripts per million. Data is from the publicly available Human Protein Atlas single cell RNA sequencing atlas of normal tissue.

FIGS. 19A-B show gene expression in normal skin tissue of (A) suprabasal keratinocyte cells (B) basal keratinocyte cells compared to median tumor gene expression across patients with bladder, breast, esophagus, head and neck, non-small cell lung, ovary, or pancreas cancer indications. Median patient gene expression is calculated from bulk RNA sequencing measurements of human tumors from the The Cancer Genome Atlas Program. Suprabasal keratinocyte cell and basal keratinocyte cell gene expression are calculated from the Human Protein Atlas single cell RNA sequencing atlas of normal tissue. Only surfaceome genes coding proteins of the plasma membrane which are at least partially exposed to the extracellular space are shown. DSG1 is highlighted in blue to emphasize its high gene expression in normal suprabasal and basal skin keratinocytes and low gene expression across most of the cancer indications displayed.

FIG. 20A-B show (A) the geometric mean of patient gene expression across bladder, breast, esophagus, head and neck, non-small cell lung, ovary, or pancreas cancer indications compared to the geometric mean of suprabasal and basal keratinocyte gene expression in skin. Gene expression is expressed in transcripts per million. Only surfaceome genes coding proteins of the plasma membrane which are at least partially exposed to the extracellular space are shown; and (B) candidate gene targets for an inhibitory CAR preventing lysis of skin keratinocytes. The Tumor TPM column shows median patient gene expression calculated from bulk RNA sequencing measurements of human tumors from The Cancer Genome Atlas Program. Patients with bladder, breast, esophagus, head and neck, non-small cell lung, ovary, or pancreas cancer indications are included. The keratinocyte TPM column displays the mean of skin suprabasal keratinocyte cell and basal keratinocyte cell gene expression from the Human Protein Atlas single cell RNA sequencing atlas of normal tissue. The Fold Difference column shows the “Keratinocyte TPM” column divided by the “Tumor TPM” column. Genes are arranged in order of descending Fold Difference with all surfaceome genes with greater than 50 fold difference displayed. The Expected Cell Type column annotates the single cell types across 30 tissue with the greatest expression of this gene as measured by the Human Protein Atlas single cell RNA sequencing dataset. All displayed genes are part of the surfaceome genes coding proteins of the plasma membrane which are at least partially exposed to the extracellular space are shown. However, some genes may also localize to other membranes of the cell. The Expected Subcellular Localization column annotates information about the expected subcellular localization of each gene coded protein from the Human Protein Atlas. The Protein Data column shows annotations from additional data sources as to the protein expression and surface display in skin keratinocyte cells. The Notes column displays an analysis of the relative detection levels of the gene coded protein across skin keratinocytes in different layers of skin and across cell types of the body. Shaded rows indicate those genes with greatest likelihood of being displayed as protein on the surface of skin keratinocytes per this analysis.

FIG. 21 shows the distribution of tumor gene expression across patients in The Cancer Genome Atlas for the genes ADRB2, DSC1, DSC3, DSG1, DSG3, GDPD2, HCAR3, LY6D, NECTIN4, and VSIG8. Results are shown separately for bladder, breast, esophagus, head and neck, non-small cell lung, ovary, and pancreas cancer indications. The number of patients included for each indication is displayed under the indication name (n=number of patients). Gene expression is displayed in units of transcripts per million from bulk RNA sequencing.

FIG. 22 shows the distribution of tumor gene expression across patients in The Cancer Genome Atlas dataset for the genes DSG1 and NECTIN4. Results are shown separately for bladder, breast, esophagus, head and neck, non-small cell lung, ovary, and pancreas cancer indications. The number of patients included for each indication is displayed under the indication name (n=number of patients). Gene expression is displayed in units of transcripts per million from bulk RNA sequencing.

FIG. 23 shows the tumor gene co-expression of DSG1 and NECTIN4 for each patient in The Cancer Genome Atlas for bladder, breast, esophagus, head & neck, non-small cell lung, ovary, and pancreas cancer indications. The number of patients included for each indication is displayed under the indication name (n=number of patients). Gene expression is displayed in units of transcripts per million from bulk RNA sequencing.

FIG. 24 shows tumor NECTIN4 and DSG1 protein expression across patients in The Human Protein Atlas as measured by immunohistochemistry protein microarrays and graded by pathologists as not detected, low, medium, or high expression. Results are plotted separately for patients according to their cancer indication. Between 4 and 12 patients are included in each cancer indication, as shown by the length of the bar for that indication. Some potential cancer indications for NECTIN4 targeting therapy are indicated with arrows. Note that DSG1 is only detected in skin cancer, head and neck cancer, and one lung cancer patient. Here lung cancer could include both small cell lung and non-small cell lung cancer indications.

FIG. 25 shows DSG1 and NECTIN4 gene expression in normal tissues in the Genotype-Tissue Expression project as measured by bulk RNA sequencing in units of transcripts per million. Box plots render the 25 percentile through 75 percentile of gene expression across patients with a mark displaying the median. Whiskers on the box plot are the length of 1.5 interquartile distances and outlier patients outside this range are displayed with a point. The number of individuals for each tissue are indicated (n=number of individuals). DSG1 and NECTIN4 have consistently high gene expression in both sun-exposed and not-sun-exposed skin across individuals that exceeds the gene expression in any other normal tissue.

FIG. 26 show gene expression of DSG1, NECTIN4, and PRF1 in several lines of induced pluripotent stem cells differentiated into T cells. Gene expression is displayed in units of transcripts per million as measured by bulk RNA sequencing. With PRF1 as a reference, DSG1 and NECTIN4 gene expression is very low (<1 transcript per million) for all T cells differentiated by induced pluripotent stem cells. Gene expression is shown for both day 28 of T cell differentiation (D28) and those at day 35 (D35-aAPC) that have been cultured with irradiated artificial antigen presenting cells.

FIG. 27 shows NECTIN4 and DSG1 gene expression in cross-tissue cell type clusters of The Human Protein atlas single cell RNA sequencing dataset on 30 normal human tissues. Only those cell types with NECTIN4 gene expression greater than 10 Transcripts Per Million are displayed. The tissues where each cell type are found in the dataset are annotated on the right side of the plot. A dotted vertical line is displayed at 10 transcripts per million.

FIGS. 28A-B show (A) AQP4, DSG1, and NECTIN4 gene expression in all cell type clusters in skin (left) and lung (right) tissue samples in the Human Protein Atlas single cell RNA sequencing dataset. The relative abundance of each cell type in each tissue is annotated with a percentage next to the cluster name. Gene expression is displayed in united of transcripts per million; and (B) information from multiple sources showing that DSG1 or alternative desmosome gene, CLDN10B, and AQP4 are ideal targets for an inhibitory CAR for NECTIN4 therapy. Note that each inhibitory CAR target is suited for preventing lysis of different normal cell types from different tissues of the body.

FIG. 29 shows NECTIN4 protein expression for cell type in each normal tissue in The Human Protein Atlas as measured by immunohistochemistry protein microarrays and graded by pathologists as not detected, low, medium, or high expression. The cell types in normal tissues with the greatest detected NECTIN4 protein expression by this method are outlined at the top of the plot.

FIG. 30 shows tumor NECTIN4 protein expression across patients for all cancer indications in The Human Protein Atlas as measured by immunohistochemistry protein microarrays and graded by pathologists as not detected, low, medium, or high expression. Results are plotted separately for patients according to their cancer indication. Between 4 and 12 patients are included in each cancer indication, as shown by the length of the bar for that indication.

FIG. 31 shows DSG1 and NECTIN4 gene expression in tumor tissues in the The Cancer Genome Atlas Genome Atlas as measured by bulk RNA sequencing in units of transcripts per million. Box plots render the 25 percentile through 75 percentile of gene expression across patients with a mark displaying the median. Whiskers on the box plot are the length of 1.5 interquartile distances and outlier patients outside this range are displayed with a point. The number of individuals for each cancer indication are indicated (n=number of individuals).

FIGS. 32A-C show patient-wise gene co-expression of DSG1 and NECTIN4 in The Cancer Genome Project in (A) tumors from patients with lung cancer indications including small cell and non-small cell and (B) tumors from select NECTIN4 expressing cancer indications, and (C) all normal solid tissue data from cancer patients in the dataset. Gene expression is displayed in units of transcripts per million from bulk RNA sequencing. Note that the number of samples available for the normal solid tissue data is indicated by n (n=number of samples).

FIGS. 33A-C show gene expression in normal tissue from the Genotype-Tissue Expression Project as measured by bulk RNA sequencing in units of transcripts per million for (A) DSC3 and NECTIN4, (B) CLCA4 and NECTIN4, and (C) DSC1 and NECTIN4. Box plots render the 25 percentile through 75 percentile of gene expression across patients with a mark displaying the median. Whiskers on the box plot are the length of 1.5 interquartile distances and outlier patients outside this range are displayed with a point. The number of individuals for each tissue is indicated (n=number of individuals).

FIG. 34 shows NECTIN4 gene expression for single cell clusters from 30 normal tissues in the Human Protein Atlas single cell RNA sequencing dataset for which a combinatorial inhibitory CAR may provide protection from a NECTIN4 targeting activating CAR. Only those single cell clusters with NECTIN4 greater than 50 transcripts per million and DSG1 less than 50 transcripts per million are displayed.

FIG. 35 shows median tumor gene expression across patients for bladder, breast, esophagus, head and neck, non-small cell lung, ovary, or pancreas cancer indications compared to a weighted geometric mean of the gene expression of the normal tissue cell types with NECTIN4>50 and DSG1<50 transcripts per million displayed in FIG. 35. Median patient gene expression is calculated from bulk RNA sequencing measurements of human tumors from The Cancer Genome Atlas Program. The weighted geometric mean of normal tissue cell types is calculated from the Human Protein Atlas single cell RNA sequencing atlas of 30 normal tissues. The weighting is computed for each cell type and gene as follows: (weighted transcripts per million)=(transcripts per million)*(relative abundance of this lung cell type among all lung cells)*(NECTIN4 transcripts per million)/(this gene transcripts per million) Only surfaceome genes coding proteins of the plasma membrane which are at least partially exposed to the extracellular space are shown. CLDN10 is highlighted in blue to emphasize its high gene expression in normal NECTIN4 high/DSG1 low expressing cell types and its low gene expression across most of the cancer indications displayed.

FIG. 36 shows the geometric mean of patient tumor gene expression shown in FIG. 37 including bladder, breast, esophagus, head and neck, non-small cell lung, ovary, or pancreas cancer indications compared to a geometric mean of the gene expression of the normal tissue cell types with NECTIN4>50 and DSG1<50 transcripts per million displayed in FIG. 35. Median patient gene expression is calculated from bulk RNA sequencing measurements of human tumors from The Cancer Genome Atlas Program. The weighted geometric mean of normal lung cell types is calculated from the Human Protein Atlas single cell RNA sequencing atlas of normal tissue. The weighting is computed for each cell type and gene as follows: (weighted transcripts per million)=(transcripts per million)*(relative abundance of this lung cell type among all lung cells)*(NECTIN4 transcripts per million)/(this gene transcripts per million). Only surfaceome genes coding proteins of the plasma membrane which are at least partially exposed to the extracellular space are shown. CLDN10 is highlighted in blue to emphasize its high gene expression in normal NECTIN4 high/DSG1 low cell types and low gene expression in cancer.

FIG. 37 shows the distribution of tumor gene expression across patients in The Cancer Genome Atlas dataset for the genes CLDN10 and NECTIN4. Results are shown separately for bladder, breast, esophagus, head and neck, non-small cell lung, ovary, and pancreas cancer indications. The number of patients included for each indication is displayed under the indication name (n=number of patients). Gene expression is displayed in units of transcripts per million from bulk RNA sequencing.

FIG. 38 shows CLDN10 and NECTIN4 gene expression in normal tissues in the Genotype-Tissue Expression project as measured by bulk RNA sequencing in units of transcripts per million. Box plots render the 25 percentile through 75 percentile of gene expression across patients with a mark displaying the median. Whiskers on the box plot are the length of 1.5 interquartile distances and outlier patients outside this range are displayed with a point. The number of individuals for each tissue are indicated (n=number of individuals).

FIGS. 39A-E show the gene coexpression of DSG1 and/or CLDN10 in NECTIN4 expressing cell types in 30 normal tissues as measured by single cell RNA sequencing. Shown are all single cell type clusters with (A) NECTIN4>100 transcripts per million and (DSG1 or CLDN10>100 transcripts per million). (B) NECTIN4>50 transcripts per million and (DSG1 or CLDN10>50 transcripts per million), (C) NECTIN4>20 transcripts per million and (DSG1 or CLDN10>20 transcripts per million), (D) NECTIN4>10 transcripts per million and (DSG1 or CLDN10>10 transcripts per million), and (E) NECTIN4 greater than the indicated transcripts per million and DSG1 and CLDN10 less than the indicated transcripts per million. Data is from the publicly available Human Protein Atlas single RNA sequencing atlas of normal tissue.

FIG. 40 show gene expression of AQP4, CLDN10, DSG1, and NECTIN4 in several lines of induced pluripotent stem cells differentiated into T cells. Gene expression is displayed in units of transcripts per million as measured by bulk RNA sequencing. Gene expression of all genes is very low (<1 transcript per million) for all samples. Gene expression is shown for both day 28 of T cell differentiation (D28) and those at day 35 (D35-aAPC) that have been cultured with irradiated artificial antigen presenting cells.

FIG. 41 shows tumor NECTIN4, DSG1, DSC1, ADRB2, DSC3, LY6D, DSG3, and CLCA4 protein expression in patient tumors in The Human Protein Atlas as measured by immunohistochemistry protein microarrays and graded by pathologists as not detected, low, medium, or high expression. Results are plotted separately for patients according to their cancer indication. Between 4 and 12 patients are included in each cancer indication, as shown by the length of the bar for that indication.

FIG. 42 show median tumor gene expression across patients for bladder, breast, esophagus, head and neck, non-small cell lung, ovary, or pancreas cancer indications compared to a weighted geometric mean of the gene expression of normal lung cell types with NECTIN4 gene expression greater than 10 (as shown in FIG. 18B, excluding cells of the bronchus). Median patient gene expression is calculated from bulk RNA sequencing measurements of human tumors from The Cancer Genome Atlas Program. The weighted geometric mean of normal lung cell types is calculated from the Human Protein Atlas single cell RNA sequencing atlas of normal tissue. The weighting is computed for each cell type and gene as follows: (weighted transcripts per million)=(transcripts per million)*(relative abundance of this lung cell type among all lung cells)*(NECTIN4 transcripts per million)/(this gene transcripts per million) Only surfaceome genes coding proteins of the plasma membrane which are at least partially exposed to the extracellular space are shown. AQP4 is highlighted in blue to emphasize its high gene expression in normal lung cell types and its low gene expression across most of the cancer indications displayed.

FIG. 43 shows the geometric mean of gene expression in the patient tumor indications shown in FIG. 44 including bladder, breast, esophagus, head and neck, non-small cell lung, ovary, or pancreas cancer indications compared weighted geometric mean of the gene expression of normal lung cell types with NECTIN4 gene expression greater than 10 (as shown in FIG. 18B, excluding cells of the bronchus). Median patient gene expression is calculated from bulk RNA sequencing measurements of human tumors from The Cancer Genome Atlas Program. The weighted geometric mean of normal lung cell types is calculated from the Human Protein Atlas single cell RNA sequencing atlas of normal tissue. The weighting is computed for each cell type and gene as follows: (weighted transcripts per million)=(transcripts per million)*(relative abundance of this lung cell type among all lung cells)*(NECTIN4 transcripts per million)/(this gene transcripts per million) Only surfaceome genes coding proteins of the plasma membrane which are at least partially exposed to the extracellular space are shown. AQP4 is highlighted in blue to emphasize its high gene expression in normal NECTIN4 high/DSG1 low cell types and low gene expression in cancer.

FIG. 44 shows the distribution of tumor gene expression across patients in The Cancer Genome Atlas dataset for the genes AQP4 and NECTIN4. Results are shown separately for bladder, breast, esophagus, head and neck, non-small cell lung, ovary, and pancreas cancer indications. The number of patients included for each indication is displayed under the indication name (n=number of patients). Gene expression is displayed in units of transcripts per million from bulk RNA sequencing.

FIG. 45 shows the distribution of tumor gene expression across patients in The Cancer Genome Atlas dataset for the genes AQP4, DSG1, and NECTIN4. Results are shown separately for bladder, breast, esophagus, head and neck, non-small cell lung, ovary, and pancreas cancer indications. The number of patients included for each indication is displayed under the indication name (n=number of patients). Gene expression is displayed in units of transcripts per million from bulk RNA sequencing.

FIGS. 46A-B show patient-wise co-expression of genes in patient tumors in The Cancer Genome Project of (A) AQP4 and NECTIN4 and (B) DSG1 and AQP4. Gene expression is displayed in units of transcripts per million from bulk RNA sequencing. The number of patients included for each indication is displayed under the indication name (n=number of patients).

FIG. 47 shows AQP4 and NECTIN4 gene expression in normal tissues in the Genotype-Tissue Expression project as measured by bulk RNA sequencing in units of transcripts per million. Box plots render the 25 percentile through 75 percentile of gene expression across patients with a mark displaying the median. Whiskers on the box plot are the length of 1.5 interquartile distances and outlier patients outside this range are displayed with a point. The number of individuals for each tissue are indicated (n=number of individuals). Data for lung and bladder tissue is emphasized with boxes.

FIG. 48 shows the gene expression of AQP4, DSG1, and NECTIN4 in all cell types of the (left) skin and (right) lung. Data is from the publicly available Human Protein Atlas single RNA sequencing atlas of normal tissue. The percentage of all cells in the tissue that constitute each cell type are annotated and cell types are displayed in descending order of abundance from top to bottom.

FIGS. 49A-C shows the gene expression of AQP4, DSG1, and NECTIN4 in all cell types of the (A) normal brain and (B) normal breast, and (C) all cell types from 30 normal tissues with NECTIN4 expression greater than 50 transcripts per million. Data was obtained from Human Protein Atlas single RNA sequencing atlas of normal tissue. The percentage of all cells in the tissue that constitute each cell type are annotated and cell types are displayed in descending order of abundance from top to bottom.

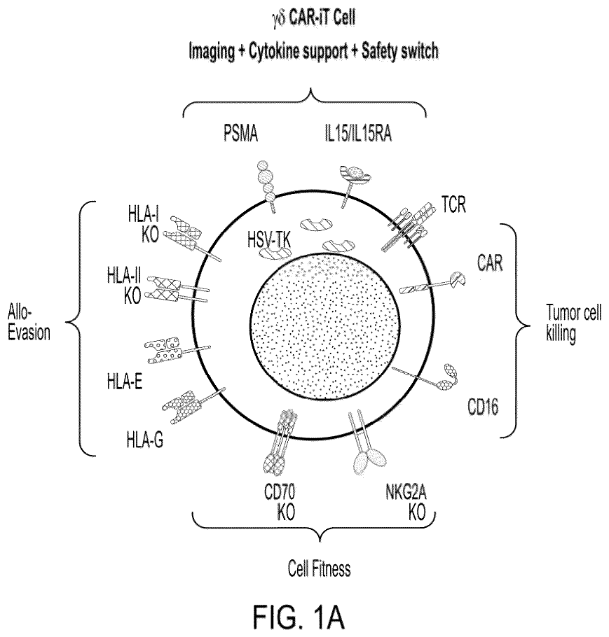

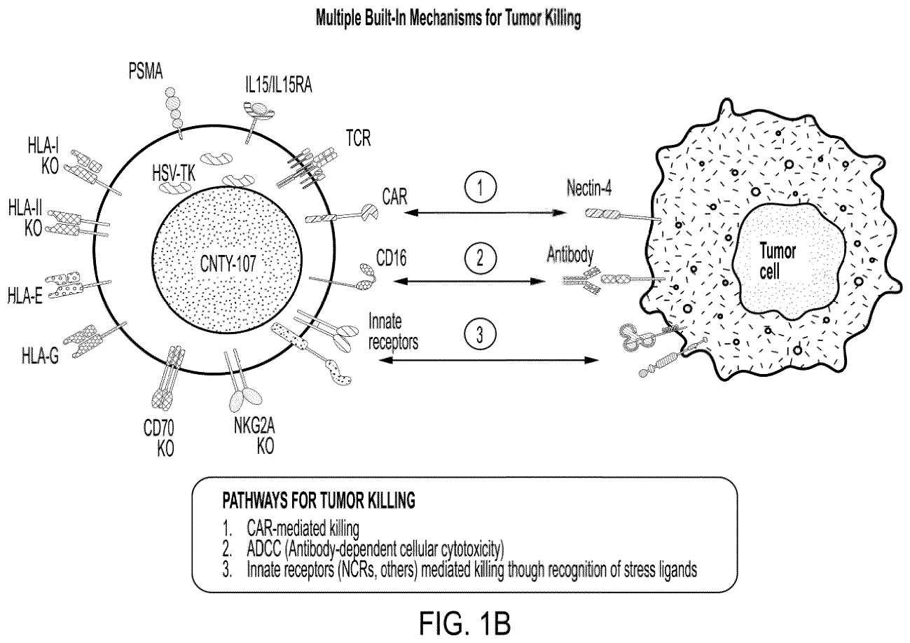

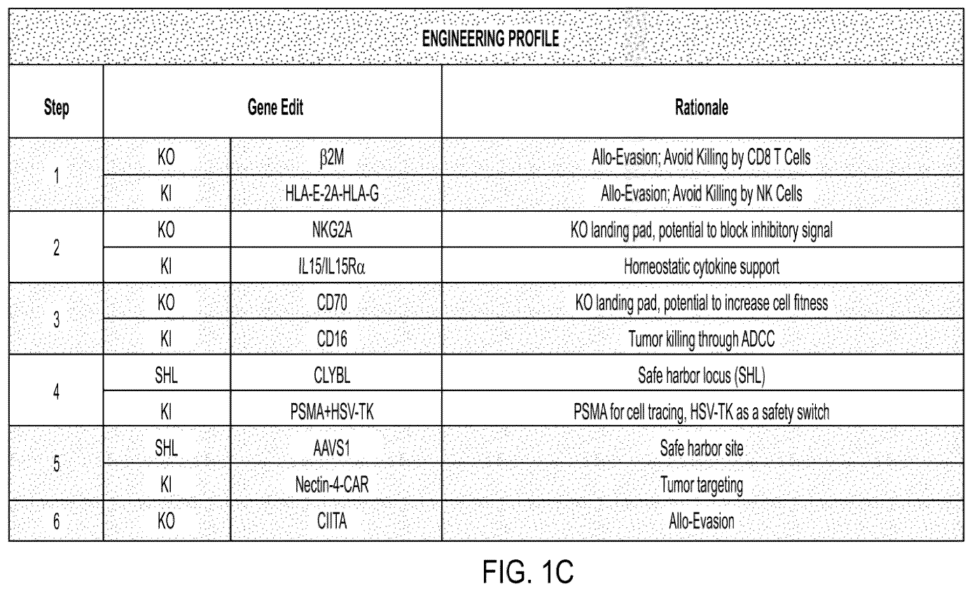

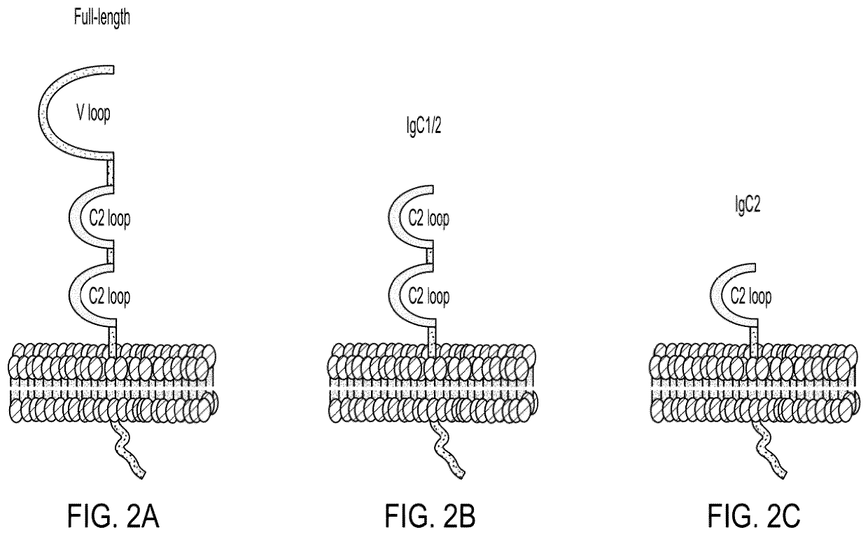

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this application pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes 10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present), and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “chimeric antigen receptor” or “CAR” refers to engineered receptors, which are grafted onto cells. In general, a CAR of the present disclosure comprises one or more extracellular domains comprising the antigen binding domain(s), one or more intracellular domains comprising one or more costimulatory and/or signaling domains, and a scaffold comprising multiple transmembrane domains and intracellular or extracellular loops, at which the one or more extracellular or intracellular domains are disposed. The antigen binding domain of the CAR targets specific antigens. The targeting regions may comprise full length heavy chain, Fab fragments, scFvs, divalent single chain antibodies or diabodies, each of which are specific to the target antigen (e.g., Nectin4 or DSG1). The antigen binding domain can be derived from the same species or a different species for or in which the CAR will be used in.

The terms “binder” or “specifically binds” or “specific for” with respect to an antigen-binding domain of a ligand like an antibody, of a fragment thereof or of a CAR refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refers to cells, preferentially T cells which are manipulated by recombinant methods well known in the art to express stably or transiently peptides or proteins which are not expressed in these cells in the natural state. For example, T cells are engineered to express an artificial construct such as a chimeric antigen receptor on their cell surface. For example, the sequences encoding the CAR may be delivered into cells using a retroviral or lentiviral vector.

The term “target” as used herein refers to an antigen or epitope associated with a cell that should be recognized specifically by an antigen binding domain, e.g. an antigen binding domain of an antibody or of a CAR. The antigen or epitope for antibody recognition can be bound to the cell surface but also be secreted, part of the extracellular membrane, or shed from the cell.

As used herein, the term “dual-targeting” refers to a protein (e.g., a chimeric protein) capable of binding to two different antigens. Specifically, a dual-targeting protein of the present disclosure (e.g., a CAR having two or more tumor or cancer antigen binding domains) does not naturally occur and is produced by a genetic engineering method or other method. In one embodiment, a primary cell, an engineered iPSC or derivative cell of the present disclosure can comprise one or more exogenous polynucleotides encoding a CAR having a first antigen binding domain that specifically binds Nectin4 and a second antigen binding domain that specifically binds DSG1. This is in contrast with other examples of the present disclosure wherein a primary cell, an engineered iPSC or derivative cell comprises one or more polynucleotides encoding a first CAR having a first antigen binding domain that specifically binds Nectin4 and a second CAR having a second antigen binding domain that specifically binds DSG1.

The terms “identical” or percent “identity,” in the context of two or more nucleic acids or polypeptide sequences (e.g., CAR polypeptides and the CAR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement)(Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” can further comprise replacement of the endogenous sequence or a nucleotide that is deleted with the one or more inserted nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell in its native form. Similarly, the term when used in reference to expression of an encoding nucleic acid refers to expression of an encoding nucleic acid natively contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof, a variant polypeptide (i.e. a mutant of the native polypeptide having less than 100% sequence identity with the native polypeptide) or fragment thereof, an engineered polypeptide or peptide fragment, a therapeutic peptide or polypeptide, an imaging marker, a selectable marker, and the like.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “engineered immune cell” refers to an immune cell, also referred to as an immune effector cell, that has been genetically modified by the addition of exogenous genetic material in the form of DNA or RNA to the total genetic material of the cell.

### Induced Pluripotent Stem Cells (IPSCs) And Immune Effector Cells

IPSCs have unlimited self-renewing capacity. Use of iPSCs enables cellular engineering to produce a controlled cell bank of modified cells that can be expanded and differentiated into desired immune effector cells, supplying large amounts of homogeneous allogeneic therapeutic products.

Provided herein are genetically engineered IPSCs and derivative cells thereof. The selected genomic modifications provided herein enhance the therapeutic properties of the derivative cells. The derivative cells are functionally improved and suitable for allogenic off-the-shelf cell therapies following a combination of selective modalities being introduced to the cells at the level of iPSC through genomic engineering. This approach can help to reduce the side effects mediated by CRS/GVHD and prevent long-term autoimmunity while providing excellent efficacy.

As used herein, the term “differentiation” is the process by which an unspecialized (“uncommitted”) or less specialized cell acquires the features of a specialized cell. Specialized cells include, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma or the embryo proper. For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the terms “reprogramming” or “dedifferentiation” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “induced pluripotent stem cells” or, iPSCs, means that the stem cells are produced from differentiated adult, neonatal or fetal cells that have been induced or changed or reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” or “HPCs” refers to cells which are committed to a hematopoietic lineage but are capable of further hematopoietic differentiation. Hematopoietic stem cells include, for example, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). As used herein, “CD34+ hematopoietic progenitor cell” refers to an HPC that expresses CD34 on its surface.

As used herein, the term “immune cell” or “immune effector cell” refers to a cell that is involved in an immune response. Immune response includes, for example, the promotion of an immune effector response. Examples of immune cells include T cells, B cells, natural killer (NK) cells, mast cells, and myeloid-derived phagocytes.

As used herein, the terms “T lymphocyte” and “T cell” are used interchangeably and refer to a type of white blood cell that completes maturation in the thymus and that has various roles in the immune system. A T cell can have the roles including, e.g., the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupTl, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (gd T cells), and the like. Additional types of helper T cells include cells such as Th3 (Treg), Th17, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refers to a subset of peripheral blood lymphocytes defined by the expression of CD56 and CD45 and the absence of the T cell receptor (TCR chains). The NK cell can also refer to a genetically engineered NK cell, such as a NK cell modified to express a chimeric antigen receptor (CAR). The NK cell can also be differentiated from a stem cell or progenitor cell.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK or “iNK” cells or derivative T or “iT” cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing.

The induced pluripotent stem cell (iPSC) parental cell lines may be generated from peripheral blood mononuclear cells (PBMCs) or T-cells using any known method for introducing re-programming factors into non-pluripotent cells such as the episomal plasmid-based process as previously described in U.S. Pat. Nos. 8,546,140; 9,644,184; 9,328,332; and 8,765,470, the complete disclosures of which are incorporated herein by reference. The reprogramming factors may be in a form of polynucleotides, and thus are introduced to the non-pluripotent cells by vectors such as a retrovirus, a Sendai virus, an adenovirus, an episome, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the iPSC's are clonal iPSC's or are obtained from a pool of iPSCs and the genome edits are introduced by making one or more targeted integration and/or in/del at one or more selected sites. In another embodiment, the iPSC's are obtained from human T cells having antigen specificity and a reconstituted TCR gene (hereinafter, also refer to as “T-iPS” cells) as described in U.S. Pat. Nos. 9,206,394, and 10,787,642 hereby incorporated by reference into the present application.

According to a particular aspect, the application relates to an induced pluripotent stem cell (iPSC) cell or a derivative cell thereof comprising: (i) an exogenous polynucleotide encoding a chimeric antigen receptor (CAR); (ii) an exogenous polynucleotide encoding a truncated epithelial growth factor (tEGFR) variant and an interleukin 15 (IL-15), wherein the tEGFR variant and IL-15 are operably linked by an autoprotease peptide sequence, such as the porcine teschovirus-1 2A (P2A); and (iii) a deletion or reduced expression of B2M and CIITA genes.

### I. Chimeric Antigen Receptor (CAR) Expression

According to embodiments of the application, an iPSC or a derivative cell thereof comprises one or more exogenous polynucleotides encoding a chimeric antigen receptor (CAR), wherein the CAR targets a Nectin4 antigen. Nectins are cell adhesion molecules (CAMs) involved in Ca²⁺-independent cell-cell interactions. The Nectin family includes four Nectins:
Nectins 1-3 are enriched in normal adult tissues
Nectin4 is mostly expressed during fetal development and its expression declines in adult tissues (low expression levels in skin, bladder, placenta, oral mucosa, and tonsils).

Nectins interact with other cell surface molecules including cadherins, integrins and growth factor receptors. These interactions help modulate cell adhesion, migration and proliferation. Nectin4 dimers bind to Nectin-1 or Nectin4 on adjacent cells. Nectin4 also binds TIGIT on immune cells and this interaction leads to inhibition of NK cells.

Accordingly, Nectin4 is a suitable target for a CAR of the invention because it is expressed in high frequency in bladder, breast, lung, pancreatic, ovarian, head & neck, and esophageal cancers. The highest levels of expression of Nectin4 are seen in bladder, breast, lung and pancreatic cancers. Clinical validation of Nectin4 as a tumor target has been demonstrated by the approval of Enfortumab vedotin for the treatment of urothelial cancer

Thus in one embodiment, the CAR targets a Nectin4 antigen and the targeting region (e.g., the extracellular domain) of the CAR comprises an antibody fragment (e.g., a VHH domain). In other embodiments, an iPSC or a derivative cell thereof comprises one or more first exogenous polynucleotides encoding a single CAR targeting a Nectin4 antigen.

In some embodiments, an iPSC or a derivative cell thereof comprises one or more first exogenous polynucleotides encoding a CAR (e.g., targeting Nectin4) and an additional CAR targeting another antigen. In some embodiments, the antigen targeted by the additional CAR is selected from the group consisting of CD70, Folate Receptor alpha, FSHR, mesothelin, and SLITRK6. In other embodiments, an iPSC or a derivative cell thereof comprises one or more first exogenous polynucleotides encoding a dual-targeting CAR targeting a Nectin4 antigen and an another antigen selected from the group consisting of CD70, Folate Receptor alpha, FSHR, mesothelin, and SLITRK6. Each of the binding domains of any of the CAR, the additional CAR, or the dual-targeting CAR can be, for example, independently selected from an scFv and a VHH.

As used herein, the term “chimeric antigen receptor” (CAR) refers to a recombinant polypeptide comprising at least an extracellular domain that binds specifically to an antigen or a target, a transmembrane domain and an intracellular signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen-binding domain,” “extracellular domain,” or “extracellular ligand binding domain” refers to the part of a CAR that is located outside of the cell membrane and is capable of binding to an antigen, target or ligand.

As used herein, the term “hinge region” or “hinge domain” refers to the part of a CAR that connects two adjacent domains of the CAR protein, i.e., the extracellular domain and the transmembrane domain of the CAR protein.

As used herein, the term “transmembrane domain” refers to the portion of a CAR that extends across the cell membrane and anchors the CAR to cell membrane.

As used herein, the term “intracellular signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by an immune cell (e.g., NK cell or T cell) that provides the primary cytoplasmic signaling sequence(s) that regulate primary activation of receptors in a stimulatory way for at least some aspect of the immune cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain embodiments, the extracellular domain comprises an antigen-binding domain and/or an antigen-binding fragment. The antigen-binding fragment can, for example, be an antibody or antigen-binding fragment thereof that specifically binds a tumor antigen. The antigen-binding fragments of the application possess one or more desirable functional properties, including but not limited to high-affinity binding to a tumor antigen, high specificity to a tumor antigen, the ability to stimulate complement-dependent cytotoxicity (CDC), antibody-dependent phagocytosis (ADPC), and/or antibody-dependent cellular-mediated cytotoxicity (ADCC) against cells expressing a tumor antigen, and the ability to inhibit tumor growth in subjects in need thereof and in animal models when administered alone or in combination with other anti-cancer therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the application can be of any of the five major classes or corresponding sub-classes. Preferably, the antibodies of the application are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the application can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (VHH) of a camelid antibody, or any other antibody fragment that binds to an antigen but does not comprise a complete antibody structure. An antigen-binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, a bispecific antibody comprises a V_{H}H having binding specificity for a first epitope, and a V_{H}H having binding specificity for a second epitope. In an embodiment, the term X/Y loop (wherein ‘X’ and ‘Y’ are antigens such as Nectin4 and an antigen selected from the group consisting of CD70, Folate Receptor alpha, FSHR, mesothelin, and SLITRK6 refers to an extracellular region in which one scFv is nested in between the VL and VH of the other scFv. In some embodiments, X and Y may be the same antigen. In some embodiments, X and Y may be different antigens. In some embodiments, X and Y are tumor antigens.

As used herein, an antigen-binding domain or antigen-binding fragment that “specifically binds to a tumor antigen” refers to an antigen-binding domain or antigen-binding fragment that binds a tumor antigen, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen-binding domain or antigen-binding fragment can be determined by using surface plasmon resonance, such as by using a biosensor system, e.g., a Biacore® system, or by using bio-layer interferometry technology, such as an Octet RED96 system.

The smaller the value of the KD of an antigen-binding domain or antigen-binding fragment, the higher affinity that the antigen-binding domain or antigen-binding fragment binds to a target antigen.

In various embodiments, antibodies or antibody fragments suitable for use in the CAR of the present disclosure include, but are not limited to, monoclonal antibodies, bispecific antibodies, multispecific antibodies, chimeric antibodies, polypeptide-Fc fusions, single-chain Fvs (scFv), single chain antibodies, Fab fragments, F(ab′) fragments, disulfide-linked Fvs (sdFv), masked antibodies (e.g., Probodies®), Small Modular ImmunoPharmaceuticals (“SMIPs™”), intrabodies, minibodies, single domain antibody variable domains, nanobodies, VHHs, diabodies, tandem diabodies (TandAb®), anti-idiotypic (anti-Id) antibodies (including, e.g., anti-Id antibodies to antigen-specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding fragment is an Fab fragment, an Fab′ fragment, an F(ab′)2 fragment, an scFv fragment, an Fv fragment, a dsFv diabody, a VHH, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody. In some embodiments, the antigen-binding fragment is an scFv fragment. In some embodiments, the antigen-binding fragment is a VHH.

In some embodiments, at least one of the extracellular tag-binding domain, the antigen-binding domain, or the tag comprises a single-domain antibody or nanobody. In some embodiments, at least one of the extracellular tag-binding domain, the antigen-binding domain, or the tag comprises a VHH.

In some embodiments, the extracellular tag-binding domain and the tag each comprise a VHH.

In some embodiments, the extracellular tag-binding domain, the tag, and the antigen-binding domain each comprise a VHH.

In some embodiments, at least one of the extracellular tag-binding domain, the antigen-binding domain, or the tag comprises an scFv.

In some embodiments, the extracellular tag-binding domain and the tag each comprise an scFv.

In some embodiments, the extracellular tag-binding domain, the tag, and the antigen-binding domain each comprise a scFv.

Alternative scaffolds to immunoglobulin domains that exhibit similar functional characteristics, such as high-affinity and specific binding of target biomolecules, may also be used in the CARs of the present disclosure. Such scaffolds have been shown to yield molecules with improved characteristics, such as greater stability or reduced immunogenicity. Non-limiting examples of alternative scaffolds that may be used in the CAR of the present disclosure include engineered, tenascin-derived, tenascin type III domain (e.g., Centyrin™); engineered, gamma-B crystallin-derived scaffold or engineered, ubiquitin-derived scaffold (e.g., Affilins); engineered, fibronectin-derived, 10th fibronectin type III (10Fn3) domain (e.g., monobodies, AdNectins™, or AdNexins™); engineered, ankyrin repeat motif containing polypeptide (e.g., DARPins™); engineered, low-density-lipoprotein-receptor-derived, A domain (LDLR-A) (e.g., Avimers™); lipocalin (e.g., anticalins); engineered, protease inhibitor-derived, Kunitz domain (e.g., EETI-II/AGRP, BPTI/LACI-D1/ITI-D2), engineered, Protein-A-derived, Z domain (Affibodies™); Sac7d-derived polypeptides (e.g., Nanoffitins® or affitins); engineered, Fyn-derived, SH2 domain (e.g., Fynomers®); CTLD₃ (e.g., Tetranectin); thioredoxin (e.g., peptide aptamer); KALBITOR®, the β-sandwich (e.g., iMab); miniproteins; C-type lectin-like domain scaffolds; engineered antibody mimics; and any genetically manipulated counterparts of the foregoing that retains its binding functionality (Worn A, Pluckthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biotechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Skerra, Current Opin. in Biotech., 2007 18: 295-304; Byla P et al., J Biol Chem 285: 12096 (2010); Zoller F et al., Molecules 16: 2467-85 (2011), each of which is incorporated by reference in its entirety).

In some embodiments, the alternative scaffold is Affilin or Centyrin.

In some embodiments, the first polypeptide of the CARs of the present disclosure comprises a leader sequence. The leader sequence may be positioned at the N-terminus the extracellular tag-binding domain. The leader sequence may be optionally cleaved from the extracellular tag-binding domain during cellular processing and localization of the CAR to the cellular membrane. Any of various leader sequences known to one of skill in the art may be used as the leader sequence. Non-limiting examples of peptides from which the leader sequence may be derived include granulocyte-macrophage colony-stimulating factor receptor (GMCSFR), FcεR, human immunoglobulin (IgG) heavy chain (HC) variable region, CD8α, or any of various other proteins secreted by T cells. In various embodiments, the leader sequence is compatible with the secretory pathway of a T cell. In certain embodiments, the leader sequence is derived from human immunoglobulin heavy chain (HC).

In some embodiments, the leader sequence is derived from GMCSFR. In one embodiment, the GMCSFR leader sequence comprises the amino acid sequence set forth in SEQ ID NO: 1, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1.

In some embodiments, the first polypeptide of the CARs of the present disclosure comprise a transmembrane domain, fused in frame between the extracellular tag-binding domain and the cytoplasmic domain.

The transmembrane domain may be derived from the protein contributing to the extracellular tag-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane region(s) of) the α, β or ζ chain of the T-cell receptor (TCR), CD28, CD3 epsilon, CD45, CD4, CD5, CD8, CD8α, CD9, CD16, CD22, CD28, CD33, CD37, CD40, CD64, CD80, CD86, CD134, CD137, or CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FecεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases, it will be desirable to employ the transmembrane domain of ζ, η or FcεR1γ and −β, MB1 (Igα), B29 or CD3-γ, ζ, or η, in order to retain physical association with other members of the receptor complex.

In some embodiments, the transmembrane domain is derived from CD8 or CD28. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23. In one embodiment, the CD28 transmembrane domain comprises the amino acid sequence set forth in SEQ ID NO: 24, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 24.

In some embodiments, the first polypeptide of the CAR of the present disclosure comprises a spacer region between the extracellular tag-binding domain and the transmembrane domain, wherein the tag-binding domain, linker, and the transmembrane domain are in frame with each other.

The term “spacer region” as used herein generally means any oligo- or polypeptide that functions to link the tag-binding domain to the transmembrane domain. A spacer region can be used to provide more flexibility and accessibility for the tag-binding domain. A spacer region may comprise up to 300 amino acids, preferably 10 to 100 amino acids and most preferably 25 to 50 amino acids. A spacer region may be derived from all or part of naturally occurring molecules, such as from all or part of the extracellular region of CD8, CD4 or CD28, or from all or part of an antibody constant region. Alternatively, the spacer region may be a synthetic sequence that corresponds to a naturally occurring spacer region sequence, or may be an entirely synthetic spacer region sequence. Non-limiting examples of spacer regions which may be used in accordance to the disclosure include a part of human CD8α chain, partial extracellular domain of CD28, FcγRllla receptor, IgG, IgM, IgA, IgD, IgE, an Ig hinge, or functional fragment thereof. In some embodiments, additional linking amino acids are added to the spacer region to ensure that the antigen-binding domain is an optimal distance from the transmembrane domain. In some embodiments, when the spacer is derived from an Ig, the spacer may be mutated to prevent Fc receptor binding.

In some embodiments, the spacer region comprises a hinge domain. The hinge domain may be derived from CD8, CD8α, CD28, or an immunoglobulin (IgG). For example, the IgG hinge may be from IgG1, IgG2, IgG3, IgG4, IgG4 CH3, IgM1, IgM2, IgA1, IgA2, IgD, IgE, or a chimera thereof.

In certain embodiments, the hinge domain comprises an immunoglobulin IgG hinge or functional fragment thereof. In certain embodiments, the IgG hinge is from IgG1, IgG2, IgG3, IgG4, IgG4 CH3, IgM1, IgM2, IgA1, IgA2, IgD, IgE, or a chimera thereof. In certain embodiments, the hinge domain comprises the CH1, CH2, CH3 and/or hinge region of the immunoglobulin. In certain embodiments, the hinge domain comprises the core hinge region of the immunoglobulin. The term “core hinge” can be used interchangeably with the term “short hinge” (a.k.a “SH”). Non-limiting examples of suitable hinge domains are the core immunoglobulin hinge regions include EPKSCDKTHTCPPCP (SEQ ID NO: 57) from IgG1, ERKCCVECPPCP (SEQ ID NO: 58) from IgG2, ELKTPLGDTTHTCPRCP(EPKSCDTPPPCPRCP)₃ (SEQ ID NO: 59) from IgG3, ESKYGPPCPSCP (SEQ ID NO: 60) from IgG4 (see also Wypych et al., JBC 2008 283(23): 16194-16205, which is incorporated herein by reference in its entirety for all purposes), and ESKYGPPCPPCPGQPREPQVYTLPPSQEEMTKNQVSLTCLVKGFYPSDIAVEWES NGQPENNYKTTPPVLDSDGSFFLYSRLTVDKSRWQEGNVFSCSVMHEALHNHY TQKSLSLSLGK (SEQ ID NO: 96), or a variant thereof having at least 50, at least 55, at least 60, at least 65, at least 70, at least 75, at least 80, at least 85, at least 90, at least 95, at least 96, at least 97, at least 98 or at least 99%, sequence identity. In certain embodiments, the hinge domain is a fragment of the immunoglobulin hinge.

In some embodiments, the hinge domain is derived from CD8 or CD28. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21. In one embodiment, the CD28 hinge domain comprises the amino acid sequence set forth in SEQ ID NO: 22, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 22.

In some embodiments, the transmembrane domain and/or hinge domain is derived from CD8 or CD28. In some embodiments, both the transmembrane domain and hinge domain are derived from CD8. In some embodiments, both the transmembrane domain and hinge domain are derived from CD28.

In certain aspects, the first polypeptide of CARs of the present disclosure comprise a cytoplasmic domain, which comprises at least one intracellular signaling domain. In some embodiments, cytoplasmic domain also comprises one or more co-stimulatory signaling domains.

The cytoplasmic domain is responsible for activation of at least one of the normal effector functions of the host cell (e.g., T cell) in which the CAR has been placed in. The term “effector function” refers to a specialized function of a cell. Effector function of a T-cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3ε, CD3γ, CD3ζ, CD5, CD22, CD226, CD66d, CD79a, and CD79b.

In some embodiments, the cytoplasmic domain comprises a CD3ζ signaling domain. In one embodiment, the CD3ζ signaling domain comprises the amino acid sequence set forth in SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6.

In some embodiments, the cytoplasmic domain further comprises one or more co-stimulatory signaling domains. In some embodiments, the one or more co-stimulatory signaling domains are derived from CD28, 41BB, IL2Rb, CD40, OX40 (CD134), CD80, CD86, CD27, ICOS, NKG2D, DAP10, DAP12, 2B4 (CD244), BTLA, CD30, GITR, CD226, CD79A, and HVEM.

In one embodiment, the co-stimulatory signaling domain is derived from 41BB. In one embodiment, the 41BB co-stimulatory signaling domain comprises the amino acid sequence set forth in SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8.

In one embodiment, the co-stimulatory signaling domain is derived from IL2Rb. In one embodiment, the IL2Rb co-stimulatory signaling domain comprises the amino acid sequence set forth in SEQ ID NO: 9, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9.

In one embodiment, the co-stimulatory signaling domain is derived from CD40. In one embodiment, the CD40 co-stimulatory signaling domain comprises the amino acid sequence set forth in SEQ ID NO: 10, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10.

In one embodiment, the co-stimulatory signaling domain is derived from OX40. In one embodiment, the OX40 co-stimulatory signaling domain comprises the amino acid sequence set forth in SEQ ID NO: 11, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 11.

In one embodiment, the co-stimulatory signaling domain is derived from CD80. In one embodiment, the CD80 co-stimulatory signaling domain comprises the amino acid sequence set forth in SEQ ID NO: 12, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12.

In one embodiment, the co-stimulatory signaling domain is derived from CD86. In one embodiment, the CD86 co-stimulatory signaling domain comprises the amino acid sequence set forth in SEQ ID NO: 13, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 13.

In one embodiment, the co-stimulatory signaling domain is derived from CD27. In one embodiment, the CD27 co-stimulatory signaling domain comprises the amino acid sequence set forth in SEQ ID NO: 14, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 14.

In one embodiment, the co-stimulatory signaling domain is derived from ICOS. In one embodiment, the ICOS co-stimulatory signaling domain comprises the amino acid sequence set forth in SEQ ID NO: 15, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15.

In one embodiment, the co-stimulatory signaling domain is derived from NKG2D. In one embodiment, the NKG2D co-stimulatory signaling domain comprises the amino acid sequence set forth in SEQ ID NO: 16, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 16.

In one embodiment, the co-stimulatory signaling domain is derived from DAP10. In one embodiment, the DAP10 co-stimulatory signaling domain comprises the amino acid sequence set forth in SEQ ID NO: 17, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 17.

In one embodiment, the co-stimulatory signaling domain is derived from DAP12. In one embodiment, the DAP12 co-stimulatory signaling domain comprises the amino acid sequence set forth in SEQ ID NO: 18, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 18.

In one embodiment, the co-stimulatory signaling domain is derived from 2B4 (CD244). In one embodiment, the 2B4 (CD244) co-stimulatory signaling domain comprises the amino acid sequence set forth in SEQ ID NO: 19, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 19.

In some embodiments, the CAR of the present disclosure comprises one costimulatory signaling domains. In some embodiments, the CAR of the present disclosure comprises two or more costimulatory signaling domains. In certain embodiments, the CAR of the present disclosure comprises two, three, four, five, six or more costimulatory signaling domains.

In some embodiments, the signaling domain(s) and costimulatory signaling domain(s) can be placed in any order. In some embodiments, the signaling domain is upstream of the costimulatory signaling domains. In some embodiments, the signaling domain is downstream from the costimulatory signaling domains. In the cases where two or more costimulatory domains are included, the order of the costimulatory signaling domains could be switched.

Non-limiting exemplary CAR regions and sequences are provided in Table 1, including amino acid and nucleic acid sequences for the various CAR constructs of the present disclosure.

TABLE 1

| | | | |
|---|---|---|---|
| | | UniProt | SEQ ID |
| CAR regions | Sequence | Id | NO |

Nectin4 Binding Domains:

| | | |
|---|---|---|
| DB01_C01 | EVQLLESGGGLVQAGGSLRLSCAASGSF | 105 |
| P2112 | FSIYAMGWFRQAPGKEREFVAAYISSGG | |
| (amino acid) | LTSYADSVKGRFTISRDNAKNTVYLQMN | |
| | SLKPEDTAVYYCAADLGAQTGYVQYDY | |
| | WGQGTQVTVSS | |
| DB0_B01 | EVQLLESGGGLVQPGGSLRLSCAASGFVS | 106 |
| P2106 | SIYFMGWFRQAPGKEREFVSSSIGKGGST | |
| (amino acid) | RYADSVKGRFTISRDNSKNTLYLQMNSL | |
| | KPEDTAVYYCAGDEGLGTAHAEYDYWG | |
| | QGTQVTVSS | |
| DB01_B10 | EVQLLESGGGLVQAGGSLRLSCAASGGIS | 107 |
| P2110 | EFYFMGWFRQAPGKEREFVAAEISPGSY | |
| (amino acid) | TNYADSVKGRFTISRDNAKNTVYLQMNS | |
| | LKPEDTAVYYCAADRDGDTYTAEYDYW | |
| | GQGTQVTVSS | |
| DB01_F03 | EVQLLESGGGLVQPGGSLRLSCAASGSIS | 108 |
| P2121 | SFYYIGWFRQAPGKEREFVSSRITSGGST | |
| (amino acid) | YYRDSVKGRFTISRDNSKNTLYLQMNSL | |
| | KPEDTAVYYCAAGTSRDYYYWGQGTQ | |
| | VTVSS | |
| DB01_A11 | EVQLLESGGGLVQPGGSLRLSCAASGST | 109 |
| P2105 | SSIGIMGWFRQAPGKERELVSSITAGGST | |
| (amino acid) | YYADSVKGRFTISRDNSKNTLYLQMNSL | |
| | KPEDTAVYYCNAHVGYGRVHDVDYWG | |
| | QGTQVTVSS | |
| DB01_E03 | EVQLLESGGGLVQPGGSLRLSCAASGFV | 110 |
| P2120 | SPSYIMGWFRQAPGKEREFVSSVIEYRGS | |
| (amino acid) | TYYLDSVKGRFTISRDNSKNTLYLQMNS | |
| | LKPEDTAVYYCAAGTPGGYDYWGQGT | |
| | QVTVSS | |
| NEC_S_2 | EVQLLESGGGLVQPGGSLRLSCAASGST | 111 |
| (amino acid) | FSSNAMGWYRQAPGKERELVSSISGSGG | |
| | STRYADSVKGRFTISRDNSKNTLYLQMN | |
| | SLKPEDTAVYYCASYVLYLREYWGQGT | |
| | QVTVSS | |
| NEC_S_5 | EVQLLESGGGLVQPGGSLRLSCAASGLT | 112 |
| (amino acid) | FRYNAMGWYRQAPGKEREFVSAISGSG | |
| | GGTYYADSVKGRFTISRDNSKNTLYLQM | |
| | NSLKPEDTAVYYCAAEGLYDYWGQGTQ | |
| | VTVSS | |
| NEC_S_11 | EVQLLESGGGLVQPGGSLRLSCAASGLT | 113 |
| P3112 | SSGYAMGWYRQAPGKERELVSSISSSGG | |
| (amino acid) | LTHYADSVKGRFTISRDNSKNTLYLQMN | |
| | SLKPEDTAVYYCDADIAYTGADYWGQG | |
| | TQVTVSS | |
| NEC_S_16 | EVQLLESGGGLVQPGGSLRLSCAASGFT | 114 |
| (amino acid) | FSDYYMGWYRQAPGKEREFVSAISSTGG | |
| | SPYYADSVKGRFTISRDNSKNTLYLQMN | |
| | SLKPEDTAVYYCAVEVPYDYWGQGTQV | |
| | TVSS | |
| NEC_S_31 | EVQLLESGGGLVQPGGSLRLSCAASGFT | 115 |
| P3113 | YSSYAMGWYRQAPGKERELVSSISGSGG | |
| (amino acid) | STRYADSVKGRFTISRDNSKNTLYLQMN | |
| | SLKPEDTAVYYCAVAIGVGDYWGQGTQ | |
| | VTVSS | |
| NEC_S_55 | EVQLLESGGGLVQPGGSLRLSCAASGFT | 116 |
| P3114 | LSSYAMGWYRQAPGKERELVSSISGSGG | |
| (amino acid) | STRYADSVKGRFTISRDNSKNTLYLQMN | |
| | SLKPEDTAVYYCAAYIGGDYLGQGTQV | |
| | TVSS | |
| NEC_S_56 | EVQLLESGGGLVQPGGSLRLSCAASGLT | 117 |
| (amino acid) | LSSYAMGWFRQAPGKERELVSSISGSGG | |
| | STRYADSVKGRFTISRDNSKNTLYLQMN | |
| | SLKPEDTAVYYCAVDIRLTDYWGQGTQ | |
| | VTVSS | |
| NEC_S_64 | EVQLLESGGGLVQPGGSLRLSCAASGFA | 118 |
| (amino acid) | YSSYYMGWYRQAPGKEREFVSAISSSGG | |
| | GTYYADSVKGRFTISRDNSKNTLYLQMN | |
| | SLKPEDTAVYYCAVELTYNYWGQGTQV | |
| | TVSS | |
| NEC_S_81 | EVQLLESGGGLVQPGGSLRLSCAASGFT | 119 |
| (amino acid) | FRSYAMGWFRQAPGKEREGVSVITGTG | |
| | GSTYYADSVKGRFTISRDNSKNTLYLQM | |
| | NSLKPEDTAVYYCARAYLSGYLYEYWG | |
| | QGTQVTVSS | |
| NEC_S_85 | EVQLLESGGGLVQPGGSLRLSCAASGLT | 120 |
| (amino acid) | LSSYAMGWFRQAPGKERELVSSISGSGG | |
| | STRYADSVKGRFTISRDNSKNTLYLQMN | |
| | SLKPEDTAVYYCAVDIRLTDYWGQGTQ | |
| | VTVSS | |
| NEC_M_5 | EVQLLESGGGLVQPGGSLRLSCAASGLS | 121 |
| P3106 | FSSYSMGWYRQAPGKEREFVSAISGSSG | |
| (amino acid) | STNYADSVKGRFTISRDNSKNTLYLQMN | |
| | SLKPEDTAVYYCAAEHRVTTSGVFYDY | |
| | WGQGTQVTVSS | |
| NEC_M_8 | EVQLLESGGGLVQPGGSLRLSCAASGFT | 122 |
| P3107 | FSSYAMGWYRQAPGKEREFVSSISGSGG | |
| (amino acid) | LTRYADSVKGRFTISRDNSKNTLYLQMN | |
| | SLKPEDTAVYYCAVTTGYQGGVYDYW | |
| | GQGTQVTVSS | |
| NEC_M_17 | EVQLLESGGGLVQPGGSLRLSCAASGFT | 123 |
| P3108 | YSGYAMGWYRQAPGKERELVSSISGSGT | |
| (amino acid) | LTSYADSVKGRFTISRDNSKNTLYLQMN | |
| | SLKPEDTAVYYCDVDIPVGDATTVGDY | |
| | WGQGTQVTVSS | |
| NEC_M_21 | EVQLLESGGGLVQPGGSLRLSCAASGLT | 124 |
| (amino acid) | FSSNAMGWYRQAPGKERELVSSISGSGG | |
| | STNYADSVKGRFTISRDNSKNTLYLQMN | |
| | SLKPEDTAVYYCAAEIATYYDFADYWG | |
| | QGTQVTVSS | |
| NEC_M_29 | EVQLLESGGGLVQPGGSLRLSCAASGFT | 125 |
| (amino acid) | YTSYVMGWYRQAPGKERELVSAIYGSA | |
| | GSGYYADSVKGRFTISRDNSKNTLYLQM | |
| | NSLKPEDTAVYYCARVTTTTHLWSAEY | |
| | WGQGTQVTVSS | |
| NEC_M_44 | EVQLLESGGGLVQPGGSLRLSCAASGRT | 126 |
| P3109 | LSSYAMGWYRQAPGKERELVSSISGSGG | |
| (amino acid) | STRYADSVKGRFTISRDNSKNTLYLQMN | |
| | SLKPEDTAVYYCAVYILELAPGAEYWGQ | |
| | GTQVTVSS | |
| NEC_M_46 | EVQLLESGGGLVQPGGSLRLSCAASGYT | 127 |
| P3110 | FSDYAMGWYRQAPGKERELVSSISGSGG | |
| (amino acid) | STRYADSVKGRFTISRDNSKNTLYLQMN | |
| | SLKPEDTAVYYCAAVIRQPSTGFYEYWG | |
| | QGTQVTVSS | |
| NEC_M_66 | EVQLLESGGGLVQPGGSLRLSCAASGFA | 128 |
| (amino acid) | YSSYSIGWYRQAPGKERELVSAISGSGGS | |
| | SRYADSVKGRFTISRDNSKNTLYLQMNS | |
| | LKPEDTAVYYCAAIAYVYTSTARNYWG | |
| | QGTQVTVSS | |
| NEC_M_82 | EVQLLESGGGLVQPGGSLRLSCAASGFT | 129 |
| (amino acid) | YSDYAMGWYRQAPGKERELVSAISSYG | |
| | GVTNYADSVKGRFTISRDNSKNTLYLQM | |
| | NSLKPEDTAVYYCAAYGALSIRGYDYW | |
| | GQGTQVTVSS | |
| NEC_M_84 | EVQLLESGGGLVQPGGSLRLSCAASGFA | 130 |
| (amino acid) | FSIDAMGWYRQAPGKEREGVSSISGSGG | |
| | LTRYADSVKGRFTISRDNSKNTLYLQMN | |
| | SLKPEDTAVYYCDTAVLTGYSEAFDYW | |
| | GQGTQVTVSS | |
| DB01_C01 | GAGGTACAACTTTTGGAGTCAGGCGGC | 131 |
| P2112 | GGGTTGGTCCAGGCGGGTGGCTCACTC | |
| (nucleotide) | CGCCTTAGTTGTGCCGCCTCAGGGTCA | |
| | TTCTTTAGTATCTACGCTATGGGCTGGT | |
| | TTCGACAGGCCCCTGGTAAGGAACGTG | |
| | AGTTTGTGGCCGCCTACATTTCCTCAG | |
| | GGGGGCTCACCAGCTACGCGGATAGTG | |
| | TTAAGGGTAGATTCACCATCTCCAGAG | |
| | ACAATGCAAAGAATACGGTATACCTCC | |
| | AAATGAACAGCCTGAAGCCTGAAGAC | |
| | ACGGCTGTCTACTATTGCGCAGCAGAC | |
| | TTGGGAGCCCAGACCGGATACGTTCAG | |
| | TACGACTACTGGGGGCAGGGAACCCA | |
| | GGTGACGGTCTCGAGC | |
| DB01_B01 | GAGGTACAACTTTTGGAGTCAGGCGGC | 132 |
| P2106 | GGCCTGGTGCAGCCCGGAGGGAGTCTC | |
| (nucleotide) | CGACTGTCTTGCGCTGCATCTGGATTC | |
| | GTGAGCTCTATATACTTTATGGGATGG | |
| | TTCAGGCAGGCTCCTGGGAAGGAGCGC | |
| | GAGTTTGTGTCTAGTAGTATTGGCAAG | |
| | GGTGGCTCAACACGCTATGCGGATTCT | |
| | GTGAAAGGGAGGTTCACAATAAGCAG | |
| | GGACAACTCAAAGAATACACTGTACCT | |
| | CCAGATGAACTCCTTAAAACCAGAGGA | |
| | TACTGCAGTCTATTACTGTGCTGGAGA | |
| | TGAGGGATTGGGAACTGCACATGCTGA | |
| | ATACGACTACTGGGGCCAGGGGACCCA | |
| | GGTGACGGTCTCGAGC | |
| DB01_B10 | GAGGTACAACTTTTGGAGTCAGGCGGA | 133 |
| P2110 | GGACTCGTGCAGGCTGGAGGGTCCCTT | |
| (nucleotide) | AGGCTCAGCTGCGCCGCTTCCGGAGGG | |
| | ATCTCCGAATTCTATTTCATGGGGTGGT | |
| | TTAGACAAGCGCCCGGAAAGGAAAGG | |
| | GAATTCGTCGCAGCAGAGATCTCACCT | |
| | GGAAGCTACACCAACTACGCAGATAGC | |
| | GTGAAGGGGCGCTTTACCATCTCTAGG | |
| | GATAACGCGAAAAATACAGTCTACCTC | |
| | CAAATGAACTCTTTGAAGCCAGAGGAT | |
| | ACCGCCGTGTATTATTGCGCTGCCGAC | |
| | AGGGACGGAGACACATATACAGCAGA | |
| | ATATGATTATTGGGGCCAGGGGACCCA | |
| | GGTGACGGTCTCGAGC | |
| DB01_F03 | GAGGTACAACTTTTGGAGTCAGGCGGC | 134 |
| P2121 | GGGTTAGTGCAGCCTGGAGGATCACTG | |
| (nucleotide) | AGGCTGAGCTGCGCCGCCTCTGGCTCA | |
| | ATTAGCAGTTTCTATTATATCGGATGGT | |
| | TCCGCCAGGCTCCGGGAAAAGAGAGA | |
| | GAGTTTGTTTCCTCTCGCATTACCTCAG | |
| | GAGGAAGCACTTACTACAGGGACTCTG | |
| | TTAAAGGACGCTTTACAATCTCCAGAG | |
| | ATAATTCCAAGAACACCTTATATCTGC | |
| | AAATGAATAGTTTGAAGCCCGAGGACA | |
| | CTGCCGTGTATTATTGCGCAGCCGGGA | |
| | CATCCCGCGATTACTACTACTGGGGAC | |
| | AAGGAACCCAGGTGACGGTCTCGAGC | |
| DB01_A11 | GAGGTACAACTTTTGGAGTCAGGCGGG | 135 |
| P2105 | GGGCTGGTGCAGCCTGGGGGATCACTG | |
| (nucleotide) | CGCCTTTCATGCGCAGCGAGTGGTTCC | |
| | ACCTCTAGTATCGGAATTATGGGCTGG | |
| | TTTCGCCAGGCTCCTGGAAAGGAAAGG | |
| | GAGCTGGTCTCCAGCATCACAGCCGGC | |
| | GGATCTACCTACTACGCCGACTCCGTT | |
| | AAGGGGCGATTCACTATCTCCCGCGAC | |
| | AATAGCAAGAACACCTTGTATCTGCAG | |
| | ATGAACTCCCTCAAACCCGAGGATACT | |
| | GCCGTGTACTATTGCAACGCACATGTG | |
| | GGCTACGGGAGGGTGCACGATGTGGAT | |
| | TACTGGGGGCAGGGGACCCAGGTGAC | |
| | GGTCTCGAGC | |
| DB01_E03 | GAGGTACAACTTTTGGAGTCAGGCGGT | 136 |
| P2120 | GGTCTCGTCCAGCCGGGGGGGAGCCTC | |
| (nucleotide) | CGCTTATCATGTGCCGCCTCCGGATTTG | |
| | TCTCACCCTCTTATATAATGGGGTGGTT | |
| | CCGGCAAGCACCAGGCAAAGAACGGG | |
| | AGTTTGTTTCCTCCGTCATAGAGTACCG | |
| | AGGGTCCACCTATTACCTGGATAGCGT | |
| | GAAGGGGCGGTTCACCATCTCCCGCGA | |
| | TAATTCCAAGAATACCCTGTACCTGCA | |
| | GATGAATAGTCTGAAACCTGAGGACAC | |
| | GGCCGTGTACTATTGCGCAGCGGGCAC | |
| | CCCCGGAGGCTACGATTACTGGGGCCA | |
| | AGGGACCCAGGTGACGGTCTCGAGC | |
| NEC_S_2 | GAGGTACAACTTTTGGAGTCAGGCGGT | 137 |
| (nucleotide) | GGACTGGTACAACCGGGTGGTTCATTG | |
| | CGTTTGAGCTGCGCTGCCTCTGGTTCTA | |
| | CCTTCTCAAGCAATGCAATGGGTTGGT | |
| | ATCGCCAAGCGCCGGGCAAAGAACGC | |
| | GAGTTGGTCTCCTCCATATCCGGTTCCG | |
| | GTGGTAGCACCCGCTACGCGGACTCGG | |
| | TAAAAGGCCGTTTTACGATCAGTCGTG | |
| | ATAATTCCAAGAATACCTTGTACCTGC | |
| | AAATGAATAGCCTTAAGCCCGAAGACA | |
| | CAGCGGTGTATTATTGTGCCTCTTATGT | |
| | CCTGTATCTCCGAGAATACTGGGGCCA | |
| | GGGTACCCAGGTGACGGTCTCGAGC | |
| NEC_S_5 | GAGGTACAACTTTTGGAGTCAGGCGGT | 138 |
| (nucleotide) | GGACTGGTACAACCGGGTGGTTCATTG | |
| | CGTTTGAGCTGCGCTGCCTCTGGTCTG | |
| | ACTTTTAGGTATAACGCAATGGGATGG | |
| | TACCGCCAAGCGCCGGGCAAAGAACG | |
| | CGAGTTTGTCTCCGCGATCAGCGGCTC | |
| | TGGGGGGGGTACCTATTACGCGGACTC | |
| | GGTAAAAGGCCGTTTTACGATCAGTCG | |
| | TGATAATTCCAAGAATACCTTGTACCT | |
| | GCAAATGAATAGCCTTAAGCCCGAAGA | |
| | CACAGCGGTGTATTATTGTGCCGCGGA | |
| | AGGCCTGTATGATTATTGGGGCCAGGG | |
| | TACCCAGGTGACGGTCTCGAGC | |
| NEC_S_11 | GAGGTACAACTTTTGGAGTCAGGCGGT | 139 |
| P3112 | GGACTGGTACAACCGGGTGGTTCATTG | |
| (nucleotide) | CGTTTGAGCTGCGCTGCCTCTGGTTTAA | |
| | CAAGCTCTGGTTACGCTATGGGCTGGT | |
| | ATCGCCAAGCGCCGGGCAAAGAACGC | |
| | GAGCTGGTGAGCAGTATTTCTTCCTCA | |
| | GGCGGACTGACCCATTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGACGCAGAT | |
| | ATTGCTTACACTGGCGCCGATTATTGG | |
| | GGCCAGGGTACCCAGGTGACGGTCTCG | |
| | AGC | |
| NEC_S_16 | GAGGTACAACTTTTGGAGTCAGGCGGT | 140 |
| (nucleotide) | GGACTGGTACAACCGGGTGGTTCATTG | |
| | CGTTTGAGCTGCGCTGCCTCTGGTTTTA | |
| | CGTTTAGCGACTATTATATGGGCTGGT | |
| | ACCGCCAAGCGCCGGGCAAAGAACGC | |
| | GAGTTCGTATCAGCTATTTCGTCCACG | |
| | GGCGGCTCCCCCTACTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCGTGGAA | |
| | GTCCCTTACGATTATTGGGGCCAGGGT | |
| | ACCCAGGTGACGGTCTCGAGC | |
| NEC_S_31 | GAGGTACAACTTTTGGAGTCAGGCGGT | 141 |
| P3113 | GGACTGGTACAACCGGGTGGTTCATTG | |
| (nucleotide) | CGTTTGAGCTGCGCTGCCTCTGGTTTTA | |
| | CGTACTCTTCTTATGCAATGGGCTGGT | |
| | ATCGCCAAGCGCCGGGCAAAGAACGC | |
| | GAGTTGGTCTCCTCCATATCCGGTTCCG | |
| | GTGGTAGCACCCGCTACGCGGACTCGG | |
| | TAAAAGGCCGTTTTACGATCAGTCGTG | |
| | ATAATTCCAAGAATACCTTGTACCTGC | |
| | AAATGAATAGCCTTAAGCCCGAAGACA | |
| | CAGCGGTGTATTATTGTGCCGTGGCGA | |
| | TTGGCGTAGGAGACTATTGGGGCCAGG | |
| | GTACCCAGGTGACGGTCTCGAGC | |
| NEC_S_55 | GAGGTACAACTTTTGGAGTCAGGCGGT | 142 |
| P3114 | GGACTGGTACAACCGGGTGGTTCATTG | |
| (nucleotide) | CGTTTGAGCTGCGCTGCCTCTGGTTTTA | |
| | CCCTGTCTTCGTATGCAATGGGCTGGT | |
| | ACCGCCAAGCGCCGGGCAAAGAACGC | |
| | GAGCTCGTTTCCAGCATCAGTGGGTCG | |
| | GGTGGCTCTACCCGGTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCGCTTAC | |
| | ATCGGCGGAGATTACTTGGGCCAGGGT | |
| | ACCCAGGTGACGGTCTCGAGC | |
| NEC_S_56 | GAGGTACAACTTTTGGAGTCAGGCGGT | 143 |
| (nucleotide) | GGACTGGTACAACCGGGTGGTTCATTG | |
| | CGTTTGAGCTGCGCTGCCTCTGGTCTG | |
| | ACACTGTCATCGTACGCAATGGGTTGG | |
| | TTCCGCCAAGCGCCGGGCAAAGAACGC | |
| | GAGCTGGTGTCCTCAATAAGCGGTAGT | |
| | GGCGGCAGCACGCGCTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCGTTGAT | |
| | ATCCGGCTGACGGATTATTGGGGCCAG | |
| | GGTACCCAGGTGACGGTCTCGAGC | |
| NEC_S_64 | GAGGTACAACTTTTGGAGTCAGGCGGT | 144 |
| (nucleotide) | GGACTGGTACAACCGGGTGGTTCATTG | |
| | CGTTTGAGCTGCGCTGCCTCTGGTTTTG | |
| | CCTACTCTTCGTATTACATGGGTTGGTA | |
| | TCGCCAAGCGCCGGGCAAAGAACGCG | |
| | AGTTTGTGAGTGCAATATCATCTAGTG | |
| | GCGGCGGTACCTACTACGCGGACTCGG | |
| | TAAAAGGCCGTTTTACGATCAGTCGTG | |
| | ATAATTCCAAGAATACCTTGTACCTGC | |
| | AAATGAATAGCCTTAAGCCCGAAGACA | |
| | CAGCGGTGTATTATTGTGCCGTTGAAC | |
| | TTACCTATAACTACTGGGGCCAGGGTA | |
| | CCCAGGTGACGGTCTCGAGC | |
| NEC_S_81 | GAGGTACAACTTTTGGAGTCAGGCGGT | 145 |
| (nucleotide) | GGACTGGTACAACCGGGTGGTTCATTG | |
| | CGTTTGAGCTGCGCTGCCTCTGGTTTCA | |
| | TATACTCTAGCTACTATATGGGATGGT | |
| | ACCGCCAAGCGCCGGGCAAAGAACGC | |
| | GAGTTTGTTAGCGCTATTACCTCATCA | |
| | GGCGGTTCTACATATTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCGTGGAA | |
| | GTCCCTGAATTTGATTATTGGGGCCAG | |
| | GGTACCCAGGTGACGGTCTCGAGC | |
| NEC_S_85 | GAGGTACAACTTTTGGAGTCAGGCGGT | 146 |
| (nucleotide) | GGACTGGTACAACCGGGTGGTTCATTG | |
| | CGTTTGAGCTGCGCTGCCTCTGGTTTCA | |
| | CATTTCGTTCTTATGCGATGGGGTGGTT | |
| | TCGCCAAGCGCCGGGCAAAGAACGCG | |
| | AGGGTGTTTCTGTGATTACAGGTACTG | |
| | GCGGTTCAACCTATTACGCGGACTCGG | |
| | TAAAAGGCCGTTTTACGATCAGTCGTG | |
| | ATAATTCCAAGAATACCTTGTACCTGC | |
| | AAATGAATAGCCTTAAGCCCGAAGACA | |
| | CAGCGGTGTATTATTGTGCCCGCGCAT | |
| | ATCTGAGTGGCTATCTGTACGAGTATT | |
| | GGGGCCAGGGTACCCAGGTGACGGTCT | |
| | CGAGC | |
| NEC_M_5 | GAGGTACAACTTTTGGAGTCAGGCGGT | 147 |
| P3106 | GGACTGGTACAACCGGGTGGTTCATTG | |
| (nucleotide) | CGTTTGAGCTGCGCTGCCTCTGGTCTG | |
| | AGCTTCTCATCTTATTCAATGGGGTGGT | |
| | ATCGCCAAGCGCCGGGCAAAGAACGC | |
| | GAGTTTGTTTCTGCAATCTCAGGCTCAT | |
| | CAGGTTCAACGAATTACGCGGACTCGG | |
| | TAAAAGGCCGTTTTACGATCAGTCGTG | |
| | ATAATTCCAAGAATACCTTGTACCTGC | |
| | AAATGAATAGCCTTAAGCCCGAAGACA | |
| | CAGCGGTGTATTATTGTGCCGCCGAAC | |
| | ACCGGGTGACCACTTCCGGAGTTTTCT | |
| | ACGACTATTGGGGCCAGGGTACCCAGG | |
| | TGACGGTCTCGAGC | |
| NEC_M_8 | GAGGTACAACTTTTGGAGTCAGGCGGT | 148 |
| P3107 | GGACTGGTACAACCGGGTGGTTCATTG | |
| (nucleotide) | CGTTTGAGCTGCGCTGCCTCTGGTTTCA | |
| | CTTTCAGCAGTTATGCGATGGGATGGT | |
| | ATCGCCAAGCGCCGGGCAAAGAACGC | |
| | GAGTTTGTTAGCAGCATAAGCGGTTCA | |
| | GGGGGGTTAACCCGGTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCGTAACC | |
| | ACCGGCTACCAAGGCGGCGTATATGAC | |
| | TACTGGGGCCAGGGTACCCAGGTGACG | |
| | GTCTCGAGC | |
| NEC_M_17 | GAGGTACAACTTTTGGAGTCAGGCGGT | 149 |
| P3108 | GGACTGGTACAACCGGGTGGTTCATTG | |
| (nucleotide) | CGTTTGAGCTGCGCTGCCTCTGGTTTCA | |
| | CTTACTCTGGGTATGCAATGGGGTGGT | |
| | ATCGCCAAGCGCCGGGCAAAGAACGC | |
| | GAGCTAGTCTCAAGCATCTCAGGTTCG | |
| | GGTACCCTTACTTCGTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGACGTAGAT | |
| | ATACCTGTGGGCGACGCTACAACCGTT | |
| | GGTGATTACTGGGGCCAGGGTACCCAG | |
| | GTGACGGTCTCGAGC | |
| NEC_M_21 | GAGGTACAACTTTTGGAGTCAGGCGGT | 150 |
| (nucleotide) | GGACTGGTACAACCGGGTGGTTCATTG | |
| | CGTTTGAGCTGCGCTGCCTCTGGTCTTA | |
| | CATTTTCTAGCAATGCTATGGGTTGGT | |
| | ACCGCCAAGCGCCGGGCAAAGAACGC | |
| | GAGCTGGTGAGTTCTATTTCGGGGAGC | |
| | GGAGGTAGTACCAATTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCGCGGAG | |
| | ATTGCTACCTACTACGATTTTGCAGATT | |
| | ACTGGGGCCAGGGTACCCAGGTGACG | |
| | GTCTCGAGC | |
| NEC_M_29 | GAGGTACAACTTTTGGAGTCAGGCGGT | 151 |
| (nucleotide) | GGACTGGTACAACCGGGTGGTTCATTG | |
| | CGTTTGAGCTGCGCTGCCTCTGGTTTTA | |
| | CCTATACGTCTTACGTCATGGGCTGGT | |
| | ATCGCCAAGCGCCGGGCAAAGAACGC | |
| | GAGCTGGTATCTGCGATCTATGGCAGT | |
| | GCAGGCAGTGGGTACTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCCGCGTT | |
| | ACTACCACAACCCACCTGTGGTCTGCC | |
| | GAGTATTGGGGCCAGGGTACCCAGGTG | |
| | ACGGTCTCGAGC | |
| NEC_M_44 | GAGGTACAACTTTTGGAGTCAGGCGGT | 152 |
| P3109 | GGACTGGTACAACCGGGTGGTTCATTG | |
| (nucleotide) | CGTTTGAGCTGCGCTGCCTCTGGTCGT | |
| | ACGCTAAGTAGTTACGCGATGGGGTGG | |
| | TACCGCCAAGCGCCGGGCAAAGAACG | |
| | CGAGCTCGTTAGTAGTATTTCCGGGAG | |
| | TGGAGGCTCTACCCGTTACGCGGACTC | |
| | GGTAAAAGGCCGTTTTACGATCAGTCG | |
| | TGATAATTCCAAGAATACCTTGTACCT | |
| | GCAAATGAATAGCCTTAAGCCCGAAGA | |
| | CACAGCGGTGTATTATTGTGCCGTCTA | |
| | CATTCTGGAGCTGGCTCCTGGCGCGGA | |
| | ATACTGGGGCCAGGGTACCCAGGTGAC | |
| | GGTCTCGAGC | |
| NEC_M_46 | GAGGTACAACTTTTGGAGTCAGGCGGT | 153 |
| P3110 | GGACTGGTACAACCGGGTGGTTCATTG | |
| (nucleotide) | CGTTTGAGCTGCGCTGCCTCTGGTTAC | |
| | ACATTTTCGGATTATGCAATGGGTTGG | |
| | TATCGCCAAGCGCCGGGCAAAGAACG | |
| | CGAGCTCGTTAGTAGTATTTCCGGGAG | |
| | TGGAGGCTCTACCCGTTACGCGGACTC | |
| | GGTAAAAGGCCGTTTTACGATCAGTCG | |
| | TGATAATTCCAAGAATACCTTGTACCT | |
| | GCAAATGAATAGCCTTAAGCCCGAAGA | |
| | CACAGCGGTGTATTATTGTGCCGCAGT | |
| | AATTCGGCAGCCTAGCACCGGTTTCTA | |
| | TGAATACTGGGGCCAGGGTACCCAGGT | |
| | GACGGTCTCGAGC | |
| NEC_M_66 | GAGGTACAACTTTTGGAGTCAGGCGGT | 154 |
| (nucleotide) | GGACTGGTACAACCGGGTGGTTCATTG | |
| | CGTTTGAGCTGCGCTGCCTCTGGTTTTG | |
| | CATATTCGAGCTACAGCATCGGCTGGT | |
| | ACCGCCAAGCGCCGGGCAAAGAACGC | |
| | GAGTTAGTCAGCGCTATCTCCGGCTCT | |
| | GGAGGCTCCTCGCGCTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCGCCATA | |
| | GCGTACGTGTACACGTCCACGGCCCGT | |
| | AATTACTGGGGCCAGGGTACCCAGGTG | |
| | ACGGTCTCGAGC | |
| NEC_M_82 | GAGGTACAACTTTTGGAGTCAGGCGGT | 155 |
| (nucleotide) | GGACTGGTACAACCGGGTGGTTCATTG | |
| | CGTTTGAGCTGCGCTGCCTCTGGTTTCA | |
| | CCTATAGCGATTATGCGATGGGATGGT | |
| | ACCGCCAAGCGCCGGGCAAAGAACGC | |
| | GAGTTGGTTTCAGCGATCTCATCGTAC | |
| | GGGGGCGTTACAAATTACGCTGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGCCGCCTAT | |
| | GGCGCTCTGTCCATTCGGGGCTATGAT | |
| | TACTGGGGCCAGGGTACCCAGGTGACG | |
| | GTCTCGAGC | |
| NEC_M_84 | GAGGTACAACTTTTGGAGTCAGGCGGT | 156 |
| (nucleotide) | GGACTGGTACAACCGGGTGGTTCATTG | |
| | CGTTTGAGCTGCGCTGCCTCTGGTTTTG | |
| | CGTTCTCCATCGACGCGATGGGTTGGT | |
| | ATCGCCAAGCGCCGGGCAAAGAACGC | |
| | GAGGGCGTTTCCTCCATATCGGGTAGC | |
| | GGTGGTCTGACACGCTACGCGGACTCG | |
| | GTAAAAGGCCGTTTTACGATCAGTCGT | |
| | GATAATTCCAAGAATACCTTGTACCTG | |
| | CAAATGAATAGCCTTAAGCCCGAAGAC | |
| | ACAGCGGTGTATTATTGTGACACAGCC | |
| | GTACTGACTGGCTATTCCGAGGCTTTT | |
| | GACTACTGGGGCCAGGGTACCCAGGTG | |
| | ACGGTCTCGAGC | |

Signaling/Co-stimulatory Domains:

| | | | |
|---|---|---|---|
| CD3-zeta | RVKFSRSADAPAYQQGQNQLYNELNLG | P20963-3 | 6 |
| isoform 3 | RREEYDVLDKRRGRDPEMGGKPRRKNP | | |
| (AA 52-163) | QEGLYNELQKDKMAEAYSEIGMKGERR | | |
| | RGKGHDGLYQGLSTATKDTYDALHMQ | | |
| | ALPPR | | |
| CD3-zeta | AGAGTGAAGTTCAGCAGATCCGCCGAT | | 101 |
| isoform 3 | GCTCCCGCCTATCAGCAGGGCCAAAAC | | |
| (nucleotide | CAGCTGTACAACGAGCTGAACCTGGGG | | |
| sequence) | AGAAGAGAAGAGTACGACGTGCTGGA | | |
| | CAAGCGGAGAGGCAGAGATCCTGAAA | | |
| | TGGGCGGCAAGCCCAGACGGAAGAAT | | |
| | CCTCAAGAGGGCCTGTATAATGAGCTG | | |
| | CAGAAAGACAAGATGGCCGAGGCCTA | | |
| | CAGCGAGATCGGAATGAAGGGCGAGC | | |
| | GCAGAAGAGGCAAGGGACACGATGGA | | |
| | CTGTACCAGGGCCTGAGCACCGCCACC | | |
| | AAGGATACCTATGATGCCCTGCACATG | | |
| | CAGGCCCTGCCTCCAAGA | | |
| 41BB | KRGRKKLLYIFKQPFMRPVQTTQEEDGC | Q07011 | 8 |
| (AA 214-255) | SCRFPEEEEGGCEL | | |
| IL2Rb | NCRNTGPWLKKVLKCNTPDPSKFFSQLS | P14784 | 9 |
| (AA 266-551) | SEHGGDVQKWLSSPFPSSSFSPGGLAPEI | | |
| | SPLEVLERDKVTQLLPLNTDAYLSLQEL | | |
| | QGQDPTHLV | | |
| CD40 | KKVAKKPTNKAPHPKQEPQEINFPDDLP | P25942 | 10 |
| (AA 216-277) | GSNTAAPVQETLHGCQPVTQEDGKESRI | | |
| | SVQERQ | | |
| OX40 | ALYLLRRDQRLPPDAHKPPGGGSFRTPIQ | P43489 | 11 |
| (AA 236-277) | EEQADAHSTLAKI | | |
| CD80 | TYCFAPRCRERRRNERLRRESVRPV | P33681 | 12 |
| (AA 264-288) | | | |
| CD86 (AA269- | KWKKKKRPRNSYKCGTNTMEREESEQT | P42081 | 13 |
| 329) | KKREKIHIPERSDEAQRVFKSSKTSSCDK | | |
| | SDTCF | | |
| CD27 | QRRKYRSNKGESPVEPAEPCHYSCPREE | P26842 | 14 |
| (AA 213-260) | EGSTIPIQEDYRKPEPACSP | | |
| ICOS | CWLTKKKYSSSVHDPNGEYMFMRAVNT | Q9Y6W8 | 15 |
| (AA 162-199) | AKKSRLTDVTL | | |
| NKG2D | MGWIRGRRSR HSWEMSEFHN | P26718 | 16 |
| (AA 1-51) | YNLDLKKSDF STRWQKQRCP | | |
| | VVKSKCRENAS | | |
| DAP10 | LCARPRRSPAQEDGKVYINMPGRG | Q9UBK5 | 17 |
| (AA 70-93) | | | |
| DAP12 | YFLGRLVPRGRGAAEAATRKQRITETES | O54885 | 18 |
| (AA 62-113) | PYQELQGQRSDVYSDLNTQRPYYK | | |
| 2B4/CD244 | WRRKRKEKQSETSPKEFLTIYEDVKDLK | Q9BZW8 | 19 |
| (AA 251-370) | TRRNHEQEQTFPGGGSTIYSMIQSQSSAP | | |
| | TSQEPAYTLYSLIQPSRKSGSRKRNHSPS | | |
| | FNSTIYEVIGKSQPKAQNPARLSRKELEN | | |
| | FDVYS | | |
| CD3-zeta | RVKFSRSADAPAYQQGQNQLYNELNLG | P20963-3 | 6 |
| isoform 3 | RREEYDVLDKRRGRDPEMGGKPRRKNP | | |
| (AA 52-163) | QEGLYNELQKDKMAEAYSEIGMKGERR | | |
| | RGKGHDGLYQGLSTATKDTYDALHMQ | | |
| | ALPPR | | |
| CD28 | RSKRSRLLHSDYMNMTPRRPGPTRKHY | P10747-1 | 20 |
| (AA 180-220) | QPYAPPRDFAAYRS | | |

Spacer/Hinge:

| | | | |
|---|---|---|---|
| CD8 | TTTPAPRPPTPAPTIASQPLSLRPEACRPA | P01732 | 21 |
| (AA 136-182) | AGGAVHTRGLDFACD | | |
| CD28 | IEVMYPPPYLDNEKSNGTIIHVKGKHLCP | P10747-1 | 22 |
| (AA 114-151) | SPLFPGPSKP | | |
| IgG4 CH3 | ESKYGPPCPPCPGQPREPQVYTLPPSQEE | | 96 |
| | MTKNQVSLTCLVKGFYPSDIAVEWESNG | | |
| | QPENNYKTTPPVLDSDGSFFLYSRLTVD | | |
| | KSRWQEGNVFSCSVMHEALHNHYTQKS | | |
| | LSLSLGK | | |

Transmembrane:

| | | | |
|---|---|---|---|
| CD28 | IEVMYPPPYLDNEKSNGTIIHVKGKHLCP | P10747-1 | 5 |
| (AA 114-220) | SPLFPGPSKPFWVLVVVGGVLACYSLLV | | |
| | TVAFIIFWVRSKRSRLLHSDYMNMTPRR | | |
| | PGPTRKHYQPYAPPRDFAAYRS | | |
| CD28 | ATCGAAGTGATGTACCCTCCACCTTAC | | 100 |
| (nucleotide | CTGGACAACGAGAAGTCCAACGGCAC | | |
| sequence) | CATCATCCACGTGAAGGGCAAGCACCT | | |
| | GTGTCCTTCTCCACTGTTCCCCGGACCT | | |
| | AGCAAGCCTTTCTGGGTGCTCGTTGTT | | |
| | GTTGGCGGCGTGCTGGCCTGTTATAGC | | |
| | CTGCTTGTGACCGTGGCCTTCATCATCT | | |
| | TTTGGGTCCGAAGCAAGCGGAGCCGGC | | |
| | TGCTGCACTCCGACTACATGAACATGA | | |
| | CCCCTAGACGGCCCGGACCAACCAGA | | |
| | AAGCACTACCAGCCTTACGCTCCTCCT | | |
| | AGAGACTTCGCCGCCTACCGGTCC | | |
| CD8 | IYIWAPLAGTCGVLLLSLVIT | P01732 | 23 |
| (AA 183-203) | | | |
| CD28 | FWVLVVVGGVLACYSLLVTVAFIIFWV | P10747-1 | 24 |
| (AA 153-179) | | | |

Linkers:

| | | |
|---|---|---|
| Whitlow Linker | GSTSGSGKPGSGEGSTKG | 3 |
| (G₄S)₃ | GGGGSGGGGSGGGGS | 25 |
| Linker 3 | GGSEGKSSGSGSESKSTGGS | 26 |
| Linker 4 | GGGSGGGS | 27 |
| Linker 5 | GGGSGGGSGGGS | 28 |
| Linker 6 | GGGSGGGSGGGSGGGS | 29 |
| Linker 7 | GGGSGGGSGGGSGGGSGGGS | 30 |
| Linker 8 | GGGGSGGGGSGGGGGGGGS | 31 |
| Linker 9 | GGGGSGGGGSGGGGSGGGGSGGGGS | 32 |
| Linker 10 | IRPRAIGGSKPRVA | 33 |
| Linker 11 | GKGGSGKGGSGKGGS | 34 |
| Linker 12 | GGKGSGGKGSGGKGS | 35 |
| Linker 13 | GGGKSGGGKSGGGKS | 36 |
| Linker 14 | GKGKSGKGKSGKGKS | 37 |
| Linker 15 | GGGKSGGKGSGKGGS | 38 |
| Linker 16 | GKPGSGKPGSGKPGS | 39 |
| Linker 17 | GKPGSGKPGSGKPGSGKPGS | 40 |
| Linker 18 | GKGKSGKGKSGKGKSGKGKS | 41 |
| Linker 19 | STAGDTHLGGEDFD | 42 |
| Linker 20 | GEGGSGEGGSGEGGS | 43 |
| Linker 21 | GGEGSGGEGSGGEGS | 44 |
| Linker 22 | GEGESGEGESGEGES | 45 |
| Linker 23 | GGGESGGEGSGEGGS | 46 |
| Linker 24 | GEGESGEGESGEGESGEGES | 47 |
| Linker 25 | GSTSGSGKPGSGEGSTKG | 48 |
| Linker 26 | PRGASKSGSASQTGSAPGS | 49 |
| Linker 27 | GTAAAGAGAAGGAAAGAAG | 50 |
| Linker 28 | GTSGSSGSGSGGSGSGGGG | 51 |
| Linker 29 | GKPGSGKPGSGKPGSGKPGS | 52 |
| Linker 30 | GSGS | 53 |
| Linker 31 | APAPAPAPAP | 54 |
| Linker 32 | APAPAPAPAPAPAPAPAPAP | 55 |
| Linker 33 | AEAAAKEAAAKEAAAAKEAAAAKEAA | 56 |
| | AAKAAA | |
| Linker 34 | GGGGS | 104 |
| Linker 35 | GGGGSGGGGS | 103 |
| Whitlow Linker | GGCTCTACAAGCGGCAGCGGCAAACCT | 99 |
| (nucleotide | GGATCTGGCGAGGGATCTACCAAGGG | |
| sequence) | C | |
| Whitlow Linker | GGCAGTACTTCTGGTAGCGGAAAACCC | 102 |
| (nucleotide | GGTAGCGGCGAGGGGTCAACTAAAGG | |
| sequence) | A | |

Signal Peptide:

| | | |
|---|---|---|
| GMCSFR Signal | MLLLVTSLLLCELPHPAFLLIP | 1 |
| Peptide | | |
| IgK Signal | MARSPAQLLGLLLLWLSGARC | 97 |
| (amino acid | | |
| sequence) | | |
| IgK Signal | ATGGCCAGATCTCCTGCTCAACTGCTGGGACTGCT | 98 |
| Peptide Variant | GCTGCTGTGGCTTAGCGGAGCCAGATGC | |
| (nucleotide | | |
| sequence) | | |
| MARS SP | ATGGCCAGATCCCCGGCACAACTGCTCGGACTCCT | 378 |
| (nucleic acid) | CCTGTTGTGGTTGAGCGGGGCCCGCTGT | |

Nectin4 Targeting CARs:

| | | |
|---|---|---|
| MARS | MARSPAQLLGLLLLWLSGARCEVQLLESGGGLVQAG | 157 |
| SP_DB01_C01_ | GSLRLSCAASGSFFSIYAMGWFRQAPGKEREFVAAYI | |
| P2112_CD8 | SSGGLTSYADSVKGRFTISRDNAKNTVYLQMNSLKPE | |
| Hinge_CD28 | DTAVYYCAADLGAQTGYVQYDYWGQGTQVTVSST | |
| tm_41BB_CD3Z | TTPAPRPPTPAPTIASQPLSLRPEACRPAAGGAVHTRG | |
| (amino acid) | LDFACDFWVLVVVGGVLACYSLLVTVAFIIFWVKRG | |
| | RKKLLYIFKQPFMRPVQTTQEEDGCSCRFPEEEEGGC | |
| | ELRVKFSRSADAPAYQQGQNQLYNELNLGRREEYDV | |
| | LDKRRGRDPEMGGKPRRKNPQEGLYNELQKDKMAE | |
| | AYSEIGMKGERRRGKGHDGLYQGLSTATKDTYDAL | |
| | HMQALPPR | |
| MARS | MARSPAQLLGLLLLWLSGARCEVQLLESGGGLVQPG | 158 |
| SP_DB01_B01_ | GSLRLSCAASGFVSSIYFMGWFRQAPGKEREFVSSSIG | |
| P2106_CD8 | KGGSTRYADSVKGRFTISRDNSKNTLYLQMNSLKPE | |
| Hinge_CD28 | DTAVYYCAGDEGLGTAHAEYDYWGQGTQVTVSSTT | |
| tm_41BB_CD3Z | TPAPRPPTPAPTIASQPLSLRPEACRPAAGGAVHTRGL | |
| (amino acid) | DFACDFWVLVVVGGVLACYSLLVTVAFIIFWVKRGR | |
| | KKLLYIFKQPFMRPVQTTQEEDGCSCRFPEEEEGGCE | |
| | LRVKFSRSADAPAYQQGQNQLYNELNLGRREEYDVL | |
| | DKRRGRDPEMGGKPRRKNPQEGLYNELQKDKMAEA | |
| | YSEIGMKGERRRGKGHDGLYQGLSTATKDTYDALH | |
| | MQALPPR | |
| MARS | MARSPAQLLGLLLLWLSGARCEVQLLESGGGLVQAG | 159 |
| SP_DB01_B10_ | GSLRLSCAASGGISEFYFMGWFRQAPGKEREFVAAEI | |
| P2110_CD8 | SPGSYTNYADSVKGRFTISRDNAKNTVYLQMNSLKP | |
| Hinge_CD28 | EDTAVYYCAADRDGDTYTAEYDYWGQGTQVTVSST | |
| tm_41BB_CD3Z | TTPAPRPPTPAPTIASQPLSLRPEACRPAAGGAVHTRG | |
| (amino acid) | LDFACDFWVLVVVGGVLACYSLLVTVAFIIFWVKRG | |
| | RKKLLYIFKQPFMRPVQTTQEEDGCSCRFPEEEEGGC | |
| | ELRVKFSRSADAPAYQQGQNQLYNELNLGRREEYDV | |
| | LDKRRGRDPEMGGKPRRKNPQEGLYNELQKDKMAE | |
| | AYSEIGMKGERRRGKGHDGLYQGLSTATKDTYDAL | |
| | HMQALPPR | |
| MARS | MARSPAQLLGLLLLWLSGARCEVQLLESGGGLVQPG | 160 |
| SP_DB01_F03 | GSLRLSCAASGSISSFYYIGWFRQAPGKEREFVSSRITS | |
| P2121_CD8 | GGSTYYRDSVKGRFTISRDNSKNTLYLQMNSLKPEDT | |
| Hinge_CD28 | AVYYCAAGTSRDYYYWGQGTQVTVSSTTTPAPRPPT | |
| tm_41BB_CD3Z | PAPTIASQPLSLRPEACRPAAGGAVHTRGLDFACDFW | |
| (amino acid) | VLVVVGGVLACYSLLVTVAFIIFWVKRGRKKLLYIFK | |
| | QPFMRPVQTTQEEDGCSCRFPEEEEGGCELRVKFSRS | |
| | ADAPAYQQGQNQLYNELNLGRREEYDVLDKRRGRD | |
| | PEMGGKPRRKNPQEGLYNELQKDKMAEAYSEIGMK | |
| | GERRRGKGHDGLYQGLSTATKDTYDALHMQALPPR | |
| MARS | MARSPAQLLGLLLLWLSGARCEVOLLESGGGLVQPG | 161 |
| SP_DB01_A11 | GSLRLSCAASGSTSSIGIMGWFRQAPGKERELVSSITA | |
| P2105_CD8 | GGSTYYADSVKGRFTISRDNSKNTLYLQMNSLKPED | |
| Hinge_CD28 | TAVYYCNAHVGYGRVHDVDYWGQGTQVTVSSTTTP | |
| tm_41BB_CD3Z | APRPPTPAPTIASQPLSLRPEACRPAAGGAVHTRGLDF | |
| (amino acid) | ACDFWVLVVVGGVLACYSLLVTVAFIIFWVKRGRKK | |
| | LLYIFKQPFMRPVQTTQEEDGCSCRFPEEEEGGCELR | |
| | VKFSRSADAPAYQQGQNQLYNELNLGRREEYDVLD | |
| | KRRGRDPEMGGKPRRKNPQEGLYNELQKDKMAEAY | |
| | SEIGMKGERRRGKGHDGLYQGLSTATKDTYDALHM | |
| | QALPPR | |
| MARS | MARSPAQLLGLLLLWLSGARCEVOLLESGGGLVQPG | 162 |
| SP DB01_E03 | GSLRLSCAASGFVSPSYIMGWFRQAPGKEREFVSSVIE | |
| P2120 CD8 | YRGSTYYLDSVKGRFTISRDNSKNTLYLQMNSLKPED | |
| Hinge_CD28 | TAVYYCAAGTPGGYDYWGQGTQVTVSSTTTPAPRPP | |
| tm 41BB_CD3Z | TPAPTIASQPLSLRPEACRPAAGGAVHTRGLDFACDF | |
| (amino acid) | WVLVVVGGVLACYSLLVTVAFIIFWVKRGRKKLLYI | |
| | FKQPFMRPVQTTQEEDGCSCRFPEEEEGGCELRVKFS | |
| | RSADAPAYQQGQNQLYNELNLGRREEYDVLDKRRG | |
| | RDPEMGGKPRRKNPQEGLYNELQKDKMAEAYSEIG | |
| | MKGERRRGKGHDGLYQGLSTATKDTYDALHMQALP | |
| | PR | |
| MARS | MARSPAQLLGLLLLWLSGARCEVQLLESGGGLVQPG | 163 |
| SP_NEC_S_11_ | GSLRLSCAASGLTSSGYAMGWYRQAPGKERELVSSIS | |
| P3112_CD8 | SSGGLTHYADSVKGRFTISRDNSKNTLYLQMNSLKPE | |
| Hinge_CD28 | DTAVYYCDADIAYTGADYWGQGTQVTVSSTTTPAPR | |
| tm_41BB_CD3Z | PPTPAPTIASQPLSLRPEACRPAAGGAVHTRGLDFAC | |
| (amino acid) | DFWVLVVVGGVLACYSLLVTVAFIIFWVKRGRKKLL | |
| | YIFKQPFMRPVQTTQEEDGCSCRFPEEEEGGCELRVK | |
| | FSRSADAPAYQQGQNQLYNELNLGRREEYDVLDKRR | |
| | GRDPEMGGKPRRKNPQEGLYNELQKDKMAEAYSEI | |
| | GMKGERRRGKGHDGLYQGLSTATKDTYDALHMQA | |
| | LPPR | |
| MARS | MARSPAQLLGLLLLWLSGARCEVQLLESGGGLVQPG | 164 |
| SP_NEC_S_31_ | GSLRLSCAASGFTYSSYAMGWYRQAPGKERELVSSIS | |
| P3113_CD8 | GSGGSTRYADSVKGRFTISRDNSKNTLYLQMNSLKPE | |
| Hinge_CD28 | DTAVYYCAVAIGVGDYWGQGTQVTVSSTTTPAPRPP | |
| tm_41BB_CD3Z | TPAPTIASQPLSLRPEACRPAAGGAVHTRGLDFACDF | |
| (amino acid) | WVLVVVGGVLACYSLLVTVAFIIFWVKRGRKKLLYI | |
| | FKQPFMRPVQTTQEEDGCSCRFPEEEEGGCELRVKFS | |
| | RSADAPAYQQGQNQLYNELNLGRREEYDVLDKRRG | |
| | RDPEMGGKPRRKNPQEGLYNELQKDKMAEAYSEIG | |
| | MKGERRRGKGHDGLYQGLSTATKDTYDALHMQALP | |
| | PR | |
| MARS | MARSPAQLLGLLLLWLSGARCEVQLLESGGGLVQPG | 165 |
| SP_NEC_S_55_ | GSLRLSCAASGFTYSSYAMGWYRQAPGKERELVSSIS | |
| P3114_CD8 | GSGGSTRYADSVKGRFTISRDNSKNTLYLQMNSLKPE | |
| Hinge_CD28 | DTAVYYCAVAIGVGDYWGQGTQVTVSSTTTPAPRPP | |
| tm_41BB_CD3Z | TPAPTIASQPLSLRPEACRPAAGGAVHTRGLDFACDF | |
| (amino acid) | WVLVVVGGVLACYSLLVTVAFIIFWVKRGRKKLLYI | |
| | FKQPFMRPVQTTQEEDGCSCRFPEEEEGGCELRVKFS | |
| | RSADAPAYQQGQNQLYNELNLGRREEYDVLDKRRG | |
| | RDPEMGGKPRRKNPQEGLYNELQKDKMAEAYSEIG | |
| | MKGERRRGKGHDGLYQGLSTATKDTYDALHMQALP | |
| | PR | |
| MARS | MARSPAQLLGLLLLWLSGARCEVOLLESGGGLVQPG | 166 |
| SP_NEC_M_5_ | GSLRLSCAASGLSFSSYSMGWYRQAPGKEREFVSAIS | |
| P3106_CD8 | GSSGSTNYADSVKGRFTISRDNSKNTLYLQMNSLKPE | |
| Hinge_CD28 | DTAVYYCAAEHRVTTSGVFYDYWGQGTQVTVSSTT | |
| tm_41BB_CD3Z | TPAPRPPTPAPTIASQPLSLRPEACRPAAGGAVHTRGL | |
| (amino acid) | DFACDFWVLVVVGGVLACYSLLVTVAFIIFWVKRGR | |
| | KKLLYIFKQPFMRPVQTTQEEDGCSCRFPEEEEGGCE | |
| | LRVKFSRSADAPAYQQGQNQLYNELNLGRREEYDVL | |
| | DKRRGRDPEMGGKPRRKNPQEGLYNELQKDKMAEA | |
| | YSEIGMKGERRRGKGHDGLYQGLSTATKDTYDALH | |
| | MQALPPR | |
| MARS | MARSPAQLLGLLLLWLSGARCEVQLLESGGGLVQPG | 167 |
| SP_NEC_M_8_ | GSLRLSCAASGFTFSSYAMGWYRQAPGKEREFVSSIS | |
| P3107_CD8 | GSGGLTRYADSVKGRFTISRDNSKNTLYLQMNSLKPE | |
| Hinge_CD28 | DTAVYYCAVTTGYQGGVYDYWGQGTQVTVSSTTTP | |
| tm_41BB_CD3Z | APRPPTPAPTIASQPLSLRPEACRPAAGGAVHTRGLDF | |
| (amino acid) | ACDFWVLVVVGGVLACYSLLVTVAFIIFWVKRGRKK | |
| | LLYIFKQPFMRPVQTTQEEDGCSCRFPEEEEGGCELR | |
| | VKFSRSADAPAYQQGQNQLYNELNLGRREEYDVLD | |
| | KRRGRDPEMGGKPRRKNPQEGLYNELQKDKMAEAY | |
| | SEIGMKGERRRGKGHDGLYQGLSTATKDTYDALHM | |
| | QALPPR | |
| MARS | MARSPAQLLGLLLLWLSGARCEVOLLESGGGLVQPG | 168 |
| SP_NEC_M_17_ | GSLRLSCAASGFTYSGYAMGWYRQAPGKERELVSSI | |
| P3108_CD8 | SGSGTLTSYADSVKGRFTISRDNSKNTLYLQMNSLKP | |
| Hinge_CD28 | EDTAVYYCDVDIPVGDATTVGDYWGQGTQVTVSST | |
| tm_41BB_CD3Z | TTPAPRPPTPAPTIASQPLSLRPEACRPAAGGAVHTRG | |
| (amino acid) | LDFACDFWVLVVVGGVLACYSLLVTVAFIIFWVKRG | |
| | RKKLLYIFKQPFMRPVQTTQEEDGCSCRFPEEEEGGC | |
| | ELRVKFSRSADAPAYQQGQNQLYNELNLGRREEYDV | |
| | LDKRRGRDPEMGGKPRRKNPQEGLYNELQKDKMAE | |
| | AYSEIGMKGERRRGKGHDGLYQGLSTATKDTYDAL | |
| | HMQALPPR | |
| MARS | MARSPAQLLGLLLLWLSGARCEVQLLESGGGLVQPG | 169 |
| SP_NEC_M_44_ | GSLRLSCAASGRTLSSYAMGWYRQAPGKERELVSSIS | |
| P3109_CD8 | GSGGSTRYADSVKGRFTISRDNSKNTLYLQMNSLKPE | |
| Hinge_CD28 | DTAVYYCAVYILELAPGAEYWGQGTQVTVSSTTTPA | |
| tm_41BB_CD3Z | PRPPTPAPTIASQPLSLRPEACRPAAGGAVHTRGLDFA | |
| (amino acid) | CDFWVLVVVGGVLACYSLLVTVAFIIFWVKRGRKKL | |
| | LYIFKQPFMRPVQTTQEEDGCSCRFPEEEEGGCELRV | |
| | KFSRSADAPAYQQGQNQLYNELNLGRREEYDVLDK | |
| | RRGRDPEMGGKPRRKNPQEGLYNELQKDKMAEAYS | |
| | EIGMKGERRRGKGHDGLYQGLSTATKDTYDALHMQ | |
| | ALPPR | |
| MARS | MARSPAQLLGLLLLWLSGARCEVQLLESGGGLVQPG | 170 |
| SP_NEC_M_46_ | GSLRLSCAASGYTFSDYAMGWYRQAPGKERELVSSI | |
| P3110_CD8 | SGSGGSTRYADSVKGRFTISRDNSKNTLYLQMNSLKP | |
| Hinge_CD28 | EDTAVYYCAAVIRQPSTGFYEYWGQGTQVTVSSTTT | |
| tm_41BB_CD3Z | PAPRPPTPAPTIASQPLSLRPEACRPAAGGAVHTRGLD | |
| (amino acid) | FACDFWVLVVVGGVLACYSLLVTVAFIIFWVKRGRK | |
| | KLLYIFKQPFMRPVQTTQEEDGCSCRFPEEEEGGCEL | |
| | RVKFSRSADAPAYQQGQNQLYNELNLGRREEYDVL | |
| | DKRRGRDPEMGGKPRRKNPQEGLYNELQKDKMAEA | |
| | YSEIGMKGERRRGKGHDGLYQGLSTATKDTYDALH | |
| | MQALPPR | |
| MARS | ATGGCCAGATCTCCTGCTCAACTGCTGGGACTGCT | 171 |
| SP_DB01_C01_ | GCTGCTGTGGCTTAGCGGAGCCAGATGCGAGGTAC | |
| P2112_CD8 | AACTTTTGGAGTCAGGCGGCGGGTTGGTCCAGGCG | |
| Hinge_CD28 | GGTGGCTCACTCCGCCTTAGTTGTGCCGCCTCAGG | |
| tm_41BB_CD3Z | GTCATTCTTTAGTATCTACGCTATGGGCTGGTTTCG | |
| (nucleotide) | ACAGGCCCCTGGTAAGGAACGTGAGTTTGTGGCCG | |
| | CCTACATTTCCTCAGGGGGGCTCACCAGCTACGCG | |
| | GATAGTGTTAAGGGTAGATTCACCATCTCCAGAGA | |
| | CAATGCAAAGAATACGGTATACCTCCAAATGAACA | |
| | GCCTGAAGCCTGAAGACACGGCTGTCTACTATTGC | |
| | GCAGCAGACTTGGGAGCCCAGACCGGATACGTTCA | |
| | GTACGACTACTGGGGGCAGGGAACCCAGGTGACG | |
| | GTCTCGAGCACAACAACTCCAGCCCCAAGACCACC | |
| | TACGCCTGCACCTACTATCGCATCTCAACCACTGTC | |
| | CCTGCGCCCTGAGGCATGCCGACCAGCAGCCGGTG | |
| | GCGCGGTGCATACCCGCGGACTGGACTTTGCCTGC | |
| | GATTTTTGGGTGCTGGTGGTGGTTGGTGGAGTCCTG | |
| | GCTTGCTATAGCTTGCTAGTAACAGTGGCCTTTATT | |
| | ATTTTCTGGGTGAAACGGGGCAGAAAGAAACTCCT | |
| | GTATATATTCAAACAACCATTTATGCGACCAGTAC | |
| | AAACTACTCAAGAGGAAGATGGCTGTAGCTGCCGA | |
| | TTTCCAGAAGAAGAAGAAGGAGGATGTGAACTGA | |
| | GAGTGAAGTTCAGCAGGAGCGCAGACGCCCCCGC | |
| | GTACCAGCAGGGCCAGAACCAGCTCTATAACGAGC | |
| | TCAATCTAGGACGAAGAGAGGAGTACGATGTTTTG | |
| | GACAAGCGACGTGGCCGGGACCCTGAGATGGGGG | |
| | GAAAGCCGAGAAGGAAGAACCCTCAGGAAGGCCT | |
| | GTACAATGAACTGCAGAAAGATAAGATGGCGGAG | |
| | GCCTACAGTGAGATTGGGATGAAAGGCGAGCGCC | |
| | GGAGGGGCAAGGGGCACGATGGCCTTTACCAGGG | |
| | ACTCAGTACAGCCACCAAGGACACCTACGACGCCC | |
| | TTCACATGCAGGCCCTGCCCCCTCGC | |
| MARS | ATGGCCAGATCTCCTGCTCAACTGCTGGGACTGCT | 172 |
| SP_DB01_B01_ | GCTGCTGTGGCTTAGCGGAGCCAGATGCGAGGTAC | |
| P2106_CD8 | AACTTTTGGAGTCAGGCGGCGGCCTGGTGCAGCCC | |
| Hinge_CD28 | GGAGGGAGTCTCCGACTGTCTTGCGCTGCATCTGG | |
| tm_41BB_CD3Z | ATTCGTGAGCTCTATATACTTTATGGGATGGTTCAG | |
| (nucleotide) | GCAGGCTCCTGGGAAGGAGCGCGAGTTTGTGTCTA | |
| | GTAGTATTGGCAAGGGTGGCTCAACACGCTATGCG | |
| | GATTCTGTGAAAGGGAGGTTCACAATAAGCAGGGA | |
| | CAACTCAAAGAATACACTGTACCTCCAGATGAACT | |
| | CCTTAAAACCAGAGGATACTGCAGTCTATTACTGT | |
| | GCTGGAGATGAGGGATTGGGAACTGCACATGCTGA | |
| | ATACGACTACTGGGGCCAGGGGACCCAGGTGACG | |
| | GTCTCGAGCACAACAACTCCAGCCCCAAGACCACC | |
| | TACGCCTGCACCTACTATCGCATCTCAACCACTGTC | |
| | CCTGCGCCCTGAGGCATGCCGACCAGCAGCCGGTG | |
| | GCGCGGTGCATACCCGCGGACTGGACTTTGCCTGC | |
| | GATTTTTGGGTGCTGGTGGTGGTTGGTGGAGTCCTG | |
| | GCTTGCTATAGCTTGCTAGTAACAGTGGCCTTTATT | |
| | ATTTTCTGGGTGAAACGGGGCAGAAAGAAACTCCT | |
| | GTATATATTCAAACAACCATTTATGCGACCAGTAC | |
| | AAACTACTCAAGAGGAAGATGGCTGTAGCTGCCGA | |
| | TTTCCAGAAGAAGAAGAAGGAGGATGTGAACTGA | |
| | GAGTGAAGTTCAGCAGGAGCGCAGACGCCCCCGC | |
| | GTACCAGCAGGGCCAGAACCAGCTCTATAACGAGC | |
| | TCAATCTAGGACGAAGAGAGGAGTACGATGTTTTG | |
| | GACAAGCGACGTGGCCGGGACCCTGAGATGGGGG | |
| | GAAAGCCGAGAAGGAAGAACCCTCAGGAAGGCCT | |
| | GTACAATGAACTGCAGAAAGATAAGATGGCGGAG | |
| | GCCTACAGTGAGATTGGGATGAAAGGCGAGCGCC | |
| | GGAGGGGCAAGGGGCACGATGGCCTTTACCAGGG | |
| | ACTCAGTACAGCCACCAAGGACACCTACGACGCCC | |
| | TTCACATGCAGGCCCTGCCCCCTCGC | |
| MARS | ATGGCCAGATCTCCTGCTCAACTGCTGGGACTGCT | 173 |
| SP_DB01_B10_ | GCTGCTGTGGCTTAGCGGAGCCAGATGCGAGGTAC | |
| P2110_CD8 | AACTTTTGGAGTCAGGCGGCGGGTTGGTCCAGGCG | |
| Hinge_CD28 | GGTGGCTCACTCCGCCTTAGTTGTGCCGCCTCAGG | |
| tm_41BB_CD3Z | GTCATTCTTTAGTATCTACGCTATGGGCTGGTTTCG | |
| (nucleotide) | ACAGGCCCCTGGTAAGGAACGTGAGTTTGTGGCCG | |
| | CCTACATTTCCTCAGGGGGGCTCACCAGCTACGCG | |
| | GATAGTGTTAAGGGTAGATTCACCATCTCCAGAGA | |
| | CAATGCAAAGAATACGGTATACCTCCAAATGAACA | |
| | GCCTGAAGCCTGAAGACACGGCTGTCTACTATTGC | |
| | GCAGCAGACTTGGGAGCCCAGACCGGATACGTTCA | |
| | GTACGACTACTGGGGGCAGGGAACCCAGGTGACG | |
| | GTCTCGAGCACAACAACTCCAGCCCCAAGACCACC | |
| | TACGCCTGCACCTACTATCGCATCTCAACCACTGTC | |
| | CCTGCGCCCTGAGGCATGCCGACCAGCAGCCGGTG | |
| | GCGCGGTGCATACCCGCGGACTGGACTTTGCCTGC | |
| | GATTTTTGGGTGCTGGTGGTGGTTGGTGGAGTCCTG | |
| | GCTTGCTATAGCTTGCTAGTAACAGTGGCCTTTATT | |
| | ATTTTCTGGGTGAAACGGGGCAGAAAGAAACTCCT | |
| | GTATATATTCAAACAACCATTTATGCGACCAGTAC | |
| | AAACTACTCAAGAGGAAGATGGCTGTAGCTGCCGA | |
| | TTTCCAGAAGAAGAAGAAGGAGGATGTGAACTGA | |
| | GAGTGAAGTTCAGCAGGAGCGCAGACGCCCCCGC | |
| | GTACCAGCAGGGCCAGAACCAGCTCTATAACGAGC | |
| | TCAATCTAGGACGAAGAGAGGAGTACGATGTTTTG | |
| | GACAAGCGACGTGGCCGGGACCCTGAGATGGGGG | |
| | GAAAGCCGAGAAGGAAGAACCCTCAGGAAGGCCT | |
| | GTACAATGAACTGCAGAAAGATAAGATGGCGGAG | |
| | GCCTACAGTGAGATTGGGATGAAAGGCGAGCGCC | |
| | GGAGGGGCAAGGGGCACGATGGCCTTTACCAGGG | |
| | ACTCAGTACAGCCACCAAGGACACCTACGACGCCC | |
| | TTCACATGCAGGCCCTGCCCCCTCGC | |
| MARS | ATGGCCAGATCTCCTGCTCAACTGCTGGGACTGCT | 174 |
| SP_DB01_F03_ | GCTGCTGTGGCTTAGCGGAGCCAGATGCGAGGTAC | |
| P2121_CD8 | AACTTTTGGAGTCAGGCGGCGGGTTAGTGCAGCCT | |
| Hinge_CD28 | GGAGGATCACTGAGGCTGAGCTGCGCCGCCTCTGG | |
| tm_41BB_CD3Z | CTCAATTAGCAGTTTCTATTATATCGGATGGTTCCG | |
| (nucleotide) | CCAGGCTCCGGGAAAAGAGAGAGAGTTTGTTTCCT | |
| | CTCGCATTACCTCAGGAGGAAGCACTTACTACAGG | |
| | GACTCTGTTAAAGGACGCTTTACAATCTCCAGAGA | |
| | TAATTCCAAGAACACCTTATATCTGCAAATGAATA | |
| | GTTTGAAGCCCGAGGACACTGCCGTGTATTATTGC | |
| | GCAGCCGGGACATCCCGCGATTACTACTACTGGGG | |
| | ACAAGGAACCCAGGTGACGGTCTCGAGCACAACA | |
| | ACTCCAGCCCCAAGACCACCTACGCCTGCACCTAC | |
| | TATCGCATCTCAACCACTGTCCCTGCGCCCTGAGGC | |
| | ATGCCGACCAGCAGCCGGTGGCGCGGTGCATACCC | |
| | GCGGACTGGACTTTGCCTGCGATTTTTGGGTGCTGG | |
| | TGGTGGTTGGTGGAGTCCTGGCTTGCTATAGCTTGC | |
| | TAGTAACAGTGGCCTTTATTATTTTCTGGGTGAAAC | |
| | GGGGCAGAAAGAAACTCCTGTATATATTCAAACAA | |
| | CCATTTATGCGACCAGTACAAACTACTCAAGAGGA | |
| | AGATGGCTGTAGCTGCCGATTTCCAGAAGAAGAAG | |
| | AAGGAGGATGTGAACTGAGAGTGAAGTTCAGCAG | |
| | GAGCGCAGACGCCCCCGCGTACCAGCAGGGCCAG | |
| | AACCAGCTCTATAACGAGCTCAATCTAGGACGAAG | |
| | AGAGGAGTACGATGTTTTGGACAAGCGACGTGGCC | |
| | GGGACCCTGAGATGGGGGGAAAGCCGAGAAGGAA | |
| | GAACCCTCAGGAAGGCCTGTACAATGAACTGCAGA | |
| | AAGATAAGATGGCGGAGGCCTACAGTGAGATTGG | |
| | GATGAAAGGCGAGCGCCGGAGGGGCAAGGGGCAC | |
| | GATGGCCTTTACCAGGGACTCAGTACAGCCACCAA | |
| | GGACACCTACGACGCCCTTCACATGCAGGCCCTGC | |
| | CCCCTCGC | |
| MARS | ATGGCCAGATCTCCTGCTCAACTGCTGGGACTGCT | 175 |
| SP_DB01_A11_ | GCTGCTGTGGCTTAGCGGAGCCAGATGCGAGGTAC | |
| P2105_CD8 | AACTTTTGGAGTCAGGCGGGGGGCTGGTGCAGCCT | |
| Hinge_CD28 | GGGGGATCACTGCGCCTTTCATGCGCAGCGAGTGG | |
| tm_41BB_CD3Z | TTCCACCTCTAGTATCGGAATTATGGGCTGGTTTCG | |
| (nucleotide) | CCAGGCTCCTGGAAAGGAAAGGGAGCTGGTCTCCA | |
| | GCATCACAGCCGGCGGATCTACCTACTACGCCGAC | |
| | TCCGTTAAGGGGCGATTCACTATCTCCCGCGACAA | |
| | TAGCAAGAACACCTTGTATCTGCAGATGAACTCCC | |
| | TCAAACCCGAGGATACTGCCGTGTACTATTGCAAC | |
| | GCACATGTGGGCTACGGGAGGGTGCACGATGTGGA | |
| | TTACTGGGGGCAGGGGACCCAGGTGACGGTCTCGA | |
| | GCACAACAACTCCAGCCCCAAGACCACCTACGCCT | |
| | GCACCTACTATCGCATCTCAACCACTGTCCCTGCGC | |
| | CCTGAGGCATGCCGACCAGCAGCCGGTGGCGCGGT | |
| | GCATACCCGCGGACTGGACTTTGCCTGCGATTTTTG | |
| | GGTGCTGGTGGTGGTTGGTGGAGTCCTGGCTTGCT | |
| | ATAGCTTGCTAGTAACAGTGGCCTTTATTATTTTCT | |
| | GGGTGAAACGGGGCAGAAAGAAACTCCTGTATAT | |
| | ATTCAAACAACCATTTATGCGACCAGTACAAACTA | |
| | CTCAAGAGGAAGATGGCTGTAGCTGCCGATTTCCA | |
| | GAAGAAGAAGAAGGAGGATGTGAACTGAGAGTGA | |
| | AGTTCAGCAGGAGCGCAGACGCCCCCGCGTACCAG | |
| | CAGGGCCAGAACCAGCTCTATAACGAGCTCAATCT | |
| | AGGACGAAGAGAGGAGTACGATGTTTTGGACAAG | |
| | CGACGTGGCCGGGACCCTGAGATGGGGGGAAAGC | |
| | CGAGAAGGAAGAACCCTCAGGAAGGCCTGTACAA | |
| | TGAACTGCAGAAAGATAAGATGGCGGAGGCCTAC | |
| | AGTGAGATTGGGATGAAAGGCGAGCGCCGGAGGG | |
| | GCAAGGGGCACGATGGCCTTTACCAGGGACTCAGT | |
| | ACAGCCACCAAGGACACCTACGACGCCCTTCACAT | |
| | GCAGGCCCTGCCCCCTCGC | |
| MARS | ATGGCCAGATCTCCTGCTCAACTGCTGGGACTGCT | 176 |
| SP_DB01_E03_ | GCTGCTGTGGCTTAGCGGAGCCAGATGCGAGGTAC | |
| P2120_CD8 | AACTTTTGGAGTCAGGCGGTGGTCTCGTCCAGCCG | |
| Hinge_CD28 | GGGGGGAGCCTCCGCTTATCATGTGCCGCCTCCGG | |
| tm_41BB_CD3Z | ATTTGTCTCACCCTCTTATATAATGGGGTGGTTCCG | |
| (nucleotide) | GCAAGCACCAGGCAAAGAACGGGAGTTTGTTTCCT | |
| | CCGTCATAGAGTACCGAGGGTCCACCTATTACCTG | |
| | GATAGCGTGAAGGGGCGGTTCACCATCTCCCGCGA | |
| | TAATTCCAAGAATACCCTGTACCTGCAGATGAATA | |
| | GTCTGAAACCTGAGGACACGGCCGTGTACTATTGC | |
| | GCAGCGGGCACCCCCGGAGGCTACGATTACTGGGG | |
| | CCAAGGGACCCAGGTGACGGTCTCGAGCACAACA | |
| | ACTCCAGCCCCAAGACCACCTACGCCTGCACCTAC | |
| | TATCGCATCTCAACCACTGTCCCTGCGCCCTGAGGC | |
| | ATGCCGACCAGCAGCCGGTGGCGCGGTGCATACCC | |
| | GCGGACTGGACTTTGCCTGCGATTTTTGGGTGCTGG | |
| | TGGTGGTTGGTGGAGTCCTGGCTTGCTATAGCTTGC | |
| | TAGTAACAGTGGCCTTTATTATTTTCTGGGTGAAAC | |
| | GGGGCAGAAAGAAACTCCTGTATATATTCAAACAA | |
| | CCATTTATGCGACCAGTACAAACTACTCAAGAGGA | |
| | AGATGGCTGTAGCTGCCGATTTCCAGAAGAAGAAG | |
| | AAGGAGGATGTGAACTGAGAGTGAAGTTCAGCAG | |
| | GAGCGCAGACGCCCCCGCGTACCAGCAGGGCCAG | |
| | AACCAGCTCTATAACGAGCTCAATCTAGGACGAAG | |
| | AGAGGAGTACGATGTTTTGGACAAGCGACGTGGCC | |
| | GGGACCCTGAGATGGGGGGAAAGCCGAGAAGGAA | |
| | GAACCCTCAGGAAGGCCTGTACAATGAACTGCAGA | |
| | AAGATAAGATGGCGGAGGCCTACAGTGAGATTGG | |
| | GATGAAAGGCGAGCGCCGGAGGGGCAAGGGGCAC | |
| | GATGGCCTTTACCAGGGACTCAGTACAGCCACCAA | |
| | GGACACCTACGACGCCCTTCACATGCAGGCCCTGC | |
| | CCCCTCGC | |
| MARS | ATGGCCAGATCTCCTGCTCAACTGCTGGGACTGCT | 177 |
| SP_NEC_S_11_ | GCTGCTGTGGCTTAGCGGAGCCAGATGCGAGGTAC | |
| P3112_CD8 | AACTTTTGGAGTCAGGCGGTGGACTGGTACAACCG | |
| Hinge_CD28 | GGTGGTTCATTGCGTTTGAGCTGCGCTGCCTCTGGT | |
| tm_41BB_CD3Z | TTAACAAGCTCTGGTTACGCTATGGGCTGGTATCG | |
| (nucleotide) | CCAAGCGCCGGGCAAAGAACGCGAGCTGGTGAGC | |
| | AGTATTTCTTCCTCAGGCGGACTGACCCATTACGCG | |
| | GACTCGGTAAAAGGCCGTTTTACGATCAGTCGTGA | |
| | TAATTCCAAGAATACCTTGTACCTGCAAATGAATA | |
| | GCCTTAAGCCCGAAGACACAGCGGTGTATTATTGT | |
| | GACGCAGATATTGCTTACACTGGCGCCGATTATTG | |
| | GGGCCAGGGTACCCAGGTGACGGTCTCGAGCACAA | |
| | CAACTCCAGCCCCAAGACCACCTACGCCTGCACCT | |
| | ACTATCGCATCTCAACCACTGTCCCTGCGCCCTGAG | |
| | GCATGCCGACCAGCAGCCGGTGGCGCGGTGCATAC | |
| | CCGCGGACTGGACTTTGCCTGCGATTTTTGGGTGCT | |
| | GGTGGTGGTTGGTGGAGTCCTGGCTTGCTATAGCTT | |
| | GCTAGTAACAGTGGCCTTTATTATTTTCTGGGTGAA | |
| | ACGGGGCAGAAAGAAACTCCTGTATATATTCAAAC | |
| | AACCATTTATGCGACCAGTACAAACTACTCAAGAG | |
| | GAAGATGGCTGTAGCTGCCGATTTCCAGAAGAAGA | |
| | AGAAGGAGGATGTGAACTGAGAGTGAAGTTCAGC | |
| | AGGAGCGCAGACGCCCCCGCGTACCAGCAGGGCC | |
| | AGAACCAGCTCTATAACGAGCTCAATCTAGGACGA | |
| | AGAGAGGAGTACGATGTTTTGGACAAGCGACGTGG | |
| | CCGGGACCCTGAGATGGGGGGAAAGCCGAGAAGG | |
| | AAGAACCCTCAGGAAGGCCTGTACAATGAACTGCA | |
| | GAAAGATAAGATGGCGGAGGCCTACAGTGAGATT | |
| | GGGATGAAAGGCGAGCGCCGGAGGGGCAAGGGGC | |
| | ACGATGGCCTTTACCAGGGACTCAGTACAGCCACC | |
| | AAGGACACCTACGACGCCCTTCACATGCAGGCCCT | |
| | GCCCCCTCGC | |
| MARS | ATGGCCAGATCTCCTGCTCAACTGCTGGGACTGCT | |
| SP_NEC_S_31_ | GCTGCTGTGGCTTAGCGGAGCCAGATGCGAGGTAC | |
| P3113_CD8 | AACTTTTGGAGTCAGGCGGTGGACTGGTACAACCG | |
| Hinge_CD28 | GGTGGTTCATTGCGTTTGAGCTGCGCTGCCTCTGGT | |
| tm_41BB_CD3Z | TTTACGTACTCTTCTTATGCAATGGGCTGGTATCGC | |
| (nucleotide) | CAAGCGCCGGGCAAAGAACGCGAGTTGGTCTCCTC | |
| | CATATCCGGTTCCGGTGGTAGCACCCGCTACGCGG | |
| | ACTCGGTAAAAGGCCGTTTTACGATCAGTCGTGAT | |
| | AATTCCAAGAATACCTTGTACCTGCAAATGAATAG | |
| | CCTTAAGCCCGAAGACACAGCGGTGTATTATTGTG | |
| | CCGTGGCGATTGGCGTAGGAGACTATTGGGGCCAG | |
| | GGTACCCAGGTGACGGTCTCGAGCACAACAACTCC | |
| | AGCCCCAAGACCACCTACGCCTGCACCTACTATCG | |
| | CATCTCAACCACTGTCCCTGCGCCCTGAGGCATGC | |
| | CGACCAGCAGCCGGTGGCGCGGTGCATACCCGCGG | |
| | ACTGGACTTTGCCTGCGATTTTTGGGTGCTGGTGGT | |
| | GGTTGGTGGAGTCCTGGCTTGCTATAGCTTGCTAGT | 178 |
| | AACAGTGGCCTTTATTATTTTCTGGGTGAAACGGG | |
| | GCAGAAAGAAACTCCTGTATATATTCAAACAACCA | |
| | TTTATGCGACCAGTACAAACTACTCAAGAGGAAGA | |
| | TGGCTGTAGCTGCCGATTTCCAGAAGAAGAAGAAG | |
| | GAGGATGTGAACTGAGAGTGAAGTTCAGCAGGAG | |
| | CGCAGACGCCCCCGCGTACCAGCAGGGCCAGAACC | |
| | AGCTCTATAACGAGCTCAATCTAGGACGAAGAGAG | |
| | GAGTACGATGTTTTGGACAAGCGACGTGGCCGGGA | |
| | CCCTGAGATGGGGGGAAAGCCGAGAAGGAAGAAC | |
| | CCTCAGGAAGGCCTGTACAATGAACTGCAGAAAGA | |
| | TAAGATGGCGGAGGCCTACAGTGAGATTGGGATGA | |
| | AAGGCGAGCGCCGGAGGGGCAAGGGGCACGATGG | |
| | CCTTTACCAGGGACTCAGTACAGCCACCAAGGACA | |
| | CCTACGACGCCCTTCACATGCAGGCCCTGCCCCCTC | |
| | GC | |
| MARS | ATGGCCAGATCTCCTGCTCAACTGCTGGGACTGCT | 179 |
| SP_NEC_S_55_ | GCTGCTGTGGCTTAGCGGAGCCAGATGCGAGGTAC | |
| P3114_CD8 | AACTTTTGGAGTCAGGCGGTGGACTGGTACAACCG | |
| Hinge_CD28 | GGTGGTTCATTGCGTTTGAGCTGCGCTGCCTCTGGT | |
| tm_41BB_CD3Z | TTTACCCTGTCTTCGTATGCAATGGGCTGGTACCGC | |
| (nucleotide) | CAAGCGCCGGGCAAAGAACGCGAGCTCGTTTCCAG | |
| | CATCAGTGGGTCGGGTGGCTCTACCCGGTACGCGG | |
| | ACTCGGTAAAAGGCCGTTTTACGATCAGTCGTGAT | |
| | AATTCCAAGAATACCTTGTACCTGCAAATGAATAG | |
| | CCTTAAGCCCGAAGACACAGCGGTGTATTATTGTG | |
| | CCGCTTACATCGGCGGAGATTACTTGGGCCAGGGT | |
| | ACCCAGGTGACGGTCTCGAGCACAACAACTCCAGC | |
| | CCCAAGACCACCTACGCCTGCACCTACTATCGCAT | |
| | CTCAACCACTGTCCCTGCGCCCTGAGGCATGCCGA | |
| | CCAGCAGCCGGTGGCGCGGTGCATACCCGCGGACT | |
| | GGACTTTGCCTGCGATTTTTGGGTGCTGGTGGTGGT | |
| | TGGTGGAGTCCTGGCTTGCTATAGCTTGCTAGTAAC | |
| | AGTGGCCTTTATTATTTTCTGGGTGAAACGGGGCA | |
| | GAAAGAAACTCCTGTATATATTCAAACAACCATTT | |
| | ATGCGACCAGTACAAACTACTCAAGAGGAAGATG | |
| | GCTGTAGCTGCCGATTTCCAGAAGAAGAAGAAGGA | |
| | GGATGTGAACTGAGAGTGAAGTTCAGCAGGAGCG | |
| | CAGACGCCCCCGCGTACCAGCAGGGCCAGAACCA | |
| | GCTCTATAACGAGCTCAATCTAGGACGAAGAGAGG | |
| | AGTACGATGTTTTGGACAAGCGACGTGGCCGGGAC | |
| | CCTGAGATGGGGGGAAAGCCGAGAAGGAAGAACC | |
| | CTCAGGAAGGCCTGTACAATGAACTGCAGAAAGAT | |
| | AAGATGGCGGAGGCCTACAGTGAGATTGGGATGA | |
| | AAGGCGAGCGCCGGAGGGGCAAGGGGCACGATGG | |
| | CCTTTACCAGGGACTCAGTACAGCCACCAAGGACA | |
| | CCTACGACGCCCTTCACATGCAGGCCCTGCCCCCTC | |
| | GC | |
| MARS | ATGGCCAGATCTCCTGCTCAACTGCTGGGACTGCT | 180 |
| SP_NEC_M_5_ | GCTGCTGTGGCTTAGCGGAGCCAGATGCGAGGTAC | |
| P3106_CD8 | AACTTTTGGAGTCAGGCGGTGGACTGGTACAACCG | |
| Hinge_CD28 | GGTGGTTCATTGCGTTTGAGCTGCGCTGCCTCTGGT | |
| tm_41BB_CD3Z | CTGAGCTTCTCATCTTATTCAATGGGGTGGTATCGC | |
| (nucleotide) | CAAGCGCCGGGCAAAGAACGCGAGTTTGTTTCTGC | |
| | AATCTCAGGCTCATCAGGTTCAACGAATTACGCGG | |
| | ACTCGGTAAAAGGCCGTTTTACGATCAGTCGTGAT | |
| | AATTCCAAGAATACCTTGTACCTGCAAATGAATAG | |
| | CCTTAAGCCCGAAGACACAGCGGTGTATTATTGTG | |
| | CCGCCGAACACCGGGTGACCACTTCCGGAGTTTTC | |
| | TACGACTATTGGGGCCAGGGTACCCAGGTGACGGT | |
| | CTCGAGCACAACAACTCCAGCCCCAAGACCACCTA | |
| | CGCCTGCACCTACTATCGCATCTCAACCACTGTCCC | |
| | TGCGCCCTGAGGCATGCCGACCAGCAGCCGGTGGC | |
| | GCGGTGCATACCCGCGGACTGGACTTTGCCTGCGA | |
| | TTTTTGGGTGCTGGTGGTGGTTGGTGGAGTCCTGGC | |
| | TTGCTATAGCTTGCTAGTAACAGTGGCCTTTATTAT | |
| | TTTCTGGGTGAAACGGGGCAGAAAGAAACTCCTGT | |
| | ATATATTCAAACAACCATTTATGCGACCAGTACAA | |
| | ACTACTCAAGAGGAAGATGGCTGTAGCTGCCGATT | |
| | TCCAGAAGAAGAAGAAGGAGGATGTGAACTGAGA | |
| | GTGAAGTTCAGCAGGAGCGCAGACGCCCCCGCGTA | |
| | CCAGCAGGGCCAGAACCAGCTCTATAACGAGCTCA | |
| | ATCTAGGACGAAGAGAGGAGTACGATGTTTTGGAC | |
| | AAGCGACGTGGCCGGGACCCTGAGATGGGGGGAA | |
| | AGCCGAGAAGGAAGAACCCTCAGGAAGGCCTGTA | |
| | CAATGAACTGCAGAAAGATAAGATGGCGGAGGCC | |
| | TACAGTGAGATTGGGATGAAAGGCGAGCGCCGGA | |
| | GGGGCAAGGGGCACGATGGCCTTTACCAGGGACTC | |
| | AGTACAGCCACCAAGGACACCTACGACGCCCTTCA | |
| | CATGCAGGCCCTGCCCCCTCGC | |
| MARS | ATGGCCAGATCTCCTGCTCAACTGCTGGGACTGCT | 181 |
| SP_NEC_M_8_ | GCTGCTGTGGCTTAGCGGAGCCAGATGCGAGGTAC | |
| P3107_CD8 | AACTTTTGGAGTCAGGCGGTGGACTGGTACAACCG | |
| Hinge_CD28 | GGTGGTTCATTGCGTTTGAGCTGCGCTGCCTCTGGT | |
| tm_41BB_CD3Z | TTCACTTTCAGCAGTTATGCGATGGGATGGTATCGC | |
| (nucleotide) | CAAGCGCCGGGCAAAGAACGCGAGTTTGTTAGCAG | |
| | CATAAGCGGTTCAGGGGGGTTAACCCGGTACGCGG | |
| | ACTCGGTAAAAGGCCGTTTTACGATCAGTCGTGAT | |
| | AATTCCAAGAATACCTTGTACCTGCAAATGAATAG | |
| | CCTTAAGCCCGAAGACACAGCGGTGTATTATTGTG | |
| | CCGTAACCACCGGCTACCAAGGCGGCGTATATGAC | |
| | TACTGGGGCCAGGGTACCCAGGTGACGGTCTCGAG | |
| | CACAACAACTCCAGCCCCAAGACCACCTACGCCTG | |
| | CACCTACTATCGCATCTCAACCACTGTCCCTGCGCC | |
| | CTGAGGCATGCCGACCAGCAGCCGGTGGCGCGGTG | |
| | CATACCCGCGGACTGGACTTTGCCTGCGATTTTTGG | |
| | GTGCTGGTGGTGGTTGGTGGAGTCCTGGCTTGCTAT | |
| | AGCTTGCTAGTAACAGTGGCCTTTATTATTTTCTGG | |
| | GTGAAACGGGGCAGAAAGAAACTCCTGTATATATT | |
| | CAAACAACCATTTATGCGACCAGTACAAACTACTC | |
| | AAGAGGAAGATGGCTGTAGCTGCCGATTTCCAGAA | |
| | GAAGAAGAAGGAGGATGTGAACTGAGAGTGAAGT | |
| | TCAGCAGGAGCGCAGACGCCCCCGCGTACCAGCAG | |
| | GGCCAGAACCAGCTCTATAACGAGCTCAATCTAGG | |
| | ACGAAGAGAGGAGTACGATGTTTTGGACAAGCGA | |
| | CGTGGCCGGGACCCTGAGATGGGGGGAAAGCCGA | |
| | GAAGGAAGAACCCTCAGGAAGGCCTGTACAATGA | |
| | ACTGCAGAAAGATAAGATGGCGGAGGCCTACAGT | |
| | GAGATTGGGATGAAAGGCGAGCGCCGGAGGGGCA | |
| | AGGGGCACGATGGCCTTTACCAGGGACTCAGTACA | |
| | GCCACCAAGGACACCTACGACGCCCTTCACATGCA | |
| | GGCCCTGCCCCCTCGC | |
| MARS | ATGGCCAGATCTCCTGCTCAACTGCTGGGACTGCT | 182 |
| SP_NEC_M_17_ | GCTGCTGTGGCTTAGCGGAGCCAGATGCGAGGTAC | |
| P3108_CD8 | AACTTTTGGAGTCAGGCGGTGGACTGGTACAACCG | |
| Hinge_CD28 | GGTGGTTCATTGCGTTTGAGCTGCGCTGCCTCTGGT | |
| tm_41BB_CD3Z | TTCACTTACTCTGGGTATGCAATGGGGTGGTATCGC | |
| (nucleotide) | CAAGCGCCGGGCAAAGAACGCGAGCTAGTCTCAA | |
| | GCATCTCAGGTTCGGGTACCCTTACTTCGTACGCGG | |
| | ACTCGGTAAAAGGCCGTTTTACGATCAGTCGTGAT | |
| | AATTCCAAGAATACCTTGTACCTGCAAATGAATAG | |
| | CCTTAAGCCCGAAGACACAGCGGTGTATTATTGTG | |
| | ACGTAGATATACCTGTGGGCGACGCTACAACCGTT | |
| | GGTGATTACTGGGGCCAGGGTACCCAGGTGACGGT | |
| | CTCGAGCACAACAACTCCAGCCCCAAGACCACCTA | |
| | CGCCTGCACCTACTATCGCATCTCAACCACTGTCCC | |
| | TGCGCCCTGAGGCATGCCGACCAGCAGCCGGTGGC | |
| | GCGGTGCATACCCGCGGACTGGACTTTGCCTGCGA | |
| | TTTTTGGGTGCTGGTGGTGGTTGGTGGAGTCCTGGC | |
| | TTGCTATAGCTTGCTAGTAACAGTGGCCTTTATTAT | |
| | TTTCTGGGTGAAACGGGGCAGAAAGAAACTCCTGT | |
| | ATATATTCAAACAACCATTTATGCGACCAGTACAA | |
| | ACTACTCAAGAGGAAGATGGCTGTAGCTGCCGATT | |
| | TCCAGAAGAAGAAGAAGGAGGATGTGAACTGAGA | |
| | GTGAAGTTCAGCAGGAGCGCAGACGCCCCCGCGTA | |
| | CCAGCAGGGCCAGAACCAGCTCTATAACGAGCTCA | |
| | ATCTAGGACGAAGAGAGGAGTACGATGTTTTGGAC | |
| | AAGCGACGTGGCCGGGACCCTGAGATGGGGGGAA | |
| | AGCCGAGAAGGAAGAACCCTCAGGAAGGCCTGTA | |
| | CAATGAACTGCAGAAAGATAAGATGGCGGAGGCC | |
| | TACAGTGAGATTGGGATGAAAGGCGAGCGCCGGA | |
| | GGGGCAAGGGGCACGATGGCCTTTACCAGGGACTC | |
| | AGTACAGCCACCAAGGACACCTACGACGCCCTTCA | |
| | CATGCAGGCCCTGCCCCCTCGC | |
| MARS | ATGGCCAGATCTCCTGCTCAACTGCTGGGACTGCT | 183 |
| SP_NEC_M_44_ | GCTGCTGTGGCTTAGCGGAGCCAGATGCGAGGTAC | |
| P3109_CD8 | AACTTTTGGAGTCAGGCGGTGGACTGGTACAACCG | |
| Hinge_CD28 | GGTGGTTCATTGCGTTTGAGCTGCGCTGCCTCTGGT | |
| tm_41BB_CD3Z | CGTACGCTAAGTAGTTACGCGATGGGGTGGTACCG | |
| (nucleotide) | CCAAGCGCCGGGCAAAGAACGCGAGCTCGTTAGTA | |
| | GTATTTCCGGGAGTGGAGGCTCTACCCGTTACGCG | |
| | GACTCGGTAAAAGGCCGTTTTACGATCAGTCGTGA | |
| | TAATTCCAAGAATACCTTGTACCTGCAAATGAATA | |
| | GCCTTAAGCCCGAAGACACAGCGGTGTATTATTGT | |
| | GCCGTCTACATTCTGGAGCTGGCTCCTGGCGCGGA | |
| | ATACTGGGGCCAGGGTACCCAGGTGACGGTCTCGA | |
| | GCACAACAACTCCAGCCCCAAGACCACCTACGCCT | |
| | GCACCTACTATCGCATCTCAACCACTGTCCCTGCGC | |
| | CCTGAGGCATGCCGACCAGCAGCCGGTGGCGCGGT | |
| | GCATACCCGCGGACTGGACTTTGCCTGCGATTTTTG | |
| | GGTGCTGGTGGTGGTTGGTGGAGTCCTGGCTTGCT | |
| | ATAGCTTGCTAGTAACAGTGGCCTTTATTATTTTCT | |
| | GGGTGAAACGGGGCAGAAAGAAACTCCTGTATAT | |
| | ATTCAAACAACCATTTATGCGACCAGTACAAACTA | |
| | CTCAAGAGGAAGATGGCTGTAGCTGCCGATTTCCA | |
| | GAAGAAGAAGAAGGAGGATGTGAACTGAGAGTGA | |
| | AGTTCAGCAGGAGCGCAGACGCCCCCGCGTACCAG | |
| | CAGGGCCAGAACCAGCTCTATAACGAGCTCAATCT | |
| | AGGACGAAGAGAGGAGTACGATGTTTTGGACAAG | |
| | CGACGTGGCCGGGACCCTGAGATGGGGGGAAAGC | |
| | CGAGAAGGAAGAACCCTCAGGAAGGCCTGTACAA | |
| | TGAACTGCAGAAAGATAAGATGGCGGAGGCCTAC | |
| | AGTGAGATTGGGATGAAAGGCGAGCGCCGGAGGG | |
| | GCAAGGGGCACGATGGCCTTTACCAGGGACTCAGT | |
| | ACAGCCACCAAGGACACCTACGACGCCCTTCACAT | |
| | GCAGGCCCTGCCCCCTCGC | |
| MARS | ATGGCCAGATCTCCTGCTCAACTGCTGGGACTGCT | 184 |
| SP_NEC_M_46_ | GCTGCTGTGGCTTAGCGGAGCCAGATGCGAGGTAC | |
| P3110_CD8 | AACTTTTGGAGTCAGGCGGTGGACTGGTACAACCG | |
| Hinge_CD28 | GGTGGTTCATTGCGTTTGAGCTGCGCTGCCTCTGGT | |
| tm_41BB_CD3Z | TACACATTTTCGGATTATGCAATGGGTTGGTATCGC | |
| (nucleotide) | CAAGCGCCGGGCAAAGAACGCGAGCTCGTTAGTA | |
| | GTATTTCCGGGAGTGGAGGCTCTACCCGTTACGCG | |
| | GACTCGGTAAAAGGCCGTTTTACGATCAGTCGTGA | |
| | TAATTCCAAGAATACCTTGTACCTGCAAATGAATA | |
| | GCCTTAAGCCCGAAGACACAGCGGTGTATTATTGT | |
| | GCCGCAGTAATTCGGCAGCCTAGCACCGGTTTCTA | |
| | TGAATACTGGGGCCAGGGTACCCAGGTGACGGTCT | |
| | CGAGCACAACAACTCCAGCCCCAAGACCACCTACG | |
| | CCTGCACCTACTATCGCATCTCAACCACTGTCCCTG | |
| | CGCCCTGAGGCATGCCGACCAGCAGCCGGTGGCGC | |
| | GGTGCATACCCGCGGACTGGACTTTGCCTGCGATT | |
| | TTTGGGTGCTGGTGGTGGTTGGTGGAGTCCTGGCTT | |
| | GCTATAGCTTGCTAGTAACAGTGGCCTTTATTATTT | |
| | TCTGGGTGAAACGGGGCAGAAAGAAACTCCTGTAT | |
| | ATATTCAAACAACCATTTATGCGACCAGTACAAAC | |
| | TACTCAAGAGGAAGATGGCTGTAGCTGCCGATTTC | |
| | CAGAAGAAGAAGAAGGAGGATGTGAACTGAGAGT | |
| | GAAGTTCAGCAGGAGCGCAGACGCCCCCGCGTACC | |
| | AGCAGGGCCAGAACCAGCTCTATAACGAGCTCAAT | |
| | CTAGGACGAAGAGAGGAGTACGATGTTTTGGACAA | |
| | GCGACGTGGCCGGGACCCTGAGATGGGGGGAAAG | |
| | CCGAGAAGGAAGAACCCTCAGGAAGGCCTGTACA | |
| | ATGAACTGCAGAAAGATAAGATGGCGGAGGCCTA | |
| | CAGTGAGATTGGGATGAAAGGCGAGCGCCGGAGG | |
| | GGCAAGGGGCACGATGGCCTTTACCAGGGACTCAG | |
| | TACAGCCACCAAGGACACCTACGACGCCCTTCACA | |
| | TGCAGGCCCTGCCCCCTCGC | |

In some embodiments, the antigen-binding domain of the second polypeptide binds to an antigen. The antigen-binding domain of the second polypeptide may bind to more than one antigen or more than one epitope in an antigen. For example, the antigen-binding domain of the second polypeptide may bind to two, three, four, five, six, seven, eight or more antigens. As another example, the antigen-binding domain of the second polypeptide may bind to two, three, four, five, six, seven, eight or more epitopes in the same antigen.

The choice of antigen-binding domain may depend upon the type and number of antigens that define the surface of a target cell. For example, the antigen-binding domain may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding domain that specifically binds to an antigen (e.g., on a tumor cell). Non-limiting examples of cell surface markers that may act as targets for the antigen-binding domain in the CAR of the disclosure include those associated with tumor cells or autoimmune diseases.

In some embodiments, the antigen-binding domain binds to at least one tumor antigen or autoimmune antigen.

In some embodiments, the antigen-binding domain binds to at least one tumor antigen. In some embodiments, the antigen-binding domain binds to two or more tumor antigens. In some embodiments, the two or more tumor antigens are associated with the same tumor. In some embodiments, the two or more tumor antigens are associated with different tumors.

In some embodiments, the antigen-binding domain binds to at least one autoimmune antigen. In some embodiments, the antigen-binding domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of tumor antigen associated with glioblastoma include HER2, EGFRvIII, EGFR, CD133, PDGFRA, FGFR1, FGFR3, MET, CD70, ROBO1 and IL13Rα2. Non-limiting examples of tumor antigens associated with ovarian cancer include FOLR1, FSHR, MUC16, MUC1, Mesothelin, CA125, EpCAM, EGFR, PDGFRα, Nectin4, and B7H4. Non-limiting examples of the tumor antigens associated with cervical cancer or head and neck cancer include GD2, MUC1, Mesothelin, HER2, and EGFR. Non-limiting examples of tumor antigen associated with liver cancer include Claudin 18.2, GPC-3, EpCAM, cMET, and AFP. Non-limiting examples of tumor antigens associated with hematological malignancies include CD22, CD79, BCMA, GPRC5D, SLAM F7, CD33, CLL1, CD123, and CD70. Non-limiting examples of tumor antigens associated with bladder cancer include Nectin4 and SLITRK6. Non-limiting examples of tumor antigens associated with glioblastoma include Cd133, EGFr, CD70, and IL13Rα2. Non-limiting examples of tumor antigens associated with renal cell carcinoma include Nectin4, SLITRK6, CD70, and FOLR1. Non-limiting examples of tumor antigens associated with ovarian cancer include Nectin4, mesothelin, FSHR, and FOLR1. A non-limiting example of a tumor antigen associated with hepatocellular carcinoma includes GPC3.

Additional examples of antigens that may be targeted by the antigen-binding domain include, but are not limited to, alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, Nectin4, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In one embodiment, the antigen targeted by the antigen-binding domain is Nectin4. In one embodiment, the antigen-binding domain comprises an anti-Nectin4 VHH. In other embodiments, the antigen-binding domain comprises an anti-Nectin4 scFv. In one embodiment, the anti-Nectin4 antigen binding domain comprises the amino acid sequence set forth in one of SEQ ID NOs: 105-130, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with one of SEQ ID NOs: 105-130. In one embodiment, the anti-Nectin4 antigen binding domain comprises the amino acid sequence encoded by the polynucleotide sequence set forth in one of SEQ ID NOs: 131-156, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with one of SEQ ID NOs: 131-156.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, autoimmune antigens that may be targeted by the CAR disclosed herein include but are not limited to platelet antigens, myelin protein antigen, Sm antigens in snRNPs, islet cell antigen, Rheumatoid factor, and anticitrullinated protein. citrullinated proteins and peptides such as CCP-1, CCP-2 (cyclical citrullinated peptides), fibrinogen, fibrin, vimentin, filaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic translation elongation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB′/B), U1RNP, A2/B1 hnRNP, Ro (SSA), and La (SSB) antigens.

In various embodiments, a CAR of the present disclosure can comprise an scFv domain or fragment thereof, and the scFv domain or fragment thereof used in the CAR may include a linker between the VH and VL domains. The linker can be a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to an antigen. The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that may be used are Gly rich linkers, Gly and Ser containing linkers, Gly and Ala containing linkers, Ala and Ser containing linkers, and other flexible linkers.

In one embodiment, a CAR can comprise a linker, and the linker is a Whitlow linker. In one embodiment, the Whitlow linker comprises the amino acid sequence set forth in SEQ ID NO: 3, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 3. In another embodiment, the linker is a (G₄S)₃ linker. In one embodiment, the (G₄S)₃ linker comprises the amino acid sequence set forth in SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25.

Other linker sequences may include portions of immunoglobulin hinge area, CL or CH1 derived from any immunoglobulin heavy or light chain isotype. Exemplary linkers that may be used include any of SEQ ID NOs: 3, 25-56, 99, and 102-104 in Table 1. Additional linkers are described for example in Int. Pat. Publ. No. WO2019/060695, incorporated by reference herein in its entirety.

### II. Inhibitory Chimeric Antigen Receptor (iCAR) Expression

Inhibitory chimeric antigen receptors (iCARs) are genetically engineered receptors used in cell-based cancer therapies. They are a modification of the conventional chimeric antigen receptor (CAR) technology, which can be used to enhance the cancer-fighting abilities of immune cells. In contrast with CARs (e.g., synthetic receptors expressed on the surface of immune cells to enhance their ability to recognize and attack cancer cells, iCARs are designed to inhibit the activation of T cells when they encounter their target antigen. iCARs consist of several components, including an antigen binding domain (e.g., an extracellular domain), a signal peptide, a hinge region, a transmembrane domain, and an endodomain domain (e.g., an inhibitory domain). The inhibitory domain is usually derived from immune checkpoint molecules, such as PD-1 (programmed cell death protein 1) or CTLA-4 (cytotoxic T-lymphocyte-associated protein 4), which are known to suppress immune cell activity. By incorporating an inhibitory component, iCARs allow for a more controlled immune response against cancer cells by reducing off-target effects and the risk of toxicities associated with cell-based cancer therapies. Non-limiting exemplary iCAR regions and sequences are provided in Tables 4-6, including amino acid and nucleic acid sequences for the various iCAR constructs of the present disclosure. SP=signal peptide; H=hinge; and TMD=transmembrane domain.

TABLE 4

| | |
|---|---|
| | SEQ ID |
| Sequence | NO |

Signaling/Co-stimulatory Domains:

| | | |
|---|---|---|
| PD1 | CSRAARGTIGARRTGQPLKEDPSAVPVFSVDYGELDFQWREKTPEPPVPCVPEQTEYATIV | 267 |
| (amino acid) | FPSGMGTSSPARRGSADGPRSAQPLRPEDGHCSWPL | |
| PD1 | TGTAGTCGCGCTGCAAGAGGTACCATTGGTGCGAGGAGGACCGGACAACCTTTGAAG | 266 |
| (nucleic acid) | GAGGACCCAAGTGCGGTACCTGTTTTTTCCGTCGACTACGGCGAGCTGGATTTCCAGT | |
| | GGCGAGAAAAGACACCAGAACCACCGGTTCCTTGTGTACCTGAGCAGACGGAGTATG | |
| | CGACAATCGTCTTCCCGAGCGGAATGGGCACGAGTTCCCCCGCCAGAAGGGGGTCTG | |
| | CCGACGGACCTAGGAGCGCACAACCACTTAGACCCGAAGACGGCCATTGTTCCTGGC | |
| | CACTG | |
| LIRB 1 | LRHRRQGKHWTSTQRKADFQHPAGAVGPEPTDRGLQWRSSPAADAQEENLYAAVKHTQ | 268 |
| (amino acid) | PEDGVEMDTRSPHDEDPQAVTYAEVKHSRPRREMASPPSPLSGEFLDTKDRQAEEDRQM | |
| | DTEAAASEAPQDVTYAQLHSLTLRREATEPPPSQEGPSPAVPSIYATLAIH | |
| LIRB 1 | CTCCGACATAGACGCCAAGGCAAACACTGGACGTCTACTCAACGAAAAGCTGATTTT | 293 |
| (nucleic acid) | CAGCATCCCGCCGGCGCCGTAGGTCCTGAACCAACGGATCGGGGTTTGCAATGGCGA | |
| | AGTAGTCCTGCTGCTGATGCACAGGAGGAGAACCTTTATGCGGCTGTAAAACACACT | |
| | CAACCCGAAGATGGGGTCGAAATGGACACGCGAAGCCCCCATGACGAAGATCCCCA | |
| | GGCGGTCACATATGCAGAAGTCAAACACAGCCGCCCAAGGCGGGAAATGGCGAGTC | |
| | CCCCAAGCCCACTCTCAGGAGAGTTCCTTGATACCAAGGACCGCCAGGCCGAGGAGG | |
| | ACAGGCAGATGGATACTGAGGCTGCCGCATCAGAGGCACCACAAGATGTTACCTACG | |
| | CGCAACTGCACTCATTGACGCTTCGAAGAGAGGCCACCGAACCCCCACCAAGTCAGG | |
| | AAGGCCCGAGCCCTGCGGTCCCATCTATATACGCAACCCTGGCCATTCAT | |
| TIGIT | LTRKKKALRIHSVEGDLRRKSAGQEEWSPSAPSPPGSCVQAEAAPAGLCGEQRGEDCAEL | 269 |
| (amino acid) | HDYFNVLSYRSLGNCSFFTETG | |
| TIGIT | CTCACCCGAAAGAAGAAGGCTTTGAGGATTCATTCCGTAGAAGGCGACCTGCGACGC | 294 |
| (nucleic acid) | AAGTCAGCTGGTCAAGAGGAATGGTCACCATCCGCACCGTCTCCGCCTGGGTCATGT | |
| | GTTCAAGCTGAAGCGGCTCCTGCCGGGCTTTGTGGTGAACAACGGGGCGAAGATTGC | |
| | GCCGAGTTGCATGATTATTTCAACGTTCTTAGTTACCGCTCTCTCGGAAATTGTAGTTT | |
| | TTTTACCGAGACCGGA | |
| CTLA4 | AVSLSKMLKKRSPLTTGVYVKMPPTEPECEKQFQPYFIPIN | 270 |
| (amino acid) | | |
| CTLA4 | GCAGTCTCACTCTCCAAAATGCTGAAAAAGCGCTCTCCCCTTACCACCGGCGTCTATG | 295 |
| (nucleic acid) | TCAAAATGCCACCTACTGAACCTGAATGCGAGAAGCAGTTCCAACCTTATTTTATCCC | |
| | CATTAAT | |
| CSK*(YSSV) | YSSV | 271 |
| (amino acid) | | |
| CSK*(YSSV) | TACTCATCTGTT | 296 |
| (nucleic acid) | | |
| KIR2DL1 | HRWCSNKKNAAVMDQESAGNRTANSEDSDEQDPQEVTYTQLNHCVFTQRKITRPSQRP | 272 |
| (amino acid) | KTPPTDIIVYTELPNAESRSKVVSCP | |
| KIR2DL1 | CACCGATGGTGTTCTAACAAAAAGAACGCAGCCGTTATGGACCAGGAAAGCGCGGGA | 297 |
| (nucleic acid) | AACCGGACGGCGAACAGCGAAGACTCCGATGAGCAGGATCCGCAGGAGGTAACTTA | |
| | CACTCAGCTTAATCACTGTGTATTCACTCAAAGGAAGATCACCCGGCCTTCTCAAAGA | |
| | CCGAAAACTCCGCCTACGGACATTATTGTTTATACGGAGCTTCCAAACGCTGAGTCTC | |
| | GGAGTAAGGTGGTGTCTTGCCCA | |
| DR1 | KRKEVQKTCRKHRKENQGSHESPTLNPETVAINLSDVDLSKYITTIAGVMTLSQVKGFVR | 273 |
| (amino acid) | KNGVNEAKIDEIKNDNVQDTAEQKVQLLRNWHQLHGKKEAYDTLIKDLKKANLCTLAE | |
| | KIQTIILKDITSDSENSNFRNEIQSLV | |
| DR1 | AAGCGAAAAGAGGTGCAAAAGACGTGTAGAAAGCACCGGAAGGAGAACCAGGGGTC | 298 |
| (nucleic acid) | ACACGAGAGCCCGACACTGAACCCAGAGACGGTTGCAATAAATCTTTCTGATGTGGA | |
| | TTTGAGCAAGTATATCACAACAATCGCCGGAGTCATGACGCTGAGCCAGGTTAAGGG | |
| | CTTTGTACGCAAGAATGGCGTAAACGAGGCAAAAATTGACGAGATTAAAAATGACAA | |
| | CGTGCAGGACACCGCTGAGCAGAAGGTACAACTTTTGAGGAACTGGCATCAACTTCA | |
| | TGGAAAGAAGGAGGCGTATGACACTCTTATTAAGGACTTGAAGAAGGCCAATCTCTG | |
| | TACACTTGCAGAGAAAATACAAACGATTATCTTGAAGGATATAACTAGTGACAGTGA | |
| | GAACTCTAATTTTAGAAATGAAATCCAGTCTCTGGTC | |
| Casp8wt | MDFSRNLYDIGEQLDSEDLASLKFLSLDYIPQRKQEPIKDALMLFQRLQEKRMLEESNLSF | 274 |
| (amino acid) | LKELLFRINRLDLLITYLNTRKEEMERELQTPGRAQISAYRVMLYQISEEVSRSELRSFKFL | |
| | LQEEISKCKLDDDMNLLDIFIEMEKRVILGEGKLDILKRVCAQINKSLLKIINDYEEFSKERS | |
| | SSLEGSPDEFSNGEELCGVMTISDSPREQDSESQTLDKVYQMKSKPRGYCLIINNHNFAKA | |
| | REKVPKLHSIRDRNGTHLDAGALTTTFEELHFEIKPHDDCTVEQIYEILKIYQLMDHSNMD | |
| | CFICCILSHGDKGIIYGTDGQEAPIYELTSQFTGLKCPSLAGKPKVFFIQACQGDNYQKGIP | |
| | VETDSEEQPYLEMDLSSPQTRYIPDEADFLLGMATVNNCVSYRNPAEGTWYIQSLCQSLR | |
| | ERCPRGDDILTILTEVNYEVSNKDDKKNMGKQMPQPTFTLRKKLVFPSD* | |
| Casp8wt | ATGGACTTCAGCAGAAATTTGTATGATATCGGAGAACAACTCGATTCCGAGGATCTTG | 299 |
| (nucleic acid) | CGTCACTTAAGTTCCTCAGCCTGGACTACATACCCCAGCGCAAGCAAGAACCTATAA | |
| | AGGACGCGCTCATGTTGTTCCAACGCCTCCAAGAGAAACGCATGCTCGAAGAGTCCA | |
| | ATTTGAGTTTTCTCAAGGAACTGCTGTTTCGGATAAACCGACTGGATCTTCTTATTACT | |
| | TATCTTAATACACGCAAGGAGGAAATGGAACGGGAGCTGCAGACTCCTGGCCGGGCA | |
| | CAGATTTCTGCCTATAGGGTCATGCTGTATCAGATATCCGAAGAAGTAAGTCGATCTG | |
| | AACTTCGCTCATTTAAATTCCTGTTGCAAGAGGAAATTTCTAAGTGCAAGCTGGATGA | |
| | CGACATGAACCTCCTGGACATATTCATAGAAATGGAAAAGCGCGTCATTTTGGGCGA | |
| | AGGAAAGCTGGATATCTTGAAACGCGTCTGCGCTCAAATAAATAAATCCTTGTTGAA | |
| | GATTATTAATGACTACGAGGAGTTTTCTAAAGAACGCTCTAGCTCTCTCGAAGGTTCA | |
| | CCTGATGAGTTTTCCAACGGCGAGGAATTGTGTGGAGTAATGACAATTAGCGATTCCC | |
| | CACGGGAACAAGACAGTGAGTCCCAAACTCTCGATAAGGTCTACCAGATGAAAAGTA | |
| | AGCCCAGGGGCTATTGCTTGATTATCAATAACCACAACTTTGCTAAGGCCCGGGAAA | |
| | AAGTACCGAAACTCCACTCCATCCGGGATCGCAATGGTACTCACCTGGACGCTGGGG | |
| | CGCTTACTACCACCTTTGAAGAGCTGCATTTTGAGATAAAACCACACGACGACTGCAC | |
| | GGTTGAACAAATCTATGAGATATTGAAAATCTATCAGCTTATGGATCATTCCAATATG | |
| | GACTGTTTCATCTGCTGCATACTTAGCCACGGGGATAAAGGTATTATATACGGTACAG | |
| | ACGGTCAAGAGGCTCCGATATATGAGCTCACGTCACAATTTACTGGTCTCAAGTGCCC | |
| | TAGTCTGGCAGGGAAGCCCAAGGTGTTCTTCATTCAGGCCTGTCAGGGCGATAATTAT | |
| | CAGAAAGGGATTCCTGTGGAGACGGACAGTGAAGAGCAACCGTACCTCGAGATGGAT | |
| | TTGTCCTCTCCGCAGACGCGATATATTCCGGATGAAGCCGATTTCCTGCTTGGCATGG | |
| | CCACTGTGAACAACTGCGTCTCCTATCGGAATCCTGCAGAAGGTACGTGGTATATACA | |
| | GTCTCTCTGTCAAAGTTTGAGGGAAAGGTGCCCGCGGGGAGATGATATCTTGACCATT | |
| | TTGACGGAAGTGAACTATGAAGTGAGTAACAAAGATGACAAGAAAAACATGGGTAA | |
| | GCAGATGCCGCAACCGACTTTTACCCTGCGCAAAAAACTGGTCTTCCCTTCTGAT | |
| tCasp8 | SESQTLDKVYQMKSKPRGYCLIINNHNFAKAREKVPKLHSIRDRNGTHLDAGALTTTFEE | 275 |
| (amino acid) | LHFEIKPHDDCTVEQIYEILKIYQLMDHSNMDCFICCILSHGDKGIIYGTDGQEAPIYELTSQ | |
| | FTGLKCPSLAGKPKVFFIQACQGDNYQKGIPVETDSEEQPYLEMDLSSPQTRYIPDEADFL | |
| | LGMATVNNCVSYRNPAEGTWYIQSLCQSLRERCPRGDDILTILTEVNYEVSNKDDKKNM | |
| | GKQMPQPTFTLRKKLVFPSD | |
| tCasp8 | AGTGAAAGCCAGACGCTCGATAAAGTTTACCAGATGAAGTCCAAACCACGGGGTTAC | 300 |
| (nucleic acid) | TGTCTCATAATCAATAACCACAATTTCGCCAAGGCAAGAGAAAAAGTTCCTAAGCTC | |
| | CATAGCATCAGAGACAGGAACGGGACTCACTTGGACGCTGGGGCTCTGACTACCACT | |
| | TTCGAGGAGTTGCACTTCGAGATTAAACCACATGACGACTGCACAGTAGAGCAGATT | |
| | TACGAAATACTCAAAATCTATCAACTTATGGATCATAGCAATATGGACTGCTTTATCT | |
| | GTTGTATACTTTCCCACGGTGATAAGGGCATCATATACGGGACCGATGGTCAGGAAG | |
| | CTCCAATATATGAACTCACAAGTCAGTTTACTGGTCTGAAGTGTCCATCATTGGCGGG | |
| | GAAGCCCAAGGTCTTTTTTATTCAGGCCTGTCAAGGCGACAACTACCAAAAGGGCATT | |
| | CCAGTAGAGACAGACTCAGAGGAACAGCCATATCTCGAGATGGATCTTTCCTCCCCTC | |
| | AAACCAGATACATTCCAGATGAAGCAGACTTCCTGTTGGGGATGGCTACTGTTAATA | |
| | ACTGTGTTTCATATCGCAACCCAGCGGAGGGAACATGGTACATACAGTCCTTGTGTCA | |
| | AAGTCTGAGGGAACGATGCCCAAGGGGGGATGATATCCTGACTATCTTGACCGAGGT | |
| | GAACTACGAGGTGTCCAACAAAGACGATAAAAAGAATATGGGGAAACAAATGCCTC | |
| | AGCCAACTTTTACGCTTCGAAAAAAGCTCGTCTTCCCCAGCGAT | |
| tCasp8-dimer | SESQTLDKVYQMKSKPRGYCLIINNHNFAKAREKVPKLHSIRDRNGTHLDAGALTTTFEE | 276 |
| (amino acid) | LHFEIKPHDDCTVEQIYEILKIYQLMDHSNMDCFICCILSHGDKGIIYGTDGQEAPIYELTSQ | |
| | FTGLKCPSLAGKPKVFFIQACQGDNYQKGIPVETDSEEQPYLEMDLSSPQTRYIPDEADFL | |
| | LGMATVNNCVSYRNPAEGTWYIQSLCQSLRERCPRGDDILTILTEVNYEVSNKDDKKNM | |
| | GKQMPCIVSMLRKKLVFPSD* | |
| tCasp8-dimer | TCAGAATCACAAACTCTGGATAAGGTATATCAGATGAAGAGCAAACCGCGAGGATAT | 301 |
| (nucleic acid) | TGCTTGATTATCAACAACCATAATTTCGCTAAAGCTAGAGAAAAAGTCCCAAAACTG | |
| | CACTCCATTAGAGACAGGAATGGCACCCATTTGGACGCGGGTGCGCTCACTACCACA | |
| | TTCGAAGAATTGCACTTTGAAATTAAGCCCCACGATGACTGCACTGTGGAACAGATTT | |
| | ACGAGATACTGAAAATCTATCAACTTATGGACCACTCCAACATGGATTGTTTTATATG | |
| | CTGCATTTTGTCCCATGGTGATAAGGGAATCATATACGGAACAGATGGACAGGAAGC | |
| | GCCAATTTATGAACTTACCAGCCAGTTCACGGGACTTAAGTGTCCGAGCCTTGCTGGG | |
| | AAGCCGAAGGTCTTTTTTATACAAGCGTGTCAAGGTGACAACTATCAGAAAGGAATT | |
| | CCAGTCGAAACTGATTCTGAAGAGCAGCCATACCTGGAGATGGATCTCAGTTCTCCCC | |
| | AGACCAGGTACATTCCCGATGAAGCGGATTTTTTGCTCGGTATGGCAACAGTGAACA | |
| | ACTGTGTTTCTTACAGGAACCCGGCAGAAGGTACTTGGTATATTCAAAGCTTGTGCCA | |
| | ATCTCTTCGGGAGCGGTGTCCGAGAGGAGACGACATCCTCACTATACTCACGGAAGT | |
| | AAATTATGAGGTGAGTAACAAGGACGATAAAAAAAACATGGGAAAACAAATGCCCT | |
| | GCATAGTTTCTATGTTGAGAAAAAAACTCGTTTTTCCTAGCGAC | |
| tBid15 | GNRSSHSRLGRIEADSESQEDIIRNIARHLAQVGDSMDRSIPPGLVNGLALQLRNTSRSEED | 277 |
| (amino acid) | RNRDLATALEQLLQAYPRDMEKEKTMLVLALLLAKKVASHTPSLLRDVFHTTVNFINQN | |
| | LRTYVRSLARNGMD | |
| tBid15 | GGCAATCGAAGTTCTCACTCACGCCTTGGACGGATCGAAGCAGATTCAGAAAGTCAA | 302 |
| (nucleic acid) | GAGGACATCATACGGAACATAGCCCGGCATTTGGCCCAAGTAGGGGATAGTATGGAC | |
| | CGGAGTATTCCACCTGGTCTTGTAAACGGTTTGGCTTTGCAGTTGAGAAACACCAGTA | |
| | GGAGTGAAGAAGACCGCAACAGGGATCTCGCCACGGCCCTTGAGCAACTTTTGCAAG | |
| | CATATCCAAGAGATATGGAAAAAGAAAAGACGATGTTGGTCCTTGCATTGCTTCTGG | |
| | CAAAAAAGGTTGCGTCTCACACGCCTTCTTTGTTGCGCGATGTCTTTCACACTACGGT | |
| | AAATTTCATCAACCAGAACCTTCGGACTTACGTGCGGTCTCTTGCCCGAAACGGCATG | |
| | GAT | |
| Casp9wt | MDEADRRLLRRCRLRLVEELQVDQLWDALLSRELFRPHMIEDIQRAGSGSRRDQARQLII | 278 |
| (amino acid) | DLETRGSQALPLFISCLEDTGQDMLASFLRTNRQAAKLSKPTLENLTPVVLRPEIRKPEVL | |
| | RPETPRPVDIGSGGFGDVGALESLRGNADLAYILSMEPCGHCLIINNVNFCRESGLRTRTG | |
| | SNIDCEKLRRRFSSLHFMVEVKGDLTAKKMVLALLELAQQDHGALDCCVVVILSHGCQA | |
| | SHLQFPGAVYGTDGCPVSVEKIVNIFNGTSCPSLGGKPKLFFIQACGGEQKDHGFEVASTS | |
| | PEDESPGSNPEPDATPFQEGLRTFDQLDAISSLPTPSDIFVSYSTFPGFVSWRDPKSGSWYV | |
| | ETLDDIFEQWAHSEDLQSLLLRVANAVSVKGIYKQMPGCFNFLRKKLFFKTS | |
| Casp9wt | ATGGACGAGGCTGATCGCAGATTGCTTAGGCGATGTCGGTTGAGACTGGTAGAAGAG | 303 |
| (nucleic acid) | TTGCAAGTCGATCAATTGTGGGATGCGCTTCTGTCTAGGGAGCTGTTTCGGCCGCACA | |
| | TGATCGAAGACATCCAAAGGGCAGGTTCAGGATCACGCCGAGATCAAGCGAGACAG | |
| | CTGATAATCGATCTGGAAACGCGAGGATCACAGGCGTTGCCTCTCTTCATCAGTTGTC | |
| | TGGAGGATACGGGGCAGGACATGCTCGCTTCCTTTCTGCGAACCAATCGCCAAGCCG | |
| | CAAAGCTCAGCAAACCGACACTTGAAAACCTGACGCCGGTTGTGCTGAGGCCAGAAA | |
| | TACGGAAACCAGAGGTCCTGAGACCCGAAACACCACGACCAGTAGACATTGGGAGC | |
| | GGGGGTTTCGGAGATGTAGGCGCATTGGAGTCCCTTCGAGGCAATGCGGATCTTGCA | |
| | TATATTCTTAGTATGGAACCTTGTGGACACTGTCTCATTATCAACAATGTGAATTTCTG | |
| | CCGCGAGTCAGGACTGCGAACCCGGACAGGCTCAAACATTGATTGTGAGAAACTTCG | |
| | ACGCAGATTCTCATCCTTGCATTTTATGGTAGAGGTTAAGGGGGATCTTACAGCGAAG | |
| | AAAATGGTTCTTGCTCTGCTCGAACTTGCACAGCAAGACCACGGGGCACTGGATTGTT | |
| | GCGTAGTCGTGATTCTTTCCCATGGATGTCAAGCGTCTCATCTCCAGTTTCCGGGCGC | |
| | GGTCTATGGTACGGACGGCTGCCCCGTTTCAGTAGAAAAGATAGTCAACATTTTCAAC | |
| | GGCACCTCATGCCCGTCCTTGGGTGGCAAACCCAAGTTGTTTTTTATCCAAGCGTGCG | |
| | GTGGAGAACAGAAAGATCATGGGTTTGAAGTGGCGTCCACATCACCCGAAGATGAAA | |
| | GCCCTGGGAGCAACCCTGAACCAGACGCGACTCCTTTTCAAGAGGGCTTGCGGACTTT | |
| | TGACCAGTTGGACGCCATATCATCATTGCCGACGCCGTCTGATATATTCGTCTCCTAT | |
| | AGTACATTCCCTGGTTTTGTCTCTTGGAGGGACCCCAAAAGCGGCTCTTGGTATGTTG | |
| | AAACATTGGATGATATTTTTGAACAATGGGCGCACAGTGAGGATCTTCAGAGCTTGCT | |
| | TCTGCGAGTCGCTAACGCAGTTTCCGTAAAGGGAATATACAAACAGATGCCCGGATG | |
| | CTTTAATTTCCTCCGCAAAAAGCTGTTTTTTAAGACTTCT | |
| tCasp9 | GFGDVGALESLRGNADLAYILSMEPCGHCLIINNVNFCRESGLRTRTGSNIDCEKLRRRFS | 279 |
| (amino acid) | SLHFMVEVKGDLTAKKMVLALLELARQDHGALDCCVVVILSHGCQASHLQFPGAVYGT | |
| | DGCPVSVEKIVNIFNGTSCPSLGGKPKLFFIQACGGEQKDHGFEVASTSPEDESPGSNPEPD | |
| | ATPFQEGLRTFDQLDAISSLPTPSDIFVSYSTFPGFVSWRDPKSGSWYVETLDDIFEQWAHS | |
| | EDLQSLLLRVANAVSVKGIYKQMPGCFNFLRKKLFFKTS | |
| tCasp9 | GGATTTGGGGATGTAGGAGCACTCGAATCTCTTAGGGGGAATGCTGATTTGGCTTATA | 304 |
| (nucleic acid) | TCCTTTCTATGGAACCATGCGGACACTGTCTGATAATAAATAATGTCAATTTCTGCCG | |
| | GGAAAGCGGATTGCGAACCCGAACGGGCTCAAACATCGATTGTGAGAAGCTGCGACG | |
| | ACGGTTTAGTTCCCTTCATTTTATGGTCGAAGTCAAAGGCGATCTTACCGCCAAGAAA | |
| | ATGGTTTTGGCACTCTTGGAGCTTGCGCGACAAGATCACGGGGCATTGGATTGTTGTG | |
| | TTGTGGTCATCTTGAGCCATGGTTGTCAAGCGTCACACTTGCAGTTTCCCGGTGCCGTT | |
| | TATGGGACTGATGGCTGTCCAGTATCCGTTGAAAAGATCGTCAATATATTTAATGGCA | |
| | CATCTTGTCCTTCCCTGGGCGGGAAGCCCAAACTCTTTTTTATTCAGGCCTGCGGTGGT | |
| | GAACAGAAAGATCACGGTTTTGAAGTTGCATCTACCTCTCCAGAAGACGAATCCCCT | |
| | GGGAGCAACCCTGAACCTGACGCGACTCCATTCCAAGAGGGGCTTCGGACGTTCGAC | |
| | CAACTCGACGCAATATCAAGTTTGCCGACACCGAGCGACATATTCGTCTCATACAGCA | |
| | CATTCCCCGGTTTCGTATCTTGGAGAGATCCAAAGTCAGGGAGTTGGTATGTCGAGAC | |
| | TTTGGATGATATTTTTGAACAATGGGCGCACTCCGAGGATCTTCAGAGTCTTCTCCTG | |
| | CGGGTGGCGAATGCGGTGAGCGTGAAAGGAATTTACAAGCAGATGCCAGGGTGCTTT | |
| | AACTTCCTCCGGAAAAAGCTGTTTTTCAAAACTAGC | |
| tCasp9-dimer | GFGDVGALESLRGNADLAYILSMEPCGHCLIINNVNFCRESGLRTRTGSNIDCEKLRRRFS | 280 |
| (amino acid) | SLHFMVEVKGDLTAKKMVLALLELAQQDHGALDCCVVVILSHGCQASHLQFPGAVYGT | |
| | DGCPVSVEKIVNIFNGTSCPSLGGKPKLFFIQACGGEQKDHGFEVASTSPEDESPGSNPEPD | |
| | ATPFQEGLRTFDQLDAISSLPTPSDIFVSYSTFPGFVSWRDPKSGSWYVETLDDIFEQWAHS | |
| | EDLQSLLLRVANAVSVKGIYKQMPCIVSMLRKKLFFKTS | |
| tCasp9-dimer | GGATTTGGTGATGTCGGTGCCCTCGAAAGTCTTAGAGGCAACGCTGATCTGGCGTATA | 305 |
| (nucleic acid) | TTTTGAGCATGGAACCATGTGGACATTGTCTTATCATCAATAACGTAAATTTTTGTAG | |
| | GGAAAGTGGCCTTAGGACTAGGACAGGTTCCAACATCGACTGCGAAAAACTGCGGAG | |
| | GAGATTCTCTTCCCTCCATTTTATGGTGGAAGTCAAAGGCGATCTCACCGCAAAGAAG | |
| | ATGGTACTCGCATTGCTCGAACTTGCCCAACAGGACCATGGAGCTTTGGACTGCTGTG | |
| | TTGTAGTTATACTGTCACATGGTTGCCAGGCAAGCCACCTCCAGTTTCCTGGGGCGGT | |
| | ATATGGGACCGATGGTTGCCCCGTTTCCGTAGAGAAGATAGTAAATATCTTCAACGG | |
| | AACGAGCTGTCCGTCCCTCGGGGGCAAGCCAAAGCTTTTTTTCATTCAAGCCTGTGGC | |
| | GGCGAGCAAAAAGACCACGGATTCGAGGTGGCATCAACGTCCCCTGAAGATGAGAGT | |
| | CCGGGCAGCAATCCCGAACCGGATGCTACCCCATTCCAAGAAGGTCTTAGGACATTC | |
| | GACCAACTGGACGCTATATCTAGTTTGCCAACTCCTTCAGATATCTTTGTAAGTTACTC | |
| | TACGTTTCCTGGCTTCGTAAGTTGGAGAGATCCTAAATCTGGAAGTTGGTATGTGGAA | |
| | ACGTTGGATGACATTTTTGAACAATGGGCTCATAGTGAAGACCTTCAGTCACTCCTGC | |
| | TTCGCGTGGCAAATGCAGTGTCAGTTAAGGGTATCTACAAACAGATGCCGTGCATCGT | |
| | ATCCATGCTTCGGAAAAAGCTGTTCTTTAAAACAAGC | |
| SHP1 | MVRWFHRDLSGLDAETLLKGRGVHGSFLARPSRKNQGDFSLSVRVGDQVTHIRIQNSGD | 281 |
| (amino acid) | FYDLYGGEKFATLTELVEYYTQQQGVLQDRDGTIIHLKYPLNCSDPTSERWYHGHMSGG | |
| | QAETLLQAKGEPWTFLVRESLSQPGDFVLSVLSDQPKAGPGSPLRVTHIKVMCEGGRYTV | |
| | GGLETFDSLTDLVEHFKKTGIEEASGAFVYLRQPYYATRVNAADIENRVLELNKKQESED | |
| | TAKAGFWEEFESLQKQEVKNLHQRLEGQRPENKGKNRYKNILPFDHSRVILQGRDSNIPG | |
| | SDYINANYIKNQLLGPDENAKTYIASQGCLEATVNDFWQMAWQENSRVIVMTTREVEKG | |
| | RNKCVPYWPEVGMQRAYGPYSVTNCGEHDTTEYKLRTLQVSPLDNGDLIREIWHYQYLS | |
| | WPDHGVPSEPGGVLSFLDQINQRQESLPHAGPIIVHCSAGIGRTGTIIVIDMLMENISTKGL | |
| | DCDIDIQKTIQMVRAQRSGMVQTEAQYKFIYVAIAQFIETTKKKLEVLQSQKGQESEYGNI | |
| | TYPPAMKNAHAKASRTSSKHKEDVYENLHTKNKREEKVKKQRSADKEKSKGSLKRK | |
| SHP1 | ATGGTACGATGGTTCCATCGCGACTTGTCCGGACTGGACGCTGAGACCCTCCTGAAAG | 306 |
| (nucleic acid) | GGCGGGGTGTGCACGGCAGTTTCCTTGCACGACCCTCCAGAAAGAATCAAGGCGACT | |
| | TTAGTCTTTCAGTAAGGGTTGGTGATCAGGTCACACACATTAGAATCCAAAATTCAGG | |
| | TGACTTCTATGATTTGTATGGCGGTGAGAAATTTGCAACGCTCACCGAACTTGTTGAA | |
| | TATTACACCCAGCAACAGGGAGTACTTCAGGACCGCGACGGTACAATAATTCACCTT | |
| | AAGTACCCCTTGAATTGTTCAGATCCTACGTCCGAGAGATGGTATCACGGACACATGA | |
| | GTGGAGGACAAGCTGAAACCCTCCTCCAAGCAAAGGGGGAACCCTGGACTTTTTTGG | |
| | TCAGAGAGAGTCTTAGCCAGCCGGGTGATTTTGTTCTTAGTGTGCTGTCCGATCAACC | |
| | GAAAGCGGGGCCTGGTTCACCTTTGCGCGTGACCCATATTAAAGTTATGTGTGAAGG | |
| | AGGTCGATATACCGTTGGGGGCCTTGAAACATTCGATAGTCTGACGGATTTGGTCGAA | |
| | CACTTCAAAAAAACAGGGATTGAAGAGGCCTCCGGCGCTTTTGTCTATCTTAGACAAC | |
| | CCTATTATGCGACGAGGGTGAACGCTGCGGATATCGAGAATAGAGTTCTGGAGCTGA | |
| | ATAAAAAGCAAGAATCAGAGGACACCGCCAAAGCTGGTTTCTGGGAGGAGTTCGAG | |
| | AGTTTGCAGAAGCAGGAAGTTAAAAACCTCCACCAGAGGTTGGAAGGGCAGAGACC | |
| | GGAAAACAAGGGTAAGAACCGCTATAAAAATATCTTGCCCTTCGATCACTCTCGGGT | |
| | CATACTGCAGGGTAGAGACAGTAACATTCCAGGCAGTGATTACATCAACGCTAACTA | |
| | TATAAAAAATCAGCTTCTGGGCCCAGATGAAAATGCAAAGACCTATATTGCGAGTCA | |
| | GGGCTGTCTGGAGGCCACGGTTAATGACTTCTGGCAGATGGCATGGCAAGAAAACAG | |
| | TAGGGTAATCGTCATGACAACTAGAGAAGTTGAGAAAGGACGGAACAAGTGTGTTCC | |
| | TTACTGGCCCGAGGTAGGCATGCAGCGGGCGTACGGGCCCTACAGTGTTACCAACTG | |
| | CGGCGAACATGATACCACAGAGTATAAATTGCGAACACTCCAAGTGTCACCACTGGA | |
| | TAACGGCGACCTTATCAGGGAGATCTGGCACTACCAATACCTGTCCTGGCCTGATCAC | |
| | GGCGTACCATCCGAACCGGGGGGAGTGCTTAGCTTTCTGGATCAGATAAACCAAAGA | |
| | CAGGAATCTCTGCCCCATGCAGGTCCGATCATCGTCCATTGTTCCGCGGGTATAGGTC | |
| | GGACCGGAACGATTATCGTAATTGACATGTTGATGGAAAACATCTCTACGAAAGGGC | |
| | TCGATTGCGATATAGACATCCAGAAGACCATACAAATGGTACGGGCACAAAGATCAG | |
| | GCATGGTCCAGACCGAAGCCCAATACAAATTCATCTACGTCGCTATTGCCCAGTTCAT | |
| | TGAAACAACGAAAAAGAAGTTGGAGGTTCTCCAATCCCAGAAGGGACAAGAGTCTG | |
| | AGTACGGTAACATAACTTATCCGCCTGCCATGAAAAACGCTCATGCAAAGGCGAGCC | |
| | GGACTAGTAGTAAGCACAAAGAAGACGTTTACGAGAATCTGCATACCAAAAATAAGC | |
| | GGGAGGAAAAAGTAAAAAAACAACGATCAGCTGATAAAGAGAAATCTAAAGGCTCA | |
| | TTGAAGAGAAAA | |
| (G4S)2-SHP1 | GGGGSGGGGSMVRWFHRDLSGLDAETLLKGRGVHGSFLARPSRKNQGDFSLSVRVGDQ | 282 |
| (amino acid) | VTHIRIQNSGDFYDLYGGEKFATLTELVEYYTQQQGVLQDRDGTIIHLKYPLNCSDPTSER | |
| | WYHGHMSGGQAETLLQAKGEPWTFLVRESLSQPGDFVLSVLSDQPKAGPGSPLRVTHIK | |
| | VMCEGGRYTVGGLETFDSLTDLVEHFKKTGIEEASGAFVYLRQPYYATRVNAADIENRV | |
| | LELNKKQESEDTAKAGFWEEFESLQKQEVKNLHQRLEGQRPENKGKNRYKNILPFDHSR | |
| | VILQGRDSNIPGSDYINANYIKNQLLGPDENAKTYIASQGCLEATVNDFWQMAWQENSR | |
| | VIVMTTREVEKGRNKCVPYWPEVGMQRAYGPYSVTNCGEHDTTEYKLRTLQVSPLDNG | |
| | DLIREIWHYQYLSWPDHGVPSEPGGVLSFLDQINQRQESLPHAGPIIVHCSAGIGRTGTIIVI | |
| | DMLMENISTKGLDCDIDIQKTIQMVRAQRSGMVQTEAQYKFIYVAIAQFIETTKKKLEVL | |
| | QSQKGQESEYGNITYPPAMKNAHAKASRTSSKHKEDVYENLHTKNKREEKVKKQRSAD | |
| | KEKSKGSLKRK | |
| (G4S)2-SHP1 | GGAGGTGGTGGGTCAGGTGGAGGCGGAAGTATGGTACGATGGTTCCATCGCGACTTG | 307 |
| (nucleic acid) | TCCGGACTGGACGCTGAGACCCTCCTGAAAGGGGGGGTGTGCACGGCAGTTTCCTT | |
| | GCACGACCCTCCAGAAAGAATCAAGGCGACTTTAGTCTTTCAGTAAGGGTTGGTGAT | |
| | CAGGTCACACACATTAGAATCCAAAATTCAGGTGACTTCTATGATTTGTATGGCGGTG | |
| | AGAAATTTGCAACGCTCACCGAACTTGTTGAATATTACACCCAGCAACAGGGAGTAC | |
| | TTCAGGACCGCGACGGTACAATAATTCACCTTAAGTACCCCTTGAATTGTTCAGATCC | |
| | TACGTCCGAGAGATGGTATCACGGACACATGAGTGGAGGACAAGCTGAAACCCTCCT | |
| | CCAAGCAAAGGGGGAACCCTGGACTTTTTTGGTCAGAGAGAGTCTTAGCCAGCCGGG | |
| | TGATTTTGTTCTTAGTGTGCTGTCCGATCAACCGAAAGCGGGGCCTGGTTCACCTTTG | |
| | CGCGTGACCCATATTAAAGTTATGTGTGAAGGAGGTCGATATACCGTTGGGGGCCTTG | |
| | AAACATTCGATAGTCTGACGGATTTGGTCGAACACTTCAAAAAAACAGGGATTGAAG | |
| | AGGCCTCCGGCGCTTTTGTCTATCTTAGACAACCCTATTATGCGACGAGGGTGAACGC | |
| | TGCGGATATCGAGAATAGAGTTCTGGAGCTGAATAAAAAGCAAGAATCAGAGGACA | |
| | CCGCCAAAGCTGGTTTCTGGGAGGAGTTCGAGAGTTTGCAGAAGCAGGAAGTTAAAA | |
| | ACCTCCACCAGAGGTTGGAAGGGCAGAGACCGGAAAACAAGGGTAAGAACCGCTAT | |
| | AAAAATATCTTGCCCTTCGATCACTCTCGGGTCATACTGCAGGGTAGAGACAGTAACA | |
| | TTCCAGGCAGTGATTACATCAACGCTAACTATATAAAAAATCAGCTTCTGGGCCCAGA | |
| | TGAAAATGCAAAGACCTATATTGCGAGTCAGGGCTGTCTGGAGGCCACGGTTAATGA | |
| | CTTCTGGCAGATGGCATGGCAAGAAAACAGTAGGGTAATCGTCATGACAACTAGAGA | |
| | AGTTGAGAAAGGACGGAACAAGTGTGTTCCTTACTGGCCCGAGGTAGGCATGCAGCG | |
| | GGCGTACGGGCCCTACAGTGTTACCAACTGCGGCGAACATGATACCACAGAGTATAA | |
| | ATTGCGAACACTCCAAGTGTCACCACTGGATAACGGCGACCTTATCAGGGAGATCTG | |
| | GCACTACCAATACCTGTCCTGGCCTGATCACGGCGTACCATCCGAACCGGGGGGAGT | |
| | GCTTAGCTTTCTGGATCAGATAAACCAAAGACAGGAATCTCTGCCCCATGCAGGTCCG | |
| | ATCATCGTCCATTGTTCCGCGGGTATAGGTCGGACCGGAACGATTATCGTAATTGACA | |
| | TGTTGATGGAAAACATCTCTACGAAAGGGCTCGATTGCGATATAGACATCCAGAAGA | |
| | CCATACAAATGGTACGGGCACAAAGATCAGGCATGGTCCAGACCGAAGCCCAATACA | |
| | AATTCATCTACGTCGCTATTGCCCAGTTCATTGAAACAACGAAAAAGAAGTTGGAGG | |
| | TTCTCCAATCCCAGAAGGGACAAGAGTCTGAGTACGGTAACATAACTTATCCGCCTGC | |
| | CATGAAAAACGCTCATGCAAAGGCGAGCCGGACTAGTAGTAAGCACAAAGAAGACG | |
| | TTTACGAGAATCTGCATACCAAAAATAAGCGGGAGGAAAAAGTAAAAAAACAACGA | |
| | TCAGCTGATAAAGAGAAATCTAAAGGCTCATTGAAGAGAAAA | |
| CSK | MSAIQAAWPSGTECIAKYNFHGTAEQDLPFCKGDVLTIVAVTKDPNWYKAKNKVGREGI | 283 |
| (amino acid) | IPANYVQKREGVKAGTKLSLMPWFHGKITREQAERLLYPPETGLFLVRESTNYPGDYTLC | |
| | VSCDGKVEHYRIMYHASKLSIDEEVYFENLMQLVEHYTSDADGLCTRLIKPKVMEGTVA | |
| | AQDEFYRSGWALNMKELKLLQTIGKGEFGDVMLGDYRGNKVAVKCIKNDATAQAFLAE | |
| | ASVMTQLRHSNLVQLLGVIVEEKGGLYIVTEYMAKGSLVDYLRSRGRSVLGGDCLLKFS | |
| | LDVCEAMEYLEGNNFVHRDLAARNVLVSEDNVAKVSDFGLTKEASSTQDTGKLPVKWT | |
| | APEALREKKFSTKSDVWSFGILLWEIYSFGRVPYPRIPLKDVVPRVEKGYKMDAPDGCPP | |
| | AVYEVMKNCWHLDAAMRPSFLQLREQLEHIKTHELHL | |
| CSK | ATGTCCGCCATACAGGCAGCATGGCCCAGCGGAACGGAATGTATCGCTAAATACAAC | 308 |
| (nucleic acid) | TTTCACGGCACCGCGGAGCAAGATCTTCCGTTCTGTAAAGGTGACGTTCTTACCATAG | |
| | TGGCGGTTACTAAGGACCCAAATTGGTATAAAGCTAAGAACAAGGTCGGGCGCGAGG | |
| | GCATAATCCCAGCAAACTATGTCCAGAAGAGGGAAGGCGTGAAAGCGGGAACCAAA | |
| | TTGAGCCTCATGCCGTGGTTCCACGGCAAGATTACACGCGAACAAGCGGAGCGACTG | |
| | CTTTATCCACCAGAGACGGGTCTTTTCCTGGTCCGGGAGTCCACGAATTATCCGGGGG | |
| | ACTATACACTTTGTGTAAGCTGTGACGGCAAGGTCGAACACTACAGGATCATGTACC | |
| | ACGCTAGTAAACTTAGCATAGATGAGGAGGTATATTTTGAGAATCTCATGCAGCTTGT | |
| | AGAACATTACACAAGTGACGCGGATGGATTGTGTACTCGATTGATTAAGCCAAAGGT | |
| | TATGGAAGGTACTGTCGCAGCCCAAGACGAGTTTTACCGATCCGGCTGGGCTCTGAA | |
| | CATGAAAGAGCTCAAACTTCTCCAGACCATCGGTAAGGGTGAGTTTGGCGACGTAAT | |
| | GTTGGGTGATTACCGCGGAAATAAGGTGGCTGTAAAATGTATCAAGAACGATGCAAC | |
| | AGCTCAAGCCTTCCTGGCTGAGGCGTCAGTGATGACACAACTTAGACACTCAAACTTG | |
| | GTCCAATTGCTCGGAGTTATCGTCGAAGAGAAAGGCGGCCTCTATATTGTTACTGAGT | |
| | ATATGGCTAAAGGCTCCCTTGTGGACTATCTTCGATCTCGAGGCAGGTCTGTTCTCGG | |
| | TGGGGATTGCTTGTTGAAATTCAGTCTTGATGTTTGCGAAGCTATGGAATATCTGGAA | |
| | GGCAACAATTTTGTCCATAGGGACCTGGCCGCCCGGAATGTGTTGGTTTCAGAAGATA | |
| | ACGTGGCCAAGGTGTCCGACTTTGGTCTGACAAAAGAGGCTAGCTCCACTCAGGACA | |
| | CTGGGAAGTTGCCGGTAAAATGGACGGCCCCTGAAGCATTGAGAGAGAAAAAATTCT | |
| | CTACTAAGTCCGACGTGTGGTCTTTTGGCATTCTTCTTTGGGAAATCTACTCATTCGGA | |
| | AGGGTCCCGTACCCTCGCATTCCGCTTAAGGACGTTGTCCCTCGGGTCGAAAAGGGCT | |
| | ACAAGATGGACGCTCCTGACGGATGCCCGCCTGCGGTCTACGAGGTAATGAAAAATT | |
| | GCTGGCATCTTGATGCTGCAATGCGCCCAAGTTTCTTGCAATTGCGAGAGCAACTGGA | |
| | ACACATAAAGACCCACGAGCTCCATCTC | |
| (G4S)2-CSK | GGGGSGGGGSMSAIQAAWPSGTECIAKYNFHGTAEQDLPFCKGDVLTIVAVTKDPNWYK | 284 |
| (amino acid) | AKNKVGREGIIPANYVQKREGVKAGTKLSLMPWFHGKITREQAERLLYPPETGLFLVRES | |
| | TNYPGDYTLCVSCDGKVEHYRIMYHASKLSIDEEVYFENLMQLVEHYTSDADGLCTRLIK | |
| | PKVMEGTVAAQDEFYRSGWALNMKELKLLQTIGKGEFGDVMLGDYRGNKVAVKCIKN | |
| | DATAQAFLAEASVMTQLRHSNLVQLLGVIVEEKGGLYIVTEYMAKGSLVDYLRSRGRSV | |
| | LGGDCLLKFSLDVCEAMEYLEGNNFVHRDLAARNVLVSEDNVAKVSDFGLTKEASSTQD | |
| | TGKLPVKWTAPEALREKKFSTKSDVWSFGILLWEIYSFGRVPYPRIPLKDVVPRVEKGYK | |
| | MDAPDGCPPAVYEVMKNCWHLDAAMRPSFLQLREQLEHIKTHELHL | |
| (G4S)2-CSK | GGAGGTGGTGGGTCAGGTGGAGGCGGAAGTATGTCCGCCATACAGGCAGCATGGCCC | 309 |
| (nucleic acid) | AGCGGAACGGAATGTATCGCTAAATACAACTTTCACGGCACCGCGGAGCAAGATCTT | |
| | CCGTTCTGTAAAGGTGACGTTCTTACCATAGTGGCGGTTACTAAGGACCCAAATTGGT | |
| | ATAAAGCTAAGAACAAGGTCGGGCGCGAGGGCATAATCCCAGCAAACTATGTCCAGA | |
| | AGAGGGAAGGCGTGAAAGCGGGAACCAAATTGAGCCTCATGCCGTGGTTCCACGGCA | |
| | AGATTACACGCGAACAAGCGGAGCGACTGCTTTATCCACCAGAGACGGGTCTTTTCCT | |
| | GGTCCGGGAGTCCACGAATTATCCGGGGGACTATACACTTTGTGTAAGCTGTGACGG | |
| | CAAGGTCGAACACTACAGGATCATGTACCACGCTAGTAAACTTAGCATAGATGAGGA | |
| | GGTATATTTTGAGAATCTCATGCAGCTTGTAGAACATTACACAAGTGACGCGGATGG | |
| | ATTGTGTACTCGATTGATTAAGCCAAAGGTTATGGAAGGTACTGTCGCAGCCCAAGA | |
| | CGAGTTTTACCGATCCGGCTGGGCTCTGAACATGAAAGAGCTCAAACTTCTCCAGACC | |
| | ATCGGTAAGGGTGAGTTTGGCGACGTAATGTTGGGTGATTACCGCGGAAATAAGGTG | |
| | GCTGTAAAATGTATCAAGAACGATGCAACAGCTCAAGCCTTCCTGGCTGAGGCGTCA | |
| | GTGATGACACAACTTAGACACTCAAACTTGGTCCAATTGCTCGGAGTTATCGTCGAAG | |
| | AGAAAGGCGGCCTCTATATTGTTACTGAGTATATGGCTAAAGGCTCCCTTGTGGACTA | |
| | TCTTCGATCTCGAGGCAGGTCTGTTCTCGGTGGGGATTGCTTGTTGAAATTCAGTCTTG | |
| | ATGTTTGCGAAGCTATGGAATATCTGGAAGGCAACAATTTTGTCCATAGGGACCTGGC | |
| | CGCCCGGAATGTGTTGGTTTCAGAAGATAACGTGGCCAAGGTGTCCGACTTTGGTCTG | |
| | ACAAAAGAGGCTAGCTCCACTCAGGACACTGGGAAGTTGCCGGTAAAATGGACGGCC | |
| | CCTGAAGCATTGAGAGAGAAAAAATTCTCTACTAAGTCCGACGTGTGGTCTTTTGGCA | |
| | TTCTTCTTTGGGAAATCTACTCATTCGGAAGGGTCCCGTACCCTCGCATTCCGCTTAAG | |
| | GACGTTGTCCCTCGGGTCGAAAAGGGCTACAAGATGGACGCTCCTGACGGATGCCCG | |
| | CCTGCGGTCTACGAGGTAATGAAAAATTGCTGGCATCTTGATGCTGCAATGCGCCCAA | |
| | GTTTCTTGCAATTGCGAGAGCAACTGGAACACATAAAGACCCACGAGCTCCATCTC | |
| ADAM17 | ITQGLAVSTISSF | 285 |
| Cleavage Site | | |
| (amino acid) | | |
| ADAM17 | ATCACCCAGGGCCTGGCCGTGAGCACCATCAGCAGCTTC | 310 |
| Cleavage Site | | |
| (nucleic acid) | | |
| 41BB | KRGRKKLLYIFKQPFMRPVQTTQEEDGCSCRFPEEEEGGCEL | 8 |
| (AA 214-255) | | |
| 41BB | AAACGGGGCAGAAAGAAACTCCTGTATATATTCAAACAACCATTTATGCGACCAGTA | 311 |
| (nucleic acid) | CAAACTACTCAAGAGGAAGATGGCTGTAGCTGCCGATTTCCAGAAGAAGAAGAAGG | |
| | AGGATGTGAACTG | |
| CD3z | RVKFSRSADAPAYQQGQNQLYNELNLGRREEYDVLDKRRGRDPEMGGKPRRKNPQEGL | 6 |
| (amino acid) | YNELQKDKMAEAYSEIGMKGERRRGKGHDGLYQGLSTATKDTYDALHMQALPPR* | |
| CD3z | AGAGTGAAGTTCAGCAGGAGCGCAGACGCCCCCGCGTACCAGCAGGGCCAGAACCA | 312 |
| (nucleic acid) | GCTCTATAACGAGCTCAATCTAGGACGAAGAGAGGAGTACGATGTTTTGGACAAGCG | |
| | ACGTGGCCGGGACCCTGAGATGGGGGGAAAGCCGAGAAGGAAGAACCCTCAGGAAG | |
| | GCCTGTACAATGAACTGCAGAAAGATAAGATGGCGGAGGCCTACAGTGAGATTGGGA | |
| | TGAAAGGCGAGCGCCGGAGGGGCAAGGGGCACGATGGCCTTTACCAGGGACTCAGT | |
| | ACAGCCACCAAGGACACCTACGACGCCCTTCACATGCAGGCCCTGCCCCCTCGCTAA | |
| ADAM17 | NTCKLLVVADHRFYRYMGRGEESTTTNYLIELIDRVDDIYRNTSWDNAGFKGYGIQIEQI | 286 |
| Protease | RILKSPQEVKPGEKHYNMAKSYPNEEKDAWDVKMLLEQFSFDIAEEASKVCLAHLFTYQ | |
| Domain | DFDMGTLGLAYVGSPRANSHGGVCPKAYYSPVGKKNIYLNSGLTSTKNYGKTILTKEAD | |
| (amino acid) | LVTTHELGHNFGAEHDPDGLAECAPNEDQGGKYVMYPIAVSGDHENNKMFSNCSKQSIY | |
| | KTIESKAQECFQERS | |
| ADAM17 | AATACGTGTAAGCTCCTTGTGGTAGCAGACCATAGGTTCTACAGATATATGGGACGA | 313 |
| Protease | GGAGAGGAAAGCACTACGACAAACTATTTGATCGAACTTATAGATAGAGTCGACGAC | |
| Domain | ATCTACCGAAACACGTCATGGGACAATGCAGGCTTCAAGGGATACGGAATACAGATA | |
| (nucleic acid) | GAGCAGATACGGATTCTGAAAAGTCCGCAGGAAGTGAAACCGGGGGAGAAACATTA | |
| | TAATATGGCGAAATCATATCCAAACGAGGAGAAAGATGCGTGGGATGTAAAAATGCT | |
| | CTTGGAACAGTTTAGTTTTGACATTGCTGAGGAAGCCAGCAAAGTGTGCCTCGCTCAC | |
| | CTGTTTACTTATCAAGATTTTGATATGGGCACCCTGGGTCTTGCATATGTAGGGTCTCC | |
| | CAGAGCAAACTCCCACGGGGGCGTATGTCCAAAAGCTTACTACAGCCCAGTAGGAAA | |
| | AAAGAATATATACCTGAACAGCGGGCTGACTAGTACCAAAAATTACGGAAAAACTAT | |
| | TCTCACGAAGGAGGCCGACTTGGTAACTACTCATGAACTGGGGCATAACTTCGGCGC | |
| | CGAGCACGACCCGGACGGACTTGCAGAGTGCGCCCCGAACGAGGACCAAGGTGGCA | |
| | AATATGTCATGTACCCAATCGCCGTTTCAGGGGATCATGAGAACAATAAGATGTTCA | |
| | GCAACTGTTCCAAACAATCCATTTATAAAACGATAGAAAGCAAAGCCCAGGAATGTT | |
| | TTCAGGAAAGATCA | |
| (G4S)3- | GGGGSGGGGSGGGGSNTCKLLVVADHRFYRYMGRGEESTTTNYLIELIDRVDDIYRNTS | 287 |
| ADAM17 | WDNAGFKGYGIQIEQIRILKSPQEVKPGEKHYNMAKSYPNEEKDAWDVKMLLEQFSFDI | |
| Protease | AEEASKVCLAHLFTYQDFDMGTLGLAYVGSPRANSHGGVCPKAYYSPVGKKNIYLNSGL | |
| Domain | TSTKNYGKTILTKEADLVTTHELGHNFGAEHDPDGLAECAPNEDQGGKYVMYPIAVSGD | |
| (amino acid) | HENNKMFSNCSKQSIYKTIESKAQECFQERS | |
| (G4S)3- | GGCGGCGGGGGATCTGGAGGTGGTGGCAGCGGTGGGGGCGGTAGTAATACGTGTAA | 314 |
| ADAM17 | GCTCCTTGTGGTAGCAGACCATAGGTTCTACAGATATATGGGACGAGGAGAGGAAAG | |
| Protease | CACTACGACAAACTATTTGATCGAACTTATAGATAGAGTCGACGACATCTACCGAAA | |
| Domain | CACGTCATGGGACAATGCAGGCTTCAAGGGATACGGAATACAGATAGAGCAGATACG | |
| (nucleic acid) | GATTCTGAAAAGTCCGCAGGAAGTGAAACCGGGGGAGAAACATTATAATATGGCGA | |
| | AATCATATCCAAACGAGGAGAAAGATGCGTGGGATGTAAAAATGCTCTTGGAACAGT | |
| | TTAGTTTTGACATTGCTGAGGAAGCCAGCAAAGTGTGCCTCGCTCACCTGTTTACTTA | |
| | TCAAGATTTTGATATGGGCACCCTGGGTCTTGCATATGTAGGGTCTCCCAGAGCAAAC | |
| | TCCCACGGGGGCGTATGTCCAAAAGCTTACTACAGCCCAGTAGGAAAAAAGAATATA | |
| | TACCTGAACAGCGGGCTGACTAGTACCAAAAATTACGGAAAAACTATTCTCACGAAG | |
| | GAGGCCGACTTGGTAACTACTCATGAACTGGGGCATAACTTCGGCGCCGAGCACGAC | |
| | CCGGACGGACTTGCAGAGTGCGCCCCGAACGAGGACCAAGGTGGCAAATATGTCATG | |
| | TACCCAATCGCCGTTTCAGGGGATCATGAGAACAATAAGATGTTCAGCAACTGTTCCA | |
| | AACAATCCATTTATAAAACGATAGAAAGCAAAGCCCAGGAATGTTTTCAGGAAAGAT | |
| | CA | |

Spacer/Hinge (H):

| | | |
|---|---|---|
| CD8 | TTTPAPRPPTPAPTIASQPLSLRPEACRPAAGGAVHTRGLDFACD | 21 |
| (AA 136-182) | | |
| CD8 | ACTACCACCCCAGCACCCAGACCGCCAACTCCGGCTCCTACGATAGCGTCCCAACCTC | 315 |
| (nucleic acid) | TCTCTCTTAGGCCAGAGGCATGCCGCCCGGCAGCTGGGGGTGCGGTTCATACTCGGG | |
| | GACTTGATTTCGCTTGTGAC | |
| CD8 Hinge | TTTPAPRPPTPAPTIASQPLSLRPEAARPAAGGAVHTRGLDFAAD | 288 |
| (amino acid) | | |
| CD8 Hinge | ACTACCACCCCAGCACCCAGACCGCCAACTCCGGCTCCTACGATAGCGTCCCAACCTC | 316 |
| (nucleic acid) | TCTCTCTTAGGCCAGAGGCAGCTCGCCCGGCAGCTGGGGGTGCGGTTCATACTCGGG | |
| | GACTTGATTTCGCTGCTGAC | |
| CD28 | JEVMYPPPYLDNEKSNGTIIHVKGKHLCPSPLFPGPSKP | 22 |
| (AA 114-151) | | |
| CD28 | ATCGAAGTGATGTACCCTCCACCTTACCTGGACAACGAGAAGTCCAACGGCACCATC | 317 |
| (nucleic acid) | ATCCACGTGAAGGGCAAGCACCTGTGTCCTTCTCCACTGTTCCCCGGACCTAGCAAGC | |
| | CT | |
| CD45 Hinge | TTTLSPSGSAVISTTTIATTPSKPTCDEKYANITVDYLYNKETKLFTAKLNVNENVECGNN | 289 |
| (amino acid) | TCTNNEVHNLTECKNASVSISHNSCTAPDKTLILDVPPGVEKFQLHDCTQVEKADTTICLK | |
| | WKNIETFTCDTQNITYRFQCGNMIFDNKEIKLENLEPEHEYKCDSEILYNNHKFTNASKIIK | |
| | TDFGSPGEPQIIFCRSEAAHQGVITWNPPQRSFHNFTLCYIKETEKDCLNLDKNLIKYDQN | |
| | LKPYTKYVLSLHAYIIAKVQRNGSAAMCHFTTKSAPPSQVWNMTVSMTSDNSMHVKCR | |
| | PPRDRNGPHERYHLEVEAGNTLVRNESHKNCDFRVKDLQYSTDYTFKAYFHNGDYPGEP | |
| | FILHHSTSYNSK | |
| CD45 Hinge | ACAACGACCTTATCTCCATCCGGTTCCGCAGTCATTAGCACCACGACCATAGCTACCA | 318 |
| (nucleic acid) | CGCCAAGTAAGCCAACTTGCGATGAGAAATACGCTAACATCACCGTGGATTATCTGT | |
| | ATAATAAAGAAACGAAGTTGTTCACAGCTAAGCTCAATGTAAACGAGAACGTGGAGT | |
| | GCGGGAATAATACTTGCACTAACAACGAAGTCCATAATCTCACTGAGTGCAAGAATG | |
| | CTTCTGTGTCCATTTCTCATAACTCTTGCACTGCCCCAGATAAGACACTGATACTGGAT | |
| | GTGCCACCAGGCGTGGAAAAGTTTCAGCTTCACGACTGTACCCAGGTCGAGAAAGCT | |
| | GACACGACTATCTGTCTGAAATGGAAGAACATAGAAACGTTTACATGCGATACACAG | |
| | AACATTACCTATCGATTCCAATGCGGGAACATGATTTTCGACAATAAGGAAATTAAA | |
| | CTGGAGAATCTGGAGCCTGAACATGAGTACAAATGTGACTCCGAAATCCTGTACAAT | |
| | AATCACAAGTTCACAAATGCGAGCAAAATTATCAAGACTGACTTCGGATCACCAGGC | |
| | GAACCCCAGATTATTTTCTGTCGCAGTGAGGCTGCACATCAGGGAGTCATAACATGG | |
| | AACCCGCCACAGAGAAGTTTCCATAACTTTACACTCTGTTACATAAAGGAGACTGAG | |
| | AAAGATTGCCTTAACTTAGATAAGAACCTGATCAAGTACGACCTTCAGAACTTGAAA | |
| | CCGTATACCAAGTATGTCCTTTCCCTGCACGCCTACATAATCGCCAAAGTGCAGCGAA | |
| | ATGGATCTGCTGCCATGTGCCATTTCACTACGAAATCTGCACCACCTTCCCAGGTGTG | |
| | GAACATGACAGTGTCTATGACCAGTGACAATTCAATGCACGTTAAATGTAGACCACC | |
| | GAGGGATAGAAATGGTCCACACGAGAGGTACCATTTAGAGGTAGAGGCAGGAAATA | |
| | CCTTAGTGAGAAATGAGAGCCACAAGAACTGCGACTTCAGAGTGAAAGACTTACAGT | |
| | ACTCCACTGATTATACATTTAAAGCTTATTTCCATAATGGAGATTACCCCGGCGAACC | |
| | TTTTATACTCCACCACTCTACTAGCTACAACTCCAAA | |
| G4S-CD45 | GGGGSTTTLSPSGSAVISTTTIATTPSKPTCDEKYANITVDYLYNKETKLFTAKLNVNENV | 319 |
| Hinge | ECGNNTCTNNEVHNLTECKNASVSISHNSCTAPDKTLILDVPPGVEKFQLHDCTQVEKAD | |
| (amino acid) | TTICLKWKNIETFTCDTQNITYRFQCGNMIFDNKEIKLENLEPEHEYKCDSEILYNNHKFTN | |
| | ASKIIKTDFGSPGEPQIIFCRSEAAHQGVITWNPPQRSFHNFTLCYIKETEKDCLNLDKNLIK | |
| | YDLQNLKPYTKYVLSLHAYIIAKVQRNGSAAMCHFTTKSAPPSQVWNMTVSMTSDNSM | |
| | HVKCRPPRDRNGPHERYHLEVEAGNTLVRNESHKNCDFRVKDLQYSTDYTFKAYFHNG | |
| | DYPGEPFILHHSTSYNSK | |
| G4S-CD45 | GGTGGAGGCGGTTCCACAACGACCTTATCTCCATCCGGTTCCGCAGTCATTAGCACCA | 320 |
| Hinge | CGACCATAGCTACCACGCCAAGTAAGCCAACTTGCGATGAGAAATACGCTAACATCA | |
| (nucleic acid) | CCGTGGATTATCTGTATAATAAAGAAACGAAGTTGTTCACAGCTAAGCTCAATGTAA | |
| | ACGAGAACGTGGAGTGCGGGAATAATACTTGCACTAACAACGAAGTCCATAATCTCA | |
| | CTGAGTGCAAGAATGCTTCTGTGTCCATTTCTCATAACTCTTGCACTGCCCCAGATAA | |
| | GACACTGATACTGGATGTGCCACCAGGCGTGGAAAAGTTTCAGCTTCACGACTGTAC | |
| | CCAGGTCGAGAAAGCTGACACGACTATCTGTCTGAAATGGAAGAACATAGAAACGTT | |
| | TACATGCGATACACAGAACATTACCTATCGATTCCAATGCGGGAACATGATTTTCGAC | |
| | AATAAGGAAATTAAACTGGAGAATCTGGAGCCTGAACATGAGTACAAATGTGACTCC | |
| | GAAATCCTGTACAATAATCACAAGTTCACAAATGCGAGCAAAATTATCAAGACTGAC | |
| | TTCGGATCACCAGGCGAACCCCAGATTATTTTCTGTCGCAGTGAGGCTGCACATCAGG | |
| | GAGTCATAACATGGAACCCGCCACAGAGAAGTTTCCATAACTTTACACTCTGTTACAT | |
| | AAAGGAGACTGAGAAAGATTGCCTTAACTTAGATAAGAACCTGATCAAGTACGACCT | |
| | TCAGAACTTGAAACCGTATACCAAGTATGTCCTTTCCCTGCACGCCTACATAATCGCC | |
| | AAAGTGCAGCGAAATGGATCTGCTGCCATGTGCCATTTCACTACGAAATCTGCACCAC | |
| | CTTCCCAGGTGTGGAACATGACAGTGTCTATGACCAGTGACAATTCAATGCACGTTAA | |
| | ATGTAGACCACCGAGGGATAGAAATGGTCCACACGAGAGGTACCATTTAGAGGTAGA | |
| | GGCAGGAAATACCTTAGTGAGAAATGAGAGCCACAAGAACTGCGACTTCAGAGTGA | |
| | AAGACTTACAGTACTCCACTGATTATACATTTAAAGCTTATTTCCATAATGGAGATTA | |
| | CCCCGGCGAACCTTTTATACTCCACCACTCTACTAGCTACAACTCCAAA | |
| CXC3R GPCR | VLEVSDHQVLNDAEVAALLENFSSSYDYGENESDSCCTSPPCPQDFSLNFDRAFLPALYSL | 321 |
| Hinge | LFLLGLLGNGAVAAVLLSRRTALSSTDTFLLHLAVADTLLVLTLPLWAVDAAVQWVFGS | |
| (amino acid) | GLCKVAGALFNINFYAGALLLACISFDRYLNIVH | |
| CXC3R GPCR | GTCCTTGAGGTGTCCGACCACCAGGTCCTGAACGATGCTGAGGTGGCAGCGCTGTTG | 322 |
| Hinge | GAGAACTTCAGTTCATCCTACGATTACGGAGAGAACGAGTCTGATAGTTGTTGTACCA | |
| (nucleic acid) | GTCCCCCCTGCCCCCAGGATTTTTCCCTTAATTTCGACAGGGCTTTCCTCCCCGCACTG | |
| | TATTCCCTTCTGTTTCTTTTAGGGCTGTTAGGCAATGGCGCCGTGGCGGCTGTCCTTCT | |
| | GAGTCGGCGCACAGCCCTCTCCAGTACGGATACCTTTCTCCTGCACCTGGCGGTGGCC | |
| | GACACGCTGTTGGTCCTGACTCTTCCACTCTGGGCCGTTGATGCCGCAGTCCAATGGG | |
| | TTTTTGGCTCCGGTCTGTGCAAGGTAGCGGGAGCTCTGTTCAATATCAACTTCTACGC | |
| | TGGAGCTCTGCTGCTGGCCTGCATCAGCTTCGATAGATATCTGAACATCGTCCAT | |

Transmembrane Domain (TMD):

| | | |
|---|---|---|
| CD8 TMD | IYIWAPLAGTCGVLLLSLVIT | 23 |
| (amino acid) | | |
| CD8 TMD | ATATACATTTGGGCTCCATTGGCGGGCACGTGCGGAGTTCTGCTTCTCAGTTTGGTTA | 374 |
| (nucleic acid) | TTACC | |
| PD1 TMD | VGVVGGLLGSLVLLVWVLAVI | 290 |
| (amino acid) | | |
| PD1 TMD | GTGGGAGTTGTTGGGGGACTTCTTGGATCTCTTGTCCTTCTTGTATGGGTGTTGGCAGT | 375 |
| (nucleic acid) | CATA | |
| CD28 TMD | FWVLVVVGGVLACYSLLVTVAFIIFWV | 24 |
| (amino acid) | | |
| CD28 TMD | TTTTGGGTGCTGGTGGTGGTTGGTGGAGTCCTGGCTTGCTATTCCTTGCTAGTAACAGT | 376 |
| (nucleic acid) | GGCCTTCATCATCTTTTGGGTC | |
| SynNotch TMD | ILDYSFTGGAGRDIPPPQIEEACELPECQVDAGNKVCNLQCNNHACGWDGGDCSLNFNDP | 291 |
| (amino acid) | WKNCTQSLQCWKYFSDGHCDSQCNSAGCLFDGFDCQLTEGQCNPLYDQYCKDHFSDGH | |
| | CDQGCNSAECEWDGLDCAEHVPERLAAGTLVLVVLLPPDQLRNNSFHFLRELSHVLHTN | |
| | VVFKRDAQGQQMIFPYYGHEEELRKHPIKRSTVGWATSSLLPGTSGGRQRRELDPMDIRG | |
| | SIVYLEIDNRQCVQSSSQCFQSATDVAAFLGALASLGSLNIPYKIEAVKSEPVEPPLPSQLH | |
| | LMYVAAAAFVLLFFVGCGVLLSRKRRR | |
| SynNotch TMD | ATTTTGGATTATAGTTTCACCGGAGGCGCCGGACGCGACATACCTCCCCCGCAAATCG | 377 |
| (nucleic acid) | AGGAAGCGTGCGAACTCCCAGAGTGTCAAGTAGACGCCGGCAACAAGGTGTGCAACC | |
| | TGCAATGCAACAACCATGCCTGCGGATGGGATGGAGGGGACTGTAGTTTGAATTTCA | |
| | ACGACCCTTGGAAGAACTGCACGCAGTCTCTTCAATGCTGGAAATATTTCAGCGATGG | |
| | ACATTGTGACAGCCAGTGTAACAGCGCCGGATGCTTGTTTGATGGCTTTGATTGTCAA | |
| | TTGACCGAGGGTCAGTGCAACCCGCTCTATGACCAATATTGTAAGGATCACTTTTCCG | |
| | ACGGGCACTGCGATCAGGGCTGCAATAGCGCGGAATGTGAATGGGACGGATTGGATT | |
| | GTGCAGAACATGTGCCCGAACGCCTCGCGGCAGGAACTCTGGTCCTCGTAGTCTTGTT | |
| | GCCGCCCGACCAATTGCGGAACAACAGTTTCCACTTTCTGCGGGAATTGTCACATGTG | |
| | CTGCATACAAATGTCGTGTTTAAGCGCGATGCTCAGGGTCAACAAATGATATTCCCGT | |
| | ACTATGGTCACGAAGAGGAGCTTAGGAAGCACCCGATTAAGCGGTCTACGGTTGGAT | |
| | GGGCTACTTCCTCTCTTCTCCCGGGGACGAGCGGGGGCAGACAAAGGCGGGAGCTCG | |
| | ACCCAATGGACATTAGAGGATCAATAGTGTACCTGGAGATTGACAATCGGCAATGCG | |
| | TCCAATCTAGCAGTCAATGCTTTCAGTCTGCAACCGATGTCGCAGCCTTTCTGGGCGC | |
| | CCTGGCCAGCCTCGGGTCCCTTAATATCCCGTATAAGATTGAGGCGGTTAAATCTGAA | |
| | CCTGTCGAACCTCCCCTCCCCTCTCAATTGCACCTCATGTACGTTGCTGCTGCTGCCTT | |
| | TGTTCTGCTCTTCTTCGTAGGATGCGGGGTTCTTCTCTCCAGAAAACGACGGCGC | |
| CXC3R GPCR | PARVTLTCLAVWGLCLLFALPDFIFLSAHHDERLNATHCQYNFPQVGRTALRVLQLVAGF | 323 |
| TMD | LLPLLVMAYCYAHILAVLL | |
| (amino acid) | | |
| CXC3R GPCR | CCAGCCAGAGTAACACTGACATGTCTCGCTGTCTGGGGTCTGTGCCTGCTTTTCGCCC | 324 |
| TMD | TGCCTGACTTCATTTTCCTGTCTGCTCACCACGACGAGCGTCTGAATGCCACCCACTG | |
| (nucleic acid) | CCAGTATAATTTCCCTCAAGTAGGTCGGACCGCCCTTCGAGTTTTGCAGCTGGTGGCC | |
| | GGTTTCTTATTGCCCTTACTGGTGATGGCTTACTGCTACGCACACATCTTGGCTGTGCT | |
| | TCTCGTT | |
| CXC3R GPCR | QRRLRAMRLVVVVVVAFALCWTPYHLVVLVDILMDLGALARNCGRESRVDVAKSVTSG | 325 |
| TMD | LGYMHCCLNPLLYAFV | |
| (amino acid) | | |
| CXC3R GPCR | CAGAGGAGGCTGAGGGCCATGAGACTCGTTGTCGTCGTGGTTGTTGCTTTCGCCTTAT | 326 |
| TMD | GCTGGACCCCATACCATTTAGTTGTGCTGGTGGATATCCTGATGGACCTGGGCGCCCT | |
| (nucleic acid) | CGCACGGAACTGTGGCAGGGAATCAAGAGTGGACGTCGCTAAATCCGTGACCTCCGG | |
| | ACTGGGTTATATGCACTGTTGCCTCAACCCTCTGCTCTACGCCTTTGTG | |

Linkers:

| | | |
|---|---|---|
| (G₄S)₂ | GGGGSGGGGS | 103 |
| Linker 35 | | |
| (G₄S)₃ | GGGGSGGGGSGGGGS | 25 |

Signal Peptide (SP):

| | | |
|---|---|---|
| CD8-SP | MALPVTALLLPLALLLHAARP | 292 |
| (amino acid) | | |
| CD8-SP | ATGGCCCTTCCCGTGACTGCGCTCCTCCTGCCTCTTGCCCTCCTTCTTCATGCTGCTCG | 327 |
| (nucleic acid) | CCCG | |
| IgK Signal | MARSPAQLLGLLLLWLSGARC | 97 |
| Peptide Variant | | |
| (amino acid) | | |
| IgK Signal | ATGGCCAGATCTCCTGCTCAACTGCTGGGACTGCTGCTGCTGTGGCTTAGCGGAGCCA | 98 |
| Peptide Variant | GATGC | |
| (nucleic acid) | | |
| MARS SP | ATGGCCAGATCCCCGGCACAACTGCTCGGACTCCTCCTGTTGTGGTTGAGCGGGGCCC | 378 |
| (nucleic acid) | GCTGT | |

iCAR Sequences Without Antigen Binding Domains:

| | |
|---|---|
| CD8 (SP)- | MALPVTALLLPLALLLHAARP (SEQ ID NO: 292) |
| iCAR Antigen | [[--INSERT AA SEQUENCE OF iCAR ANTIGEN BINDING DOMAIN SELECTED FROM |
| Binding | TABLE 5--]] |
| Domain-CD8 | TTTPAPRPPTPAPTIASQPLSLRPEACRPAAGGAVHTRGLDFACDIYIWAPLAGTCGVLLLSLVITCSRAA |
| (H)-CD8 | RGTIGARRTGQPLKEDPSAVPVFSVDYGELDFQWREKTPEPPVPCVPEQTEYATIVFPSGMGTSSPARRG |
| (TMD)-PD1 | SADGPRSAQPLRPEDGHCSWPL (SEQ ID NO: 242) |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-PD1 | GGCCAGAGGCATGCCGCCCGGCAGCTGGGGGTGCGGTTCATACTCGGGGACTTGATTTCGCTTGTG |
| (nucleic acid) | ACATATACATTTGGGCTCCATTGGCGGGCACGTGCGGAGTTCTGCTTCTCAGTTTGGTTATTACCTG |
| | TAGTCGCGCTGCAAGAGGTACCATTGGTGCGAGGAGGACCGGACAACCTTTGAAGGAGGACCCAA |
| | GTGCGGTACCTGTTTTTTCCGTCGACTACGGCGAGCTGGATTTCCAGTGGCGAGAAAAGACACCAG |
| | AACCACCGGTTCCTTGTGTACCTGAGCAGACGGAGTATGCGACAATCGTCTTCCCGAGCGGAATGG |
| | GCACGAGTTCCCCCGCCAGAAGGGGGTCTGCCGACGGACCTAGGAGCGCACAACCACTTAGACCCG |
| | AAGACGGCCATTGTTCCTGGCCACTG (SEQ ID NO: 328) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEAARPAAGGAVHTRGLDFAADVGVVGGLLGSLVLLVWVLAVICSRA |
| (H)-PD1 | ARGTIGARRTGQPLKEDPSAVPVFSVDYGELDFQWREKTPEPPVPCVPEQTEYATIVFPSGMGTSSPARR |
| (TMD)-PD1 | GSADGPRSAQPLRPEDGHCSWPL (SEQ ID NO: 243) |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-PD1 | ACTACCACCCCAGCACCCAGACCGCCAACTCCGGCTCCTACGATAGCGTCCCAACCTCTCTCTCTTA |
| (TMD)-PD1 | GGCCAGAGGCAGCTCGCCCGGCAGCTGGGGGTGCGGTTCATACTCGGGGACTTGATTTCGCTGCTG |
| (nucleic acid) | ACGTGGGAGTTGTTGGGGGACTTCTTGGATCTCTTGTCCTTCTTGTATGGGTGTTGGCAGTCATATGT |
| | AGTCGCGCTGCAAGAGGTACCATTGGTGCGAGGAGGACCGGACAACCTTTGAAGGAGGACCCAAG |
| | TGCGGTACCTGTTTTTTCCGTCGACTACGGCGAGCTGGATTTCCAGTGGCGAGAAAAGACACCAGA |
| | ACCACCGGTTCCTTGTGTACCTGAGCAGACGGAGTATGCGACAATCGTCTTCCCGAGCGGAATGGG |
| | CACGAGTTCCCCCGCCAGAAGGGGGTCTGCCGACGGACCTAGGAGCGCACAACCACTTAGACCCGA |
| | AGACGGCCATTGTTCCTGGCCACTG (SEQ ID NO: 329) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD28 | IEVMYPPPYLDNEKSNGTIIHVKGKHLCPSPLFPGPSKPFWVLVVVGGVLACYSLLVTVAFIIFWVCSRA |
| (H)-CD28 | ARGTIGARRTGQPLKEDPSAVPVFSVDYGELDFQWREKTPEPPVPCVPEQTEYATIVFPSGMGTSSPARR |
| (TMD)-PD1 | GSADGPRSAQPLRPEDGHCSWPL (SEQ ID NO: 244) |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD28 | FROM TABLE 6--]] |
| (H)-CD28 | ATCGAAGTGATGTACCCTCCACCTTACCTGGACAACGAGAAGTCCAACGGCACCATCATCCACGTG |
| (TMD)-PD1 | AAGGGCAAGCACCTGTGTCCTTCTCCACTGTTCCCCGGACCTAGCAAGCCTTTTTGGGTGCTGGTGG |
| (nucleic acid) | TGGTTGGTGGAGTCCTGGCTTGCTATTCCTTGCTAGTAACAGTGGCCTTCATCATCTTTTGGGTCTGT |
| | AGTCGCGCTGCAAGAGGTACCATTGGTGCGAGGAGGACCGGACAACCTTTGAAGGAGGACCCAAG |
| | TGCGGTACCTGTTTTTTCCGTCGACTACGGCGAGCTGGATTTCCAGTGGCGAGAAAAGACACCAGA |
| | ACCACCGGTTCCTTGTGTACCTGAGCAGACGGAGTATGCGACAATCGTCTTCCCGAGCGGAATGGG |
| | CACGAGTTCCCCCGCCAGAAGGGGGTCTGCCGACGGACCTAGGAGCGCACAACCACTTAGACCCGA |
| | AGACGGCCATTGTTCCTGGCCACTG (SEQ ID NO: 330) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDFWVLVVVGGVLACYSLLVTVAFIIF |
| (H)-CD28 | WVCSRAARGTIGARRTGQPLKEDPSAVPVFSVDYGELDFQWREKTPEPPVPCVPEQTEYATIVFPSGMG |
| (TMD)-PD1 | TSSPARRGSADGPRSAQPLRPEDGHCSWPL (SEQ ID NO: 245) |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD28 | ACTACCACCCCAGCACCCAGACCGCCAACTCCGGCTCCTACGATAGCGTCCCAACCTCTCTCTCTTA |
| (TMD)-PD1 | GGCCAGAGGCATGCCGCCCGGCAGCTGGGGGTGCGGTTCATACTCGGGGACTTGATTTCGCTTGTG |
| (nucleic acid) | ACTTTTGGGTGCTGGTGGTGGTTGGTGGAGTCCTGGCTTGCTATTCCTTGCTAGTAACAGTGGCCTT |
| | CATCATCTTTTGGGTCTGTAGTCGCGCTGCAAGAGGTACCATTGGTGCGAGGAGGACCGGACAACC |
| | TTTGAAGGAGGACCCAAGTGCGGTACCTGTTTTTTCCGTCGACTACGGCGAGCTGGATTTCCAGTGG |
| | CGAGAAAAGACACCAGAACCACCGGTTCCTTGTGTACCTGAGCAGACGGAGTATGCGACAATCGTC |
| | TTCCCGAGCGGAATGGGCACGAGTTCCCCCGCCAGAAGGGGGTCTGCCGACGGACCTAGGAGCGC |
| | ACAACCACTTAGACCCGAAGACGGCCATTGTTCCTGGCCACTG (SEQ ID NO: 331) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITLRHRR |
| (H)-CD8 | QGKHWTSTQRKADFQHPAGAVGPEPTDRGLQWRSSPAADAQEENLYAAVKHTQPEDGVEMDTRSPH |
| (TMD)-LIRB1 | DEDPQAVTYAEVKHSRPRREMASPPSPLSGEFLDTKDRQAEEDRQMDTEAAASEAPQDVTYAQLHSLT |
| (amino acid) | LRREATEPPPSQEGPSPAVPSIYATLAIH (SEQ ID NO: 246) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-LIRB1 | GGCCAGAGGCATGCCGCCCGGCAGCTGGGGGTGCGGTTCATACTCGGGGACTTGATTTCGCTTGTG |
| (nucleic acid) | ACATATACATTTGGGCTCCATTGGCGGGCACGTGCGGAGTTCTGCTTCTCAGTTTGGTTATTACCCT |
| | CCGACATAGACGCCAAGGCAAACACTGGACGTCTACTCAACGAAAAGCTGATTTTCAGCATCCCGC |
| | CGGCGCCGTAGGTCCTGAACCAACGGATCGGGGTTTGCAATGGCGAAGTAGTCCTGCTGCTGATGC |
| | ACAGGAGGAGAACCTTTATGCGGCTGTAAAACACACTCAACCCGAAGATGGGGTCGAAATGGACA |
| | CGCGAAGCCCCCATGACGAAGATCCCCAGGCGGTCACATATGCAGAAGTCAAACACAGCCGCCCA |
| | AGGCGGGAAATGGCGAGTCCCCCAAGCCCACTCTCAGGAGAGTTCCTTGATACCAAGGACCGCCAG |
| | GCCGAGGAGGACAGGCAGATGGATACTGAGGCTGCCGCATCAGAGGCACCACAAGATGTTACCTA |
| | CGCGCAACTGCACTCATTGACGCTTCGAAGAGAGGCCACCGAACCCCCACCAAGTCAGGAAGGCCC |
| | GAGCCCTGCGGTCCCATCTATATACGCAACCCTGGCCATTCAT (SEQ ID NO: 332) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITCSRAA |
| (H)-CD8 | RGTIGARRTGQPLKEDPSAVPVFSVDYGELDFQWREKTPEPPVPCVPEQTEYATIVFPSGMGTSSPARRG |
| (TMD)-PD1- | SADGPRSAQPLRPEDGHCSWPLLTRKKKALRIHSVEGDLRRKSAGQEEWSPSAPSPPGSCVQAEAAPAG |
| TIGIT | LCGEQRGEDCAELHDYFNVLSYRSLGNCSFFTETG (SEQ ID NO: 247) |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-PD1- | GGCCAGAGGCATGCCGCCCGGCAGCTGGGGGTGCGGTTCATACTCGGGGACTTGATTTCGCTTGTG |
| TIGIT | ACATATACATTTGGGCTCCATTGGCGGGCACGTGCGGAGTTCTGCTTCTCAGTTTGGTTATTACCTG |
| (nucleic acid) | TAGTCGCGCTGCAAGAGGTACCATTGGTGCGAGGAGGACCGGACAACCTTTGAAGGAGGACCCAA |
| | GTGCGGTACCTGTTTTTTCCGTCGACTACGGCGAGCTGGATTTCCAGTGGCGAGAAAAGACACCAG |
| | AACCACCGGTTCCTTGTGTACCTGAGCAGACGGAGTATGCGACAATCGTCTTCCCGAGCGGAATGG |
| | GCACGAGTTCCCCCGCCAGAAGGGGGTCTGCCGACGGACCTAGGAGCGCACAACCACTTAGACCCG |
| | AAGACGGCCATTGTTCCTGGCCACTGCTCACCCGAAAGAAGAAGGCTTTGAGGATTCATTCCGTAG |
| | AAGGCGACCTGCGACGCAAGTCAGCTGGTCAAGAGGAATGGTCACCATCCGCACCGTCTCCGCCTG |
| | GGTCATGTGTTCAAGCTGAAGCGGCTCCTGCCGGGCTTTGTGGTGAACAACGGGGCGAAGATTGCG |
| | CCGAGTTGCATGATTATTTCAACGTTCTTAGTTACCGCTCTCTCGGAAATTGTAGTTTTTTTACCGAG |
| | ACCGGA (SEQ ID NO: 333) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITCSRAA |
| (H)-CD8 | RGTIGARRTGQPLKEDPSAVPVFSVDYGELDFQWREKTPEPPVPCVPEQTEYATIVFPSGMGTSSPARRG |
| (TMD)-PD1- | SADGPRSAQPLRPEDGHCSWPLAVSLSKMLKKRSPLTTGVYVKMPPTEPECEKQFQPYFIPIN (SEQ ID |
| CTLA4 | NO: 248) |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-PD1- | GGCCAGAGGCATGCCGCCCGGCAGCTGGGGGTGCGGTTCATACTCGGGGACTTGATTTCGCTTGTG |
| CTLA4 | ACATATACATTTGGGCTCCATTGGCGGGCACGTGCGGAGTTCTGCTTCTCAGTTTGGTTATTACCTG |
| (nucleic acid) | TAGTCGCGCTGCAAGAGGTACCATTGGTGCGAGGAGGACCGGACAACCTTTGAAGGAGGACCCAA |
| | GTGCGGTACCTGTTTTTTCCGTCGACTACGGCGAGCTGGATTTCCAGTGGCGAGAAAAGACACCAG |
| | AACCACCGGTTCCTTGTGTACCTGAGCAGACGGAGTATGCGACAATCGTCTTCCCGAGCGGAATGG |
| | GCACGAGTTCCCCCGCCAGAAGGGGGTCTGCCGACGGACCTAGGAGCGCACAACCACTTAGACCCG |
| | AAGACGGCCATTGTTCCTGGCCACTGGCAGTCTCACTCTCCAAAATGCTGAAAAAGCGCTCTCCCCT |
| | TACCACCGGCGTCTATGTCAAAATGCCACCTACTGAACCTGAATGCGAGAAGCAGTTCCAACCTTA |
| | TTTTATCCCCATTAAT (SEQ ID NO: 334) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITCSRAA |
| (H)-CD8 | RGTIGARRTGQPLKEDPSAVPVFSVDYGELDFQWREKTPEPPVPCVPEQTEYATIVFPSGMGTSSPARRG |
| (TMD)-PD1- | SADGPRSAQPLRPEDGHCSWPLAVSLSKMLKKRSPLTTGVYVKMPPTEPECEKQFQPYFIPINYSSV |
| CTLA4- | (SEQ ID NO: 249) |
| CSK*(YSSV) | |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-PD1- | GGCCAGAGGCATGCCGCCCGGCAGCTGGGGGTGCGGTTCATACTCGGGGACTTGATTTCGCTTGTG |
| CTLA4- | ACATATACATTTGGGCTCCATTGGCGGGCACGTGCGGAGTTCTGCTTCTCAGTTTGGTTATTACCTG |
| CSK*(YSSV) | TAGTCGCGCTGCAAGAGGTACCATTGGTGCGAGGAGGACCGGACAACCTTTGAAGGAGGACCCAA |
| (nucleic acid) | GTGCGGTACCTGTTTTTTCCGTCGACTACGGCGAGCTGGATTTCCAGTGGCGAGAAAAGACACCAG |
| | AACCACCGGTTCCTTGTGTACCTGAGCAGACGGAGTATGCGACAATCGTCTTCCCGAGCGGAATGG |
| | GCACGAGTTCCCCCGCCAGAAGGGGGTCTGCCGACGGACCTAGGAGCGCACAACCACTTAGACCCG |
| | AAGACGGCCATTGTTCCTGGCCACTGGCAGTCTCACTCTCCAAAATGCTGAAAAAGCGCTCTCCCCT |
| | TACCACCGGCGTCTATGTCAAAATGCCACCTACTGAACCTGAATGCGAGAAGCAGTTCCAACCTTA |
| | TTTTATCCCCATTAATTACTCATCTGTT (SEQ ID NO: 335) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITCSRAA |
| (H)-CD8 | RGTIGARRTGQPLKEDPSAVPVFSVDYGELDFQWREKTPEPPVPCVPEQTEYATIVFPSGMGTSSPARRG |
| (TMD)-PD1- | SADGPRSAQPLRPEDGHCSWPLYSSV (SEQ ID NO: 250) |
| CSK*(YSSV) | |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-PD1- | GGCCAGAGGCATGCCGCCCGGCAGCTGGGGGTGCGGTTCATACTCGGGGACTTGATTTCGCTTGTG |
| CSK*(YSSV) | ACATATACATTTGGGCTCCATTGGCGGGCACGTGCGGAGTTCTGCTTCTCAGTTTGGTTATTACCTG |
| (nucleic acid) | TAGTCGCGCTGCAAGAGGTACCATTGGTGCGAGGAGGACCGGACAACCTTTGAAGGAGGACCCAA |
| | GTGCGGTACCTGTTTTTTCCGTCGACTACGGCGAGCTGGATTTCCAGTGGCGAGAAAAGACACCAG |
| | AACCACCGGTTCCTTGTGTACCTGAGCAGACGGAGTATGCGACAATCGTCTTCCCGAGCGGAATGG |
| | GCACGAGTTCCCCCGCCAGAAGGGGGTCTGCCGACGGACCTAGGAGCGCACAACCACTTAGACCCG |
| | AAGACGGCCATTGTTCCTGGCCACTGTACTCATCTGTT (SEQ ID NO: 336) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITHRWCS |
| (H)-CD8 | NKKNAAVMDQESAGNRTANSEDSDEQDPQEVTYTQLNHCVFTQRKITRPSQRPKTPPTDIIVYTELPNA |
| (TMD)- | ESRSKVVSCP (SEQ ID NO: 251) |
| KIR2DL 1 | |
| (amino acid) | |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)- | GGCCAGAGGCATGCCGCCCGGCAGCTGGGGGTGCGGTTCATACTCGGGGACTTGATTTCGCTTGTG |
| KIR2DL1 | ACATATACATTTGGGCTCCATTGGCGGGCACGTGCGGAGTTCTGCTTCTCAGTTTGGTTATTACCCA |
| (nucleic acid) | CCGATGGTGTTCTAACAAAAAGAACGCAGCCGTTATGGACCAGGAAAGCGCGGGAAACCGGACGG |
| | CGAACAGCGAAGACTCCGATGAGCAGGATCCGCAGGAGGTAACTTACACTCAGCTTAATCACTGTG |
| | TATTCACTCAAAGGAAGATCACCCGGCCTTCTCAAAGACCGAAAACTCCGCCTACGGACATTATTG |
| | TTTATACGGAGCTTCCAAACGCTGAGTCTCGGAGTAAGGTGGTGTCTTGCCCA (SEQ ID NO: 337) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITKRKEV |
| (H)-CD8 | QKTCRKHRKENQGSHESPTLNPETVAINLSDVDLSKYITTIAGVMTLSQVKGFVRKNGVNEAKIDEIKND |
| (TMD)-DR1 | NVQDTAEQKVQLLRNWHQLHGKKEAYDTLIKDLKKANLCTLAEKIQTIILKDITSDSENSNFRNEIQSLV |
| (amino acid) | (SEQ ID NO: 252) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-DR1 | GGCCAGAGGCATGCCGCCCGGCAGCTGGGGGTGCGGTTCATACTCGGGGACTTGATTTCGCTTGTG |
| (nucleic acid) | ACATATACATTTGGGCTCCATTGGCGGGCACGTGCGGAGTTCTGCTTCTCAGTTTGGTTATTACCAA |
| | GCGAAAAGAGGTGCAAAAGACGTGTAGAAAGCACCGGAAGGAGAACCAGGGGTCACACGAGAGC |
| | CCGACACTGAACCCAGAGACGGTTGCAATAAATCTTTCTGATGTGGATTTGAGCAAGTATATCACA |
| | ACAATCGCCGGAGTCATGACGCTGAGCCAGGTTAAGGGCTTTGTACGCAAGAATGGCGTAAACGAG |
| | GCAAAAATTGACGAGATTAAAAATGACAACGTGCAGGACACCGCTGAGCAGAAGGTACAACTTTT |
| | GAGGAACTGGCATCAACTTCATGGAAAGAAGGAGGCGTATGACACTCTTATTAAGGACTTGAAGAA |
| | GGCCAATCTCTGTACACTTGCAGAGAAAATACAAACGATTATCTTGAAGGATATAACTAGTGACAG |
| | TGAGAACTCTAATTTTAGAAATGAAATCCAGTCTCTGGTC (SEQ ID NO: 338) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD28 | IEVMYPPPYLDNEKSNGTIIHVKGKHLCPSPLFPGPSKPFWVLVVVGGVLACYSLLVTVAFIIFWVKRKE |
| (H)-CD28 | VQKTCRKHRKENQGSHESPTLNPETVAINLSDVDLSKYITTIAGVMTLSQVKGFVRKNGVNEAKIDEIKN |
| (TMD)-DR1 | DNVQDTAEQKVQLLRNWHQLHGKKEAYDTLIKDLKKANLCTLAEKIQTIILKDITSDSENSNFRNEIQSL |
| (amino acid) | V (SEQ ID NO: 253) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD28 | FROM TABLE 6--]] |
| (H)-CD28 | ATCGAAGTGATGTACCCTCCACCTTACCTGGACAACGAGAAGTCCAACGGCACCATCATCCACGTG |
| (TMD)-DR1 | AAGGGCAAGCACCTGTGTCCTTCTCCACTGTTCCCCGGACCTAGCAAGCCTTTTTGGGTGCTGGTGG |
| (nucleic acid) | TGGTTGGTGGAGTCCTGGCTTGCTATTCCTTGCTAGTAACAGTGGCCTTCATCATCTTTTGGGTCAAG |
| | CGAAAAGAGGTGCAAAAGACGTGTAGAAAGCACCGGAAGGAGAACCAGGGGTCACACGAGAGCC |
| | CGACACTGAACCCAGAGACGGTTGCAATAAATCTTTCTGATGTGGATTTGAGCAAGTATATCACAA |
| | CAATCGCCGGAGTCATGACGCTGAGCCAGGTTAAGGGCTTTGTACGCAAGAATGGCGTAAACGAGG |
| | CAAAAATTGACGAGATTAAAAATGACAACGTGCAGGACACCGCTGAGCAGAAGGTACAACTTTTG |
| | AGGAACTGGCATCAACTTCATGGAAAGAAGGAGGCGTATGACACTCTTATTAAGGACTTGAAGAAG |
| | GCCAATCTCTGTACACTTGCAGAGAAAATACAAACGATTATCTTGAAGGATATAACTAGTGACAGT |
| | GAGAACTCTAATTTTAGAAATGAAATCCAGTCTCTGGTC (SEQ ID NO: 339) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDILDYSFTGGAGRDIPPPQIEEACELPE |
| (H)-SynNotch | CQVDAGNKVCNLQCNNHACGWDGGDCSLNFNDPWKNCTQSLQCWKYFSDGHCDSQCNSAGCLFDGF |
| (TMD)- | DCQLTEGQCNPLYDQYCKDHFSDGHCDQGCNSAECEWDGLDCAEHVPERLAAGTLVLVVLLPPDQLR |
| Casp8wt | NNSFHFLRELSHVLHTNVVFKRDAQGQQMIFPYYGHEEELRKHPIKRSTVGWATSSLLPGTSGGRQRRE |
| (amino acid) | LDPMDIRGSIVYLEIDNRQCVQSSSQCFQSATDVAAFLGALASLGSLNIPYKIEAVKSEPVEPPLPSQLHL |
| | MYVAAAAFVLLFFVGCGVLLSRKRRRMDFSRNLYDIGEQLDSEDLASLKFLSLDYIPQRKQEPIKDALM |
| | LFQRLQEKRMLEESNLSFLKELLFRINRLDLLITYLNTRKEEMERELQTPGRAQISAYRVMLYQISEEVSR |
| | SELRSFKFLLQEEISKCKLDDDMNLLDIFIEMEKRVILGEGKLDILKRVCAQINKSLLKIINDYEEFSKERSS |
| | SLEGSPDEFSNGEELCGVMTISDSPREQDSESQTLDKVYQMKSKPRGYCLIINNHNFAKAREKVPKLHSI |
| | RDRNGTHLDAGALTTTFEELHFEIKPHDDCTVEQIYEILKIYQLMDHSNMDCFICCILSHGDKGIIYGTDG |
| | QEAPIYELTSQFTGLKCPSLAGKPKVFFIQACQGDNYQKGIPVETDSEEQPYLEMDLSSPQTRYIPDEADF |
| | LLGMATVNNCVSYRNPAEGTWYIQSLCQSLRERCPRGDDILTILTEVNYEVSNKDDKKNMGKQMPQPT |
| | FTLRKKLVFPSD (SEQ ID NO: 254) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-SynNotch | ACTACCACCCCAGCACCCAGACCGCCAACTCCGGCTCCTACGATAGCGTCCCAACCTCTCTCTCTTA |
| (TMD)- | GGCCAGAGGCATGCCGCCCGGCAGCTGGGGGTGCGGTTCATACTCGGGGACTTGATTTCGCTTGTG |
| Casp8wt | ACATTTTGGATTATAGTTTCACCGGAGGCGCCGGACGCGACATACCTCCCCCGCAAATCGAGGAAG |
| (nucleic acid) | CGTGCGAACTCCCAGAGTGTCAAGTAGACGCCGGCAACAAGGTGTGCAACCTGCAATGCAACAACC |
| | ATGCCTGCGGATGGGATGGAGGGGACTGTAGTTTGAATTTCAACGACCCTTGGAAGAACTGCACGC |
| | AGTCTCTTCAATGCTGGAAATATTTCAGCGATGGACATTGTGACAGCCAGTGTAACAGCGCCGGAT |
| | GCTTGTTTGATGGCTTTGATTGTCAATTGACCGAGGGTCAGTGCAACCCGCTCTATGACCAATATTG |
| | TAAGGATCACTTTTCCGACGGGCACTGCGATCAGGGCTGCAATAGCGCGGAATGTGAATGGGACGG |
| | ATTGGATTGTGCAGAACATGTGCCCGAACGCCTCGCGGCAGGAACTCTGGTCCTCGTAGTCTTGTTG |
| | CCGCCCGACCAATTGCGGAACAACAGTTTCCACTTTCTGCGGGAATTGTCACATGTGCTGCATACAA |
| | ATGTCGTGTTTAAGCGCGATGCTCAGGGTCAACAAATGATATTCCCGTACTATGGTCACGAAGAGG |
| | AGCTTAGGAAGCACCCGATTAAGCGGTCTACGGTTGGATGGGCTACTTCCTCTCTTCTCCCGGGGAC |
| | GAGCGGGGGCAGACAAAGGCGGGAGCTCGACCCAATGGACATTAGAGGATCAATAGTGTACCTGG |
| | AGATTGACAATCGGCAATGCGTCCAATCTAGCAGTCAATGCTTTCAGTCTGCAACCGATGTCGCAG |
| | CCTTTCTGGGCGCCCTGGCCAGCCTCGGGTCCCTTAATATCCCGTATAAGATTGAGGCGGTTAAATC |
| | TGAACCTGTCGAACCTCCCCTCCCCTCTCAATTGCACCTCATGTACGTTGCTGCTGCTGCCTTTGTTC |
| | TGCTCTTCTTCGTAGGATGCGGGGTTCTTCTCTCCAGAAAACGACGGCGCATGGACTTCAGCAGAAA |
| | TTTGTATGATATCGGAGAACAACTCGATTCCGAGGATCTTGCGTCACTTAAGTTCCTCAGCCTGGAC |
| | TACATACCCCAGCGCAAGCAAGAACCTATAAAGGACGCGCTCATGTTGTTCCAACGCCTCCAAGAG |
| | AAACGCATGCTCGAAGAGTCCAATTTGAGTTTTCTCAAGGAACTGCTGTTTCGGATAAACCGACTG |
| | GATCTTCTTATTACTTATCTTAATACACGCAAGGAGGAAATGGAACGGGAGCTGCAGACTCCTGGC |
| | CGGGCACAGATTTCTGCCTATAGGGTCATGCTGTATCAGATATCCGAAGAAGTAAGTCGATCTGAA |
| | CTTCGCTCATTTAAATTCCTGTTGCAAGAGGAAATTTCTAAGTGCAAGCTGGATGACGACATGAACC |
| | TCCTGGACATATTCATAGAAATGGAAAAGCGCGTCATTTTGGGCGAAGGAAAGCTGGATATCTTGA |
| | AACGCGTCTGCGCTCAAATAAATAAATCCTTGTTGAAGATTATTAATGACTACGAGGAGTTTTCTAA |
| | AGAACGCTCTAGCTCTCTCGAAGGTTCACCTGATGAGTTTTCCAACGGCGAGGAATTGTGTGGAGT |
| | AATGACAATTAGCGATTCCCCACGGGAACAAGACAGTGAGTCCCAAACTCTCGATAAGGTCTACCA |
| | GATGAAAAGTAAGCCCAGGGGCTATTGCTTGATTATCAATAACCACAACTTTGCTAAGGCCCGGGA |
| | AAAAGTACCGAAACTCCACTCCATCCGGGATCGCAATGGTACTCACCTGGACGCTGGGGCGCTTAC |
| | TACCACCTTTGAAGAGCTGCATTTTGAGATAAAACCACACGACGACTGCACGGTTGAACAAATCTA |
| | TGAGATATTGAAAATCTATCAGCTTATGGATCATTCCAATATGGACTGTTTCATCTGCTGCATACTT |
| | AGCCACGGGGATAAAGGTATTATATACGGTACAGACGGTCAAGAGGCTCCGATATATGAGCTCACG |
| | TCACAATTTACTGGTCTCAAGTGCCCTAGTCTGGCAGGGAAGCCCAAGGTGTTCTTCATTCAGGCCT |
| | GTCAGGGCGATAATTATCAGAAAGGGATTCCTGTGGAGACGGACAGTGAAGAGCAACCGTACCTC |
| | GAGATGGATTTGTCCTCTCCGCAGACGCGATATATTCCGGATGAAGCCGATTTCCTGCTTGGCATGG |
| | CCACTGTGAACAACTGCGTCTCCTATCGGAATCCTGCAGAAGGTACGTGGTATATACAGTCTCTCTG |
| | TCAAAGTTTGAGGGAAAGGTGCCCGCGGGGAGATGATATCTTGACCATTTTGACGGAAGTGAACTA |
| | TGAAGTGAGTAACAAAGATGACAAGAAAAACATGGGTAAGCAGATGCCGCAACCGACTTTTACCC |
| | TGCGCAAAAAACTGGTCTTCCCTTCTGAT (SEQ ID NO: 340) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDILDYSFTGGAGRDIPPPQIEEACELPE |
| (H)-SynNotch | CQVDAGNKVCNLQCNNHACGWDGGDCSLNFNDPWKNCTQSLQCWKYFSDGHCDSQCNSAGCLFDGF |
| (TMD)-tCasp8 | DCQLTEGQCNPLYDQYCKDHFSDGHCDQGCNSAECEWDGLDCAEHVPERLAAGTLVLVVLLPPDQLR |
| (amino acid) | NNSFHFLRELSHVLHTNVVFKRDAQGQQMIFPYYGHEEELRKHPIKRSTVGWATSSLLPGTSGGRQRRE |
| | LDPMDIRGSIVYLEIDNRQCVQSSSQCFQSATDVAAFLGALASLGSLNIPYKIEAVKSEPVEPPLPSQLHL |
| | MYVAAAAFVLLFFVGCGVLLSRKRRRSESQTLDKVYQMKSKPRGYCLIINNHNFAKAREKVPKLHSIRD |
| | RNGTHLDAGALTTTFEELHFEIKPHDDCTVEQIYEILKIYQLMDHSNMDCFICCILSHGDKGIIYGTDGQE |
| | APIYELTSQFTGLKCPSLAGKPKVFFIQACQGDNYQKGIPVETDSEEQPYLEMDLSSPQTRYIPDEADFLL |
| | GMATVNNCVSYRNPAEGTWYIQSLCQSLRERCPRGDDILTILTEVNYEVSNKDDKKNMGKQMPQPTFT |
| | LRKKLVFPSD (SEQ ID NO: 255) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-SynNotch | ACTACCACCCCAGCACCCAGACCGCCAACTCCGGCTCCTACGATAGCGTCCCAACCTCTCTCTCTTA |
| (TMD)-tCasp8 | GGCCAGAGGCATGCCGCCCGGCAGCTGGGGGTGCGGTTCATACTCGGGGACTTGATTTCGCTTGTG |
| (nucleic acid) | ACATTTTGGATTATAGTTTCACCGGAGGCGCCGGACGCGACATACCTCCCCCGCAAATCGAGGAAG |
| | CGTGCGAACTCCCAGAGTGTCAAGTAGACGCCGGCAACAAGGTGTGCAACCTGCAATGCAACAACC |
| | ATGCCTGCGGATGGGATGGAGGGGACTGTAGTTTGAATTTCAACGACCCTTGGAAGAACTGCACGC |
| | AGTCTCTTCAATGCTGGAAATATTTCAGCGATGGACATTGTGACAGCCAGTGTAACAGCGCCGGAT |
| | GCTTGTTTGATGGCTTTGATTGTCAATTGACCGAGGGTCAGTGCAACCCGCTCTATGACCAATATTG |
| | TAAGGATCACTTTTCCGACGGGCACTGCGATCAGGGCTGCAATAGCGCGGAATGTGAATGGGACGG |
| | ATTGGATTGTGCAGAACATGTGCCCGAACGCCTCGCGGCAGGAACTCTGGTCCTCGTAGTCTTGTTG |
| | CCGCCCGACCAATTGCGGAACAACAGTTTCCACTTTCTGCGGGAATTGTCACATGTGCTGCATACAA |
| | ATGTCGTGTTTAAGCGCGATGCTCAGGGTCAACAAATGATATTCCCGTACTATGGTCACGAAGAGG |
| | AGCTTAGGAAGCACCCGATTAAGCGGTCTACGGTTGGATGGGCTACTTCCTCTCTTCTCCCGGGGAC |
| | GAGCGGGGGCAGACAAAGGCGGGAGCTCGACCCAATGGACATTAGAGGATCAATAGTGTACCTGG |
| | AGATTGACAATCGGCAATGCGTCCAATCTAGCAGTCAATGCTTTCAGTCTGCAACCGATGTCGCAG |
| | CCTTTCTGGGCGCCCTGGCCAGCCTCGGGTCCCTTAATATCCCGTATAAGATTGAGGCGGTTAAATC |
| | TGAACCTGTCGAACCTCCCCTCCCCTCTCAATTGCACCTCATGTACGTTGCTGCTGCTGCCTTTGTTC |
| | TGCTCTTCTTCGTAGGATGCGGGGTTCTTCTCTCCAGAAAACGACGGCGCAGTGAAAGCCAGACGC |
| | TCGATAAAGTTTACCAGATGAAGTCCAAACCACGGGGTTACTGTCTCATAATCAATAACCACAATTT |
| | CGCCAAGGCAAGAGAAAAAGTTCCTAAGCTCCATAGCATCAGAGACAGGAACGGGACTCACTTGG |
| | ACGCTGGGGCTCTGACTACCACTTTCGAGGAGTTGCACTTCGAGATTAAACCACATGACGACTGCA |
| | CAGTAGAGCAGATTTACGAAATACTCAAAATCTATCAACTTATGGATCATAGCAATATGGACTGCT |
| | TTATCTGTTGTATACTTTCCCACGGTGATAAGGGCATCATATACGGGACCGATGGTCAGGAAGCTCC |
| | AATATATGAACTCACAAGTCAGTTTACTGGTCTGAAGTGTCCATCATTGGCGGGGAAGCCCAAGGT |
| | CTTTTTTATTCAGGCCTGTCAAGGCGACAACTACCAAAAGGGCATTCCAGTAGAGACAGACTCAGA |
| | GGAACAGCCATATCTCGAGATGGATCTTTCCTCCCCTCAAACCAGATACATTCCAGATGAAGCAGA |
| | CTTCCTGTTGGGGATGGCTACTGTTAATAACTGTGTTTCATATCGCAACCCAGCGGAGGGAACATGG |
| | TACATACAGTCCTTGTGTCAAAGTCTGAGGGAACGATGCCCAAGGGGGGATGATATCCTGACTATC |
| | TTGACCGAGGTGAACTACGAGGTGTCCAACAAAGACGATAAAAAGAATATGGGGAAACAAATGCC |
| | TCAGCCAACTTTTACGCTTCGAAAAAAGCTCGTCTTCCCCAGCGAT (SEQ ID NO: 341) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDILDYSFTGGAGRDIPPPQIEEACELPE |
| (H)-SynNotch | CQVDAGNKVCNLQCNNHACGWDGGDCSLNFNDPWKNCTQSLQCWKYFSDGHCDSQCNSAGCLFDGF |
| (TMD)-tCasp8- | DCQLTEGQCNPLYDQYCKDHFSDGHCDQGCNSAECEWDGLDCAEHVPERLAAGTLVLVVLLPPDQLR |
| dimer | NNSFHFLRELSHVLHTNVVFKRDAQGQQMIFPYYGHEEELRKHPIKRSTVGWATSSLLPGTSGGRQRRE |
| (amino acid) | LDPMDIRGSIVYLEIDNRQCVQSSSQCFQSATDVAAFLGALASLGSLNIPYKIEAVKSEPVEPPLPSQLHL |
| | MYVAAAAFVLLFFVGCGVLLSRKRRRSESQTLDKVYQMKSKPRGYCLIINNHNFAKAREKVPKLHSIRD |
| | RNGTHLDAGALTTTFEELHFEIKPHDDCTVEQIYEILKIYQLMDHSNMDCFICCILSHGDKGIIYGTDGQE |
| | APIYELTSQFTGLKCPSLAGKPKVFFIQACQGDNYQKGIPVETDSEEQPYLEMDLSSPQTRYIPDEADFLL |
| | GMATVNNCVSYRNPAEGTWYIQSLCQSLRERCPRGDDILTILTEVNYEVSNKDDKKNMGKQMPCIVSM |
| | LRKKLVFPSD (SEQ ID NO: 256) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-SynNotch | ACTACCACCCCAGCACCCAGACCGCCAACTCCGGCTCCTACGATAGCGTCCCAACCTCTCTCTCTTA |
| (TMD)-tCasp8- | GGCCAGAGGCATGCCGCCCGGCAGCTGGGGGTGCGGTTCATACTCGGGGACTTGATTTCGCTTGTG |
| dimer | ACATTTTGGATTATAGTTTCACCGGAGGCGCCGGACGCGACATACCTCCCCCGCAAATCGAGGAAG |
| (nucleic acid) | CGTGCGAACTCCCAGAGTGTCAAGTAGACGCCGGCAACAAGGTGTGCAACCTGCAATGCAACAACC |
| | ATGCCTGCGGATGGGATGGAGGGGACTGTAGTTTGAATTTCAACGACCCTTGGAAGAACTGCACGC |
| | AGTCTCTTCAATGCTGGAAATATTTCAGCGATGGACATTGTGACAGCCAGTGTAACAGCGCCGGAT |
| | GCTTGTTTGATGGCTTTGATTGTCAATTGACCGAGGGTCAGTGCAACCCGCTCTATGACCAATATTG |
| | TAAGGATCACTTTTCCGACGGGCACTGCGATCAGGGCTGCAATAGCGCGGAATGTGAATGGGACGG |
| | ATTGGATTGTGCAGAACATGTGCCCGAACGCCTCGCGGCAGGAACTCTGGTCCTCGTAGTCTTGTTG |
| | CCGCCCGACCAATTGCGGAACAACAGTTTCCACTTTCTGCGGGAATTGTCACATGTGCTGCATACAA |
| | ATGTCGTGTTTAAGCGCGATGCTCAGGGTCAACAAATGATATTCCCGTACTATGGTCACGAAGAGG |
| | AGCTTAGGAAGCACCCGATTAAGCGGTCTACGGTTGGATGGGCTACTTCCTCTCTTCTCCCGGGGAC |
| | GAGCGGGGGCAGACAAAGGCGGGAGCTCGACCCAATGGACATTAGAGGATCAATAGTGTACCTGG |
| | AGATTGACAATCGGCAATGCGTCCAATCTAGCAGTCAATGCTTTCAGTCTGCAACCGATGTCGCAG |
| | CCTTTCTGGGCGCCCTGGCCAGCCTCGGGTCCCTTAATATCCCGTATAAGATTGAGGCGGTTAAATC |
| | TGAACCTGTCGAACCTCCCCTCCCCTCTCAATTGCACCTCATGTACGTTGCTGCTGCTGCCTTTGTTC |
| | TGCTCTTCTTCGTAGGATGCGGGGTTCTTCTCTCCAGAAAACGACGGCGCTCAGAATCACAAACTCT |
| | GGATAAGGTATATCAGATGAAGAGCAAACCGCGAGGATATTGCTTGATTATCAACAACCATAATTT |
| | CGCTAAAGCTAGAGAAAAAGTCCCAAAACTGCACTCCATTAGAGACAGGAATGGCACCCATTTGGA |
| | CGCGGGTGCGCTCACTACCACATTCGAAGAATTGCACTTTGAAATTAAGCCCCACGATGACTGCAC |
| | TGTGGAACAGATTTACGAGATACTGAAAATCTATCAACTTATGGACCACTCCAACATGGATTGTTTT |
| | ATATGCTGCATTTTGTCCCATGGTGATAAGGGAATCATATACGGAACAGATGGACAGGAAGCGCCA |
| | ATTTATGAACTTACCAGCCAGTTCACGGGACTTAAGTGTCCGAGCCTTGCTGGGAAGCCGAAGGTC |
| | TTTTTTATACAAGCGTGTCAAGGTGACAACTATCAGAAAGGAATTCCAGTCGAAACTGATTCTGAA |
| | GAGCAGCCATACCTGGAGATGGATCTCAGTTCTCCCCAGACCAGGTACATTCCCGATGAAGCGGAT |
| | TTTTTGCTCGGTATGGCAACAGTGAACAACTGTGTTTCTTACAGGAACCCGGCAGAAGGTACTTGGT |
| | ATATTCAAAGCTTGTGCCAATCTCTTCGGGAGCGGTGTCCGAGAGGAGACGACATCCTCACTATACT |
| | CACGGAAGTAAATTATGAGGTGAGTAACAAGGACGATAAAAAAAACATGGGAAAACAAATGCCCT |
| | GCATAGTTTCTATGTTGAGAAAAAAACTCGTTTTTCCTAGCGAC (SEQ ID NO: 342) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDILDYSFTGGAGRDIPPPQIEEACELPE |
| (H)-SynNotch | CQVDAGNKVCNLQCNNHACGWDGGDCSLNFNDPWKNCTQSLQCWKYFSDGHCDSQCNSAGCLFDGF |
| (TMD)-tBid15 | DCQLTEGQCNPLYDQYCKDHFSDGHCDQGCNSAECEWDGLDCAEHVPERLAAGTLVLVVLLPPDQLR |
| (amino acid) | NNSFHFLRELSHVLHTNVVFKRDAQGQQMIFPYYGHEEELRKHPIKRSTVGWATSSLLPGTSGGRQRRE |
| | LDPMDIRGSIVYLEIDNRQCVQSSSQCFQSATDVAAFLGALASLGSLNIPYKIEAVKSEPVEPPLPSQLHL |
| | MYVAAAAFVLLFFVGCGVLLSRKRRRGNRSSHSRLGRIEADSESQEDIIRNIARHLAQVGDSMDRSIPPG |
| | LVNGLALQLRNTSRSEEDRNRDLATALEQLLQAYPRDMEKEKTMLVLALLLAKKVASHTPSLLRDVFH |
| | TTVNFINQNLRTYVRSLARNGMD (SEQ ID NO: 257) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-SynNotch | ACTACCACCCCAGCACCCAGACCGCCAACTCCGGCTCCTACGATAGCGTCCCAACCTCTCTCTCTTA |
| (TMD)-tBid15 | GGCCAGAGGCATGCCGCCCGGCAGCTGGGGGTGCGGTTCATACTCGGGGACTTGATTTCGCTTGTG |
| (nucleic acid) | ACATTTTGGATTATAGTTTCACCGGAGGCGCCGGACGCGACATACCTCCCCCGCAAATCGAGGAAG |
| | CGTGCGAACTCCCAGAGTGTCAAGTAGACGCCGGCAACAAGGTGTGCAACCTGCAATGCAACAACC |
| | ATGCCTGCGGATGGGATGGAGGGGACTGTAGTTTGAATTTCAACGACCCTTGGAAGAACTGCACGC |
| | AGTCTCTTCAATGCTGGAAATATTTCAGCGATGGACATTGTGACAGCCAGTGTAACAGCGCCGGAT |
| | GCTTGTTTGATGGCTTTGATTGTCAATTGACCGAGGGTCAGTGCAACCCGCTCTATGACCAATATTG |
| | TAAGGATCACTTTTCCGACGGGCACTGCGATCAGGGCTGCAATAGCGCGGAATGTGAATGGGACGG |
| | ATTGGATTGTGCAGAACATGTGCCCGAACGCCTCGCGGCAGGAACTCTGGTCCTCGTAGTCTTGTTG |
| | CCGCCCGACCAATTGCGGAACAACAGTTTCCACTTTCTGCGGGAATTGTCACATGTGCTGCATACAA |
| | ATGTCGTGTTTAAGCGCGATGCTCAGGGTCAACAAATGATATTCCCGTACTATGGTCACGAAGAGG |
| | AGCTTAGGAAGCACCCGATTAAGCGGTCTACGGTTGGATGGGCTACTTCCTCTCTTCTCCCGGGGAC |
| | GAGCGGGGGCAGACAAAGGCGGGAGCTCGACCCAATGGACATTAGAGGATCAATAGTGTACCTGG |
| | AGATTGACAATCGGCAATGCGTCCAATCTAGCAGTCAATGCTTTCAGTCTGCAACCGATGTCGCAG |
| | CCTTTCTGGGCGCCCTGGCCAGCCTCGGGTCCCTTAATATCCCGTATAAGATTGAGGCGGTTAAATC |
| | TGAACCTGTCGAACCTCCCCTCCCCTCTCAATTGCACCTCATGTACGTTGCTGCTGCTGCCTTTGTTC |
| | TGCTCTTCTTCGTAGGATGCGGGGTTCTTCTCTCCAGAAAACGACGGCGCGGCAATCGAAGTTCTCA |
| | CTCACGCCTTGGACGGATCGAAGCAGATTCAGAAAGTCAAGAGGACATCATACGGAACATAGCCC |
| | GGCATTTGGCCCAAGTAGGGGATAGTATGGACCGGAGTATTCCACCTGGTCTTGTAAACGGTTTGG |
| | CTTTGCAGTTGAGAAACACCAGTAGGAGTGAAGAAGACCGCAACAGGGATCTCGCCACGGCCCTTG |
| | AGCAACTTTTGCAAGCATATCCAAGAGATATGGAAAAAGAAAAGACGATGTTGGTCCTTGCATTGC |
| | TTCTGGCAAAAAAGGTTGCGTCTCACACGCCTTCTTTGTTGCGCGATGTCTTTCACACTACGGTAAA |
| | TTTCATCAACCAGAACCTTCGGACTTACGTGCGGTCTCTTGCCCGAAACGGCATGGAT (SEQ ID NO: |
| | 343) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDILDYSFTGGAGRDIPPPQIEEACELPE |
| (H)-SynNotch | CQVDAGNKVCNLQCNNHACGWDGGDCSLNFNDPWKNCTQSLQCWKYFSDGHCDSQCNSAGCLFDGF |
| (TMD)- | DCQLTEGQCNPLYDQYCKDHFSDGHCDQGCNSAECEWDGLDCAEHVPERLAAGTLVLVVLLPPDQLR |
| Casp9wt | NNSFHFLRELSHVLHTNVVFKRDAQGQQMIFPYYGHEEELRKHPIKRSTVGWATSSLLPGTSGGRQRRE |
| (amino acid) | LDPMDIRGSIVYLEIDNRQCVQSSSQCFQSATDVAAFLGALASLGSLNIPYKIEAVKSEPVEPPLPSQLHL |
| | MYVAAAAFVLLFFVGCGVLLSRKRRRMDEADRRLLRRCRLRLVEELQVDQLWDALLSRELFRPHMIED |
| | IQRAGSGSRRDQARQLIIDLETRGSQALPLFISCLEDTGQDMLASFLRTNRQAAKLSKPTLENLTPVVLRP |
| | EIRKPEVLRPETPRPVDIGSGGFGDVGALESLRGNADLAYILSMEPCGHCLIINNVNFCRESGLRTRTGSNI |
| | DCEKLRRRFSSLHFMVEVKGDLTAKKMVLALLELAQQDHGALDCCVVVILSHGCQASHLQFPGAVYG |
| | TDGCPVSVEKIVNIFNGTSCPSLGGKPKLFFIQACGGEQKDHGFEVASTSPEDESPGSNPEPDATPFQEGL |
| | RTFDQLDAISSLPTPSDIFVSYSTFPGFVSWRDPKSGSWYVETLDDIFEQWAHSEDLQSLLLRVANAVSV |
| | KGIYKQMPGCFNFLRKKLFFKTS (SEQ ID NO: 258) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-SynNotch | ACTACCACCCCAGCACCCAGACCGCCAACTCCGGCTCCTACGATAGCGTCCCAACCTCTCTCTCTTA |
| (TMD)- | GGCCAGAGGCATGCCGCCCGGCAGCTGGGGGTGCGGTTCATACTCGGGGACTTGATTTCGCTTGTG |
| Casp9wt | ACATTTTGGATTATAGTTTCACCGGAGGCGCCGGACGCGACATACCTCCCCCGCAAATCGAGGAAG |
| (nucleic acid) | CGTGCGAACTCCCAGAGTGTCAAGTAGACGCCGGCAACAAGGTGTGCAACCTGCAATGCAACAACC |
| | ATGCCTGCGGATGGGATGGAGGGGACTGTAGTTTGAATTTCAACGACCCTTGGAAGAACTGCACGC |
| | AGTCTCTTCAATGCTGGAAATATTTCAGCGATGGACATTGTGACAGCCAGTGTAACAGCGCCGGAT |
| | GCTTGTTTGATGGCTTTGATTGTCAATTGACCGAGGGTCAGTGCAACCCGCTCTATGACCAATATTG |
| | TAAGGATCACTTTTCCGACGGGCACTGCGATCAGGGCTGCAATAGCGCGGAATGTGAATGGGACGG |
| | ATTGGATTGTGCAGAACATGTGCCCGAACGCCTCGCGGCAGGAACTCTGGTCCTCGTAGTCTTGTTG |
| | CCGCCCGACCAATTGCGGAACAACAGTTTCCACTTTCTGCGGGAATTGTCACATGTGCTGCATACAA |
| | ATGTCGTGTTTAAGCGCGATGCTCAGGGTCAACAAATGATATTCCCGTACTATGGTCACGAAGAGG |
| | AGCTTAGGAAGCACCCGATTAAGCGGTCTACGGTTGGATGGGCTACTTCCTCTCTTCTCCCGGGGAC |
| | GAGCGGGGGCAGACAAAGGCGGGAGCTCGACCCAATGGACATTAGAGGATCAATAGTGTACCTGG |
| | AGATTGACAATCGGCAATGCGTCCAATCTAGCAGTCAATGCTTTCAGTCTGCAACCGATGTCGCAG |
| | CCTTTCTGGGCGCCCTGGCCAGCCTCGGGTCCCTTAATATCCCGTATAAGATTGAGGCGGTTAAATC |
| | TGAACCTGTCGAACCTCCCCTCCCCTCTCAATTGCACCTCATGTACGTTGCTGCTGCTGCCTTTGTTC |
| | TGCTCTTCTTCGTAGGATGCGGGGTTCTTCTCTCCAGAAAACGACGGCGCATGGACGAGGCTGATCG |
| | CAGATTGCTTAGGCGATGTCGGTTGAGACTGGTAGAAGAGTTGCAAGTCGATCAATTGTGGGATGC |
| | GCTTCTGTCTAGGGAGCTGTTTCGGCCGCACATGATCGAAGACATCCAAAGGGCAGGTTCAGGATC |
| | ACGCCGAGATCAAGCGAGACAGCTGATAATCGATCTGGAAACGCGAGGATCACAGGCGTTGCCTCT |
| | CTTCATCAGTTGTCTGGAGGATACGGGGCAGGACATGCTCGCTTCCTTTCTGCGAACCAATCGCCAA |
| | GCCGCAAAGCTCAGCAAACCGACACTTGAAAACCTGACGCCGGTTGTGCTGAGGCCAGAAATACG |
| | GAAACCAGAGGTCCTGAGACCCGAAACACCACGACCAGTAGACATTGGGAGCGGGGGTTTCGGAG |
| | ATGTAGGCGCATTGGAGTCCCTTCGAGGCAATGCGGATCTTGCATATATTCTTAGTATGGAACCTTG |
| | TGGACACTGTCTCATTATCAACAATGTGAATTTCTGCCGCGAGTCAGGACTGCGAACCCGGACAGG |
| | CTCAAACATTGATTGTGAGAAACTTCGACGCAGATTCTCATCCTTGCATTTTATGGTAGAGGTTAAG |
| | GGGGATCTTACAGCGAAGAAAATGGTTCTTGCTCTGCTCGAACTTGCACAGCAAGACCACGGGGCA |
| | CTGGATTGTTGCGTAGTCGTGATTCTTTCCCATGGATGTCAAGCGTCTCATCTCCAGTTTCCGGGCGC |
| | GGTCTATGGTACGGACGGCTGCCCCGTTTCAGTAGAAAAGATAGTCAACATTTTCAACGGCACCTC |
| | ATGCCCGTCCTTGGGTGGCAAACCCAAGTTGTTTTTTATCCAAGCGTGCGGTGGAGAACAGAAAGA |
| | TCATGGGTTTGAAGTGGCGTCCACATCACCCGAAGATGAAAGCCCTGGGAGCAACCCTGAACCAGA |
| | CGCGACTCCTTTTCAAGAGGGCTTGCGGACTTTTGACCAGTTGGACGCCATATCATCATTGCCGACG |
| | CCGTCTGATATATTCGTCTCCTATAGTACATTCCCTGGTTTTGTCTCTTGGAGGGACCCCAAAAGCG |
| | GCTCTTGGTATGTTGAAACATTGGATGATATTTTTGAACAATGGGCGCACAGTGAGGATCTTCAGAG |
| | CTTGCTTCTGCGAGTCGCTAACGCAGTTTCCGTAAAGGGAATATACAAACAGATGCCCGGATGCTTT |
| | AATTTCCTCCGCAAAAAGCTGTTTTTTAAGACTTCT (SEQ ID NO: 344) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDILDYSFTGGAGRDIPPPQIEEACELPE |
| (H)-SynNotch | CQVDAGNKVCNLQCNNHACGWDGGDCSLNFNDPWKNCTQSLQCWKYFSDGHCDSQCNSAGCLFDGF |
| (TMD)-tCasp9 | DCQLTEGQCNPLYDQYCKDHFSDGHCDQGCNSAECEWDGLDCAEHVPERLAAGTLVLVVLLPPDQLR |
| (amino acid) | NNSFHFLRELSHVLHTNVVFKRDAQGQQMIFPYYGHEEELRKHPIKRSTVGWATSSLLPGTSGGRQRRE |
| | LDPMDIRGSIVYLEIDNRQCVQSSSQCFQSATDVAAFLGALASLGSLNIPYKIEAVKSEPVEPPLPSQLHL |
| | MYVAAAAFVLLFFVGCGVLLSRKRRRGFGDVGALESLRGNADLAYILSMEPCGHCLIINNVNFCRESGL |
| | RTRTGSNIDCEKLRRRFSSLHFMVEVKGDLTAKKMVLALLELARQDHGALDCCVVVILSHGCQASHLQ |
| | FPGAVYGTDGCPVSVEKIVNIFNGTSCPSLGGKPKLFFIQACGGEQKDHGFEVASTSPEDESPGSNPEPDA |
| | TPFQEGLRTFDQLDAISSLPTPSDIFVSYSTFPGFVSWRDPKSGSWYVETLDDIFEQWAHSEDLQSLLLRV |
| | ANAVSVKGIYKQMPGCFNFLRKKLFFKTS (SEQ ID NO: 259) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-SynNotch | ACTACCACCCCAGCACCCAGACCGCCAACTCCGGCTCCTACGATAGCGTCCCAACCTCTCTCTCTTA |
| (TMD)-tCasp9 | GGCCAGAGGCATGCCGCCCGGCAGCTGGGGGTGCGGTTCATACTCGGGGACTTGATTTCGCTTGTG |
| (nucleic acid) | ACATTTTGGATTATAGTTTCACCGGAGGCGCCGGACGCGACATACCTCCCCCGCAAATCGAGGAAG |
| | CGTGCGAACTCCCAGAGTGTCAAGTAGACGCCGGCAACAAGGTGTGCAACCTGCAATGCAACAACC |
| | ATGCCTGCGGATGGGATGGAGGGGACTGTAGTTTGAATTTCAACGACCCTTGGAAGAACTGCACGC |
| | AGTCTCTTCAATGCTGGAAATATTTCAGCGATGGACATTGTGACAGCCAGTGTAACAGCGCCGGAT |
| | GCTTGTTTGATGGCTTTGATTGTCAATTGACCGAGGGTCAGTGCAACCCGCTCTATGACCAATATTG |
| | TAAGGATCACTTTTCCGACGGGCACTGCGATCAGGGCTGCAATAGCGCGGAATGTGAATGGGACGG |
| | ATTGGATTGTGCAGAACATGTGCCCGAACGCCTCGCGGCAGGAACTCTGGTCCTCGTAGTCTTGTTG |
| | CCGCCCGACCAATTGCGGAACAACAGTTTCCACTTTCTGCGGGAATTGTCACATGTGCTGCATACAA |
| | ATGTCGTGTTTAAGCGCGATGCTCAGGGTCAACAAATGATATTCCCGTACTATGGTCACGAAGAGG |
| | AGCTTAGGAAGCACCCGATTAAGCGGTCTACGGTTGGATGGGCTACTTCCTCTCTTCTCCCGGGGAC |
| | GAGCGGGGGCAGACAAAGGCGGGAGCTCGACCCAATGGACATTAGAGGATCAATAGTGTACCTGG |
| | AGATTGACAATCGGCAATGCGTCCAATCTAGCAGTCAATGCTTTCAGTCTGCAACCGATGTCGCAG |
| | CCTTTCTGGGCGCCCTGGCCAGCCTCGGGTCCCTTAATATCCCGTATAAGATTGAGGCGGTTAAATC |
| | TGAACCTGTCGAACCTCCCCTCCCCTCTCAATTGCACCTCATGTACGTTGCTGCTGCTGCCTTTGTTC |
| | TGCTCTTCTTCGTAGGATGCGGGGTTCTTCTCTCCAGAAAACGACGGCGCGGATTTGGGGATGTAGG |
| | AGCACTCGAATCTCTTAGGGGGAATGCTGATTTGGCTTATATCCTTTCTATGGAACCATGCGGACAC |
| | TGTCTGATAATAAATAATGTCAATTTCTGCCGGGAAAGCGGATTGCGAACCCGAACGGGCTCAAAC |
| | ATCGATTGTGAGAAGCTGCGACGACGGTTTAGTTCCCTTCATTTTATGGTCGAAGTCAAAGGCGATC |
| | TTACCGCCAAGAAAATGGTTTTGGCACTCTTGGAGCTTGCGCGACAAGATCACGGGGCATTGGATT |
| | GTTGTGTTGTGGTCATCTTGAGCCATGGTTGTCAAGCGTCACACTTGCAGTTTCCCGGTGCCGTTTAT |
| | GGGACTGATGGCTGTCCAGTATCCGTTGAAAAGATCGTCAATATATTTAATGGCACATCTTGTCCTT |
| | CCCTGGGCGGGAAGCCCAAACTCTTTTTTATTCAGGCCTGCGGTGGTGAACAGAAAGATCACGGTT |
| | TTGAAGTTGCATCTACCTCTCCAGAAGACGAATCCCCTGGGAGCAACCCTGAACCTGACGCGACTC |
| | CATTCCAAGAGGGGCTTCGGACGTTCGACCAACTCGACGCAATATCAAGTTTGCCGACACCGAGCG |
| | ACATATTCGTCTCATACAGCACATTCCCCGGTTTCGTATCTTGGAGAGATCCAAAGTCAGGGAGTTG |
| | GTATGTCGAGACTTTGGATGATATTTTTGAACAATGGGCGCACTCCGAGGATCTTCAGAGTCTTCTC |
| | CTGCGGGTGGCGAATGCGGTGAGCGTGAAAGGAATTTACAAGCAGATGCCAGGGTGCTTTAACTTC |
| | CTCCGGAAAAAGCTGTTTTTCAAAACTAGC (SEQ ID NO: 345) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDILDYSFTGGAGRDIPPPQIEEACELPE |
| (H)-SynNotch | CQVDAGNKVCNLQCNNHACGWDGGDCSLNFNDPWKNCTQSLQCWKYFSDGHCDSQCNSAGCLFDGF |
| (TMD)-tCasp9- | DCQLTEGQCNPLYDQYCKDHFSDGHCDQGCNSAECEWDGLDCAEHVPERLAAGTLVLVVLLPPDQLR |
| dimer | NNSFHFLRELSHVLHTNVVFKRDAQGQQMIFPYYGHEEELRKHPIKRSTVGWATSSLLPGTSGGRQRRE |
| (amino acid) | LDPMDIRGSIVYLEIDNRQCVQSSSQCFQSATDVAAFLGALASLGSLNIPYKIEAVKSEPVEPPLPSQLHL |
| | MYVAAAAFVLLFFVGCGVLLSRKRRRGFGDVGALESLRGNADLAYILSMEPCGHCLIINNVNFCRESGL |
| | RTRTGSNIDCEKLRRRFSSLHFMVEVKGDLTAKKMVLALLELAQQDHGALDCCVVVILSHGCQASHLQ |
| | FPGAVYGTDGCPVSVEKIVNIFNGTSCPSLGGKPKLFFIQACGGEQKDHGFEVASTSPEDESPGSNPEPDA |
| | TPFQEGLRTFDQLDAISSLPTPSDIFVSYSTFPGFVSWRDPKSGSWYVETLDDIFEQWAHSEDLQSLLLRV |
| | ANAVSVKGIYKQMPCIVSMLRKKLFFKTS (SEQ ID NO: 260) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-SynNotch | ACTACCACCCCAGCACCCAGACCGCCAACTCCGGCTCCTACGATAGCGTCCCAACCTCTCTCTCTTA |
| (TMD)-tCasp9- | GGCCAGAGGCATGCCGCCCGGCAGCTGGGGGTGCGGTTCATACTCGGGGACTTGATTTCGCTTGTG |
| dimer | ACATTTTGGATTATAGTTTCACCGGAGGCGCCGGACGCGACATACCTCCCCCGCAAATCGAGGAAG |
| (nucleic acid) | CGTGCGAACTCCCAGAGTGTCAAGTAGACGCCGGCAACAAGGTGTGCAACCTGCAATGCAACAACC |
| | ATGCCTGCGGATGGGATGGAGGGGACTGTAGTTTGAATTTCAACGACCCTTGGAAGAACTGCACGC |
| | AGTCTCTTCAATGCTGGAAATATTTCAGCGATGGACATTGTGACAGCCAGTGTAACAGCGCCGGAT |
| | GCTTGTTTGATGGCTTTGATTGTCAATTGACCGAGGGTCAGTGCAACCCGCTCTATGACCAATATTG |
| | TAAGGATCACTTTTCCGACGGGCACTGCGATCAGGGCTGCAATAGCGCGGAATGTGAATGGGACGG |
| | ATTGGATTGTGCAGAACATGTGCCCGAACGCCTCGCGGCAGGAACTCTGGTCCTCGTAGTCTTGTTG |
| | CCGCCCGACCAATTGCGGAACAACAGTTTCCACTTTCTGCGGGAATTGTCACATGTGCTGCATACAA |
| | ATGTCGTGTTTAAGCGCGATGCTCAGGGTCAACAAATGATATTCCCGTACTATGGTCACGAAGAGG |
| | AGCTTAGGAAGCACCCGATTAAGCGGTCTACGGTTGGATGGGCTACTTCCTCTCTTCTCCCGGGGAC |
| | GAGCGGGGGCAGACAAAGGCGGGAGCTCGACCCAATGGACATTAGAGGATCAATAGTGTACCTGG |
| | AGATTGACAATCGGCAATGCGTCCAATCTAGCAGTCAATGCTTTCAGTCTGCAACCGATGTCGCAG |
| | CCTTTCTGGGCGCCCTGGCCAGCCTCGGGTCCCTTAATATCCCGTATAAGATTGAGGCGGTTAAATC |
| | TGAACCTGTCGAACCTCCCCTCCCCTCTCAATTGCACCTCATGTACGTTGCTGCTGCTGCCTTTGTTC |
| | TGCTCTTCTTCGTAGGATGCGGGGTTCTTCTCTCCAGAAAACGACGGCGCGGATTTGGTGATGTCGG |
| | TGCCCTCGAAAGTCTTAGAGGCAACGCTGATCTGGCGTATATTTTGAGCATGGAACCATGTGGACA |
| | TTGTCTTATCATCAATAACGTAAATTTTTGTAGGGAAAGTGGCCTTAGGACTAGGACAGGTTCCAAC |
| | ATCGACTGCGAAAAACTGCGGAGGAGATTCTCTTCCCTCCATTTTATGGTGGAAGTCAAAGGCGAT |
| | CTCACCGCAAAGAAGATGGTACTCGCATTGCTCGAACTTGCCCAACAGGACCATGGAGCTTTGGAC |
| | TGCTGTGTTGTAGTTATACTGTCACATGGTTGCCAGGCAAGCCACCTCCAGTTTCCTGGGGCGGTAT |
| | ATGGGACCGATGGTTGCCCCGTTTCCGTAGAGAAGATAGTAAATATCTTCAACGGAACGAGCTGTC |
| | CGTCCCTCGGGGGCAAGCCAAAGCTTTTTTTCATTCAAGCCTGTGGCGGCGAGCAAAAAGACCACG |
| | GATTCGAGGTGGCATCAACGTCCCCTGAAGATGAGAGTCCGGGCAGCAATCCCGAACCGGATGCTA |
| | CCCCATTCCAAGAAGGTCTTAGGACATTCGACCAACTGGACGCTATATCTAGTTTGCCAACTCCTTC |
| | AGATATCTTTGTAAGTTACTCTACGTTTCCTGGCTTCGTAAGTTGGAGAGATCCTAAATCTGGAAGT |
| | TGGTATGTGGAAACGTTGGATGACATTTTTGAACAATGGGCTCATAGTGAAGACCTTCAGTCACTCC |
| | TGCTTCGCGTGGCAAATGCAGTGTCAGTTAAGGGTATCTACAAACAGATGCCGTGCATCGTATCCAT |
| | GCTTCGGAAAAAGCTGTTCTTTAAAACAAGC (SEQ ID NO: 346) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD45 | GGGGSTTTLSPSGSAVISTTTIATTPSKPTCDEKYANITVDYLYNKETKLFTAKLNVNENVECGNNTCTN |
| (H)-CD8 | NEVHNLTECKNASVSISHNSCTAPDKTLILDVPPGVEKFQLHDCTQVEKADTTICLKWKNIETFTCDTQN |
| (TMD)-PD1 | ITYRFQCGNMIFDNKEIKLENLEPEHEYKCDSEILYNNHKFTNASKIIKTDFGSPGEPQIIFCRSEAAHQGVI |
| (amino acid) | TWNPPQRSFHNFTLCYIKETEKDCLNLDKNLIKYDLQNLKPYTKYVLSLHAYIIAKVQRNGSAAMCHFT |
| | TKSAPPSQVWNMTVSMTSDNSMHVKCRPPRDRNGPHERYHLEVEAGNTLVRNESHKNCDFRVKDLQY |
| | STDYTFKAYFHNGDYPGEPFILHHSTSYNSKIYIWAPLAGTCGVLLLSLVITCSRAARGTIGARRTGQPLK |
| | EDPSAVPVFSVDYGELDFQWREKTPEPPVPCVPEQTEYATIVFPSGMGTSSPARRGSADGPRSAQPLRPE |
| | DGHCSWPL (SEQ ID NO: 261) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD45 | FROM TABLE 6--]] |
| (H)-CD8 | GGTGGAGGCGGTTCCACAACGACCTTATCTCCATCCGGTTCCGCAGTCATTAGCACCACGACCATA |
| (TMD)-PD1 | GCTACCACGCCAAGTAAGCCAACTTGCGATGAGAAATACGCTAACATCACCGTGGATTATCTGTAT |
| (nucleic acid) | AATAAAGAAACGAAGTTGTTCACAGCTAAGCTCAATGTAAACGAGAACGTGGAGTGCGGGAATAA |
| | TACTTGCACTAACAACGAAGTCCATAATCTCACTGAGTGCAAGAATGCTTCTGTGTCCATTTCTCAT |
| | AACTCTTGCACTGCCCCAGATAAGACACTGATACTGGATGTGCCACCAGGCGTGGAAAAGTTTCAG |
| | CTTCACGACTGTACCCAGGTCGAGAAAGCTGACACGACTATCTGTCTGAAATGGAAGAACATAGAA |
| | ACGTTTACATGCGATACACAGAACATTACCTATCGATTCCAATGCGGGAACATGATTTTCGACAATA |
| | AGGAAATTAAACTGGAGAATCTGGAGCCTGAACATGAGTACAAATGTGACTCCGAAATCCTGTACA |
| | ATAATCACAAGTTCACAAATGCGAGCAAAATTATCAAGACTGACTTCGGATCACCAGGCGAACCCC |
| | AGATTATTTTCTGTCGCAGTGAGGCTGCACATCAGGGAGTCATAACATGGAACCCGCCACAGAGAA |
| | GTTTCCATAACTTTACACTCTGTTACATAAAGGAGACTGAGAAAGATTGCCTTAACTTAGATAAGAA |
| | CCTGATCAAGTACGACCTTCAGAACTTGAAACCGTATACCAAGTATGTCCTTTCCCTGCACGCCTAC |
| | ATAATCGCCAAAGTGCAGCGAAATGGATCTGCTGCCATGTGCCATTTCACTACGAAATCTGCACCA |
| | CCTTCCCAGGTGTGGAACATGACAGTGTCTATGACCAGTGACAATTCAATGCACGTTAAATGTAGA |
| | CCACCGAGGGATAGAAATGGTCCACACGAGAGGTACCATTTAGAGGTAGAGGCAGGAAATACCTT |
| | AGTGAGAAATGAGAGCCACAAGAACTGCGACTTCAGAGTGAAAGACTTACAGTACTCCACTGATTA |
| | TACATTTAAAGCTTATTTCCATAATGGAGATTACCCCGGCGAACCTTTTATACTCCACCACTCTACT |
| | AGCTACAACTCCAAAATATACATTTGGGCTCCATTGGCGGGCACGTGCGGAGTTCTGCTTCTCAGTT |
| | TGGTTATTACCTGTAGTCGCGCTGCAAGAGGTACCATTGGTGCGAGGAGGACCGGACAACCTTTGA |
| | AGGAGGACCCAAGTGCGGTACCTGTTTTTTCCGTCGACTACGGCGAGCTGGATTTCCAGTGGCGAG |
| | AAAAGACACCAGAACCACCGGTTCCTTGTGTACCTGAGCAGACGGAGTATGCGACAATCGTCTTCC |
| | CGAGCGGAATGGGCACGAGTTCCCCCGCCAGAAGGGGGTCTGCCGACGGACCTAGGAGCGCACAA |
| | CCACTTAGACCCGAAGACGGCCATTGTTCCTGGCCACTG (SEQ ID NO: 347) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITGGGGS |
| (H)-CD8 | GGGGSMVRWFHRDLSGLDAETLLKGRGVHGSFLARPSRKNQGDFSLSVRVGDQVTHIRIQNSGDFYDL |
| (TMD)-G4S- | YGGEKFATLTELVEYYTQQQGVLQDRDGTIIHLKYPLNCSDPTSERWYHGHMSGGQAETLLQAKGEPW |
| SHP 1 | TFLVRESLSQPGDFVLSVLSDQPKAGPGSPLRVTHIKVMCEGGRYTVGGLETFDSLTDLVEHFKKTGIEE |
| (amino acid) | ASGAFVYLRQPYYATRVNAADIENRVLELNKKQESEDTAKAGFWEEFESLQKQEVKNLHQRLEGQRPE |
| | NKGKNRYKNILPFDHSRVILQGRDSNIPGSDYINANYIKNQLLGPDENAKTYIASQGCLEATVNDFWQM |
| | AWQENSRVIVMTTREVEKGRNKCVPYWPEVGMQRAYGPYSVTNCGEHDTTEYKLRTLQVSPLDNGDL |
| | IREIWHYQYLSWPDHGVPSEPGGVLSFLDQINQRQESLPHAGPIIVHCSAGIGRTGTIIVIDMLMENISTKG |
| | LDCDIDIQKTIQMVRAQRSGMVQTEAQYKFIYVAIAQFIETTKKKLEVLQSQKGQESEYGNITYPPAMKN |
| | AHAKASRTSSKHKEDVYENLHTKNKREEKVKKQRSADKEKSKGSLKRK (SEQ ID NO: 262) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-G4S- | GGCCAGAGGCATGCCGCCCGGCAGCTGGGGGTGCGGTTCATACTCGGGGACTTGATTTCGCTTGTG |
| SHP1 | ACATATACATTTGGGCTCCATTGGCGGGCACGTGCGGAGTTCTGCTTCTCAGTTTGGTTATTACCGG |
| (nucleic acid) | AGGTGGTGGGTCAGGTGGAGGCGGAAGTATGGTACGATGGTTCCATCGCGACTTGTCCGGACTGGA |
| | CGCTGAGACCCTCCTGAAAGGGCGGGGTGTGCACGGCAGTTTCCTTGCACGACCCTCCAGAAAGAA |
| | TCAAGGCGACTTTAGTCTTTCAGTAAGGGTTGGTGATCAGGTCACACACATTAGAATCCAAAATTCA |
| | GGTGACTTCTATGATTTGTATGGCGGTGAGAAATTTGCAACGCTCACCGAACTTGTTGAATATTACA |
| | CCCAGCAACAGGGAGTACTTCAGGACCGCGACGGTACAATAATTCACCTTAAGTACCCCTTGAATT |
| | GTTCAGATCCTACGTCCGAGAGATGGTATCACGGACACATGAGTGGAGGACAAGCTGAAACCCTCC |
| | TCCAAGCAAAGGGGGAACCCTGGACTTTTTTGGTCAGAGAGAGTCTTAGCCAGCCGGGTGATTTTG |
| | TTCTTAGTGTGCTGTCCGATCAACCGAAAGCGGGGCCTGGTTCACCTTTGCGCGTGACCCATATTAA |
| | AGTTATGTGTGAAGGAGGTCGATATACCGTTGGGGGCCTTGAAACATTCGATAGTCTGACGGATTT |
| | GGTCGAACACTTCAAAAAAACAGGGATTGAAGAGGCCTCCGGCGCTTTTGTCTATCTTAGACAACC |
| | CTATTATGCGACGAGGGTGAACGCTGCGGATATCGAGAATAGAGTTCTGGAGCTGAATAAAAAGCA |
| | AGAATCAGAGGACACCGCCAAAGCTGGTTTCTGGGAGGAGTTCGAGAGTTTGCAGAAGCAGGAAG |
| | TTAAAAACCTCCACCAGAGGTTGGAAGGGCAGAGACCGGAAAACAAGGGTAAGAACCGCTATAAA |
| | AATATCTTGCCCTTCGATCACTCTCGGGTCATACTGCAGGGTAGAGACAGTAACATTCCAGGCAGTG |
| | ATTACATCAACGCTAACTATATAAAAAATCAGCTTCTGGGCCCAGATGAAAATGCAAAGACCTATA |
| | TTGCGAGTCAGGGCTGTCTGGAGGCCACGGTTAATGACTTCTGGCAGATGGCATGGCAAGAAAACA |
| | GTAGGGTAATCGTCATGACAACTAGAGAAGTTGAGAAAGGACGGAACAAGTGTGTTCCTTACTGGC |
| | CCGAGGTAGGCATGCAGCGGGCGTACGGGCCCTACAGTGTTACCAACTGCGGCGAACATGATACCA |
| | CAGAGTATAAATTGCGAACACTCCAAGTGTCACCACTGGATAACGGCGACCTTATCAGGGAGATCT |
| | GGCACTACCAATACCTGTCCTGGCCTGATCACGGCGTACCATCCGAACCGGGGGGAGTGCTTAGCT |
| | TTCTGGATCAGATAAACCAAAGACAGGAATCTCTGCCCCATGCAGGTCCGATCATCGTCCATTGTTC |
| | CGCGGGTATAGGTCGGACCGGAACGATTATCGTAATTGACATGTTGATGGAAAACATCTCTACGAA |
| | AGGGCTCGATTGCGATATAGACATCCAGAAGACCATACAAATGGTACGGGCACAAAGATCAGGCA |
| | TGGTCCAGACCGAAGCCCAATACAAATTCATCTACGTCGCTATTGCCCAGTTCATTGAAACAACGA |
| | AAAAGAAGTTGGAGGTTCTCCAATCCCAGAAGGGACAAGAGTCTGAGTACGGTAACATAACTTATC |
| | CGCCTGCCATGAAAAACGCTCATGCAAAGGCGAGCCGGACTAGTAGTAAGCACAAAGAAGACGTT |
| | TACGAGAATCTGCATACCAAAAATAAGCGGGAGGAAAAAGTAAAAAAACAACGATCAGCTGATAA |
| | AGAGAAATCTAAAGGCTCATTGAAGAGAAAA (SEQ ID NO: 348) |
| CD8 (SP)- | MALPVTALLLPLALLLHAARP (SEQ ID NO: 292) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDIYIWAPLAGTCGVLLLSLVITGGGGS |
| (H)-CD8 | GGGGSMSAIQAAWPSGTECIAKYNFHGTAEQDLPFCKGDVLTIVAVTKDPNWYKAKNKVGREGIIPAN |
| (TMD)-CSK | YVQKREGVKAGTKLSLMPWFHGKITREQAERLLYPPETGLFLVRESTNYPGDYTLCVSCDGKVEHYRI |
| (amino acid) | MYHASKLSIDEEVYFENLMQLVEHYTSDADGLCTRLIKPKVMEGTVAAQDEFYRSGWALNMKELKLL |
| | QTIGKGEFGDVMLGDYRGNKVAVKCIKNDATAQAFLAEASVMTQLRHSNLVQLLGVIVEEKGGLYIVT |
| | EYMAKGSLVDYLRSRGRSVLGGDCLLKFSLDVCEAMEYLEGNNFVHRDLAARNVLVSEDNVAKVSDF |
| | GLTKEASSTQDTGKLPVKWTAPEALREKKFSTKSDVWSFGILLWEIYSFGRVPYPRIPLKDVVPRVEKGY |
| | KMDAPDGCPPAVYEVMKNCWHLDAAMRPSFLQLREQLEHIKTHELHL (SEQ ID NO: 263) |
| CD8 (SP)- | ATGGCCCTTCCCGTGACTGCGCTCCTCCTGCCTCTTGCCCTCCTTCTTCATGCTGCTCGCCCG (SEQ ID |
| iCAR Antigen | NO: 327) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD8 | ACTACCACCCCAGCACCCAGACCGCCAACTCCGGCTCCTACGATAGCGTCCCAACCTCTCTCTCTTA |
| (TMD)-CSK | GGCCAGAGGCATGCCGCCCGGCAGCTGGGGGTGCGGTTCATACTCGGGGACTTGATTTCGCTTGTG |
| (nucleic acid) | ACATATACATTTGGGCTCCATTGGCGGGCACGTGCGGAGTTCTGCTTCTCAGTTTGGTTATTACCGG |
| | AGGTGGTGGGTCAGGTGGAGGCGGAAGTATGTCCGCCATACAGGCAGCATGGCCCAGCGGAACGG |
| | AATGTATCGCTAAATACAACTTTCACGGCACCGCGGAGCAAGATCTTCCGTTCTGTAAAGGTGACG |
| | TTCTTACCATAGTGGCGGTTACTAAGGACCCAAATTGGTATAAAGCTAAGAACAAGGTCGGGCGCG |
| | AGGGCATAATCCCAGCAAACTATGTCCAGAAGAGGGAAGGCGTGAAAGCGGGAACCAAATTGAGC |
| | CTCATGCCGTGGTTCCACGGCAAGATTACACGCGAACAAGCGGAGCGACTGCTTTATCCACCAGAG |
| | ACGGGTCTTTTCCTGGTCCGGGAGTCCACGAATTATCCGGGGGACTATACACTTTGTGTAAGCTGTG |
| | ACGGCAAGGTCGAACACTACAGGATCATGTACCACGCTAGTAAACTTAGCATAGATGAGGAGGTAT |
| | ATTTTGAGAATCTCATGCAGCTTGTAGAACATTACACAAGTGACGCGGATGGATTGTGTACTCGATT |
| | GATTAAGCCAAAGGTTATGGAAGGTACTGTCGCAGCCCAAGACGAGTTTTACCGATCCGGCTGGGC |
| | TCTGAACATGAAAGAGCTCAAACTTCTCCAGACCATCGGTAAGGGTGAGTTTGGCGACGTAATGTT |
| | GGGTGATTACCGCGGAAATAAGGTGGCTGTAAAATGTATCAAGAACGATGCAACAGCTCAAGCCTT |
| | CCTGGCTGAGGCGTCAGTGATGACACAACTTAGACACTCAAACTTGGTCCAATTGCTCGGAGTTATC |
| | GTCGAAGAGAAAGGCGGCCTCTATATTGTTACTGAGTATATGGCTAAAGGCTCCCTTGTGGACTATC |
| | TTCGATCTCGAGGCAGGTCTGTTCTCGGTGGGGATTGCTTGTTGAAATTCAGTCTTGATGTTTGCGA |
| | AGCTATGGAATATCTGGAAGGCAACAATTTTGTCCATAGGGACCTGGCCGCCCGGAATGTGTTGGT |
| | TTCAGAAGATAACGTGGCCAAGGTGTCCGACTTTGGTCTGACAAAAGAGGCTAGCTCCACTCAGGA |
| | CACTGGGAAGTTGCCGGTAAAATGGACGGCCCCTGAAGCATTGAGAGAGAAAAAATTCTCTACTAA |
| | GTCCGACGTGTGGTCTTTTGGCATTCTTCTTTGGGAAATCTACTCATTCGGAAGGGTCCCGTACCCTC |
| | GCATTCCGCTTAAGGACGTTGTCCCTCGGGTCGAAAAGGGCTACAAGATGGACGCTCCTGACGGAT |
| | GCCCGCCTGCGGTCTACGAGGTAATGAAAAATTGCTGGCATCTTGATGCTGCAATGCGCCCAAGTTT |
| | CTTGCAATTGCGAGAGCAACTGGAACACATAAAGACCCACGAGCTCCATCTC (SEQ ID NO: 349) |
| MARS (SP)- | MARSPAQLLGLLLLWLSGARC (SEQ ID NO: 97) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDFWVLVVVGGVLACYSLLVTVAFIIF |
| (H)-CD28 | WVITQGLAVSTISSFKRGRKKLLYIFKQPFMRPVQTTQEEDGCSCRFPEEEEGGCELRVKFSRSADAPAY |
| (TMD)- | QQGQNQLYNELNLGRREEYDVLDKRRGRDPEMGGKPRRKNPQEGLYNELQKDKMAEAYSEIGMKGE |
| ADAM17 | RRRGKGHDGLYQGLSTATKDTYDALHMQALPPR (SEQ ID NO: 264) |
| Cleavage Site- | |
| CD28-CD3z | |
| (amino acid) | |
| MARS (SP)- | ATGGCCAGATCCCCGGCACAACTGCTCGGACTCCTCCTGTTGTGGTTGAGCGGGGCCCGCTGT (SEQ |
| iCAR Antigen | ID NO: 378) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD28 | ACTACCACCCCAGCACCCAGACCGCCAACTCCGGCTCCTACGATAGCGTCCCAACCTCTCTCTCTTA |
| (TMD)- | GGCCAGAGGCATGCCGCCCGGCAGCTGGGGGTGCGGTTCATACTCGGGGACTTGATTTCGCTTGTG |
| ADAM17 | ACTTTTGGGTGCTGGTGGTGGTTGGTGGAGTCCTGGCTTGCTATTCCTTGCTAGTAACAGTGGCCTT |
| Cleavage Site- | CATCATCTTTTGGGTCATCACCCAGGGCCTGGCCGTGAGCACCATCAGCAGCTTCAAACGGGGCAG |
| CD28-CD3z | AAAGAAACTCCTGTATATATTCAAACAACCATTTATGCGACCAGTACAAACTACTCAAGAGGAAGA |
| (nucleic acid) | TGGCTGTAGCTGCCGATTTCCAGAAGAAGAAGAAGGAGGATGTGAACTGAGAGTGAAGTTCAGCA |
| | GGAGCGCAGACGCCCCCGCGTACCAGCAGGGCCAGAACCAGCTCTATAACGAGCTCAATCTAGGA |
| | CGAAGAGAGGAGTACGATGTTTTGGACAAGCGACGTGGCCGGGACCCTGAGATGGGGGGAAAGCC |
| | GAGAAGGAAGAACCCTCAGGAAGGCCTGTACAATGAACTGCAGAAAGATAAGATGGCGGAGGCCT |
| | ACAGTGAGATTGGGATGAAAGGCGAGCGCCGGAGGGGCAAGGGGCACGATGGCCTTTACCAGGGA |
| | CTCAGTACAGCCACCAAGGACACCTACGACGCCCTTCACATGCAGGCCCTGCCCCCTCGCTAA (SEQ |
| | ID NO: 350) |
| MARS (SP)- | MARSPAQLLGLLLLWLSGARC (SEQ ID NO: 97) |
| iCAR Antigen | [[--INSERT AMINO ACID SEQUENCE OF iCAR ANTIGEN BINDING DOMAIN SELECTED |
| Binding | FROM TABLE 5--]] |
| Domain-CD8 | TTTPAPRPPTPAPTIASQPLSLRPEACRPAAGGAVHTRGLDFACDFWVLVVVGGVLACYSLLVTVAFIIF |
| (H)-CD28 | WVGGGGSGGGGSGGGGSNTCKLLVVADHRFYRYMGRGEESTTTNYLIELIDRVDDIYRNTSWDNAGF |
| (TMD)-G43S | KGYGIQIEQIRILKSPQEVKPGEKHYNMAKSYPNEEKDAWDVKMLLEQFSFDIAEEASKVCLAHLFTYQ |
| Linker- | DFDMGTLGLAYVGSPRANSHGGVCPKAYYSPVGKKNIYLNSGLTSTKNYGKTILTKEADLVTTHELGH |
| ADAM17 | NFGAEHDPDGLAECAPNEDQGGKYVMYPIAVSGDHENNKMFSNCSKQSIYKTIESKAQECFQERS (SEQ |
| Protease | ID NO: 265) |
| Domain | |
| (amino acid) | |
| MARS (SP)- | ATGGCCAGATCCCCGGCACAACTGCTCGGACTCCTCCTGTTGTGGTTGAGCGGGGCCCGCTGT (SEQ |
| iCAR Antigen | ID NO: 378) |
| Binding | [[--INSERT NUCLEOTIDE SEQUENCE OF iCAR ANTIGEN BINDING DOMAIN SELECTED |
| Domain-CD8 | FROM TABLE 6--]] |
| (H)-CD28 | ACCACGACACCGGCTCCTCGGCCACCGACACCGGCACCAACAATTGCTTCCCAGCCACTCAGTCTG |
| (TMD)-G43S | CGGCCGGAGGCATGTCGGCCTGCGGCTGGCGGAGCTGTTCACACGAGGGGCCTCGATTTTGCATGC |
| Linker- | GATTTCTGGGTTCTTGTAGTAGTTGGCGGAGTCCTTGCTTGCTACAGTTTGCTTGTGACGGTAGCATT |
| ADAM17 | CATCATATTCTGGGTAGGCGGCGGGGGATCTGGAGGTGGTGGCAGCGGTGGGGGCGGTAGTAATAC |
| Protease | GTGTAAGCTCCTTGTGGTAGCAGACCATAGGTTCTACAGATATATGGGACGAGGAGAGGAAAGCAC |
| Domain | TACGACAAACTATTTGATCGAACTTATAGATAGAGTCGACGACATCTACCGAAACACGTCATGGGA |
| (nucleic acid) | CAATGCAGGCTTCAAGGGATACGGAATACAGATAGAGCAGATACGGATTCTGAAAAGTCCGCAGG |
| | AAGTGAAACCGGGGGAGAAACATTATAATATGGCGAAATCATATCCAAACGAGGAGAAAGATGCG |
| | TGGGATGTAAAAATGCTCTTGGAACAGTTTAGTTTTGACATTGCTGAGGAAGCCAGCAAAGTGTGC |
| | CTCGCTCACCTGTTTACTTATCAAGATTTTGATATGGGCACCCTGGGTCTTGCATATGTAGGGTCTCC |
| | CAGAGCAAACTCCCACGGGGGCGTATGTCCAAAAGCTTACTACAGCCCAGTAGGAAAAAAGAATA |
| | TATACCTGAACAGCGGGCTGACTAGTACCAAAAATTACGGAAAAACTATTCTCACGAAGGAGGCCG |
| | ACTTGGTAACTACTCATGAACTGGGGCATAACTTCGGCGCCGAGCACGACCCGGACGGACTTGCAG |
| | AGTGCGCCCCGAACGAGGACCAAGGTGGCAAATATGTCATGTACCCAATCGCCGTTTCAGGGGATC |
| | ATGAGAACAATAAGATGTTCAGCAACTGTTCCAAACAATCCATTTATAAAACGATAGAAAGCAAAG |
| | CCCAGGAATGTTTTCAGGAAAGATCA (SEQ ID NO: 351) |
| MARS (SP)- | MARSPAQLLGLLLLWLSGARC (SEQ ID NO: 97) |
| iCAR Antigen | [[--INSERT AMINO ACID SEQUENCE OF iCAR ANTIGEN BINDING DOMAIN SELECTED |
| Binding | FROM TABLE 5--]] |
| Domain- | VLEVSDHQVLNDAEVAALLENFSSSYDYGENESDSCCTSPPCPQDFSLNFDRAFLPALYSLLFLLGLLGN |
| CXC3R GPCR | GAVAAVLLSRRTALSSTDTFLLHLAVADTLLVLTLPLWAVDAAVQWVFGSGLCKVAGALFNINFYAGA |
| (H)-CXC3R | LLLACISFDRYLNIVHAVSLSKMLKKRSPLTTGVYVKMPPTEPECEKQFQPYFIPINPARVTLTCLAVWG |
| GPCR (TMD)- | LCLLFALPDFIFLSAHHDERLNATHCQYNFPQVGRTALRVLQLVAGFLLPLLVMAYCYAHILAVLLVLT |
| CTLA4-TIGIT- | RKKKALRIHSVEGDLRRKSAGQEEWSPSAPSPPGSCVQAEAAPAGLCGEQRGEDCAELHDYFNVLSYRS |
| PD1 | LGNCSFFTETGQRRLRAMRLVVVVVVAFALCWTPYHLVVLVDILMDLGALARNCGRESRVDVAKSVT |
| (amino acid) | SGLGYMHCCLNPLLYAFVCSRAARGTIGARRTGQPLKEDPSAVPVFSVDYGELDFQWREKTPEPPVPCV |
| | PEQTEYATIVFPSGMGTSSPARRGSADGPRSAQPLRPEDGHCSWPL (SEQ ID NO: 352) |
| MARS (SP)- | ATGGCCAGATCTCCTGCTCAACTGCTGGGACTGCTGCTGCTGTGGCTTAGCGGAGCCAGATGC (SEQ |
| iCAR Antigen | ID NO: 98) |
| Binding | [[--INSERT NUCLEOTIDE SEQUENCE OF iCAR ANTIGEN BINDING DOMAIN SELECTED |
| Domain- | FROM TABLE 6--]] |
| CXC3R GPCR | GTCCTTGAGGTGTCCGACCACCAGGTCCTGAACGATGCTGAGGTGGCAGCGCTGTTGGAGAACTTC |
| (H)-CXC3R | AGTTCATCCTACGATTACGGAGAGAACGAGTCTGATAGTTGTTGTACCAGTCCCCCCTGCCCCCAGG |
| GPCR (TMD)- | ATTTTTCCCTTAATTTCGACAGGGCTTTCCTCCCCGCACTGTATTCCCTTCTGTTTCTTTTAGGGCTGT |
| CTLA4-TIGIT- | TAGGCAATGGCGCCGTGGCGGCTGTCCTTCTGAGTCGGCGCACAGCCCTCTCCAGTACGGATACCTT |
| PD1 | TCTCCTGCACCTGGCGGTGGCCGACACGCTGTTGGTCCTGACTCTTCCACTCTGGGCCGTTGATGCC |
| (nucleic acid) | GCAGTCCAATGGGTTTTTGGCTCCGGTCTGTGCAAGGTAGCGGGAGCTCTGTTCAATATCAACTTCT |
| | ACGCTGGAGCTCTGCTGCTGGCCTGCATCAGCTTCGATAGATATCTGAACATCGTCCATGCAGTCTC |
| | ACTCTCCAAAATGCTGAAAAAGCGCTCTCCCCTTACCACCGGCGTCTATGTCAAAATGCCACCTACT |
| | GAACCTGAATGCGAGAAGCAGTTCCAACCTTATTTTATCCCCATTAATCCAGCCAGAGTAACACTG |
| | ACATGTCTCGCTGTCTGGGGTCTGTGCCTGCTTTTCGCCCTGCCTGACTTCATTTTCCTGTCTGCTCA |
| | CCACGACGAGCGTCTGAATGCCACCCACTGCCAGTATAATTTCCCTCAAGTAGGTCGGACCGCCCTT |
| | CGAGTTTTGCAGCTGGTGGCCGGTTTCTTATTGCCCTTACTGGTGATGGCTTACTGCTACGCACACA |
| | TCTTGGCTGTGCTTCTCGTTTTGACTAGAAAGAAGAAAGCCCTCAGAATCCATTCTGTGGAAGGTGA |
| | CCTCAGGAGAAAATCAGCTGGACAGGAGGAATGGAGCCCCAGTGCTCCCTCACCCCCAGGAAGCT |
| | GTGTCCAGGCAGAAGCTGCACCTGCTGGGCTCTGTGGAGAGCAGCGGGGAGAGGACTGTGCCGAG |
| | CTGCATGACTACTTCAATGTCCTGAGTTACAGAAGCCTGGGTAACTGCAGCTTCTTCACAGAGACTG |
| | GTCAGAGGAGGCTGAGGGCCATGAGACTCGTTGTCGTCGTGGTTGTTGCTTTCGCCTTATGCTGGAC |
| | CCCATACCATTTAGTTGTGCTGGTGGATATCCTGATGGACCTGGGCGCCCTCGCACGGAACTGTGGC |
| | AGGGAATCAAGAGTGGACGTCGCTAAATCCGTGACCTCCGGACTGGGTTATATGCACTGTTGCCTC |
| | AACCCTCTGCTCTACGCCTTTGTGTGTAGTCGCGCTGCAAGAGGTACCATTGGTGCGAGGAGGACC |
| | GGACAACCTTTGAAGGAGGACCCAAGTGCGGTACCTGTTTTTTCCGTCGACTACGGCGAGCTGGAT |
| | TTCCAGTGGCGAGAAAAGACACCAGAACCACCGGTTCCTTGTGTACCTGAGCAGACGGAGTATGCG |
| | ACAATCGTCTTCCCGAGCGGAATGGGCACGAGTTCCCCCGCCAGAAGGGGGTCTGCCGACGGACCT |
| | AGGAGCGCACAACCACTTAGACCCGAAGACGGCCATTGTTCCTGGCCACTG (SEQ ID NO: 353) |

TABLE 5

| | |
|---|---|
| | SEQ ID |
| Sequence | NO |

iCAR Antigen Binding Domains (Amino Acid Sequences)

| | | |
|---|---|---|
| SR25022-P03- | EVQLLESGGGLVQPGGSLRLSCAASGLTFSSYAMGWFRQAPGKEREVVSVISGTGRSTYYA | 354 |
| V2M-R4P-G01 | DSVKGRFTISRDNSKNTLYLQMNSLKPEDTAVYYCAAGISQYYGDVLLFDYWGQGTQVTV | |
| | SS | |
| SR25022-P03- | EVQLLESGGGLVQPGGSLRLSCAASGFSFSSYAMGWYRQAPGKEREGVSAISTTGGSSYYA | 355 |
| V2M-R3P-A04 | DSVKGRFTISRDNSKNTLYLQMNSLKPEDTAVYYCAQVLDVADRSEYPLWGQGTQVTVSS | |
| SR25022-P03- | EVQLLESGGGLVQPGGSLRLSCAASGYTFSNYAMGWFRQAPGKEREFVSVISYSGGSTYYA | 356 |
| V2M-R3P-A12 | DSVKGRFTISRDNSKNTLYLQMNSLKPEDTAVYYCARRVAYQFRVYEYWGQGTQVTVSS | |
| SR25022-P03- | EVQLLESGGGLVQPGGSLRLSCAASGFTFSSYAMGWYRQAPGKEREGVSAISGSGRSTYYA | 357 |
| V2M-R3P-B04 | DSVKGRFTISRDNSKNTLYLQMNSLKPEDTAVYYCAQVLSLSDEEGPYWGQGTQVTVSS | |
| SR25022-P03- | EVQLLESGGGLVQPGGSLRLSCAASGYTFSSRAMGWYRQAPGKEREFVSVITGTGRTYYA | 358 |
| V2M-R4P-F07 | DSVKGRFTISRDNSKNTLYLQMNSLKPEDTAVYYCARIVLSSYSGFADYWGQGTQVTVSS | |
| SR25022-P01- | EVQLLESGGGLVQPGGSLRLSCAASGYTFDNYGMGWFRQAPGKERELVSAISGSGSGTYY | 359 |
| V2M-R3B-H04 | ADSVKGRFTISRDNSKNTLYLQMNSLKPEDTAVYYCAADVAYLAVGWTGYEYWGQGTQ | |
| | VTVSS | |
| SR25022-P01- | EVQLLESGGGLVQPGGSLRLSCAASGSTLDYYAMGWFRQAPGKEREFVSSISGTGGRTYYA | 360 |
| V2M-R2B-A06 | DSVKGRFTISRDNSKNTLYLQMNSLKPEDTAVYYCAADLGLYIVWELADYWGQGTQVTV | |
| | SS | |
| SR25022-P02- | EVQLLESGGGLVQPGGSLRLSCAASGLIFYIDAMGWFRQAPGKERELVSAISGSGGSIYYAD | 361 |
| V2S-R2B-C12 | SVKGRFTISRDNSKNTLYLQMNSLKPEDTAVYYCAADYYLGSEFDYWGQGTQVTVSS | |
| SR25022-P04- | EVQLLESGGGLVQPGGSLRLSCAASGYTFSDYYMGWYRQAPGKERELVSAISGSGSETNY | 362 |
| V2S-R3P-C12 | ADSVKGRFTISRDNSKNTLYLQMNSLKPEDTAVYYCAVDVLYWYAADYWGQGTLTEYW | |
| | GQGTQVTVSS | |
| SR25022-P02- | EVQLLESGGGLVQPGGSLRLSCAASGSIFYIYAIGWYRQAPGKERELVSVISGSGGSTYYAD | 363 |
| V2S-R3B-H04 | SVKGRFTISRDNSKNTLYLQMNSLKPEDTAVYYCAVDAYTDFDYWGQGTQVTVSS | |

TABLE 6

| | |
|---|---|
| | SEQ ID |
| Sequence | NO |

iCAR Antigen Binding Domains (Nucleic Acid Sequences)

| | | |
|---|---|---|
| SR25022-P03- | GAGGTACAACTTTTGGAGTCAGGCGGTGGACTGGTACAACCGGGTGGTTCATTGCGTTT | 364 |
| V2M-R4P-G01 | GAGCTGCGCTGCCTCTGGTTTGACTTTCAGCTCTTACGCTATGGGTTGGTTCCGCCAAGC | |
| | GCCGGGCAAAGAACGCGAGGTCGTATCCGTTATAAGCGGTACGGGGAGAAGTACTTAC | |
| | TACGCGGACTCGGTAAAAGGCCGTTTTACGATCAGTCGTGATAATTCCAAGAATACCTT | |
| | GTACCTGCAAATGAATAGCCTTAAGCCCGAAGACACAGCGGTGTATTATTGTGCCGCCG | |
| | GCATATCCCAGTACTACGGCGATGTGCTGTTATTTGACTATTGGGGCCAGGGTACCCAG | |
| | GTGACGGTCTCGAGC | |
| SR25022-P03- | GAGGTACAACTTTTGGAGTCAGGCGGTGGACTGGTACAACCGGGTGGTTCATTGCGTTT | 365 |
| V2M-R3P-A04 | GAGCTGCGCTGCCTCTGGTTTTTCATTTTCCTCTTACGCTATGGGCTGGTATCGCCAAGC | |
| | GCCGGGCAAAGAACGCGAGGGCGTCTCCGCAATATCCACGACCGGGGGCAGCAGCTAT | |
| | TACGCGGACTCGGTAAAAGGCCGTTTTACGATCAGTCGTGATAATTCCAAGAATACCTT | |
| | GTACCTGCAAATGAATAGCCTTAAGCCCGAAGACACAGCGGTGTATTATTGTGCCCAG | |
| | GTTTTAGATGTAGCTGATCGGTCAGAGTACCCGTTGTGGGGCCAGGGTACCCAGGTGAC | |
| | GGTCTCGAGC | |
| SR25022-P03- | GAGGTACAACTTTTGGAGTCAGGCGGTGGACTGGTACAACCGGGTGGTTCATTGCGTTT | 366 |
| V2M-R3P-A12 | GAGCTGCGCTGCCTCTGGTTATACTTTTAGCAACTACGCTATGGGATGGTTCCGCCAAG | |
| | CGCCGGGCAAAGAACGCGAGTTTGTATCCGTAATCTCATACAGCGGGGGATCTACCTA | |
| | CTACGCGGACTCGGTAAAAGGCCGTTTTACGATCAGTCGTGATAATTCCAAGAATACCT | |
| | TGTACCTGCAAATGAATAGCCTTAAGCCCGAAGACACAGCGGTGTATTATTGTGCCAGA | |
| | AGAGTTGCTTATCAATTTCGGGTATACGAATACTGGGGCCAGGGTACCCAGGTGACGGT | |
| | CTCGAGC | |
| SR25022-P03- | GAGGTACAACTTTTGGAGTCAGGCGGTGGACTGGTACAACCGGGTGGTTCATTGCGTTT | 367 |
| V2M-R3P-B04 | GAGCTGCGCTGCCTCTGGTTTTACATTTTCCAGTTACGCAATGGGATGGTATCGCCAAG | |
| | CGCCGGGCAAAGAACGCGAGGGTGTTTCCGCAATATCAGGTAGTGGACGCTCCACCTA | |
| | TTACGCGGACTCGGTAAAAGGCCGTTTTACGATCAGTCGTGATAATTCCAAGAATACCT | |
| | TGTACCTGCAAATGAATAGCCTTAAGCCCGAAGACACAGCGGTGTATTATTGTGCCCAA | |
| | GTGCTTTCCCTTTCCGATGAGGAAGGCCCTTATTGGGGCCAGGGTACCCAGGTGACGGT | |
| | CTCGAGC | |
| SR25022-P03- | GAGGTACAACTTTTGGAGTCAGGCGGTGGACTGGTACAACCGGGTGGTTCATTGCGTTT | 368 |
| V2M-R4P-F07 | GAGCTGCGCTGCCTCTGGTTATACTTTCAGCAGTCGGGCTATGGGCTGGTATCGCCAAG | |
| | CGCCGGGCAAAGAACGCGAGTTTGTCAGCGTTATAACCGGTACCGGCCGGACGTACTA | |
| | CGCGGACTCGGTAAAAGGCCGTTTTACGATCAGTCGTGATAATTCCAAGAATACCTTGT | |
| | ACCTGCAAATGAATAGCCTTAAGCCCGAAGACACAGCGGTGTATTATTGTGCCCGAATT | |
| | GTGCTGTCCTCCTATTCCGGCTTCGCCGATTATTGGGGCCAGGGTACCCAGGTGACGGT | |
| | CTCGAGC | |
| SR25022-P01- | GAGGTACAACTTTTGGAGTCAGGCGGTGGACTGGTACAACCGGGTGGTTCATTGCGTTT | 369 |
| V2M-R3B-H04 | GAGCTGCGCTGCCTCTGGTTACACTTTTGACAACTATGGGATGGGCTGGTTCCGCCAAG | |
| | CGCCGGGCAAAGAACGCGAGCTGGTATCAGCAATTAGCGGATCAGGGAGTGGTACGTA | |
| | CTACGCGGACTCGGTAAAAGGCCGTTTTACGATCAGTCGTGATAATTCCAAGAATACCT | |
| | TGTACCTGCAAATGAATAGCCTTAAGCCCGAAGACACAGCGGTGTATTATTGTGCCGCA | |
| | GATGTGGCTTATCTGGCAGTCGGCTGGACGGGCTATGAATACTGGGGCCAGGGTACCC | |
| | AGGTGACGGTCTCGAGC | |
| SR25022-P01- | GAGGTACAACTTTTGGAGTCAGGCGGTGGACTGGTACAACCGGGTGGTTCATTGCGTTT | 370 |
| V2M-R2B-A06 | GAGCTGCGCTGCCTCTGGTTCCACGCTTGACTACTATGCGATGGGATGGTTCCGCCAAG | |
| | CGCCGGGCAAAGAACGCGAGTTTGTTTCCTCCATCTCTGGGACCGGTGGTCGTACTTAT | |
| | TACGCGGACTCGGTAAAAGGCCGTTTTACGATCAGTCGTGATAATTCCAAGAATACCTT | |
| | GTACCTGCAAATGAATAGCCTTAAGCCCGAAGACACAGCGGTGTATTATTGTGCCGCTG | |
| | ACTTGGGCCTGTACATAGTCTGGGAGCTGGCCGACTACTGGGGCCAGGGTACCCAGGT | |
| | GACGGTCTCGAGC | |
| SR25022-P02- | GAGGTACAACTTTTGGAGTCAGGCGGTGGACTGGTACAACCGGGTGGTTCATTGCGTTT | 371 |
| V2S-R2B-C12 | GAGCTGCGCTGCCTCTGGTCTGATATTCTATATCGACGCAATGGGTTGGTTTCGCCAAG | |
| | CGCCGGGCAAAGAACGCGAGTTGGTTTCTGCGATTTCCGGCTCTGGAGGAAGTATCTAT | |
| | TACGCGGACTCGGTAAAAGGCCGTTTTACGATCAGTCGTGATAATTCCAAGAATACCTT | |
| | GTACCTGCAAATGAATAGCCTTAAGCCCGAAGACACAGCGGTGTATTATTGTGCCGCG | |
| | GACTATTACTTGGGGAGTGAGTTTGACTACTGGGGCCAGGGTACCCAGGTGACGGTCTC | |
| | GAGC | |
| SR25022-P04- | GAGGTACAACTTTTGGAGTCAGGCGGTGGACTGGTACAACCGGGTGGTTCATTGCGTTT | 372 |
| V2S-R3P-C12 | GAGCTGCGCTGCCTCTGGTTATACGTTTAGTGATTACTATATGGGTTGGTATCGCCAAG | |
| | CGCCGGGCAAAGAACGCGAGTTAGTGTCAGCTATTTCTGGGTCAGGTTCCGAGACCAA | |
| | TTACGCGGACTCGGTAAAAGGCCGTTTTACGATCAGTCGTGATAATTCCAAGAATACCT | |
| | TGTACCTGCAAATGAATAGCCTTAAGCCCGAAGACACAGCGGTGTATTATTGTGCCGTC | |
| | GATGTTCTGTACTGGTATGCCGCCGACTACTGGGGCCAGGGTACCCTGACGGAATATTG | |
| | GGGCCAGGGTACCCAGGTGACGGTCTCGAGC | |
| SR25022-P02- | GAGGTACAACTTTTGGAGTCAGGCGGTGGACTGGTACAACCGGGTGGTTCATTGCGTTT | 373 |
| V2S-R3B-H04 | GAGCTGCGCTGCCTCTGGTAGTATTTTTTATATTTATGCTATTGGCTGGTATCGCCAAGC | |
| | GCCGGGCAAAGAACGCGAGCTGGTTTCTGTTATTTCCGGGTCGGGTGGCTCGACCTATT | |
| | ACGCGGACTCGGTAAAAGGCCGTTTTACGATCAGTCGTGATAATTCCAAGAATACCTTG | |
| | TACCTGCAAATGAATAGCCTTAAGCCCGAAGACACAGCGGTGTATTATTGTGCCGTTGA | |
| | TGCTTACACCGACTTTGATTACTGGGGCCAGGGTACCCAGGTGACGGTCTCGAGC | |

In some aspects, the present disclosure provides an induced pluripotent stem cell (iPSC) or a derivative cell thereof comprising one or more exogenous polynucleotides encoding a chimeric antigen receptor (CAR) comprising an antigen binding domain targeting a Nectin4 antigen. In some embodiments, the one or more exogenous polynucleotides further encode one or more inhibitory CARs (iCAR). In some embodiments, the one or more iCARs comprise at least one antigen binding domain targeting an antigen. In some embodiments, the antigen targeted by the at least one antigen binding domain of the iCAR is independently selected from the group consisting of Adrenoceptor Beta 2 (ADRB2), Aquaporin 4 (AQP4), Claudin 10 (CLDN10B), Desmocollin (DSC) 1, DSC3, Desmoglein (DSG) 1, DSG3, Glycerophosphodiester Phosphodiesterase Domain Containing 2 (GDPD2), Hydroxycarboxylic Acid Receptor 3 (HCAR3), Lymphocyte Antigen 6 Family Member D (LY6D), and V-Set And Immunoglobulin Domain Containing 8 (VSIG8).

In some embodiments, the iCAR comprises a signal peptide. A signal peptide is a short amino acid sequence that is included in the design of chimeric antigen receptors (CARs) to facilitate proper processing and targeting of the engineered protein. The signal peptide guides appropriate localization and display of the CAR on the cell surface, enabling it to recognize cancer cells and initiate immune responses. Signal peptides used in CARs are often derived from antibodies or other surface proteins that naturally contain such targeting sequences. Typically, they are attached to the N-terminus of the CAR construct, at the beginning of the protein sequence, and can be between 15 and 30 amino acids long with a central hydrophobic region flanked by positively charged residues. Any signal peptide known to a person of skill in the art may be used in combination with engineered iPSCs, or derivative cells thereof, of the present disclosure. However, different signal peptide sequences can be tested to optimize CAR expression and function. In some embodiments, the signal peptide of the iCAR comprises a CD8 signal peptide, a GMCSFR signal peptide, a MARS signal peptide, or an IgK signal peptide or variant thereof. In some embodiments, the signal peptide of the iCAR comprises an amino acid sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 97 and 292. In some embodiments, the signal peptide of the iCAR is encoded by a polynucleotide sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 98 and 327.

In some embodiments, the iCAR comprises an extracellular domain comprising a binding domain that specifically binds at least one antigen binding domain each targeting one or more selected from the group consisting of Adrenoceptor Beta 2 (ADRB2), Aquaporin 4 (AQP4), Claudin 10 (CLDN10B), Desmocollin (DSC) 1, DSC3, Desmoglein (DSG) 1, DSG3, Glycerophosphodiester Phosphodiesterase Domain Containing 2 (GDPD2), Hydroxycarboxylic Acid Receptor 3 (HCAR3), Lymphocyte Antigen 6 Family Member D (LY6D), V-Set And Immunoglobulin Domain Containing 8 (VSIG8). For example, the iCAR can comprise an extracellular domain comprising a first binding domain that specifically binds to ADRB2 and a second binding domain that binds to DSG1. In another example, the iCAR can comprise an extracellular domain comprising a first binding domain that specifically binds to DSC1 and a second binding domain that binds to HCAR3. In some embodiments, the iCAR comprises an extracellular domain comprising a binding domain that specifically binds Adrenoceptor Beta 2 (ADRB2), Aquaporin 4 (AQP4), Claudin 10 (CLDN10B), Desmocollin (DSC) 1, DSC3, Desmoglein (DSG) 1, DSG3, Glycerophosphodiester Phosphodiesterase Domain Containing 2 (GDPD2), Hydroxycarboxylic Acid Receptor 3 (HCAR3), Lymphocyte Antigen 6 Family Member D (LY6D), V-Set, or Immunoglobulin Domain Containing 8 (VSIG8). For example, the iCAR comprises an extracellular domain comprising a binding domain that specifically binds DSG1. In another example, the iCAR comprises an extracellular domain comprising a binding domain that specifically binds AQP4. In some embodiments, the extracellular domain of the iCAR comprises an amino acid sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 354-363. In some embodiments, at least a portion of the extracellular domain of the iCAR is encoded by a polynucleotide sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 364-373.

In some embodiments, the iCAR comprises a hinge region. The hinge domain links the CAR's recognition and functional elements, with its structural flexibility and length enabling antigen-binding capabilities and spatial orientation. The hinge provides structural flexibility between the target recognition domain and the cell, which enables free rotation and orientation of the antigen binding site. Generally, the hinge domain connects the antigen-binding motif (usually the scFv) to the transmembrane region of the CAR, and are about 12-60 amino acids long (with longer hinges providing more flexibility). In some embodiments, the hinge region of the iCAR is selected from the group consisting of a CD28 hinge region, a CD45 hinge region, a G4S-CD45 hinge region, a CD8 hinge region, and a CXC3R GPCR hinge region. In some embodiments, the hinge region of the iCAR comprises an amino acid sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 21, 22, 288, 289, and 321. In some embodiments, the hinge region of the iCAR is encoded by a polynucleotide having at least 60%, 65%, 70%, 75%, 80%, 85%, 90%, 91%, 92%, 93%, 94%, 95%, 96%, 97%, 98%, 99% or 100% sequence identity to one of SEQ ID NOs: 315-318, 320, and 322.

In some embodiments, the iCAR comprises one or more transmembrane domains. The transmembrane domain anchors the CAR in the cell membrane and transduces signals from antigen binding to cell activation via its connection to the cytoplasmic signaling sequences. Often derived from CD3-zeta, CD4, CD8 or CD28 proteins, a transmembrane domain can comprise hydrophobic amino acid sequences that span the lipid bilayer, have between about 20 to 30 amino acids in length, and form an alpha helical structure that integrates into the membrane with charged residues on both ends that anchor the helix. In some embodiments, a transmembrane domain can enable CAR dimerization or multimerization which can amplify signaling. Modifying transmembrane properties (e.g., hydrophobicity, length, flexibility and/or dimerization capability) can help optimize CAR expression and function. In some embodiments, the one or more transmembrane domains of the iCAR are independently selected from the group consisting of a CD28 transmembrane domain, a CD8 transmembrane domain, a PD1 transmembrane domain, a SynNotch transmembrane domain, and a CXC3R GPCR. In some embodiments, the transmembrane domain of the iCAR comprises an amino acid sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 23, 24, 290, 291, 323, and 325. In some embodiments, the transmembrane domain of the iCAR is encoded by a polynucleotide sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 324, 326, and 374-377.

In some embodiments, the iCAR comprises an intracellular signaling domain. The intracellular signaling domain(s) initiate and control the CAR engineered cell response through ITAM and costimulatory interactions that can be optimized to improve therapeutic benefit. Specifically, the intracellular signaling domain is the region of the CAR that initiates cell activation after the CAR binds its target antigen. Generally, intracellular signalling domains can comprise immunoreceptor tyrosine-based activation motifs (ITAMs) that mediate signal transduction, and often include CD3-zeta and/or costimulatory domains like CD28 or 4-1BB. When the CAR binds its antigen, ITAM tyrosines are phosphorylated, initiating the cell activation cascade through enzymes like ZAP70, leading to transcription factor activation. Inclusion of costimulatory signaling domains (e.g., CD28, 4-1BB) with CD3-zeta can provide synergistic signals to enhance cell activation, cytokine production, proliferation and persistence. Varying the combination and order of ITAM and costimulatory domains enables tuning of CAR signaling strength, balancing potency and safety. In some embodiments, the intracellular signaling domain of the iCAR comprises one or more of a PD1 intracellular domain, an LIRB1 intracellular domain, a TIGIT a CTLA4 intracellular domain, a CSK*(YSSV) intracellular domain, a KIR2DL1 intracellular domain, a DR1 intracellular domain, a Casp8 wt intracellular domain, a tCasp8 intracellular domain, a tCasp8-dimer intracellular domain, a tBid15 intracellular domain, a Casp9 wt intracellular domain, a tCasp9 intracellular domain, a tCasp9-dimer intracellular domain, a SHP1 intracellular domain, a (G4S)2-SHP1 intracellular domain, a CSK intracellular domain, a (G4S)2-CSK intracellular domain, an ADAM17 cleavage site, a CD28 intracellular domain, a CD3ζ intracellular domain, a G4S3 linker, an ADAM 17 protease domain, and a (G4S)3-ADAM 17 protease domain. In some embodiments, the intracellular signaling domain of the iCAR comprises an amino acid sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 6, 8, and 267-287. In some embodiments, the intracellular signaling domain of the iCAR is encoded by a polynucleotide sequence having at least 60%, 65%, 70%, 75%, 80%, 85%, 90%, 91%, 92%, 93%, 94%, 95%, 96%, 97%, 98%, 99% or 100% sequence identity to one of SEQ ID NOs: 266, 293-318, 320, and 322. In some embodiments, the iCAR comprises a co-stimulatory domain. In some embodiments, the co-stimulatory domain of the iCAR is selected from the group consisting of a CD28 signaling domain, a 41BB signaling domain, and a DAP10 signaling domain.

### III. Artificial Cell Death Polypeptide

According to embodiments of the application, an iPSC or a derivative cell thereof may comprise a second exogenous polynucleotide encoding an artificial cell death polypeptide.

As used herein, the term “artificial cell death polypeptide” refers to an engineered protein designed to prevent potential toxicity or otherwise adverse effects of a cell therapy. The artificial cell death polypeptide could mediate induction of apoptosis, inhibition of protein synthesis, DNA replication, growth arrest, transcriptional and post-transcriptional genetic regulation and/or antibody-mediated depletion. In some instance, the artificial cell death polypeptide is activated by an exogenous molecule, e.g. an antibody, that when activated, triggers apoptosis and/or cell death of a therapeutic cell.

In certain embodiments, an artificial cell death polypeptide comprises an inactivated cell surface receptor that comprises an epitope specifically recognized by an antibody, particularly a monoclonal antibody, which is also referred to herein as a monoclonal antibody-specific epitope. When expressed by iPSCs or derivative cells thereof, the inactivated cell surface receptor is signaling inactive or significantly impaired, but can still be specifically recognized by an antibody. The specific binding of the antibody to the inactivated cell surface receptor enables the elimination of the iPSCs or derivative cells thereof by ADCC and/or ADCP mechanisms, as well as, direct killing with antibody drug conjugates with toxins or radionuclides.

In certain embodiments, the inactivated cell surface receptor comprises an epitope that is selected from epitopes specifically recognized by an antibody, including but not limited to, ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, avelumab, ofatumumab, panitumumab, or ustekinumab. In certain embodiments, the inactivated cell surface receptor comprises an epitope that is specifically recognized by cetuximab. In certain embodiments, the inactivated cell surface receptor comprises an epitope that is specifically recognized by trastuzumab. In certain embodiments, the inactivated cell surface receptor comprises an epitope that is specifically recognized by bevacizumab. In certain embodiments, the inactivated cell surface receptor comprises an epitope that is specifically recognized by avelumab. In certain embodiments, the inactivated cell surface receptor comprises an epitope that is specifically recognized by ipilimumab.

Epidermal growth factor receptor, also known as EGFR, ErbB1 and HER1, is a cell-surface receptor for members of the epidermal growth factor family of extracellular ligands. As used herein, “truncated EGFR,” “tEGFR,” “short EGFR” or “sEGFR” refers to an inactive EGFR variant that lacks the EGF-binding domains and the intracellular signaling domains of the EGFR. An exemplary tEGFR variant contains residues 322-333 of domain 2, all of domains 3 and 4 and the transmembrane domain of the native EGFR sequence containing the cetuximab binding epitope. Expression of the tEGFR variant on the cell surface enables cell elimination by an antibody that specifically binds to the tEGFR, such as cetuximab (Erbitux®), as needed. Due to the absence of the EGF-binding domains and intracellular signaling domains, tEGFR is inactive when expressed by iPSCs or derivative cell thereof.

An exemplary inactivated cell surface receptor of the application comprises a tEGFR variant. In certain embodiments, expression of the inactivated cell surface receptor in an engineered immune cell expressing a chimeric antigen receptor (CAR) induces cell suicide of the engineered immune cell when the cell is contacted with an anti-EGFR antibody. Methods of using inactivated cell surface receptors are described in WO2019/070856, WO2019/023396, WO2018/058002, the disclosure of which is incorporated herein by reference. For example, a subject who has previously received an engineered immune cell of the present disclosure that comprises a heterologous polynucleotide encoding an inactivated cell surface receptor comprising a tEGFR variant can be administered an anti-EGFR antibody in an amount effective to ablate in the subject the previously administered engineered immune cell.

In certain embodiments, the anti-EGFR antibody is cetuximab, matuzumab, necitumumab or panitumumab, preferably the anti-EGFR antibody is cetuximab.

In certain embodiments, the tEGFR variant comprises or consists of an amino acid sequence at least 90%, such as at least 90%, 91%, 92%, 93%, 94%, 95%, 96%, 97%, 98%, 99% or 100%, identical to SEQ ID NO: 71, preferably the amino acid sequence of SEQ ID NO: 71.

In some embodiments, the inactivated cell surface receptor comprises one or more epitopes of CD79b, such as an epitope specifically recognized by polatuzumab vedotin. In certain embodiments, the CD79b epitope comprises or consists of an amino acid sequence at least 90%, such as at least 90%, 91%, 92%, 93%, 94%, 95%, 96%, 97%, 98%, 99% or 100%, identical to SEQ ID NO: 78, preferably the amino acid sequence of SEQ ID NO: 78.

In some embodiments, the inactivated cell surface receptor comprises one or more epitopes of CD20, such as an epitope specifically recognized by rituximab. In certain embodiments, the CD20 epitope comprises or consists of an amino acid sequence at least 90%, such as at least 90% 91%, 92%, 93%, 94%, 95%, 96%, 97%, 98%, 99% or 100%, identical to SEQ ID NO: 80, preferably the amino acid sequence of SEQ ID NO: 80.

In some embodiments, the inactivated cell surface receptor comprises one or more epitopes of Her 2 receptor or ErbB, such as an epitope specifically recognized by trastuzumab. In certain embodiments, the monoclonal antibody-specific epitope comprises or consists of an amino acid sequence at least 90%, such as at least 90%, 91%, 92%, 93%, 94%, 95%, 96%, 97%, 98%, 99% or 100%, identical to SEQ ID NO: 82, preferably the amino acid sequence of SEQ ID NO: 82.

### IV. Cytokine Expression

In some embodiments the iPSC cell or a derivative cell thereof optionally comprises an exogenous polynucleotide encoding a cytokine, such as interleukin-15 or interleukin-2.

As used herein “Interleukin-15” or “IL-15” refers to a cytokine that regulates T and NK cell activation and proliferation, or a functional portion thereof. A “functional portion” (“biologically active portion”) of a cytokine refers to a portion of the cytokine that retains one or more functions of full length or mature cytokine. Such functions for IL-15 include the promotion of NK cell survival, regulation of NK cell and T cell activation and proliferation as well as the support of NK cell development from hematopoietic stem cells. As will be appreciated by those of skill in the art, the sequence of a variety of IL-15 molecules are known in the art. In certain embodiments, the IL-15 is a wild-type IL-15. In certain embodiments, the IL-15 is a human IL-15. In certain embodiments, the IL-15 comprises an amino acid sequence at least 90%, such as at least 90%, 91%, 92%, 93%, 94%, 95%, 96%, 97%, 98%, 99% or 100%, identical to SEQ ID NO: 72, preferably the amino acid sequence of SEQ ID NO: 72.

In some embodiments, the IL-15 is a membrane bound form, where all or a functional portion of the IL-15 protein is fused to all or a portion of a transmembrane protein that anchors the expressed IL-15 as a cell membrane-bound polypeptide (mbIL15)”, for example the construct described in U.S. patent U.S. Pat. No. 9,629,877B2, hereby incorporated by reference into the present application.

As used herein “Interleukin-2” refers to a cytokine that regulates T and NK cell activation and proliferation, or a functional portion thereof. In certain embodiments, the IL-2 is a wild-type IL-2. In certain embodiments, the IL-2 is a human IL-2. In certain embodiments, the IL-2 comprises an amino acid sequence at least 90%, such as at least 90%, 91%, 92%, 93%, 94%, 95%, 96%, 97%, 98%, 99% or 100%, identical to SEQ ID NO: 76, preferably the amino acid sequence of SEQ ID NO: 76.

In certain embodiments, an inactivated cell surface receptor comprises a monoclonal antibody-specific epitope operably linked to a cytokine, preferably by an autoprotease peptide sequence. Examples of the autoprotease peptide include, but are not limited to, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In one embodiment, the autoprotease peptide is an autoprotease peptide of porcine teschovirus-1 2A (P2A). In certain embodiments, the autoprotease peptide comprises an amino acid sequence at least 90%, such as at least 90%, 91%, 92%, 93%, 94%, 95%, 96%, 97%, 98%, 99% or 100%, identical to SEQ ID NO: 73, preferably the amino acid sequence of SEQ ID NO: 73.

In certain embodiments, an inactivated cell surface receptor comprises a truncated epithelial growth factor (tEGFR) variant operably linked to an interleukin-15 (IL-15) or IL-2 by an autoprotease peptide sequence. In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 74, preferably the amino acid sequence of SEQ ID NO: 74.

In some embodiments, an inactivated cell surface receptor further comprises a signal sequence. In certain embodiments, the signal sequence comprises an amino acid sequence at least 90%, such as at least 90%, 91%, 92%, 93%, 94%, 95%, 96%, 97%, 98%, 99% or 100%, identical to SEQ ID NO: 77, preferably the amino acid sequence of SEQ ID NO: 77.

In some embodiments, an inactivated cell surface receptor further comprises a hinge domain. In some embodiments, the hinge domain is derived from CD8. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21.

In certain embodiments, an inactivated cell surface receptor further comprises a transmembrane domain. In some embodiments, the transmembrane domain is derived from CD8. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23.

In certain embodiment, an inactivated cell surface receptor comprises one or more epitopes specifically recognized by an antibody in its extracellular domain, a transmembrane region and a cytoplasmic domain. In some embodiments, the inactivated cell surface receptor further comprises a hinge region between the epitope(s) and the transmembrane region. In some embodiments, the inactivated cell surface receptor comprises more than one epitopes specifically recognized by an antibody, the epitopes can have the same or different amino acid sequences, and the epitopes can be linked together via a peptide linker, such as a flexible peptide linker have the sequence of (GGGGS)n, wherein n is an integer of 1-8 (SEQ ID NO: 25). In some embodiments, the inactivated cell surface receptor further comprises a cytokine, such as an IL-15 or IL-2. In certain embodiments, the cytokine is in the cytoplasmic domain of the inactivated cell surface receptor. Preferably, the cytokine is operably linked to the epitope(s) specifically recognized by an antibody, directly or indirectly, via an autoprotease peptide sequence, such as those described herein. In some embodiments, the cytokine is indirectly linked to the epitope(s) by connecting to the transmembrane region via the autoprotease peptide sequence.

Non-limiting exemplary inactivated cell surface receptor regions and sequences are provided in Table 2.

TABLE 2

| | | |
|---|---|---|
| | | SEQ ID |
| Regions | Sequence | NO |

tEGFR-IL15:

| | | |
|---|---|---|
| tEGFR | MRPSGTAGAALLALLAALCPASRAGVRKCKKCEGPCRK | 71 |
| | VCNGIGIGEFKDSLSINATNIKHFKNCTSISGDLHILPVAF | |
| | RGDSFTHTPPLDPQELDILKTVKEITGFLLIQAWPENRTD | |
| | LHAFENLEIIRGRTKQHGQFSLAVVSLNITSLGLRSLKEIS | |
| | DGDVIISGNKNLCYANTINWKKLFGTSGQKTKIISNRGE | |
| | NSCKATGQVCHALCSPEGCWGPEPRDCVSCRNVSRGRE | |
| | CVDKCNLLEGEPREFVENSECIQCHPECLPQAMNITCTG | |
| | RGPDNCIQCAHYIDGPHCVKTCPAGVMGENNTLVWKY | |
| | ADAGHVCHLCHPNCTYGCTGPGLEGCPTNGPKIPSIATG | |
| | MVGALLLLLVVALGIGLFM | |
| P2A | ATNFSLLKQAGDVEENPGP | 73 |
| IL-15 | MRISKPHLRSISIQCYLCLLLNSHFLTEAGIHVFILGCFSA | 72 |
| | GLPKTEANWVNVISDLKKIEDLIQSMHIDATLYTESDVH | |
| | PSCKVTAMKCFLLELQVISLESGDASIHDTVENLIILANN | |
| | SLSSNGNVTESGCKECEELEEKNIKEFLQSFVHIVQMFIN | |
| | TS | |

CD79b-IL15:

| | | |
|---|---|---|
| Signal | MEFGLSWVFLVALFRGVQC | 77 |
| Sequence | | |
| CD79b | ARSEDRYRNPKGSACSRIWQS | 78 |
| epitope | | |
| CD8 (AA | TTTPAPRPPTPAPTIASQPLSLRPEACRPAAGGAVHTRGL | 21 |
| 136-182) | DFACD | |
| CD8 (AA | IYIWAPLAGTCGVLLLSLVIT | 23 |
| 183-203) | | |
| P2A | ATNFSLLKQAGDVEENPGP | 73 |
| IL-15 | MRISKPHLRSISIQCYLCLLLNSHFLTEAGIHVFILGCFSA | 72 |
| | GLPKTEANWVNVISDLKKIEDLIQSMHIDATLYTESDVH | |
| | PSCKVTAMKCFLLELQVISLESGDASIHDTVENLIILANN | |
| | SLSSNGNVTESGCKECEELEEKNIKEFLQSFVHIVQMFIN | |
| | TS | |

CD20 mimitope-IL15:

| | | |
|---|---|---|
| Signal | MEFGLSWVFLVALFRGVQC | 77 |
| Sequence | | |
| CD20 | ACPYANPSLC | 80 |
| mimitope | | |
| Linker | GGGSGGGS | 27 |
| CD8 (AA | TTTPAPRPPTPAPTIASQPLSLRPEACRPAAGGAVHTRGL | 21 |
| 136-182) | DFACD | |
| CD8 (AA | IYIWAPLAGTCGVLLLSLVIT | 23 |
| 183-203) | | |
| P2A | ATNFSLLKQAGDVEENPGP | 73 |
| IL-15 | MRISKPHLRSISIQCYLCLLLNSHFLTEAGIHVFILGCFSA | 72 |
| | GLPKTEANWVNVISDLKKIEDLIQSMHIDATLYTESDVH | |
| | PSCKVTAMKCFLLELQVISLESGDASIHDTVENLIILANN | |
| | SLSSNGNVTESGCKECEELEEKNIKEFLQSFVHIVQMFIN | |
| | TS | |

ErbB epitope-IL15:

| | | |
|---|---|---|
| Signal | MEFGLSWVFLVALFRGVQC | 77 |
| Sequence | | |
| ErbB | EGLACHQLCARGHCWGPGPTQCVNCSQFLRGQECVEE | 82 |
| epitope | CRVLQGLPREYVNARHCLPCHPECQPQNGSVTCFGPEA | |
| | DQCVACAHYKDPPFCVARCPSGVKPDLSYMPIWKFPDE | |
| | EGACQPCPINCTHSCVDLDDKGCPAEQRASPLTSIISAVV | |
| | GILLVVVLGVVFGILIGGGGSGG | |
| P2A | ATNFSLLKQAGDVEENPGP | 73 |
| IL-15 | MRISKPHLRSISIQCYLCLLLNSHFLTEAGIHVFILGCFSA | 72 |
| | GLPKTEANWVNVISDLKKIEDLIQSMHIDATLYTESDVH | |
| | PSCKVTAMKCFLLELQVISLESGDASIHDTVENLIILANN | |
| | SLSSNGNVTESGCKECEELEEKNIKEFLQSFVHIVQMFIN | |
| | TS | |

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 79, preferably the amino acid sequence of SEQ ID NO: 79.

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 81, preferably the amino acid sequence of SEQ ID NO: 81.

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 83, preferably the amino acid sequence of SEQ ID NO: 83.

### V. HLA Expression

In one aspect, MHC I and/or MHC II knock-out and/or knock down can be incorporated in the cells for use in “allogeneic” cell therapies, in which cells are harvested from a subject, modified to knock-out or knock-down, e.g., disrupt, B2M, TAP 1, TAP 2, Tapasin, RFXANK, CIITA, RFX5 and RFXAP gene expression, and then returned to a different subject. Knocking out or knocking down the B2M, TAP 1, TAP 2, Tapasin, RFXANK, CIITA, RFX5 and RFXAP genes as described herein can: (1) prevent Graft versus Host response; (2) prevent Host versus Graft response; and/or (3) improve cell safety and efficacy. Accordingly, in certain embodiments, a presently disclosed invention comprises independently knocking out and/or knocking down one or more genes selected from the group consisting of B2M, TAP 1, TAP 2, Tapasin, RFXANK, CIITA, RFX5 and RFXAP genes in an iPSC cell. In certain embodiments, a presently disclosed method comprises independently knocking out and/or knocking down two genes selected from the group consisting B2M, TAP 1, TAP 2, Tapasin, RFXANK, CIITA, RFX5 and RFXAP genes in an iPSC cell, in particular, B2M and CIITA to achieve class I and II HLA disruption. In certain embodiments, an iPSC or derivative cell thereof of the application can be further modified by introducing an exogenous polynucleotide encoding one or more proteins related to immune evasion, such as non-classical HLA class I proteins (e.g., HLA-E and HLA-G). In particular, disruption of the B2M gene eliminates surface expression of all MHC class I molecules, leaving cells vulnerable to lysis by NK cells through the “missing self” response. Exogenous HLA-E expression can lead to resistance to NK-mediated lysis (Gornalusse et al., Nat Biotechnol. 2017; 35(8): 765-772).

Incorporating MHC I and/or MHC II knock-out and/or knock down in the cells for use in “allogeneic” cell therapies will allow the cell product candidates to escape recognition and destruction by the host immune system. The reduction in allogeneic reactivity enabled by use of this technology will allow repeat dosing of the CAR-modified cell therapies to improve their therapeutic potential. In combination with the extended killing capability of optimized immune cells derived from single genetically engineered cell cloning, the cells will have the capacity for repeat dosing to maximize durability of response and efficacy. Additionally, this technology may permit dosing in patients with limited or no immune preconditioning regimens.

Accordingly, in certain embodiments, an iPSC or derivative cell thereof of the application can be further modified by introducing a third exogenous polynucleotide encoding one or more proteins related to immune evasion, such as non-classical HLA class I proteins (e.g., HLA-E and HLA-G).

In certain embodiments, the iPSC or derivative cell thereof comprises a third exogenous polypeptide encoding at least one of a human leukocyte antigen E (HLA-E) and human leukocyte antigen G (HLA-G). In a particular embodiment, the HLA-E comprises an amino acid sequence at least 90%, such as at least 90%, 91%, 92%, 93%, 94%, 95%, 96%, 97%, 98%, 99% or 100%, identical to SEQ ID NO: 65, preferably the amino acid sequence of SEQ ID NO: 65. In a particular embodiment, the HLA-G comprises an amino acid sequence at least 90%, such as at least 90%, 91%, 92%, 93%, 94%, 95%, 96%, 97%, 98%, 99% or 100%, identical to SEQ ID NO: 68, preferably SEQ ID NO: 68.

In certain embodiments, the third exogenous polynucleotide encodes a polypeptide comprising a signal peptide operably linked to a mature B2M protein that is fused to an HLA-E via a linker. In a particular embodiment, the third exogenous polypeptide comprises an amino acid sequence at least sequence at least 90%, such as at least 90%, 91%, 92%, 93%, 94%, 95%, 96%, 97%, 98%, 99% or 100%, identical to SEQ ID NO: 66.

In other embodiments, the third exogenous polynucleotide encodes a polypeptide comprising a signal peptide operably linked to a mature B2M protein that is fused to an HLA-G via a linker. In a particular embodiment, the third exogenous polypeptide comprises an amino acid sequence at least sequence at least 90%, such as at least 90%, 91%, 92%, 93%, 94%, 95%, 96%, 97%, 98%, 99% or 100%, identical to SEQ ID NO: 69.

### VI. Other Optional Genome Edits

In one embodiment of the above described cell, the genomic editing at one or more selected sites may comprise insertions of one or more exogenous polynucleotides encoding other additional artificial cell death polypeptides, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells thereof. In some embodiments, the exogenous polynucleotides for insertion are operatively linked to (1) one or more exogenous promoters comprising CMV, EF1a, PGK, CAG, UBC, or other constitutive, inducible, temporal-, tissue-, or cell type-specific promoters; or (2) one or more endogenous promoters comprised in the selected sites comprising AAVS1, CLYBL, CCR5, ROSA26, collagen, HTRP, Hll, beta-2 microglobulin, GAPDH, TCR or RUNX1, or other locus meeting the criteria of a genome safe harbor. In some embodiments, the genome-engineered iPSCs generated using the above method comprise one or more different exogenous polynucleotides encoding proteins comprising caspase, thymidine kinase, cytosine deaminase, B-cell CD20, ErbB2 or CD79b wherein when the genome-engineered iPSCs comprise two or more suicide genes, the suicide genes are integrated in different safe harbor locus comprising AAVS1, CCR5, ROSA26, collagen, HTRP, Hll, Hll, beta-2 microglobulin, GAPDH, TCR or RUNX1. Other exogenous polynucleotides encoding proteins may include those encoding PET reporters, homeostatic cytokines, and inhibitory checkpoint inhibitory proteins such as PD1, PD-L1, and CTLA4 as well as proteins that target the CD47/signal regulatory protein alpha (SIRPα) axis.

In one aspect, the cell may comprise an exogenous polynucleotide encoding a CD16 protein and/or an NKG2D protein, wherein the CD16 protein and the NKG2D protein may be operably linked by an autoprotease peptide as disclosed in co-pending patent application PCT/US23/68079. Accordingly, in some aspects, cells of the present invention may comprise genetically engineered iPSCs and cells derived therefrom that exogenously express recombinant CD16 and recombinant NKG2D. The surface receptor CD16 (FcγRIIIA) affects human natural killer (NK) cells during maturation. NK cells bind the Fc portion of IgG via CD16, and execute antibody-dependent cellular cytotoxicity, which is critical for the effectiveness of several anti-tumor monoclonal antibody therapies. NKG2D is an stimulatory/activating receptor that is mostly expressed on cells of the cytotoxic arm of the immune system including NK cells and subsets of T cells. NKG2D is crucial in diverse aspects of innate and adaptive immune functions. In some embodiments, CD16 and NKG2D are expressed from in a single polynucleotide construct as it is advantageous to reduce the number of gene edits of a cell.

In some embodiments, the polynucleotide construct encoding the CD16 protein and the NKG2D protein also includes a polynucleotide sequence encoding an autoprotease peptide or self-cleaving peptide. In some embodiments, an exogenous polynucleotide construct encoding the CD16 protein, the NKG2D protein and the self-cleaving peptide is introduced into the iPSC or derivative cell thereof. The exogenous or isolated polynucleotide construct can be introduced into a gene locus of the iPSC or derivative cell thereof.

In some embodiments, the exogenous polynucleotide construct comprises the nucleic acid sequence of SEQ ID NO: 185. In some embodiments, the exogenous polynucleotide construct encodes for the amino acid sequence of SEQ ID NO: 186.

In some embodiments, the CD16 protein (which is also referred to as “low affinity immunoglobulin gamma Fc region receptor III-A” or “Fc gamma receptor IIIa”) is a wildtype CD16 protein. In some embodiments, the human wildtype CD16 protein has the amino acid sequence set forth in NCBI Ref. Seq. No. NP_000560.7 or UniProt No. P08637. In some instance, the coding sequence of human wildtype CD16 is set forth in NCBI Ref. No. NM_000569.8.

In some embodiments, the CD16 protein is a CD16 variant protein. In some instances, the CD16 variant protein has an amino acid sequence having at least 90%, e.g., at least 90%, at least 91%, at least 92%, at least 93%, at least 94%, at least 95%, at least 96%, at least 97%, at least 98%, or at least 99% sequence identity to wildtype CD16 such as that of SEQ ID NO: 187. In some instances, the CD16 variant is a high affinity CD16 variant. In other instances, the CD16 variant is a non-cleavable CD16 variant. In some instances, the CD16 variant is a high affinity and non-cleavable CD16 variant.

In some embodiments, the CD16 variant comprises one or more amino acid substitutions selected from the group consisting of F158V, F176V, S197P, D205A, S219A, T220A, and any combination thereof. In some embodiments, the CD16 variant has an F158V substitution and one or more substitutions selected from F176V, S197P, D205A, S219A, T220A, and any combination thereof. In one embodiment, the CD16 variant has an F176V substitution and one or more substitutions selected from F158V, S197P, D205A, S219A, T220A, and any combination thereof. In many embodiments, the CD16 variant has an S197P, substitution and one or more substitutions selected from F158V, F176V, D205A, S219A, T220A, and any combination thereof. In various embodiments, the CD16 variant has a D205A substitution and one or more substitutions selected from F158V, F176V, S197P, S219A, T220A, and any combination thereof. In some embodiments, the CD16 variant has a substitution and one or more substitutions selected from F158V, F176V, S197P, D205A, S219A, T220A, and any combination thereof. In some embodiments, the CD16 variant has an S219A substitution and one or more substitutions selected from F158V, F176V, S197P, D205A, T220A, and any combination thereof. In some embodiments, the CD16 variant has a T220A substitution and one or more substitutions selected from F158V, F176V, S197P, D205A, S219A, T220A, and any combination thereof. In some embodiments, the variant CD16 protein has the sequence of SEQ ID NO: 188. In some embodiments, the nucleic acid sequence encoding the variant CD16 protein has the sequence of SEQ ID NO: 189. In some embodiments, the wildtype CD16 protein has the sequence of SEQ ID NO: 187.

In some embodiments, the NKG2D protein (which is also referred to as NKG2-D type II integral membrane protein, CD314, killer cell lectin-like receptor subfamily KI member 1 or KLRK1) is a wildtype NKG2D protein. In some embodiments, the human wildtype NKG2D protein has the amino acid sequence set forth in NCBI Ref. Seq. Nos. NP_001186734.1 or NP_031386.2 or UniProt No. P26718. In some instance, the coding sequence of human wildtype NKG2D is set forth in NCBI Ref Nos. NM_001199805.1 or NM_007360.3. In some embodiments, the NKG2D protein is a NKG2D variant protein. In some instances, the NKG2D variant protein has an amino acid sequence having at least 90%, e.g., at least 90%, at least 91%, at least 92%, at least 93%, at least 94%, at least 95%, at least 96%, at least 97%, at least 98%, or at least 99% sequence identity to wildtype NKG2D such as that of SEQ ID NO: 190. In some embodiments, the NKG2D protein has the amino acid sequence of SEQ ID NO: 190. In some embodiments, the nucleic acid sequence encoding the NKG2D protein has sequence of SEQ ID NO: 191.

As discussed above, provided herein are constructs containing autoprotease peptide sequences including 2A peptides that can induce ribosomal skipping during translation of an polypeptide. 2A peptides function to “cleave” an mRNA transcript by making the ribosome skip the synthesis of a peptide bond at the C-terminus, between the glycine (G) and proline (P) residues, thereby leading to separation between the end of the 2A sequence and the next peptide downstream. 2A peptides include, but are not limited to, a porcine teschovirus-1 2A (P2A) peptide, a foot-and-mouth disease virus (FMDV) 2A (F2A) peptide, an Equine Rhinitis A Virus (ERAV) 2A (E2A) peptide, a Thosea asigna virus 2A (T2A) peptide, a cytoplasmic polyhedrosis virus 2A (BmCPV2A) peptide, and a Flacherie Virus 2A (BmIFV2A) peptide.

An exemplary P2A peptide can include an amino acid sequence having at least 90%, such as 90%, 91%, 92%, 93%, 94%, 95%, 96%, 97%, 98%, 99% or 100% sequence identity to SEQ ID NO: 192. In some embodiment, the P2A peptide has the amino acid sequence of SEQ ID NO: 192.

Another optional genome edit is the insertion of a polynucleotide encoding a membrane-bound interleukin 12 (IL-12) comprising a first polypeptide comprising an IL-12 alpha subunit p35, a second polypeptide comprising an IL-12 beta subunit p40 and a transmembrane fused to the terminus of the first and/or second IL-12 subunit polypeptide as disclosed in co-pending patent application PCT/US23/68105. In certain embodiments, the polynucleotide encoding the membrane bound IL-12 is fused to a polynucleotide encoding an ADAM17 protease cleavage site peptide for the activation induced release of the IL-12 through the protease ADAM17. ADAM17 is expressed by activated lymphocytes and is directly involved in the liberation of other immune mediators like TNFa that are similarly presented as a membrane anchored form. When this membrane tethered IL-12 is expressed on engineered iNK or T cells, it remains cell associated. Upon cell activation and the increased expression of ADAM17, the protease cleaves the membrane stalk and releases IL-12 into the extracellular space. This type of regulation ensures that the activities of the IL-12 are confined to spaces surrounding the tumor where the engineered immune cells engage their targets on the tumor cells that cause their activation. Accordingly, the cell of the invention may further comprise (i) an exogenous polynucleotide encoding a membrane-bound interleukin 12 (IL-12) comprising a first polypeptide comprising an IL-12 alpha subunit p35 or a polypeptide at least 90% similar thereto, a second polypeptide comprising an IL-12 beta subunit p40 or a polypeptide at least 90% similar thereto, and a transmembrane domain fused to the terminus of the first and/or second IL-12 subunit polypeptide.

In some other embodiments, the genome-engineered iPSCs generated using the method provided herein comprise in/del at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the iPSCs or derivative cells thereof.

### VII. Targeted Genome Editing at Selected Locus in iPSCs

According to embodiments of the application, one or more of the exogenous polynucleotides are integrated at one or more loci on the chromosome of an iPSC.

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted or disrupted at the insertion site during targeted editing, an endogenous gene comprising the affected sequence can be knocked-out or knocked-down due to the sequence deletion or disruption. Therefore, targeted editing can also be used to disrupt endogenous gene expression with precision. Similarly used herein is the term “targeted integration,” referring to a process involving insertion of one or more exogenous sequences at pre-selected sites in the genome, with or without deletion of an endogenous sequence at the insertion site.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

Available endonucleases capable of introducing specific and targeted DSBs include, but not limited to, zinc-finger nucleases (ZFN), transcription activator-like effector nucleases (TALEN), RNA-guided CRISPR (Clustered Regular Interspaced Short Palindromic Repeats) systems. Additionally, DICE (dual integrase cassette exchange) system utilizing phiC31 and Bxbl integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the Fokl nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in U.S. Patent Application No. 2011/0145940, which is herein incorporated by reference. The most recognized example of a TALEN in the art is a fusion polypeptide of the Fokl nuclease to a TAL effector DNA binding domain.

Another example of a targeted nuclease that finds use in the subject methods is a targeted Spoll nuclease, a polypeptide comprising a Spol 1 polypeptide having nuclease activity fused to a DNA binding domain, e.g. a zinc finger DNA binding domain, a TAL effector DNA binding domain, etc. that has specificity for a DNA sequence of interest. See, for example, U.S. Application No. 61/555,857, the disclosure of which is incorporated herein by reference.

Additional examples of targeted nucleases suitable for the present application include, but not limited to Bxbl, phiC3 1, R4, PhiBTI, and Wp/SPBc/TP901-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr, restriction endonucleases; meganucleases; homing endonucleases, and the like. As an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. As another example, CRISPR/Cpf1 comprises two major components: (1) a CPf1 endonuclease and (2) a crRNA. When co-expressed, the two components form a ribonucleoprotein (RNP) complex that is recruited to a target DNA sequence comprising PAM and a seeding region near PAM. The crRNA can be combined to form a chimeric guide RNA (gRNA) to guide Cpf1 to target selected sequences. These two components can then be delivered to mammalian cells via transfection or transduction.

MAD7 is an engineered Cas12a variant originating from the bacterium Eubacterium rectale that has a preference for 5′-TTTN-3′ and 5′-CTTN-3′ PAM sites and does not require a tracrRNA. See, for example, PCT Publication No. 2018/236548, the disclosure of which is incorporated herein by reference.

DICE mediated insertion uses a pair of recombinases, for example, phiC31 and Bxbl, to provide unidirectional integration of an exogenous DNA that is tightly restricted to each enzymes' own small attB and attP recognition sites. Because these target att sites are not naturally present in mammalian genomes, they must be first introduced into the genome, at the desired integration site. See, for example, U.S. Application Publication No. 2015/0140665, the disclosure of which is incorporated herein by reference.

One aspect of the present application provides a construct comprising one or more exogenous polynucleotides for targeted genome integration. In one embodiment, the construct further comprises a pair of homologous arm specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cpf1 expression cassette, and a gRNA comprising a guide sequence specific to a desired integration site to the cell to enable a Cpf1-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Sites for targeted integration include, but are not limited to, genomic safe harbors, which are intragenic or extragenic regions of the human genome that, theoretically, are able to accommodate predictable expression of newly integrated DNA without adverse effects on the host cell or organism. In certain embodiments, the genome safe harbor for the targeted integration is one or more loci of genes selected from the group consisting of AAVS1, CLYBL, CCR5, ROSA26, collagen, HTRP, Hll, GAPDH, TCR and RUNX1 genes.

In other embodiments, the site for targeted integration is selected for deletion or reduced expression of an endogenous gene at the insertion site. As used herein, the term “deletion” with respect to expression of a gene refers to any genetic modification that abolishes the expression of the gene. Examples of “deletion” of expression of a gene include, e.g., a removal or deletion of a DNA sequence of the gene, an insertion of an exogenous polynucleotide sequence at a locus of the gene, and one or more substitutions within the gene, which abolishes the expression of the gene.

Genes for target deletion include, but are not limited to, genes of major histocompatibility complex (MHC) class I and MHC class II proteins. Multiple MHC class I and class II proteins must be matched for histocompatibility in allogeneic recipients to avoid allogeneic rejection problems. “MHC deficient”, including MHC-class I deficient, or MHC-class II deficient, or both, refers to cells that either lack, or no longer maintain, or have reduced level of surface expression of a complete MHC complex comprising a MHC class I protein heterodimer and/or a MHC class II heterodimer, such that the diminished or reduced level is less than the level naturally detectable by other cells or by synthetic methods. MHC class I deficiency can be achieved by functional deletion of any region of the MHC class I locus (chromosome 6p21), or deletion or reducing the expression level of one or more MHC class-I associated genes including, not being limited to, beta-2 microglobulin (B2M) gene, TAP 1 gene, TAP 2 gene and Tapasin genes. For example, the B2M gene encodes a common subunit essential for cell surface expression of all MHC class I heterodimers. B2M null cells are MHC-I deficient. MHC class II deficiency can be achieved by functional deletion or reduction of MHC-II associated genes including, not being limited to, RFXANK, CIITA, RFX5 and RFXAP. CIITA is a transcriptional coactivator, functioning through activation of the transcription factor RFX5 required for class II protein expression. CIITA null cells are MHC-II deficient. In certain embodiments, one or more of the exogenous polynucleotides are integrated at one or more loci of genes selected from the group consisting of B2M, TAP 1, TAP 2, Tapasin, RFXANK, CIITA, RFX5 and RFXAP genes to thereby delete or reduce the expression of the gene(s) with the integration. Other genes that may be targeted for deletion include NKG2A, CD38, CD70 and CD33.

In certain embodiments, the exogenous polynucleotides are integrated at one or more loci on the chromosome of the cell, preferably the one or more loci are of genes selected from the group consisting of AAVS1, CLYBL, CCR5, ROSA26, collagen, HTRP, Hl 1, GAPDH, RUNX1, B2M, TAPI, TAP2, Tapasin, NLRC5, CIITA, RFXANK, CIITA, RFX5, RFXAP, TCR a or b constant region, NKG2A, NKG2D, CD33, CD38, CIS, CBL-B, SOCS2, PD1, CTLA4, LAG3, TIM3, or TIGIT genes, provided at least one of the one or more loci is of a MHC gene, such as a gene selected from the group consisting of B2M, TAP 1, TAP 2, Tapasin, RFXANK, CIITA, RFX5 and RFXAP genes. Preferably, the one or more exogenous polynucleotides are integrated at a locus of an MHC class-I associated gene, such as a beta-2 microglobulin (B2M) gene, TAP 1 gene, TAP 2 gene or Tapasin gene; and at a locus of an MHC-II associated gene, such as a RFXANK, CIITA, RFX5, RFXAP, or CIITA gene; and optionally further at a locus of a safe harbor gene selected from the group consisting of AAVS1, CLYBL, CCR5, ROSA26, collagen, HTRP, Hll, GAPDH, TCR and RUNX1 genes. More preferably, the one or more of the exogenous polynucleotides are integrated at the loci of CIITA, AAVS1 and B2M genes.

In certain embodiments, (i) the first exogenous polynucleotide is integrated at a locus of AAVS1 gene or CLYBL gene; (ii) the second exogenous polypeptide is integrated at a locus of CIITA gene; and (iii) the third exogenous polypeptide is integrated at a locus of B2M gene; wherein integrations of the exogenous polynucleotides delete or reduce expression of CIITA and B2M genes.

In certain embodiments, (i) the first exogenous polynucleotide comprises the polynucleotide sequence having at least 90%, 91%, 92%, 93%, 94%, 95%, 96%, 97%, 98%, 99% or 100% sequence identity to one or more sequences selected from the group consisting of SEQ ID NOs: 131-156, and 171-184; (ii) the second exogenous polynucleotide comprises the polynucleotide sequence having at least 90%, 91%, 92%, 93%, 94%, 95%, 96%, 97%, 98%, 99% or 100% sequence identity to SEQ ID NO: 75; and (iii) the third exogenous polynucleotide comprises the polynucleotide sequence having at least 90%, 91%, 92%, 93%, 94%, 95%, 96%, 97%, 98%, 99% or 100% sequence identity to SEQ ID NO: 67.

In certain embodiments, (i) the first exogenous polynucleotide comprises the polynucleotide sequence of one or more sequences selected from the group consisting of SEQ ID NOs: 131-156, and 171-184; (ii) the second exogenous polynucleotide comprises the polynucleotide sequence of SEQ ID NO: 75; and (iii) the third exogenous polynucleotide comprises the polynucleotide sequence of SEQ ID NO: 67.

### VIII. Derivative Cells

In another aspect, the invention relates to a cell derived from differentiation of an iPSC, a derivative cell. As described above, the genomic edits introduced into the iPSC are retained in the derivative cell. In certain embodiments of the derivative cell obtained from iPSC differentiation, the derivative cell is a hematopoietic cell, including, but not limited to, HSCs (hematopoietic stem and progenitor cells), hematopoietic multipotent progenitor cells, T cell progenitors, NK cell progenitors, T cells, NKT cells, NK cells, B cells, antigen presenting cells (APC), monocytes and macrophages. In certain embodiments, the derivative cell is an immune effector cell, such as a NK cell or a T cell.

In certain embodiments, the application provides a natural killer (NK) cell or a T cell comprising: (i) a first exogenous polynucleotide encoding a chimeric antigen receptor (CAR); (ii) a second exogenous polynucleotide encoding a truncated epithelial growth factor (tEGFR) variant and an interleukin 15 (IL-15), wherein the tEGFR variant and ILL-15 are operably linked by an autoprotease peptide sequence, such as autoprotease peptide sequence of porcine teschovirus-1 2A (P2A); and (iii) a deletion or reduced expression of an MHC class I associated gene and an MHC class II associated gene, such as an MHC class-I associated gene selected from the group consisting of a B2M gene, TAP 1 gene, TAP 2 gene and Tapasin gene, and an MHC-II associated gene selected from the group consisting of a RFXANK gene, CIITA gene, RFX5 gene, RFXAP gene, and CIITA gene, preferably the B2M gene and CIITA gene.

In certain embodiments, the NK cell or T cell further comprises a third exogenous polynucleotide encoding at least one of a human leukocyte antigen E (HLA-E) and a human leukocyte antigen G (HLA-G).

Also provided is a NK cell or a T cell comprising: (i) a first exogenous polynucleotide encoding a chimeric antigen receptor (CAR) having the amino acid sequence of one or more selected from the group consisting of SEQ ID NOs: 157-170; (ii) a second exogenous polynucleotide encoding a truncated epithelial growth factor (tEGFR) variant having the amino acid sequence of SEQ ID NO: 71, an autoprotease peptide having the amino acid sequence of SEQ ID NO: 73, and interleukin 15 (IL-15) having the amino acid sequence of SEQ ID NO: 72; and (iii) a third exogenous polynucleotide encoding a human leukocyte antigen E (HLA-E) having the amino acid sequence of SEQ ID NO: 66;
wherein the first, second and third exogenous polynucleotides are integrated at loci of AAVS1, CIITA and B2M genes, respectively, to thereby delete or reduce expression of CIITA and B2M.

In certain embodiments, the first exogenous polynucleotide comprises the polynucleotide sequence of one or more selected from the group consisting of SEQ ID NOs: 131-156, and 171-184; the second exogenous polynucleotide comprises the polynucleotide sequence of SEQ ID NO: 75; and the third exogenous polynucleotide comprises the polynucleotide sequence of SEQ ID NO: 67.

Also provided is a CD34+ hematopoietic progenitor cell (IPC) derived from an induced pluripotent stem cell (iPSC) comprising: (i) a first exogenous polynucleotide encoding a chimeric antigen receptor (CAR); (ii) a second exogenous polynucleotide encoding an inactivated cell surface receptor that comprises a monoclonal antibody-specific epitope and an interleukin 15 (IL-15), wherein the inactivated cell surface receptor and IL-15 are operably linked by an autoprotease peptide sequence; and (iii) a deletion or reduced expression of one or more of B2M, TAP 1, TAP 2, Tapasin, RFXANK, CIITA, RFX5 and RFXAP genes.

In certain embodiments, the CD34+ HPC further comprises a third exogenous polynucleotide encoding a human leukocyte antigen E (HLA-E) and/or human leukocyte antigen G (HLA-G).

In certain embodiments, the CAR comprises (i) a signal peptide; (ii) an extracellular domain comprising a binding domain that specifically binds to Nectin4; (iii) a hinge region; (iv) a transmembrane domain; (v) an intracellular signaling domain; and (vi) a co-stimulatory domain, such as a co-stimulatory domain comprising a CD28 signaling domain.

Also provided is a method of manufacturing the derivative cell. The method comprises differentiating the iPSC under conditions for cell differentiation to thereby obtain the derivative cell.

An iPSC of the application can be differentiated by any method known in the art. Exemplary methods are described in U.S. Pat. Nos. 8,846,395, 8,945,922, 8,318,491, WO2010/099539, WO2012/109208, WO2017/070333, WO2017/179720, WO2016/010148, WO2018/048828, WO2019/157597, WO2022/120334, WO2022/133169, WO2022/216624, WO2022/216514, and WO2022/216524, each of which are herein incorporated by reference in its entirety. The differentiation protocol may use feeder cells or may be feeder-free. As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type.

In another embodiment of the invention, the iPSC derivative cells of the invention are NK cells which are prepared by a method of differentiating an iPSC into an NK cell by subjecting the cells to a differentiation protocol including the addition of recombinant human IL-12p70 for the final 24 hours of culture. By including the IL-12 in the differentiation protocol, cells that are primed with IL-12 demonstrate more rapid cell killing compared to those that are differentiated in the absence of IL-12. In addition, the cells differentiated using the IL-12 conditions demonstrate improved cancer cell growth inhibition.

### IX. Polynucleotides, Vectors, and Host Cells

### (1) Nucleic Acids Encoding a CAR

In another general aspect, the invention relates to an isolated nucleic acid encoding a chimeric antigen receptor (CAR) useful for an invention according to embodiments of the application. It will be appreciated by those skilled in the art that the coding sequence of a CAR can be changed (e.g., replaced, deleted, inserted, etc.) without changing the amino acid sequence of the protein. Accordingly, it will be understood by those skilled in the art that nucleic acid sequences encoding CARs of the application can be altered without changing the amino acid sequences of the proteins.

In certain embodiments, the isolated nucleic acid encodes a CAR targeting Nectin4. In a particular embodiment, the isolated nucleic acid encoding the CAR comprises a polynucleotide sequence at least 10%, at least 20%, at least 30%, at least 40%, at least 50%, at least 60%, at least 70%, at least 80%, or at least 90%, such as at least 90%, 91%, 92%, 93%, 94%, 95%, 96%, 97%, 98% or 100%, identical to one or more sequences selected from SEQ ID NOs: 131-156, and 171-184.

In another general aspect, the application provides a vector comprising a polynucleotide sequence encoding a CAR useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a CAR in the cell. Conventional cloning techniques or artificial gene synthesis can be used to generate a recombinant expression vector according to embodiments of the application.

In a particular aspect, the application provides vectors for targeted integration of a CAR useful for an invention according to embodiments of the application. In certain embodiments, the vector comprises an exogenous polynucleotide having, in the 5′ to 3′ order, (a) a promoter; (b) a polynucleotide sequence encoding a CAR according to an embodiment of the application; and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EF1a, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to, BGH, hGH, and PGK.

In certain embodiments, the polynucleotide sequence encoding a CAR comprises the polynucleotide sequence at least 90%, such as at least 90%, 91%, 92%, 93%, 94%, 95%, 96%, 97%, 98% or 100%, identical to one or more selected from the group consisting of SEQ ID NOs: 131-156, and 171-184.

In some embodiments, the vector further comprises a left homology arm and a right homology arm flanking the exogenous polynucleotide. As used herein, “left homology arm” and “right homology arm” refers to a pair of nucleic acid sequences that flank an exogenous polynucleotide and facilitate the integration of the exogenous polynucleotide into a specified chromosomal locus. Sequences of the left and right arm homology arms can be designed based on the integration site of interest. In some embodiments, the left or right arm homology arm is homologous to the left or right side sequence of the integration site.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 84, 87, 90, 193, 195, 197, 199, 201, 203, 205, 207, 209, 211, 213, or 215. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 85, 88, 91, 194, 196, 198, 200, 202, 204, 206, 208, 210, 212, 214, or 216.

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 92, preferably the polynucleotide sequence of SEQ ID NO: 92. Table 3 provides a list of exemplary homology arm sequences and corresponding guide sequences for facilitating integration of an exogenous polynucleotide at various loci.

TABLE 3

| | | | | guide |
|---|---|---|---|---|
| Locus | gRNA | LHA | RHA | sequence |
| AAVS1 | AAS1 | ACTCTGCCCCAGG | CCACCCCACAGTGG | TCTGTC |
| (PPPIR | gRNA2 | CCTCCTTACCATTC | GGCCACTAGGGACA | CCCTCC |
| 12C) | intron 1 | CCCTTCGACCTAC | GGATTGGTGACAGA | ACCCC |
| | | TCTCTTCCGCATTG | AAAGCCCCATCCTT | ACA |
| | | GAGTCGCTTTAAC | AGGCCTCCTCCTTCC | (SEQ ID |
| | | TGGCCCTGGCTTT | TAGTCTCCTGATATT | NO: 217) |
| | | GGCAGCCTGTGCT | GGGTCTAACCCCCA | |
| | | GACCCATGCAGTC | CCTCCTGTTAGGCA | |
| | | CTCCTTACCATCCC | GATTCCTTATCTGGT | |
| | | TCCCTCGACTTCCC | GACACACCCCCATT | |
| | | CTCTTCCGATGTTG | TCCTGGAGCCATCT | |
| | | AGCCCCTCCAGCC | CTCTCCTTGCCAGA | |
| | | GGTCCTGGACTTT | ACCTCTAAGGTTTG | |
| | | GTCTCCTTCCCTGC | CTTACGATGGAGCC | |
| | | CCTGCCCTCTCCTG | AGAGAGGATCCTGG | |
| | | AACCTGAGCCAGC | GAGGGAGAGCTTGG | |
| | | TCCCATAGCTCAG | CAGGGGGTGGGAGG | |
| | | TCTGGTCTATCTGC | GAAGGGGGGGATGC | |
| | | CTGGCCCTGGCCA | GTGACCTGCCCGGT | |
| | | TTGTCACTTTGCGC | TCTCAGTGGCCACC | |
| | | TGCCCTCCTCTCGC | CTGCGCTACCCTCTC | |
| | | CCCCGAGTGCCCT | CCAGAACCTGAGCT | |
| | | TGCTGTGCCGCCG | GCTCTGACGCGGCC | |
| | | GAACTCTGCCCTC | GTCTGGTGCGTTTC | |
| | | TAACGCTGCCGTC | ACTGATCCTGGTGC | |
| | | TCTCTCCTGAGTCC | TGCAGCTTCCTTAC | |
| | | GGACCACTTTGAG | ACTTCCCAAGAGGA | |
| | | CTCTACTGGCTTCT | GAAGCAGTTTGGAA | |
| | | GCGCCGCCTCTGG | AAACAAAATCAGAA | |
| | | CCCACTGTTTCCCC | TAAGTTGGTCCTGA | |
| | | TTCCCAGGCAGGT | GTTCTAACTTTGGCT | |
| | | CCTGCTTTCTCTGA | CTTCACCTTTCTAGT | |
| | | CCTGCATTCTCTCC | CCCCAATTTATATTG | |
| | | CCTGGGCCTGTGC | TTCCTCCGTGCGTCA | |
| | | CGCTTTCTGTCTGC | GTTTTACCTGTGAG | |
| | | AGCTTGTGGCCTG | ATAAGGCCAGTAGC | |
| | | GGTCACCTCTACG | CAGCCCCGTCCTGG | |
| | | GCTGGCCCAGATC | CAGGGCTGTGGTGA | |
| | | CTTCCCTGCCGCCT | GGAGGGGGGTGTCC | |
| | | CCTTCAGGTTCCG | GTGTGGAAAACTCC | |
| | | TCTTCCTCCACTCC | CTTTGTGAGAATGG | |
| | | CTCTTCCCCTTGCT | TGCGTCCTAGGTGT | |
| | | CTCTGCTGTGTTGC | TCACCAGGTCGTGG | |
| | | TGCCCAAGGATGC | CCGCCTCTACTCCCT | |
| | | TCTTTCCGGAGCA | TTCTCTTTCTCCATC | |
| | | CTTCCTTCTCGGCG | CTTCTTTCCTTAAAG | |
| | | CTGCACCACGTGA | AGTCCCCAGTGCTA | |
| | | TGTCCTCTGAGCG | TCTGGGACATATTC | |
| | | GATCCTCCCCGTG | CTCCGCCCAGAGCA | |
| | | TCTGGGTCCTCTCC | GGGTCCCGCTTCCC | |
| | | GGGCATCTCTCCT | TAAGGCCCTGCTCT | |
| | | CCCTCACCCAACC | GGGCTTCTGGGTTT | |
| | | CCATGCCGTCTTC | GAGTCCTTGGCAAG | |
| | | ACTCGCTGGGTTC | CCCAGGAGAGGCGC | |
| | | CCTTTTCCTTCTCC | TCAGGCTTCCCTGTC | |
| | | TTCTGGGGCCTGT | CCCCTTCCTCGTCCA | |
| | | GCCATCTCTCGTTT | CCATCTCATGCCCCT | |
| | | CTTAGGATGGCCT | GGCTCTCCTGCCCCT | |
| | | TCTCCGACGGATG | TCCCTACAGGGGTT | |
| | | TCTCCCTTGCGTCC | CCTGGCTCTGCTCTT | |
| | | CGCCTCCCCTTCTT | CAGACTGAGCCCCG | |
| | | GTAGGCCTGCATC | TTCCCCTGCATCCCC | |
| | | ATCACCGTTTTTCT | GTACCCCTGCATCC | |
| | | GGACAACCCCAAA | CCCTTCCCCTGCATC | |
| | | GTACCCCGTCTCC | CCCCAGAGGCCCCA | |
| | | CTGGCTTTAGCCA | GGCCACCTACTTGG | |
| | | CCTCTCCATCCTCT | CCTGGACCCCACGA | |
| | | TGCTTTCTTTGCCT | GAGGCCACCCCAGC | |
| | | GGACACCCCGTTC | CCTGTCTACCAGGC | |
| | | TCCTGTGGATTCG | TGCCTTTTGGGTGG | |
| | | GGTCACCTCTCAC | ATTCTCCTCCAACTG | |
| | | TCCTTTCATTTGGG | TGGGGTGACTGCTT | |
| | | CAGCTCCCCTACC | GGCAAACTCACTCT | |
| | | CCCCTTACCTCTCT | TCGGGGTATCCCAG | |
| | | AGTCTGTGCTAGC | GAGGCCTGGAGCAT | |
| | | TCTTCCAGCCCCCT | TGGGGTGGGCTGGG | |
| | | GTCATGGCATCTT | GTTCAGAGAGGAGG | |
| | | CCAGGGGTCCGAG | GATTCCCTTCTCAG | |
| | | AGCTCAGCTAGTC | GTTACGTGGCCAAG | |
| | | TTCTTCCTCCAACC | AAGCAGGGGAGCTG | |
| | | CGGGCCCCTATGT | GGTTTGGGTCAGGT | |
| | | CCACTTCAGGACA | CTGGGTGTGGGGTG | |
| | | GCATGTTTGCTGC | ACCAGCTTATGCTG | |
| | | CTCCAGGGATCCT | TTTGCCCAGGACAG | |
| | | GTGTCCCCGAGCT | CCTAG | |
| | | GGGACCACCTTAT | (SEQ ID NO: 194) | |
| | | ATTCCCAGGGCCG | | |
| | | GTTAATGTGGCTC | | |
| | | TGGTTCTGGGTAC | | |
| | | TTTTATCTGTCCCC | | |
| | | T | | |
| | | (SEQ ID NO: 193) | | |
| B2M | B2M gRNA2 | GCATATAAAACCT | ATCCAGCAGAGAAT | CATTCT |
| | or | CAGCAGAAATAAA | GGAAAGTCAAATTT | CTGCT |
| | 4 exon 2 | GAGGTTTTGTTGTT | CCTGAATTGCTATG | GGATG |
| | | TGGTAAGAACATA | TGTCTGGGTTTCATC | ACGT |
| | | CCTTGGGTTGGTT | CATCCGACATTGAA | (SEQ ID |
| | | GGGCACGGTGGCT | GTTGACTTACTGAA | NO: 218) |
| | | CGTGCCTGTAATC | GAATGGAGAGAGA | CTCAC |
| | | CCAACACTTTGGG | ATTGAAAAAGTGGA | GTCAT |
| | | AGGCCAAGGCAGG | GCATTCAGACTTGT | CCAGC |
| | | CTGATCACTTGAA | CTTTCAGCAAGGAC | AGAGA |
| | | GTTGGGAGTTCAA | TGGTCTTTCTATCTC | (SEQ ID |
| | | GACCAGCCTGGCC | TTGTACTACACTGA | NO: 240) |
| | | AACATGGTGAAAT | ATTCACCCCCACTG | |
| | | CCCGTCTCTACTG | AAAAAGATGAGTAT | |
| | | AAAATACAAAAAT | GCCTGCCGTGTGAA | |
| | | TAACCAGGCATGG | CCATGTGACTTTGTC | |
| | | TGGTGTGTGCCTG | ACAGCCCAAGATAG | |
| | | TAGTCCCAGGAAT | TTAAGTGGGGTAAG | |
| | | CACTTGAACCCAG | TCTTACATTCTTTTG | |
| | | GAGGCGGAGGTTG | TAAGCTGCTGAAAG | |
| | | CAGTGAGCTGAGA | TTGTGTATGAGTAG | |
| | | TCTCACCACTGCA | TCATATCATAAAGC | |
| | | CACTGCACTCCAG | TGCTTTGATATAAA | |
| | | CCTGGGCAATGGA | AAAGGTCTATGGCC | |
| | | ATGAGATTCCATC | ATACTACCCTGAAT | |
| | | CCAAAAAATAAAA | GAGTCCCATCCCAT | |
| | | AAATAAAAAAATA | CTGATATAAACAAT | |
| | | AAGAACATACCTT | CTGCATATTGGGAT | |
| | | GGGTTGATCCACT | TGTCAGGGAATGTT | |
| | | TAGGAACCTCAGA | CTTAAAGATCAGAT | |
| | | TAATAACATCTGC | TAGTGGCACCTGCT | |
| | | CACGTATAGAGCA | GAGATACTGATGCA | |
| | | ATTGCTATGTCCC | CAGCATGGTTTCTG | |
| | | AGGCACTCTACTA | AACCAGTAGTTTCC | |
| | | GACACTTCATACA | CTGCAGTTGAGCAG | |
| | | GTTTAGAAAATCA | GGAGCAGCAGCAGC | |
| | | GATGGGTGTAGAT | ACTTGCACAAATAC | |
| | | CAAGGCAGGAGCA | ATATACACTCTTAA | |
| | | GGAACCAAAAAG | CACTTCTTACCTACT | |
| | | AAAGGCATAAACA | GGCTTCCTCTAGCTT | |
| | | TAAGAAAAAAAAT | TTGTGGCAGCTTCA | |
| | | GGAAGGGGTGGA | GGTATATTTAGCAC | |
| | | AACAGAGTACAAT | TGAACGAACATCTC | |
| | | AACATGAGTAATT | AAGAAGGTATAGGC | |
| | | TGATGGGGGCTAT | CTTTGTTTGTAAGTC | |
| | | TATGAACTGAGAA | CTGCTGTCCTAGCA | |
| | | ATGAACTTTGAAA | TCCTATAATCCTGG | |
| | | AGTATCTTGGGGC | ACTTCTCCAGTACTT | |
| | | CAAATCATGTAGA | TCTGGCTGGATTGG | |
| | | CTCTTGAGTGATG | TATCTGAGGCTAGT | |
| | | TGTTAAGGAATGC | AGGAAGGGCTTGTT | |
| | | TATGAGTGCTGAG | CCTGCTGGGTAGCT | |
| | | AGGGCATCAGAAG | CTAAACAATGTATT | |
| | | TCCTTGAGAGCCT | CATGGGTAGGAACA | |
| | | CCAGAGAAAGGCT | GCAGCCTATTCTGC | |
| | | CTTAAAAATGCAG | CAGCCTTATTTCTAA | |
| | | CGCAATCTCCAGT | CCATTTTAGACATTT | |
| | | GACAGAAGATACT | GTTAGTACATGGTA | |
| | | GCTAGAAATCTGC | TTTTAAAAGTAAAA | |
| | | TAGAAAAAAAACA | CTTAATGTCTTCCTT | |
| | | AAAAAGGCATGTA | TTTTTTCTCCACTGT | |
| | | TAGAGGAATTATG | CTTTTTCATAGATCG | |
| | | AGGGAAAGATACC | AGACATGTAAGCAG | |
| | | AAGTCACGGTTTA | CATCATGGAGGTAA | |
| | | TTCTTCAAAATGG | GTTTTTGACCTTGAG | |
| | | AGGTGGCTTGTTG | AAAATGTTTTTGTTT | |
| | | GGAAGGTGGAAGC | CACTGTCCTGAGGA | |
| | | TCATTTGGCCAGA | CTATTTATAGACAG | |
| | | GTGGAAATGGAAT | CTCTAACATGATAA | |
| | | TGGGAGAAATCGA | CCCTCACTATGTGG | |
| | | TGACCAAATGTAA | AGAACATTGACAGA | |
| | | ACACTTGGTGCCT | GTAACATTTTAGCA | |
| | | GATATAGCTTGAC | G | |
| | | ACCAAGTTAGCCC | (SEQ ID NO: 196) | |
| | | CAAGTGAAATACC | | |
| | | CTGGCAATATTAA | | |
| | | TGTGTCTTTTCCCG | | |
| | | ATATTCCTCAGGT | | |
| | | ACTCCAAAGATTC | | |
| | | AGGTTTACTCACG | | |
| | | TC | | |
| | | (SEQ ID NO: 195) | | |
| CIITA | CIITA | ACAGGAGGTAACC | AGCCCCAAGGTAAA | CCTTG |
| ex 1 | gRNA1 | ATTTAACAAGAAA | AAGGCCGGGAAAGC | GGGCT |
| | exon 1 | GCAGAGTGATGTT | ATCTTAATTTAGCGT | CTGAC |
| | | AGATTATAGCAAG | GCAGTCTCAGCTGG | AGGTA |
| | | ATACTGTTGACTG | TCCTGCCATTCCAG | (SEQ ID |
| | | TAGAAGGCTCTGA | ATAAACAGAGAAAC | NO: 219) |
| | | GGCTAGAGAGCTG | CATTCTGAATTGGG | |
| | | CTTTCTATAAAAC | GATGGGGGTGAGGA | |
| | | AGAGTGATCATAT | TGGGAACAGGAGTC | |
| | | ATTAGAAGAGGTG | TGTGTCCTGCTGGG | |
| | | TTAAAGACATGTT | GCAGGCCATTGGAA | |
| | | CACACCAAGCTGA | GATGTGAAAGAGTT | |
| | | GACTTCCTCCTTG | GTCTATTTCCTTCCA | |
| | | ATACCACCAGGAG | CCGGAGGGAGACTT | |
| | | GATGGGCAGAGAC | CAGGTCAGCCAGGT | |
| | | TGGAAAAGACACT | GTCTGGAGTATGAA | |
| | | AACTTTCTCCCTAT | CCATGTATCAGCAC | |
| | | GGGAGTCAGTATT | CGAAAGGTTCTAGA | |
| | | ATTTAGCATCACT | AGTCAGACTTTCGG | |
| | | TTGGCGGGTCACC | GCAGTGTGTCACTA | |
| | | CCAAACCATCTGA | ACTCTCAGCATGCT | |
| | | CTACAAGGGTACC | GGCCTGGCTCGGCC | |
| | | ATATTTGGGTTAA | CACAGCAAGGTCTT | |
| | | CACTCTTTTGGTAT | CTCGCCTCCCTTTGG | |
| | | AATTTATGTTTTAG | GTAAATACTGAGGG | |
| | | TCCAATGTCTTGG | GTGCCTCTGCAGGA | |
| | | GATGAAAATGACA | CGGGACCTCTGCCA | |
| | | GGTGGGCCACTTA | GACTCCACTCCATA | |
| | | TGATCTCCAGAGA | CCCAGAGAAGCAGG | |
| | | AATTCAGGGCAAT | GAAACCAAAATTGG | |
| | | TTGGTGTGGGAGT | AGTCAGCCTTGAGG | |
| | | AGGCATGGTAGAG | TGTAGCTGTTGAGC | |
| | | GAGAGCAGCATCT | CCTCAGCAGCTGGG | |
| | | AAGAAGTCCCCAG | GAGAGCTGGCGGAT | |
| | | CAGAGGCTCTCAG | GCTGCCCTCCCCCC | |
| | | CTTGTCTTGAGGC | AGTTTCCTAATGGT | |
| | | ATCTGGGCGGAGG | GTTGTTTAAAAAGG | |
| | | GCTATGATACTGG | GTCAGGGGACGGGG | |
| | | CCCCATCCTGCAG | GAACAGATGGTGGG | |
| | | AAGGTGGCAGATA | AAGAGCACAGTGCA | |
| | | TTGGCAGCTGGCA | GACACCTGGCACCG | |
| | | CCAGTGCGGTTCC | GCTCTGAAGGCAGC | |
| | | ATTGTGATCATCA | ATGGCAGCTACACC | |
| | | TTTCTGAACGTCA | GTTGGCTGGGAAGG | |
| | | GACTGTTGAAGGT | GTGTGCCCCTGAAG | |
| | | TCCCCCAACAGAC | AAGTCGTTTACATT | |
| | | TTTCTGTGCAACTT | CTCGAGTCAATTTTC | |
| | | TCTGTCTTCACCA | CTGGAGTGTACAAT | |
| | | AATTCAGTCCACA | GGACCTGTGGGAAA | |
| | | GTAAGGAAGTGAA | GCCTGTATGAAAGG | |
| | | ATTAATTTCAGAG | GTAATGATGAGGGA | |
| | | GTGTGGGGAGGGC | CCTAGCACAGTGTC | |
| | | TTAAGGGAGTGTG | CAATATTTTATAGG | |
| | | GTAAAATTAGAGG | AACTGGAATTGAGC | |
| | | GTGTTCAGAAACA | TCATAGGAGCTCAA | |
| | | GAAATCTGACCGC | TTTTATTGGCATTGC | |
| | | TTGGGGCCACCTT | TGTTGTTGGATGGTT | |
| | | GCAGGGAGAGTTT | AAAGGGGTGGTATC | |
| | | TTTTGATGATCCCT | CCTTTTCTCAGACTC | |
| | | CACTTGTTTCTTTG | CCCTGAAATGTATG | |
| | | CATGTTGGCTTAG | GTTTGCTTTGAACCC | |
| | | CTTGGCGGGCTCC | AGAGACTGATGACA | |
| | | CAACTGGTGACTG | GGTCTGCCGGTGTG | |
| | | GTTAGTGATGAGG | GTTGGGTGCAGCCT | |
| | | CTAGTGATGAGGC | TAAGTTGCTACGGG | |
| | | TGTGTGCTTCTGA | AAAGTGTTGGAGGG | |
| | | GCTGGGCATCCGA | GGAGAAGTCAGAGG | |
| | | AGGCATCCTTGGG | TAACCTTGCCCCCTC | |
| | | GAAGCTGAGGGCA | CCTCAATTCCAGAT | |
| | | CGAGGAGGGGCTG | GAGGAAATTCAGGC | |
| | | CCAGACTCCGGGA | CTGAAAAGGGA | |
| | | GCTGCTGCCTGGC | (SEQ ID NO: 198) | |
| | | TGGGATTCCTACA | | |
| | | CAATGCGTTGCCT | | |
| | | GGCTCCACGCCCT | | |
| | | GCTGGGTCCTACC | | |
| | | TGTCAG | | |
| | | (SEQ ID NO: 197) | | |
| CIITA | CIITA | AAACTTCTACCAC | AAGTTGGGCAGAAA | TGCCC |
| ex5 | gRNA4 | CGTCACCTATCTCT | AGTCAGAAAAGACG | AACTT |
| | exon 5 | CAGGGTTTCCTAA | TGAGTGAGCCCCTC | CTGCT |
| | | ACATACTCTGAAC | CCTGATCCAACCTA | GGCAT |
| | | AAGTTTCCTCACT | GCCTTGCTTGAGAC | (SEQ ID |
| | | CTGCCACTGTGAC | CTGGCCTTTCCTTGA | NO: 220) |
| | | CCAGAAAGCTATC | CTCCAAAGCCTGCT | |
| | | TGTTCTCCTTCTCC | GTGGGTCCAACTTG | |
| | | CAGACTCTGCCTC | CTTCCCTCGCTAAGT | |
| | | ATCTTTCAAGGCC | CCTGTCTGGTTGGG | |
| | | TGGTTCCAGGATC | AGGCCCTTTAAAAG | |
| | | CCTTCCTCCAGGA | CCAACAGGAGCCTT | |
| | | AGCCTTCCCTGAT | AAAATGTACATCTG | |
| | | TGCCCTATTCCAC | ATTATTTCATGGCCC | |
| | | CATACACCTTTTTT | TGATAACCCTCCAA | |
| | | CTCGGACTTCATG | TGGCTACAAAATAC | |
| | | TCATGGTGCCTAT | ATGCCACAAGGCCT | |
| | | ATTCCAAGGGCTC | GTATGGCCCTTCCTC | |
| | | TGAGCCATGTACC | CCTCTCCAAACCCA | |
| | | CCTTTATATAGTTA | CTATGAGACACCAC | |
| | | CCACTATTTACTG | ACTTCAGCCACCAC | |
| | | AGTGCCTACTGTA | CAGCTTCTCCACTCC | |
| | | TACCAGCTACTGT | TATAACCCATGTGC | |
| | | GTTGGATGCTCTA | CCTTTCAAGCCTCG | |
| | | GATGTAGAACCTC | GGGCCTTTGCAGTT | |
| | | TAATTATCACCAT | GCTATAGTCTCTATC | |
| | | CGATTCCTGGGTA | TGGAATGCCCTTCC | |
| | | GTAGGCATTATTT | CCCAGTTCTTCCCAT | |
| | | ATTCATCTTACAC | GGCTGACTCCTTTG | |
| | | AGATGAGAAAATG | AATCTTTCTGGTGTT | |
| | | GAGGCCCACAGTG | GGCTAAACTGTCAC | |
| | | GTTAAATAAGTAG | CTCTTCCTGGAACC | |
| | | CCCAAGATTGCAC | CTTCTCTGACCATCC | |
| | | AGCTAGTAGGGCT | TTCCATGTAGATTA | |
| | | AGTGGAAAGTAGA | GCTCAGTTATTCTCA | |
| | | GGTGGAATTTGAA | CCTTGTGTGTCTTTT | |
| | | CTCAAATCCTGCA | TCCTTGCAGTTTAGC | |
| | | GTAACTCTACCAT | ACTCATTACCATCT | |
| | | TCTGCCTTGCTCTT | GGACATATTTTACG | |
| | | CTTTGTAGCAGTA | CCTTGCTCTCCCACT | |
| | | GATAAGTTTTCAT | GTGAGGACAGGGAC | |
| | | GGATACATACCTC | CTTGTCTTTCTTGCT | |
| | | ATCCTTTTGATTAG | CGTGACTGTTTCCCC | |
| | | ATTAAGGGCCCCT | AGCATCTAGTGCAG | |
| | | GGAGTGTCAGTGT | TGCCTGGTATGCAG | |
| | | TCATTCATTTGTTT | TAGCACCTCAGTAG | |
| | | GATCATTCATTCA | ATATCTGTTGAATG | |
| | | TTCAACAAACATT | AAAACATCTGTAAA | |
| | | TCTTGAGTCCCCA | ATGGGTGTAACAGT | |
| | | CTGTGTGCCAGGC | TAACTGAGTACTTA | |
| | | CCAGAGGTTCCCC | TTATGGGTCTGACC | |
| | | AGCCCAAGGCCTG | ATGTGTAAGTCCTG | |
| | | GCACACAGTGGGC | TATCTATTTATTCAG | |
| | | CTTCAGTTAGACC | TTCTTAAACAGGTG | |
| | | TTGTTGATTGACT | AATCGCACACAGGG | |
| | | GCGCTTTTCCTTGT | TATGAGATTTTAAA | |
| | | CTGGGCAGCGGAA | AGTGCAAAGAATAT | |
| | | CTGGACCAGTATG | TCAGTGAAGGCTGG | |
| | | TCTTCCAGGACTC | GCGCAGCGGCTCAC | |
| | | CCAGCTGGAGGGC | ACCTGTAATCCCAG | |
| | | CTGAGCAAGGACA | CAGTTTGGGAGGCC | |
| | | TTTTCAGTAAGTTT | AAGGGGGACGGATC | |
| | | GTGGTGGGTGGGG | ACTTGAGGTCAGGA | |
| | | AGGTCTTGGCTCA | GTTTGATACCTGCCT | |
| | | GCCTGCATTTCCT | GGCCAACATGGTGA | |
| | | GCCTTGTTCCCTG | AACCGCGTCTCTAC | |
| | | GGGGGTGCCCTAA | CAAAAAATACAAAA | |
| | | TACCTGACGACCA | ATTAGCCGGGTGTG | |
| | | TTCATTGATGGGC | GTGGTGCACGCCTG | |
| | | AGTCAGACCCCTC | TAATCCCAGCTACT | |
| | | TCCCCAAGGTGGG | CGGGAGGCTGAGGC | |
| | | TACAATAGAGACT | AGGAGAATCGCTTG | |
| | | CACCTTGGGCTTT | AACCCAGGAGGTGG | |
| | | CATTGATTGTGTG | AGGTTGCAGTGAG | |
| | | AGTTGGTCTCTGG | (SEQ ID NO: 200) | |
| | | TTTTTCTCAAAGTA | | |
| | | GAGCACATAGGAC | | |
| | | CAGATGAAGTGAT | | |
| | | CGGTGAGAGTATG | | |
| | | GAGATGCCAGCAG | | |
| | | (SEQ ID NO: 199) | | |
| CD70 | CD70 | AAAAATAAAAAAT | CGCGGGCTTGGTGA | GTCCC |
| | gRNA1 | AAAAATATTAACT | TCTGCCTCGTGGTGT | ATTGG |
| | exon 1 | TAATTTAACTTTA | GCATCCAGCGCTTC | TCGCG |
| | | AACAAAAAAGCA | GCACAGGCTCAGCA | GGCTT |
| | | GGTGGTCTCCAAG | GCAGCTGCCGCTCG | (SEQ ID |
| | | AATGCAGGAGATA | AGTCACTTGGGGTG | NO: 221) |
| | | ACTGACCGGGTGC | AGTTGAGATGGAAA | |
| | | AGTGTCTCATGCC | AGTTGGGAAGAAAA | |
| | | TTTAATCCCAGCA | CATAGAGAGGCGCG | |
| | | CTTTGGAAGGCCA | TGACCGAAAAGACA | |
| | | AGGCGGGTGGATC | GAATGAGATGGGTA | |
| | | ACCCAAGGTCAGG | CAAAGAGGCCAGAG | |
| | | AGTTCAAGTCCAG | AGGAAGATCTGGTA | |
| | | CCTGGCCAACATG | GGGCAGAGACAGA | |
| | | GTGAAACCCCATC | GACCAGAACAGGGA | |
| | | TCTACTAAAAATA | GGCGAGGCGGGGAC | |
| | | CAAAAAATTAGCC | CAGGCTGCCCGGTG | |
| | | AGGCATGGTGGCG | TAGGGGCTACGAGA | |
| | | CGCGCATGTTACT | CAGGCAGCCCTGCC | |
| | | CCCAGCTACTCGC | AGGAGGTACAGGGA | |
| | | GAGGCTCAGACAG | GATCCCGGGATGGG | |
| | | GAGAATCGCTTGA | AAAGGTAGGCACAC | |
| | | ACCCAGGAGATCG | ATGGAAATGGAAGA | |
| | | AGGTTGCGGCGAG | TGACTCGGCTCTGG | |
| | | CTGAGATGGCGCC | TGTTCCCCCGGCAG | |
| | | ACTGCACTCCAGC | GCTGACTCAGAGGC | |
| | | CTGGGTGACAGAG | TGCTGGGGGCTTCA | |
| | | GGAGACCTCCGTC | CAAGGCTGGGCGTG | |
| | | TCAAAAACAAAAC | GGGGCTTCCTGGGG | |
| | | AAATCAAAAAAAT | CCTCCTAGGACGGG | |
| | | GCAGGAGAGGGGT | ATGGCCCCAGCCAC | |
| | | ACACGAATATTTG | TCGCTCCGGGTGGG | |
| | | GGGAGCACCCCCA | GGAGGGGTCCCTTT | |
| | | ATTCTTGGATGTCT | GGGGACCGCGCCGG | |
| | | GCTGTATCCCCAG | GCGCCTTTGCAGCG | |
| | | TGCACAGCACAAT | TAGAGAGTCCGCTG | |
| | | CTAATCCCTAATA | CGCGCGGTGCTCTC | |
| | | AATGTGCAGTGGA | GCGCCCAGTGACAT | |
| | | GGTTTGTTGAATA | CCAGGAAAACGATT | |
| | | AATGAATGGGCCC | CGGGAAACGAAGA | |
| | | CAGAAGAATGAGG | AGTTCTTTTGAAGG | |
| | | TGGAGAGGGGAAT | TCTCGACTTCACGTT | |
| | | AGGAAGATTGAAT | CCCCGCTGGTTCAG | |
| | | GTCTCCTGCCTGA | ACCTGCTTCCTCTTT | |
| | | AGGTCGGGCGGGG | AAGAAGTCTTAAGA | |
| | | AGGGGTTGGGGGC | GTAAAAAAAAATAA | |
| | | AGGCAACTCTGAG | AATGAAATAAAATC | |
| | | GCTCACCCGGGGC | ACCAGTGCGCGCCG | |
| | | CACTGCCTGCATC | TGGGATGAGAGGTG | |
| | | CTGGCAACTGCCT | GAAAGGAGGATGG | |
| | | CCACCCACTTTAG | ACAGAGAAAAGAG | |
| | | GATCTTCAGACTG | AGCTCCTGGCACAG | |
| | | GCAGCGGTTGGAG | GGGACACATAGAAC | |
| | | GGAATTTCCCCTC | CTCTCTGCTTACGTC | |
| | | GCCAATTGCTCAA | CGTGCCCTGTTTTCT | |
| | | GTCCCTCCCCTCG | GGTCTTTTCTTCCAG | |
| | | ACCGGCCGGACAT | TGGGACGTAGCTGA | |
| | | CCCCAGAGAGGGG | GCTGCAGCTGAATC | |
| | | CAGGCTGGTCCCC | ACACAGGTAACACG | |
| | | TGACAGGTTGAAG | GGGGACGTGGAGGG | |
| | | CAAGTAGACGCCC | ACGGGGAGAAGAA | |
| | | AGGAGCCCCGGGA | GAGGCACAGAGAG | |
| | | GGGGGCTGCAGTT | AGAAGGAAGGAGA | |
| | | TCCTTCCTTCCTTC | GGTAGAAAGACAAG | |
| | | TCGGCAGCGCTCC | TGGGGAGAGACAGA | |
| | | GCGCCCCCATCGC | GAGAAAGAGACAC | |
| | | CCCTCCTGCGCTA | AGACAGAGACGGA | |
| | | GCGGAGGTGATCG | GGGAGAGAGGGAG | |
| | | CCGCGGCGATGCC | GGAGAGATAGGGA | |
| | | GGAGGAGGGTTCG | GGGAAACGGAGAG | |
| | | GGCTGCTCGGTGC | GGGGAGACAGAGA | |
| | | GGCGCAGGCCCTA | GAAGACAGAGAGG | |
| | | TGGGTGCGTCCTG | (SEQ ID NO: 202) | |
| | | CGGGCTGCTTTGG | | |
| | | TCCCATTGGT | | |
| | | (SEQ ID NO: 201) | | |
| CLYBL | CLYBL | GTCCAGACTCAGG | ATCATGTCTGCTCG | GTCAG |
| | gRNA2 | AGGACTTAGTTCT | ACAGCTCTGACTAA | AGCTG |
| | intron 2 | CTTCATAAAAAGT | ACACTGTGCCCCAA | TGATC |
| | | TGATCACCTTTGCT | AGTGTTGAGGAATT | ACTCT |
| | | TTCTCCTTTGCATG | GGGAAAACCTAGCT | (SEQ ID |
| | | GGCAATAAAGAAG | GAGTTAGTGGTCTC | NO: 222) |
| | | TGAAAAATAAATT | TTTTCTGTTACAATA | |
| | | CCCAGTGAGCTTG | AAGCTCATAATGAA | |
| | | CATTCTGCTTGGG | AATTAGCCTTCTTTG | |
| | | AACAACATAGCAC | TTCTTCCCCAAGTCT | |
| | | ATCCATTTTCTAG | TTCTTTCTAGACGA | |
| | | AGAGCTGTCCAGT | AACTACTTTCAACT | |
| | | CCCCATTTGAGGG | GTTTTAACTTCCTTA | |
| | | CTGCTAGCCACAT | CTGTTAACTTCCATA | |
| | | GTCAAGCAGGAAC | TTTCTAGATAGTAT | |
| | | CCAGAGTATAACC | GGTCAGACTGTTAT | |
| | | AGGAAAATGTCTG | TTCTTGACTTTTAAA | |
| | | TGGTAGACCCCAG | TGTAAGATATTATC | |
| | | TGATTCATGCCTC | TACTGACTTCCTTCT | |
| | | CCATCTCCACACC | ATGTAAGATGAGGA | |
| | | CTCCTGTAGTCCC | TTGAGCTCTCTTACC | |
| | | CTCCCACGCTGAT | CTTCTCCCATTTCCT | |
| | | TCTGGACTAGGCC | CATCCTTCCAACAT | |
| | | ACATGGCTTGGCC | AAATATATTTTGGG | |
| | | AGTGGAACACATG | ATTATATCAACATT | |
| | | CAGGCTTGCACGT | CAATGTTACTTAAA | |
| | | CTGGAACTCTGCC | GTGACCTTGTAAAT | |
| | | ACCTAATTTAGGA | ATTTTCACAACTGA | |
| | | TCCCAGACTCTCC | GCCATGTTTGATTTG | |
| | | TACTGGAGACACA | TATACTTATGTTTAC | |
| | | GGTCCTTAGTGAC | TTTACTGTTTTTCCT | |
| | | AGTCTGCACCACC | GAAGTTAATAATTG | |
| | | ATTCAGACAAGTC | CCTTGAATTTATTTA | |
| | | AGTAGGGCCATCT | TTTCTTTAAAAATGT | |
| | | TAGATCATCCAGC | TTCATTACTCAGGA | |
| | | CCTAGTCAAGCCA | CTGTAGTTTACATTA | |
| | | CCAGATAACTGTA | CGATTCTTTGTGTTA | |
| | | CCCACATAAGTGA | TACAGTTGATGGGT | |
| | | CCCCTGGCGAGAC | TTCTTTTCTTTCTTA | |
| | | CAGCAGGAGAATC | ATTTCTTTAAAAAA | |
| | | ATGCCAATGGGCC | TAGAGATGGGGTCT | |
| | | AATATACATTCTG | TACTATATTACCCA | |
| | | ACCCACAGTTTCA | GGCTGGTCTTGAAG | |
| | | TAATAAAATAAAA | TCCTGGGCTCAAGT | |
| | | TGGTTGTGGTTGT | GATCTTCCTGTCTCA | |
| | | AAGCCACTATGTT | GCCTACCAAGTAGC | |
| | | TCAGAGTGGTTTG | TGAGACTATAGGTG | |
| | | TTACACAGCAATA | CAAAAAAGCCACTA | |
| | | AATAACTAATATA | TACCTGGCTAGTTT | |
| | | GTAGGCATACCAT | ACAGGTTTTAACAA | |
| | | CAAGTCCAAAGTA | ATGCATTATGCCAC | |
| | | GGTAGAGAAGAAT | GTATCCATTATTAC | |
| | | GTAAATAGCAGAG | AGGATCACACAAGA | |
| | | CAAAACAGCATGA | TATTTTCATTACCCT | |
| | | CTGGTGGCTGGGA | GAAAGCATCCCTGT | |
| | | GGCTTAAAACTGG | GTTCCACCAATTCA | |
| | | GACAGGATCAGAG | TCCTGCCTCCATGA | |
| | | TCATGAAAGAAGT | GCCGCTGGCAACCA | |
| | | CAAAGAAATGGTT | CTGATCTCTATAGTT | |
| | | CAGAAGTAAGGCT | TTGCCTTTTCTAAAA | |
| | | GAGACTGACTTAC | TGTCATATAATTGG | |
| | | AAAAGCTGAAAGT | AATCATACAGTCTG | |
| | | CCCTTTAAGTTGG | TAGCATTTTCAGAC | |
| | | TGTTTGGTGCATT | TAGCTTTTAAAATTT | |
| | | GGCAGGGGCAGGT | GGCAATATGCATTT | |
| | | ATGGTGACTTAAA | AAGGTTCCTCCTTA | |
| | | AGAGCCATGCTCA | AATGTGAAGGGCAT | |
| | | ACAAGATCAAGCA | AGCCAATGTGGCTT | |
| | | CAACACAATCACG | GATAGCTCATTTCTT | |
| | | GGTCACCCCAGCA | TTTATTGGTGAATAT | |
| | | GACCTTAGCGAGT | TTCATTGTCTGGATG | |
| | | CTAGCCATTTCTTT | TTCCACAGTTTGTTT | |
| | | GGTGGTGGTCACA | ATCCTCGAGAGCTT | |
| | | GTCATGCTTCAGC | GGCGT | |
| | | CCAGTTTCCACTT | (SEQ ID NO: 204) | |
| | | GGACAAATGGTAC | | |
| | | ATATTTTCAATGA | | |
| | | GATGAAAATTAAG | | |
| | | ATACAATCCATGT | | |
| | | GCTCAGAGAGTGA | | |
| | | TCA | | |
| | | (SEQ ID NO: 203) | | |
| NKG2A | NKG2A | TTATGTAGTGTTCC | AATCTGCCCCCAAA | ACTCA |
| | gRNA1 | TAATACTATTTAG | CCCAAAGAGGCAGC | GACCT |
| | exon 3 | GTTATGCATAAGT | AACGAAAACCTAAA | GAATC |
| | | TAACTGTGTCACA | GGCAATAAAAACTC | TGCCC |
| | | TAGCATTTTCTGTC | CATTTTAGCAACTG | C |
| | | ATATACAAATATT | AACAGGAAATAACC | (SEQ ID |
| | | TAATTTTTTATAAT | TATGCGGAATTAAA | NO: 223) |
| | | TTTTAATAAATAA | CCTTCAAAAAGCTT | |
| | | TTTTTAATAATTTC | CTCAGGATTTTCAA | |
| | | TTAGAACATTTCA | GGGAATGACAAAAC | |
| | | GCCATTAAGAAAA | CTATCACTGCAAAG | |
| | | GACCATTTAATTA | GTAAAGCATTTAAA | |
| | | CCTCTATTGATTTT | AGATCCTCAATATA | |
| | | CATTAATGTGTAT | ACAGTCTAGGATGT | |
| | | AAATTACTAATTA | GCAGCTTGGGGTAC | |
| | | AAAGTAATGTAGT | AGGAATGTGGGGAA | |
| | | GCAAGAAATTTTT | AGAGAAGGGAGTGC | |
| | | ATATATATATATG | TCATATATCTTCTAT | |
| | | TCAACTTTACTATT | TTGCAAAGATCAGA | |
| | | ACTCATGTGCAGA | ATTCCAAGTTGAGA | |
| | | CCACATAGTCTTA | TATGCTATTTCAATG | |
| | | ACCATTACTGGAT | TAAAGTATGAAGAC | |
| | | TTCAAAATTAACA | TGATTGAACTCATT | |
| | | TAAAAATGAGTGC | GTTGAAGTTTGTAG | |
| | | AGAAGTTTTTCTA | TCTTTGTCAAATAAT | |
| | | ATACAACATTTTA | TCATGGAGCATTAT | |
| | | ATTTTTAACACAG | TTTTCCTGAAAATTC | |
| | | GTAAAACATCAGA | AATGGTATATTATT | |
| | | CTTTAAGAAATAT | CTGAGAAAAAGATT | |
| | | ATTTTTATTCCTGA | ACAATGGGAGATGA | |
| | | ACTTCTTTATTCCT | GGGTTTGGGGTCCA | |
| | | TAGTAATTTATTG | AGTTTCTCTGTATGA | |
| | | CTTCTCATTGCCCC | TTCCTGTGCATTCAG | |
| | | AGCAATAATATTT | GTTCTCTTGTCTGTG | |
| | | TGTCAAATGCAGA | AATCTTCTAAACGA | |
| | | AAATTTATCTTTTT | CTGTATCCACCTCTC | |
| | | TTTTTGAGTCGGA | CTTTCGCACTGTTCC | |
| | | GTCTCACTCTGTC | CATTTCTCTCCCTGC | |
| | | GCCCAGGCTGGAG | AGATTTACCATCAG | |
| | | TGCAGTGGCACAA | CTCCAGAGAAGCTC | |
| | | TCTCCGCTCACTG | ATTGTTGGGATCCT | |
| | | CAACCTCTGCCGC | GGGAATTATCTGTC | |
| | | CCATGTTCAAGAG | TTATCTTAATGGCCT | |
| | | ATTCTCCTGCCTCA | CTGTGGTAACGATA | |
| | | GCCTCCCGAGTAG | GTTGTTATTCCCTGT | |
| | | CTAGGACTACAGG | AAGTCTATTTTCGA | |
| | | CGCCTGACATCAT | AGATTACAAGGGGA | |
| | | GCCCGACTACTTT | ATTTTCACGTTAATG | |
| | | TTGTATTTTTAGTA | ATTGAATGTGCCTC | |
| | | GAGACGGAGTTTC | TAAACATTTCATATT | |
| | | ACCGTGTTAGCCA | TTCAGGGAATAGAG | |
| | | GGATGGTCTCGAT | TTCTCATTGTAATGT | |
| | | CTCCTGACCTCGT | ATATATTTGGACTA | |
| | | GATCGGCATGCCT | AATGTGGAATGATT | |
| | | CGGCCTCCCAAAG | ATTCTGAATTTGTCA | |
| | | TGCTGGGATTACA | AAGAATAAATGAAA | |
| | | GGCGTGAGCCACC | GAATAATTGTTGAA | |
| | | GCGCCCGGCCTAA | AGTATTCGCTTCTG | |
| | | AAATCTTTTTTTAA | ATGCAATCGTATGT | |
| | | AACAAATATTCAT | ATATATTTGGATTTC | |
| | | AAGAAACGTGTTT | ATAACTCAAAAATA | |
| | | AGGCTTGAAGAAA | TGTTCTAGGAGTCT | |
| | | ATCAGAGAAAGAA | GAAAAACCTTACTG | |
| | | CTTTAGATTATTTA | AGAAATAGAAATTA | |
| | | ATGCAAAATGAGC | ATTTTTGAAAGTAG | |
| | | TCCAATACTCGTT | TTAAATCAAGAATT | |
| | | CTCCACCTCACCC | ATAAGAACTATATG | |
| | | TTTTAATTGCACTA | AGATGGTGAAATTT | |
| | | GGGAATCCTGTAT | GGTTCTTTAGATCTA | |
| | | ATAAACCATTTAT | TGAAATACTTTTCC | |
| | | TAACTTCTTAACT | AAAAAACCACCATT | |
| | | ACTGTTATTATAG | ACTTTATC | |
| | | AGTACAGTCCCTG | (SEQ ID NO: 206) | |
| | | ACATCACACACTG | | |
| | | CAGAGATGGATAA | | |
| | | CCAAGGAGTAATC | | |
| | | TACTCAGACCTG | | |
| | | (SEQ ID NO: 205) | | |
| TRAC | TRAC | CTCAAAAGGCAGG | TGAGAGACTCTAAA | GAGTC |
| | gRNA1 | AGGTCGGAAAGAA | TCCAGTGACAAGTC | TCTCA |
| | exon 6 | TAAACAATGAGAG | TGTCTGCCTATTCAC | GCTGG |
| | | TCACATTAAAAAC | CGATTTTGATTCTCA | TACAC |
| | | ACAAAATCCTACG | AACAAATGTGTCAC | (SEQ ID |
| | | GAAATACTGAAGA | AAAGTAAGGATTCT | NO: 224) |
| | | ATGAGTCTCAGCA | GATGTGTATATCAC | |
| | | CTAAGGAAAAGCC | AGACAAAACTGTGC | |
| | | TCCAGCAGCTCCT | TAGACATGAGGTCT | |
| | | GCTTTCTGAGGGT | ATGGACTTCAAGAG | |
| | | GAAGGATAGACGC | CAACAGTGCTGTGG | |
| | | TGTGGCTCTGCAT | CCTGGAGCAACAAA | |
| | | GACTCACTAGCAC | TCTGACTTTGCATGT | |
| | | TCTATCACGGCCA | GCAAACGCCTTCAA | |
| | | TATTCTGGCAGGG | CAACAGCATTATTC | |
| | | TCAGTGGCTCCAA | CAGAAGACACCTTC | |
| | | CTAACATTTGTTTG | TTCCCCAGCCCAGG | |
| | | GTACTTTACAGTTT | TAAGGGCAGCTTTG | |
| | | ATTAAATAGATGT | GTGCCTTCGCAGGC | |
| | | TTATATGGAGAAG | TGTTTCCTTGCTTCA | |
| | | CTCTCATTTCTTTC | GGAATGGCCAGGTT | |
| | | TCAGAAGAGCCTG | CTGCCCAGAGCTCT | |
| | | GCTAGGAAGGTGG | GGTCAATGATGTCT | |
| | | ATGAGGCACCATA | AAAACTCCTCTGAT | |
| | | TTCATTTTGCAGGT | TGGTGGTCTCGGCC | |
| | | GAAATTCCTGAGA | TTATCCATTGCCACC | |
| | | TGTAAGGAGCTGC | AAAACCCTCTTTTTA | |
| | | TGTGACTTGCTCA | CTAAGAAACAGTGA | |
| | | AGGCCTTATATCG | GCCTTGTTCTGGCA | |
| | | AGTAAACGGTAGT | GTCCAGAGAATGAC | |
| | | GCTGGGGCTTAGA | ACGGGAAAAAAGC | |
| | | CGCAGGTGTTCTG | AGATGAAGAGAAG | |
| | | ATTTATAGTTCAA | GTGGCAGGAGAGGG | |
| | | AACCTCTATCAAT | CACGTGGCCCAGCC | |
| | | GAGAGAGCAATCT | TCAGTCTCTCCAACT | |
| | | CCTGGTAATGTGA | GAGTTCCTGCCTGC | |
| | | TAGATTTCCCAAC | CTGCCTTTGCTCAG | |
| | | TTAATGCCAACAT | ACTGTTTGCCCCTTA | |
| | | ACCATAAACCTCC | CTGCTCTTCTAGGCC | |
| | | CATTCTGCTAATG | TCATTCTAAGCCCCT | |
| | | CCCAGCCTAAGTT | TCTCCAAGTTGCCTC | |
| | | GGGGAGACCACTC | TCCTTATTTCTCCCT | |
| | | CAGATTCCAAGAT | GTCTGCCAAAAAAT | |
| | | GTACAGTTTGCTTT | CTTTCCCAGCTCACT | |
| | | GCTGGGCCTTTTTC | AAGTCAGTCTCACG | |
| | | CCATGCCTGCCTTT | CAGTCACTCATTAA | |
| | | ACTCTGCCAGAGT | CCCACCAATCACTG | |
| | | TATATTGCTGGGG | ATTGTGCCGGCACA | |
| | | TTTTGAAGAAGAT | TGAATGCACCAGGT | |
| | | CCTATTAAATAAA | GTTGAAGTGGAGGA | |
| | | AGAATAAGCAGTA | ATTAAAAAGTCAGA | |
| | | TTATTAAGTAGCC | TGAGGGGTGTGCCC | |
| | | CTGCATTTCAGGT | AGAGGAAGCACCAT | |
| | | TTCCTTGAGTGGC | TCTAGTTGGGGGAG | |
| | | AGGCCAGGCCTGG | CCCATCTGTCAGCT | |
| | | CCGTGAACGTTCA | GGGAAAAGTCCAAA | |
| | | CTGAAATCATGGC | TAACTTCAGATTGG | |
| | | CTCTTGGCCAAGA | AATGTGTTTTAACTC | |
| | | TTGATAGCTTGTG | AGGGTTGAGAAAAC | |
| | | CCTGTCCCTGAGT | AGCTACCTTCAGGA | |
| | | CCCAGTCCATCAC | CAAAAGTCAGGGAA | |
| | | GAGCAGCTGGTTT | GGGCTCTCTGAAGA | |
| | | CTAAGATGCTATT | AATGCTACTTGAAG | |
| | | TCCCGTATAAAGC | ATACCAGCCCTACC | |
| | | ATGAGACCGTGAC | AAGGGCAGGGAGA | |
| | | TTGCCAGCCCCAC | GGACCCTATAGAGG | |
| | | AGAGCCCCGCCCT | CCTGGGACAGGAGC | |
| | | TGTCCATCACTGG | TCAATGAGAAAGGA | |
| | | CATCTGGACTCCA | GAAGAGCAGCAGGC | |
| | | GCCTGGGTTGGGG | ATGAGTTGAATGAA | |
| | | CAAAGAGGGAAAT | GGAGGCAGG | |
| | | GAGATCATGTCCT | (SEQ ID NO: 208) | |
| | | AACCCTGATCCTC | | |
| | | TTGTCCCACAGAT | | |
| | | ATCCAGAACCCTG | | |
| | | ACCCTGCCGTGTA | | |
| | | CCAGC | | |
| | | (SEQ ID NO: 207) | | |
| CD38 | CD38 | CTTGCTGCTCTGG | TGTCCGGGGACAAA | CCGAG |
| | gRNA2 | AACAGGCCAAGAG | CCCTGCTGCCGGCT | ACCGT |
| | exon 1 | CTTTTCTGCCTCAG | CTCTAGGAGAGCCC | CCTGG |
| | | AGTCTTTGCACCT | AACTCTGTCTTGGC | CGCG |
| | | GCCATTTCCTCTGC | GTCAGTATCCTGGT | (SEQ ID |
| | | TTGGGAAATGTTT | CCTGATCCTCGTCGT | NO: 225) |
| | | GCCCCAAGGGAGT | GGTGCTCGCGGTGG | |
| | | TGGGTGACTTGAT | TCGTCCCGAGGTGG | |
| | | CGCTCACATTACT | CGCCAGCAGTGGAG | |
| | | TAGGTCTCTGCTT | CGGTCCGGGCACCA | |
| | | GAATGTCACAGAT | CCAAGCGCTTTCCC | |
| | | GTTCTCTTAATAA | GAGACCGTCCTGGC | |
| | | AGAAGAGGCAAG | GCGATGCGTCAAGT | |
| | | AAAAGCCACTTTA | ACACTGAAATTCAT | |
| | | TTATTTATTAAACT | CCTGAGATGAGGTG | |
| | | CCCGCATAGAGTG | GGTTGGCGACTAAG | |
| | | CAGTATTATTACT | GCGCACCGGTGGGC | |
| | | GTGTGCCAGACCC | ACTGCGGGGACAGC | |
| | | TGCTTCAAACACA | AGGGCCCCGCGCGC | |
| | | TTCCATGGACTAT | AGGGAAGCCGCCCG | |
| | | AAAATTGCATCTC | GATCGCCCGGAACC | |
| | | TGAGCAGCTCCTA | GGGCATCTTCCGTG | |
| | | GAGCTGGTAGTAA | GCGGGTCAGCCGAG | |
| | | CAACTTACATTTA | AGCCCGCCGGGTGG | |
| | | CTGGGTGATTACC | TGCTGAGTAGGGAG | |
| | | ATGTGCCAGGTAT | TCCCGGGCTCGGGG | |
| | | TGTGCTAAACACG | CTCCGCGGGCCGCT | |
| | | TTGTAGATATTAA | TTCAGGAGCAGCTG | |
| | | CTCACTTAATCCTC | GCCTTGGCACCGAG | |
| | | GTAACAATCCCAT | CGTGCCCGCGGGAG | |
| | | GAAGTAGGTACTG | GCGGGGGGGGGCGC | |
| | | CTACTATCCCGGC | TGCTCGGTGGCTCT | |
| | | TTTACATCTGAAG | GCTGCGTAGCCGGT | |
| | | TACAGAGAGGTTA | GAACACTTGGCACC | |
| | | AGTAACTTGCCCC | GATGCCCGCCTTCT | |
| | | ATGTCATCCAGCA | GGGCAAGGTGCCCT | |
| | | AGAACTAAATTTG | GAGCCCAGCCCCTC | |
| | | AACCCAGAGCTTA | GCCGGGCTGCAGCC | |
| | | GCCACTGATGCCT | CACCCTCGGCGCGC | |
| | | CTTGAGAGAAGGA | TCAGCCCGCTTCAC | |
| | | GTCAGACTTAAGT | CGCTTCAGGGACGG | |
| | | TGAGTCTTTAAAG | AATAGAACTCGCAG | |
| | | GTGGTTGACCAGG | ATGCAGGGTGTCGC | |
| | | CATTTGTCAGAGT | TGACATTTTCAACTT | |
| | | TAAGAAAGAGAG | TTTCTGCGGTTTCCG | |
| | | GTAGGACATCCTT | CCCGCTGTCTCTGA | |
| | | TTCCAGGCAGAGG | CCCGAAAGTGCCCC | |
| | | GCATTGTGTGCAC | CGGACGGTTACAGA | |
| | | ACACGTATAGAAG | GGACACTTAAGTGG | |
| | | CAGGCAGCCCACC | TTTGCAAAGCCTGT | |
| | | CTCATGCTTTCCA | GGTAGGGGAGGAG | |
| | | GGAAGCAAATGTG | GGTGTAGAAGGGCC | |
| | | GCTCAGGTGTAAA | AAACCACGGAACTT | |
| | | GTGCCCGGTTGAT | AGTTTTATTCATTTA | |
| | | GAAGGGAGTTAGC | TATAAAGCAGCACT | |
| | | GGAGGGAGTATAA | CCGATTCTTTTTGCG | |
| | | GGATGTACTGTCT | CGGCCTGAAATGCA | |
| | | GCCCCCTTAGGAC | TGTGACCAGAGAAG | |
| | | ACCTGCAGAGGAT | TAATTAACAAAACA | |
| | | TAAGGTGGCTGTT | ATGTCAACTTCTAA | |
| | | TCTCCCTGGAGGT | AACCGAGACATTAC | |
| | | GGAGTGGGTGGGT | TTAGATGATAAGGC | |
| | | CACTGCACAGGAG | GCAGCAACTCGGTG | |
| | | CCTATAGTTGTTG | AATCTGTACAAACC | |
| | | GTCTTTTAAACTCT | TTGGAAAAAAAACA | |
| | | TATTGGTGTAACC | CATTAGTCTATGGG | |
| | | AGCCACGGAACTC | ACCTTCCAGTTTTCT | |
| | | TGAGGCAAGGGGT | CATGCTCCTTTCCAG | |
| | | TGGGGGTGGGAAG | CTACTAACCTCTCCT | |
| | | GGAAACAGAGAA | AAAGGGAACAACCA | |
| | | AAGGCAAGTGAAA | CTTTTTGGATTTGAT | |
| | | CAGAAGGGGAGGT | TCCCAGGCCTCGCT | |
| | | GCAGTTTCAGAAC | TTCACCGGGAAATT | |
| | | CCAGCCAGCCTCT | ATCGTTGCTTGTAA | |
| | | CTCTTGCTGCCTA | AACAGA | |
| | | GCCTCCTGCCGGC | (SEQ ID NO: 210) | |
| | | CTCATCTTCGCCC | | |
| | | AGCCAACCCCGCC | | |
| | | TGGAGCCCTATGG | | |
| | | CCAACTGCGAGTT | | |
| | | CAGCCCGG | | |
| | | (SEQ ID NO: 209) | | |
| CD33 | CD33 | GAAGGACTTTAGA | CCGGCCACTCCAAA | TCTGC |
| | gRNA | CATGGGGTTCGCA | AACCTGACCTGCTC | AGGGA |
| | exon 3 | TGTCTCAGATGGC | TGTGTCCTGGGCCT | AACAA |
| | | CCTGAAGGTACTG | GTGAGCAGGGAACA | GAGAC |
| | | ATCCAGGCTCTGG | CCCCCGATCTTCTCC | C |
| | | TGCTCCTGGAAGG | TGGTTGTCAGCTGC | (SEQ ID |
| | | CAAGACTCAGATT | CCCCACCTCCCTGG | NO: 226) |
| | | CTGCTCCATCTCCT | GCCCCAGGACTACT | GAGTG |
| | | CCCATCTCTGGGC | CACTCCTCGGTGCT | GCCGG |
| | | GGGTCTCTGGCAT | CATAATCACCCCAC | GTTCTA |
| | | CTCTGGCCCATGA | GGCCCCAGGACCAC | GAGTG |
| | | GGGTCAATCTGTG | GGCACCAACCTGAC | (SEQ ID |
| | | TGGAGGGGACAAG | CTGTCAGGTGAAGT | NO: 241) |
| | | CTCTGAGCATGTG | TCGCTGGAGCTGGT | |
| | | TGGGTCTGAGGTT | GTGACTACGGAGAG | |
| | | CCTCTTCCATGCA | AACCATCCAGCTCA | |
| | | GGGCTGAGGTCTC | ACGTCACCTGTAAG | |
| | | CTGCTCCTCCCCA | TGCTGGGCCAGGAT | |
| | | GCTTCCTGTCCGG | GCTGGGGTCCCTGA | |
| | | CCCTGTAGTCCTTC | GGGTGTAGGGGAGA | |
| | | CCCTCCACTCCCTT | CAGGATGGGCTGGT | |
| | | CCTCTTTTCTGCTC | GCTGGGGACATTTA | |
| | | ACACAGGAAGCCC | GTGTCCTGGAGGCC | |
| | | TGGAAGCTGCTTC | TGGCTGAGTTCGGG | |
| | | CTCAGACATGCCG | AGCCAGAAGGACAT | |
| | | CTGCTGCTACTGC | GAGCCCTGTCCCTT | |
| | | TGCCCCTGCTGTG | CTGCATTTCTGTGGT | |
| | | GGCAGGTGAGTGG | TTCTGGCAGGAGTA | |
| | | CTGTGGGGAGAGG | AGGGGAAATGCCTA | |
| | | GGTTGTCGGGCTG | CCCTTATCTCATCTC | |
| | | GGCCGAGCTGACC | TACCCCCAACTGAA | |
| | | CTCGTTTCCCCAC | GGAAATCCTCTCTT | |
| | | AGGGGCCCTGGCT | CCTCTCCTAGATGTT | |
| | | ATGGATCCAAATT | CCACAGAACCCAAC | |
| | | TCTGGCTGCAAGT | AACTGGTATCTTTCC | |
| | | GCAGGAGTCAGTG | AGGAGATGGCTCAG | |
| | | ACGGTACAGGAGG | GTAGGAAGGAGCCT | |
| | | GTTTGTGCGTCCTC | CCCCGCCTGGGGCT | |
| | | GTGCCCTGCACTT | GTTACTGACATTGA | |
| | | TCTTCCATCCCATA | GTCTGTGTCAGGTTT | |
| | | CCCTACTACGACA | GGTCAGATCTGGAC | |
| | | AGAACTCCCCAGT | TTTCAGAGTCAAAT | |
| | | TCATGGTTACTGG | GTTCAGAGGCAAGG | |
| | | TTCCGGGAAGGAG | CCTGCAGTTAGACA | |
| | | CCATTATATCCAG | CGGGTAGACATCAG | |
| | | GGACTCTCCAGTG | GCACCTTGGAAAAG | |
| | | GCCACAAACAAGC | GATATTTGGGGATG | |
| | | TAGATCAAGAAGT | ACTAGCAACTTCCC | |
| | | ACAGGAGGAGACT | CCTTGCCCATCCAA | |
| | | CAGGGCAGATTCC | ATAATGCTCTTTGTC | |
| | | GCCTCCTTGGGGA | TCCCTCCTGTCTCTG | |
| | | TCCCAGTAGGAAC | AATGTCTTGGGGTA | |
| | | AACTGCTCCCTGA | TTTTATTTTTAATTG | |
| | | GCATCGTAGACGC | ATATGTAATAATAG | |
| | | CAGGAGGAGGGAT | TACATATTTATGGA | |
| | | AATGGTTCATACT | TGGCATAGTGATGT | |
| | | TCTTTCGGATGGA | TTCCATACTAATAA | |
| | | GAGAGGAAGTACC | TGTATAGTAATCAG | |
| | | AAATACAGTTACA | ATCAGGGTAATAGC | |
| | | AATCTCCCCAGCT | ATATCCATCATCTTG | |
| | | CTCTGTGCATGTG | AACATTTATTATTTC | |
| | | ACAGGTGAGGCAC | ATTGTTGTTGGGAA | |
| | | AGGCTTCAGAAGT | CATTCAATATCCCCT | |
| | | GGCCGCAAGGGAA | TTCTAGCTATTTGAA | |
| | | GTTCATGGGTACT | GCTATCTATTATTGT | |
| | | GCAGGGCAGGGCT | TAAGCATAGTCATC | |
| | | GGGATGGGACCCT | CTACAGTGGTATAG | |
| | | GGTACTGGGAGGG | AACACCAGAACTTA | |
| | | GTTTAGGGGTAAA | TTCTTCCTTTCCAGG | |
| | | GCCTGTCGTGCTT | TGTAATCTAGTATC | |
| | | AGCGGGGGAGCTT | CTTTAACAAATCTCT | |
| | | GACCAGAGGTTGA | CTCCTTATCATTGTT | |
| | | TCTTCTCTCAGGCC | CCCCTAACCTTCCC | |
| | | CTCACCTGGACCC | AGCCCTTATTATT | |
| | | TCCCTCCTGATTCT | (SEQ ID NO: 212) | |
| | | GCATCCCCTCTTTC | | |
| | | TCCTCACTAGACT | | |
| | | TGACCCACAGGCC | | |
| | | CAAAATCCTCATC | | |
| | | CCTGGCACTCTAG | | |
| | | AAC | | |
| | | (SEQ ID NO: 211) | | |
| 5′ end of | 5′ end IL2 | TGCTTCATAAGTG | TCCAAAATTGAACA | CGGCA |
| IL2 | gRNA1 | AAGGAGAAATAA | CATAGTTGGAAGTA | CCACA |
| | | AATATAGACAAGT | AAGCACTCCTCAGC | CCACA |
| | | GAACGCTGAAAGA | AAATGTAAAAGAAC | CCGAT |
| | | TTTTGTCACCACC | AGAAAGTACAACAA | (SEQ ID |
| | | AGGCCTGCCCTAC | ACTGTCTCTCAGAC | NO: 227) |
| | | AAGAGCTCCTGAA | CACAGTGCAATCAA | |
| | | GGAAGCGCTAAAC | ACTAAAACTCAGGA | |
| | | ATGGAAAGGAACA | TTAAGAAACTCACT | |
| | | ACCGGTACCAGCC | CAAAACCGCTCAAC | |
| | | ACTGCAAAAACAT | TACATGGAAACTGA | |
| | | GCCAAATTGTAAA | ACAACCTGCTCCTG | |
| | | CACCATTGAGGCC | AATGACTACTGGGT | |
| | | AGGAAGAAACTGC | ACATAACGAAATGA | |
| | | ATCAACTAACGAG | AGGCAGAAGTAAAG | |
| | | CAAAATAACCAGC | ATGTTCTTTGAAAC | |
| | | TAACATCATCATG | CAACGAGAACAAAG | |
| | | ACAGGATCAAATT | ACACAACATACCAG | |
| | | CACACATAACAAT | AATCTCTGGGACAC | |
| | | ATTAACCTTAAAT | ATTCAAAGCAGTGT | |
| | | GTAAATAGGCTAA | GTAGAGGAAAATTT | |
| | | ATGCTCCAATTAA | ATAGCACTAAATGC | |
| | | AAGACACAGACTG | CCACAAGAGAAAGC | |
| | | GCAAACTGGATAA | AGGAAAGATCTAAA | |
| | | AGAGTCAAGACCC | ATTGACAGCCTAAC | |
| | | ATCAGTGTGCTGT | ATCACAATTAAAAG | |
| | | ATTCAGGAAACCC | AACTAGAGAAACAA | |
| | | ATCTCACGTGCAG | GAGCAAACACATTC | |
| | | AGACACACATAGG | AAAAGCTAGCAGAA | |
| | | CTCAAAATAAAGG | GGCAAGAAATAACT | |
| | | GATGGAGGAAGAT | ACAATCAGAGCAGA | |
| | | CTACCAAGCAAAT | ACTGAAGGAGATAG | |
| | | GGAAAACAAAAA | AGACATACAAAAAA | |
| | | AAGGCAGGGGTTG | CCCTTCAAAAAATC | |
| | | CAATCCTAGTCTC | AATGAATCCAGGAG | |
| | | TGATAAAACAGAC | CTGGTTTTTTGAAA | |
| | | TTTAAACCAACAA | AGATCAACAAAATT | |
| | | AGATCAAAAGAGA | GATAGACCACTAGC | |
| | | CACAGAAGGCCAT | AAAACTAATACAGA | |
| | | TACATAATGGTAA | AGAGAGAAGAATCA | |
| | | AGGGATCAATTCA | AATAGACACAATAA | |
| | | ACAAGAAGAGTTA | AAAATGATAAACGG | |
| | | ACTATCCTAAATA | GATATCACCACTGA | |
| | | TATATGCACCCAA | TCCCACAGAAATAC | |
| | | TACAGGAGCACCT | AAACTACCATCAAA | |
| | | AGATTCATAAAGC | GAATACTATAAACA | |
| | | AAGTCCTTAGAGA | CCTCTATGCAAATA | |
| | | CCTACAAAGAGAC | AACTAGAAAATCTA | |
| | | TTAGACTCCCACA | GAAGAAATGGATAA | |
| | | CAATAATAATGGG | ATTCCTCGACACAT | |
| | | AGACTTTAACACC | ACACCCTCCCAAGA | |
| | | CCACTGTCAACAT | CTAAACCAGGAAGA | |
| | | TAGACAGATCAAT | AGCTGAATCTCTGA | |
| | | GAGACAGAAAATT | ATAGACCAATAACA | |
| | | AACAAGGATATCC | GGCTCTGAAATTGA | |
| | | AGGAATTGAACTC | GGCAACAATTAACA | |
| | | AACTCTGCACCAA | CCTTACCAACCAAT | |
| | | GCGGACCTAATAG | AA | |
| | | ACATCTACAGAAC | (SEQ ID NO: 214) | |
| | | TCTCCACCCCAAA | | |
| | | TCAACAGAATATA | | |
| | | CATTCTTTT | | |
| | | (SEQ ID NO: 213) | | |
| 3′ end of | 3′ end of | GGTGCCTGCCACC | GAAACATAAAACTC | ACCTC |
| B2M | B2M | ACGCCAGGTTAAT | CGAGGTACTTCCGG | GTTCC |
| | gRNA1 | TTTTTGTATTTTTA | CTCCCTCCAGTGTCT | ACAAC |
| | | GTAGAGACAGGGT | GAGGGTAATCTGCA | CCTGG |
| | | TTCACCGTGTTAG | GGACTGAGGGTAAT | (SEQ ID |
| | | CCAGGATGGTCTC | TGCAGATGCTAGCT | NO: 228) |
| | | GATCTCCTGACCT | GTTAGCAGCAAACT | |
| | | CATGATCCACCCA | ATGCAAAAGCTGAG | |
| | | TCTTGGCCTCCCA | GACTGGCTTATAAA | |
| | | AAGTGCTGGGATT | GCCAGATCTGGGTG | |
| | | ACAGGCATGAACC | AGTCATGTTTCCTGT | |
| | | ACTGCGCCCGGCC | CAACATCCTCTGCT | |
| | | GCATCGCTAGTTT | GGGCCCATAACACA | |
| | | TTAAAAACTTTTT | TGCAACCCCAAACT | |
| | | GTAGAGACAGATT | TCCATTACAAGTTC | |
| | | CTTACTATGTTGCC | AAAGTTTCTAAGGG | |
| | | AAGGCTGGTCTCA | GATAGCATTACAGT | |
| | | AACTCCTGGCCTC | GTGTATGATATTGG | |
| | | AAGAGATCCTCCA | ACTCAGACCTGAGT | |
| | | GTCTTCGGCCTCC | TTGAATCCTAATTCC | |
| | | CAAAAAGATGGGA | ACAAAAGAAATTGG | |
| | | TTACAGGCATGAG | AAAAGAGTCATATT | |
| | | CCACCTCACCTGG | GCTGACTTGACCCT | |
| | | CCTCTTTTTTTTGT | TTGTCACCATATCC | |
| | | ATATTACCTGATC | ATAAAATGGGATAA | |
| | | TCAGGTATTCTGC | TTATTCCTATATCAT | |
| | | TATAGCAACAGAA | AAATTTACTTATTTA | |
| | | AGACGAAGACAG | TTCACTTAGTCATTT | |
| | | AATCCTTAGCTGT | GTTAAATAAATATG | |
| | | CTGCAAGTGTGCA | GAGTGTCTACTTTGT | |
| | | TGCCATTTTCATCA | GCCGGGCACTCTTT | |
| | | TCTGAAGAGTCAG | TTAGGGTGGTTCTG | |
| | | CGAGTGTCTTAGG | AGAAGGGGATGGCA | |
| | | TGGAGTCTTGCAA | ATGAGAAGGGCTCT | |
| | | AAGCAGGCCCTGA | CTAAGATGCAAGAC | |
| | | GCCAAAGATTTGG | TCCAGGCAACTGCT | |
| | | ATGCAAATGACTT | TTTACTTCCAGTGGT | |
| | | GTTAAGAAAGGGC | TCTTTATTTTCACAG | |
| | | TCTTCGAGACCGT | CTCATTAGAGCAAA | |
| | | GCCATTGCACTCC | TTACCACAGCAGGG | |
| | | AGCCCGGGCAAGA | AGATACAGGTTGAG | |
| | | AGAGTGAAACTCT | TATCCCTTATCCGA | |
| | | GTTTCAAAAAAAA | AAAGCCTGGCACCA | |
| | | AAAAGTGGGGGGC | GAAGTGTTTTAAAT | |
| | | TCCTAGGAAAAGA | TTTGGATTTTTTTTT | |
| | | ACAGTAAAGGAGT | GATTTTTGGAATATT | |
| | | GGGGGATGAAGG | TGTTAATTATCAGTT | |
| | | ACAGGGAATGGGA | GAGCATCTCTAATG | |
| | | AGAAGCCAAGCGA | TGAAAATCTAAAAT | |
| | | GAGCATGATTTCC | CCAAAATGCCCCAG | |
| | | GAAGTCCTACACT | TGACCCTTTCCTTTG | |
| | | CAGCCTGATCACA | AGCATCATGTTGGT | |
| | | CGGGAAGCTTTAG | GCTCAAAAAGTTTG | |
| | | AACAAAGAACACA | AGATGTTGGAGCAT | |
| | | CCTCAGAGTTTTTC | TTAGGATTTCA | |
| | | CTGCCTCAACACA | (SEQ ID NO: 216) | |
| | | AAGGAGCTGGGCT | | |
| | | TTGGTGCTCTTCAT | | |
| | | CAGCCTGTCTTTG | | |
| | | GCTAT | | |
| | | (SEQ ID NO: 215) | | |

### (2) Nucleic Acids Encoding an Inactivated Cell Surface Receptor

In another general aspect, the invention relates to an isolated nucleic acid encoding an inactivated cell surface receptor useful for an invention according to embodiments of the application. It will be appreciated by those skilled in the art that the coding sequence of an inactivated cell surface receptor can be changed (e.g., replaced, deleted, inserted, etc.) without changing the amino acid sequence of the protein. Accordingly, it will be understood by those skilled in the art that nucleic acid sequences encoding an inactivated cell surface receptor of the application can be altered without changing the amino acid sequences of the proteins.

In certain embodiments, an isolated nucleic acid encodes any inactivated cell surface receptor described herein, such as that comprises a monoclonal antibody-specific epitope, and/or a cytokine, such as an IL-15 or IL-2, wherein the monoclonal antibody-specific epitope and the cytokine are optionally operably linked by an autoprotease peptide sequence.

In some embodiments, the isolated nucleic acid encodes an inactivated cell surface receptor comprising an epitope specifically recognized by an antibody, such as ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, avelumab, ofatumumab, panitumumab, or ustekinumab. In some embodiments, the isolated nucleic acid encodes an inactivated cell surface receptor comprising an epitope specifically recognized by cetuximab. In some embodiments, the isolated nucleic acid encodes an inactivated cell surface receptor comprising an epitope specifically recognized by trastuzumab. In some embodiments, the isolated nucleic acid encodes an inactivated cell surface receptor comprising an epitope specifically recognized by bevacizumab. In some embodiments, the isolated nucleic acid encodes an inactivated cell surface receptor comprising an epitope specifically recognized by avelumab. In some embodiments, the isolated nucleic acid encodes an inactivated cell surface receptor comprising an epitope specifically recognized by ipilimumab.

In certain embodiments, the isolated nucleic acid encodes an inactivated cell surface receptor having a truncated epithelial growth factor (tEGFR) variant. Preferably, the inactivated cell surface receptor comprises an epitope specifically recognized by cetuximab, matuzumab, necitumumab or panitumumab, preferably cetuximab.

In certain embodiments, the isolated nucleic acid encodes an inactivated cell surface receptor having one or more epitopes of CD79b, such as an epitope specifically recognized by polatuzumab vedotin.

In certain embodiments, the isolated nucleic acid encodes an inactivated cell surface receptor having one or more epitopes of CD20, such as an epitope specifically recognized by rituximab.

In certain embodiments, the isolated nucleic acid encodes an inactivated cell surface receptor having one or more epitopes of Her 2 receptor, such as an epitope specifically recognized by trastuzumab In certain embodiments, the autoprotease peptide sequence is porcine teschovirus-1 2A (P2A).

In certain embodiments, the truncated epithelial growth factor (tEGFR) variant consists of an amino acid sequence having at least 90%, 91%, 92%, 93%, 94%, 95%, 96%, 97%, 98%, 99% or 100% sequence identity to the amino acid sequence of SEQ ID NO: 71.

In certain embodiments, the monoclonal antibody-specific epitope specifically recognized by polatuzumab vedotin consists of an amino acid sequence at least 90%, such as at least 90%, 91%, 92%, 93%, 94%, 95%, 96%, 97%, 98%, 99% or 100%, identical to SEQ ID NO: 78.

In certain embodiments, the monoclonal antibody-specific epitope specifically recognized by rituximab consists of an amino acid sequence at least 90%, such as at least 90%, 91%, 92%, 93%, 94%, 95%, 96%, 97%, 98%, 99% or 100%, identical to SEQ ID NO: 80.

In certain embodiments, the monoclonal antibody-specific epitope specifically recognized by trastuzumab consists of an amino acid sequence at least 90%, such as at least 90%, 91%, 92%, 93%, 94%, 95%, 96%, 97%, 98%, 99% or 100%, identical to SEQ ID NO: 82.

In certain embodiments, the IL-15 comprises an amino acid sequence having at least 90%, 91%, 92%, 93%, 94%, 95%, 96%, 97%, 98%, 99% or 100% sequence identity to the amino acid sequence of SEQ ID NO: 72.

In certain embodiments, the autoprotease peptide has an amino acid sequence having at least 90%, 91%, 92%, 93%, 94%, 95%, 96%, 97%, 98%, 99% or 100% sequence identity to the amino acid sequence of SEQ ID NO: 73.

In certain embodiments, the polynucleotide sequence encodes a polypeptide comprising an amino acid sequence having at least 90%, 91%, 92%, 93%, 94%, 95%, 96%, 97%, 98%, 99% or 100% sequence identity to the amino acid sequence of SEQ ID NO: 74.

In a particular embodiment, the isolated nucleic acid encoding the inactivated cell surface receptor comprises a polynucleotide sequence at least 90%, such as at least 90%, 91%, 92%, 93%, 94%, 95%, 96%, 97%, 98% or 100%, identical to SEQ ID NO: 75, preferably the polynucleotide sequence of SEQ ID NO: 75.

In certain embodiments, the polynucleotide sequence encodes a polypeptide comprising an amino acid sequence having at least 90%, 91%, 92%, 93%, 94%, 95%, 96%, 97%, 98%, 99% or 100% sequence identity to the amino acid sequence of SEQ ID NO: 79.

In another general aspect, the application provides a vector comprising a polynucleotide sequence encoding an inactivated cell surface receptor useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a inactivated cell surface receptor in the cell. Conventional cloning techniques or artificial gene synthesis can be used to generate a recombinant expression vector according to embodiments of the application.

In a particular aspect, the application provides a vector for targeted integration of an inactivated cell surface receptor useful for an invention according to embodiments of the application. In certain embodiments, the vector comprises an exogenous polynucleotide having, in the 5′ to 3′ order, (a) a promoter; (b) a polynucleotide sequence encoding an inactivated cell surface receptor, such as an inactivated cell surface receptor comprising a truncated epithelial growth factor (tEGFR) variant and an interleukin 15 (IL-15), wherein the tEGFR variant and IL-15 are operably linked by an autoprotease peptide sequence, such as porcine teschovirus-1 2A (P2A), and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EF1a, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to BGH, hGH, and PGK.

In certain embodiments, the polynucleotide sequence encoding an inactivated cell surface receptor comprises the polynucleotide sequence at least 90%, such as at least 90%, 91%, 92%, 93%, 94%, 95%, 96%, 97%, 98% or 100%, identical to SEQ ID NO: 75.

In some embodiments, the vector further comprises a left homology arm and a right homology arm flanking the exogenous polynucleotide.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 84. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 85 In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 86, preferably the polynucleotide sequence of SEQ ID NO: 86.

### (3) Nucleic Acids Encoding an HLA Construct

In another general aspect, the invention relates to an isolated nucleic acid encoding an HLA construct useful for an invention according to embodiments of the application. It will be appreciated by those skilled in the art that the coding sequence of an HLA construct can be changed (e.g., replaced, deleted, inserted, etc.) without changing the amino acid sequence of the protein. Accordingly, it will be understood by those skilled in the art that nucleic acid sequences encoding an HLA construct of the application can be altered without changing the amino acid sequences of the proteins.

In certain embodiments, the isolated nucleic acid encodes an HLA construct comprising a signal peptide, such as an HLA-G signal peptide, operably linked to an HLA coding sequence, such as a coding sequence of a mature B2M, and/or a mature HLA-E. In some embodiments, the HLA coding sequence encodes the HLA-G and B2M, which are operably linked by a 4× GGGGS linker, and/or the B2M and HLA-E, which are operably linked by a 3× GGGGS linker. In a particular embodiment, the isolated nucleic acid encoding the HLA construct comprises a polynucleotide sequence at least 90%, such as at least 90%, 91%, 92%, 93%, 94%, 95%, 96%, 97%, 98% or 100%, identical to SEQ ID NO: 67, preferably the polynucleotide sequence of SEQ ID NO: 67. In another embodiment, the isolated nucleic acid encoding the HLA construct comprises a polynucleotide sequence at least 90%, such as at least 90%, 91%, 92%, 93%, 94%, 95%, 96%, 97%, 98% or 100%, identical to SEQ ID NO: 70, preferably the polynucleotide sequence of SEQ ID NO: 70.

In another general aspect, the application provides a vector comprising a polynucleotide sequence encoding a HLA construct useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a HLA construct in the cell. Conventional cloning techniques or artificial gene synthesis can be used to generate a recombinant expression vector according to embodiments of the application.

In a particular aspect, the application provides vectors for targeted integration of a HLA construct useful for an invention according to embodiments of the application. In certain embodiments, the vector comprises an exogenous polynucleotide having, in the 5′ to 3′ order, (a) a promoter; (b) a polynucleotide sequence encoding an HLA construct; and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EF1a, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to BGH, hGH, and PGK.

In certain embodiments, a polynucleotide sequence encoding a HLA construct comprises a signal peptide, such as a HLA-G signal peptide, a mature B2M, and a mature HLA-E, wherein the HLA-G and B2M are operably linked by a 4× GGGGS linker (SEQ ID NO: 31) and the B2M transgene and HLA-E are operably linked by a 3× GGGGS linker (SEQ ID NO: 25). In particular embodiments, the HLA construct comprises the polynucleotide sequence at least 90%, such as at least 90%, 91%, 92%, 93%, 94%, 95%, 96%, 97%, 98% or 100%, identical to SEQ ID NO: 67, preferably the polynucleotide sequence of SEQ ID NO: 67. In another embodiment, the HLA construct comprises the polynucleotide sequence at least 90%, such as at least 90%, 91%, 92%, 93%, 94%, 95%, 96%, 97%, 98% or 100%, identical to SEQ ID NO: 70, preferably the polynucleotide sequence of SEQ ID NO: 70.

In some embodiments, the vector further comprises a left homology arm and a right homology arm flanking the exogenous polynucleotide.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 87. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 88.

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 89, preferably the polynucleotide sequence of SEQ ID NO: 89.

### (4) Host Cells

In another general aspect, the application provides a host cell comprising a vector of the application and/or an isolated nucleic acid encoding a construct of the application. Any host cell known to those skilled in the art in view of the present disclosure can be used for recombinant expression of exogenous polynucleotides of the application. According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Examples of host cells include, for example, recombinant cells containing a vector or isolated nucleic acid of the application useful for the production of a vector or construct of interest; or an engineered iPSC or derivative cell thereof containing one or more isolated nucleic acids of the application, preferably integrated at one or more chromosomal loci. A host cell of an isolated nucleic acid of the application can also be an immune effector cell, such as a T cell or NK cell, comprising the one or more isolated nucleic acids of the application. The immune effector cell can be obtained by differentiation of an engineered iPSC of the application. Any suitable method in the art can be used for the differentiation in view of the present disclosure. The immune effector cell can also be obtained transfecting an immune effector cell with one or more isolated nucleic acids of the application.

### Compositions

In another general aspect, the application provides a composition comprising an isolated polynucleotide of the application, a host cell and/or an iPSC or derivative cell thereof of the application.

In certain embodiments, the composition further comprises one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD).

In certain embodiments, the composition is a pharmaceutical composition comprising an isolated polynucleotide of the application, a host cell and/or an iPSC or derivative cell thereof of the application and a pharmaceutically acceptable carrier. The term “pharmaceutical composition” as used herein means a product comprising an isolated polynucleotide of the application, an isolated polypeptide of the application, a host cell of the application, and/or an iPSC or derivative cell thereof of the application together with a pharmaceutically acceptable carrier. Polynucleotides, polypeptides, host cells, and/or iPSCs or derivative cells thereof of the application and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition described herein or the biological activity of a composition described herein. According to particular embodiments, in view of the present disclosure, any pharmaceutically acceptable carrier suitable for use in a polynucleotide, polypeptide, host cell, and/or iPSC or derivative cell thereof can be used.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the application.

### Methods of Use

Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

Cancer conditions may be characterized by the abnormal proliferation of malignant cancer cells and may include leukemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreatic cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The tumour antigens that elicit the immune response may be specific to cancer cells or may be shared by one or more normal cells in the individual.

The cancer cells of an individual suitable for treatment as described herein may express the antigen and/or may be of correct HLA type to bind the antigen receptor expressed by the T cells.

In particular, the cancer cells of an individual suitable for treatment as described herein express the antigen Nectin 4. Nectin4 is expressed in high frequency in bladder, breast, lung, pancreatic, ovarian, head & neck, and esophageal cancers. The highest levels of expression of Nectin4 are seen in bladder, breast, lung and pancreatic cancers. Clinical validation of Nectin4 as a tumor target has been demonstrated by the approval of Enfortumab vedotin for the treatment of urothelial cancer.

An individual suitable for treatment as described above may be a mammal. In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment. In some embodiments, the individual may have no minimal residual disease after one or more cancer treatments or repeated dosing.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

An anti-tumor effect is a biological effect which can be manifested by a reduction in the rate of tumor growth, decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies, also T cells which may be obtained according to the methods of the present invention, as described herein in prevention of the occurrence of tumors in the first place.

Treatment may be any treatment and/or therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of T cells, and a decrease in levels of tumor-specific antigens. Administration of T cells modified as described herein may improve the capacity of the individual to resist cancer growth, in particular growth of a cancer already present the subject and/or decrease the propensity for cancer growth in the individual.

This application provides a method of treating a disease or a condition in a subject in need thereof. The methods comprise administering to the subject in need thereof a therapeutically effective amount of cells of the application and/or a composition of the application. In certain embodiments, the disease or condition is cancer. The cancer can, for example, be a solid or a liquid cancer. The cancer, can, for example, be selected from the group consisting of a lung cancer, a gastric cancer, a colon cancer, a liver cancer, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors. In a preferred embodiment, the cancer is a non-Hodgkin's lymphoma (NHL).

According to embodiments of the application, the composition comprises a therapeutically effective amount of an isolated polynucleotide, an isolated polypeptide, a host cell, and/or an iPSC or derivative cell thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to a cell of the application and/or a pharmaceutical composition of the application a therapeutically effective amount means an amount of the cells and/or the pharmaceutical composition that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the application and/or the pharmaceutical compositions of the application can be administered in any convenient manner known to those skilled in the art. For example, the cells of the application can be administered to the subject by aerosol inhalation, injection, ingestion, transfusion, implantation, and/or transplantation. The compositions comprising the cells of the application can be administered transarterially, subcutaneously, intradermally, intratumorally, intranodally, intramedullary, intramuscularly, intrapleurally, by intravenous (i.v.) injection, or intraperitoneally. In certain embodiments, the cells of the application can be administered with or without lymphodepletion of the subject.

The pharmaceutical compositions comprising cells of the application can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the application in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the application.

The cells of the application and/or the pharmaceutical compositions of the application can be administered in any physiologically acceptable vehicle. A cell population comprising cells of the application can comprise a purified population of cells. Those skilled in the art can readily determine the cells in a cell population using various well known methods. The ranges in purity in cell populations comprising genetically modified cells of the application can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the application are generally administered as a dose based on cells per kilogram (cells/kg) of body weight of the subject to which the cells and/or pharmaceutical compositions comprising the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the application are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

The cells of the application and/or the pharmaceutical compositions of the application can be administered in combination with one or more additional therapeutic agents. In certain embodiments the one or more therapeutic agents are selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD). In certain embodiments, the one or more therapeutic agents comprise an antibody. In certain embodiments, the one or more therapeutic agents comprise one or more antibodies independently selected from the group consisting of ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, avelumab, ofatumumab, panitumumab, and ustekinumab. In certain embodiments, the one or more therapeutic agents comprise cetuximab. In certain embodiments, the one or more therapeutic agents comprise trastuzumab. In certain embodiments, the one or more therapeutic agents comprise bevacizumab. In certain embodiments, the one or more therapeutic agents comprise avelumab. In certain embodiments, the one or more therapeutic agents comprise ipilimumab.

### EXAMPLES

Abbreviations

| | | | |
|---|---|---|---|
| BSA | Bovine Serum Albumin | VHH | Single variable domain on a |
| CAR | Chimeric Antigen Receptor | | heavy chain |
| CD | Cluster Of Differentiation | KO | Knockout |
| E:T | Effector To Target Ratio | ug | Microgram |
| Fc | Fragment Crystallizable | ° C. | Degrees Celsius |
| Ig | Immunoglobulin | dH2O | Distilled water |
| M | Molar | L | Liter |
| uM | Micromolar | mL | Milliliter |
| nM | Nanomolar | uL | Microliter |
| UTD | Untransduced | hrs | Hours |
| | | rpm | Rounds per minute |
| × g | Times gravity | RT | Room temperature |
| OD | Optical density | kV | Kilovolts |
| Min | Minutes | μF | Microfarads |
| PPE | Periplasmic Extract | | |

### Example 1. Preparation of Phagemid Libraries

Objective: Clone VHH Library into Phagemid at Scale to Generate Glycerol Stocks for Later Phage Production

### Materials:

Cells: MC1061F′ (Lucigen #60512-2)
Buffers:
   D-PBS (Dulbecco's Phosphate Buffered Saline; Life Technologies #14190)
   TBST (Tris-buffered saline with 0.05% Tween-20; Sigma #79039-10PAK)
   PBST (D-PBS with 0.05% Tween-20)
   Tween-20 (Sigma #P-7949)
   PBST-M (PBST with 3% non-fat dry milk)
   2×YT (Teknova #Y0167)
   LB (Carb) agar plates (Teknova #L1010)
   Carbenicillin aka “Carb” (Novagen #69101)—concentration=100 mg/mL
   Kanamycin aka “Kan” (Sigma #60615)—concentration=35 mg/mL
   Tetracycline aka “Tet” (Sigma #T3383-25 g)—concentration=15 mg/mL
   IPTG (Isopropyl β-D-1-thiogalactopyranoside; Sigma #69101)—concentration=1 M
   PEG/NaCl (Teknova #P4138)
   M13K07 (Antibody Design Labs #NC1591729)
Enzymes:
   NcoI (NEB #R3193L)
   XhoI (NEB #R0146L)
   T4 DNA Ligase (Invitrogen #15224090)
Kits:
   PCR clean up kit (Qiagen, #28104)
   QIAquick Gel extraction kit (Qiagen, #28704)
Oligonucleotides:

-PelB_F
ATGAAATACCTATTGCCTACGGCAGCC
-P3_Spe_R
CTCAGAACCGCCACCTTCACTAGT

### Procedure:

VHH V2-Medium library were PCR amplified with PelB_F+P3_Spe_R oligonucleotides for 13 cycles at 60° C. annealing temperature. The VHH library fragment was gel isolated and digested with NcoI+XhoI restriction enzymes for 1.5 hours at 37° C. The digested DNA was subsequently column purified. Similarly, the V2-Short library (which was previously amplified DNA) was digested with NcoI+XhoI restriction enzymes for 1.5 hours at 37° C. The digested DNA was subsequently column purified. 15 ug of P222 vector was also digested with NcoI+XhoI for 1.5 hrs at 37° C., and isolated by gel isolation. Ligation was performed by combining 4 μg of the P222 vector with 1.376 ug of VHH V2 short or medium fragments at room temperature for 6 hours, and subsequently overnight at 18° C. Ligation products were column purified using Qiaquick MinElute PCR purification kit into 22 uL dH20.

### Library Transformations

500 uL MC1061F′ cells were added to each ligation. Approximately 26 uL was aliquoted to each of 20 1 mm gap BTX electroporation cuvettes. Each aliquot was electroporated & re-suspended in 1 mL recovery media, and subsequently transferred to a 125 mL shake flask. Each cuvette was then filled with 1 mL media & transferred to a 125 mL shake flask, for a total volume of approximately 40 mL. The cells were then grown by placing the flask at 37° C. for 1 hour. 10 μL of the cell culture was diluted into 90 uL media, and the dilution was repeated 6 times. Dilutions 4, 5 & 6 were plated on LB (Carb) agar plates and incubated at 37° C. overnight.
V2-short: 4.0×10\hat{ }9 transformants
V2-medium: 1.95×10\hat{ }9 transformants

| | Dilution | Colonies | Total | Extended |
|---|---|---|---|---|
| V2M 1 | 0.000001 | 35 | 3.50E+07 | 7.00E+08 |
| V2M 20 | 0.000001 | 65 | 6.50E+07 | 1.95E+09 |
| V2S 1 | 0.000001 | 63 | 6.30E+07 | 1.32E+09 |
| V2S 20 | 0.000001 | 115 | 1.15E+08 | 4.03E+09 |

1-1 uL of ligated DNA in test transformation
20—Full amount of ligated DNA into 20 cuvettes of 25 uL
MC1061F′ cells
Extended value=total number of transformants

The cell culture was grown in 1 L 2×YT (Carb) at 37° C., and the OD600 was monitored hourly. When the OD600 equaled 1, the sample was harvested by centrifugation at 4800×G, and resuspended in 50 mL 2×YT (Carb, 20% glycerol). The sample was aliquoted into 5×10 mL volumes in 15 mL conical tubes and stored at −80 Celsius.

### Example 2. Generation of Phage from VHH Phagemid Libraries

### Materials:

Cells:
   MC1061F′ (Lucigen #60512-2)
Buffers:
   D-PBS (Dulbecco's Phosphate Buffered Saline; Life Technologies #14190)
   TBST (Tris-buffered saline with 0.05% Tween-20; Sigma #79039-10PAK)
   PBST (D-PBS with 0.05% Tween-20)
   Tween-20 (Sigma #P-7949)
   PBST-M (PBST with 3% non-fat dry milk)
   2×YT (Teknova #Y0167)
   LB (Carb) agar plates (Teknova #L1010)
   Carbenicillin aka “Carb” (Novagen #69101)—concentration=100 mg/mL
   Kanamycin aka “Kan” (Sigma #60615)—concentration=35 mg/mL
   Tetracycline aka “Tet” (Sigma #T3383-25 g)—concentration=15 mg/mL
   IPTG (Isopropyl β-D-1-thiogalactopyranoside; Sigma #69101)—concentration=1 M
   PEG/NaCl (Teknova #P4138)
   M13K07 (Antibody Design Labs #PH010L)

### Procedure:

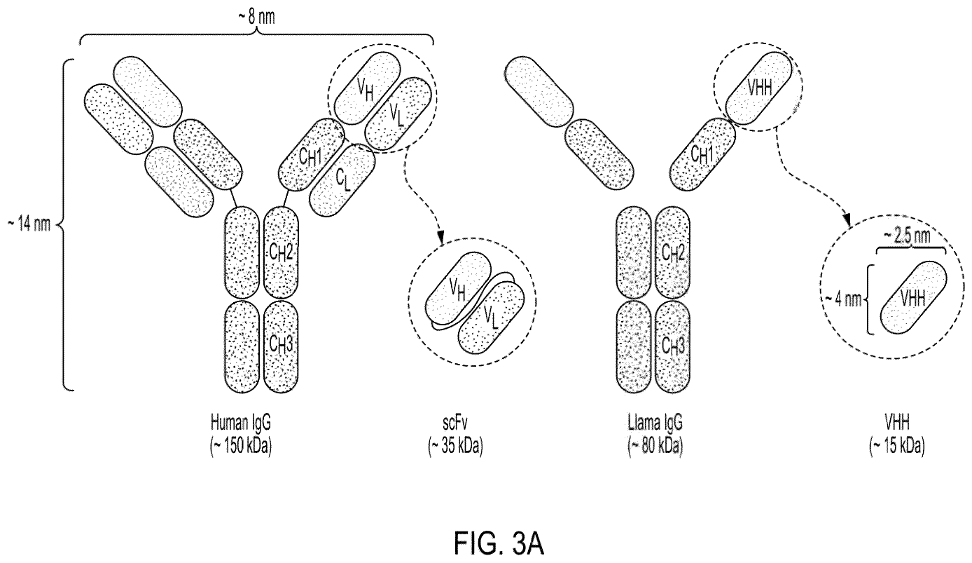

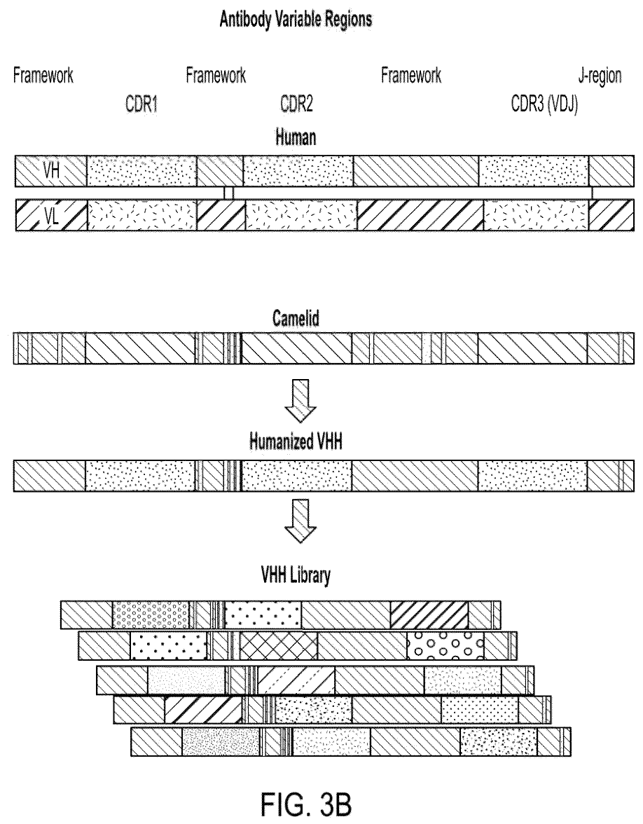

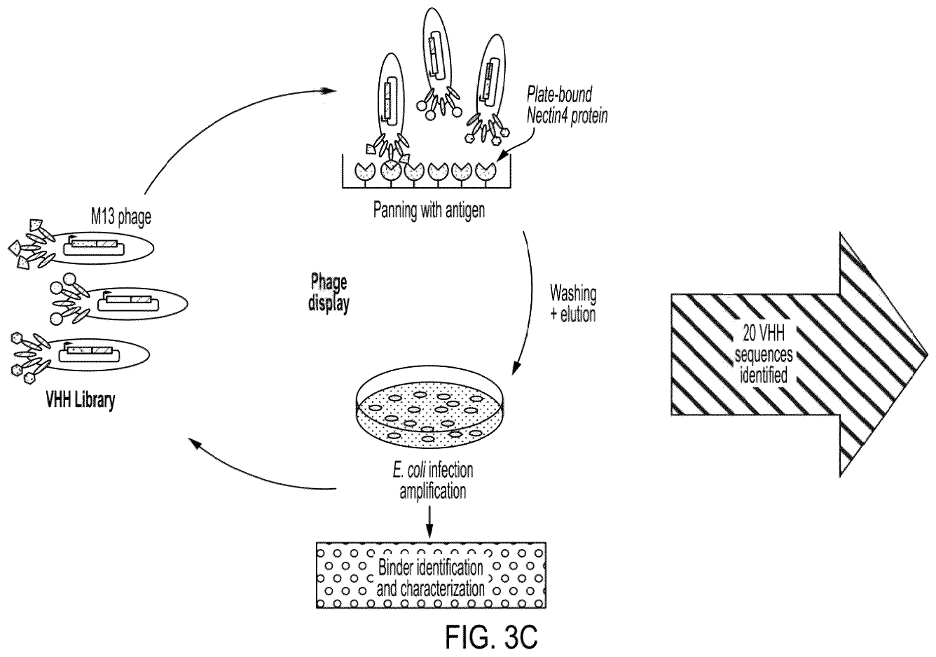

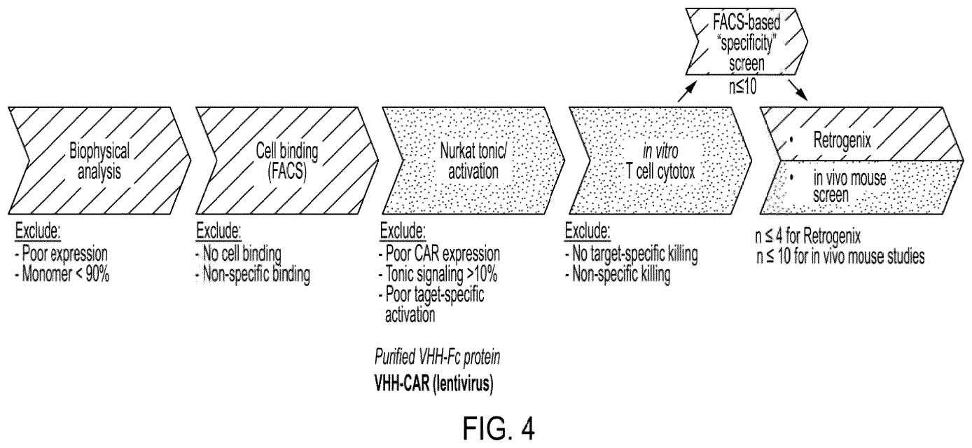

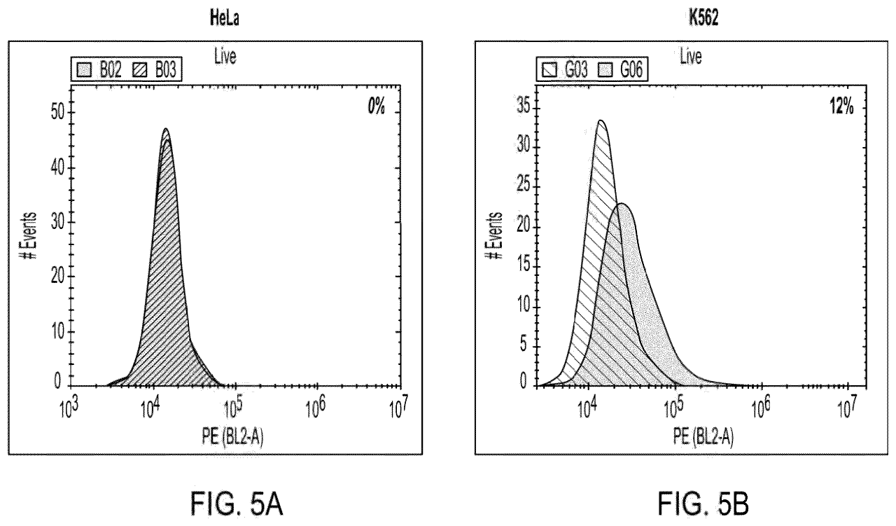

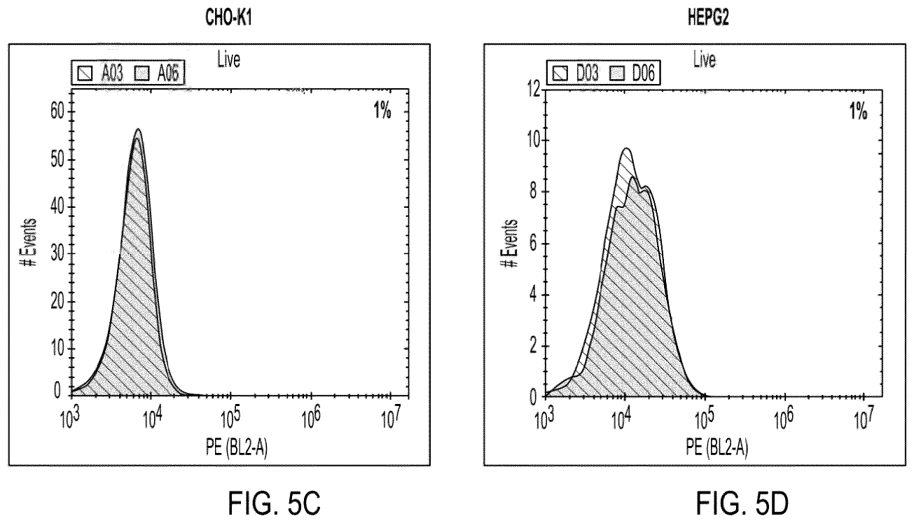

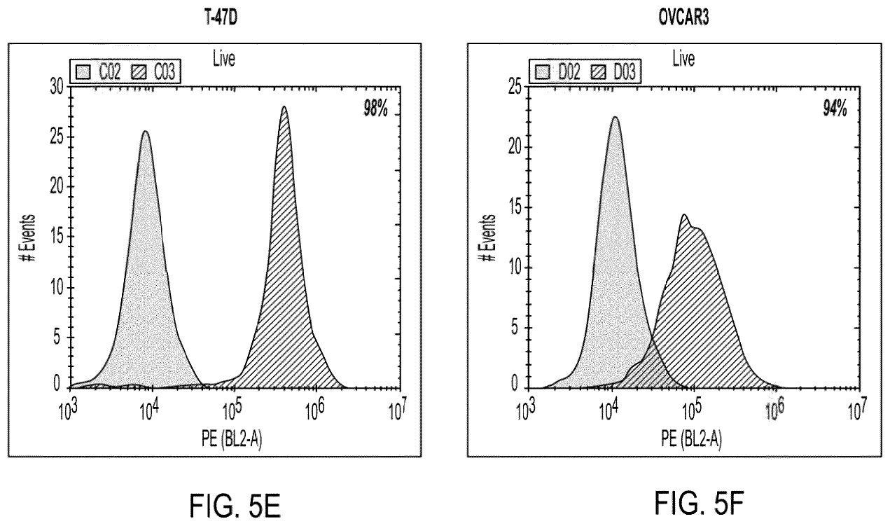

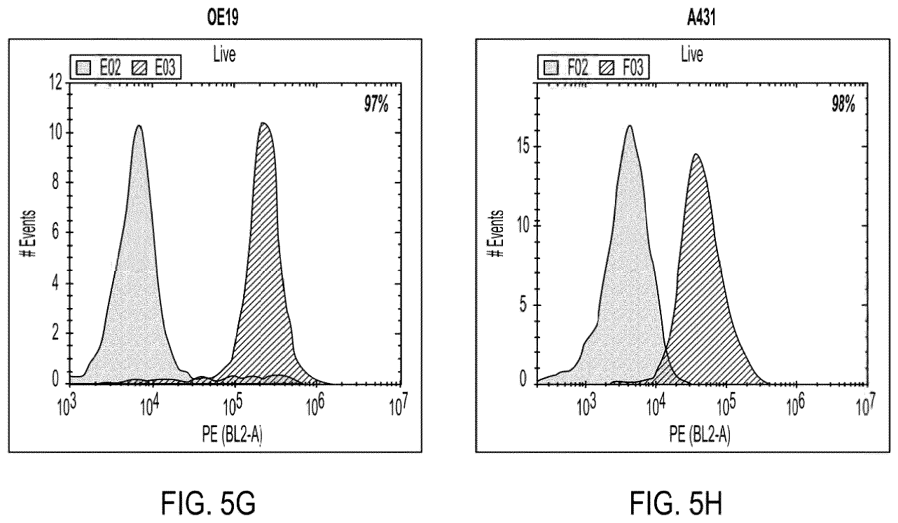

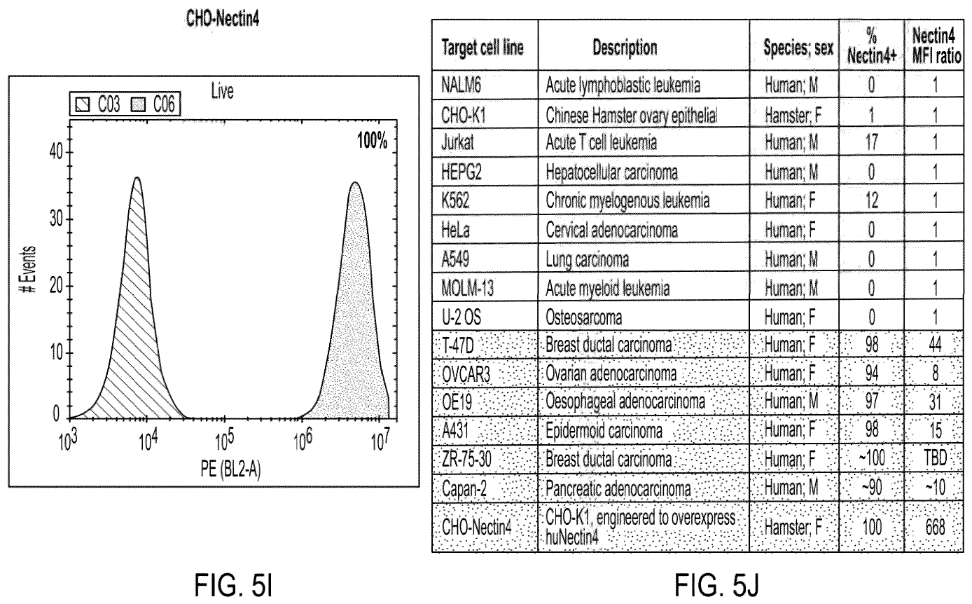

Helper Phage Infection: VHH phagemid libraries were prepared as shown in FIG. 3B. 10 mL of library glycerol stock was thawed for each library (Short & Medium). Glycerol stock was diluted into 990 mL 2×YT (Carb) in 2 1 L flasks. The samples were grown at 37° C. shaking at 225 rpm until OD equaled 0.6. Add 1 mL M13K07 to the culture mix for 30 minutes at 37° C. 1 mL of Kanamycin and 1 mL or IPTG were added, and the mixture was incubated overnight at 30° C. at 225 rpm.

Phage Harvest: Cells from overnight culture were centrifuged at 4400×g for 15 minutes at 4° C. The supernatant was transferred into fresh tubes, and the centrifugation was repeated to remove all cells. The supernatant was transferred to a 1 L bottle, and 100 mL PEG/NaCl was added. The mixture was placed on ice for 1 hour, transferred to 50 mL centrifuge tubes, and the phage was centrifuged at 13000×g for 10 minutes at 4° C. The supernatant was discarded, and the phage was resuspended in 100 mL of D-PBS. The sample was centrifuged at 8000×g for 5 minutes at 4° C., and the supernatant was harvested and aliquoted into cryovials and frozen at −80° C.

Titer: A titer was performed on MC1061F′ cells using 10 μL of remaining phage. MC1061F′ cells were grown in 45 mL 2×YT (Tet) until OD equaled 0.5. 10 uL of phage was diluted in 1:10 serial dilutions across a plate (12 total dilutions). 10 uL of each dilution was added to 90 uL MC1061F′ cells and incubated at 37° C. for 30 min. Each infected well was spotted onto LB (Carb) agar plates, and the full well volume from dilutions 8-10 were plated onto separate plates and incubated at 37° C. overnight. Also, the 8th and 9th dilution of MC1061 infected cells were plated on LB (Carb) agar plates. All libraries were titered to the 9th dilution. Plate counts were as follows:

V2S Dilution 9:96 colonies→1.9×10\hat{ }12 pfu/mL

V2M Dilution 9:124 colonies→2.4×10\hat{ }12 pfu/mL

### Example 3. Phage Panning & Screening Against Nectin4 Protein

As shown in FIG. 3C, three rounds of phage panning on Century's VHH Phage library were performed on plate-bound Nectin4-HIS protein (AcroBiosystems #NE4-H52H3) and individual colonies were screened by ELISA using periplasmic extract (PPE).

### Phage Panning

### Materials:

Libraries (Prepared according to Examples 1 & 2):
   VHH-V2-Short
   VHH-V2-Medium
Cells:
   MC1061F′ (Lucigen #60512-2)
Buffers
   D-PBS (Dulbecco's Phosphate Buffered Saline; Life Technologies #14190)
   TBST (Tris-buffered saline with 0.05% Tween-20; Sigma #79039-10PAK)
   PBST (D-PBS with 0.05% Tween-20)
   Tween-20 (Sigma #P-7949)
   PBST-M (PBST with 5% non-fat dry milk)
   2×YT (Teknova #Y0167)
   LB (Carb) agar plates (Teknova #L1010)
   Carbenicillin aka “Carb” (Novagen #69101)—concentration=100 mg/mL
   Kanamycin aka “Kan” (Sigma #60615)—concentration=35 mg/mL
   Tetracycline aka “Tet” (Sigma #T3383-25 g)—concentration=15 mg/mL
   IPTG (Isopropyl β-D-1-thiogalactopyranoside; Sigma #69101)—concentration=1 M
   PEG/NaCl (Teknova #P4138)
   M13K07 (NEB #N0315S)
Enzymes
   NheI (NEB #R0131 M)
   SpeI (NEB #R0133 M)
   T4 DNA Ligase (Invitrogen #15224090)
Kits:
   PCR clean up kit (Qiagen, #28104)
   QIAquick Gel extraction kit (Qiagen, #28704)
   QIAprep Spin Miniprep Kit (Qiagen #27106)
Proteins
   Nectin4 (AcroBiosystems #NE4-H52H3)
   Streptavidin (VWR #VWRVE497-5 MG)
   Human IgG (whole molecule; Jackson ImmunoResearch #009-000-003)

### Methods:

Phage selection for Nectin4 VHH-phage: On day 0, 4 wells of a maxisorp plate were coated with Nectin4 at Sug/mL (200 uL/well) and incubated at 4° C. overnight. On day 1, for the first round of panning, the MC1061F′ culture was started using 0.5 mL glycerol stock in 25 ml 2×YT+25 uL Tet in a 250 ml flask and incubated at 37° C. until OD600 equaled 0.6 (approximately 2.5 hours). 4 Eppendorf tubes were blocked with 400 uL per well with PBST-M. Nectin4 was removed from the wells of the maxisorp plate. The Nectin4-coated wells were blocked with 300 uL PBST-M.

For library blocking, 200 μL of each library was added to 200 uL PBST-M in blocked tubes with 10 μg/mL human IgG (whole molecule) and incubated for 45 minutes at room temperature. To bind antigen, PBST-M was dumped from the maxisorp plate, and 200 μL of blocked library was added per Nectin4-coated well and incubated for 45 min at RT. Washes were performed by dumping the phage mix from the maxisorp plate, and washing each well 6 times with PBS-T and 1 time with PBS. For phase infection and growth, 200 uL MC1061F′ cells (OD600=0.7) was added to each library well of the maxisorp plate and incubated for 30 minutes at 37° C. 10 uL was removed for 6 serial 1:10 dilutions and 2 uL was spotted on LB (carb/glucose) plates to determine output titer, and incubated at 37° C. overnight. A 5th dilution was plated for single colony sequencing. The remaining MC1061F′ cells were withdrawn and grown in 10 mL 2×YT (Carb) until OD equaled 0.6. Each culture was infected with 10 uL of helper phage (NEB) for 30 min. at 37° C. 10 uL IPTG and Kanamycin was added, and the culture was grown overnight at 30° C. 4 wells of a maxisorp plate were coated with Nectin4 at 2 ug/mL (200 uL/well) and incubated at 4° C. overnight.

On day 2, for the second round of panning, the overnight phage cultures from the first round of panning were centrifuged at 6000×g for 10 min. Phage was precipitated with 0.2 volumes of PEG/NaCl for 30 min. on ice. The phage was then centrifuged at 13000×g for 10 min, and resuspended in 1:10 volume of PBS (this is Round 2 input phage). Round 2 panning was completed following the same steps as describes for Round 1 panning.

On day 3, the steps from day 2 were repeated. After elution of phage with MC1061F′ cells, the cells were grown overnight at 37° C. for DNA preparation and pIII excision.

On day 4, each library panning plasmid DNA was miniprepped (Qiagen Plasmid Miniprep Kit). 10 μL of miniprep DNA was digest with NheI+SpeI restriction enzymes at 37° C. for 1 hour.

| Reagent | Volume | |
|---|---|---|
| Miniprep DNA | 10 | uL |
| NheI | 0.5 | uL |
| SpeI | 0.5 | uL |
| NEB Buffer #3 | 2 | uL |
| dH2O | 7 | uL |

The sample was run on a 1% agarose gel & the vector band at 5 kb was gel extracted. DNA was purified with the Qiagen gel extraction kit into 30 uL EB. 3 μL of isolated DNA was ligated for 1 hour with T4 DNA ligase.

| Reagent | Volume | |
|---|---|---|
| Digested DNA | 3 | uL |
| T4 DNA ligase Buffer | 3 | uL |
| T4 DNA ligase | 1 | uL |
| dH2O | 8 | uL |
| Total | 15 | uL |

Ligated DNA was column purified into 30 uL dH2O (Qiagen PCR purification kit). MC1061F′ cells were electroporated with 3 μL of ligated DNA in 1 mm electroporation cuvette at 1.8 kV, 200 ohms, 50 μF. The cells were rescued by adding 950 uL SOC medium at room temperature and diluted 1:10 in 90 uL SOC (3 times). Dilutions 2 & 3 were plated onto LB (Carb) agar plates & incubated overnight at 37° C.

### Screening

### Materials:

Cells:
   MC1061F′ (Lucigen #60512-2)
Enzymes & Kits:
   XhoI (NEB #R0416L)
   NEBuilder HiFi Assembly Master Mix (E2621L)
   NEB 5-alpha cells (NEB #C2987H)
   Q5 2× Master mix (NEB #M0492L)
   OneShot Stable3 cells (Life Technologies #C7373-03)
   Qiagen Gel Extraction kit (Qiagen #28706)
   Qiaquick miniprep kit (Qiagen #27106)
   Qiagen PCR purification kit (Qiagen #28106)
Buffers
   D-PBS (Dulbecco's Phosphate Buffered Saline; Life Technologies #14190)
   TBST (Tris-buffered saline with 0.05% Tween-20; Sigma #79039-10PAK)
   PBST (D-PBS with 0.05% Tween-20)
   Tween-20 (Sigma #P-7949)
   PBST-M (PBST with 3% non-fat dry milk)
   PPB (Teknova 101320-468)
   2×YT (Teknova #Y0167)
   LB (Carb/20% glucose) agar plates (Teknova #L1801)
   Carbenicillin aka “Carb” (Novagen #69101)—concentration=100 mg/mL
   IPTG (Isopropyl β-D-1-thiogalactopyranoside; Sigma #69101)—concentration=1 M
   PEG/NaCl (Teknova #P4138)
Antibodies & detection reagents
   anti-Flag:HRP (Sigma Aldrich #F1804)
   Nectin4 (AcroBiosystems #NE4-H52H3)
   Ultra TMB (ThermoFisher #34028)
   Stop solution (ThermoFisher #N600)
Oligonucleotide Sequences

-P268VHH_F
GCTTAGCGGAGCCAGATGCGAGGTACAACTTTTGGAGTCAGGC
-P268VHH_R
CGGACCGTATTTGGACTCGCTCGAGACCGTCACCTGGGT
-MARS_VHH_F
CTTTCAGGCGCGCGCTGTGAGGTACAACTTTTGGAGTCAGGC
-IgG1_VHH_R
GTCACAACTCTTAGGTTCGCTCGAGACCGTCACCTGGGT

### Procedure:

Periplasmic Protein Preparation: Bacteria from the phase panning was grown in 96-deep well plates. Each colony was grown in 1 mL 2×YT (Carb) at 37° C., shaking, until turbid. 5 uL was spotted onto LB (Carb) rectangular agar plates and incubated at 30° C. overnight. 100 uL 2×YT (Carb) with 1 uL IPTG/mL final concentration was added & the culture grown overnight shaking at 30° C. The cells were harvested by centrifugation at 4,800×g for 10 minutes. The pellet was resuspended in 100 uL PBP, and the cells kept on ice for 20 min. The cell suspension was centrifuged at 4800×g for 10 min. at 4° C. All the supernatant was removed, and the pellet was resuspended in 100 uL ice-cold 5 mM MgSO4. The mixture was incubated for 20 minutes on ice with occasional shaking, and then centrifuged at 4800×g for 10 minutes at 4° C.

ELISA: 8 Maxisorp plates were coated with 100 uL/well Nectin4 at 1 ug/mL overnight at 4° C. Plates were emptied, and 200 uL/well SuperBlock was added to all plates, and incubated for 1 hour at RT. The plate was washed 1× with TBST on the Aquamax plate washer. 60 μL of anti-Flag:HRP (1:10,000 in PBS-T with 1:10 Superblock) was added per well to a V-bottom plate for each library screening plate. 60 uL/well PPE was added per well to the V-bottom plates and incubated for 1 hour at RT. 50 μL of the PPE/Anti-Flag mix was transferred to each ELISA plate and incubated for 1 hour at RT. The plate was washed 3× with TBST on the Aquamax plate washer. 50 uL/well TMB Ultra solution was added and incubated at RT for 30 minutes, until the wells appeared blue. 50 uL/well stop solution was added and absorbance at 450 nm was read on the Spectramax i3× plate reader. 54 unique clones were identified from the two libraries. A subset of 20 clones was selected from V2S and V2 M for cloning into P201 (SEQ ID Nos. 111-130).

### Example 4. VHH-Fc Demonstrate Specific Binding to CHO-Nectin4 Cells

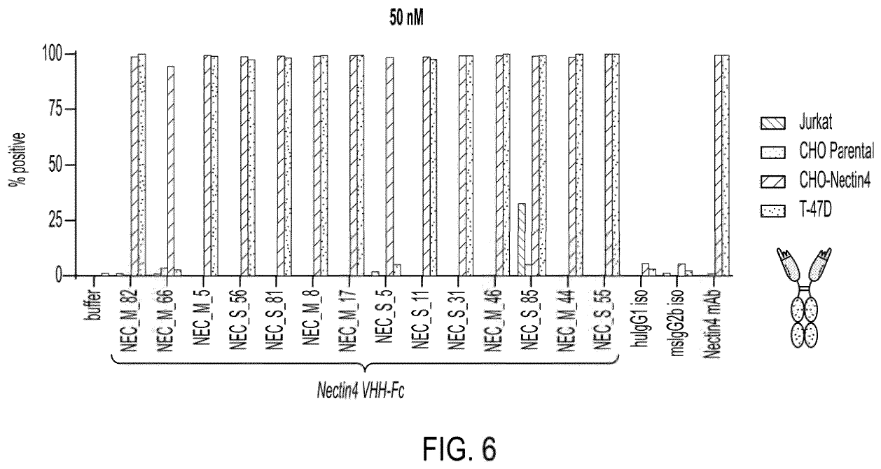

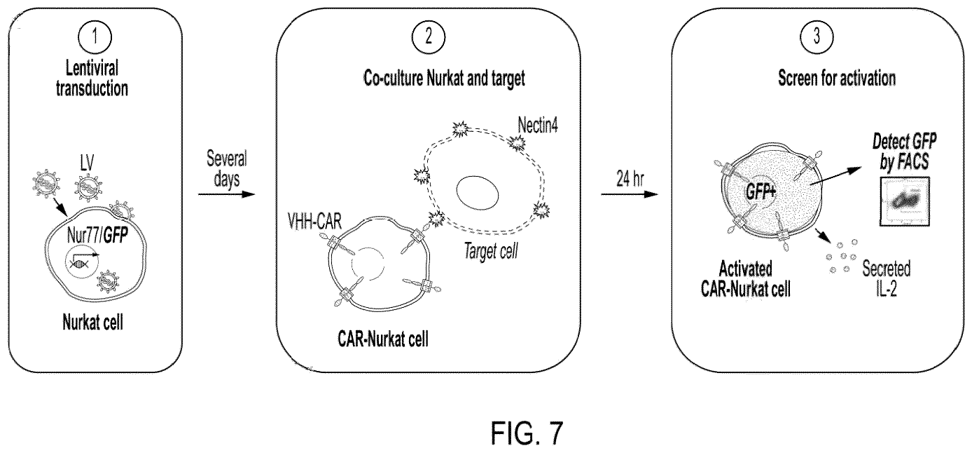

As shown in FIG. 6, 14 anti-Nectin4 VHH-Fc were screened for binding to Nectin4 positive cell lines. All 14 VHH-Fc demonstrated specific binding to CHO-Nectin4 cells, and 12 of 14 cell lines demonstrated specific binding to the Nectin4 positive tumor cell line T47D.

### Materials:

BD Staining Buffer, BSA (BD Pharmingen, Cat #554657)
EDTA, 0.5 M (Corning, Cat #46-034-CI)
Pluronic™ F-68 Non-ionic Surfactant (100×)(Gibco, Cat #24040032)
LIVE/DEAD™ Fixable Near-IR Dead Cell Stain (Thermo, Cat #L10119; 1:1000 dilution)
PE goat anti-hu Fc (JIR, Cat #109-116-098 stock 1 mg/ml, polyclonal, 1 ug/ml)
Innovex Fc Receptor Blocker (Innovex Biosciences, Cat #NB309)
hu IgG1,k isotype (Biolegend, Cat #403502, Clone:QA16A12, concentration 1 mg/ml, 6.67 uM)
Staining Buffer: BD Staining Buffer+2 mM EDTA (Add 2 ml EDTA to 500 ml bottle of BD Staining Buffer)
Running Buffer: BD Staining Buffer+1 mM EDTA+0.1% Pluronic Acid (Add 1 ml EDTA and 5 ml Pluronic Acid to 500 ml bottle of BD Staining Buffer)

### Procedure:

200 ul of cell suspension was collected and counted on VI-cell

| | Cell density | Viability |
|---|---|---|
| Jurkat | 1.59E+06 | 94% |
| CHO Parental | 2.35E+06 | 97% |
| CHO-Nectin4 | 2.07E+06 | 96% |
| T47D | 7.70E+05 | 89% |

50 thousand cells were transferred to v-bottom PP 96-well plate (to perform staining). 150 ul FACS buffer was added to serve as wash. Plate was centrifuged at 300×g for 3 minutes at 4-8° C. Supernatant was aspirated, and plate was gently vortexed to disperse cell pellet. 50 ul Innovex Fc blocker was added, and the plate was incubated at RT for 15-20 minutes. Plate was centrifuged at 300×g for 3 minutes at 4-8° C. Supernatant was aspirated, and plate was gently vortexed to disperse cell pellet. Cells were resuspended in staining condition (50 nM=7.5 ug/ml for mAb; 50 nM=4 μg/ml for VHH-Fc). Plate was incubated at 4° C. for 30 minutes and protected from light exposure. 150 ul Staining buffer to wells (to serve as wash step). Plate was centrifuged at 300×g for 3 minutes at 4-8° C. Supernatant was aspirated, and cells were resuspended in 50 ul staining solution (NearIR+PE anti-hu,IgG,Fc, 1 ug/ml; or Only NearIR for PE mAb controls). Plate was incubated at 4° C. for 30 minutes, and protected from light exposure. 150 ul staining buffer to wells (to serve as wash step), and the plate was centrifuged at 300×g for 3 minutes at 4-8° C. Supernatant was aspirated, and 200 ul staining buffer to wells (to serve as wash step). Plate was centrifuged at 300×g for 3 minutes at 4-8° C., and vortexed to disperse cell pellet. Cells were resuspended in 35 ul staining buffer and analyzed using an IntelliCyt iQue machine.

### Example 5. Jurkat_Nur77 Reporter Assay for Tonic Signaling and Activation Via Nectin4-Positive Cell Lines

Functional activity of anti-Nectin4 VHH-CAR were evaluated using a Nurkat activation assay. Anti-Nectin4 VHH binders were identified, as described herein using phage libraries, and 9 VHH were selected for CAR formatting, based on robust VHH-Fc binding to CHO-Nectin4 and T47D cell lines (see, e.g., Example 4 & FIG. 6). Nur77-Jurkat reporter line (Nurkat) was transduced with CAR lentivirus and GFP expression was evaluated in relation to CAR expression via flow cytometry. In this reporter line, GFP expression is linked to Nur77. Accordingly, background GFP expression corresponds to tonic signaling. Transduced Nurkat cells were screened for activation following co-culture with target cells.

Cells were transduced on day 0. Cells were counted on Vi-cell at 0.66×10⁶ cells/ml at 98% viability. Cells were centrifuged at 300×g for 5 minutes to pellet the Nur77-Jurkat cells, resuspended at 0.45×10⁶ cells/ml in R10 media containing 3 μg/ml polybrene (Boston BioProducts, 10 mg/ml), and plated at 200,000 cells total per well of a 24-well plate. 15 ul of lentivirus was added, and the plate centrifuged at 1300×g at 32° C. for 45 min. Fresh 1 ml R10 media was added for a final volume ~1.5 ml, and incubated at 37° C.

The activation assay was set up on day 3. 50×10³ cells each were stained for GFP and CAR expression (ProteinA, anti-VHH, MSLN protein). Cells were also stained for Nectin4-HIS protein (Acro Biosystems #NE4-H52H3, Lot 2823a-214VF1-WR; stock at 0.4 mg/ml) at a 2 ug/ml concentration. FACS staining was performed on activated CAR-Jurkat cells for CD3 and GFP.

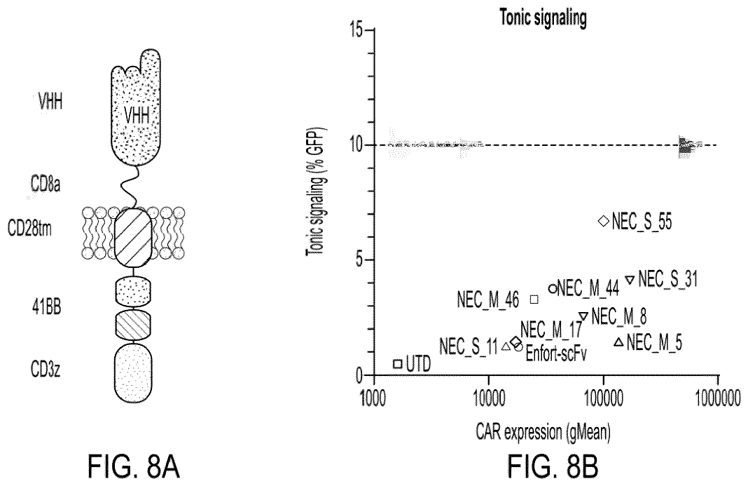

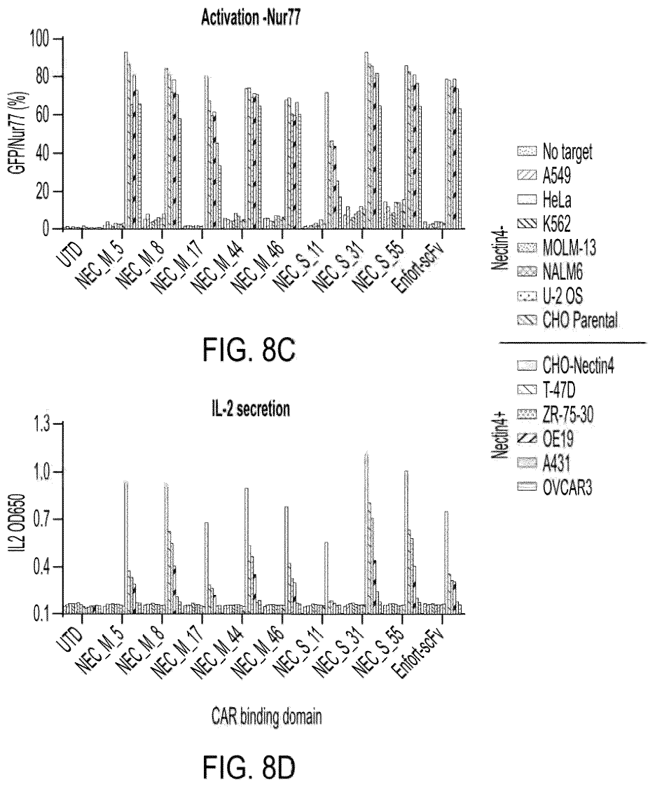

The results of tonic signaling, activation, and IL-2 secretion are provided in FIGS. 8B-D, respectively. CAR expression was confirmed for each construct, ranging from ~60-100% positive CAR expression. ~6/8 Nectin4 VHH-CAR and positive control CARs bound to recombinant Nectin4-HIS protein, and binding was proportional to VHH expression (Geomean). Constructs P3108 and P3112 showed minimal binding to Nectin4 protein, and CAR expression is also lower. All Nectin4 VHH-CAR and positive control CARs demonstrated target-specific activation against CHO-Nectin4 and all Nectin4+ tumor cell lines. Most Nectin4 VHH-CAR demonstrate CAR-mediated activation was comparable to positive control scFv, Enfortumab. Reduced activation was observed against OVCAR3, likely due to lower Nectin4 expression. P3106 (NEC_M_5) exhibited high CAR expression, low tonic signaling (~2%), and robust activation across all Nectin4+ lines.

### Example 6. VHH-CAR T-cells Demonstrate Target-specific Killing of Nectin4+ Cell Lines In Vitro

Primary T cells were transduced with anti-Nectin4 VHH-CAR lentivirus binders, including 8 VHH, and 2 scFv positive controls (P2025 and P2026; Enfortumab in both orientations). CAR expression was confirmed via flow cytometry, and transduced CAR-T were screened using a FACS-based cytotoxicity assay against Nectin4+ target cell lines.

Activated T cells were transduced on Day 0. Cells were collected and counted on Vi-cell: 0.7 E6c/ml @86% viability (6.1 ml total). Cells were pelleted by centrifugation 5 min, at 300×g, to remove TransAct. T cells were resuspended to 0.6 E6c/ml in media. 250 ul cells/well were plated directly into 24-well plate (150,000 T cells/well), and incubated while preparing Transdux mastermix. Transdux Max mastermix was prepared in 50 ml conical tube using TransDux™ MAX Lentivirus Transduction Reagent (SBI, Cat #LV860A-1) and HEPES (Gibco, Cat #15630-080). 250 ul of TransduxMAX mastermix was added to each well of 24 well plate. 60 ul of lentivirus was added directly to the wells. No lentivirus was added to the Mock Transduction (Untransduced; UTD) well. Plate was carefully sealed with parafilm and centrifuge at 32° C.×1300G for 1.5 hr. After centrifugation, parafilm was removed and 500 ul of T-cell media (R10+30 U/ml IL-2) was added to each well, and the plate was incubated. CAR expression FACS analysis was performed on Day 5 post-transduction. 50 ul (~50K) cells per sample were stained for CAR expression using Nectin4-HIS protein (Acro Biosystems #NE4-H52H3, Lot 2823a-214VF1-WR; stock at 0.4 mg/ml) at 2 ug/ml concentration.

Cytotoxicity analysis was performed on Day 12 post-transduction. Briefly, cells were counted using Vi-Cell.

| | Density | |
|---|---|---|
| | (Cells/ml) | Viability |
| T-47D | 1.70E+06 | 91% |
| A431 | 1.35E+06 | 94% |
| OE19 | 1.16E+06 | 92% |
| OVCAR3 | 1.73E+06 | 95% |
| HeLa | 2.32E+06 | 97% |
| K-562 | 8.20E+05 | 99% |

4×10⁶ target cells were centrifuged at 300×g for 5 minutes, and resuspended at 1×10⁶/mL in CTV stain (1:1000 in PBS). Cells were incubated for 10 minutes at 37° C. with inversion of tube at the 5 minute mark to ensure equal staining. CTV stain was quenched with 5 ml R10 and the cells were spun down. Cells were washed with 5 ml R10 media, resuspended in 5 ml R10, and re-counted on Vi-cell.

| | Density | |
|---|---|---|
| | (Cells/ml) | Viability |
| T-47D | 6.70E+05 | 92% |
| A431 | 7.60E+05 | 94% |
| OE19 | 8.80E+05 | 90% |
| OVCAR3 | 6.20E+05 | 94% |
| HeLa | 7.30E+05 | 96% |
| K-562 | 7.10E+05 | 97% |

Target cell density was adjusted to 0.1×10⁶ cells/ml using media (RPMI+10% HI-FBS). Cells were plated at a density of 10,000 target cells per well. Primary transduced T cells (effector cells) were also plated, and 200 ul aliquots of cells from the 6 well plates were collected and counted using Vi-Cell.

| | | Cell | |
|---|---|---|---|
| | | density | Viability |
| 6-well; | UTD | 1.03E+06 | 97% |
| Primary | P3106 | 8.60E+05 | 97% |
| T cells | P3107 | 1.17E+06 | 96% |
| | P3108 | 1.10E+06 | 96% |
| | P3109 | 1.00E+06 | 96% |
| | P3110 | 9.60E+05 | 97% |
| | P3112 | 9.30E+05 | 96% |
| | P3113 | 9.30E+05 | 96% |
| | P3114 | 9.60E+05 | 96% |
| | P2025 | 9.10E+05 | 96% |
| | P2026 | 8.80E+05 | 96% |

Effector cell density was adjusted to ~0.2×10⁶ cells/ml using media (RPMI+10% HI-FBS) based on CAR+ expression. 100 ul or target cell suspension were plated in 96 well flat bottom tissue culture plates.

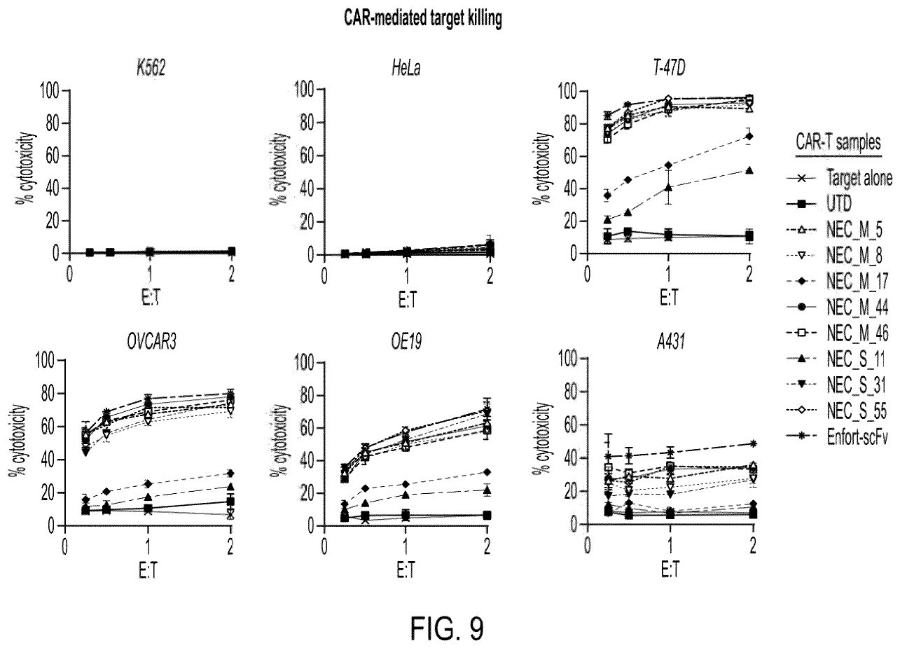

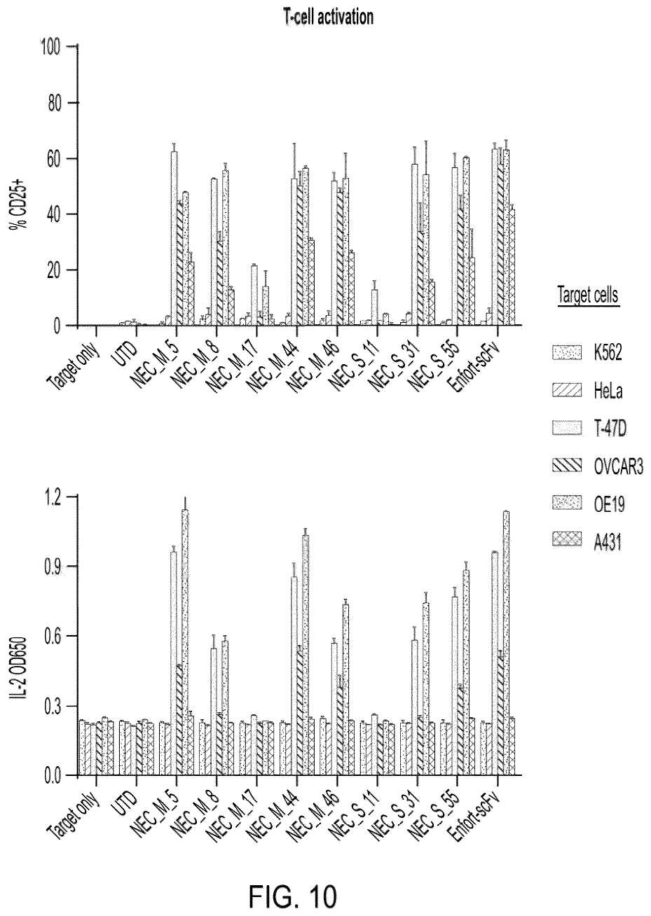

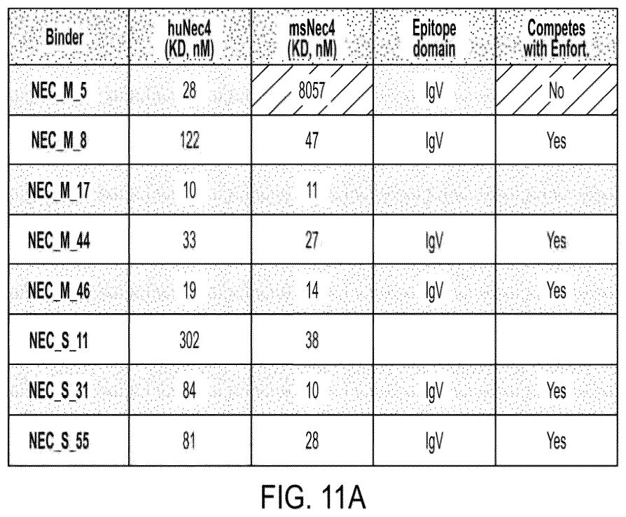

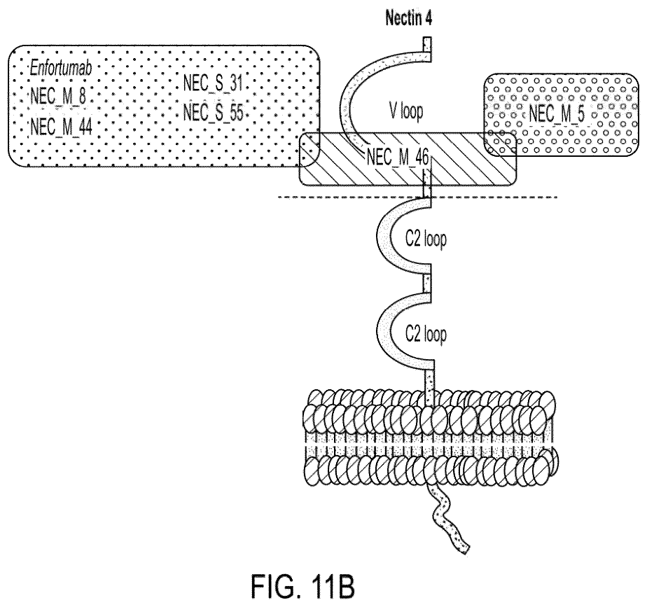

FIG. 9 shows the results of the cytotoxicity assay. VHH-CAR T-cells demonstrated target-specific killing of Nectin4+ cell lines in vitro. CAR expression confirmed for all constructs, ranging from ~50-90% expression. CAR-T cell samples demonstrated binding to recombinant Nectin4-HIS protein, and binding was proportional to CAR expression (VHH Geomean). P3108 and P3112 had lower CAR expression and minimal binding to recombinant Nectin4-HIS protein, Nectin4 VHH-CAR and positive control scFv-CAR demonstrated CAR-mediated cytotoxic activity against Nectin4+ tumor cell lines (T-47D, OVCAR3, OE19, and A431). No off-target killing observed against Nectin4-negative lines, K562 or HeLa, indicating binder specificity. Overall, 6 anti-Nectin4 VHH CAR-T demonstrate robust killing of Nectin4+ tumor cell lines and are comparable to Enfortumab-scFv/CAR (NEC_M_5, P3106; NEC_M_8, P3107; NEC_M_44, P3109; NEC_M_46, P3110; NEC_S_31, P3113; NEC_S_55, P3114).

### Example 7. Nectin4 Binder Specificity Screening

A cell-based specificity FACS screen was established to support lead VHH characterization and selection. VHH-Fc cell binding dose-response curves (DRC) were generated against a diverse panel of human cell lines derived from various tissue/organ types. VHH-Fc that demonstrate non-specific binding to target-negative lines were flagged for potential off-target binding, whereas VHH-Fc that demonstrate minimal non-specific binding to target-negative lines were advanced.

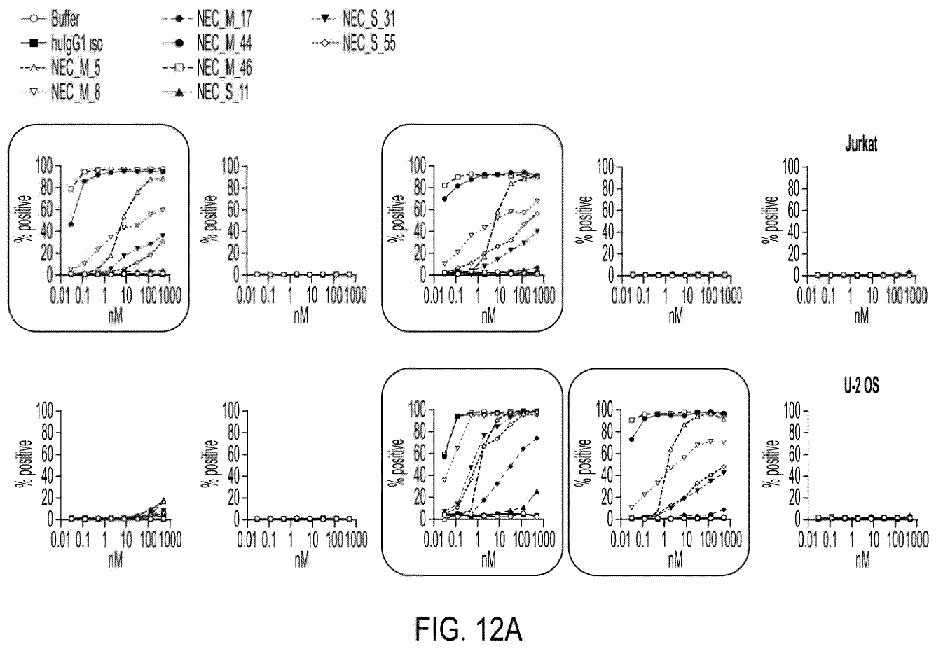

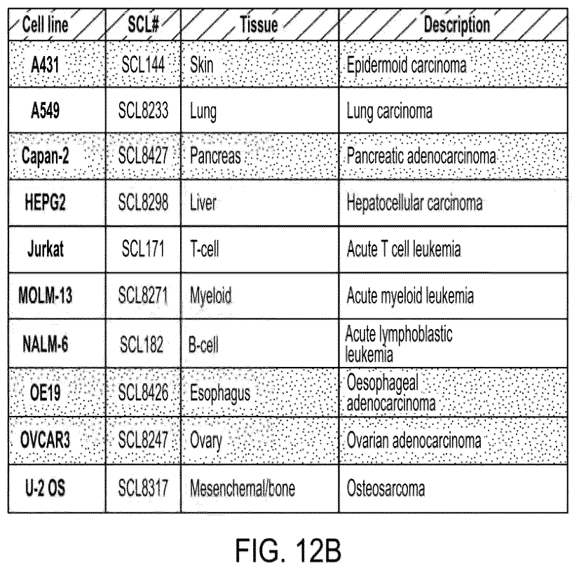

Anti-Nectin4 VHH-Fc cell binders were screened. Specific cell binding was previously confirmed at 5 and 50 nM against CHO-Nectin4 and T47D lines, with no binding to Jurkat or CHO Parental (see, e.g., FIG. 6). Briefly, the FACS screen was performed by harvesting all cells, and plating 50×10³ cells per well. Blocking was performed using InvivomAb Fc block (25 ug/ml for 20-30 min RT). Cells were washed 1×, and incubated in VHH-Fc for 30 min at 4° C. Cells were then washed 3×, and incubated in PE anti-VHH 1 ug/ml+NearIR (1:1000) for 30 min 4° C. Cells were washed 2×, and imaged. The results of a FACs screen are shown in FIG. 12A, and the corresponding description of cell lines used in the specificity screen are provided in FIG. 12B.

A431, Capan-2, OE19, and OVCAR3 cell lines express Nectin4. None of the samples demonstrated substantial non-specific binding to Nectin4-negative lines. When observing Geomean graphs, PROT1735 and PROT1749 exhibited some increased background binding to HEPG2, Jurkat, and U-2 OS. There were notable differences in binding curves to Nectin4+ lines, resulting in a range of EC50 values between clones. Some samples demonstrate weak binding to MOLM-13 at 500 nM, which may be due to presence of FcR and incomplete Fc blocking.

### Example 8. Determining Nectin4 Antigen Density on Target Cell Lines

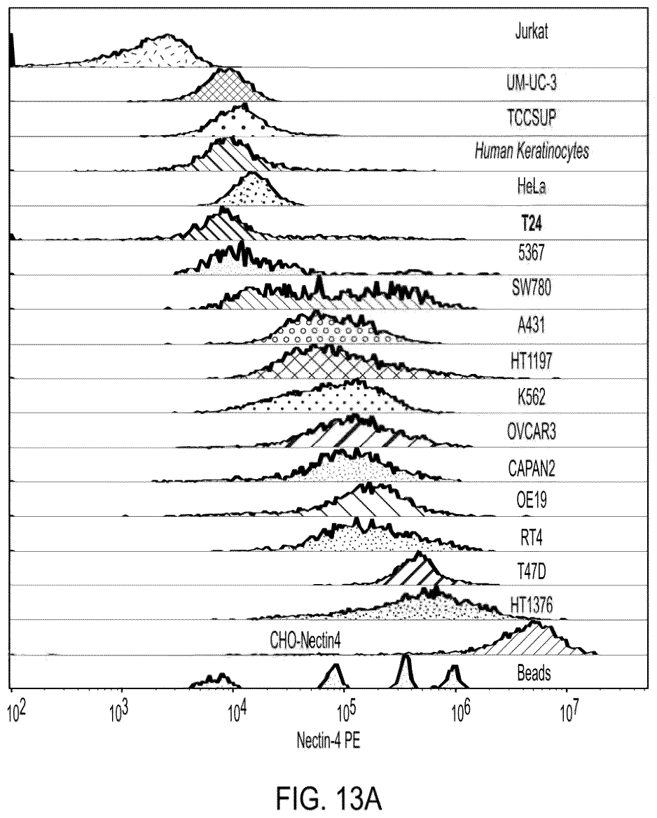

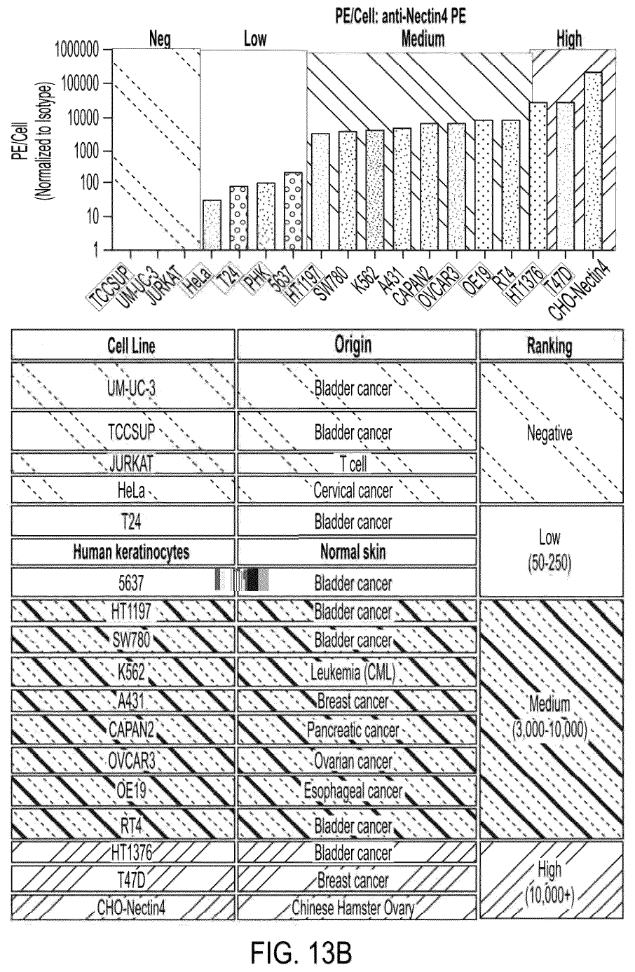

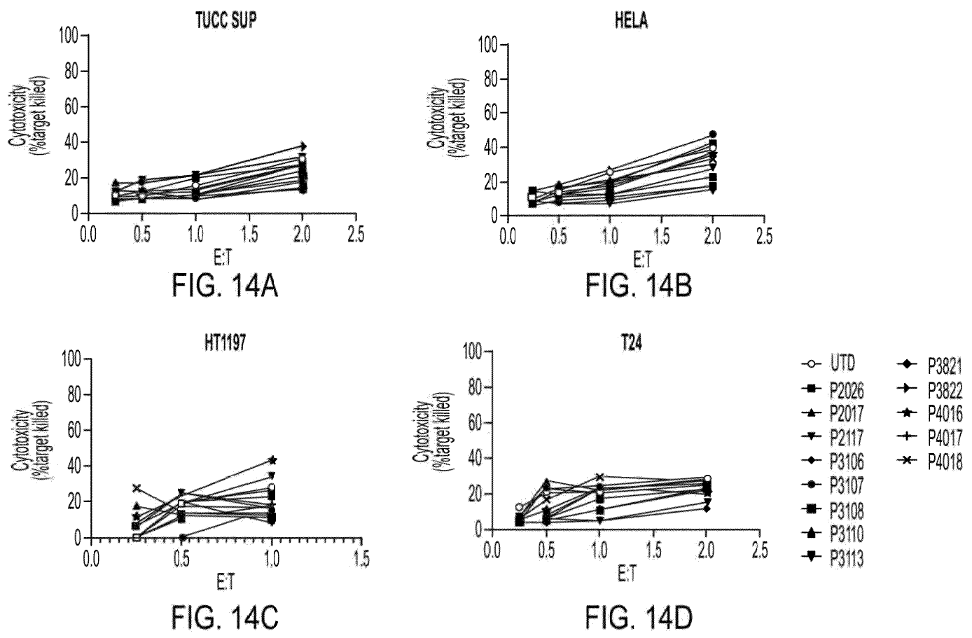

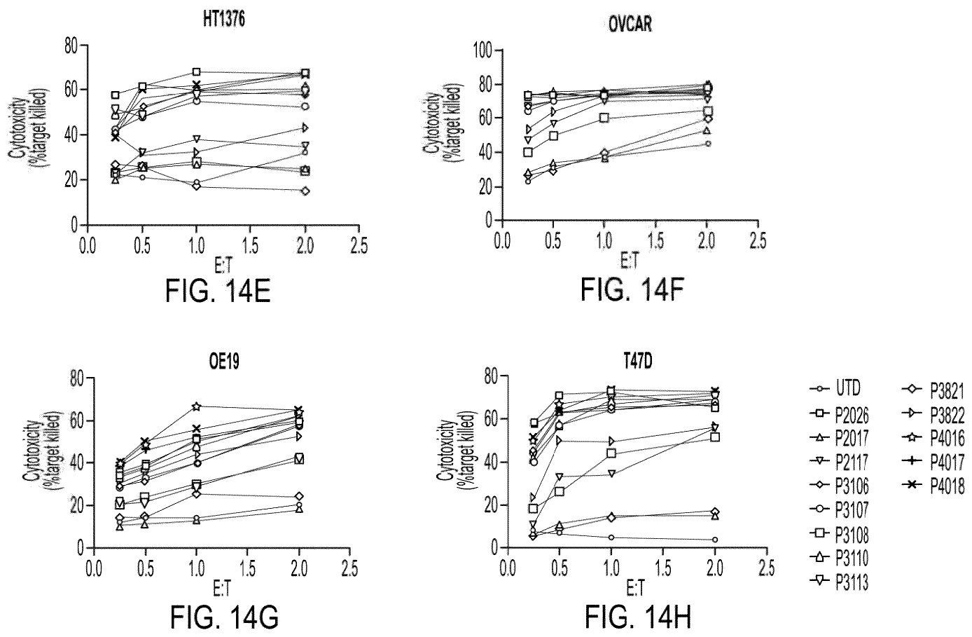

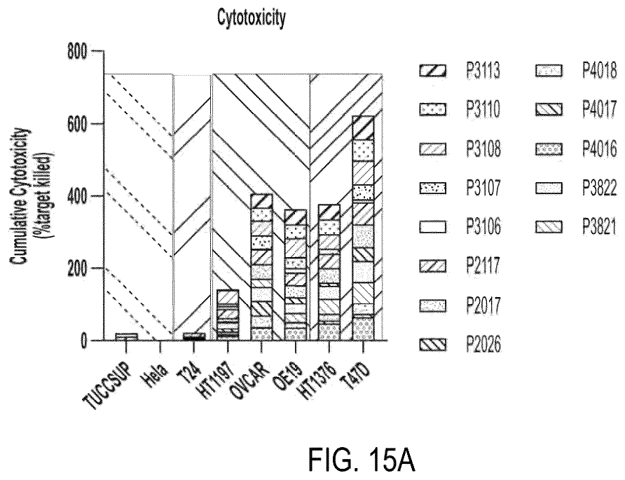

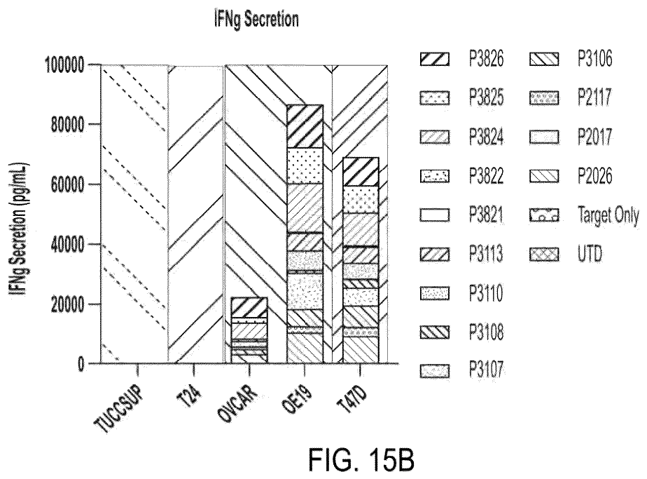

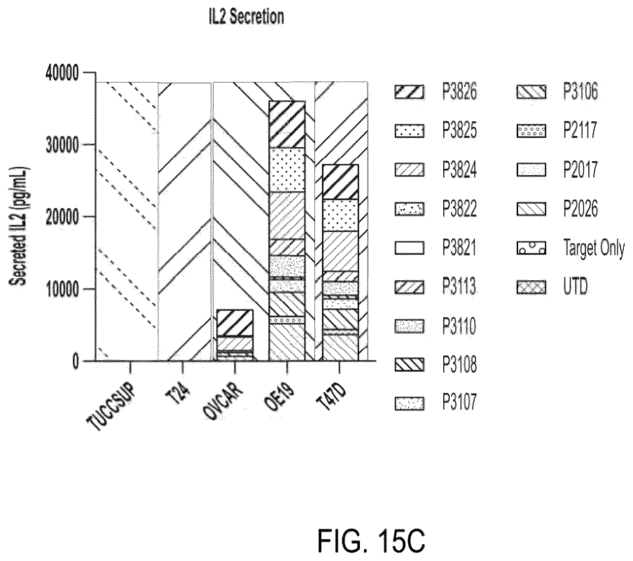

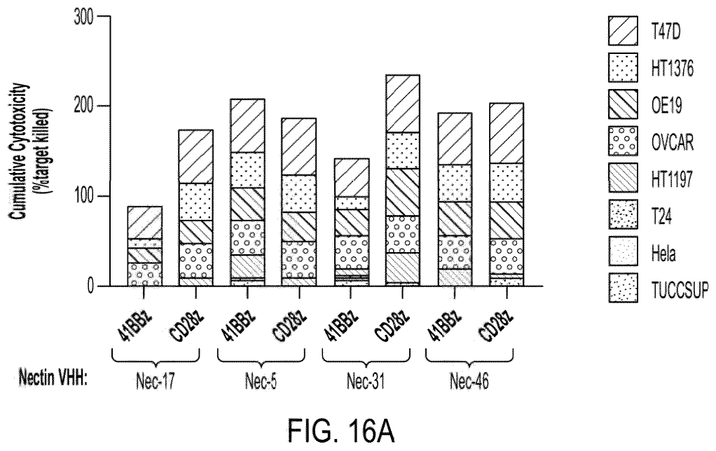

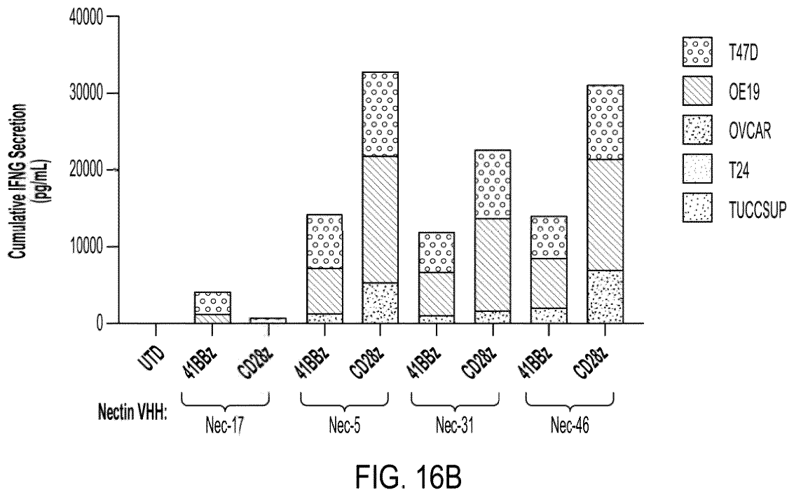

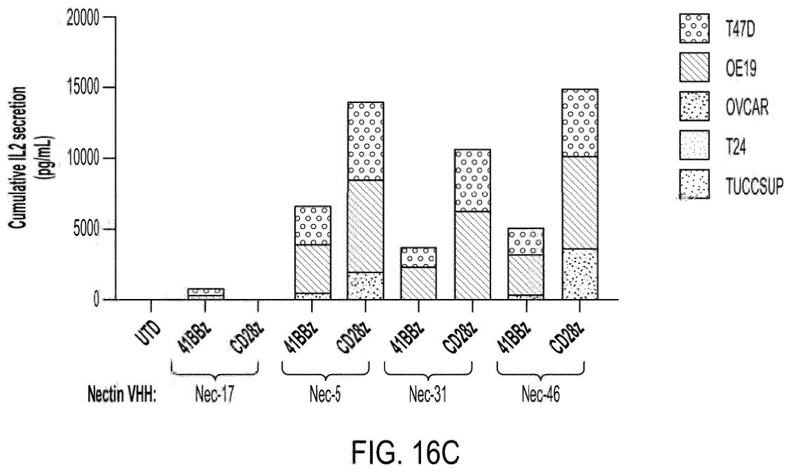

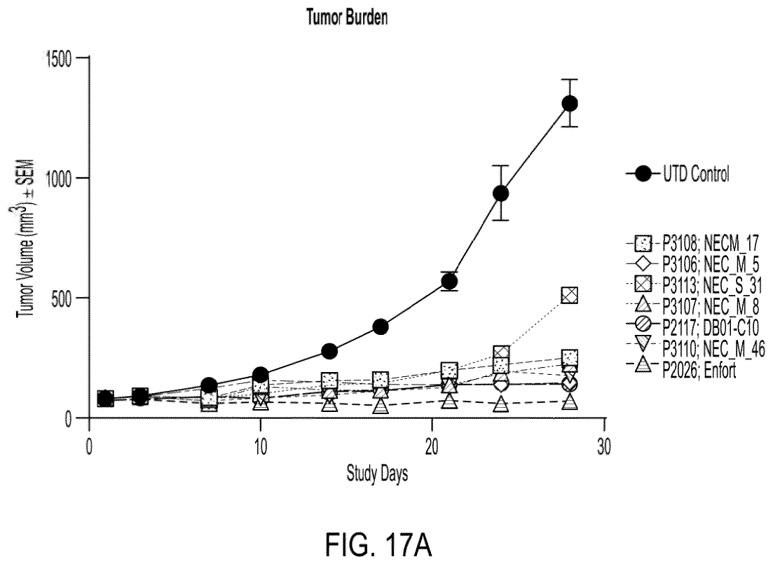

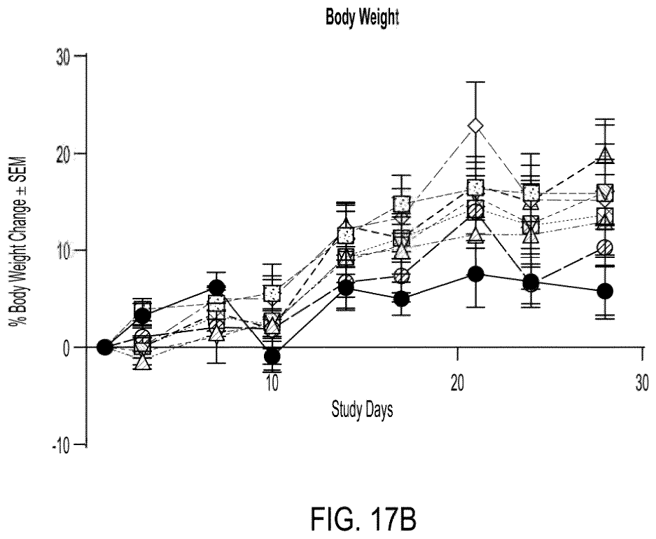

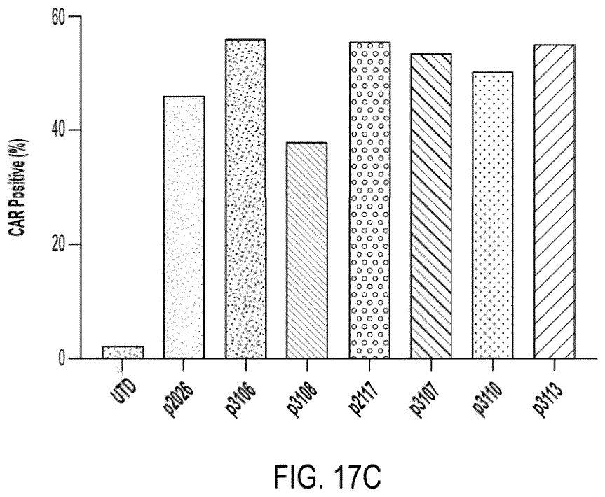

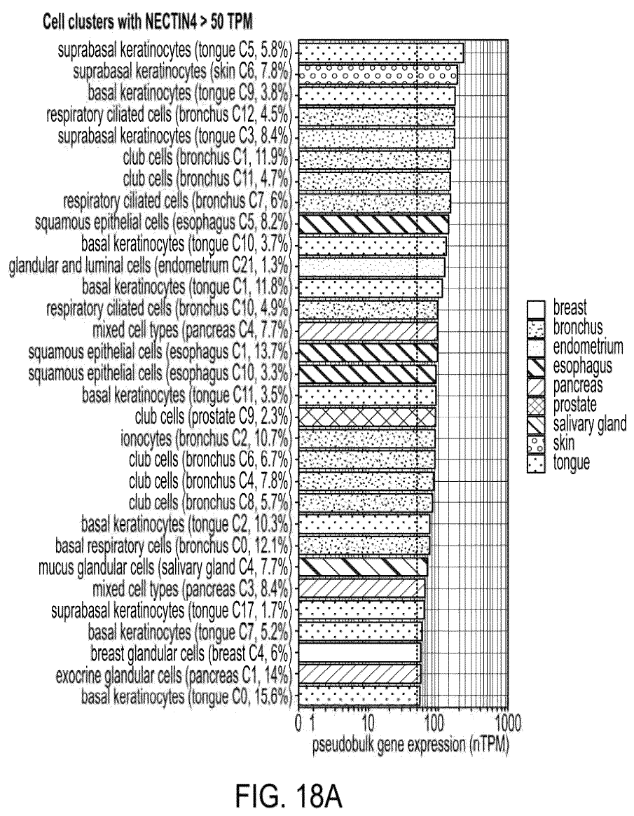

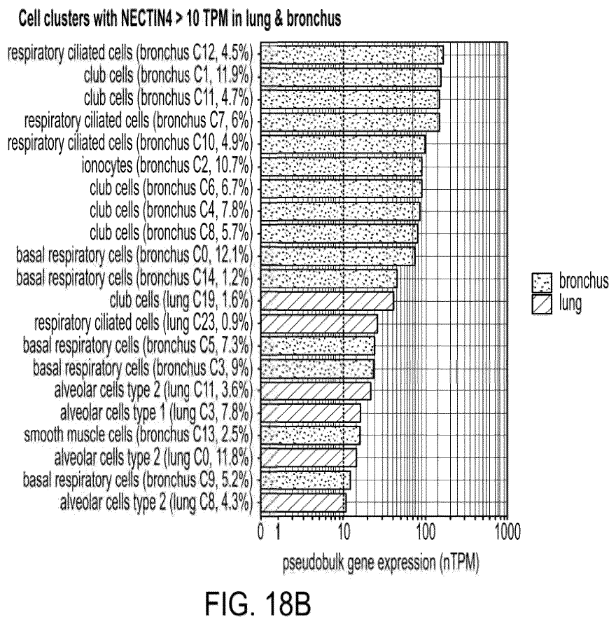

As shown in FIGS. 13A-B, Nectin4 antigen density was assessed on a variety of solid tumor and control cell lines, as well as primary human keratinocytes (PHKs), using the Quantibrite PE kit from BD. Quantibrite beads are coated with 4 calculated levels of PE, low, medium low, medium high, and high. Using these calculated PE/bead values and their fluorescence intensity in flow cytometry, a standard curve was created to estimate the antigen density on cell lines.

### Materials:

McCoy's 5A Medium (Gibco CAT #16600082)
RPMI1640 Medium (Gibco CAT #61870036)
EMEM Medium (ATCC CAT #30-2003)
DMEM Medium (Gibco CAT #10569010)
Lebovitz's L15 Medium (Gibco CAT #11415064)
Accutase (Gibco CAT #00-4555-56)
TrypLE Express (Gibco CAT #12605028)
DPBS (Gibco CAT #14190144)
HI FBS (Gibco CAT #10438026)
BD Staining Buffer, BSA (BD Pharmingen, CAT #554657)
QuantiBrite PE Beads (BD Biosciences CAT #340495, Lot #82820)
anti-human Nectin4, PE conjugated (R & D Systems CAT #FAB2659P)
anti-human Mesothelin, PE conjugated (R & D Systems CAT #FAB32652P)
Mouse IgG2a kappa isotype control, PE (R & D Systems CAT #MAB0031)
Rat IgG2b isotype control, PE (R & D Systems CAT #IC006P)
Human Fc-G1 (Fc block)(BioXCell CAT #BE0096, Lot #74782001

### Procedure:

Flow cytometry was performed to assess Nectin4 expression. Briefly, cells were washed twice with 150 uL BD stain buffer. Cells were Fc-receptor blocked in 50 uL stain buffer with 25 ug/mL BioXCell Human Fc-G1. Cells were incubated at room temperature for 25 min, and washed twice with 150 uL BD stain buffer. Mouse IgG2b anti-human Nectin4 PE, mouse IgG2b isotype PE, Rat IgG2a anti-human Mesothelin PE, and Rat IgG2a isotype PE were diluted 1:25 in BD stain buffer. Cells were resuspended in 50 uL stain, and incubated for 30 min in the fridge. Cells and beads were washed twice with 150 uL BD stain buffer, and resuspended in 30 uL stain buffer and run on the intellicyt iQue3 flow cytometer.

A PE/cell calculation was also performed. From the Quantibrite kit, the PE/bead values for the four PE levels, and the geomean of their PE signal from the flow cytometer, were obtained. The log 10 value of each was taken, and used to create a standard curve and best fit line. Geomeans of the PE signal for each cell line were averaged for each duplicate and normalized to the average signal yielded from the isotype stain, and applied to this standard curve to yield their estimated PE/cell. Final calculated PE/cell are provided in FIG. 13B. The solid tumor cell lines chosen for this study demonstrate a range of expression values for Nectin4, from negative to very low to medium to high. This information can be used to design experiments to assess the sensitivity of various Nectin4 VHH binders.

### Example 9. Nectin4 CAR Xenograph Tumor Growth Inhibition Efficacy Study in Mice Implanted with OVCAR-3 Tumor Cells

45 animals were implanted with 5×10⁶ cells/mouse in 1:1 RPMI:Matrigel SC. Initial body weight (BW) measurements were taken, and then re-taken 2× weekly for the duration of the study. Tumor volumes were collected 2× weekly after study randomization. On day 1, dose levels were normalized to 37% (P3108) using the UTD cells. Untransduced T cells (same donor) were used to supplement CAR-T cell suspensions so that total number of T cells was equal for all groups.

All treatment groups show a statistically significant tumor growth inhibition (TGI) on day 28. P3108, P3106, P3107, P2117, and P3110 exhibit TGI similar to the positive control, Enfortumab. P3113 exhibits a reduction in efficacy on d28, compared to other treatments. Statistical analysis was performed in Prism using Linear Mixed Effect analysis of the log transformed data.

### Example 10. Comparison of Suprabasal Keratinocyte and Tumor Gene Expression

The objectives of this experiment were to compare gene expression in normal suprabasal keratinocytes to patient-wise median gene expression in tumor tissues, and to conduct the analysis for all protein coding genes that display on the cell surface and for a basket of Nectin4 expressing cancer indications including bladder cancer, breast cancer, esophageal cancer, head and neck cancers, non-small cell lung cancer, and ovarian cancer.

### Materials:

Single cell RNAseq atlas of normal human tissues: RNA single cell type tissue cluster data for 30 normal human tissues from the Human Protein Atlas. A dataframe included:
Gene name
Tissue
Cell type cluster
Read count
Expression in units of Transcripts Per Million that has been normalized to integrate the 30 separate tissue datasets (nTPM)

Bulk RNAseq atlas of cancer tissues: The Cancer Genome Atlas—TCGA. Dataset included 20,000 primary cancer and matched normal samples spanning 33 cancer indications. A dataframe included the following information:
Gene name
Patient ID
Cancer indication
Tissue type (tumor, tumor adjacent, or matched normal)
Read counts
Expression in units of Transcripts Per Million (TPM)

List of surfaceome genes: A list of empirically and/or computationally predicted genes that are displayed on the cell surface, such as the Bausch-Fluck, D. et al. “The in silico human surfaceome” October 2018, 115 (46) E10988-E10997, which is incorporated by reference herein in its entirety.

### Methods:

Prepare the bulk RNAseq atlas of cancer tissue data. Filter by Tissue type=tumor, cancer indication of interest, and gene name in the list of surfaceome genes. One entry was kept per patient ID. The data was grouped by gene name and cancer indication and the median of gene expression was calculated.

Prepare the single cell RNAseq atlas of normal human tissue data. Filter by Tissue Type=skin, cell type cluster=suprabasal keratinocyte, and gene names in the list of surfaceome genes.

Join the patient-wise median tumor gene expression and normal skin suprabasal keratinocyte datasets. The two datasets were prepared as described above, and joined by gene name.

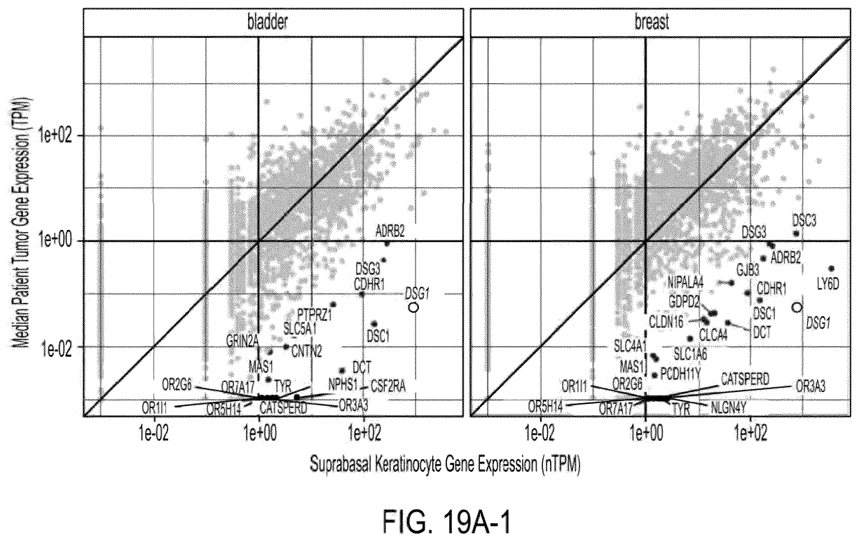

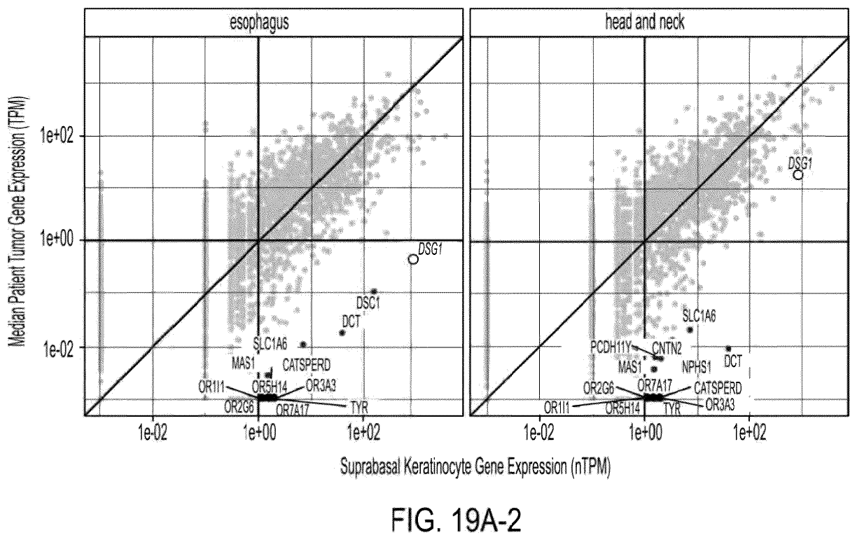

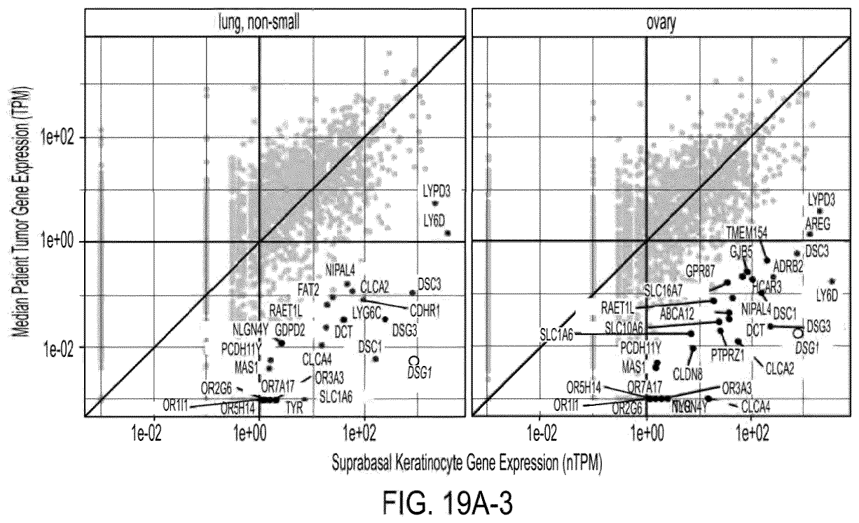

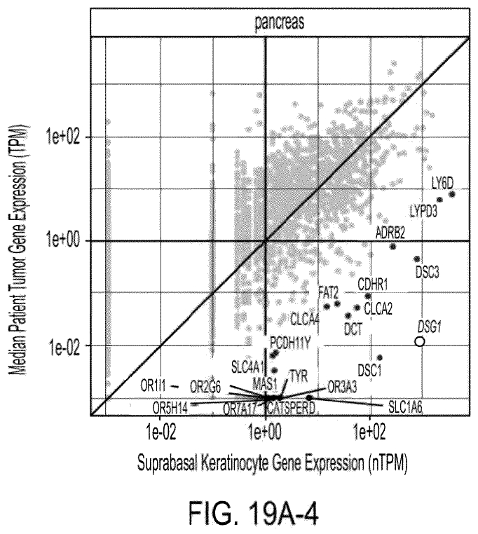

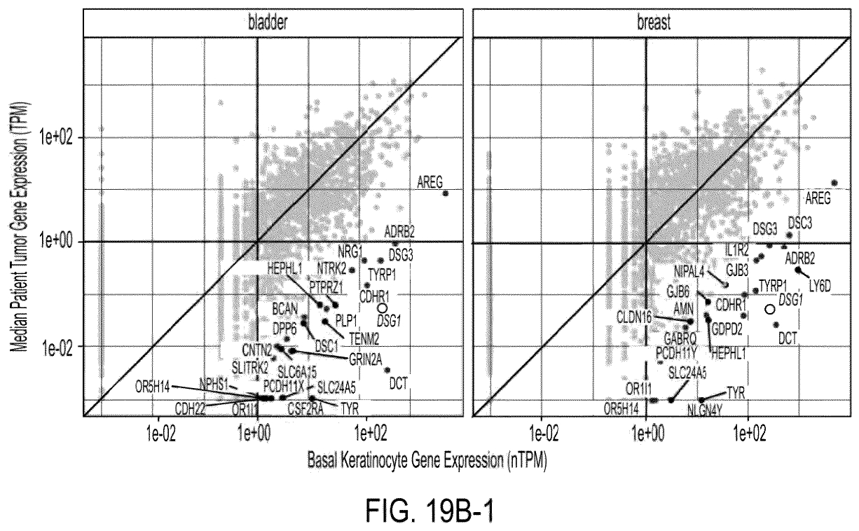

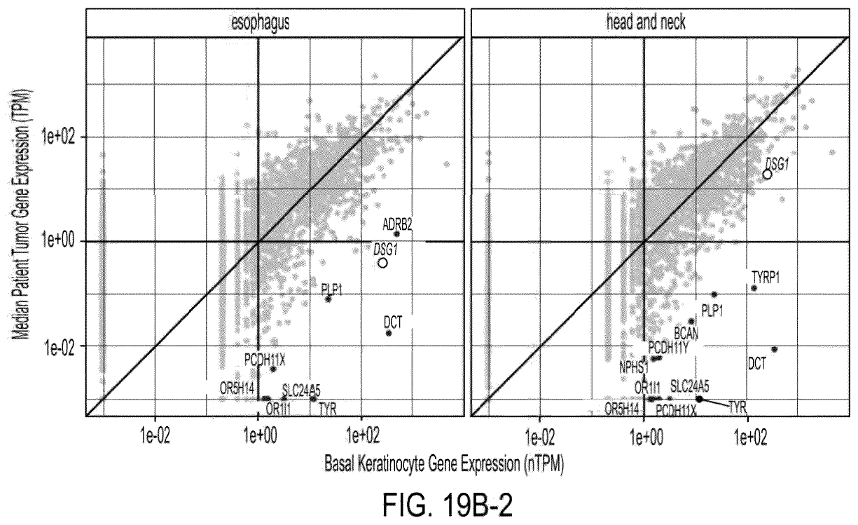

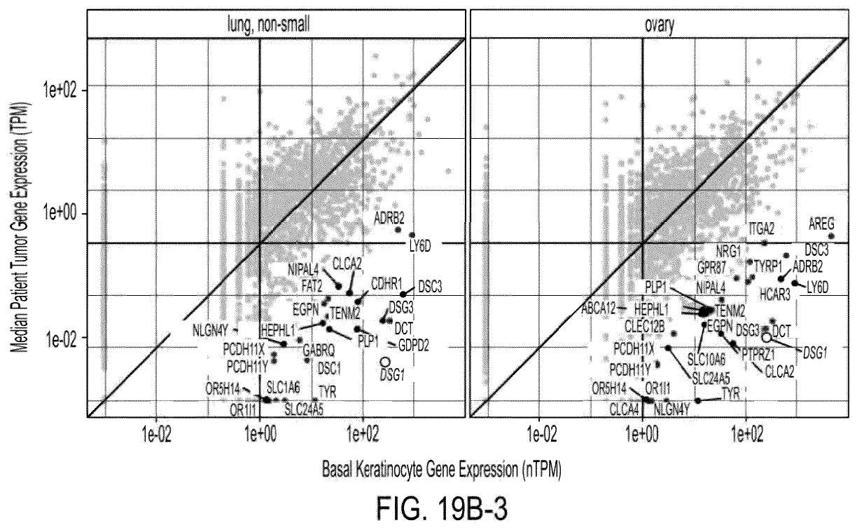

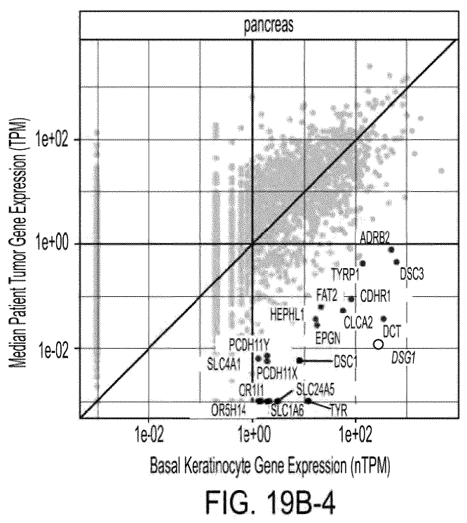

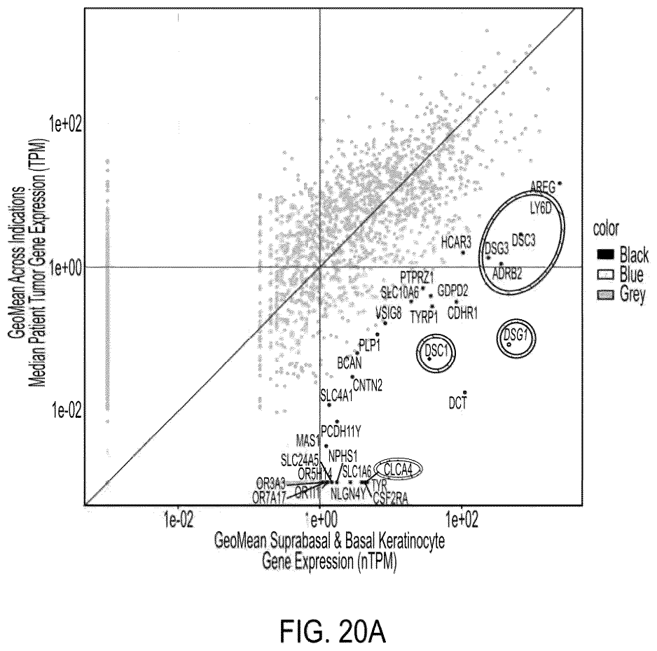

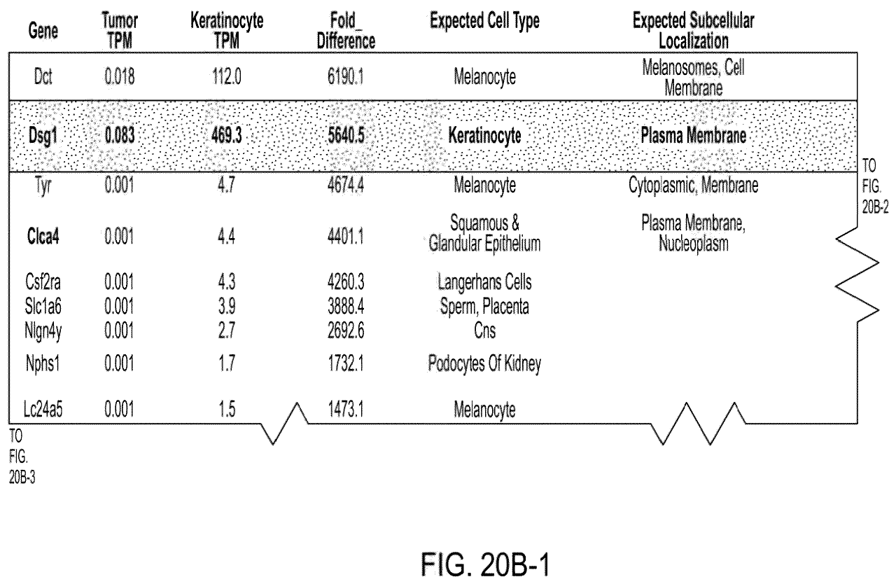

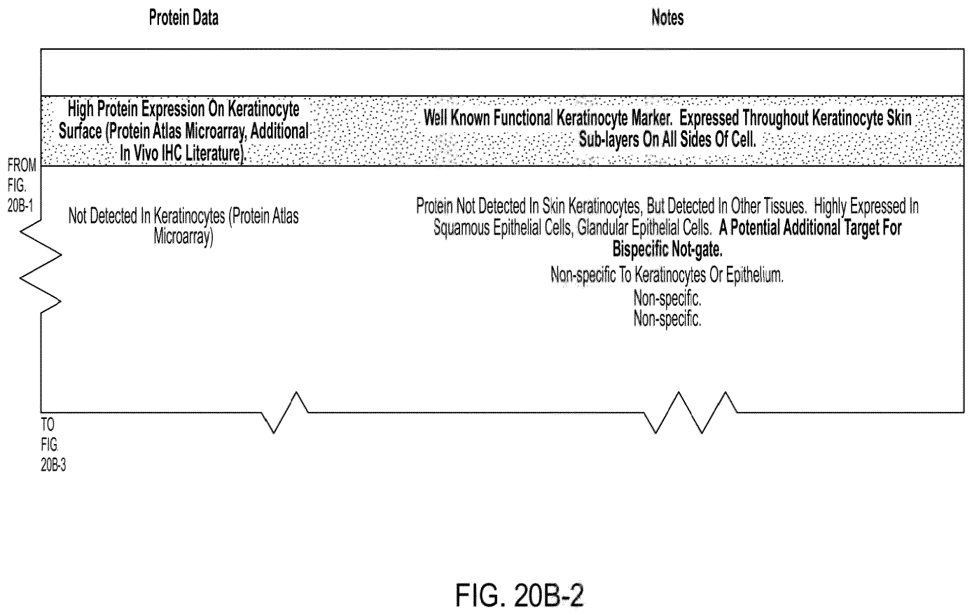

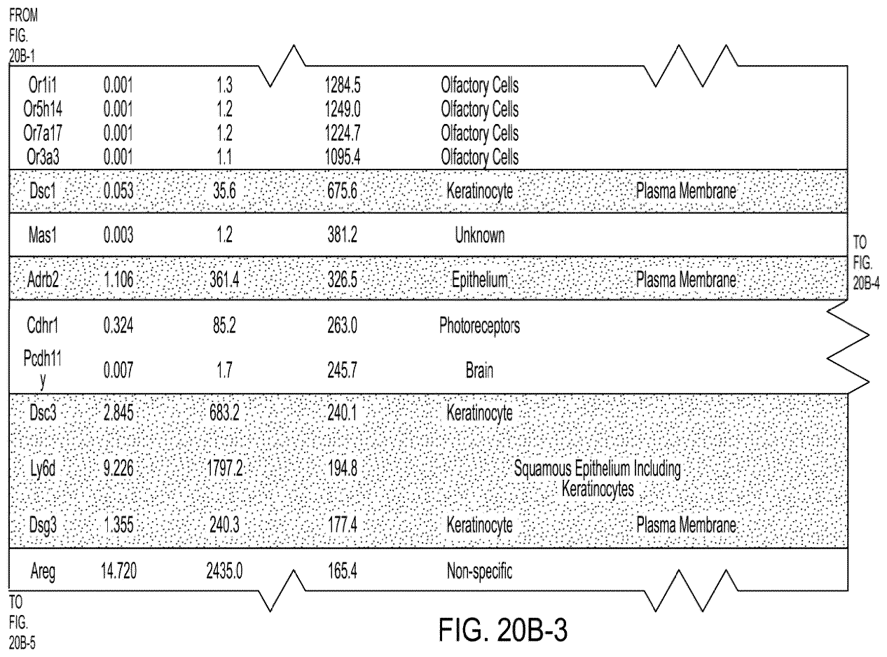

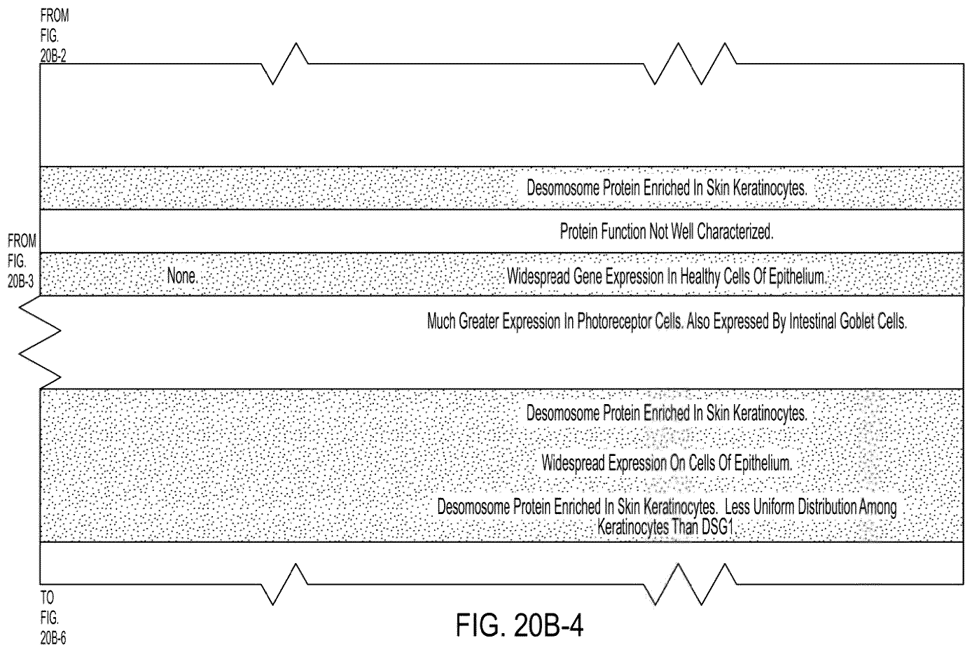

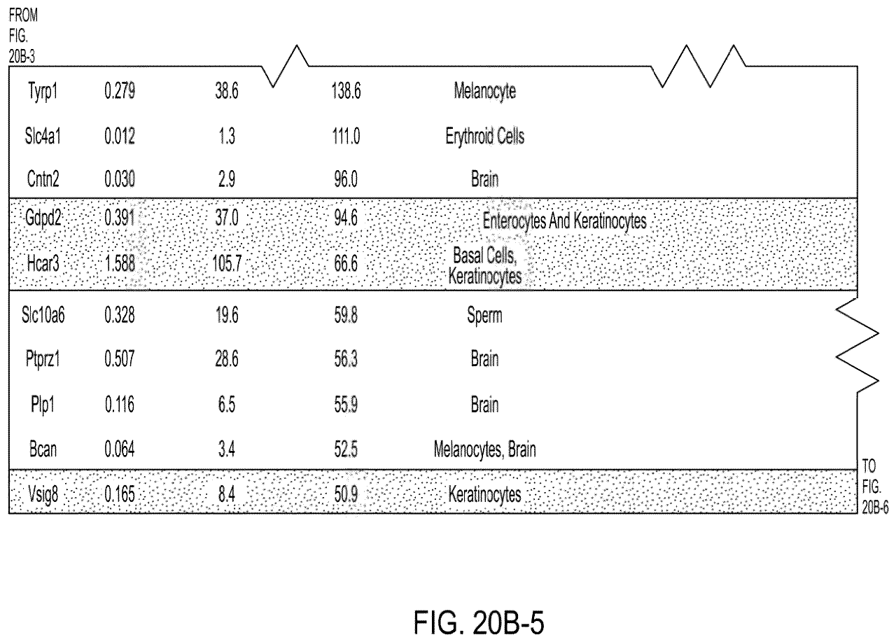

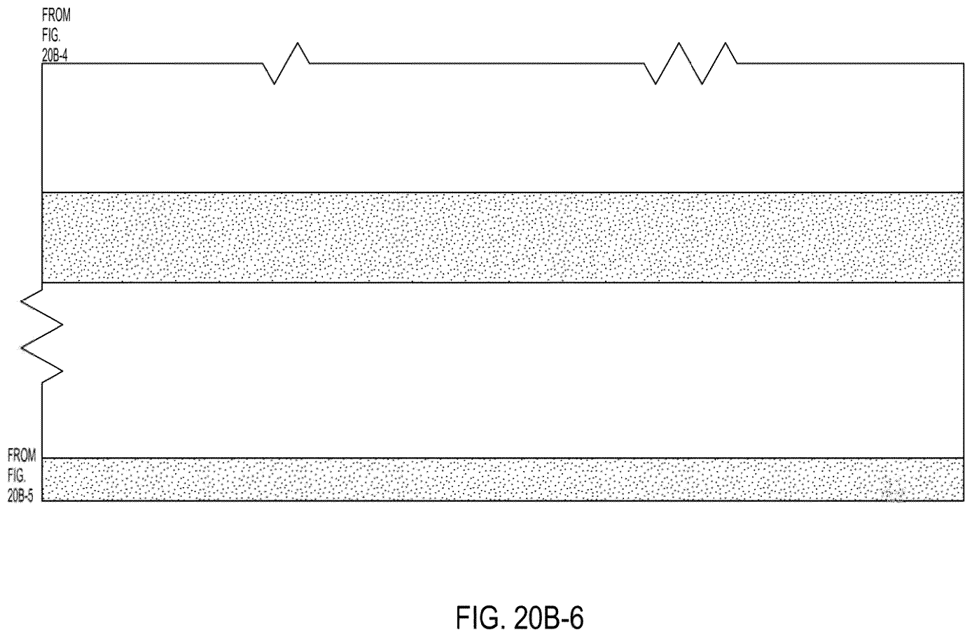

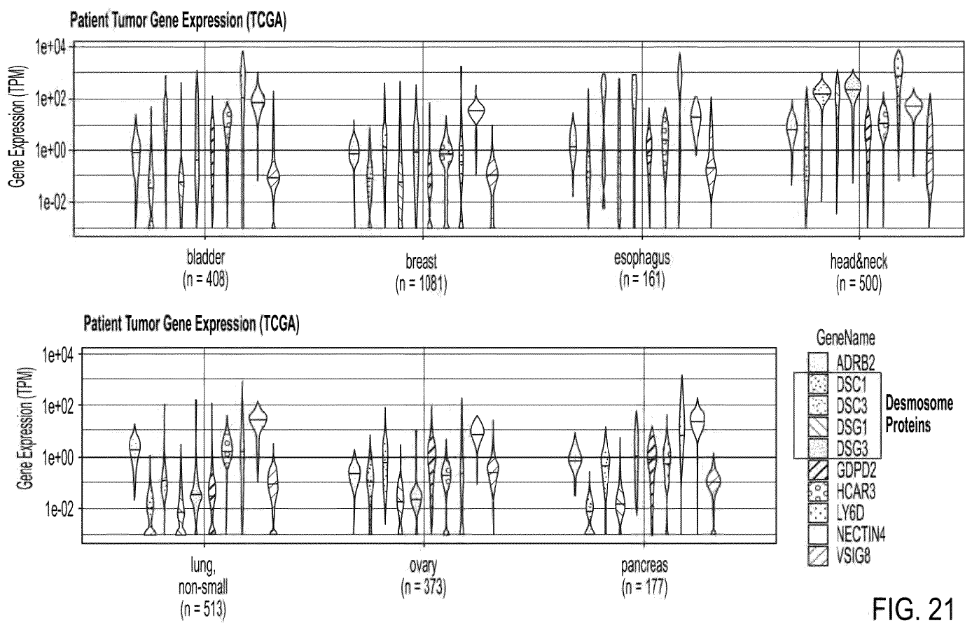

Visualize. The data was visualized as shown in FIG. 19A, which shows a scatter plot of the normal skin suprabasal keratinocyte vs. tumor gene expression while faceting by cancer indication.

### Conclusion:

DSG1 was an outlier (see the lower right quadrant of FIG. 19A), indicating it has much greater gene expression in normal skin suprabasal keratinocytes than in the median patient for several Nectin4 expressing cancer indications, a rare quality among cell surface displayed genes.

### Example 11. Distribution of DSG1 vs. NECTIN4 Gene Expression Across Individuals

The objectives of this experiment were to assess the distribution of DSG1 vs. Nectin4 gene expression across individuals in healthy skin and in tumor tissues from a basket of Nectin4 expressing cancer indications, and to assess the distribution of co-expression of DSG1 and NECTIN4 within the same patient tumor.

### Materials:

Bulk RNAseq atlas of cancer tissues: The Cancer Genome Atlas—TCGA. Dataset included 20,000 primary cancer and matched normal samples spanning 33 cancer indications. A dataframe included the following information:
Gene name
Patient ID
Cancer indication
Tissue type (tumor, tumor adjacent, or matched normal)
Read counts
Expression in units of Transcripts Per Million (TPM)

Bulk RNAseq atlas of normal tissues: Genotype-Tissue Expression project—GTEx. Samples were from 54 non-diseased tissue sites across approximately 1000 individuals. A dataframe included the following information:
Gene name
Subject ID
Tissue
Read counts
Expression in units of Transcripts Per Million (TPM)

### Methods:

Prepare the bulk RNAseq atlas of cancer tissue data. Filter by Tissue type=tumor, cancer indication of interest, and gene name=DSG1 or NECTIN4. One entry was kept per patient ID.

Prepare the bulk RNAseq atlas of normal tissues. Filter by Tissue type=skin sun exposed or skin not sun exposed, and gene name=DSG1 or NECTIN4. One entry was kept per patient ID.

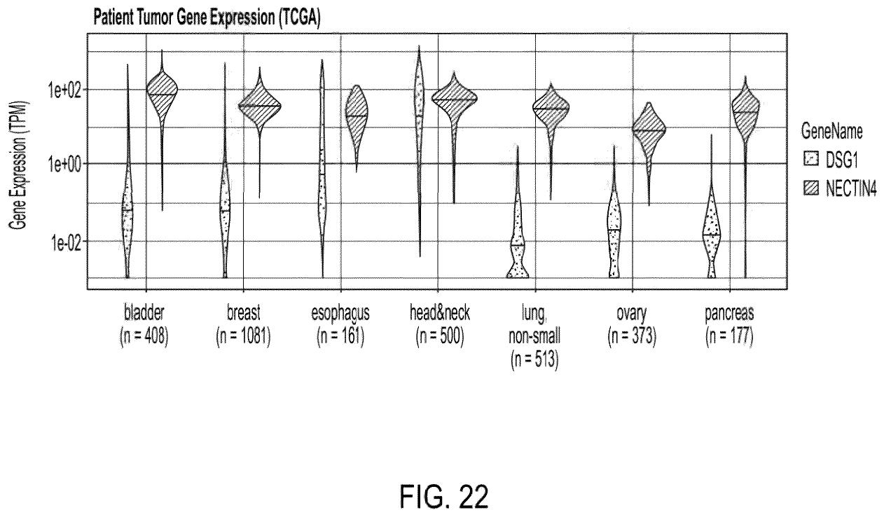

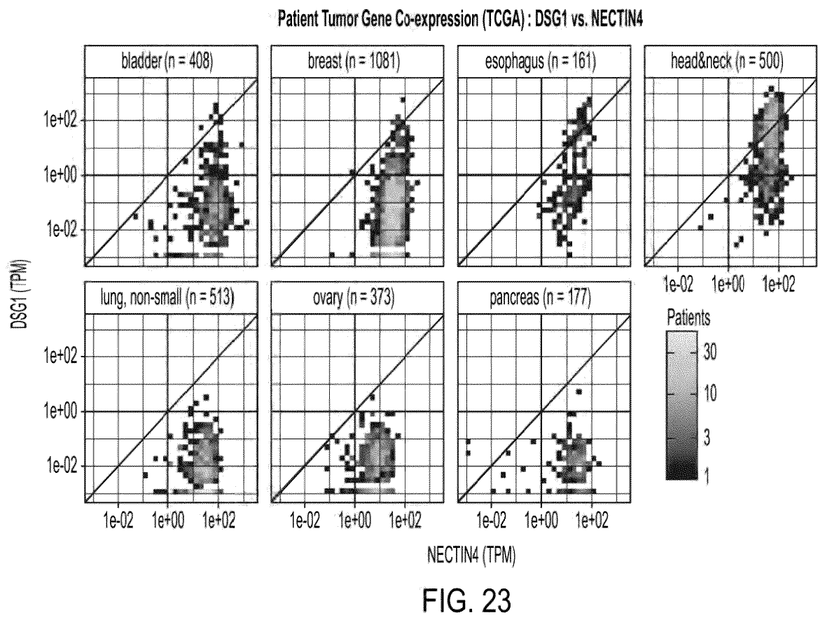

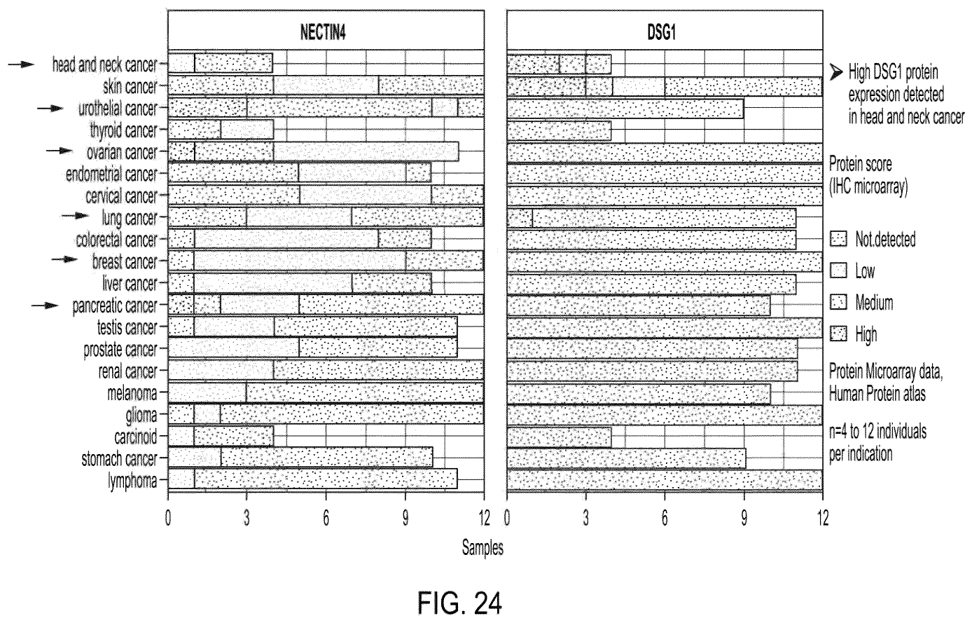

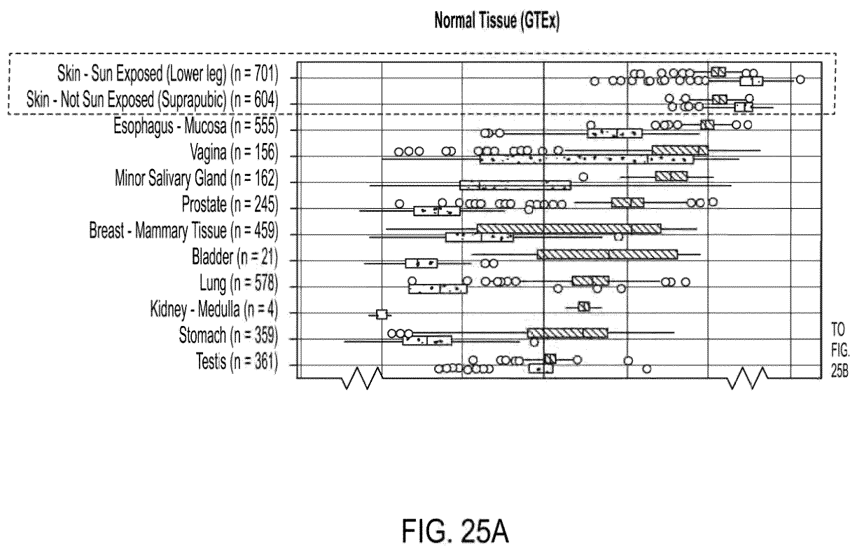

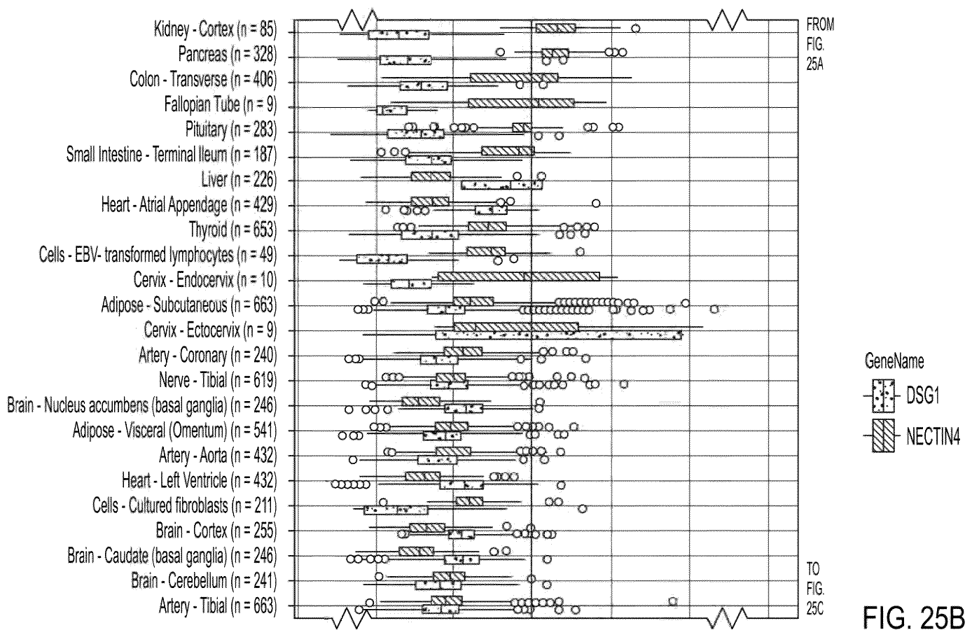

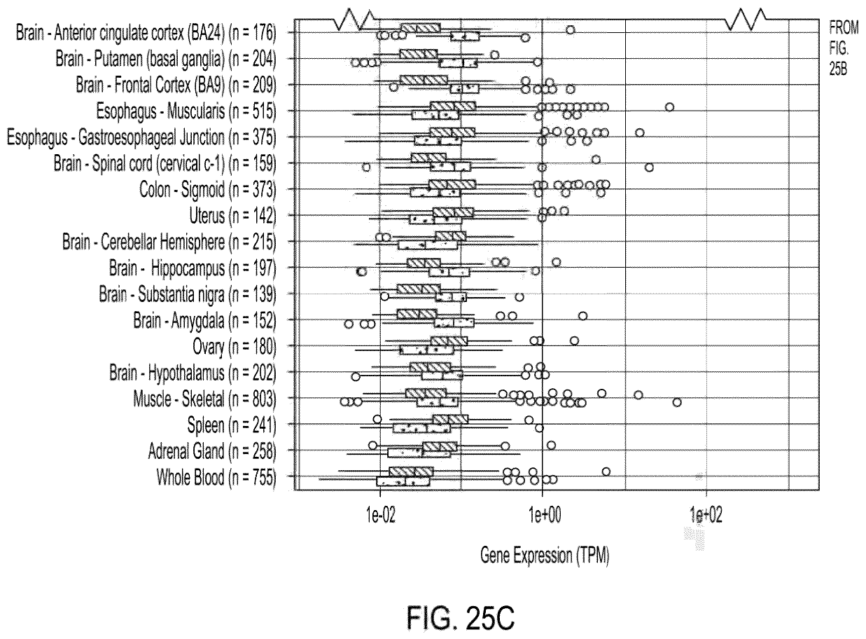

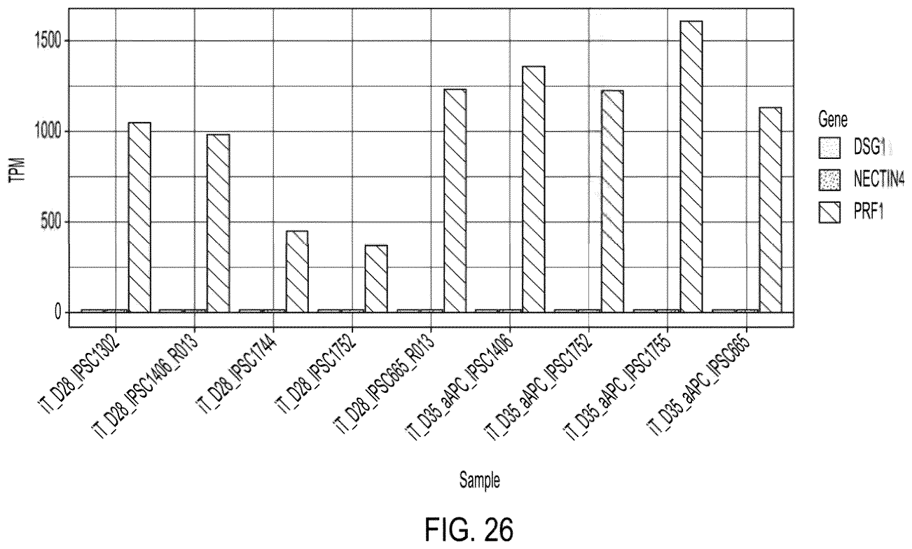

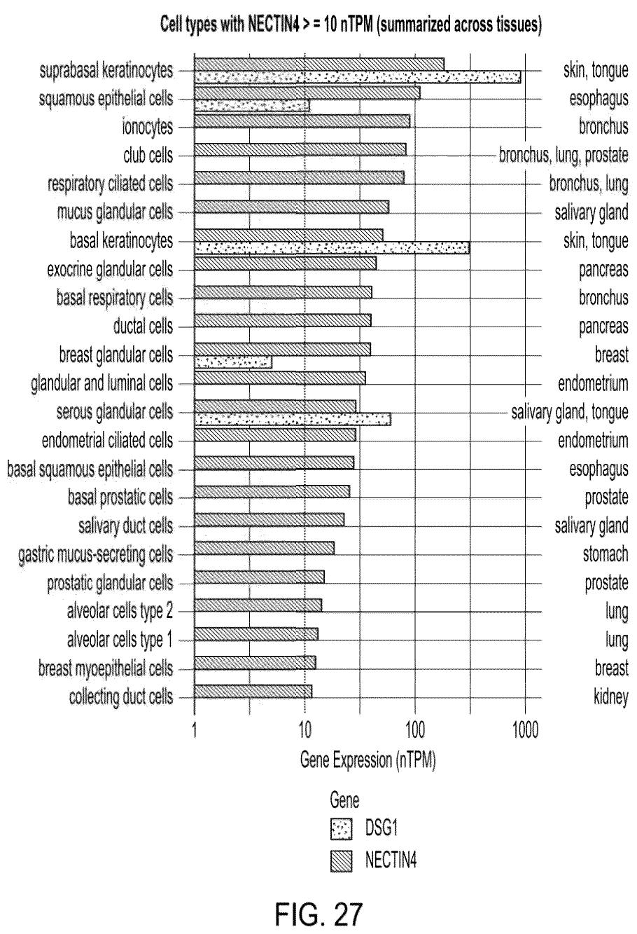

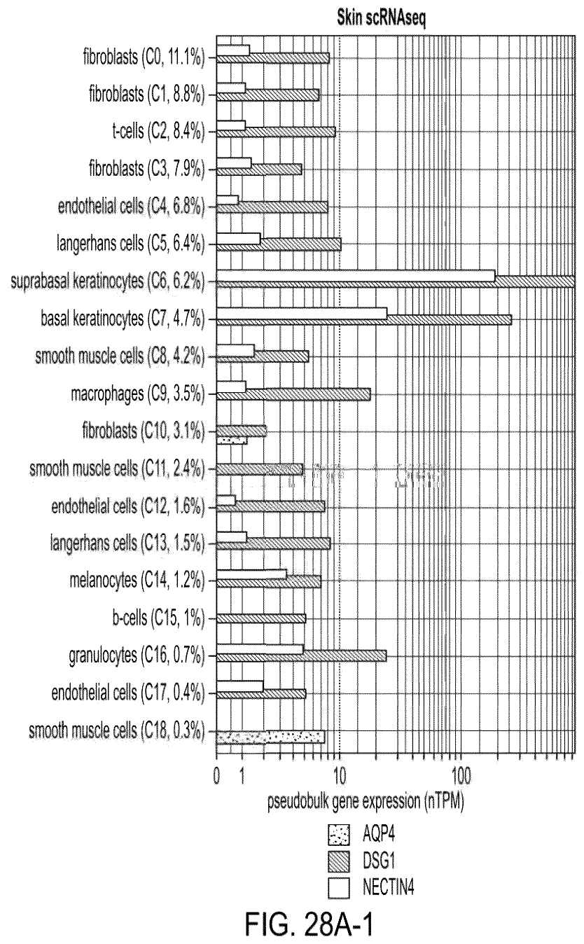

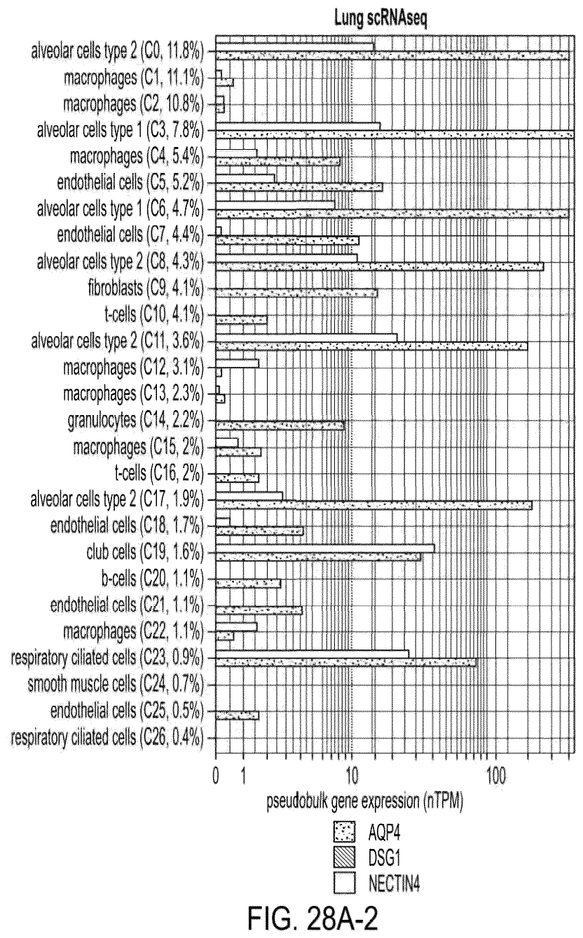

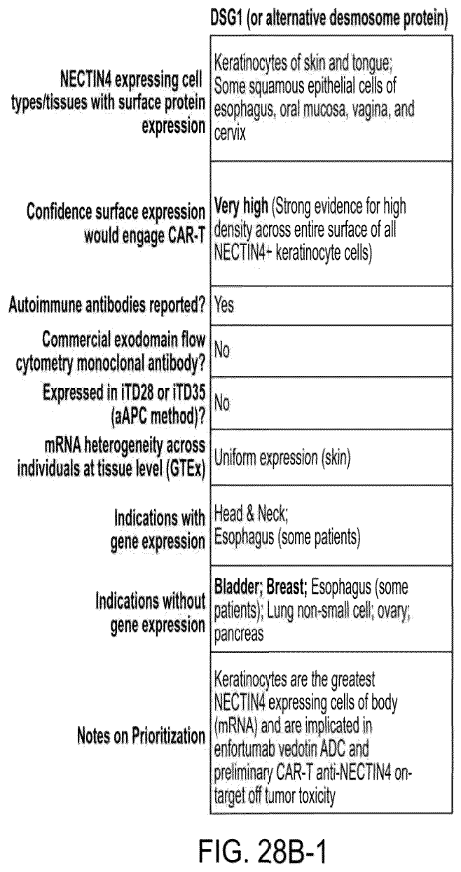

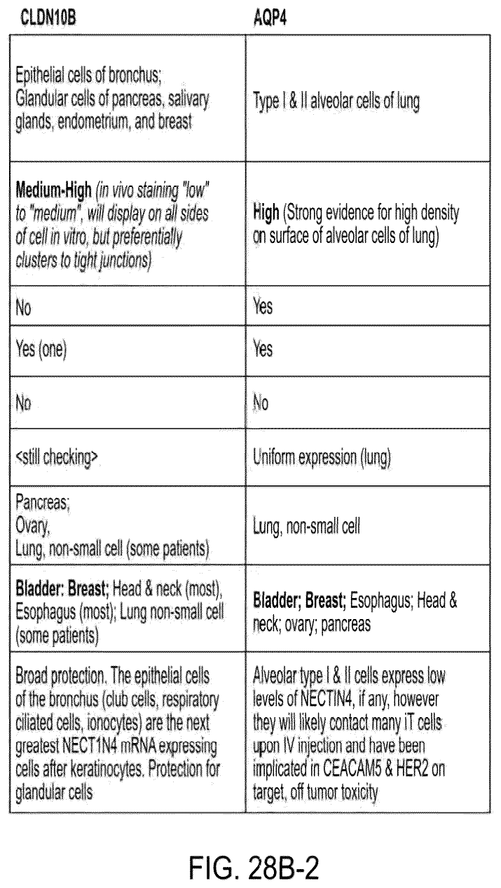

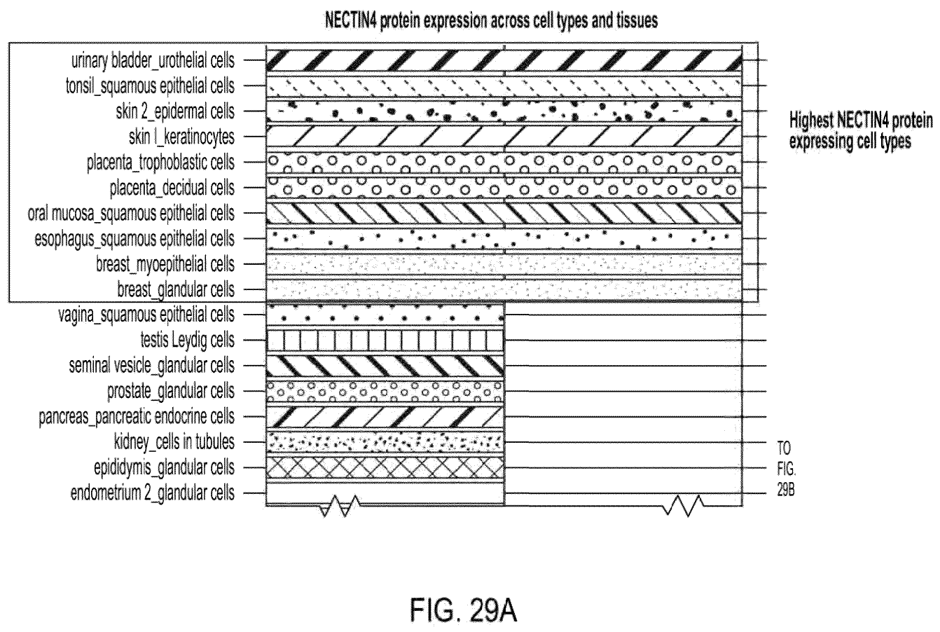

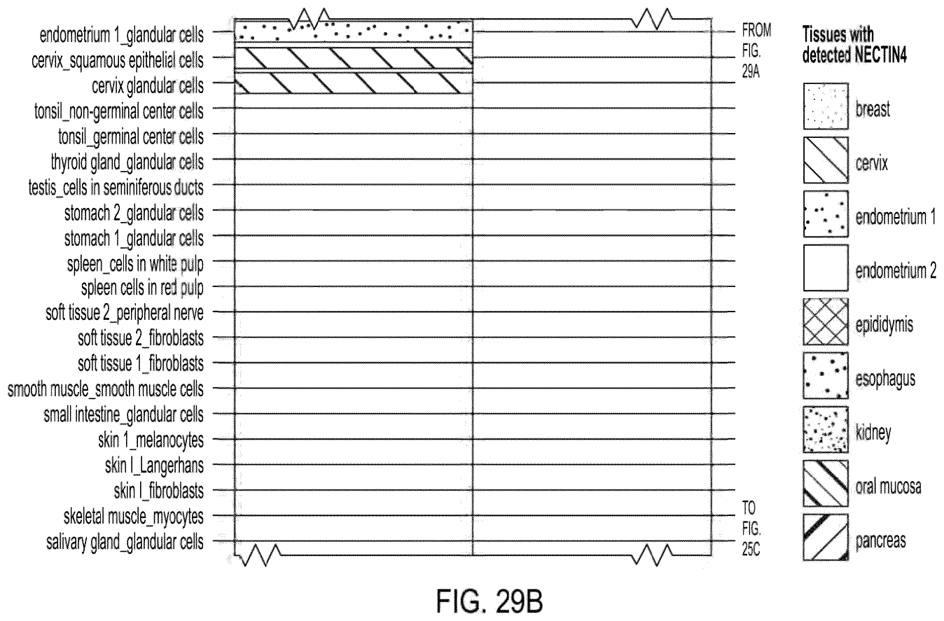

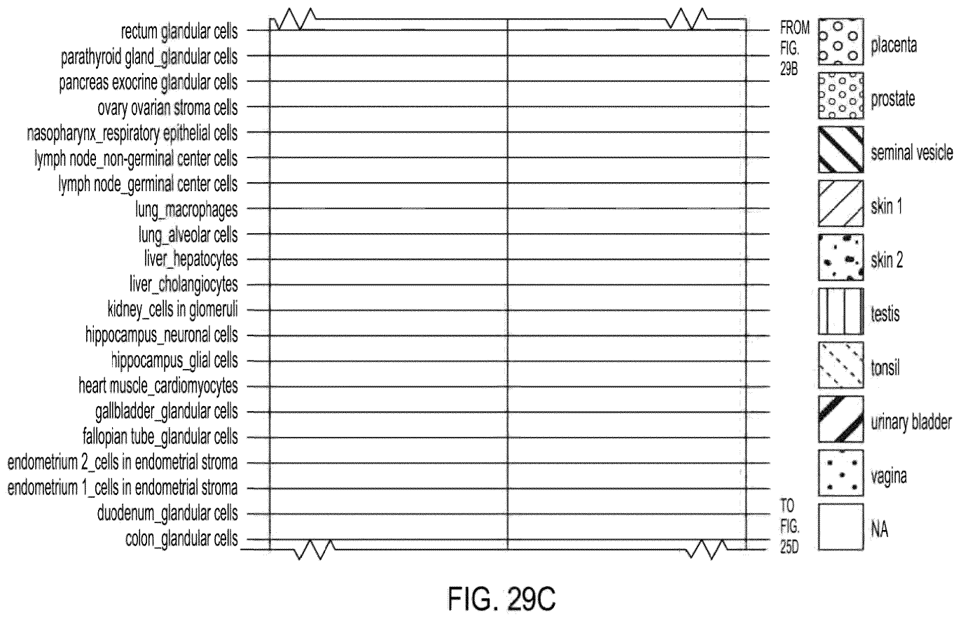

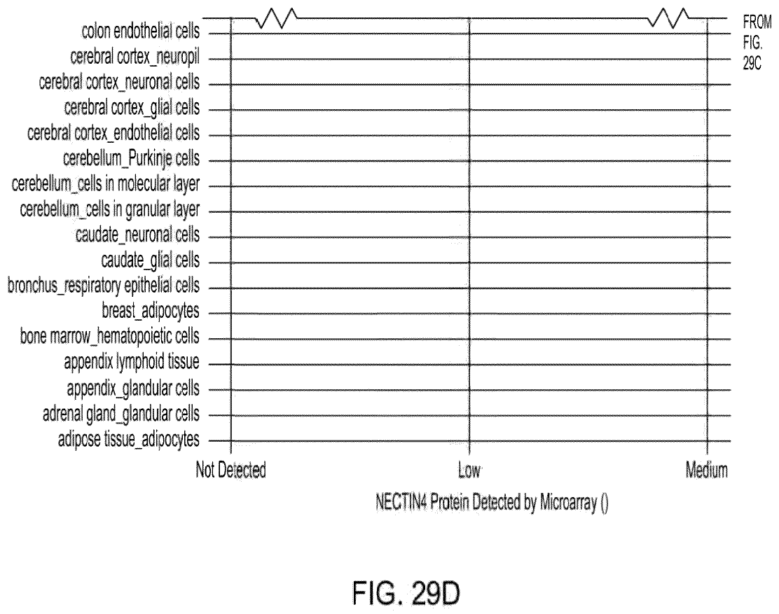

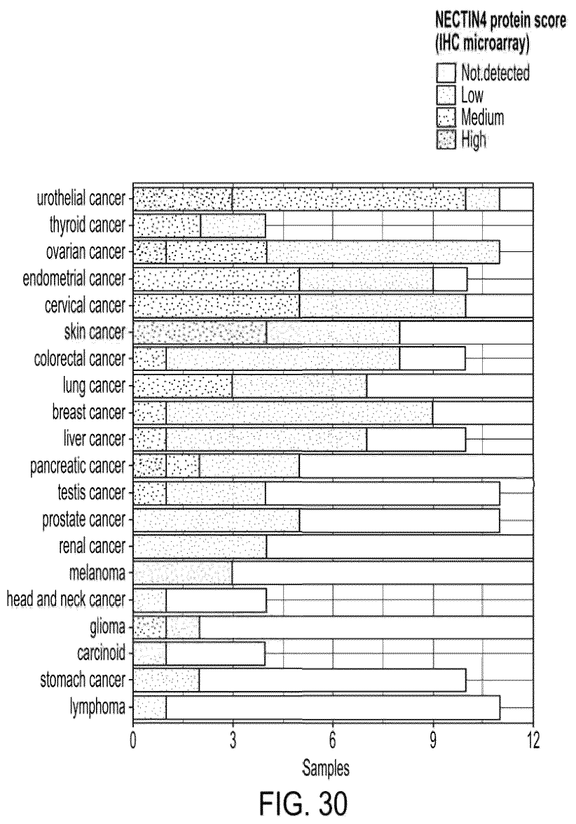

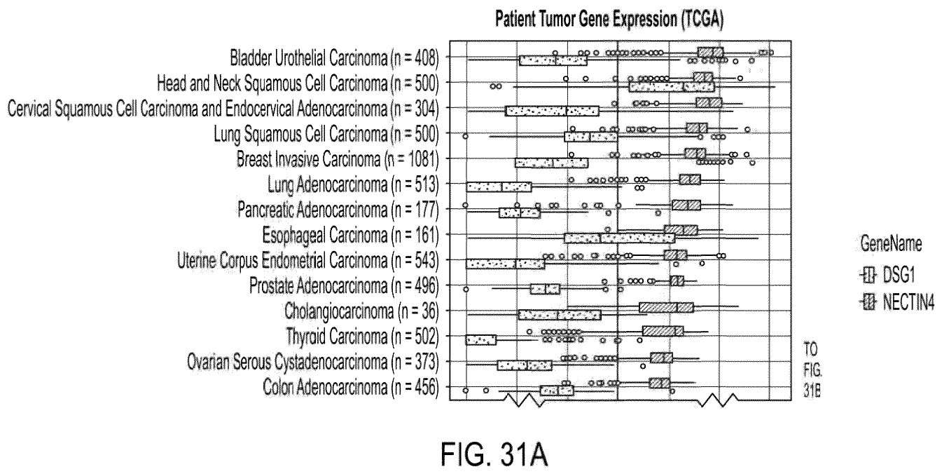

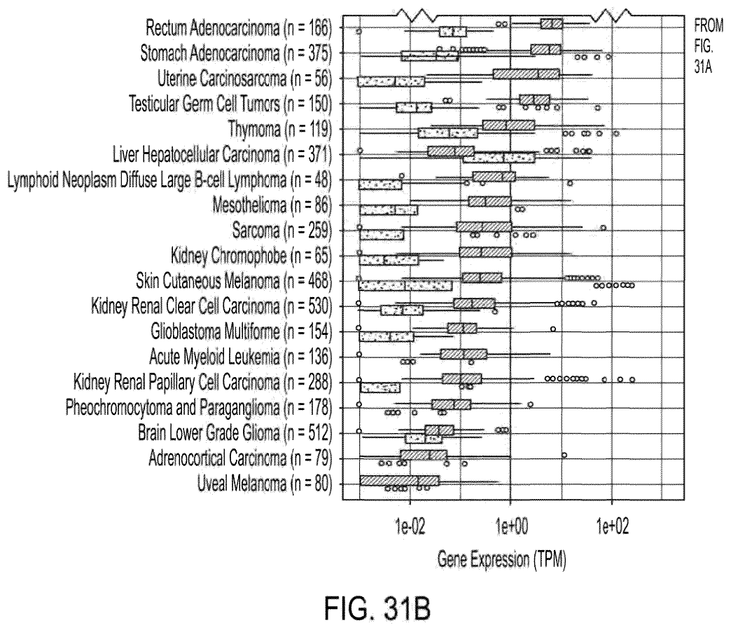

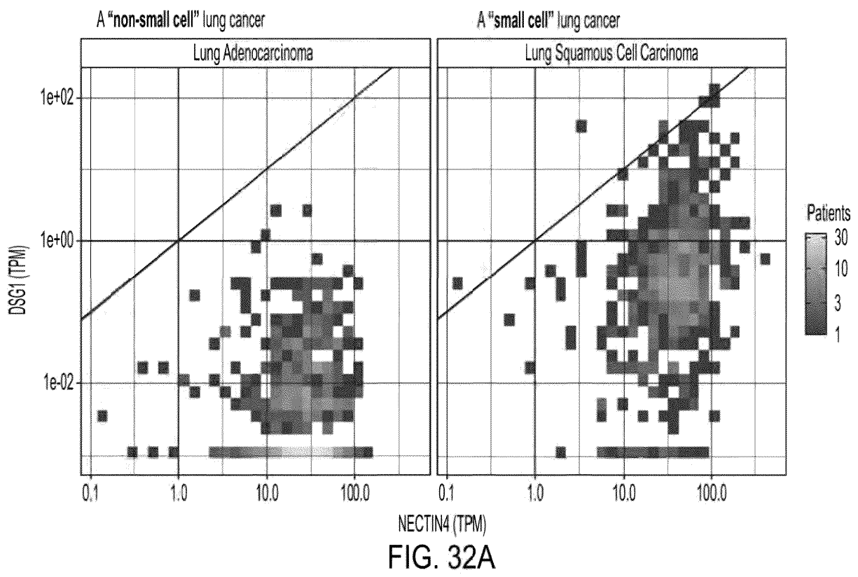

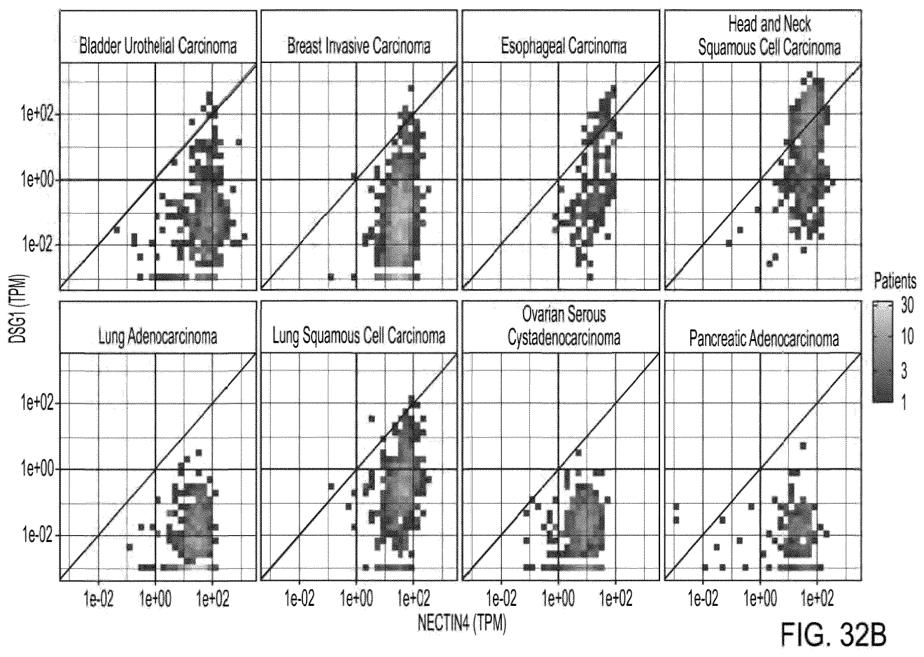

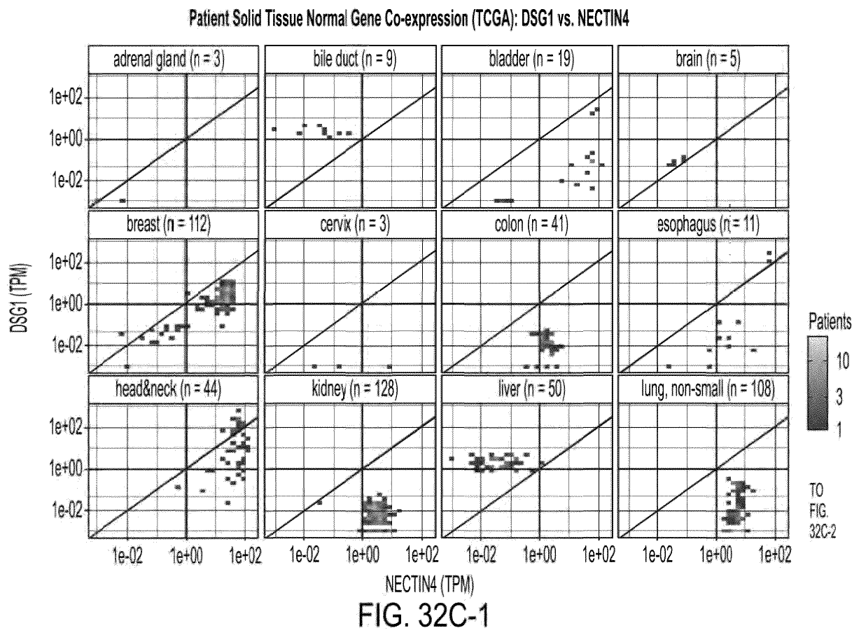

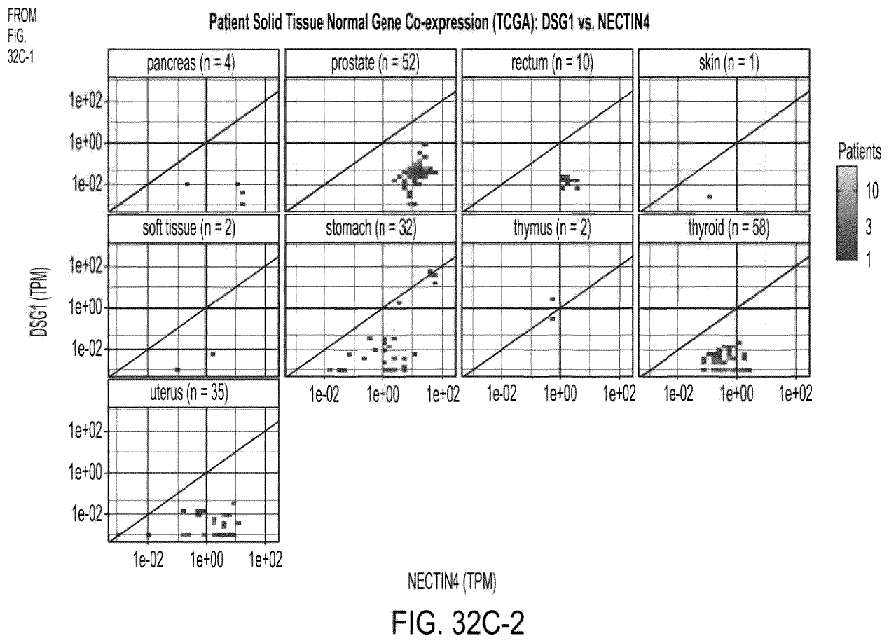

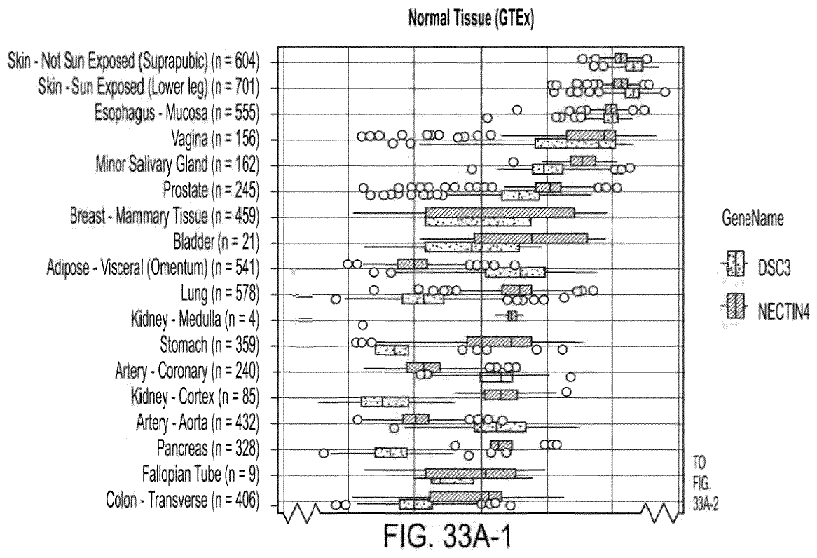

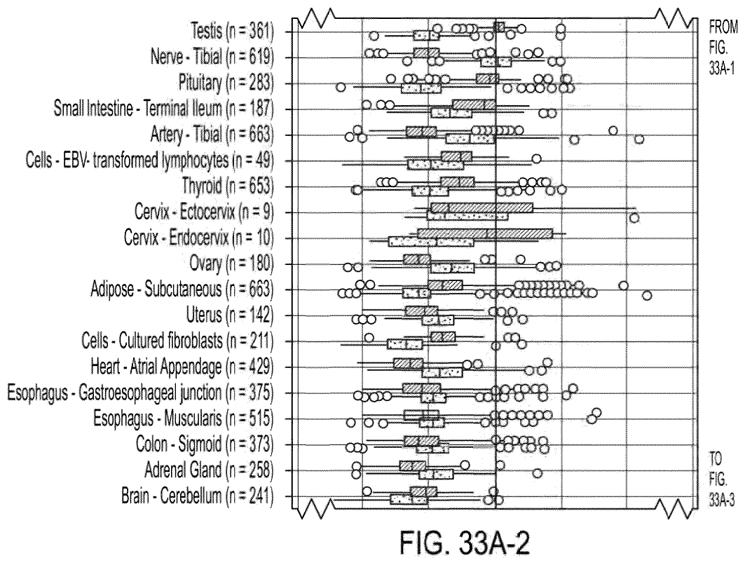

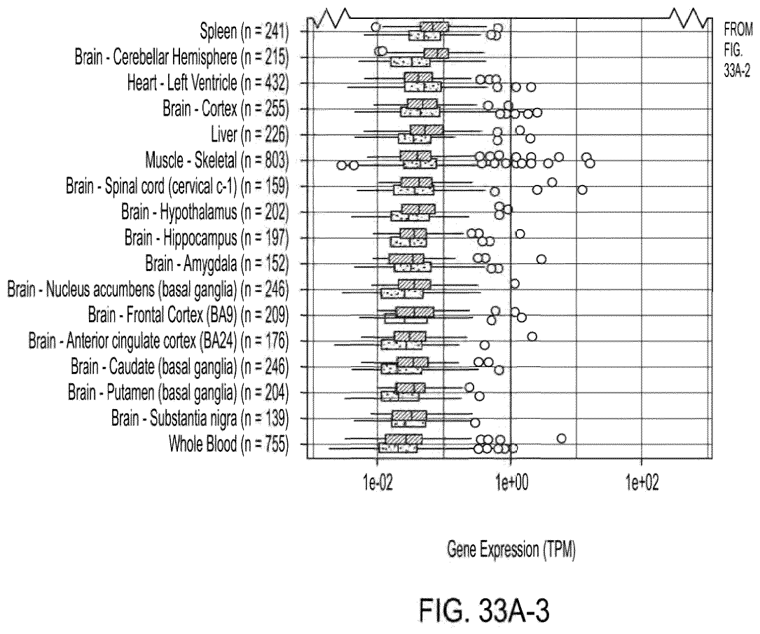

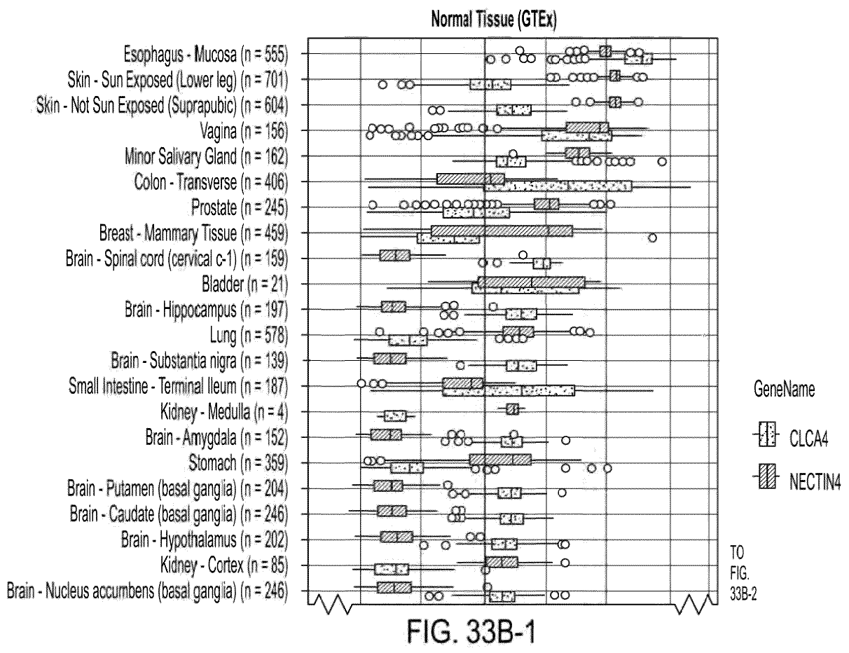

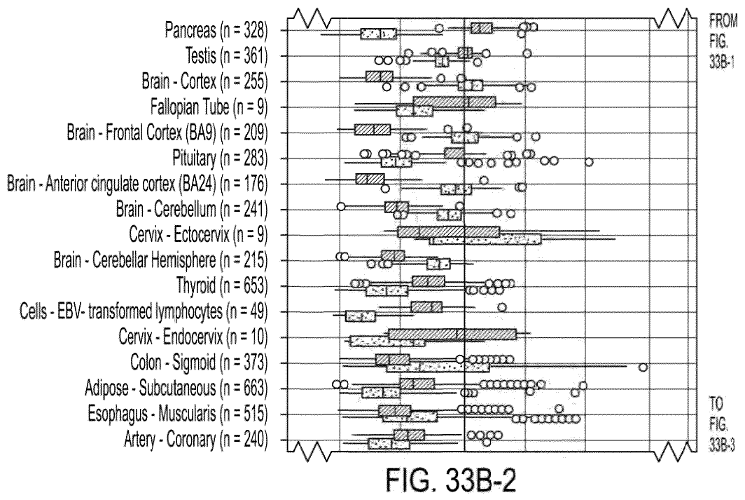

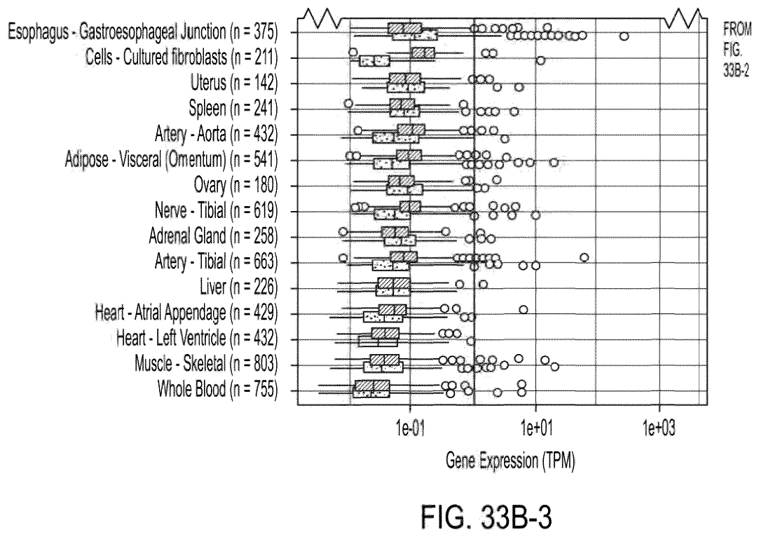

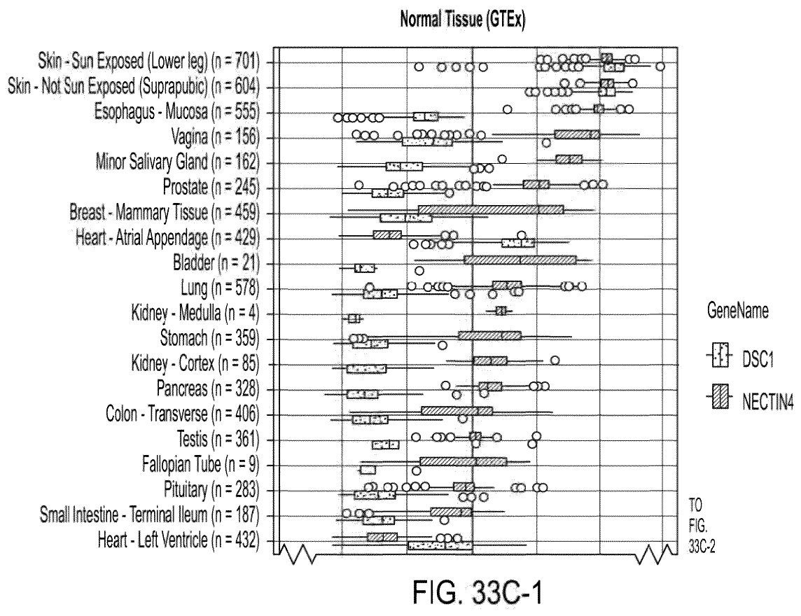

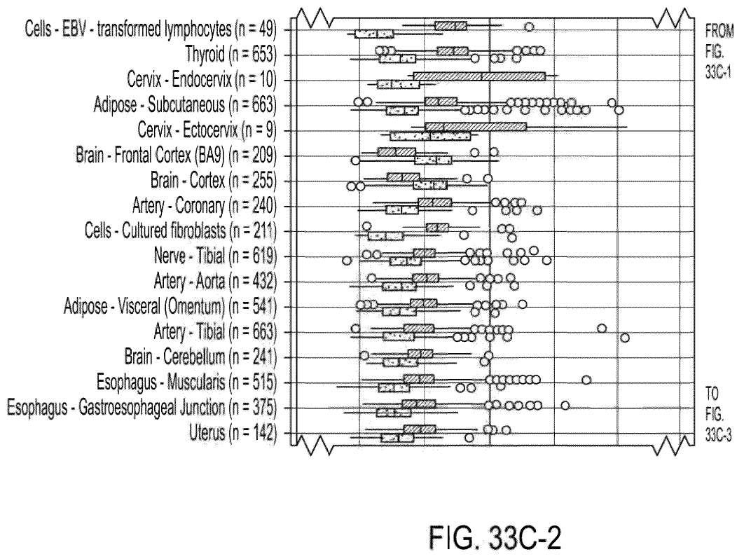

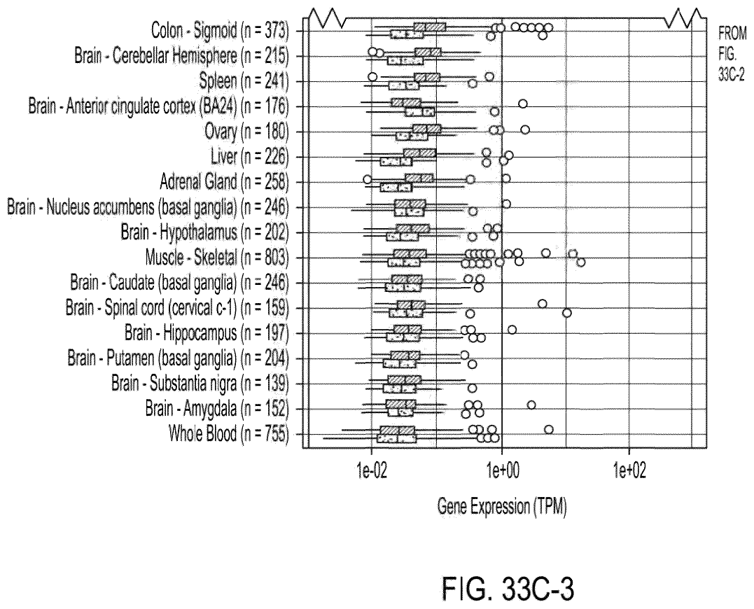

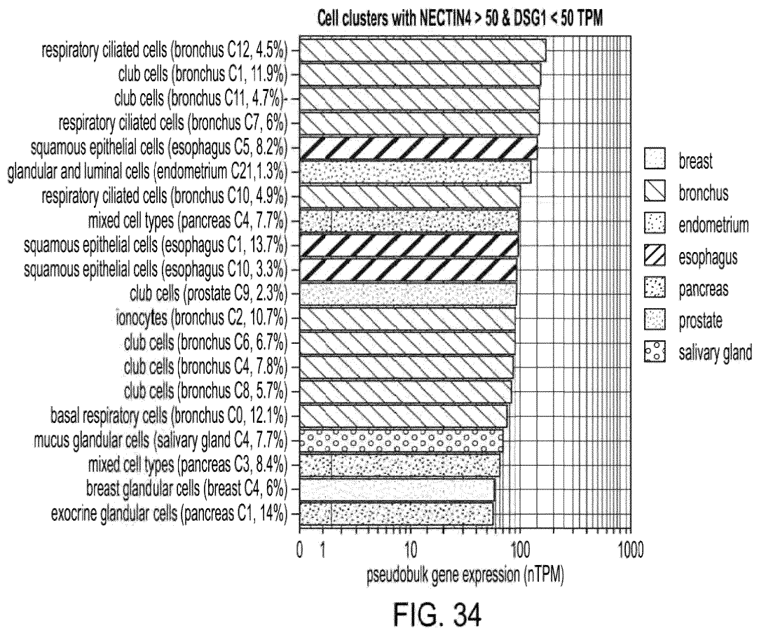

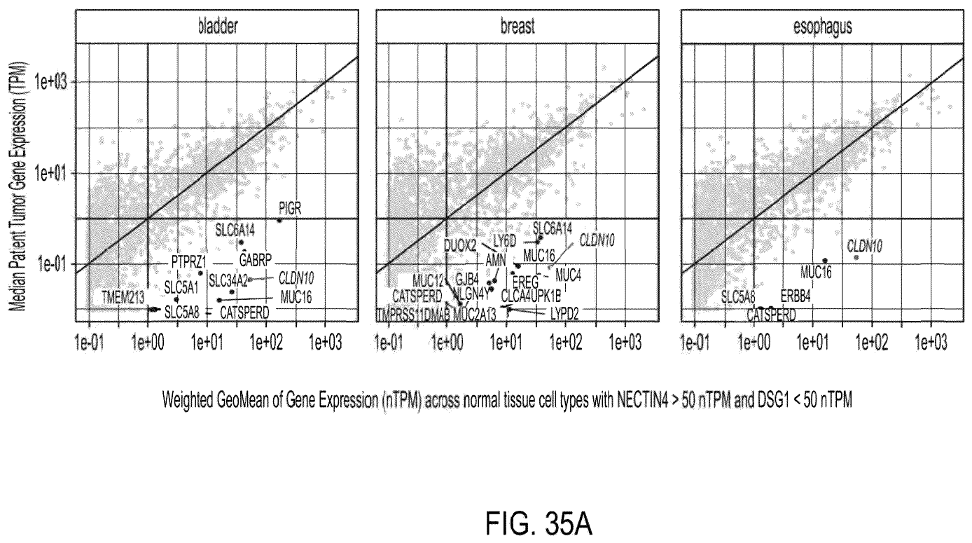

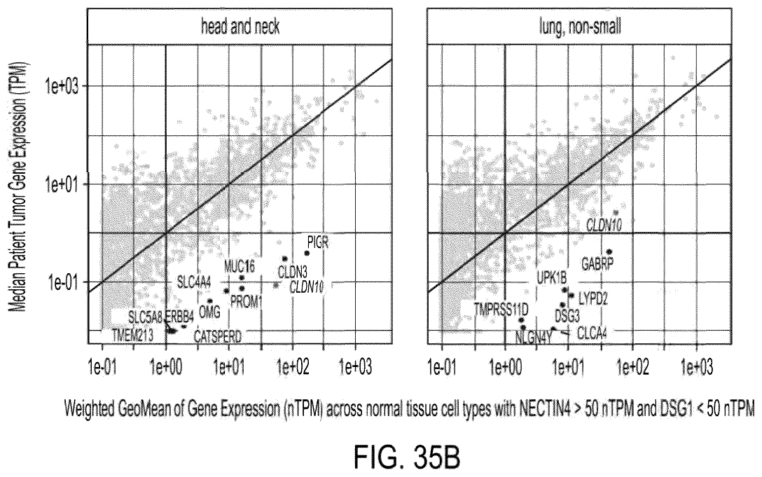

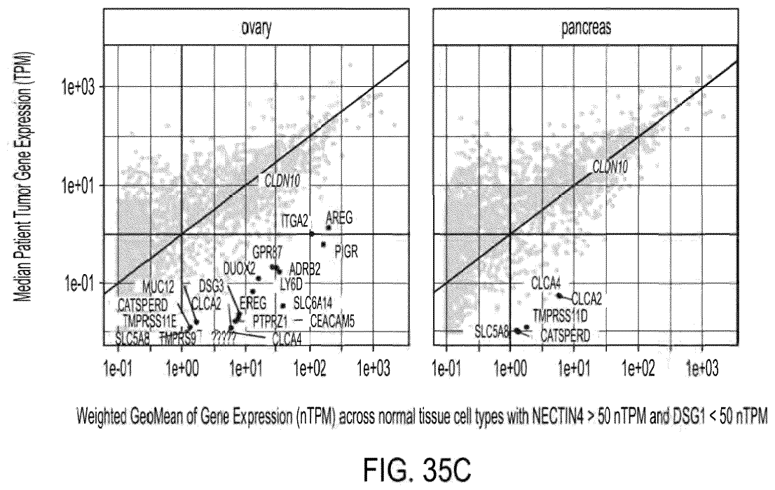

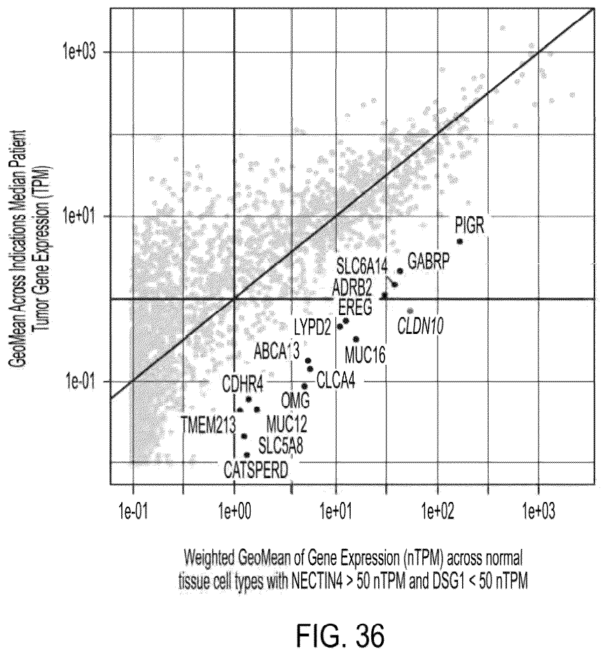

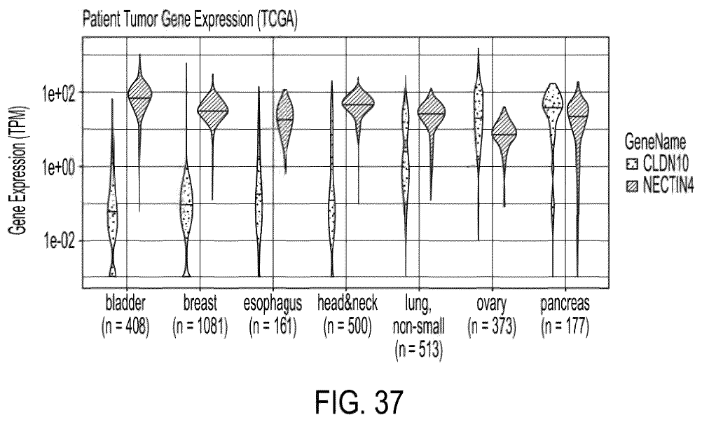

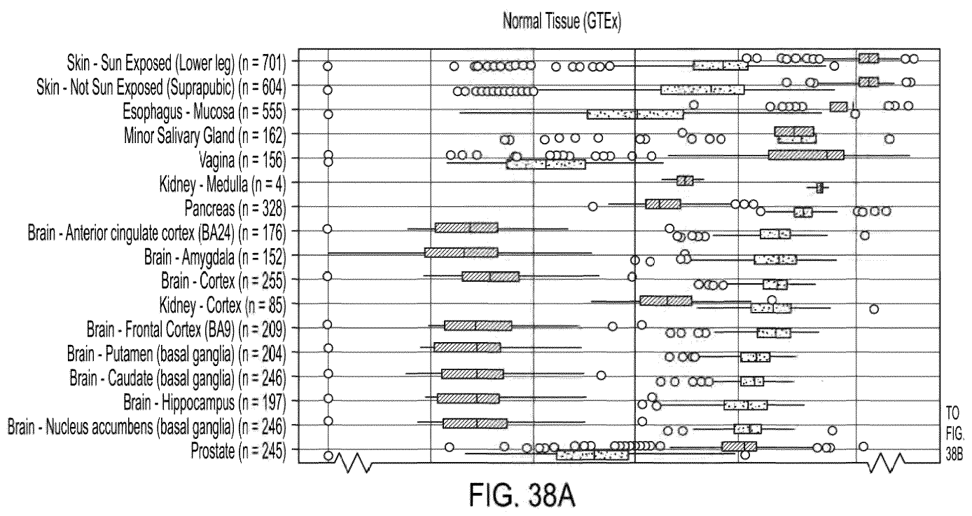

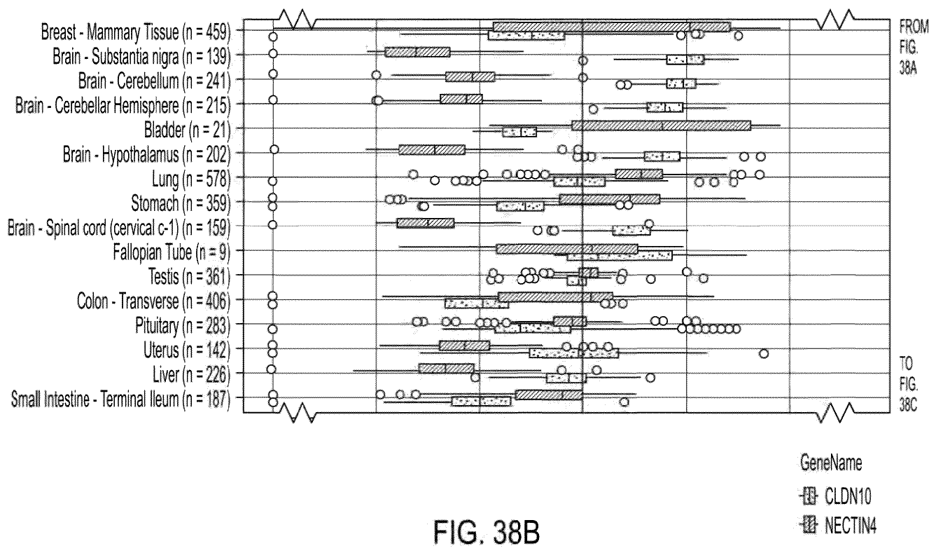

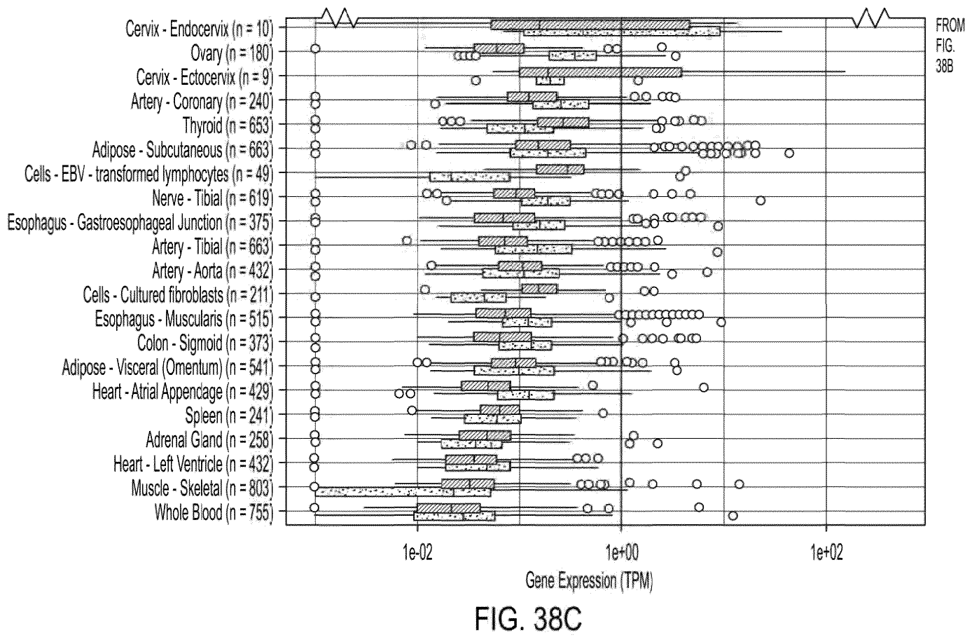

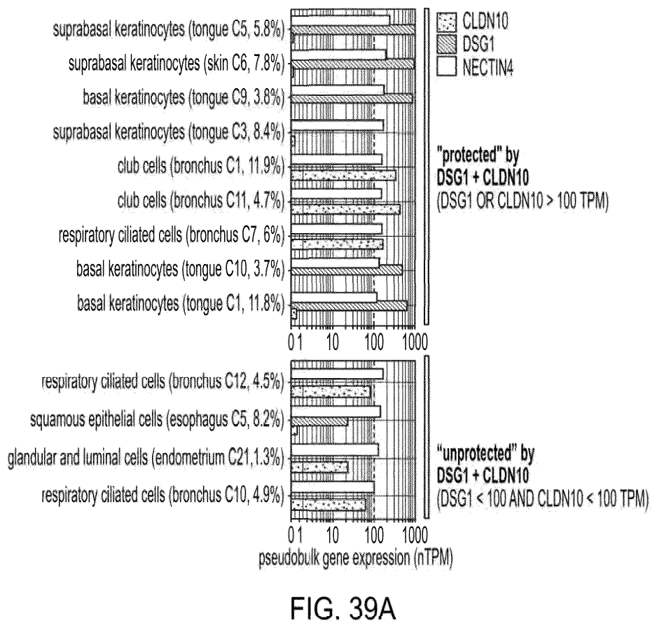

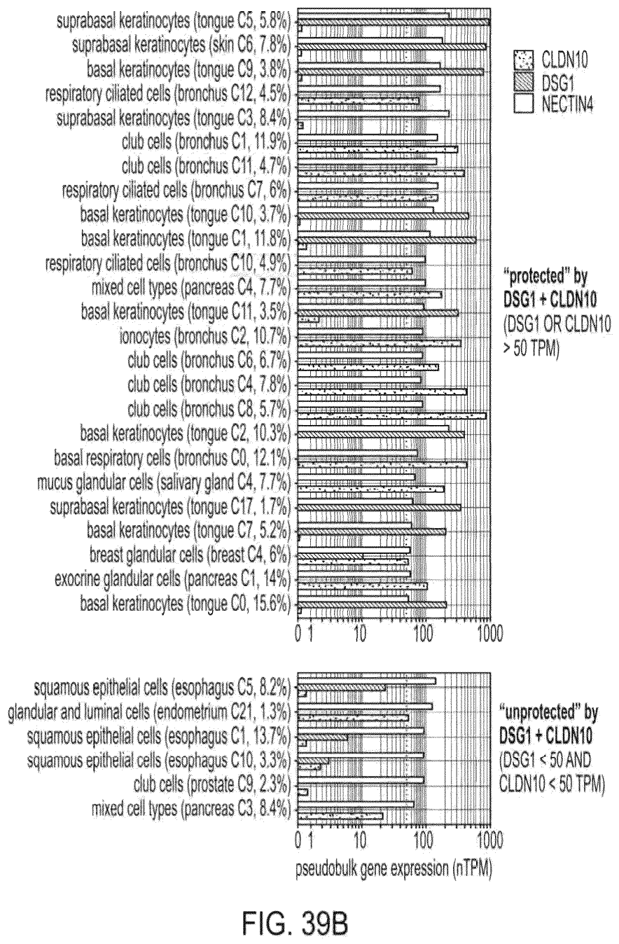

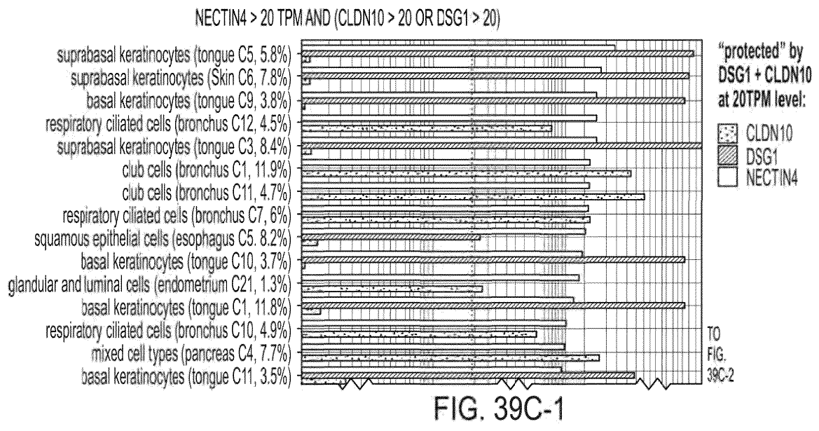

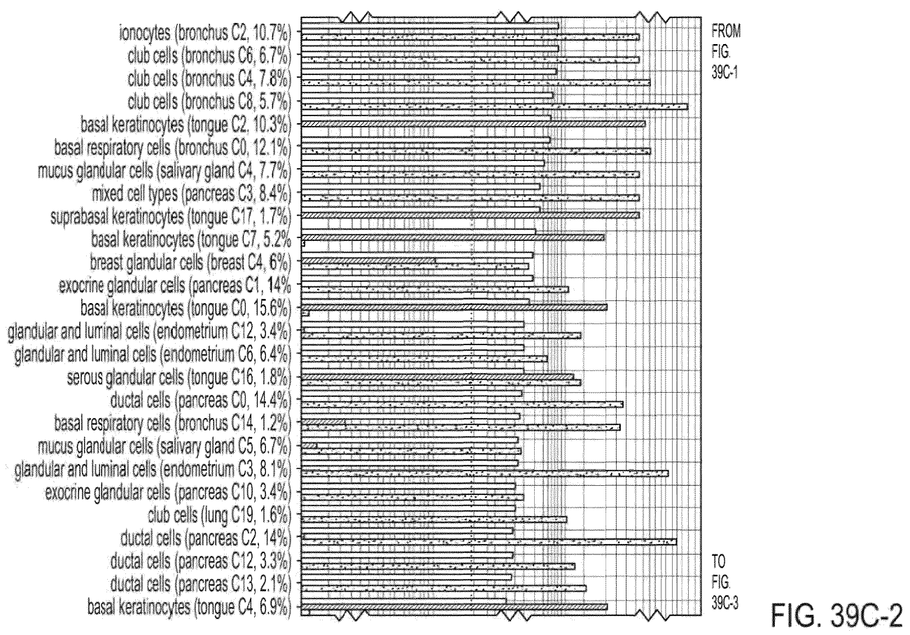

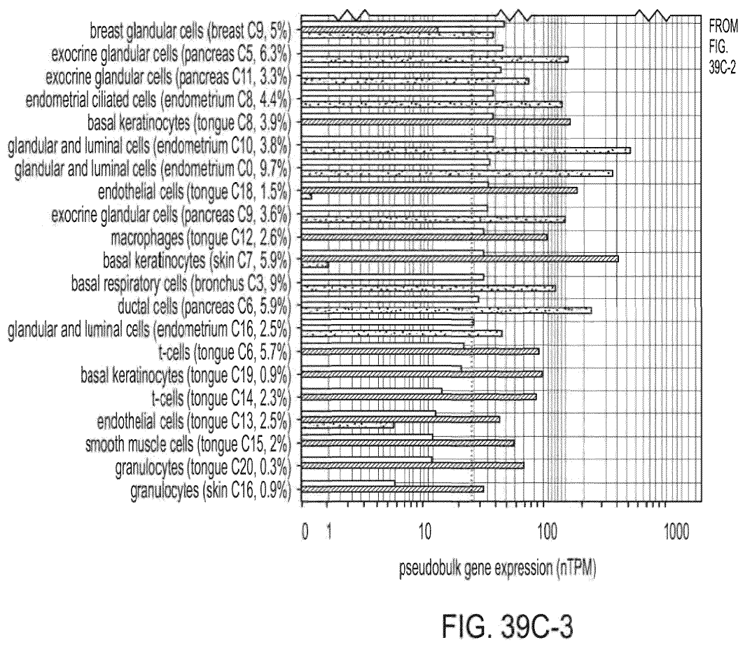

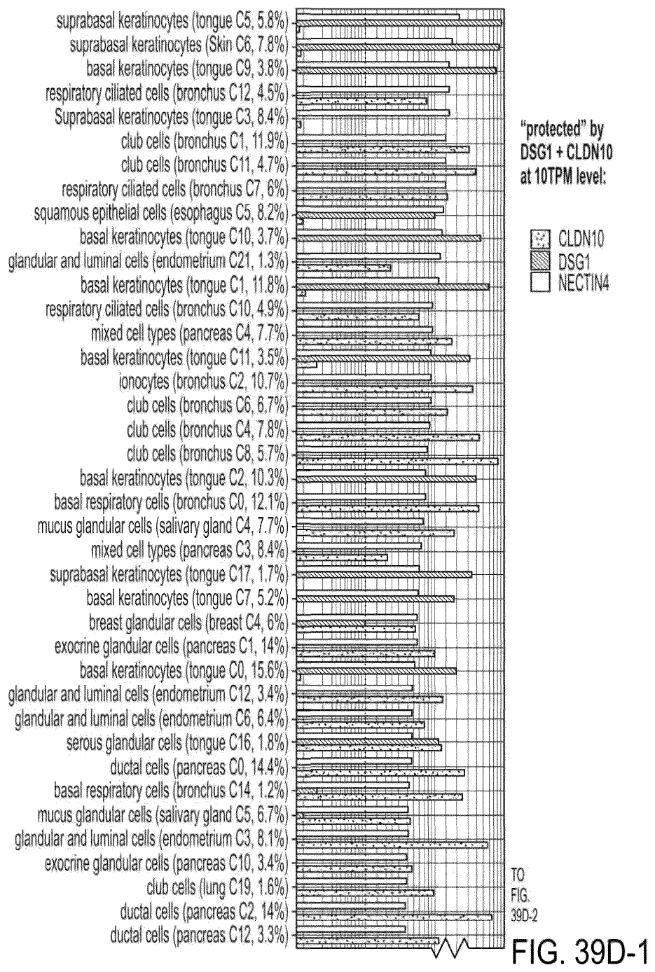

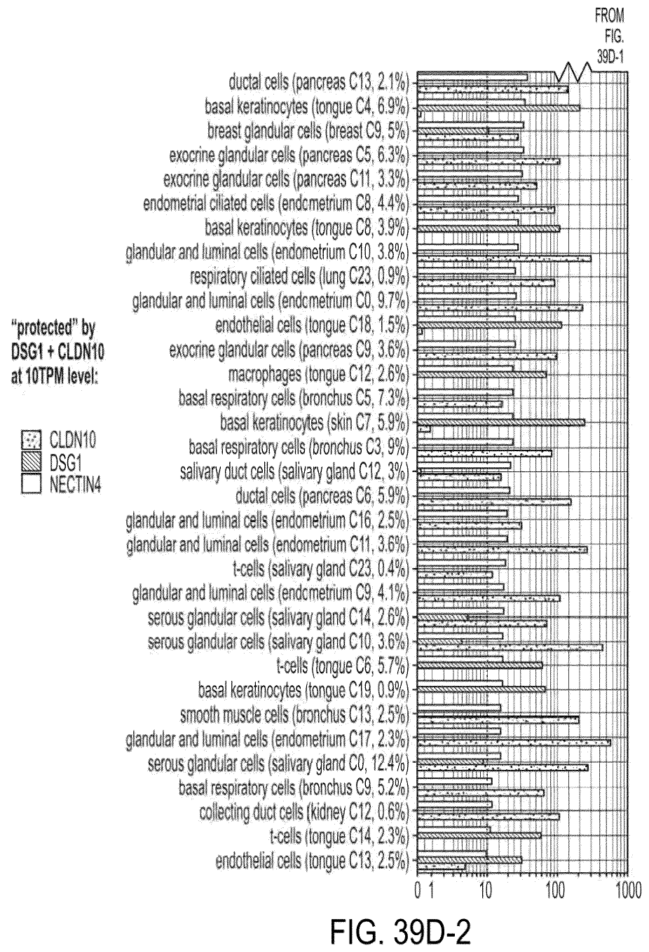

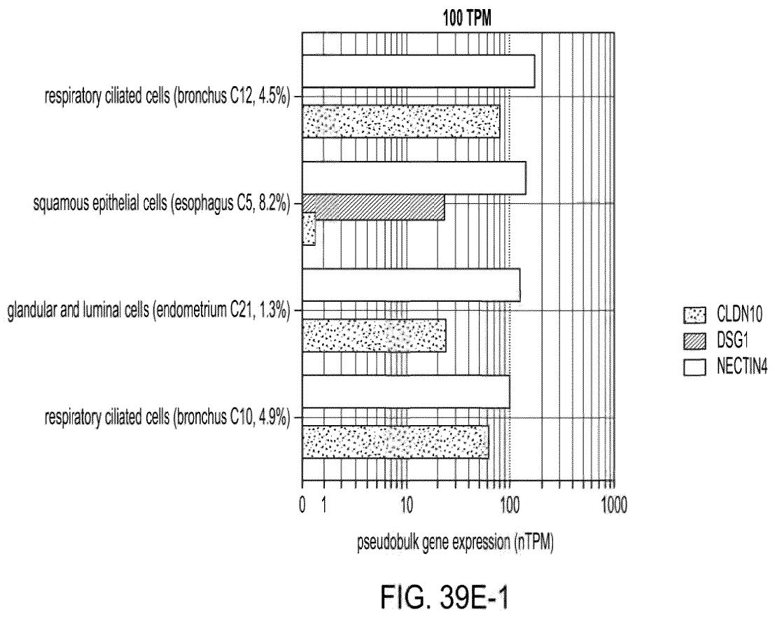

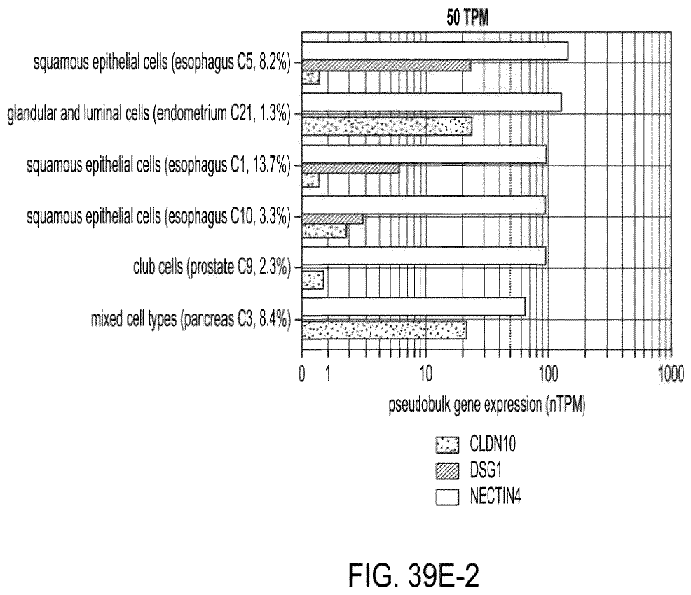

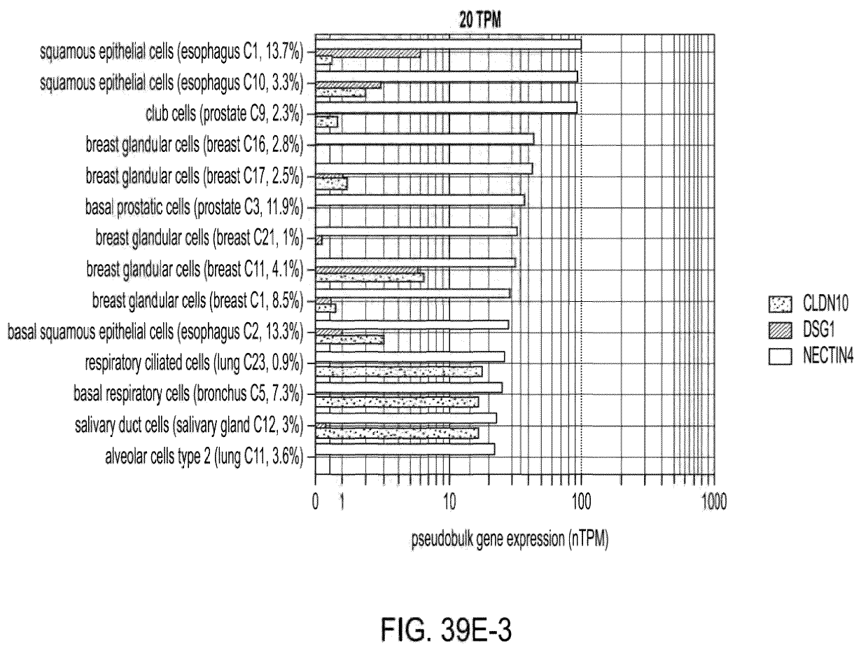

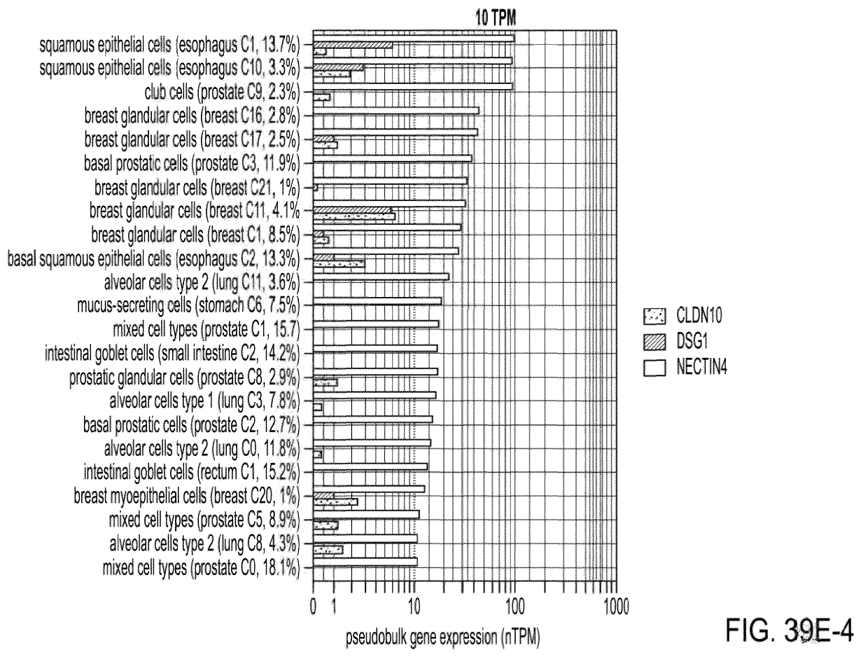

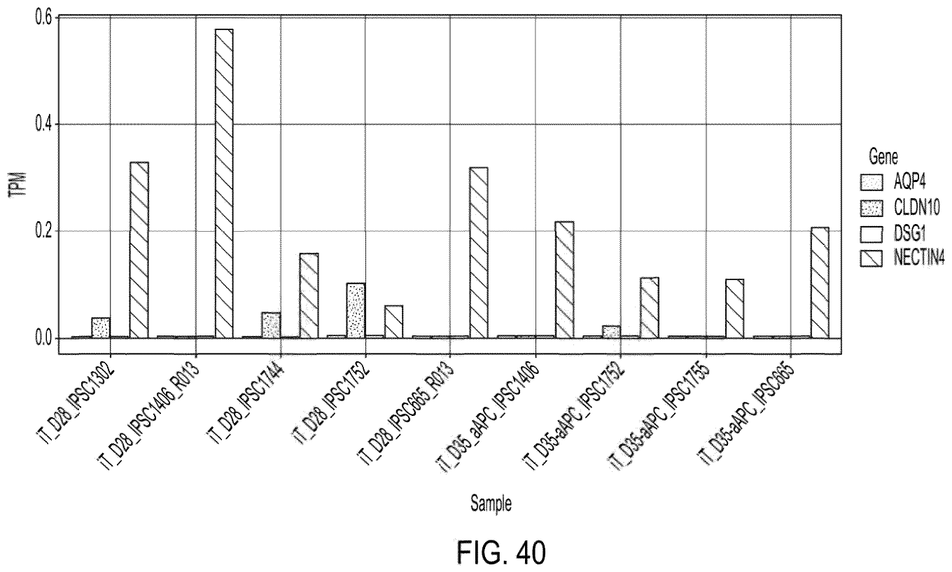

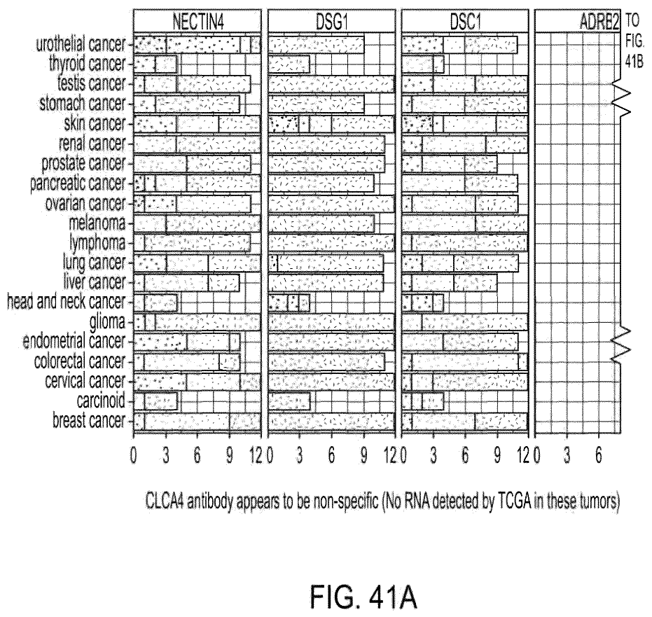

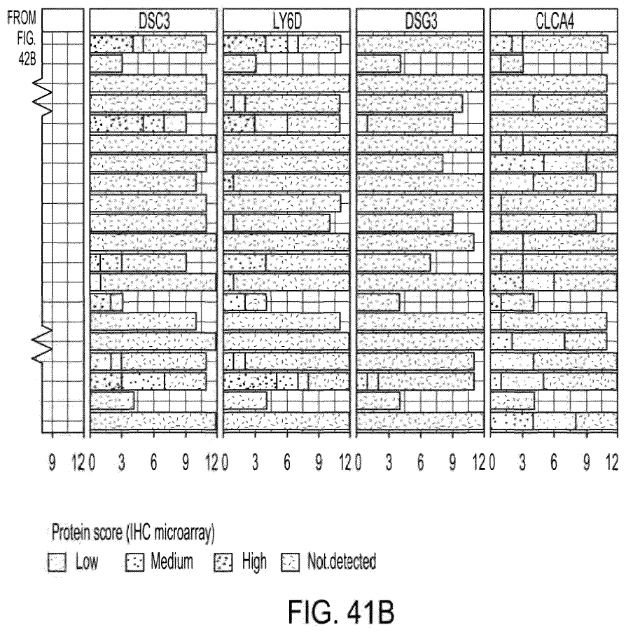

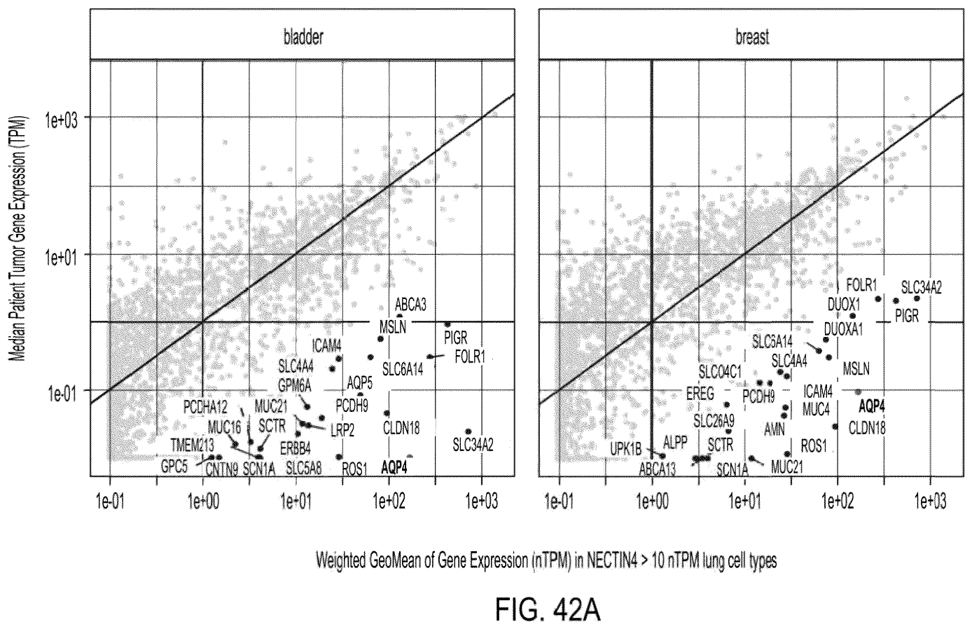

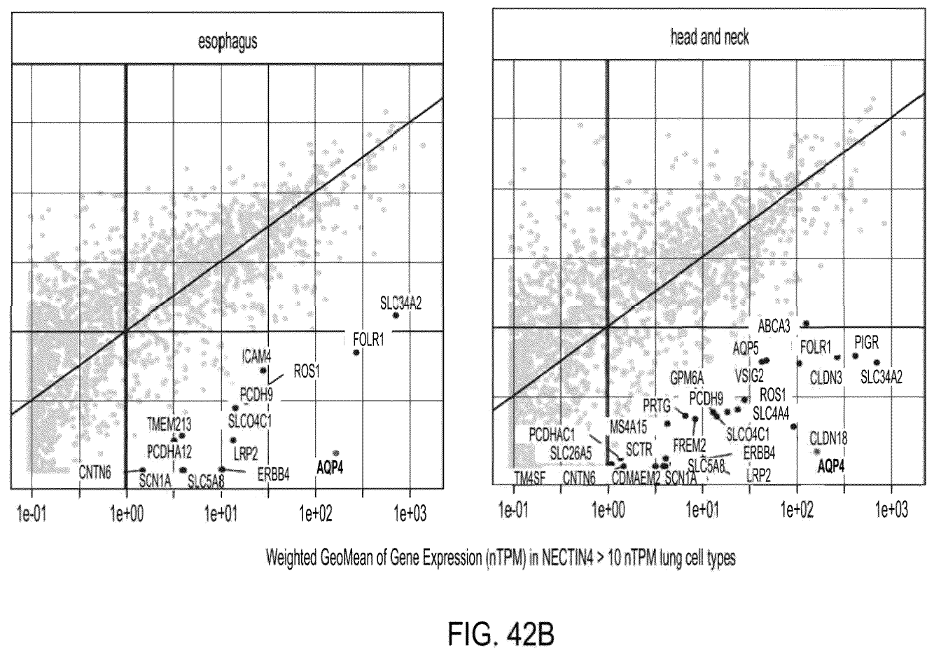

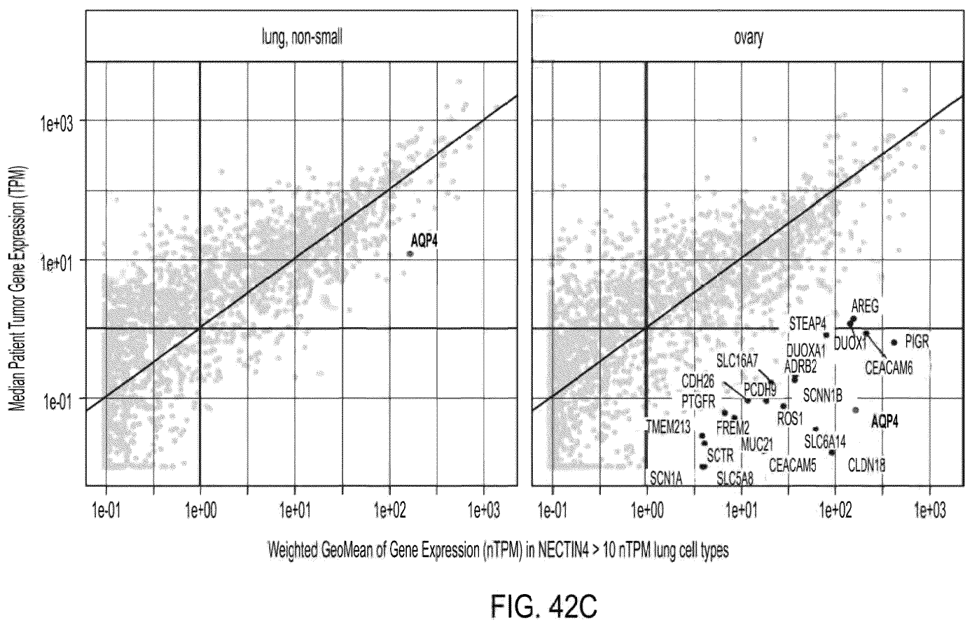

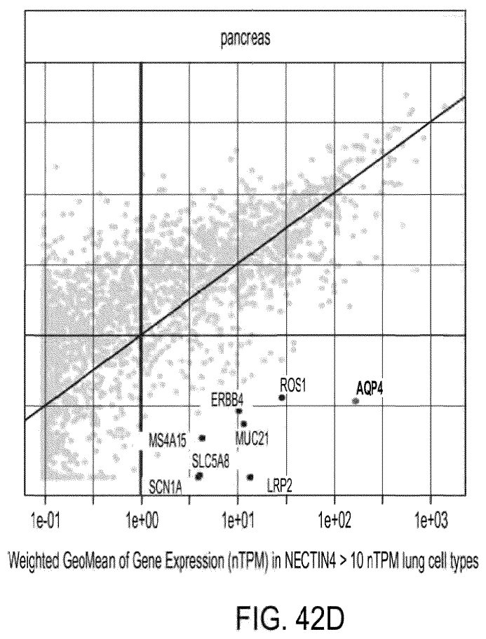

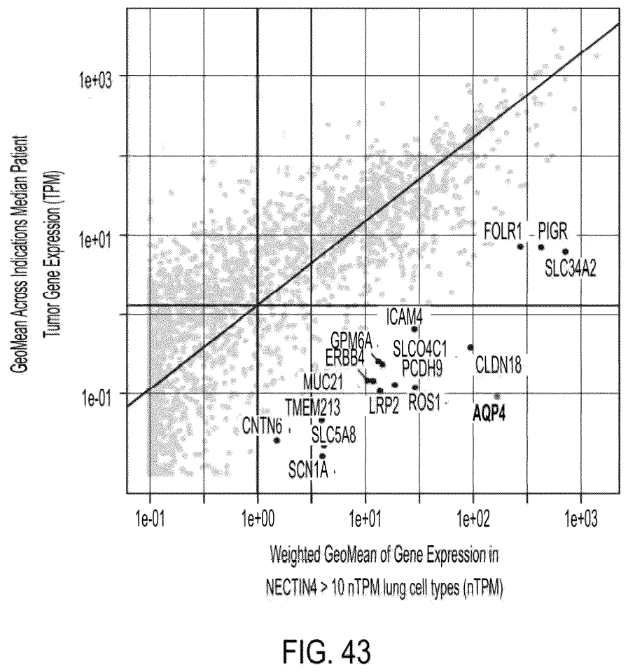

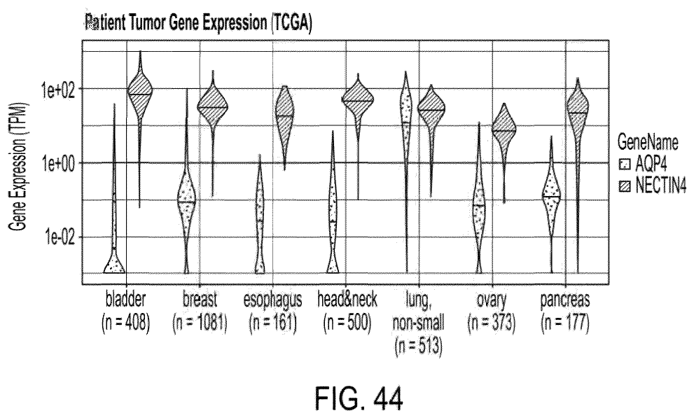

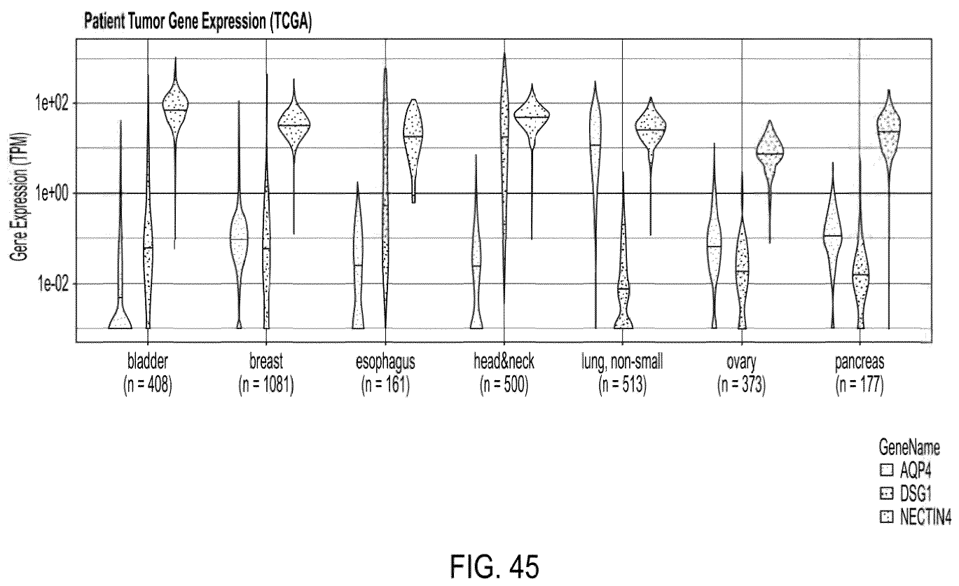

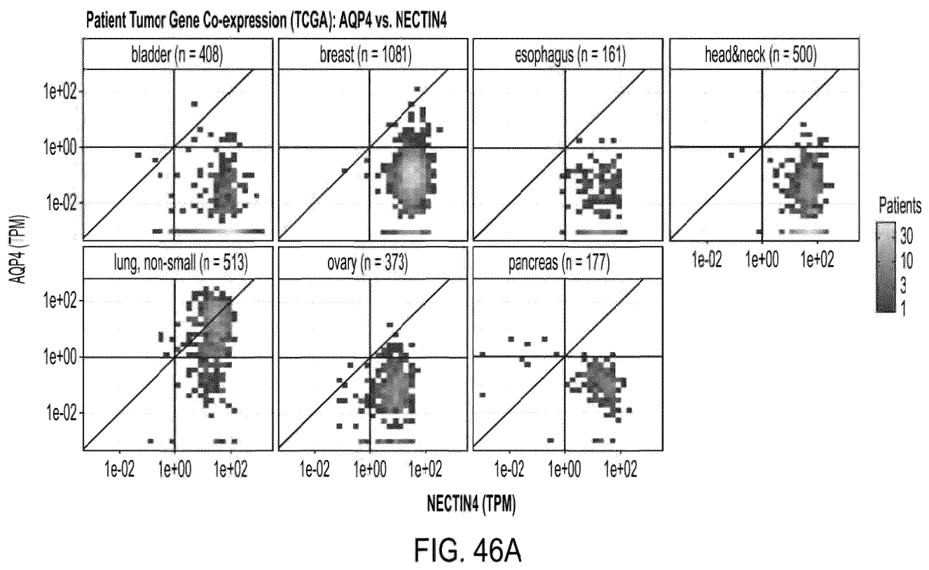

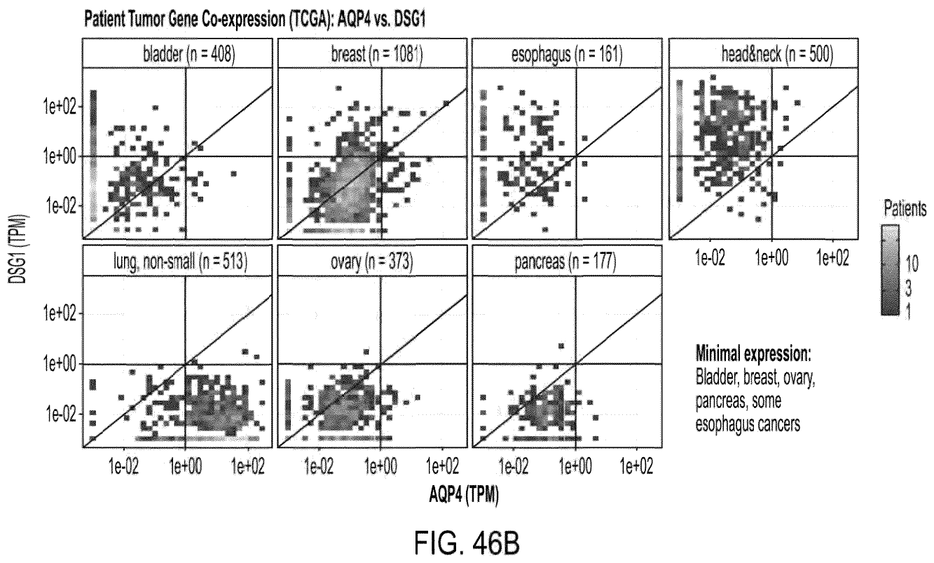

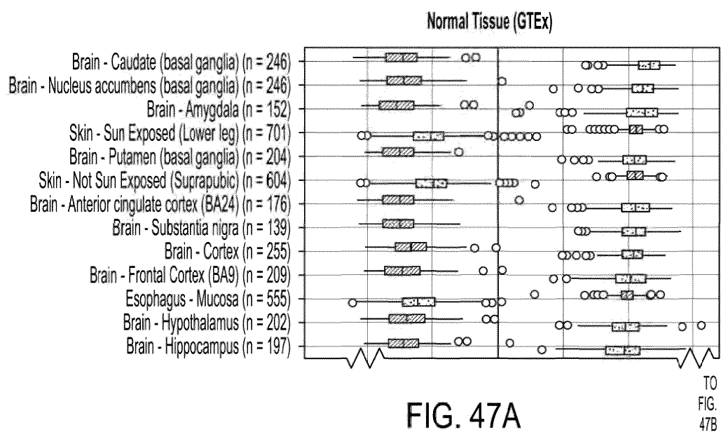

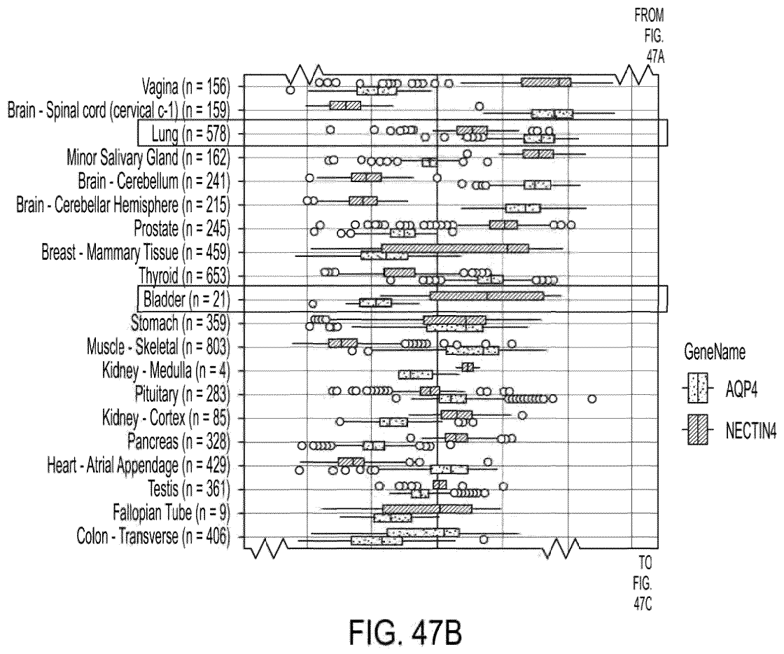

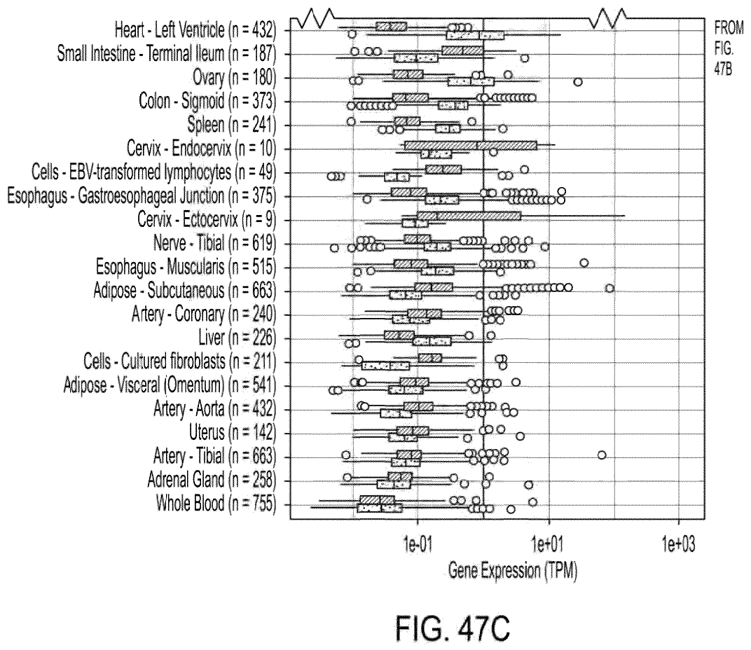

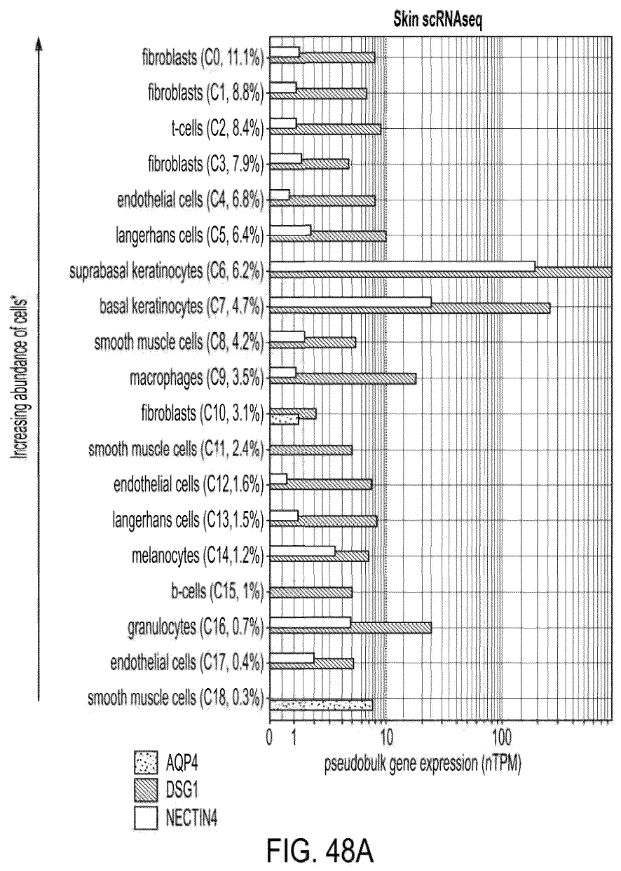

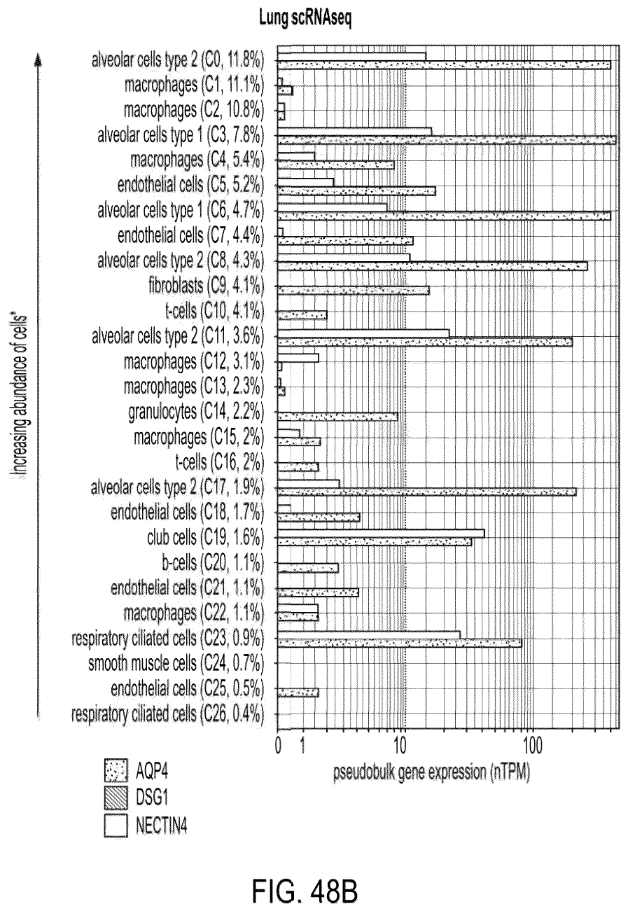

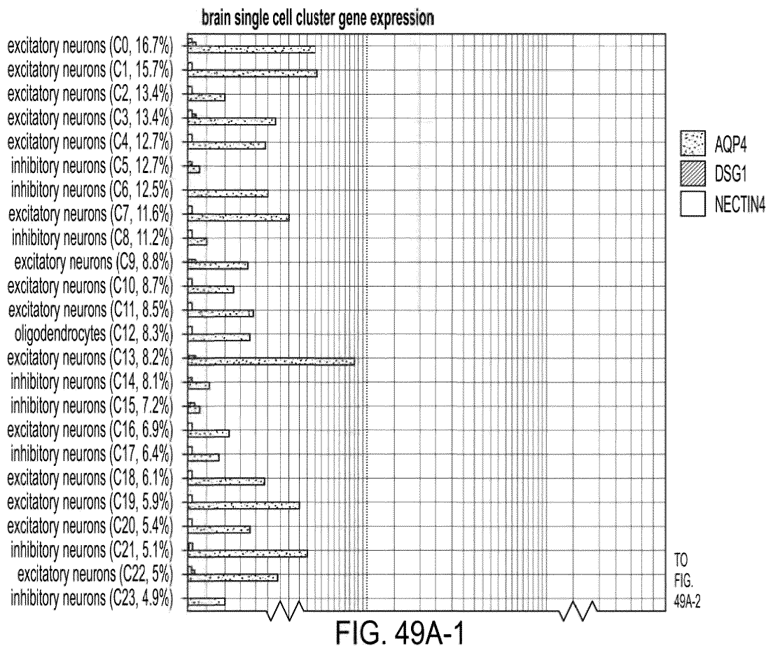

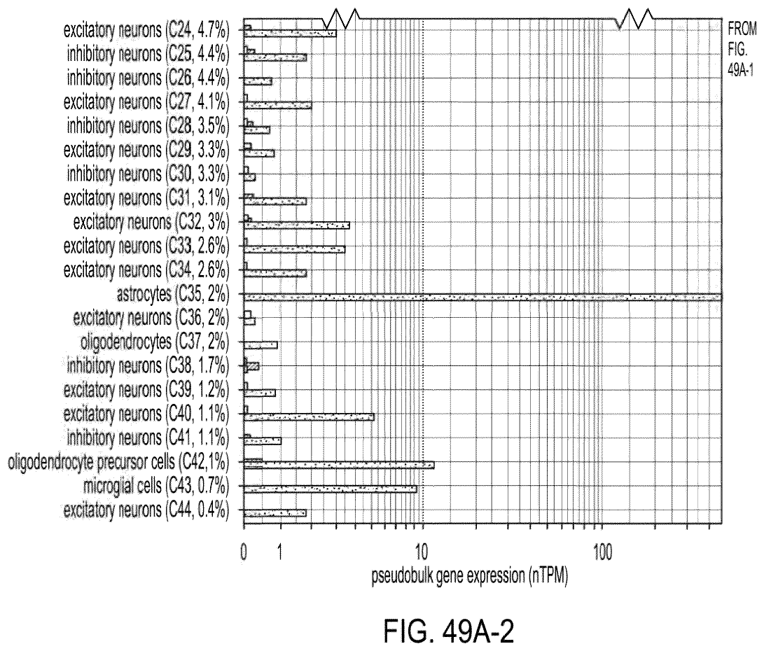

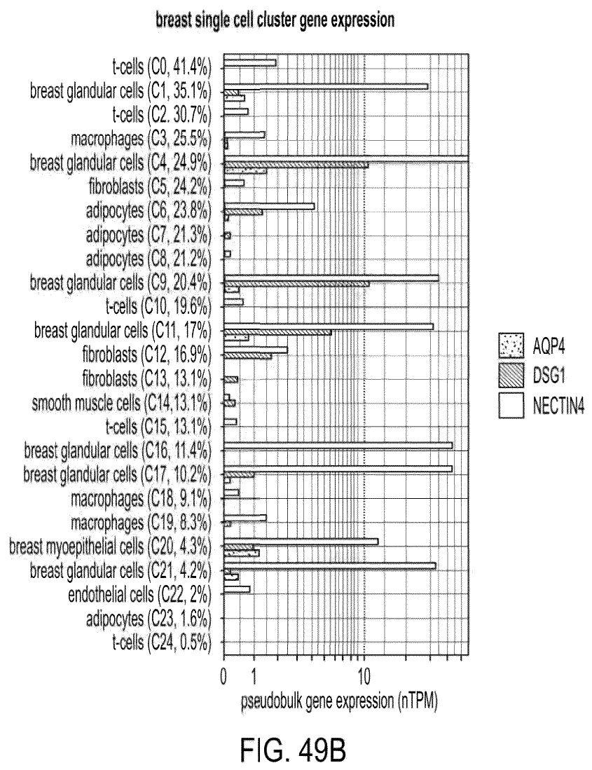

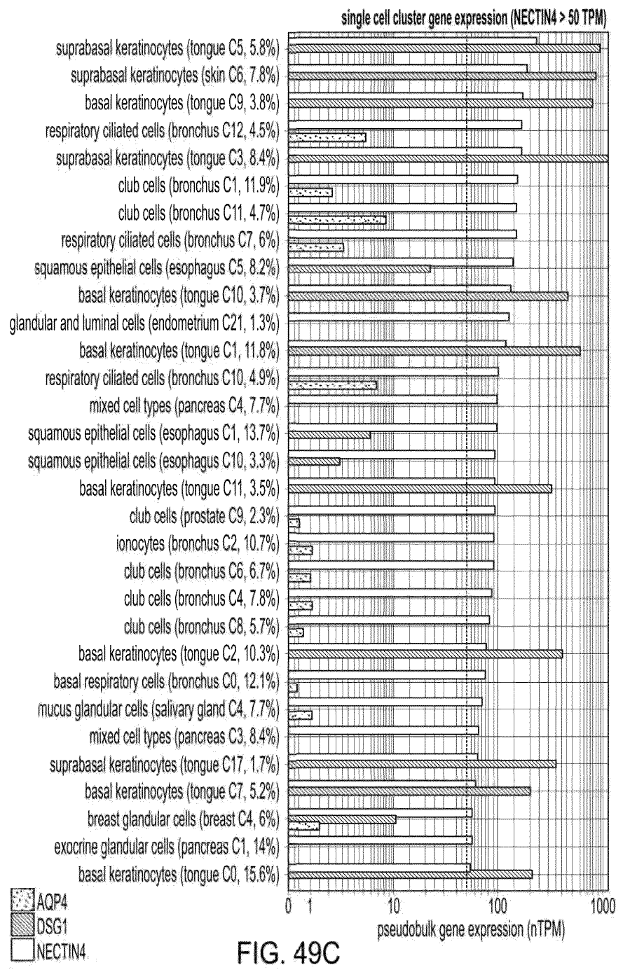

Visualization. The data was visualized as shown in FIGS. 22, 23, and 25. A violin plot was generated of DSG1 and Nectin4 patient tumor gene expression for comparison of expression distributions (FIG. 22). A scatter plot was generated of DSG1 vs. Nectin4 gene expression in the bulk RNAseq atlas of cancer tissue data (FIG. 23). A box plot was generated of DSG1 vs. Nectin4 in all tissues in the bulk RNAseq atlas of normal tissue (FIG. 25).

### Conclusion:

DSG1 was rarely expressed at levels greater than 1 TPM in patient tumors from bladder, breast, lung, ovary, and pancreas indication (FIG. 22). Visualization of co-expression of DSG1 and NECTIN4 revealed that some patient tumors from esophagus and head & neck indications have less than 1 TPM DSG1 expression while expressing high NECTIN4 (FIG. 23). DSG1 was uniformly and highly expressed across individuals in both sun exposed skin (n=701 individuals) and skin not exposed to the sun (n=604 individuals) as evidence by the tight distribution of the DSG1 expression data (FIG. 25).

Current Nectin4 targeting antibody drug conjugate therapy, while generally well tolerated, causes severe skin adverse events in some patients, driven by on-target off-tumor toxicity against Nectin4 displaying skin keratinocytes. In accordance with the invention, a method for reducing severity of adverse events is incorporation of an inhibitory CAR, also known as a NOT-gate, but heretofore the surface protein to target with such an inhibitory CAR for a Nectin4 targeting therapy is unknown. Here, we identify DSG1 (Desmoglein-1) as a top inhibitory CAR target for Nectin4 targeting CAR-T cell therapy by conducting a multi-omics analysis of public single cell RNAseq, bulk RNAseq, and protein microarray immunohistochemistry datasets. We find that DSG1 is constitutively and uniformly displayed on the surface of skin keratinocytes in the layers of epidermis where they display Nectin4, and DSG1 gene expression in normal skin is uniformly high across individuals. Conversely, DSG1 mRNA and protein are rarely expressed in Nectin4 expressing cancer indications including bladder, breast, non-small cell lung, ovarian, and pancreatic. Further, autoimmune antibodies specific to DSG1 have been described, suggesting that DSG1 is targetable by a CAR in situ. Finally, DSG1 is not expressed by the iPSC-derived CAR-T cells of the invention. While Nectin4 expression is greatest in skin keratinocytes, epithelial cells in other tissues express Nectin4 at reduced levels. We analyze Nectin4 expression in tissues and cell-types across the body in comparison to expression levels of targets associated with tissue and cell-type specific on-target off-tumor toxicity in primary CAR-T cell clinical trials.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An induced pluripotent stem cell (iPSC) or a derivative cell thereof comprising:
one or more exogenous polynucleotides encoding a chimeric antigen receptor (CAR) comprising an antigen binding domain targeting a Nectin4 antigen; and
at least one of:
(i) a deletion or reduced expression of one or more of B2M, TAP 1, TAP 2, Tapasin, RFXANK, CIITA, RFX5, RFXAP genes;
(ii) an exogenous polynucleotide encoding a human leukocyte antigen E (HLA-E) and/or human leukocyte antigen G (HLA-G);
(iii) an exogenous polynucleotide encoding a natural killer (NK) cell receptor immunoglobulin gamma Fc region receptor III (FcγRIII), cluster of differentiation 16 (CD16) and/or an NKG2D protein;
(iv) a deletion or reduced expression of one or more of NKG2A or CD70, CD38, and CD33 genes;
(v) an exogeneous polynucleotide encoding a cytokine;
(vi) an exogenous polynucleotide encoding a safety switch;
(vii) an exogeneous polynucleotide encoding a PSMA cell tracer; and
(viii) an exogeneous polynucleotide encoding a membrane bound IL-12 polypeptide;
optionally wherein:
(a) the CAR is a dual-targeting CAR comprising an additional antigen-binding domain that specifically binds to an antigen selected from the group consisting of CD70, Folate Receptor alpha, FSHR, mesothelin, and SLITRK6; or
(b) the cell comprises one or more exogenous polynucleotides encoding an additional CAR comprising an antigen-binding domain that specifically binds to an antigen selected from the group consisting of CD70, Folate Receptor alpha, FSHR, mesothelin, and SLITRK6.

2. (canceled)

3. The iPSC or the derivative cell thereof according to claim 1, wherein the CAR comprises an anti-Nectin4 VHH domain.

4.-20. (canceled)

21. The iPSC or the derivative cell thereof according to claim 1, wherein one or more of the exogenous polynucleotides are integrated at one or more loci on the chromosome of the cell selected from the group consisting of AAVS1, CLYBL, CCR5, ROSA26, collagen, HTRP, Hl 1, GAPDH, RUNX1, B2M, TAPI, TAP2, Tapasin, NLRC5, RFXANK, CIITA, RFX5, RFXAP, TCR a or b constant region, NKG2A, NKG2D, CD33, CD38, CD70, TRAC, CIS, CBL-B, SOCS2, PD1, CTLA4, LAG3, TIM3, and TIGIT genes, provided at least one of the exogenous polynucleotides is integrated at a locus of a gene selected from the group consisting of B2M, TAP 1, TAP 2, Tapasin, RFXANK, CIITA, RFX5 and RFXAP genes to thereby result in a deletion or reduced expression of the gene.

22.-24. (canceled)

25. The iPSC of claim 1, wherein the iPSC is:
(i) reprogrammed from whole peripheral blood mononuclear cells (PBMCs); or
(ii) derived from a re-programmed T-cell.

26. (canceled)

27. The iPSC or the derivative cell thereof according to claim 1, wherein the CAR comprises:
(i) a signal peptide;
(ii) an extracellular domain comprising a binding domain that specifically binds the Nectin4 antigen;
(iii) a hinge region;
(iv) a transmembrane domain;
(v) an intracellular signaling domain; and
(vi) a co-stimulatory domain,
wherein the extracellular domain comprises a VHH single domain antibody that specifically binds the Nectin4 antigen comprising amino acids having at least 90%, 91%, 92%, 93%, 94%, 95%, 96%, 97%, 98%, 99% or 100% sequence identity to one of SEQ ID NOs: 105-130 or is encoded by a polynucleotide having at least 90%, 91%, 92%, 93%, 94%, 95%, 96%, 97%, 98%, 99% or 100% sequence identity to one of SEQ ID NOs: 131-156.

28.-30. (canceled)

31. The iPSC or the derivative cell thereof according to claim 1, wherein the additional CAR comprises:
(i) a signal peptide;
(ii) an additional extracellular domain comprising a binding domain that specifically binds to an antigen selected from the group consisting of CD70, Folate Receptor alpha, FSHR, mesothelin, and SLITRK6;
(iii) a hinge region;
(iv) a transmembrane domain;
(v) an intracellular signaling domain; and
(vi) a co-stimulatory domain.

32. The iPSC or the derivative cell thereof according to claim 31, wherein:
(i) the additional extracellular domain comprises a VHH or an scFv that specifically binds to an antigen selected from the group consisting of CD70, Folate Receptor alpha, FSHR, mesothelin, and SLITRK6;
(ii) the signal peptide comprises a GMCSFR signal peptide or a MARS signal peptide;
(iii) the hinge region for each of the CAR and the additional CAR are independently selected from the group consisting of a CD28 hinge region, an IgG4 hinge region, and a CD8 hinge region;
(iv) the transmembrane domain for each of the CAR and the additional CAR are independently selected from the group consisting of a CD28 transmembrane domain and a CD8 transmembrane domain;
(v) the intracellular signaling domain comprises a CD3 intracellular domain; and/or
(vi) the co-stimulatory domain for each of the CAR and the additional CAR are independently selected from the group consisting of a CD28 signaling domain, a 41BB signaling domain, and a DAP10 signaling domain:
wherein the CAR is encoded by a polynucleotide having at least 90%, 91%, 92%, 93%, 94%, 95%, 96%, 97%, 98%, 99% or 100% sequence identity to one of SEQ ID NOs: 171-184.

33.-37. (canceled)

38. The iPSC or the derivative cell thereof according to claim 1, wherein in the CAR:
(i) the signal peptide comprises amino acids having at least 90%, 91%, 92%, 93%, 94%, 95%, 96%, 97%, 98%, 99% or 100% sequence identity to SEQ ID NOs: 1, 97, or 98;
(ii) the extracellular domain comprises amino acids having at least 90%, 91%, 92%, 93%, 94%, 95%, 96%, 97%, 98%, 99% or 100% sequence identity to SEQ ID NOs: 105-130, or the extracellular domain is encoded by a polynucleotide having at least 90%, 91%, 92%, 93%, 94%, 95%, 96%, 97%, 98%, 99% or 100% sequence identity to SEQ ID NOs: 131-156;
(iii) the hinge region comprises amino acids having at least 90%, 91%, 92%, 93%, 94%, 95%, 96%, 97%, 98%, 99% or 100% sequence identity to SEQ ID NO: 21 or 96;
(iv) the transmembrane domain comprises amino acids having at least 90%, 91%, 92%, 93%, 94%, 95%, 96%, 97%, 98%, 99% or 100% sequence identity to SEQ ID NO: 23 or 24;
(v) the intracellular signaling domain comprises amino acids having at least 90%, 91%, 92%, 93%, 94%, 95%, 96%, 97%, 98%, 99% or 100% sequence identity to SEQ ID NO: 6, or the intracellular signaling domain is encoded by a polynucleotide having at least 90%, 91%, 92%, 93%, 94%, 95%, 96%, 97%, 98%, 99% or 100% sequence identity to SEQ ID NO: 101; and
(vi) the co-stimulatory domain comprises amino acids having at least 90%, 91%, 92%, 93%, 94%, 95%, 96%, 97%, 98%, 99% or 100% sequence identity to SEQ ID NO: 8 or 17.

39.-55. (canceled)

56. The iPSC or the derivative cell thereof according to claim 1 wherein
the one or more exogenous polynucleotides encoding a chimeric antigen receptor (CAR) comprising an antigen binding domain targeting a Nectin4 antigen comprises nucleotides having at least 90%, 91%, 92%, 93%, 94%, 95%, 96%, 97%, 98%, 99% or 100% sequence identity to one or more selected from the group consisting of SEQ ID NOs: 171-184.

57. (canceled)

58. The iPSC or the derivative cell thereof according to claim 56, wherein the exogenous polynucleotides are integrated into a gene locus independently selected from the group consisting of an AAVS1 locus, a B2M locus, a CIITA locus, a CCR5 locus, a CD70 locus, a CLYBL locus, an NKG2A locus, an NKG2D locus, a CD33 locus, a CD38 locus, a TRAC locus, a TRBC1 locus, a ROSA26 locus, an HTRP locus, a GAPDH locus, a RUNX1 locus, a TAPI locus, a TAP2 locus, a TAPBP locus, an NLRC5 locus, a RFXANK locus, a RFX5 locus, a RFXAP locus, a CISH locus, a CBLB locus, a SOCS2 locus, a PD1 locus, a CTLA4 locus, a LAG3 locus, a TIM3 locus, and a TIGIT locus.

59. The iPSC or the derivative cell thereof according to claim 58, wherein:
(i) the one or more exogenous polynucleotides encoding a chimeric antigen receptor (CAR) comprising an antigen binding domain targeting a Nectin4 antigen is integrated at a locus of the TRAC gene; and
(ii) there is a deletion or reduced expression of the B2M and CIITA gene.

60.-78. (canceled)

79. The derivative cell of claim 1, wherein the derivative cell is:
(i) a natural killer (NK) cell;
(ii) a T cell;
(iii) a gamma delta T cell; or
(iv) a gamma delta Vγ9/Vδ1 T cell.

80.-82. (canceled)

83. A composition comprising the derivative cell according to claim 1, optionally further comprising or being used in combination with, one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD).

84. (canceled)

85. A CD34+ hematopoietic progenitor cell (HPC) derived from an induced pluripotent stem cell (iPSC) of claim 1.

86.-87. (canceled)

88. The CD34+ HPC according to claim 85, wherein one or more of the exogenous polynucleotides are integrated at one or more loci on the chromosome of the cell independently selected from the group consisting of AAVS1, CLYBL, CCR5, ROSA26, collagen, HTRP, Hl 1, GAPDH, RUNX1, B2M, TAPI, TAP2, Tapasin, NLRC5, RFXANK, CIITA, RFX5, RFXAP, TCR a or b constant region, NKG2A, NKG2D, CD33, CD38, CIS, CBL-B, SOCS2, PD1, CTLA4, LAG3, TIM3, and TIGIT genes, provided at least one of the exogenous polynucleotides is integrated at a locus of a gene selected from the group consisting of B2M, TAP 1, TAP 2, Tapasin, RFXANK, CIITA, RFX5 and RFXAP genes to thereby result in a deletion or reduced expression of the gene.

89.-90. (canceled)

91. The CD34+ HPC according to claim 85, wherein the CAR comprises:
(i) a signal peptide;
(ii) an extracellular domain comprising a binding domain that specifically binds the Nectin4 antigen;
(iii) a hinge region;
(iv) a transmembrane domain;
(v) an intracellular signaling domain; and
(vi) a co-stimulatory domain,
wherein the extracellular domain comprises a VHH single domain antibody that specifically binds the Nectin4 antigen comprising amino acids having at least 90%, 91%, 92%, 93%, 94%, 95%, 96%, 97%, 98%, 99% or 100% sequence identity to one of SEQ ID NOs: 105-130.

92.-93. (canceled)

94. The CD34+ HPC according to claim 85 having an additional CAR comprising:
(i) a signal peptide;
(ii) an additional extracellular domain comprising a binding domain that specifically binds an antigen selected from the group consisting of CD70, Folate Receptor alpha, FSHR, mesothelin, and SLITRK6;
(iii) a hinge region;
(iv) a transmembrane domain;
(v) an intracellular signaling domain; and
(vi) a co-stimulatory domain, such as a co-stimulatory domain comprising a CD28 signaling domain,
wherein the additional extracellular domain comprises a VHH that specifically binds the antigen selected from the group consisting of CD70, Folate Receptor alpha, FSHR, mesothelin, and SLITRK6.

95.-100. (canceled)

101. A chimeric antigen receptor (CAR) polypeptide comprising an extracellular domain comprising an antigen binding domain that specifically binds to Nectin4, wherein the CAR comprises:
(i) a signal peptide;
(ii) the extracellular domain comprising the antigen binding domain that specifically binds to the Nectin4 antigen;
(iii) a hinge region;
(iv) one or more transmembrane domains;
(v) an intracellular signaling domain; and/or
(vi) a co-stimulatory domain
wherein the CAR is a dual-targeting CAR, and wherein the extracellular domain comprises an additional antigen-binding domain that specifically binds to an antigen selected from the group consisting of CD70, Folate Receptor alpha, FSHR, mesothelin, and SLITRK6.

102.-103. (canceled)

104. The CAR according to claim 101, wherein:
(i) the extracellular domain comprises a VHH single domain antibody that specifically binds to the Nectin4 antigen comprising amino acids having at least 90%, 91%, 92%, 93%, 94%, 95%, 96%, 97%, 98%, 99% or 100% sequence identity to one of SEQ ID NOs: 105-130, or is encoded by a polynucleotide having at least 90%, 91%, 92%, 93%, 94%, 95%, 96%, 97%, 98%, 99% or 100% sequence identity to one of SEQ ID NOs: 131-156;
(ii) the signal peptide comprises a GMCSFR signal peptide or a MARS signal peptide;
(iii) the hinge region for each of the CAR and the additional CAR are independently selected from the group consisting of a CD28 hinge region, an IgG4 hinge region, and a CD8 hinge region;
(iv) the transmembrane domain for each of the CAR and the additional CAR are independently selected from the group consisting of a CD28 transmembrane domain and a CD8 transmembrane domain;
(v) the intracellular signaling domain comprises a CD3 intracellular domain; and/or
(vi) the co-stimulatory domain for each of the CAR and the additional CAR are independently selected from the group consisting of a CD28 signaling domain, a 41BB signaling domain, and a DAP10 signaling domain.

105.-111. (canceled)

112. The CAR according to claim 101, wherein in the CAR:
(i) the signal peptide comprises amino acids having at least 90%, 91%, 92%, 93%, 94%, 95%, 96%, 97%, 98%, 99% or 100% sequence identity to SEQ ID NOs: 1, 97, or 98;
(ii) the extracellular domain comprises amino acids having at least 90%, 91%, 92%, 93%, 94%, 95%, 96%, 97%, 98%, 99% or 100% sequence identity to SEQ ID NOs: 105-130, or the extracellular domain is encoded by a polynucleotide having at least 90%, 91%, 92%, 93%, 94%, 95%, 96%, 97%, 98%, 99% or 100% sequence identity to SEQ ID NOs: 131-156;
(iii) the hinge region comprises amino acids having at least 90%, 91%, 92%, 93%, 94%, 95%, 96%, 97%, 98%, 99% or 100% sequence identity to SEQ ID NO: 21 or 96;
(iv) the transmembrane domain comprises amino acids having at least 90%, 91%, 92%, 93%, 94%, 95%, 96%, 97%, 98%, 99% or 100% sequence identity to SEQ ID NO: 23 or 24;
(v) the intracellular signaling domain comprises amino acids having at least 90%, 91%, 92%, 93%, 94%, 95%, 96%, 97%, 98%, 99% or 100% sequence identity to SEQ ID NO: 6, or the intracellular signaling domain is encoded by a polynucleotide having at least 90%, 91%, 92%, 93%, 94%, 95%, 96%, 97%, 98%, 99% or 100% sequence identity to SEQ ID NO: 101; and
(vi) the co-stimulatory domain comprises amino acids having at least 90%, 91%, 92%, 93%, 94%, 95%, 96%, 97%, 98%, 99% or 100% sequence identity to SEQ ID NO: 8 or 17;
wherein the CAR is encoded by a polynucleotide having at least 90%, 91%, 92%, 93%, 94%, 95%, 96%, 97%, 98%, 99% or 100% sequence identity to one of SEQ ID NOs: 171-184.

113.-127. (canceled)

128. A method of treating cancer in a subject in need thereof, comprising administering the derivative cell according to claim 1, or a pharmaceutical composition thereof, to a subject in need thereof, wherein:
(i) the cancer is selected from the group consisting of leukemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovarian cancer, prostate cancer, lung cancer, colorectal cancer, cervical cancer, liver cancer, head and neck cancer, esophageal cancer, pancreatic cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP); and
(ii) the subject has:
(a) minimal residual disease (MRD) after an initial cancer treatment: or
(b) no minimal residual disease (MRD) after one or more cancer treatments or repeated dosing.

129.-132. (canceled)

133. The method according to claim 128, further comprising administering to the subject a therapeutic agent selected from the group consisting of ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, avelumab, ofatumumab, panitumumab, and ustekinumab, wherein the cell and the therapeutic agent are administered concurrently or sequentially.

134.-136. (canceled)

137. A method of manufacturing the derivative cell of claim 79, comprising differentiating the iPSC under conditions for cell differentiation to thereby obtain the derivative cell, wherein the iPSC is obtained by genetically engineering an unmodified iPSC, wherein the genetic engineering comprises targeted editing of the genome of the iPSC, wherein the targeted editing comprises deletion, insertion, or in/del carried out by CRISPR, ZFN, TALEN, homing nuclease, homology recombination, or any other functional variation of these methods.

138.-139. (canceled)
